(19) United States
(12) Patent Application Publication
(10) Pub. No.: US 2026/0136750 A1
(43) Pub. Date: May 14, 2026

(54) HETEROCYCLIC COMPOUND, ORGANIC LIGHT EMITTING DEVICE COMPRISING SAME AND COMPOSITION FOR FORMING ORGANIC MATERIAL LAYER OF ORGANIC LIGHT EMITTING DEVICE

(71) Applicant: LT Materials Co., Ltd., Yongin-si (KR)

(72) Inventors: Ji-Young KIM, Yongin-si (KR); Geon-Yu PARK, Yongin-si (KR); Young-Seok NO, Yongin-si (KR); Dong-Jun KIM, Yongin-si (KR); Dae-Hyuk CHOI, Yongin-si (KR)

(21) Appl. No.: 19/439,428

(22) Filed: Jan. 4, 2026

Related U.S. Application Data
(63) Continuation of application No. PCT/KR2023/019016, filed on Nov. 23, 2023.

(30) Foreign Application Priority Data
Jul. 6, 2023 (KR) 10-2023-0087779

(51) Int. Cl. H10K 50/11; C07B 59/00; C07D 403/04; H10K 85/60

(52) U.S. Cl. CPC H10K 50/11; C07B 59/004; C07D 403/04; H10K 85/622; H10K 85/626; H10K 85/654; H10K 85/6572; H10K 85/6574; H10K 85/6576; C07B 2200/05

(57) ABSTRACT

Provided are a heterocyclic compound of Chemical Formula 1, an organic light emitting device including the same, and a composition for forming an organic material layer of the organic light emitting device. When the heterocyclic compound of Chemical Formula 1 used in an organic light emitting device, the heterocyclic compound described in the present specification can lower the driving voltage of the device, improve the light emitting efficiency, and improve the service life characteristics of the device.

### CROSS-REFERENCED TO RELATED APPLICATION

This application is a bypass continuation application of International Patent Application No. PCT/KR2023/019016 filed on Nov. 23, 2023, which claims priority to and the benefit of Korean Patent Application No. 10-2023-0087779 filed in the Korean Intellectual Property Office on Jul. 6, 2023, the entire contents of which are incorporated herein by reference.

### TECHNICAL FIELD

The present specification relates to a heterocyclic compound, an organic light emitting device including the same, and a composition for forming an organic material layer of the organic light emitting device.

### BACKGROUND ART

An electroluminescence device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multiple layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may play a role such as a hole injection, hole transport, electron blocking, hole blocking, electron transport or electron injection.

In order to improve the performance, service life, or efficiency of the organic light emitting device, there is a continuous need for developing a material for an organic thin film.

### RELATED ART DOCUMENT

(Patent Document 1) U.S. Pat. No. 4,356,429

### DISCLOSURE

### Technical Problem

The present specification has been made in an effort to provide a heterocyclic compound, an organic light emitting device including the same, and a composition for forming an organic material layer of the organic light emitting device.

### Technical Solution

In an exemplary embodiment of the present specification, provided is a heterocyclic compound of the following Chemical Formula 1.

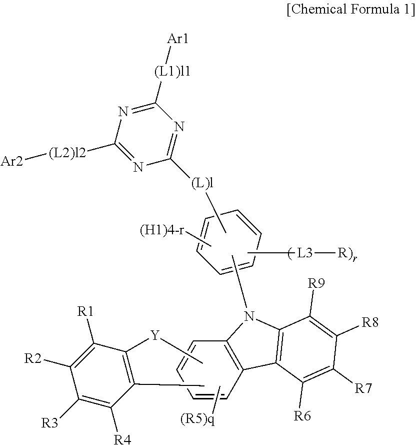

In Chemical Formula 1,
L and L1 to L3 are each independently a direct bond; a substituted or unsubstituted C6 to C60 arylene group; or a substituted or unsubstituted C2 to C60 heteroarylene group,
l, l1 and l2 are each an integer from 1 to 3, and when l, l1 and l2 are each 2 or greater, substituents in the parenthesis are the same or different,
Ar1 and Ar2 are each independently a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
R is a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
r is an integer from 1 to 4, and when r is 2 or greater, L3 and R are the same or different,
H1 is hydrogen; or deuterium, and when r is 2 or less, H1 is the same or different,
Y is O; S; C(R10)(R11) or N(R12),
R1 to R12 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, at least one of R1 to R9 is deuterium, and
q is 1 or 2, and when q is 2, R5 is the same or different.

In another exemplary embodiment of the present specification, provided is an organic light emitting device including: a first electrode; a second electrode; and an organic material layer having one or more layers provided between the first electrode and the second electrode, in which one or more layers of the organic material layer include one or more of the heterocyclic compounds.

In still another exemplary embodiment of the present specification, provided is a composition for forming an organic material layer of an organic light emitting device, including the heterocyclic compound.

### Advantageous Effects

When used in an organic light emitting device, the heterocyclic compound described in the present specification can lower the driving voltage of the device, improve the light emitting efficiency, and improve the service life characteristics of the device. Specifically, the heterocyclic compound of the present invention includes a structure in which a carbazole fused derivative substituent and a triazine group are linked by a linker including a substituted phenylene group, as shown in Chemical Formula 1, and the carbazole fused derivative substituent includes at least one deuterium, so that a linker including a substituent at the ortho position can properly separate the HOMO distributed in the fused carbazole and the LUMO electron cloud distributed in the triazine derivative, thereby forming an exciton more stable than a compound without a substituent. In addition, one or more substituted deuteriums are heavier and have lower molecular vibrational energy than light hydrogens, so that they can exhibit an effect of increasing the service life when applied in a device.

### DESCRIPTION OF DRAWINGS

FIGS. 1 to 3 are views each exemplarily illustrating a stacking structure of an organic light emitting device according to an exemplary embodiment of the present specification.

### EXPLANATION OF REFERENCE NUMERALS AND SYMBOLS

100: Substrate
200: Positive electrode
300: Organic material layer
301: Hole injection layer
302: Hole transport layer
303: Light Emitting Layer
304: Hole blocking layer
305: Electron transport layer
306: Electron injection layer
400: Negative electrode

### MODE FOR INVENTION

Hereinafter, the present specification will be described in more detail.

When one part “includes” one constituent element in the present specification, unless otherwise specifically described, this does not mean that another constituent element is excluded, but means that another constituent element may be further included.

In the present specification,

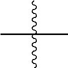

or [symbol] 0 of a chemical formula means a position to which a constituent element is bonded.

The term “substitution” means that a hydrogen atom bonded to a carbon atom or nitrogen atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means being unsubstituted or substituted with one or more substituents selected from the group consisting of deuterium; a halogen group; CN; a C1 to C60 alkyl group; a C2 to C60 alkenyl group; a C2 to C60 alkynyl group; a C1 to C60 haloalkyl group; a C1 to C60 alkoxy group; a C6 to C60 aryloxy group; a C1 to C60 alkylthioxy group; a C6 to C60 arylthioxy group; a C1 to C60 alkylsulfoxy group; a C6 to C60 arylsulfoxy group; a C3 to C60 cycloalkyl group; a C2 to C60 heterocycloalkyl group; a C6 to C60 aryl group; a C2 to C60 heteroaryl group; a silyl group; a phosphine oxide group; and an amine group, or a substituent to which two or more substituents selected among the substituents are linked, and R, R′ and R″ are each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the deuterium substitution rate may be 0% to 100%.

In the present specification, the expression of the deuterium substitution rate may be replaced with the deuterium content. That is, 100% deuterium substitution rate and 100% deuterium content are the same.

In an exemplary embodiment of the present application, in “the case where a substituent is not indicated in the structure of a chemical formula or compound”, when the deuterium substitution rate is 0%, the content of hydrogen is 100%, and all the substituents do not explicitly exclude deuterium through the definition of hydrogen and the like, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or ²H.

In an exemplary embodiment of the present application, the isotope means an atom with the same atomic number (Z), but different mass numbers (A), and the isotope may also be interpreted as an element which has the same number of protons, but different number of neutrons. In an exemplary embodiment of the present application, when the total number of substituents of a basic compound is defined as T1 and the number of specific substituents among the substituents is defined as T2, the content T % of the specific substituent may be defined as T2/T1×100=T %.

That is, in an example, a deuterium substitution rate of 20% in a phenyl group represented by

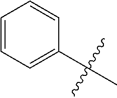 [SMILES: *c1ccccc1]

may be represented by 20% when the total number of substituents that the phenyl group can have is 5 (T1 in the formula) and the number of deuteriums among the substituents is 1 (T2 in the formula). That is, a deuterium substitution rate of 20% in the phenyl group may be represented by the following structural formula.

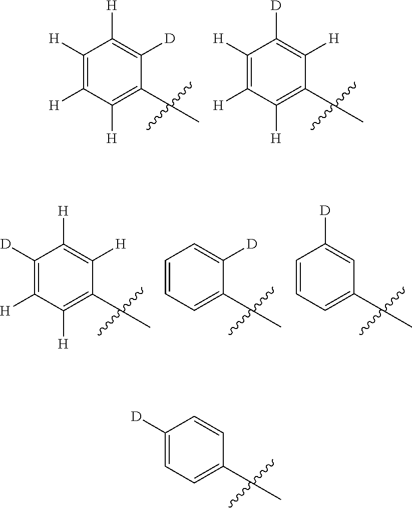 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

Further, in an exemplary embodiment of the present application, “a phenyl group having a deuterium substitution rate of 0%” may mean a phenyl group that does not include a deuterium atom, that is, has five hydrogen atoms.

In the present specification, the halogen may be fluorine, chlorine, bromine or iodine.

In the present specification, an alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, an alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl) vinyl-1-yl group, a 2,2-bis(diphenyl-1-yl) vinyl-1-yl group, a stilbenyl group, a styrenyl group, and the like, but are not limited thereto.

In the present specification, an alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, a haloalkyl group means an alkyl group substituted with a halogen group, and specific examples thereof include —CF₃, —CF₂CF₃, and the like, but are not limited thereto.

In the present specification, an alkoxy group is represented by —O(R101), and the above-described examples of the alkyl group may be applied to R101.

In the present specification, an aryloxy group is represented by —O(R102), and the above-described examples of the aryl group may be applied to R102.

In the present specification, an alkylthioxy group is represented by —S(R103), and the above-described examples of the alkyl group may be applied to R103.

In the present specification, an arylthioxy group is represented by —S(R104), and the above-described examples of the aryl group may be applied to R104.

In the present specification, an alkylsulfoxy group is represented by —S(═O)₂(R105), and the above-described examples of the alkyl group may be applied to R105.

In the present specification, an arylsulfoxy group is represented by —S(═O)₂(R106), and the above-described examples of the aryl group may be applied to R106.

In the present specification, a cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, a heterocycloalkyl group includes O, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, an aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

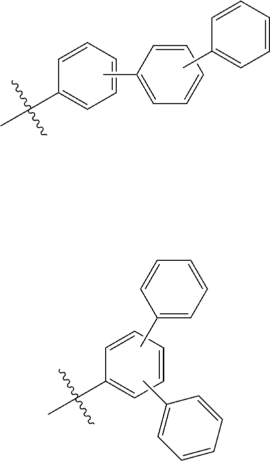

In the present specification, the fluorenyl group may be substituted, and adjacent substituents may be bonded to each other to form a ring.

When the fluorenyl group is substituted, the substituent may be the following structures, but is not limited thereto.

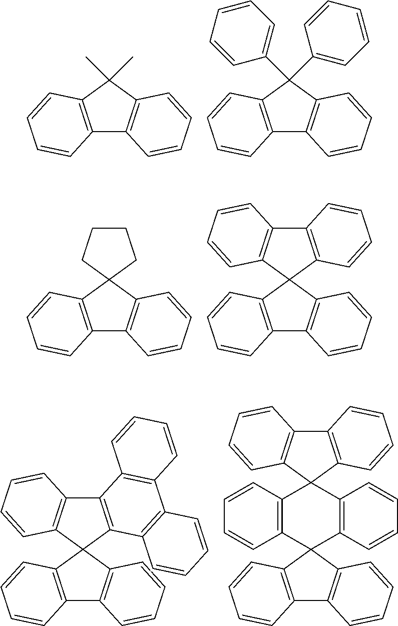

In the present specification, a heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinozoline group, a naphthyridine group, an acridine group, a phenanthridine group, an imidazopyridine group, a diazanaphthalene group, a triazaindene group, an indole group, an indolizine group, a benzothiazole group, a benzoxazole group, a benzimidazole group, a benzothiophene group, a benzofuran group, a dibenzothiophene group, a dibenzofuran group, a carbazole group, a benzocarbazole group, a dibenzocarbazole group, a phenazine group, a dibenzosilole group, spirobi (dibenzosilole), a dihydrophenazine group, a phenoxazine group, a phenanthridine group, a thienyl group, an indolo[2,3-a]carbazole group, an indolo[2,3-b]carbazole group, an indoline group, a 10,11-dihydro-dibenzo[b,f]azepine group, a 9,10-dihydroacridine group, a phenanthrazine group, a phenothiazine group, a phthalazine group, a phenanthroline group, a naphthobenzofuran group, a naphthobenzothiophene group, a benzo[c][1,2,5]thiadiazole group, a 2,3-dihydrobenzo[b]thiophene group, a 2,3-dihydrobenzofuran group, a 5,10-dihydrodibenzo[b,e][1,4]azasiline group, a pyrazolo[1,5-c]quinazoline group, a pyrido[1,2-b]indazole group, a pyrido[1,2-a]imidazo[1,2-e]indoline group, a 5,11-dihydroindeno[1,2-b]carbazole group, and the like, but are not limited thereto.

In the present specification, a benzocarbazole group may be any one of the following structures.

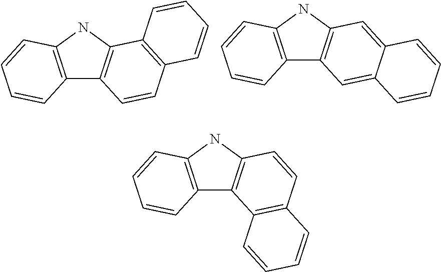

In the present specification, a dibenzocarbazole group may be any one of the following structures.

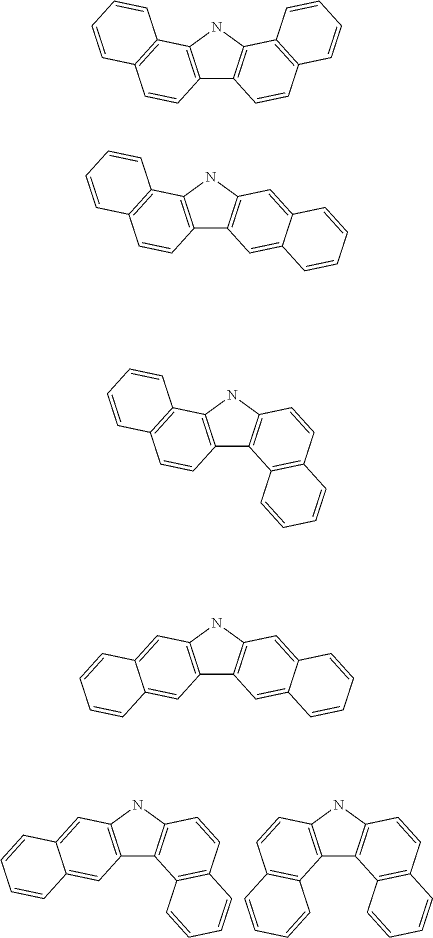

In the present specification, when the substituent is a carbazole group, a benzocarbazole group, or a dibenzocarbazole group, it means being bonded to the nitrogen or carbon of the carbazole group, the benzocarbazole group, or the dibenzocarbazole group.

In the present specification, when a carbazole group, a benzocarbazole group, or a dibenzocarbazole group is substituted, an additional substituent may be substituted at the nitrogen or carbon of the carbazole group, the benzocarbazole group, or the dibenzocarbazole group.

In the present specification, a naphthobenzofuran group may be any one of the following structures.

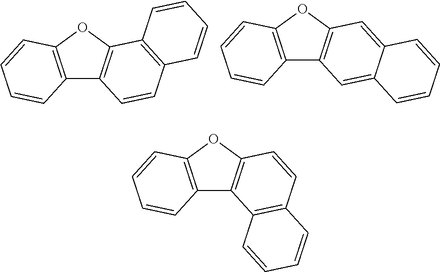

In the present specification, a naphthobenzothiophene group may be any one of the following structures.

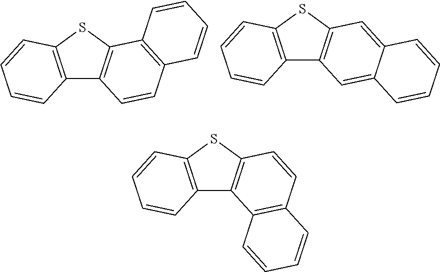

In the present specification, a silyl group includes Si and is a substituent to which the Si atom is directly linked as a radical, and is represented by —Si(R107)(R108)(R109), and R107 to R109 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specific examples of the silyl group include the following structures, but are not limited thereto.

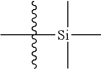 [SMILES: *[Si](C)(C)C]

(a trimethylsilyl group),

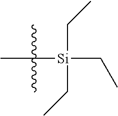 [SMILES: *[Si](CC)(CC)CC]

(a triethylsilyl group),

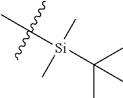 [SMILES: *[Si](C)(C)C(C)(C)C]

(a t-butyldimethylsilyl group),

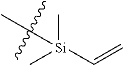 [SMILES: *[Si](C)(C)C=C]

(a vinyldimethylsilyl group),

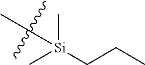 [SMILES: *[Si](C)(C)CCC]

(a propyldimethylsilyl group),

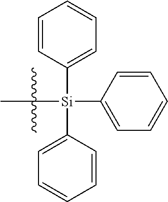 [SMILES: *[Si](c1ccccc1)(c1ccccc1)c1ccccc1]

(a triphenylsilyl group),

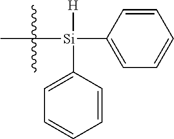 [SMILES: *[SiH](c1ccccc1)c1ccccc1]

(a diphenylsilyl group), and

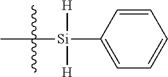 [SMILES: *[SiH2]c1ccccc1]

(a phenylsilyl group)

In the present specification, a phosphine oxide group is represented by —P(═O)(R110)(R111), and R110 and R111 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, dinaphthylphosphine oxide, and the like, but are not limited thereto.

In the present specification, an amine group is represented by —N(R112)(R113), and R112 and R113 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the above-described description of the aryl group may be applied to an arylene group except for a divalent arylene group.

In the present specification, the above-described description of the heteroaryl group may be applied to a heteroarylene group except for a divalent heteroarylene group.

An exemplary embodiment of the present specification provides the heterocyclic compound represented by Chemical Formula 1.

In an exemplary embodiment of the present specification, L and L1 to L3 of Chemical Formula 1 are each independently a direct bond; a substituted or unsubstituted C6 to C60 arylene group; or a substituted or unsubstituted C2 to C60 heteroarylene group.

In an exemplary embodiment of the present specification, L may be a direct bond; or a substituted or unsubstituted C6 to C60 arylene group.

In an exemplary embodiment of the present specification, L may be a direct bond; or a substituted or unsubstituted C6 to C40 arylene group.

In an exemplary embodiment of the present specification, L may be a direct bond; or a substituted or unsubstituted C6 to C20 arylene group.

In an exemplary embodiment of the present specification, L may be a direct bond; or a substituted or unsubstituted phenylene group.

In an exemplary embodiment of the present specification, L may be a direct bond; or a C6 to C40 arylene group unsubstituted or substituted with deuterium.

In an exemplary embodiment of the present specification, L may be a direct bond; or a C6 to C20 arylene group unsubstituted or substituted with deuterium.

In an exemplary embodiment of the present specification, L may be a direct bond; or a phenylene group unsubstituted or substituted with deuterium.

In an exemplary embodiment of the present specification, l is the number of repetitions of L and is an integer from 1 to 3, and when l is 2 or greater, two or more Ls are the same as or different from each other.

For example, when l is 2, L may be represented by -L-L′-, and L′ is the same as the definition of L.

In an exemplary embodiment of the present specification, L1 and L2 may be each independently a direct bond; a substituted or unsubstituted C6 to C40 arylene group; or a substituted or unsubstituted C2 to C40 heteroarylene group.

In an exemplary embodiment of the present specification, L1 and L2 may be each independently a direct bond; a substituted or unsubstituted C6 to C20 arylene group; or a substituted or unsubstituted C2 to C20 heteroarylene group.

In an exemplary embodiment of the present specification, L1 and L2 may be each independently a direct bond; a substituted or unsubstituted phenylene group; a substituted or unsubstituted divalent dibenzofuran group; or a substituted or unsubstituted divalent dibenzothiophene group.

In an exemplary embodiment of the present specification, L1 and L2 may be each a direct bond; or a substituted or unsubstituted C6 to C20 arylene group.

In an exemplary embodiment of the present specification, L1 and L2 may be each independently a direct bond; or a substituted or unsubstituted phenylene group.

In an exemplary embodiment of the present specification, L1 and L2 may be each independently a direct bond; a C6 to C20 arylene group; or a C2 to C20 heteroarylene group.

In an exemplary embodiment of the present specification, L1 and L2 may be each independently a direct bond; or a C6 to C20 arylene group.

In an exemplary embodiment of the present specification, l1 is the number of repetitions of L1 and is an integer from 1 to 3, and when l1 is 2 or greater, two or more L1s are the same as or different from each other.

For example, when l1 is 2, L1 may be represented by -L1-L1′-, and L1′ is the same as the definition of L1.

In an exemplary embodiment of the present specification, l2 is the number of repetitions of L2 and is an integer from 1 to 3, and when l2 is 2 or greater, two or more L2s are the same as or different from each other.

For example, when l2 is 2, L2 may be represented by -L2-L2′-, and L2′ is the same as the definition of L2.

In an exemplary embodiment of the present specification, L3 may be a direct bond; a substituted or unsubstituted C6 to C40 arylene group; or a substituted or unsubstituted C2 to C40 heteroarylene group.

In an exemplary embodiment of the present specification, L3 may be a direct bond; a substituted or unsubstituted C6 to C20 arylene group; or a substituted or unsubstituted C2 to C20 heteroarylene group.

In an exemplary embodiment of the present specification, L3 may be a direct bond; a substituted or unsubstituted phenylene group; a substituted or unsubstituted divalent dibenzofuran group; or a substituted or unsubstituted divalent dibenzothiophene group.

In an exemplary embodiment of the present specification, L3 may be a direct bond; a C6 to C20 arylene group unsubstituted or substituted with deuterium; or a C2 to C20 heteroarylene group unsubstituted or substituted with deuterium.

In an exemplary embodiment of the present specification, Ar1 and Ar2 of Chemical Formula 1 are each independently a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In an exemplary embodiment of the present specification, Ar1 and Ar2 may be each independently a substituted or unsubstituted C6 to C60 aryl group; or a substituted or unsubstituted C2 to C60 heteroaryl group.

In an exemplary embodiment of the present specification, Ar1 and Ar2 may be each independently a substituted or unsubstituted C6 to C40 aryl group; or a substituted or unsubstituted C2 to C40 heteroaryl group.

In an exemplary embodiment of the present specification, Ar1 and Ar2 may be each independently a substituted or unsubstituted C6 to C20 aryl group; or a substituted or unsubstituted C2 to C20 heteroaryl group.

In an exemplary embodiment of the present specification, Ar1 and Ar2 may be each independently a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; a substituted or unsubstituted triphenylene group; a substituted or unsubstituted dibenzofuran group; a substituted or unsubstituted dibenzothiophene group; or a substituted or unsubstituted carbazole group.

In an exemplary embodiment of the present specification, Ar1 and Ar2 may be each independently a C6 to C20 aryl group unsubstituted or substituted with an alkyl group; or a C2 to C20 heteroaryl group unsubstituted or substituted with an aryl group.

In an exemplary embodiment of the present specification, Ar1 and Ar2 may be each independently a phenyl group; a biphenyl group; a terphenyl group; a naphthyl group; a dimethylfluorenyl group; a triphenylene group; a dibenzofuran group unsubstituted or substituted with a phenyl group; a dibenzothiophene group unsubstituted or substituted with a phenyl group; or a carbazole group unsubstituted or substituted with a phenyl group.

In an exemplary embodiment of the present specification, Ar1 may be a phenyl group; a biphenyl group; a naphthyl group; or a carbazole group unsubstituted or substituted with a phenyl group.

In an exemplary embodiment of the present specification, Ar1 may be a substituted or unsubstituted phenyl group; a substituted or unsubstituted biphenyl group; a substituted or unsubstituted naphthyl group; or a substituted or unsubstituted carbazole group.

In an exemplary embodiment of the present specification, Ar2 may be a substituted or unsubstituted phenyl group; a substituted or unsubstituted biphenyl group; a substituted or unsubstituted terphenyl group; a substituted or unsubstituted naphthyl group; a substituted or unsubstituted dimethylfluorenyl group; a substituted or unsubstituted triphenylene group; a substituted or unsubstituted dibenzofuran group; a substituted or unsubstituted dibenzothiophene group; or a substituted or unsubstituted carbazole group.

In an exemplary embodiment of the present specification, Ar2 may be a phenyl group; a biphenyl group; a terphenyl group; a naphthyl group; a dimethylfluorenyl group; a triphenylene group; a dibenzofuran group unsubstituted or substituted with a phenyl group; a dibenzothiophene group unsubstituted or substituted with a phenyl group; or a carbazole group unsubstituted or substituted with a phenyl group.

In an exemplary embodiment of the present specification, R of Chemical Formula 1 is a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In an exemplary embodiment of the present specification, R may be a substituted or unsubstituted C6 to C60 aryl group; or a substituted or unsubstituted C2 to C60 heteroaryl group.

In an exemplary embodiment of the present specification, R may be a substituted or unsubstituted C6 to C40 aryl group; or a substituted or unsubstituted C2 to C40 heteroaryl group.

In an exemplary embodiment of the present specification, R may be a substituted or unsubstituted C6 to C20 aryl group; or a substituted or unsubstituted C2 to C20 heteroaryl group.

In an exemplary embodiment of the present specification, R may be a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; a substituted or unsubstituted triphenylene group; a substituted or unsubstituted dibenzofuran group; a substituted or unsubstituted dibenzothiophene group; or a substituted or unsubstituted carbazole group.

In an exemplary embodiment of the present specification, R may be a C6 to C20 aryl group unsubstituted or substituted with one or more substituents of deuterium, an alkyl group and a heteroaryl group; or a C2 to C20 heteroaryl group unsubstituted or substituted with one or more substituents of deuterium and an aryl group.

In an exemplary embodiment of the present specification, R may be a phenyl group unsubstituted or substituted with deuterium; a biphenyl group unsubstituted or substituted with deuterium; a terphenyl group unsubstituted or substituted with deuterium; a naphthyl group unsubstituted or substituted with deuterium; a fluorenyl group unsubstituted or substituted with one or more substituents of deuterium and an alkyl group; a triphenylene group unsubstituted or substituted with deuterium; a dibenzofuran group unsubstituted or substituted with one or more substituents of deuterium and an aryl group; a dibenzothiophene group unsubstituted or substituted with one or more substituents of deuterium and an aryl group; or a carbazole group unsubstituted or substituted with one or more substituents of deuterium and an aryl group.

In an exemplary embodiment of the present specification, r is the number of -L3-Rs to be substituted with a benzene ring and is an integer from 1 to 4, and when r is 2 or greater, two or more L3s and Rs are each the same or different.

For example, when r is 2, the benzene ring is substituted with two substituents represented by -L3-R and -L3′-R′, L3′ is the same as the definition of L3, and R′ is the same as the definition of R.

In an exemplary embodiment of the present specification, r may be 1.

In an exemplary embodiment of the present specification, H1 of Chemical Formula 1 is hydrogen; or deuterium.

In an exemplary embodiment of the present specification, 4−r is the number of —H1s to be substituted with a benzene ring, and when r is 2 or less, two or more H1s are each the same or different.

In an exemplary embodiment of the present specification, the sum of the number of the substituent -L3-Rs and the number of the substituent —H1s is 4.

In an exemplary embodiment of the present specification, Y of Chemical Formula 1 is O; S; C(R10)(R11) or N(R12), and R10 to R12 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In an exemplary embodiment of the present specification, Y may be O.

In an exemplary embodiment of the present specification, Y may be S.

In an exemplary embodiment of the present specification, Y is C(R10)(R11), and R10 and R11 may be each independently a substituted or unsubstituted C1 to C10 alkyl group.

In an exemplary embodiment of the present specification, Y is C(R10)(R11), and R10 and R11 may be each independently a substituted or unsubstituted methyl group.

In an exemplary embodiment of the present specification, Y is N(R12), and R12 may be a substituted or unsubstituted C6 to C20 aryl group.

In an exemplary embodiment of the present specification, Y is N(R12), and R12 may be a substituted or unsubstituted phenyl group; or a substituted or unsubstituted biphenyl group.

In an exemplary embodiment of the present specification, Y is N(R12), and R12 may be a C6 to C20 aryl group unsubstituted or substituted with deuterium.

In an exemplary embodiment of the present specification, Y is N(R12), and R12 may be a phenyl group unsubstituted or substituted with deuterium; or a biphenyl group unsubstituted or substituted with deuterium.

In an exemplary embodiment of the present specification, Chemical Formula 1 may be represented by any one of the following Chemical Formulae 1-1 to 1-3.

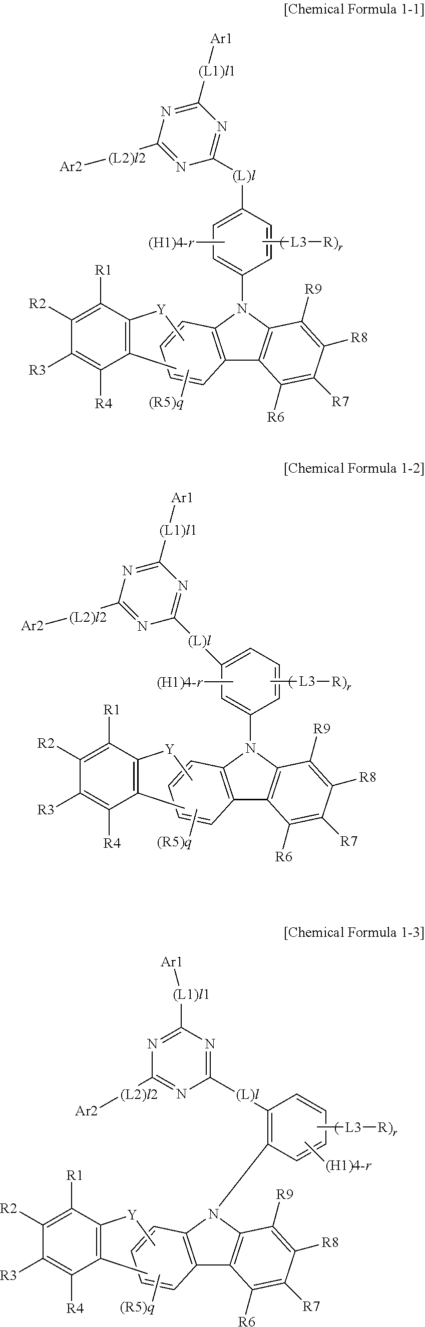

In Chemical Formulae 1-1 to 1-3,
the definition of each substituent is the same as the definition in Chemical Formula 1.

In an exemplary embodiment of the present specification, Chemical Formula 1 may be represented by any one of the following Chemical Formulae 1-4 to 1-9.

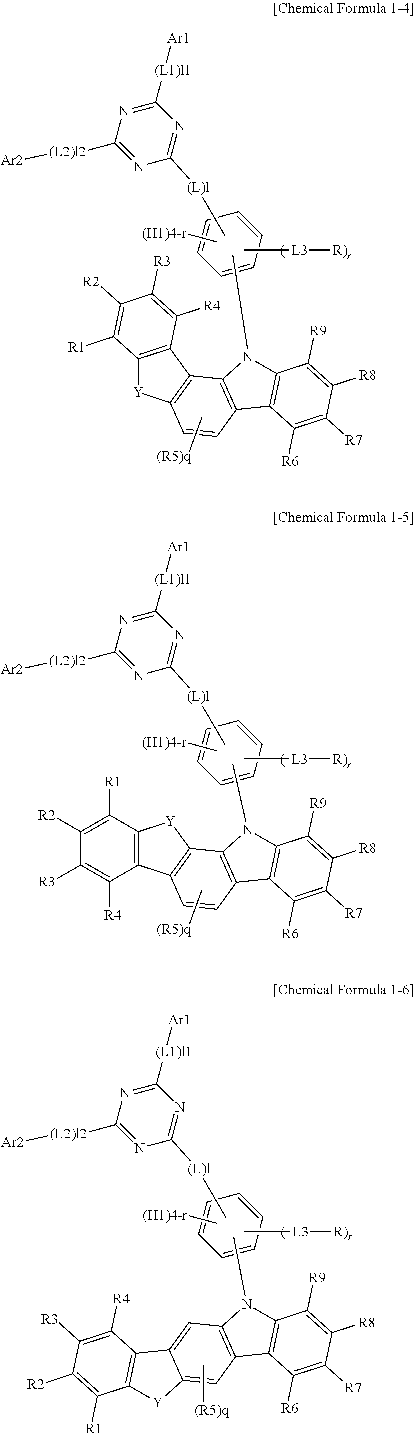

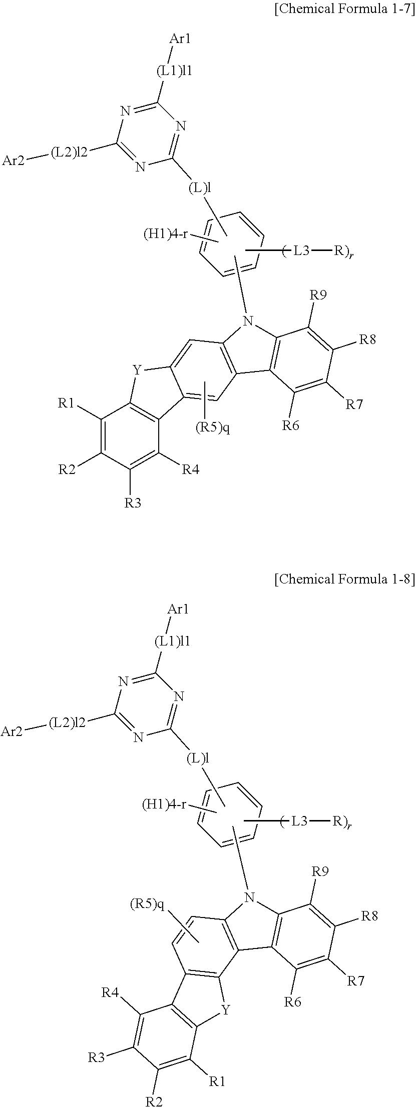

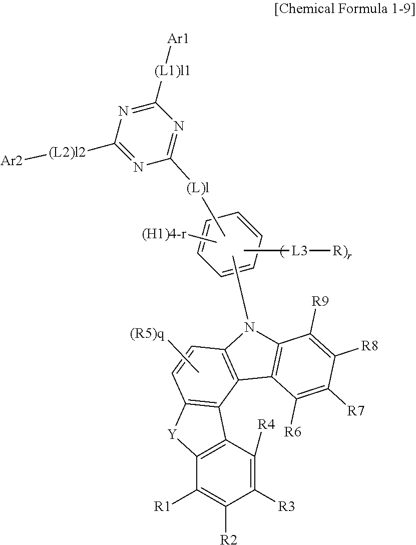

In Chemical Formulae 1-4 to 1-9,
the definition of each substituent is the same as the definition in Chemical Formula 1.

In an exemplary embodiment of the present specification, R1 to R9 of Chemical Formula 1 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and at least one of R1 to R9 is deuterium.

In an exemplary embodiment of the present specification, R1 to R9 are each independently hydrogen; deuterium; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and at least one of R1 to R9 is deuterium.

In an exemplary embodiment of the present specification, R1 to R9 may be each independently hydrogen; deuterium; a substituted or unsubstituted C1 to C60 alkyl group; a substituted or unsubstituted C6 to C60 aryl group; or a substituted or unsubstituted C2 to C60 heteroaryl group, and at least one of R1 to R9 is deuterium.

In an exemplary embodiment of the present specification, R1 to R9 may be each independently hydrogen; deuterium; a substituted or unsubstituted C1 to C10 alkyl group; a substituted or unsubstituted C6 to C20 aryl group; or a substituted or unsubstituted C2 to C20 heteroaryl group, and at least one of R1 to R9 is deuterium.

In an exemplary embodiment of the present specification, R1 to R9 may be each independently hydrogen; deuterium; or a substituted or unsubstituted C6 to C20 aryl group, and at least one of R1 to R9 is deuterium.

In an exemplary embodiment of the present specification, R1 to R9 may be each independently hydrogen; deuterium; or a substituted or unsubstituted phenyl group, and at least one of R1 to R9 is deuterium.

In an exemplary embodiment of the present specification, R1 to R9 may be each independently hydrogen; deuterium; or a C6 to C20 aryl group unsubstituted or substituted with deuterium, and at least one of R1 to R9 is deuterium.

In an exemplary embodiment of the present specification, R1 to R9 may be each independently hydrogen; deuterium; or a phenyl group unsubstituted or substituted with deuterium, and at least one of R1 to R9 is deuterium.

In an exemplary embodiment of the present specification, R1 to R9 may be each independently hydrogen; or deuterium, and at least one of R1 to R9 is deuterium.

In an exemplary embodiment of the present specification, q is the number of the substituent R5s and is 1 or 2, and when q is 2, two R5s are the same or different.

For example, when q is 2, a benzene ring with an additional fused ring in carbazole is substituted with a substituent R5 and a substituent R5′, and the definition of R5′ is the same as the definition of R5.

In an exemplary embodiment of the present specification, the deuterium substitution rate of Chemical Formula 1 may be more than 0% and 100% or less.

The deuterium substitution rate of a heterocyclic compound according to an exemplary embodiment of the present specification means the deuterium substitution rate of the total number of hydrogen and deuterium included in the compound.

In an exemplary embodiment of the present specification, the deuterium substitution rate of Chemical Formula 1 may be 10% to 100%.

In an exemplary embodiment of the present specification, the deuterium substitution rate of Chemical Formula 1 may be 30% to 100%.

In an exemplary embodiment of the present specification, the deuterium substitution rate of Chemical Formula 1 may be 50% to 100%.

In an exemplary embodiment of the present specification, the deuterium substitution rate of Chemical Formula 1 may be 70% to 100%.

In an exemplary embodiment of the present specification, the deuterium substitution rate of Chemical Formula 1 may be more than 0% and less than 100%.

In an exemplary embodiment of the present specification, the deuterium substitution rate of Chemical Formula 1 may be 10% to 99%.

In an exemplary embodiment of the present specification, the deuterium substitution rate of Chemical Formula 1 may be 30% to 90%.

In an exemplary embodiment of the present specification, the deuterium substitution rate of Chemical Formula 1 may be 50% to 90%.

In an exemplary embodiment of the present specification, the deuterium substitution rate of Chemical Formula 1 may be 70% to 90%.

In an exemplary embodiment of the present specification, the deuterium content of the heterocyclic compound of Chemical Formula 1 satisfies the above range, the photochemical characteristics of a compound which includes deuterium and a compound which does not include deuterium are almost similar, but when deposited on a thin film, the deuterium-containing material tends to be packed with a narrower intermolecular distance.

Accordingly, when an electron only device (EOD) and a hole only device (HOD) are manufactured and the current density thereof according to voltage is confirmed, it can be confirmed that the heterocyclic compound of Chemical Formula 1 of the present invention exhibits much more balanced charge transport characteristics than a compound which has the same structure and does not include deuterium.

Further, when the surface of a thin film is observed using an atomic force microscope (AFM), it can be confirmed that the thin film made of a compound including deuterium is deposited with a more uniform surface without any aggregated portion.

Additionally, since the single bond dissociation energy of carbon and deuterium is higher than the single bond dissociation energy of carbon and hydrogen, the stability of the total molecules of the heterocyclic compound of Chemical Formula 1 of the present invention is enhanced, so that there is an effect that the service life of the device is improved.

In an exemplary embodiment of the present specification, Chemical Formula 1 is represented by [Structure A]-[Structure B]-[Structure C], Structure A is represented by the following Chemical Formula A, Structure B is represented by the following Chemical Formula B, and Structure C may be represented by the following Chemical Formula C.

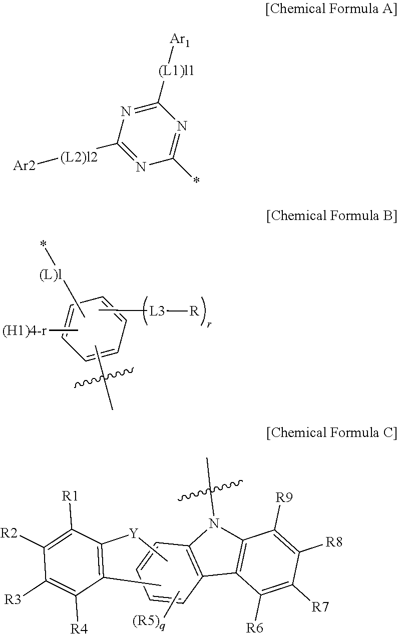

In Chemical Formulae A to C,
the definition of each substituent is the same as the definition in Chemical Formula 1,
[symbol] of Chemical Formula A is bonded to [symbol] of Chemical Formula B, and

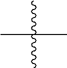

of Chemical Formula B is bonded to

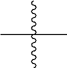

of Chemical Formula C.

In an exemplary embodiment of the present specification, the deuterium substitution rate of Structure A may be 0% to 100%.

The deuterium substitution rate of Structure A means the deuterium substitution rate of the total number of hydrogen and deuterium included in Chemical Formula A.

In an exemplary embodiment of the present specification, the deuterium substitution rate of Structure A may be 0%. That is, Structure A may not include deuterium.

In an exemplary embodiment of the present specification, the deuterium substitution rate of Structure B may be 0% to 100%.

The deuterium substitution rate of Structure B means the deuterium substitution rate of the total number of hydrogen and deuterium included in Chemical Formula B.

In an exemplary embodiment of the present specification, the deuterium substitution rate of Structure B is 0%, or may be more than 0% and 100% or less. That is, Structure does not include deuterium, or may include at least one deuterium.

In an exemplary embodiment of the present specification, the deuterium substitution rate of Structure C is more than 0% and 100% or less.

The deuterium substitution rate of Structure C means the deuterium substitution rate of the total number of hydrogen and deuterium included in Chemical Formula C.

In an exemplary embodiment of the present specification, the deuterium substitution rate of Structure C may be 10% to 100%.

In an exemplary embodiment of the present specification, the deuterium substitution rate of Structure C may be 30% to 100%.

In an exemplary embodiment of the present specification, the deuterium substitution rate of Structure C may be 50% to 100%.

In an exemplary embodiment of the present specification, the deuterium substitution rate of Structure C may be 70% to 100%.

In an exemplary embodiment of the present specification, the deuterium substitution rate of Structure C may be 80% to 100%.

In an exemplary embodiment of the present specification, the deuterium substitution rate of Structure A is 0%, the deuterium substitution rate of Structure B is 0% to 100%, and the deuterium substitution rate of Structure C may be more than 0% and 100% or less.

In an exemplary embodiment of the present specification, the deuterium substitution rate of Structure A is 0%, the deuterium substitution rate of Structure B is 0% to 100%, and the deuterium substitution rate of Structure C may be 50% to 100%.

In an exemplary embodiment of the present specification, Chemical Formula 1 may be represented by any one of the following compounds.

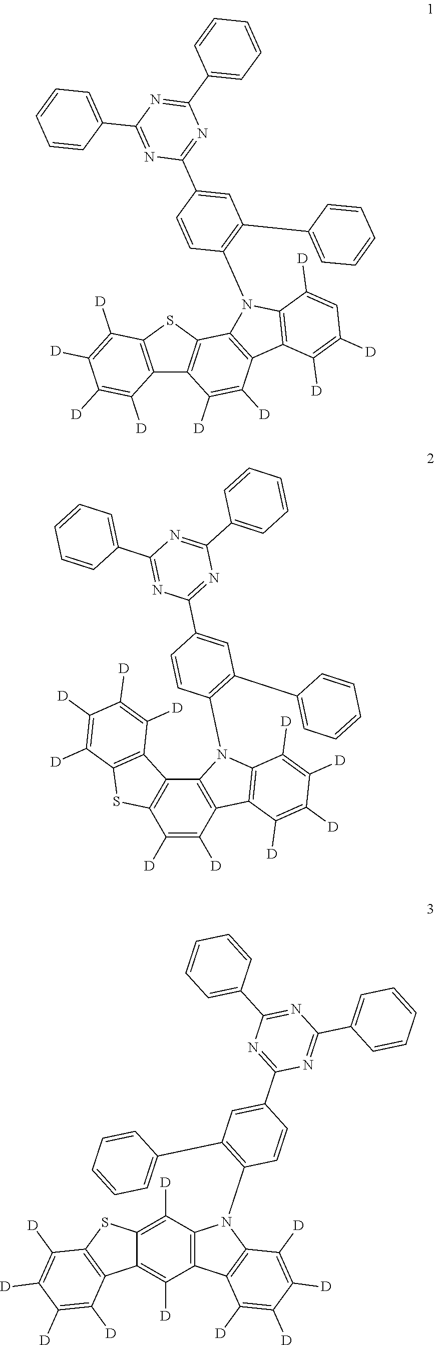 [SMILES: [2H]c1cc([2H])c2c(c1[2H])c1c([2H])c([2H])c3c(sc4c([2H])c([2H])c([2H])c([2H])c43)c1n2-c1ccc(-c2nc(-c3ccccc3)nc(-c3ccccc3)n2)cc1-c1ccccc1 ; [2H]c1c([2H])c([2H])c2c(sc3c([2H])c([2H])c4c5c([2H])c([2H])c([2H])c([2H])c5n(-c5ccc(-c6nc(-c7ccccc7)nc(-c7ccccc7)n6)cc5-c5ccccc5)c4c32)c1[2H] ; [2H]c1c([2H])c([2H])c2c(sc3c([2H])c4c(c([2H])c32)c2c([2H])c([2H])c([2H])c([2H])c2n4-c2ccc(-c3nc(-c4ccccc4)nc(-c4ccccc4)n3)cc2-c2ccccc2)c1[2H]]

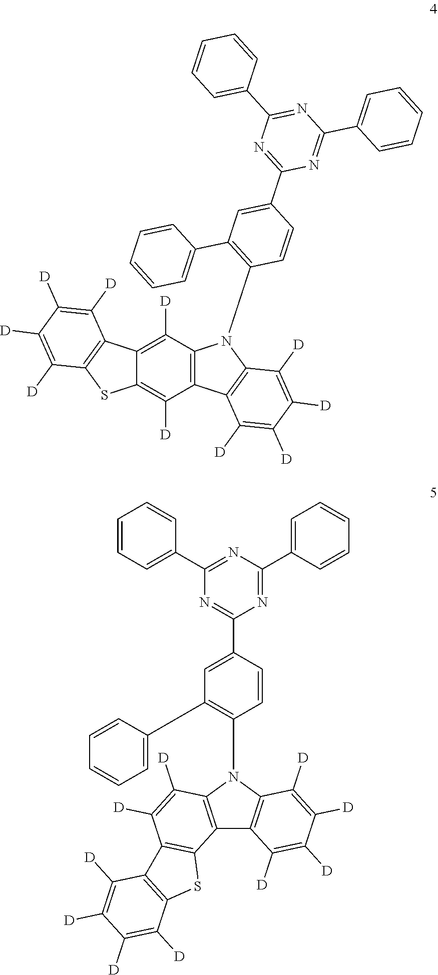 [SMILES: [2H]c1c([2H])c([2H])c2c(sc3c([2H])c4c5c([2H])c([2H])c([2H])c([2H])c5n(-c5ccc(-c6nc(-c7ccccc7)nc(-c7ccccc7)n6)cc5-c5ccccc5)c4c([2H])c32)c1[2H] ; [2H]c1c([2H])c([2H])c2c(sc3c2c([2H])c([2H])c2c3c3c([2H])c([2H])c([2H])c([2H])c3n2-c2ccc(-c3nc(-c4ccccc4)nc(-c4ccccc4)n3)cc2-c2ccccc2)c1[2H]]

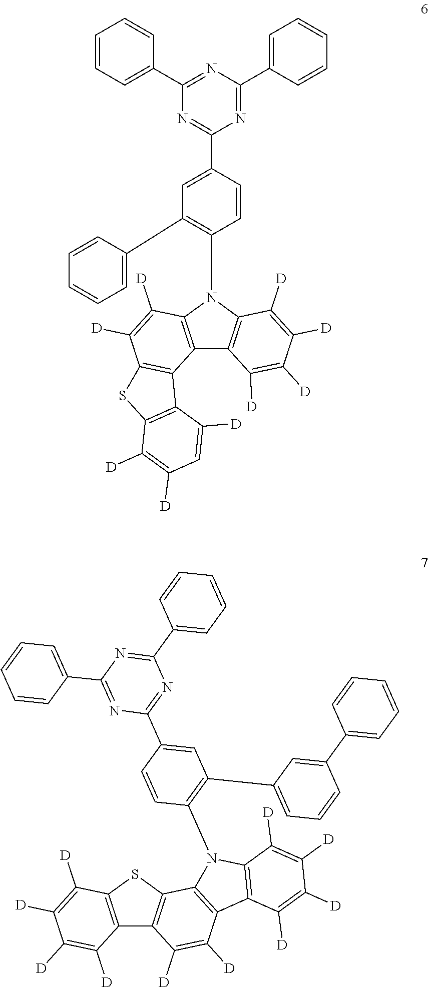 [SMILES: [2H]c1cc([2H])c2c(sc3c([2H])c([2H])c4c(c5c([2H])c([2H])c([2H])c([2H])c5n4-c4ccc(-c5nc(-c6ccccc6)nc(-c6ccccc6)n5)cc4-c4ccccc4)c32)c1[2H] ; [2H]c1c([2H])c([2H])c2c(sc3c2c([2H])c([2H])c2c4c([2H])c([2H])c([2H])c([2H])c4n(-c4ccc(-c5nc(-c6ccccc6)nc(-c6ccccc6)n5)cc4-c4cccc(-c5ccccc5)c4)c32)c1[2H]]

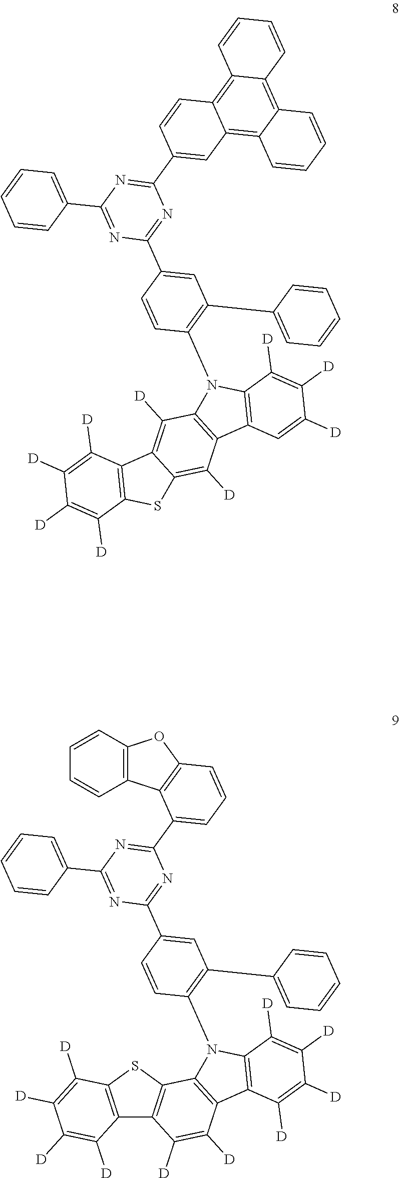 [SMILES: [2H]c1cc2c3c([2H])c4sc5c([2H])c([2H])c([2H])c([2H])c5c4c([2H])c3n(-c3ccc(-c4nc(-c5ccccc5)nc(-c5ccc6c7ccccc7c7ccccc7c6c5)n4)cc3-c3ccccc3)c2c([2H])c1[2H] ; [2H]c1c([2H])c([2H])c2c(sc3c2c([2H])c([2H])c2c4c([2H])c([2H])c([2H])c([2H])c4n(-c4ccc(-c5nc(-c6ccccc6)nc(-c6cccc7oc8ccccc8c67)n5)cc4-c4ccccc4)c32)c1[2H]]

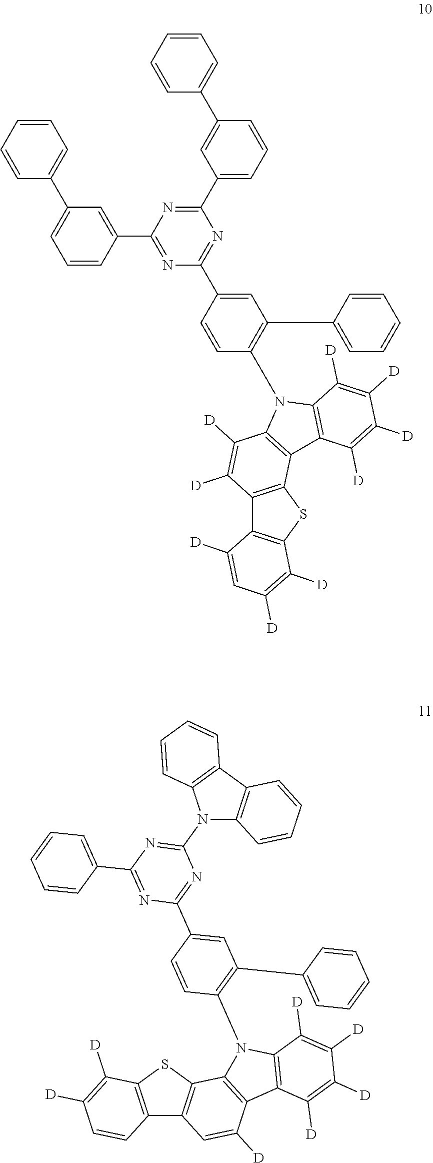 [SMILES: [2H]c1cc([2H])c2c(sc3c2c([2H])c([2H])c2c3c3c([2H])c([2H])c([2H])c([2H])c3n2-c2ccc(-c3nc(-c4cccc(-c5ccccc5)c4)nc(-c4cccc(-c5ccccc5)c4)n3)cc2-c2ccccc2)c1[2H] ; [2H]c1ccc2c(sc3c2cc([2H])c2c4c([2H])c([2H])c([2H])c([2H])c4n(-c4ccc(-c5nc(-c6ccccc6)nc(-n6c7ccccc7c7ccccc76)n5)cc4-c4ccccc4)c32)c1[2H]]

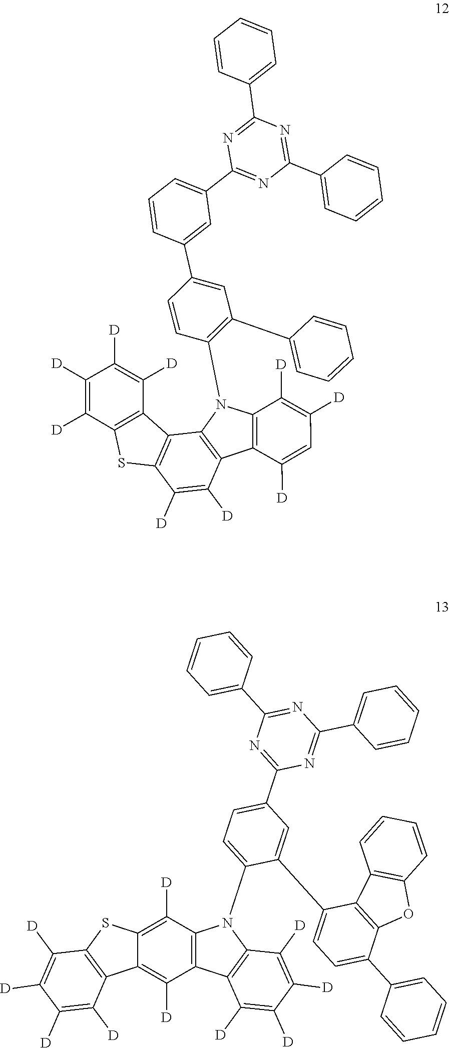 [SMILES: [2H]c1cc([2H])c2c3c([2H])c([2H])c4sc5c([2H])c([2H])c([2H])c([2H])c5c4c3n(-c3ccc(-c4cccc(-c5nc(-c6ccccc6)nc(-c6ccccc6)n5)c4)cc3-c3ccccc3)c2c1[2H] ; [2H]c1c([2H])c([2H])c2c(sc3c([2H])c4c(c([2H])c32)c2c([2H])c([2H])c([2H])c([2H])c2n4-c2ccc(-c3nc(-c4ccccc4)nc(-c4ccccc4)n3)cc2-c2ccc(-c3ccccc3)c3oc4ccccc4c23)c1[2H]]

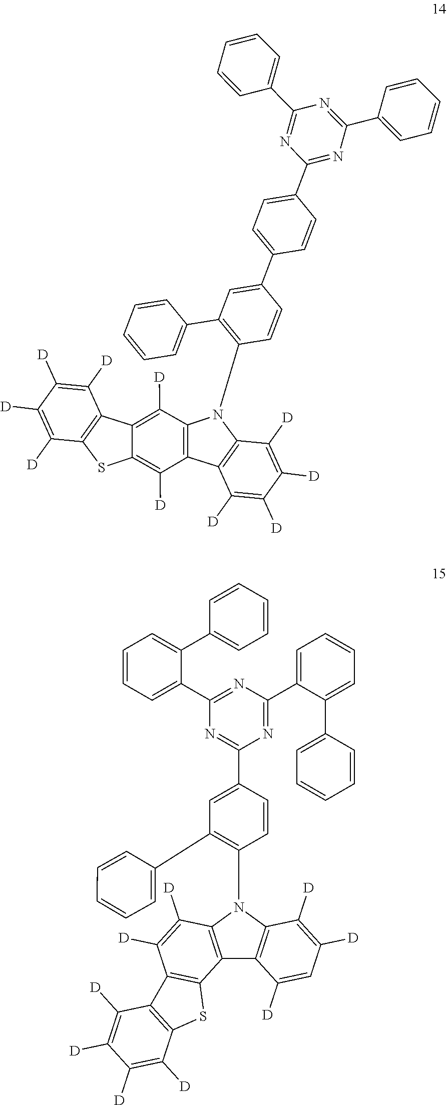 [SMILES: [2H]c1c([2H])c([2H])c2c(sc3c([2H])c4c5c([2H])c([2H])c([2H])c([2H])c5n(-c5ccc(-c6ccc(-c7nc(-c8ccccc8)nc(-c8ccccc8)n7)cc6)cc5-c5ccccc5)c4c([2H])c32)c1[2H] ; [2H]c1cc([2H])c2c3c4sc5c([2H])c([2H])c([2H])c([2H])c5c4c([2H])c([2H])c3n(-c3ccc(-c4nc(-c5ccccc5-c5ccccc5)nc(-c5ccccc5-c5ccccc5)n4)cc3-c3ccccc3)c2c1[2H]]

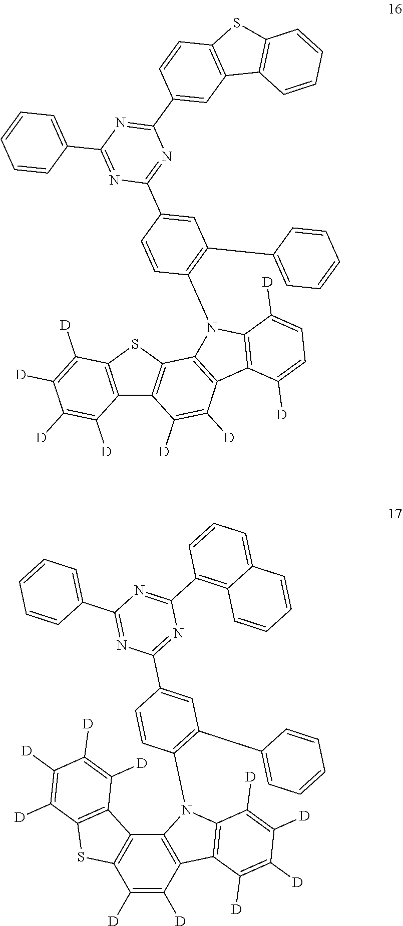 [SMILES: [2H]c1c([2H])c([2H])c2c(sc3c2c([2H])c([2H])c2c4c([2H])ccc([2H])c4n(-c4ccc(-c5nc(-c6ccccc6)nc(-c6ccc7sc8ccccc8c7c6)n5)cc4-c4ccccc4)c32)c1[2H] ; [2H]c1c([2H])c([2H])c2c(sc3c([2H])c([2H])c4c5c([2H])c([2H])c([2H])c([2H])c5n(-c5ccc(-c6nc(-c7ccccc7)nc(-c7cccc8ccccc78)n6)cc5-c5ccccc5)c4c32)c1[2H]]

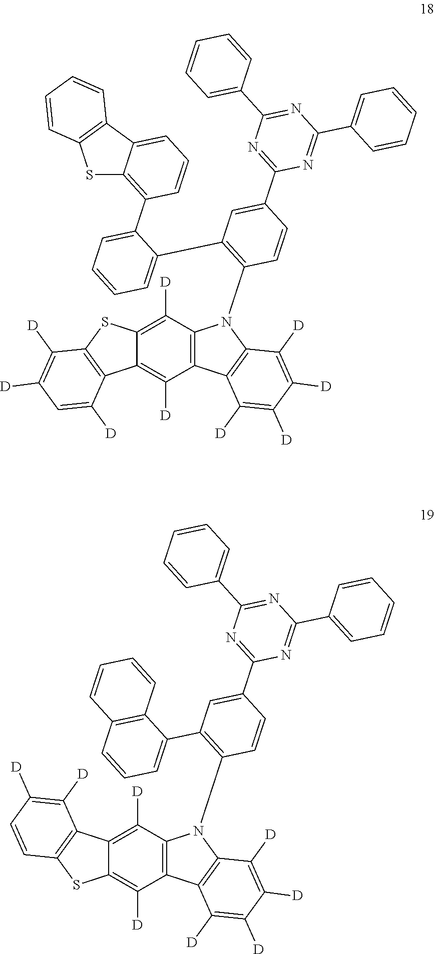 [SMILES: [2H]c1cc([2H])c2c(sc3c([2H])c4c(c([2H])c32)c2c([2H])c([2H])c([2H])c([2H])c2n4-c2ccc(-c3nc(-c4ccccc4)nc(-c4ccccc4)n3)cc2-c2ccccc2-c2cccc3c2sc2ccccc23)c1[2H] ; [2H]c1ccc2sc3c([2H])c4c5c([2H])c([2H])c([2H])c([2H])c5n(-c5ccc(-c6nc(-c7ccccc7)nc(-c7ccccc7)n6)cc5-c5cccc6ccccc56)c4c([2H])c3c2c1[2H]]

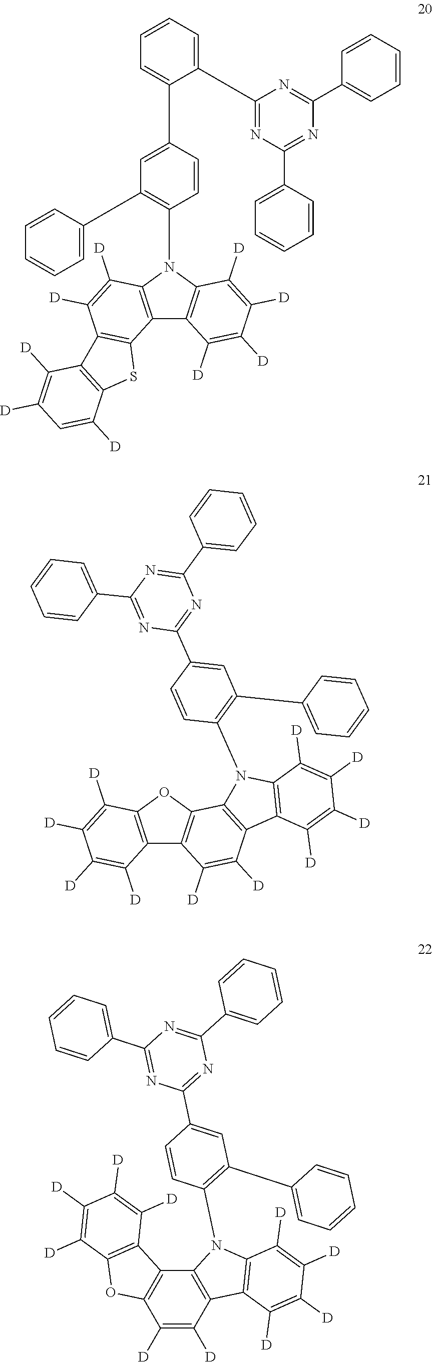 [SMILES: [2H]c1cc([2H])c2sc3c(c([2H])c([2H])c4c3c3c([2H])c([2H])c([2H])c([2H])c3n4-c3ccc(-c4ccccc4-c4nc(-c5ccccc5)nc(-c5ccccc5)n4)cc3-c3ccccc3)c2c1[2H] ; [2H]c1c([2H])c([2H])c2c(oc3c2c([2H])c([2H])c2c4c([2H])c([2H])c([2H])c([2H])c4n(-c4ccc(-c5nc(-c6ccccc6)nc(-c6ccccc6)n5)cc4-c4ccccc4)c32)c1[2H] ; [2H]c1c([2H])c([2H])c2c(oc3c([2H])c([2H])c4c5c([2H])c([2H])c([2H])c([2H])c5n(-c5ccc(-c6nc(-c7ccccc7)nc(-c7ccccc7)n6)cc5-c5ccccc5)c4c32)c1[2H]]

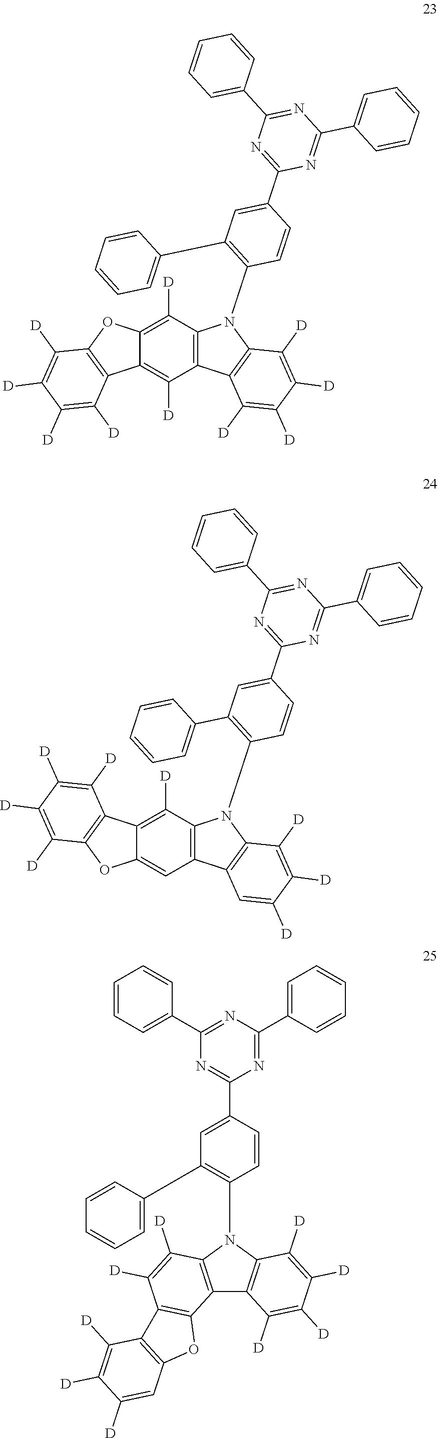

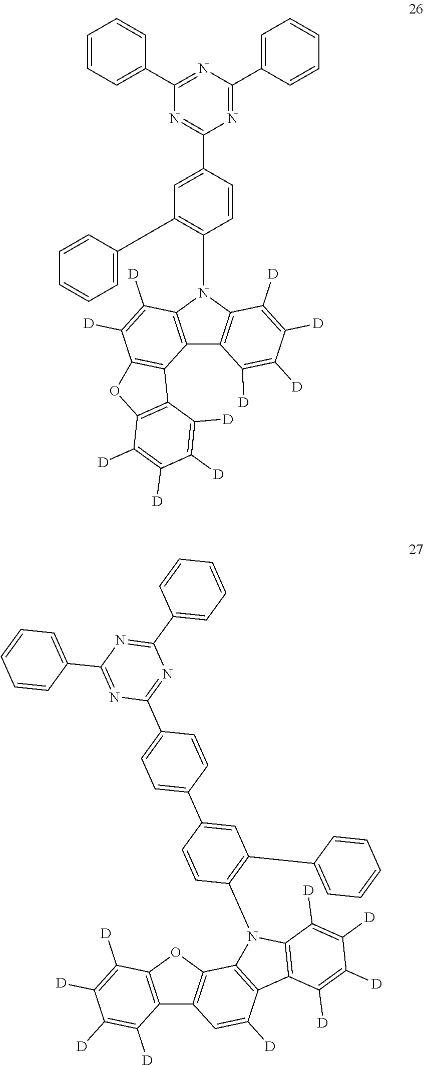 [SMILES: [2H]c1c([2H])c([2H])c2c(oc3c([2H])c([2H])c4c(c5c([2H])c([2H])c([2H])c([2H])c5n4-c4ccc(-c5nc(-c6ccccc6)nc(-c6ccccc6)n5)cc4-c4ccccc4)c32)c1[2H] ; [2H]c1c([2H])c([2H])c2c(oc3c2cc([2H])c2c4c([2H])c([2H])c([2H])c([2H])c4n(-c4ccc(-c5ccc(-c6nc(-c7ccccc7)nc(-c7ccccc7)n6)cc5)cc4-c4ccccc4)c32)c1[2H]]

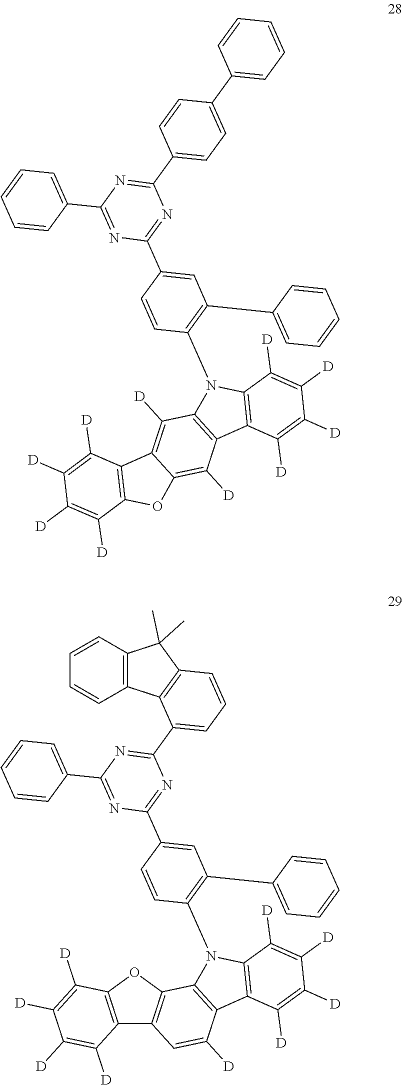 [SMILES: [2H]c1c([2H])c([2H])c2c(oc3c([2H])c4c5c([2H])c([2H])c([2H])c([2H])c5n(-c5ccc(-c6nc(-c7ccccc7)nc(-c7ccc(-c8ccccc8)cc7)n6)cc5-c5ccccc5)c4c([2H])c32)c1[2H] ; [2H]c1c([2H])c([2H])c2c(oc3c2cc([2H])c2c4c([2H])c([2H])c([2H])c([2H])c4n(-c4ccc(-c5nc(-c6ccccc6)nc(-c6cccc7c6-c6ccccc6C7(C)C)n5)cc4-c4ccccc4)c32)c1[2H]]

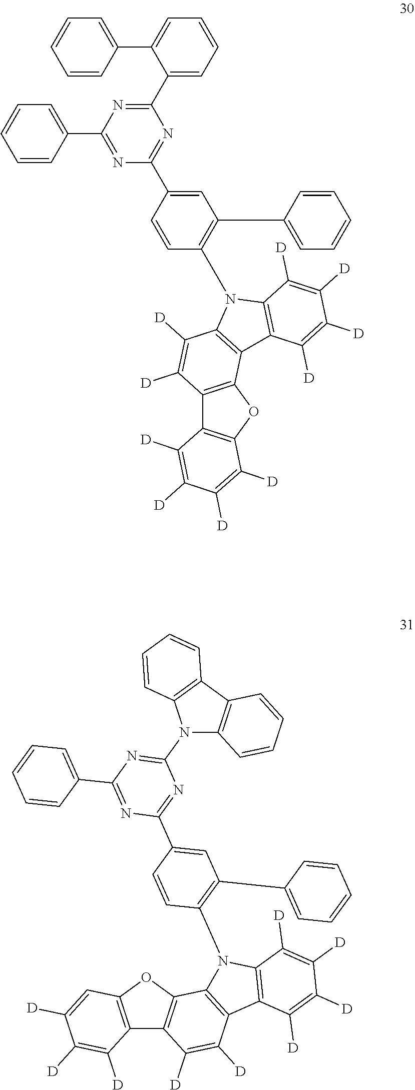 [SMILES: [2H]c1c([2H])c([2H])c2c(oc3c2c([2H])c([2H])c2c3c3c([2H])c([2H])c([2H])c([2H])c3n2-c2ccc(-c3nc(-c4ccccc4)nc(-c4ccccc4-c4ccccc4)n3)cc2-c2ccccc2)c1[2H] ; [2H]c1cc2oc3c(c([2H])c([2H])c4c5c([2H])c([2H])c([2H])c([2H])c5n(-c5ccc(-c6nc(-c7ccccc7)nc(-n7c8ccccc8c8ccccc87)n6)cc5-c5ccccc5)c34)c2c([2H])c1[2H]]

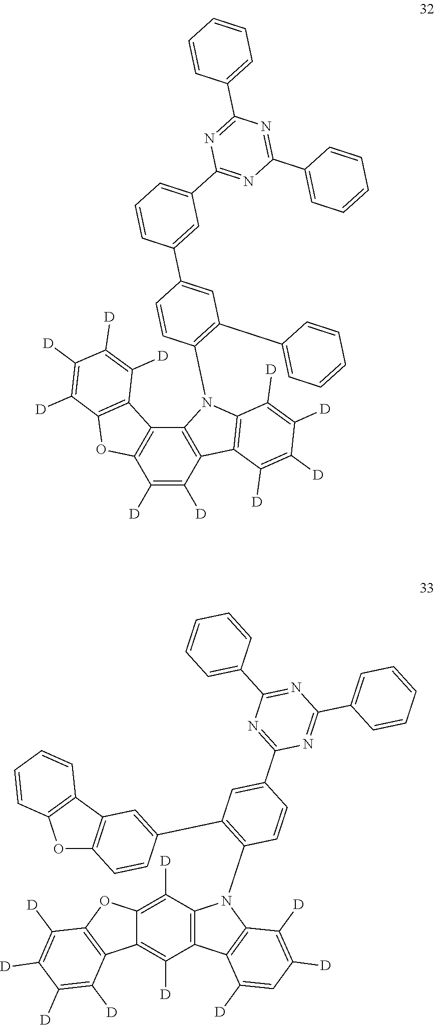 [SMILES: [2H]c1c([2H])c([2H])c2c(oc3c([2H])c([2H])c4c5c([2H])c([2H])c([2H])c([2H])c5n(-c5ccc(-c6cccc(-c7nc(-c8ccccc8)nc(-c8ccccc8)n7)c6)cc5-c5ccccc5)c4c32)c1[2H] ; [2H]c1cc([2H])c2c3c([2H])c4c(oc5c([2H])c([2H])c([2H])c([2H])c54)c([2H])c3n(-c3ccc(-c4nc(-c5ccccc5)nc(-c5ccccc5)n4)cc3-c3ccc4oc5ccccc5c4c3)c2c1[2H]]

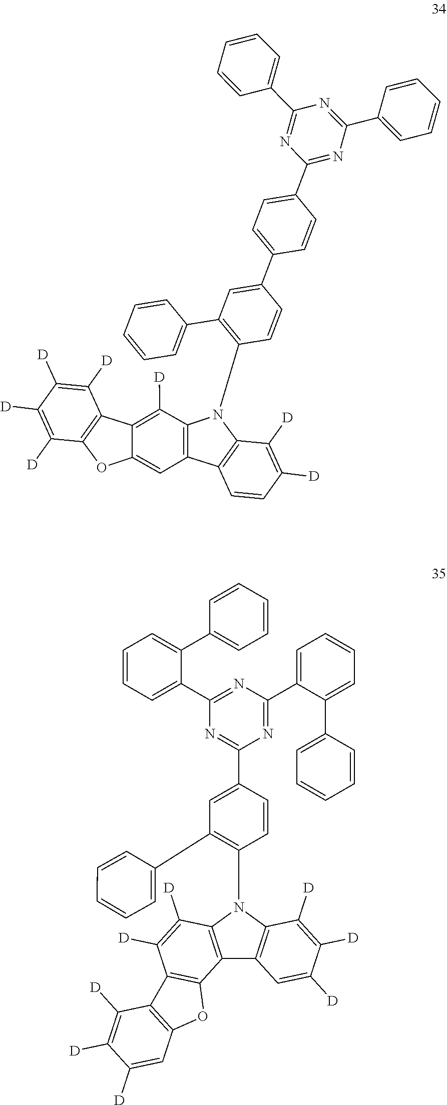 [SMILES: [2H]c1ccc2c3cc4oc5c([2H])c([2H])c([2H])c([2H])c5c4c([2H])c3n(-c3ccc(-c4ccc(-c5nc(-c6ccccc6)nc(-c6ccccc6)n5)cc4)cc3-c3ccccc3)c2c1[2H] ; [2H]c1cc2oc3c(c([2H])c([2H])c4c3c3cc([2H])c([2H])c([2H])c3n4-c3ccc(-c4nc(-c5ccccc5-c5ccccc5)nc(-c5ccccc5-c5ccccc5)n4)cc3-c3ccccc3)c2c([2H])c1[2H]]

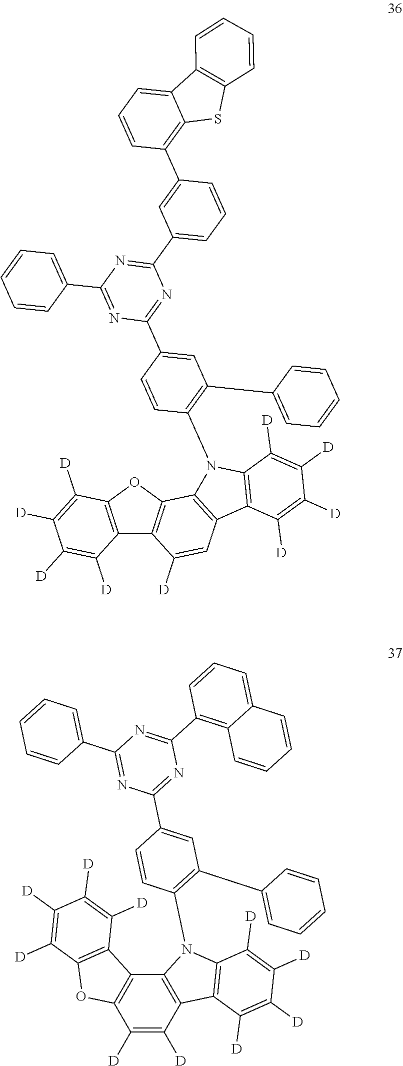 [SMILES: [2H]c1c([2H])c([2H])c2c(oc3c2c([2H])cc2c4c([2H])c([2H])c([2H])c([2H])c4n(-c4ccc(-c5nc(-c6ccccc6)nc(-c6cccc(-c7cccc8c7sc7ccccc78)c6)n5)cc4-c4ccccc4)c23)c1[2H] ; [2H]c1c([2H])c([2H])c2c(oc3c([2H])c([2H])c4c5c([2H])c([2H])c([2H])c([2H])c5n(-c5ccc(-c6nc(-c7ccccc7)nc(-c7cccc8ccccc78)n6)cc5-c5ccccc5)c4c32)c1[2H]]

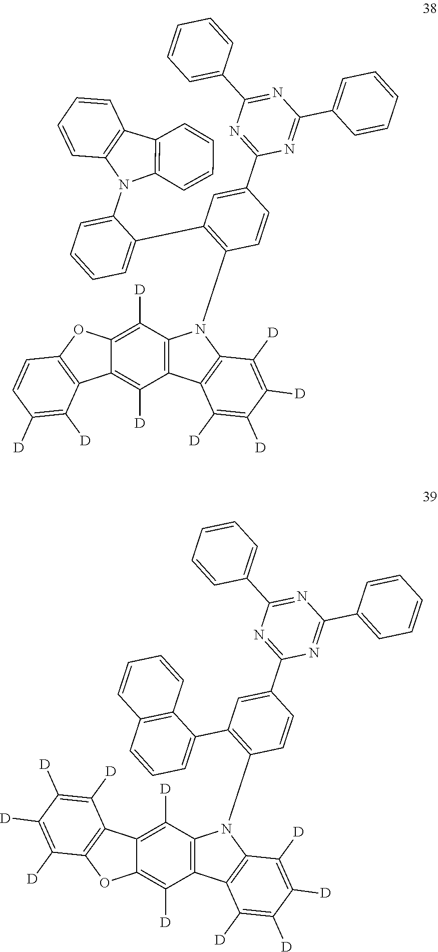 [SMILES: [2H]c1ccc2oc3c([2H])c4c(c([2H])c3c2c1[2H])c1c([2H])c([2H])c([2H])c([2H])c1n4-c1ccc(-c2nc(-c3ccccc3)nc(-c3ccccc3)n2)cc1-c1ccccc1-n1c2ccccc2c2ccccc21 ; [2H]c1c([2H])c([2H])c2c(oc3c([2H])c4c5c([2H])c([2H])c([2H])c([2H])c5n(-c5ccc(-c6nc(-c7ccccc7)nc(-c7ccccc7)n6)cc5-c5cccc6ccccc56)c4c([2H])c32)c1[2H]]

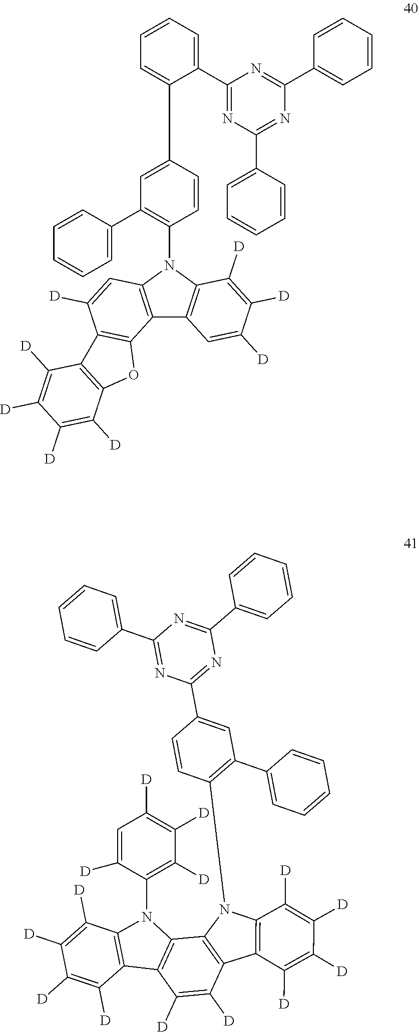 [SMILES: [2H]c1cc2c3c4oc5c([2H])c([2H])c([2H])c([2H])c5c4c([2H])cc3n(-c3ccc(-c4ccccc4-c4nc(-c5ccccc5)nc(-c5ccccc5)n4)cc3-c3ccccc3)c2c([2H])c1[2H] ; [2H]c1cc([2H])c(-n2c3c([2H])c([2H])c([2H])c([2H])c3c3c([2H])c([2H])c4c5c([2H])c([2H])c([2H])c([2H])c5n(-c5ccc(-c6nc(-c7ccccc7)nc(-c7ccccc7)n6)cc5-c5ccccc5)c4c32)c([2H])c1[2H]]

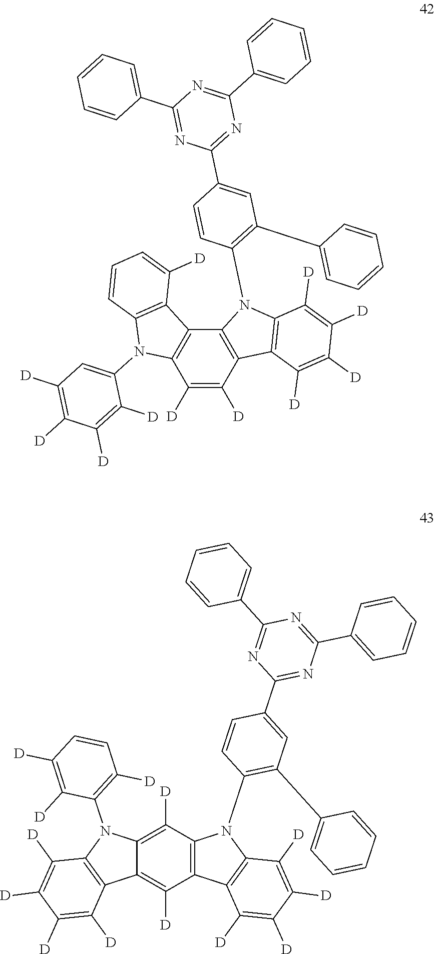 [SMILES: [2H]c1cc(-n2c3cccc([2H])c3c3c2c([2H])c([2H])c2c4c([2H])c([2H])c([2H])c([2H])c4n(-c4ccc(-c5nc(-c6ccccc6)nc(-c6ccccc6)n5)cc4-c4ccccc4)c23)c([2H])c([2H])c1[2H] ; [2H]c1ccc([2H])c(-n2c3c([2H])c([2H])c([2H])c([2H])c3c3c([2H])c4c5c([2H])c([2H])c([2H])c([2H])c5n(-c5ccc(-c6nc(-c7ccccc7)nc(-c7ccccc7)n6)cc5-c5ccccc5)c4c([2H])c32)c1[2H]]

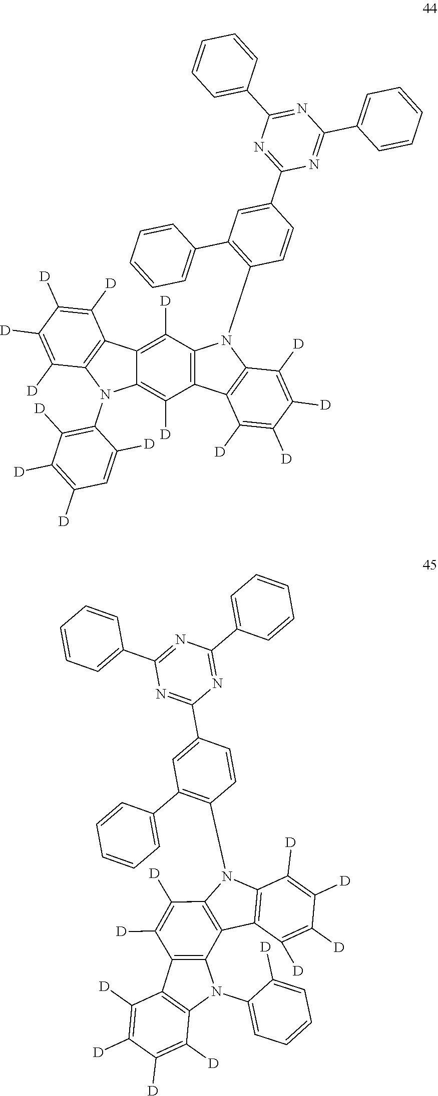 [SMILES: [2H]c1cc([2H])c(-n2c3c([2H])c([2H])c([2H])c([2H])c3c3c([2H])c4c(c([2H])c32)c2c([2H])c([2H])c([2H])c([2H])c2n4-c2ccc(-c3nc(-c4ccccc4)nc(-c4ccccc4)n3)cc2-c2ccccc2)c([2H])c1[2H] ; [2H]c1ccccc1-n1c2c([2H])c([2H])c([2H])c([2H])c2c2c([2H])c([2H])c3c(c4c([2H])c([2H])c([2H])c([2H])c4n3-c3ccc(-c4nc(-c5ccccc5)nc(-c5ccccc5)n4)cc3-c3ccccc3)c21]

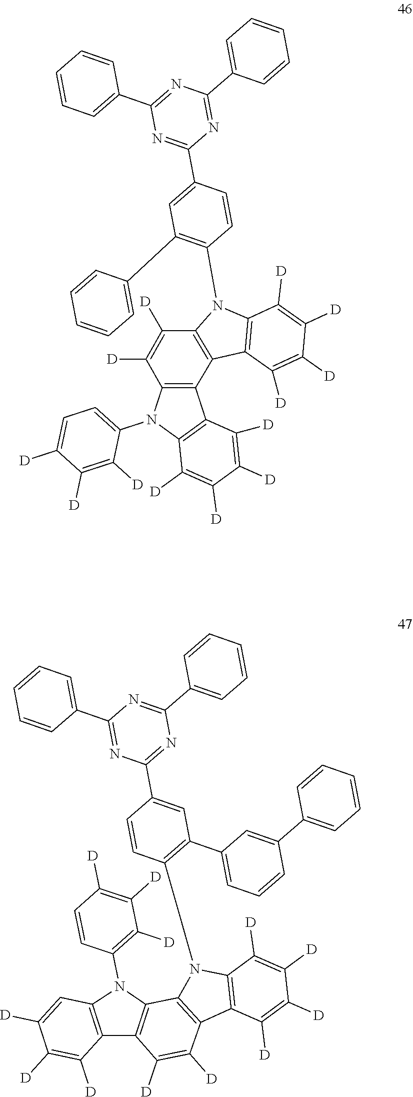 [SMILES: [2H]c1ccc(-n2c3c([2H])c([2H])c([2H])c([2H])c3c3c4c5c([2H])c([2H])c([2H])c([2H])c5n(-c5ccc(-c6nc(-c7ccccc7)nc(-c7ccccc7)n6)cc5-c5ccccc5)c4c([2H])c([2H])c32)c([2H])c1[2H] ; [2H]c1ccc(-n2c3cc([2H])c([2H])c([2H])c3c3c([2H])c([2H])c4c5c([2H])c([2H])c([2H])c([2H])c5n(-c5ccc(-c6nc(-c7ccccc7)nc(-c7ccccc7)n6)cc5-c5cccc(-c6ccccc6)c5)c4c32)c([2H])c1[2H]]

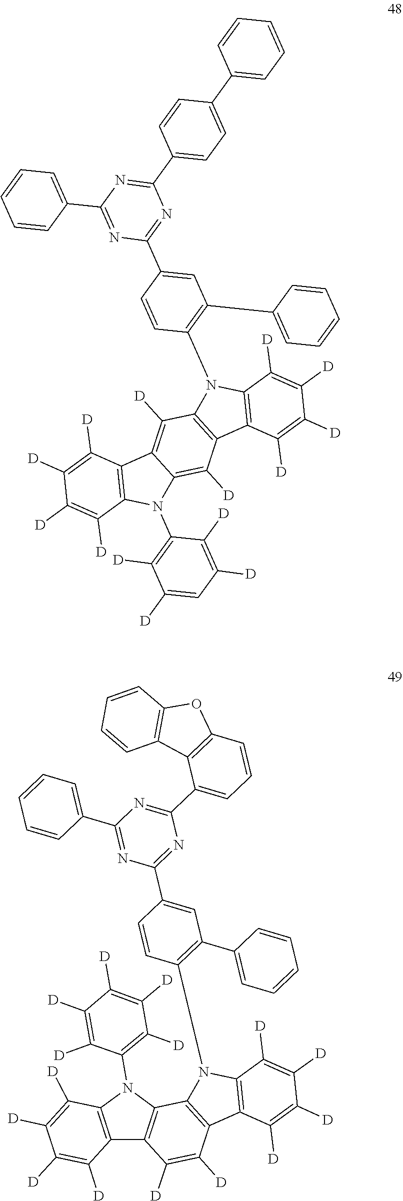 [SMILES: [2H]c1cc([2H])c([2H])c(-n2c3c([2H])c([2H])c([2H])c([2H])c3c3c([2H])c4c(c([2H])c32)c2c([2H])c([2H])c([2H])c([2H])c2n4-c2ccc(-c3nc(-c4ccccc4)nc(-c4ccc(-c5ccccc5)cc4)n3)cc2-c2ccccc2)c1[2H] ; [2H]c1c([2H])c([2H])c(-n2c3c([2H])c([2H])c([2H])c([2H])c3c3c([2H])c([2H])c4c5c([2H])c([2H])c([2H])c([2H])c5n(-c5ccc(-c6nc(-c7ccccc7)nc(-c7cccc8oc9ccccc9c78)n6)cc5-c5ccccc5)c4c32)c([2H])c1[2H]]

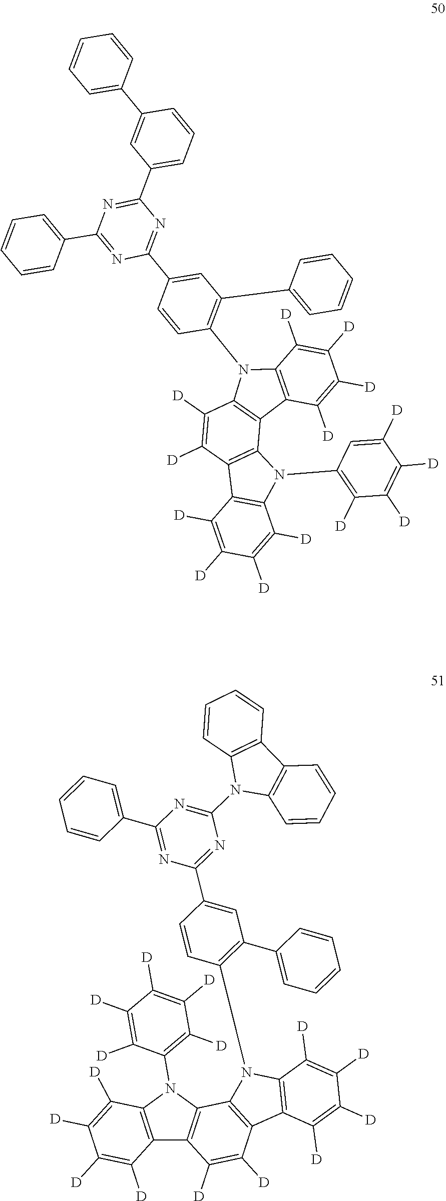 [SMILES: [2H]c1cc(-n2c3c([2H])c([2H])c([2H])c([2H])c3c3c([2H])c([2H])c4c(c5c([2H])c([2H])c([2H])c([2H])c5n4-c4ccc(-c5nc(-c6ccccc6)nc(-c6cccc(-c7ccccc7)c6)n5)cc4-c4ccccc4)c32)c([2H])c([2H])c1[2H] ; [2H]c1c([2H])c([2H])c(-n2c3c([2H])c([2H])c([2H])c([2H])c3c3c([2H])c([2H])c4c5c([2H])c([2H])c([2H])c([2H])c5n(-c5ccc(-c6nc(-c7ccccc7)nc(-n7c8ccccc8c8ccccc87)n6)cc5-c5ccccc5)c4c32)c([2H])c1[2H]]

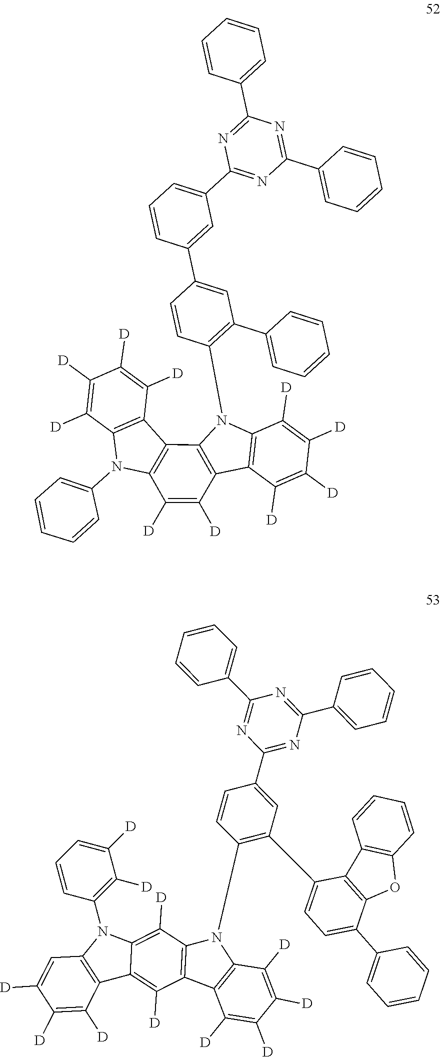 [SMILES: [2H]c1c([2H])c([2H])c2c(c1[2H])c1c(c([2H])c([2H])c3c4c([2H])c([2H])c([2H])c([2H])c4n(-c4ccc(-c5cccc(-c6nc(-c7ccccc7)nc(-c7ccccc7)n6)c5)cc4-c4ccccc4)c31)n2-c1ccccc1 ; [2H]c1cccc(-n2c3cc([2H])c([2H])c([2H])c3c3c([2H])c4c5c([2H])c([2H])c([2H])c([2H])c5n(-c5ccc(-c6nc(-c7ccccc7)nc(-c7ccccc7)n6)cc5-c5ccc(-c6ccccc6)c6oc7ccccc7c56)c4c([2H])c32)c1[2H]]

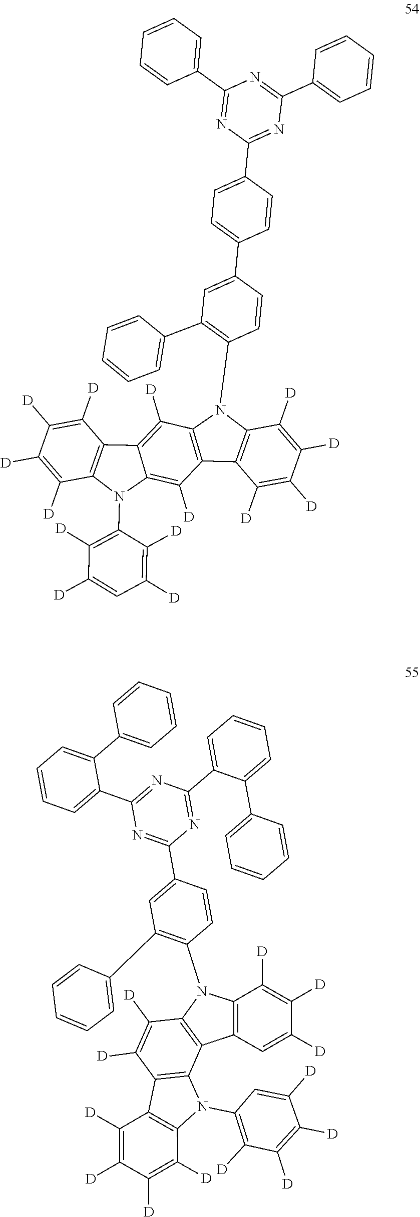 [SMILES: [2H]c1cc([2H])c([2H])c(-n2c3c([2H])c([2H])c([2H])c([2H])c3c3c([2H])c4c(c([2H])c32)c2c([2H])c([2H])c([2H])c([2H])c2n4-c2ccc(-c3ccc(-c4nc(-c5ccccc5)nc(-c5ccccc5)n4)cc3)cc2-c2ccccc2)c1[2H] ; [2H]c1cc(-n2c3c([2H])c([2H])c([2H])c([2H])c3c3c([2H])c([2H])c4c(c5cc([2H])c([2H])c([2H])c5n4-c4ccc(-c5nc(-c6ccccc6-c6ccccc6)nc(-c6ccccc6-c6ccccc6)n5)cc4-c4ccccc4)c32)c([2H])c([2H])c1[2H]]

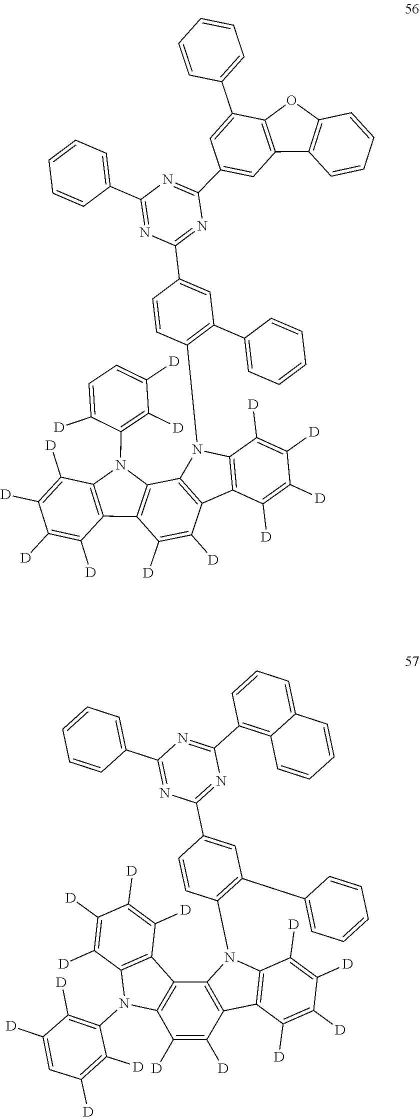 [SMILES: [2H]c1ccc([2H])c(-n2c3c([2H])c([2H])c([2H])c([2H])c3c3c([2H])c([2H])c4c5c([2H])c([2H])c([2H])c([2H])c5n(-c5ccc(-c6nc(-c7ccccc7)nc(-c7cc(-c8ccccc8)c8oc9ccccc9c8c7)n6)cc5-c5ccccc5)c4c32)c1[2H] ; [2H]c1cc([2H])c([2H])c(-n2c3c([2H])c([2H])c([2H])c([2H])c3c3c2c([2H])c([2H])c2c4c([2H])c([2H])c([2H])c([2H])c4n(-c4ccc(-c5nc(-c6ccccc6)nc(-c6cccc7ccccc67)n5)cc4-c4ccccc4)c23)c1[2H]]

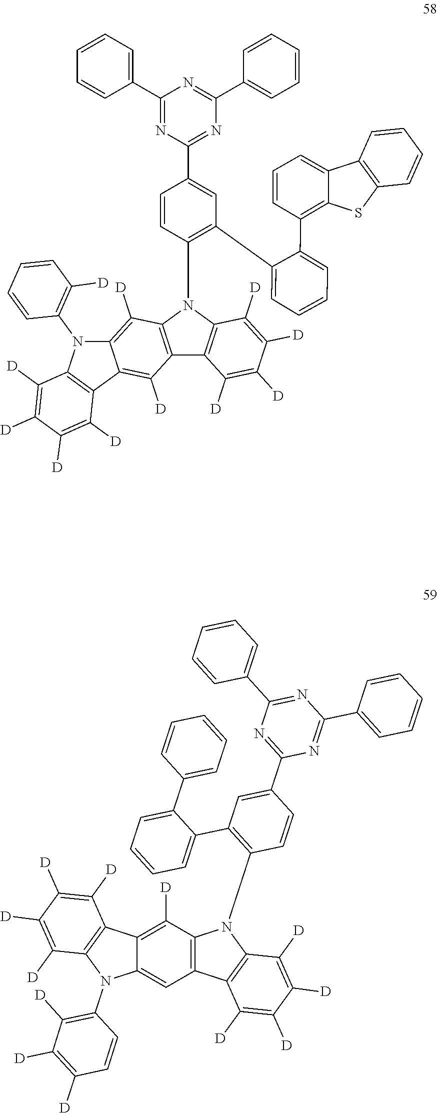 [SMILES: [2H]c1ccccc1-n1c2c([2H])c([2H])c([2H])c([2H])c2c2c([2H])c3c4c([2H])c([2H])c([2H])c([2H])c4n(-c4ccc(-c5nc(-c6ccccc6)nc(-c6ccccc6)n5)cc4-c4ccccc4-c4cccc5c4sc4ccccc45)c3c([2H])c21 ; [2H]c1ccc(-n2c3cc4c5c([2H])c([2H])c([2H])c([2H])c5n(-c5ccc(-c6nc(-c7ccccc7)nc(-c7ccccc7)n6)cc5-c5ccccc5-c5ccccc5)c4c([2H])c3c3c([2H])c([2H])c([2H])c([2H])c32)c([2H])c1[2H]]

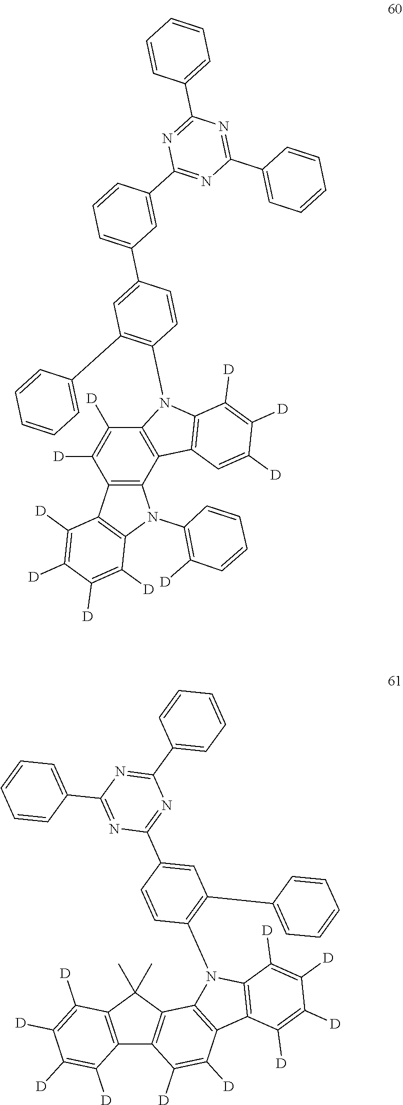 [SMILES: [2H]c1ccccc1-n1c2c([2H])c([2H])c([2H])c([2H])c2c2c([2H])c([2H])c3c(c4cc([2H])c([2H])c([2H])c4n3-c3ccc(-c4cccc(-c5nc(-c6ccccc6)nc(-c6ccccc6)n5)c4)cc3-c3ccccc3)c21 ; [2H]c1c([2H])c([2H])c2c(c1[2H])-c1c([2H])c([2H])c3c4c([2H])c([2H])c([2H])c([2H])c4n(-c4ccc(-c5nc(-c6ccccc6)nc(-c6ccccc6)n5)cc4-c4ccccc4)c3c1C2(C)C]

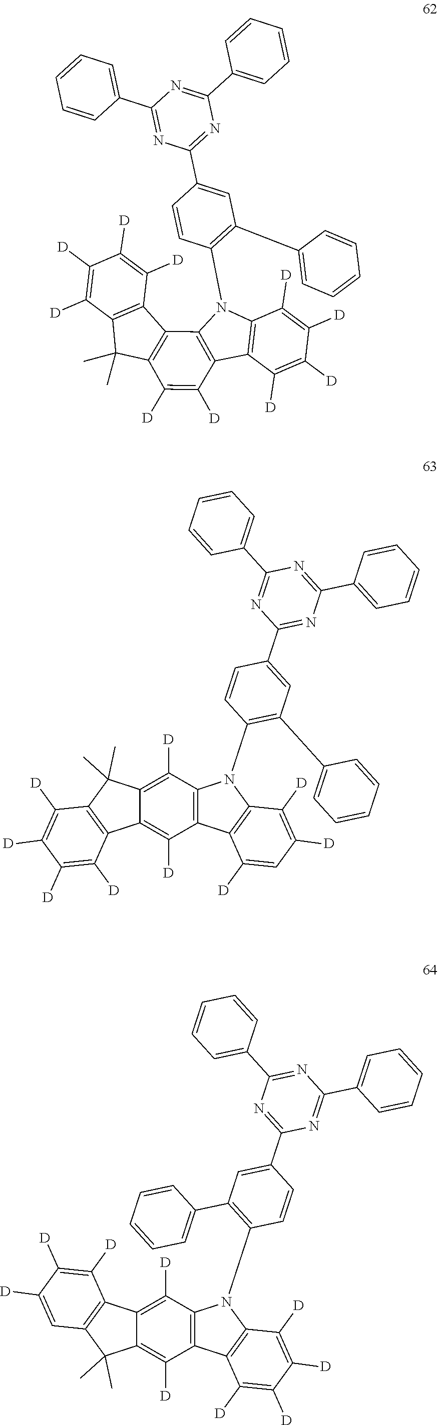 [SMILES: [2H]c1c([2H])c([2H])c2c(c1[2H])-c1c(c([2H])c([2H])c3c4c([2H])c([2H])c([2H])c([2H])c4n(-c4ccc(-c5nc(-c6ccccc6)nc(-c6ccccc6)n5)cc4-c4ccccc4)c13)C2(C)C ; [2H]c1cc([2H])c2c3c([2H])c4c(c([2H])c3n(-c3ccc(-c5nc(-c6ccccc6)nc(-c6ccccc6)n5)cc3-c3ccccc3)c2c1[2H])C(C)(C)c1c([2H])c([2H])c([2H])c([2H])c1-4 ; [2H]c1cc2c(c([2H])c1[2H])-c1c(c([2H])c3c4c([2H])c([2H])c([2H])c([2H])c4n(-c4ccc(-c5nc(-c6ccccc6)nc(-c6ccccc6)n5)cc4-c4ccccc4)c3c1[2H])C2(C)C]

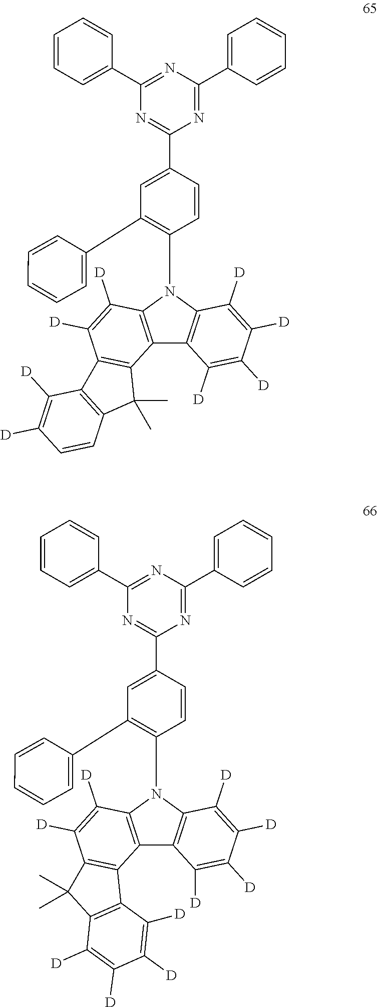 [SMILES: [2H]c1ccc2c(c1[2H])-c1c([2H])c([2H])c3c(c1C2(C)C)c1c([2H])c([2H])c([2H])c([2H])c1n3-c1ccc(-c2nc(-c3ccccc3)nc(-c3ccccc3)n2)cc1-c1ccccc1 ; [2H]c1c([2H])c([2H])c2c(c1[2H])-c1c(c([2H])c([2H])c3c1c1c([2H])c([2H])c([2H])c([2H])c1n3-c1ccc(-c3nc(-c4ccccc4)nc(-c4ccccc4)n3)cc1-c1ccccc1)C2(C)C]

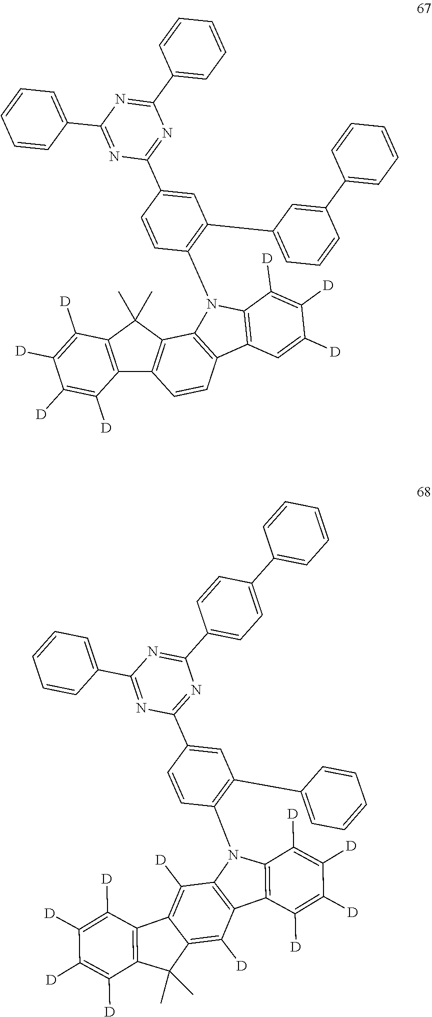 [SMILES: [2H]c1cc2c3ccc4c(c3n(-c3ccc(-c5nc(-c6ccccc6)nc(-c6ccccc6)n5)cc3-c3cccc(-c5ccccc5)c3)c2c([2H])c1[2H])C(C)(C)c1c([2H])c([2H])c([2H])c([2H])c1-4 ; [2H]c1c([2H])c([2H])c2c(c1[2H])-c1c(c([2H])c3c4c([2H])c([2H])c([2H])c([2H])c4n(-c4ccc(-c5nc(-c6ccccc6)nc(-c6ccc(-c7ccccc7)cc6)n5)cc4-c4ccccc4)c3c1[2H])C2(C)C]

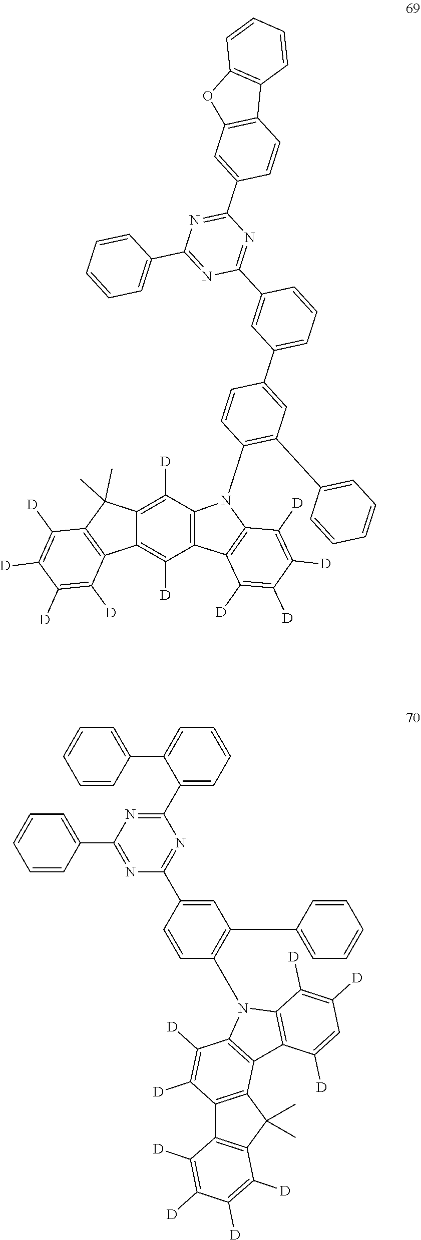 [SMILES: [2H]c1c([2H])c([2H])c2c(c1[2H])-c1c(c([2H])c3c(c1[2H])c1c([2H])c([2H])c([2H])c([2H])c1n3-c1ccc(-c3cccc(-c4nc(-c5ccccc5)nc(-c5ccc6c(c5)oc5ccccc56)n4)c3)cc1-c1ccccc1)C2(C)C ; [2H]c1cc([2H])c2c3c4c(c([2H])c([2H])c3n(-c3ccc(-c5nc(-c6ccccc6)nc(-c6ccccc6-c6ccccc6)n5)cc3-c3ccccc3)c2c1[2H])-c1c([2H])c([2H])c([2H])c([2H])c1C4(C)C]

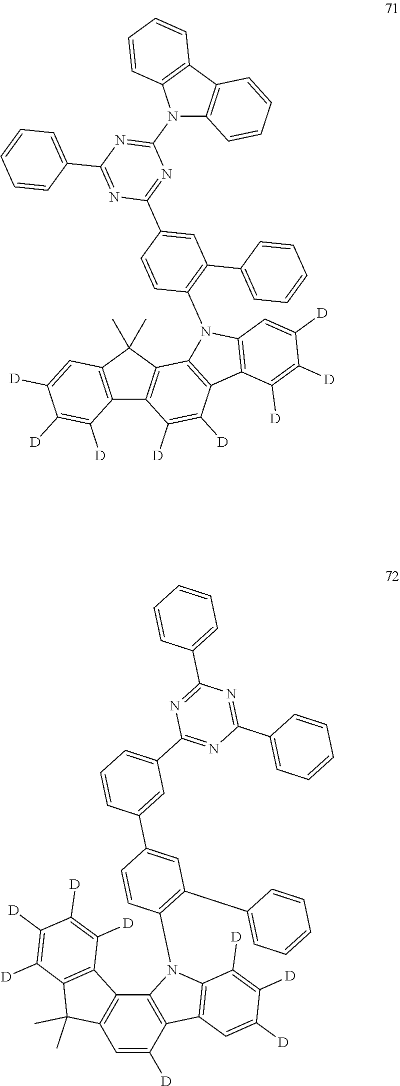 [SMILES: [2H]c1cc2c(c([2H])c1[2H])-c1c([2H])c([2H])c3c4c([2H])c([2H])c([2H])cc4n(-c4ccc(-c5nc(-c6ccccc6)nc(-n6c7ccccc7c7ccccc76)n5)cc4-c4ccccc4)c3c1C2(C)C ; [2H]c1cc2c3c([2H])cc4c(c3n(-c3ccc(-c5cccc(-c6nc(-c7ccccc7)nc(-c7ccccc7)n6)c5)cc3-c3ccccc3)c2c([2H])c1[2H])-c1c([2H])c([2H])c([2H])c([2H])c1C4(C)C]

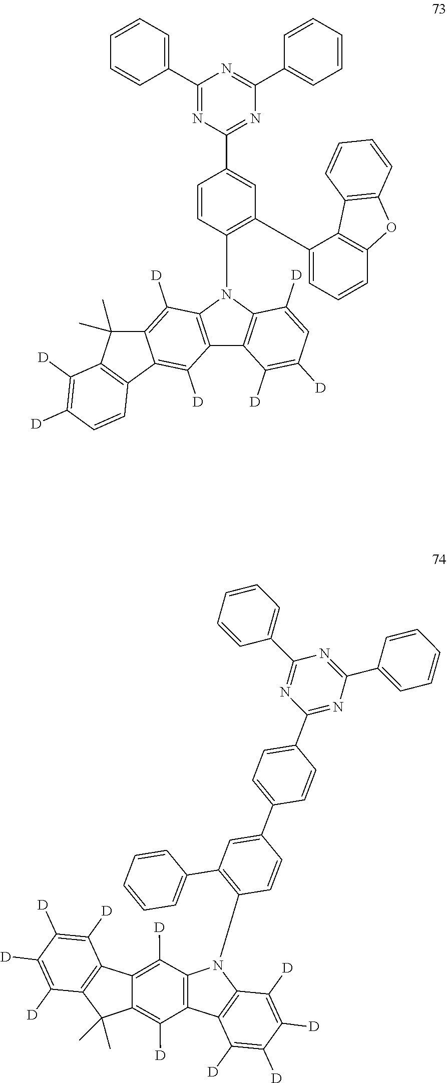 [SMILES: [2H]c1ccc2c(c1[2H])C(C)(C)c1c-2c([2H])c2c3c([2H])c([2H])cc([2H])c3n(-c3ccc(-c4nc(-c5ccccc5)nc(-c5ccccc5)n4)cc3-c3cccc4oc5ccccc5c34)c2c1[2H] ; [2H]c1c([2H])c([2H])c2c(c1[2H])-c1c(c([2H])c3c4c([2H])c([2H])c([2H])c([2H])c4n(-c4ccc(-c5ccc(-c6nc(-c7ccccc7)nc(-c7ccccc7)n6)cc5)cc4-c4ccccc4)c3c1[2H])C2(C)C]

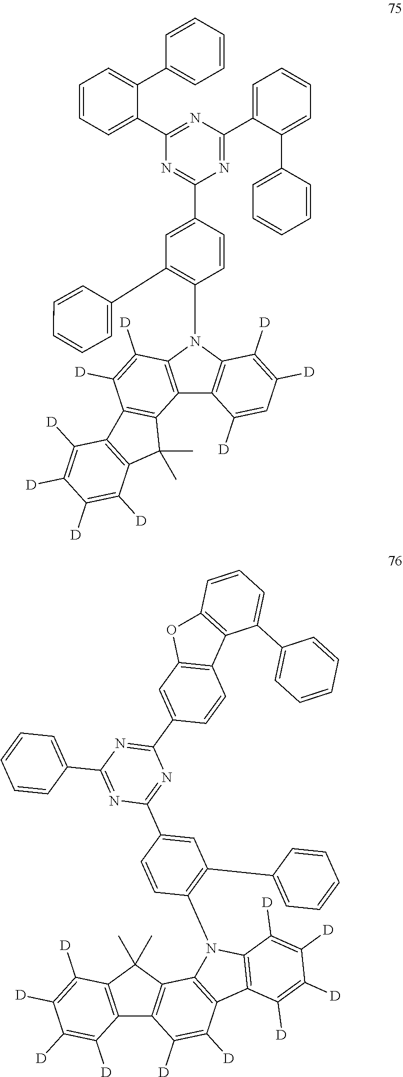 [SMILES: [2H]c1cc([2H])c2c3c4c(c([2H])c([2H])c3n(-c3ccc(-c5nc(-c6ccccc6-c6ccccc6)nc(-c6ccccc6-c6ccccc6)n5)cc3-c3ccccc3)c2c1[2H])-c1c([2H])c([2H])c([2H])c([2H])c1C4(C)C ; [2H]c1c([2H])c([2H])c2c(c1[2H])-c1c([2H])c([2H])c3c4c([2H])c([2H])c([2H])c([2H])c4n(-c4ccc(-c5nc(-c6ccccc6)nc(-c6ccc7c(c6)oc6cccc(-c8ccccc8)c67)n5)cc4-c4ccccc4)c3c1C2(C)C]

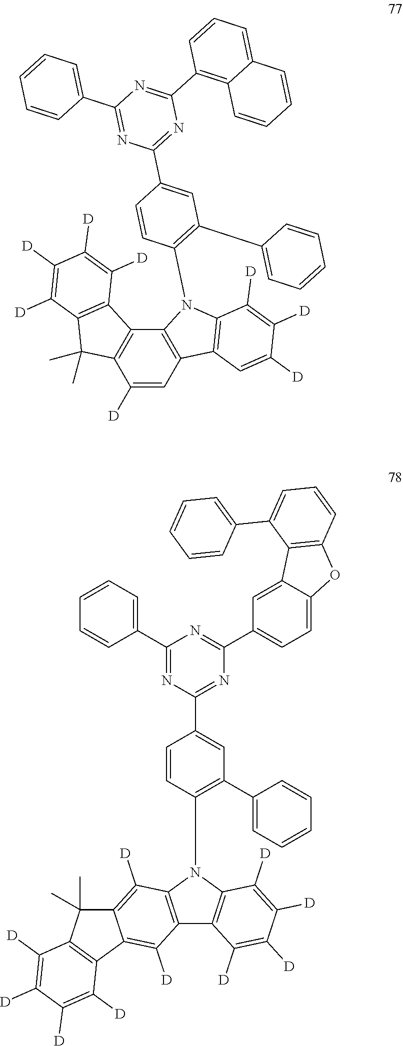 [SMILES: [2H]c1cc2c3cc([2H])c4c(c3n(-c3ccc(-c5nc(-c6ccccc6)nc(-c6cccc7ccccc67)n5)cc3-c3ccccc3)c2c([2H])c1[2H])-c1c([2H])c([2H])c([2H])c([2H])c1C4(C)C ; [2H]c1c([2H])c([2H])c2c(c1[2H])-c1c(c([2H])c3c(c1[2H])c1c([2H])c([2H])c([2H])c([2H])c1n3-c1ccc(-c3nc(-c4ccccc4)nc(-c4ccc5oc6cccc(-c7ccccc7)c6c5c4)n3)cc1-c1ccccc1)C2(C)C]

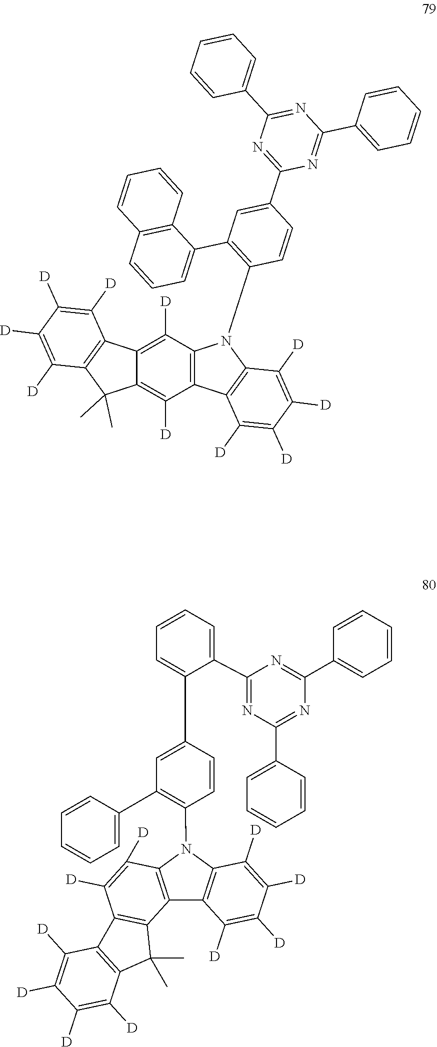 [SMILES: [2H]c1c([2H])c([2H])c2c(c1[2H])-c1c(c([2H])c3c4c([2H])c([2H])c([2H])c([2H])c4n(-c4ccc(-c5nc(-c6ccccc6)nc(-c6ccccc6)n5)cc4-c4cccc5ccccc45)c3c1[2H])C2(C)C ; [2H]c1c([2H])c([2H])c2c(c1[2H])-c1c([2H])c([2H])c3c(c1C2(C)C)c1c([2H])c([2H])c([2H])c([2H])c1n3-c1ccc(-c2ccccc2-c2nc(-c3ccccc3)nc(-c3ccccc3)n2)cc1-c1ccccc1]

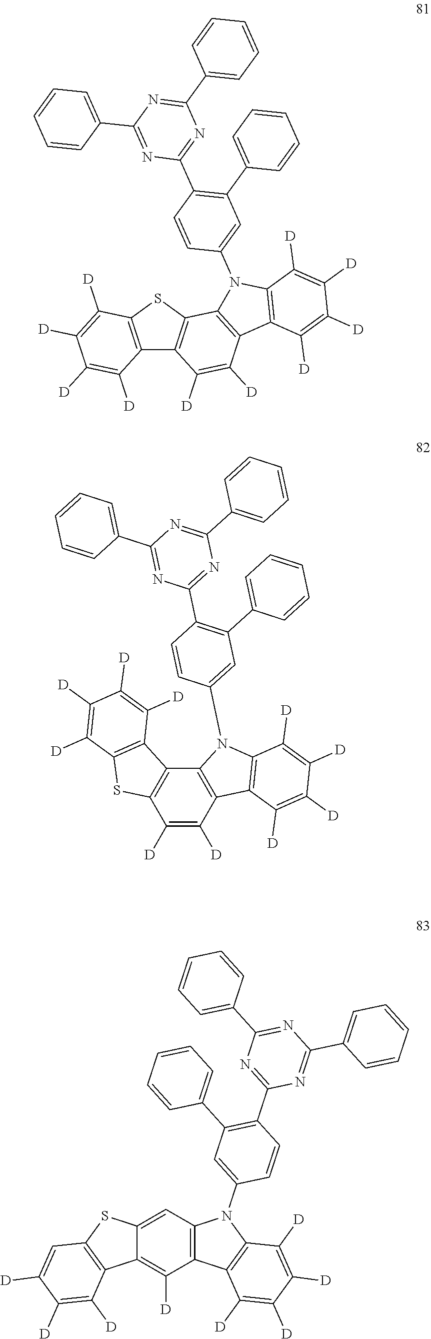 [SMILES: [2H]c1c([2H])c([2H])c2c(sc3c2c([2H])c([2H])c2c4c([2H])c([2H])c([2H])c([2H])c4n(-c4ccc(-c5nc(-c6ccccc6)nc(-c6ccccc6)n5)c(-c5ccccc5)c4)c32)c1[2H] ; [2H]c1c([2H])c([2H])c2c(sc3c([2H])c([2H])c4c5c([2H])c([2H])c([2H])c([2H])c5n(-c5ccc(-c6nc(-c7ccccc7)nc(-c7ccccc7)n6)c(-c6ccccc6)c5)c4c32)c1[2H] ; [2H]c1cc2sc3cc4c(c([2H])c3c2c([2H])c1[2H])c1c([2H])c([2H])c([2H])c([2H])c1n4-c1ccc(-c2nc(-c3ccccc3)nc(-c3ccccc3)n2)c(-c2ccccc2)c1]

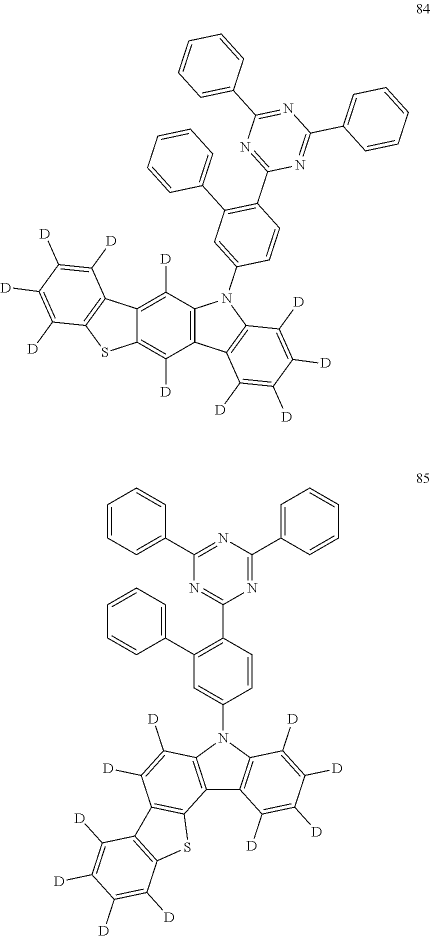 [SMILES: [2H]c1c([2H])c([2H])c2c(sc3c([2H])c4c5c([2H])c([2H])c([2H])c([2H])c5n(-c5ccc(-c6nc(-c7ccccc7)nc(-c7ccccc7)n6)c(-c6ccccc6)c5)c4c([2H])c32)c1[2H] ; [2H]c1c([2H])c([2H])c2c(sc3c2c([2H])c([2H])c2c3c3c([2H])c([2H])c([2H])c([2H])c3n2-c2ccc(-c3nc(-c4ccccc4)nc(-c4ccccc4)n3)c(-c3ccccc3)c2)c1[2H]]

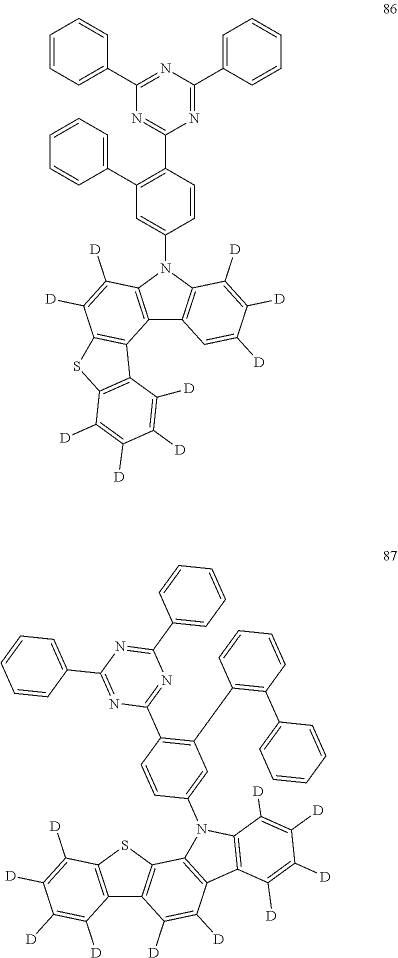 [SMILES: [2H]c1cc2c3c4c(sc5c([2H])c([2H])c([2H])c([2H])c54)c([2H])c([2H])c3n(-c3ccc(-c4nc(-c5ccccc5)nc(-c5ccccc5)n4)c(-c4ccccc4)c3)c2c([2H])c1[2H] ; [2H]c1c([2H])c([2H])c2c(sc3c2c([2H])c([2H])c2c4c([2H])c([2H])c([2H])c([2H])c4n(-c4ccc(-c5nc(-c6ccccc6)nc(-c6ccccc6)n5)c(-c5ccccc5-c5ccccc5)c4)c32)c1[2H]]

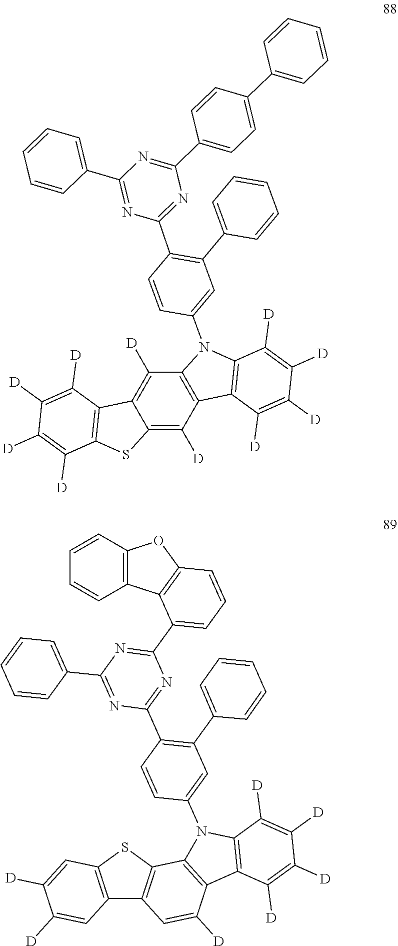 [SMILES: [2H]c1c([2H])c([2H])c2c(sc3c([2H])c4c5c([2H])c([2H])c([2H])c([2H])c5n(-c5ccc(-c6nc(-c7ccccc7)nc(-c7ccc(-c8ccccc8)cc7)n6)c(-c6ccccc6)c5)c4c([2H])c32)c1[2H] ; [2H]c1cc2sc3c(cc([2H])c4c5c([2H])c([2H])c([2H])c([2H])c5n(-c5ccc(-c6nc(-c7ccccc7)nc(-c7cccc8oc9ccccc9c78)n6)c(-c6ccccc6)c5)c34)c2cc1[2H]]

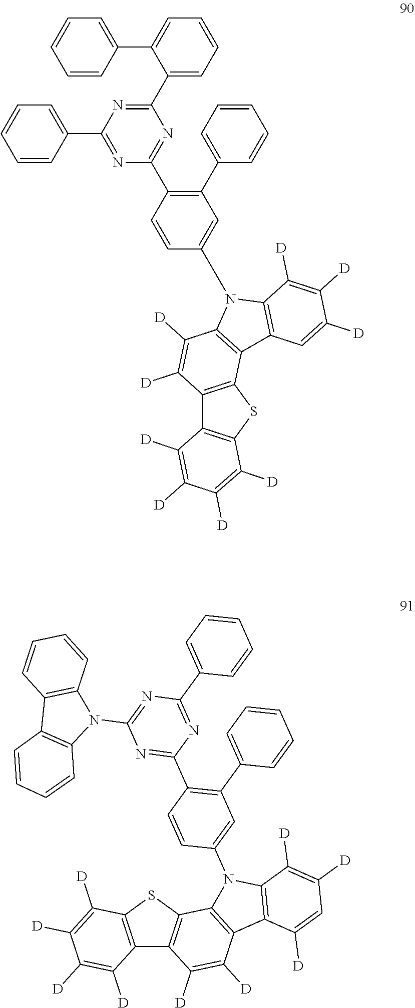 [SMILES: [2H]c1cc2c3c4sc5c([2H])c([2H])c([2H])c([2H])c5c4c([2H])c([2H])c3n(-c3ccc(-c4nc(-c5ccccc5)nc(-c5ccccc5-c5ccccc5)n4)c(-c4ccccc4)c3)c2c([2H])c1[2H] ; [2H]c1cc([2H])c2c3c([2H])c([2H])c4c(sc5c([2H])c([2H])c([2H])c([2H])c54)c3n(-c3ccc(-c4nc(-c5ccccc5)nc(-n5c6ccccc6c6ccccc65)n4)c(-c4ccccc4)c3)c2c1[2H]]

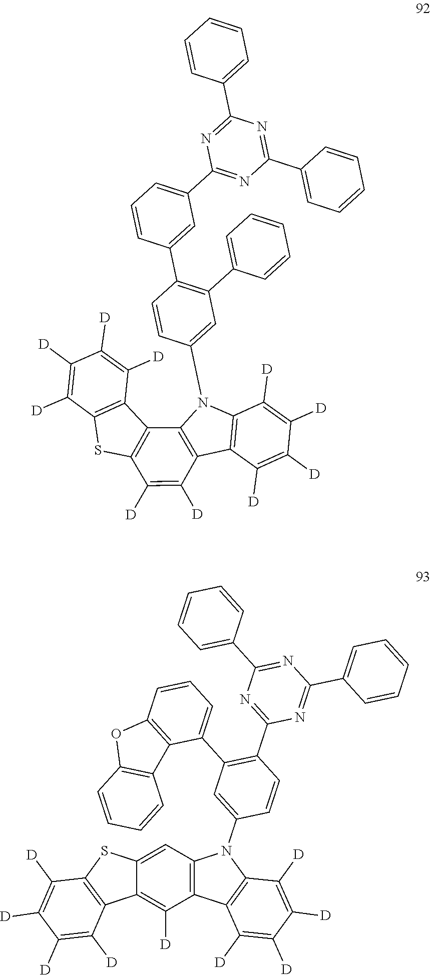 [SMILES: [2H]c1c([2H])c([2H])c2c(sc3c([2H])c([2H])c4c5c([2H])c([2H])c([2H])c([2H])c5n(-c5ccc(-c6cccc(-c7nc(-c8ccccc8)nc(-c8ccccc8)n7)c6)c(-c6ccccc6)c5)c4c32)c1[2H] ; [2H]c1c([2H])c([2H])c2c(sc3cc4c(c([2H])c32)c2c([2H])c([2H])c([2H])c([2H])c2n4-c2ccc(-c3nc(-c4ccccc4)nc(-c4ccccc4)n3)c(-c3cccc4oc5ccccc5c34)c2)c1[2H]]

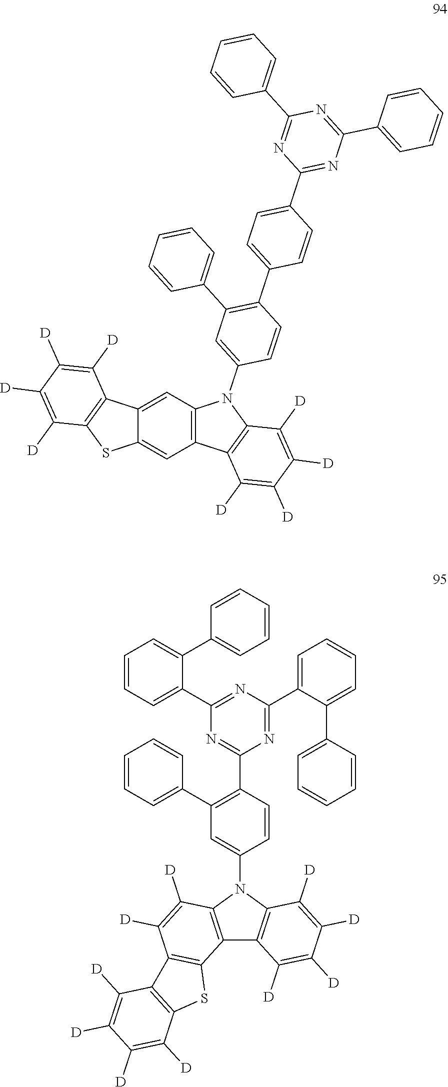 [SMILES: [2H]c1c([2H])c([2H])c2c(sc3cc4c5c([2H])c([2H])c([2H])c([2H])c5n(-c5ccc(-c6ccc(-c7nc(-c8ccccc8)nc(-c8ccccc8)n7)cc6)c(-c6ccccc6)c5)c4cc32)c1[2H] ; [2H]c1c([2H])c([2H])c2c(sc3c2c([2H])c([2H])c2c3c3c([2H])c([2H])c([2H])c([2H])c3n2-c2ccc(-c3nc(-c4ccccc4-c4ccccc4)nc(-c4ccccc4-c4ccccc4)n3)c(-c3ccccc3)c2)c1[2H]]

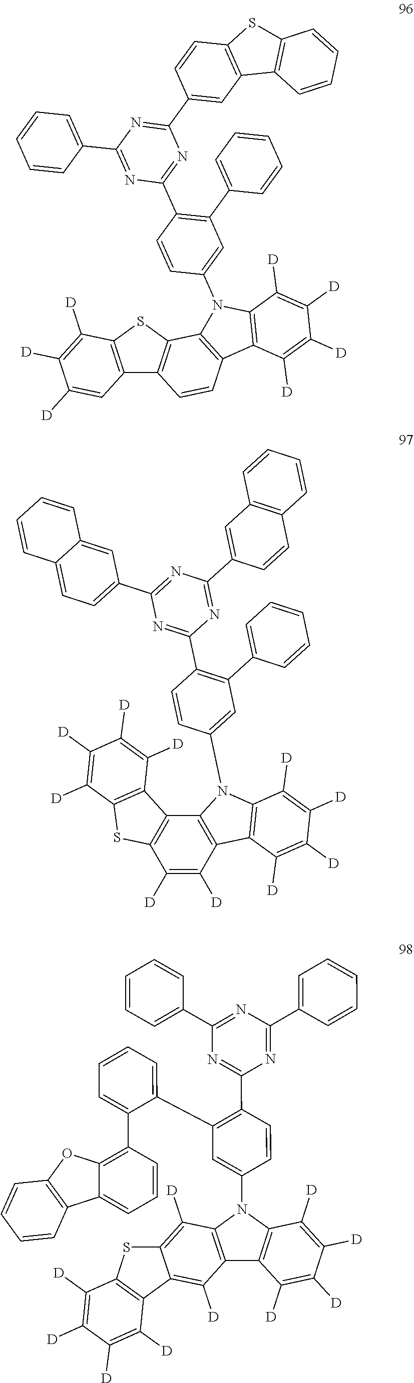 [SMILES: [2H]c1cc2c(sc3c2ccc2c4c([2H])c([2H])c([2H])c([2H])c4n(-c4ccc(-c5nc(-c6ccccc6)nc(-c6ccc7sc8ccccc8c7c6)n5)c(-c5ccccc5)c4)c23)c([2H])c1[2H] ; [2H]c1c([2H])c([2H])c2c(sc3c([2H])c([2H])c4c5c([2H])c([2H])c([2H])c([2H])c5n(-c5ccc(-c6nc(-c7ccc8ccccc8c7)nc(-c7ccc8ccccc8c7)n6)c(-c6ccccc6)c5)c4c32)c1[2H] ; [2H]c1c([2H])c([2H])c2c(sc3c([2H])c4c(c([2H])c32)c2c([2H])c([2H])c([2H])c([2H])c2n4-c2ccc(-c3nc(-c4ccccc4)nc(-c4ccccc4)n3)c(-c3ccccc3-c3cccc4c3oc3ccccc34)c2)c1[2H]]

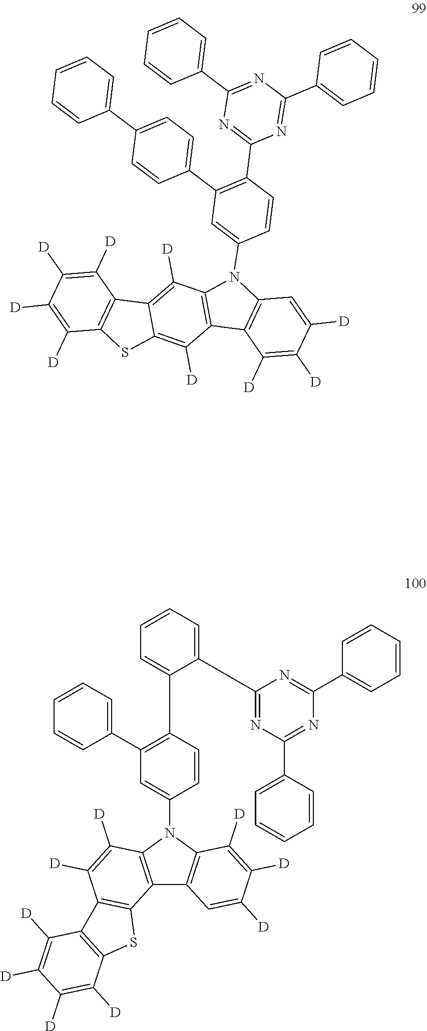 [SMILES: [2H]c1cc2c(c([2H])c1[2H])c1c([2H])c3sc4c([2H])c([2H])c([2H])c([2H])c4c3c([2H])c1n2-c1ccc(-c2nc(-c3ccccc3)nc(-c3ccccc3)n2)c(-c2ccc(-c3ccccc3)cc2)c1 ; [2H]c1cc2c3c4sc5c([2H])c([2H])c([2H])c([2H])c5c4c([2H])c([2H])c3n(-c3ccc(-c4ccccc4-c4nc(-c5ccccc5)nc(-c5ccccc5)n4)c(-c4ccccc4)c3)c2c([2H])c1[2H]]

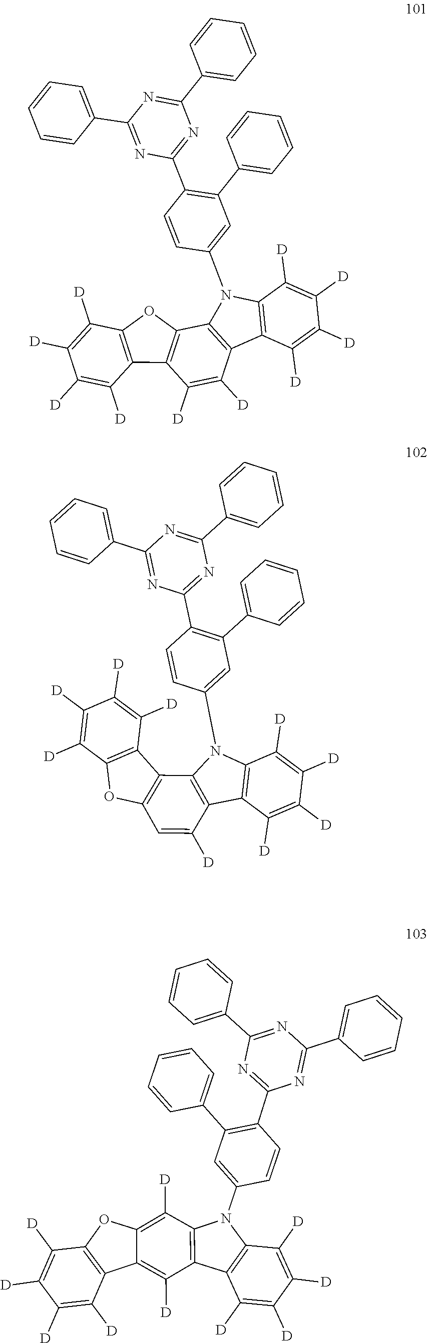 [SMILES: [2H]c1c([2H])c([2H])c2c(oc3c2c([2H])c([2H])c2c4c([2H])c([2H])c([2H])c([2H])c4n(-c4ccc(-c5nc(-c6ccccc6)nc(-c6ccccc6)n5)c(-c5ccccc5)c4)c32)c1[2H] ; [2H]c1c([2H])c([2H])c2c(oc3cc([2H])c4c5c([2H])c([2H])c([2H])c([2H])c5n(-c5ccc(-c6nc(-c7ccccc7)nc(-c7ccccc7)n6)c(-c6ccccc6)c5)c4c32)c1[2H] ; [2H]c1c([2H])c([2H])c2c(oc3c([2H])c4c(c([2H])c32)c2c([2H])c([2H])c([2H])c([2H])c2n4-c2ccc(-c3nc(-c4ccccc4)nc(-c4ccccc4)n3)c(-c3ccccc3)c2)c1[2H]]

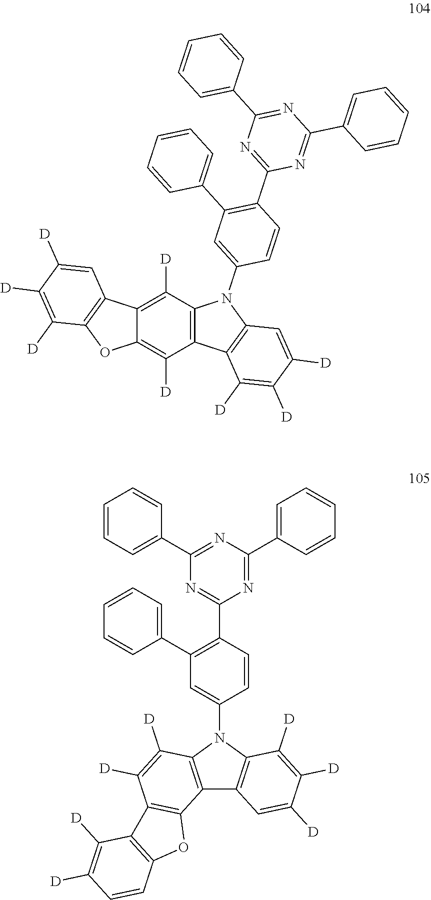 [SMILES: [2H]c1cc2c(oc3c([2H])c4c5c([2H])c([2H])c([2H])cc5n(-c5ccc(-c6nc(-c7ccccc7)nc(-c7ccccc7)n6)c(-c6ccccc6)c5)c4c([2H])c32)c([2H])c1[2H] ; [2H]c1cc2c3c4oc5ccc([2H])c([2H])c5c4c([2H])c([2H])c3n(-c3ccc(-c4nc(-c5ccccc5)nc(-c5ccccc5)n4)c(-c4ccccc4)c3)c2c([2H])c1[2H]]

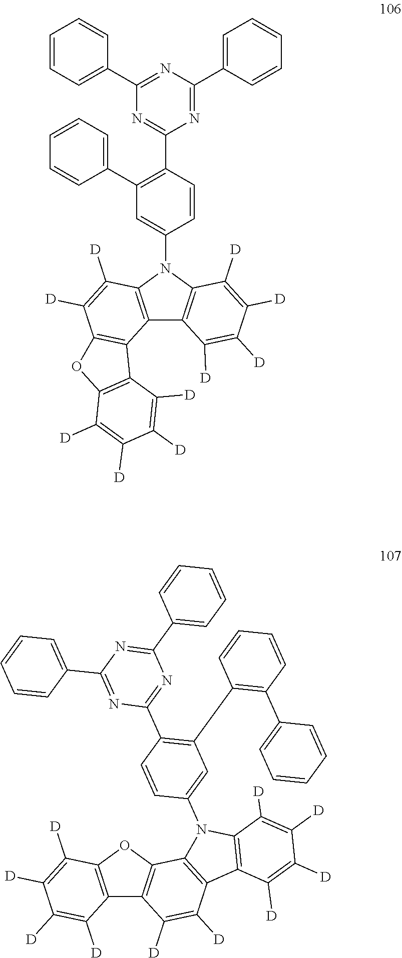 [SMILES: [2H]c1c([2H])c([2H])c2c(oc3c([2H])c([2H])c4c(c5c([2H])c([2H])c([2H])c([2H])c5n4-c4ccc(-c5nc(-c6ccccc6)nc(-c6ccccc6)n5)c(-c5ccccc5)c4)c32)c1[2H] ; [2H]c1c([2H])c([2H])c2c(oc3c2c([2H])c([2H])c2c4c([2H])c([2H])c([2H])c([2H])c4n(-c4ccc(-c5nc(-c6ccccc6)nc(-c6ccccc6)n5)c(-c5ccccc5-c5ccccc5)c4)c32)c1[2H]]

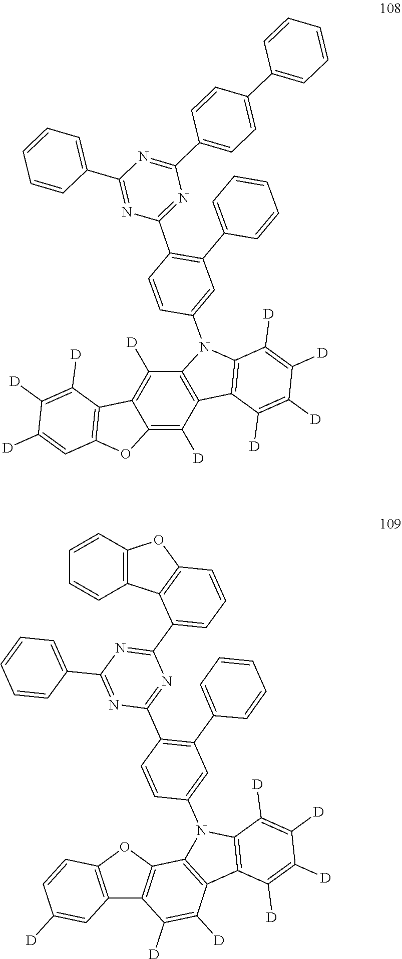 [SMILES: [2H]c1cc2oc3c([2H])c4c5c([2H])c([2H])c([2H])c([2H])c5n(-c5ccc(-c6nc(-c7ccccc7)nc(-c7ccc(-c8ccccc8)cc7)n6)c(-c6ccccc6)c5)c4c([2H])c3c2c([2H])c1[2H] ; [2H]c1ccc2oc3c(c([2H])c([2H])c4c5c([2H])c([2H])c([2H])c([2H])c5n(-c5ccc(-c6nc(-c7ccccc7)nc(-c7cccc8oc9ccccc9c78)n6)c(-c6ccccc6)c5)c34)c2c1]

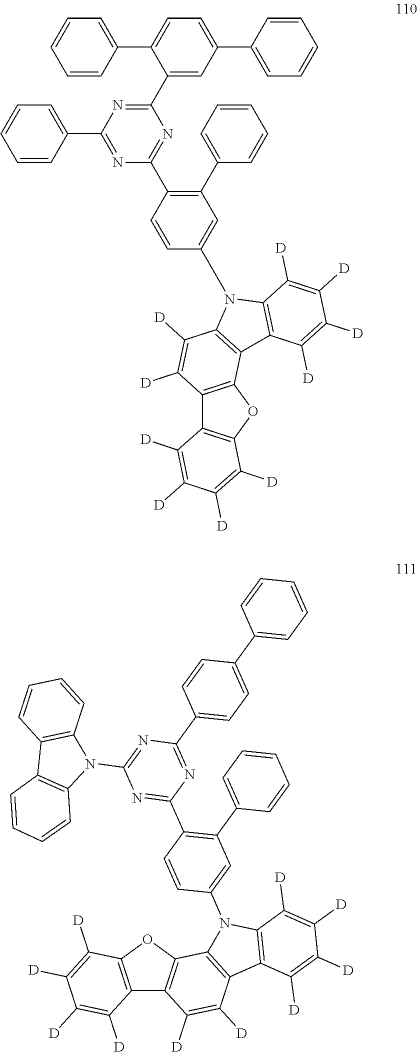 [SMILES: [2H]c1c([2H])c([2H])c2c(oc3c2c([2H])c([2H])c2c3c3c([2H])c([2H])c([2H])c([2H])c3n2-c2ccc(-c3nc(-c4ccccc4)nc(-c4cc(-c5ccccc5)ccc4-c4ccccc4)n3)c(-c3ccccc3)c2)c1[2H] ; [2H]c1c([2H])c([2H])c2c(oc3c2c([2H])c([2H])c2c4c([2H])c([2H])c([2H])c([2H])c4n(-c4ccc(-c5nc(-c6ccc(-c7ccccc7)cc6)nc(-n6c7ccccc7c7ccccc76)n5)c(-c5ccccc5)c4)c32)c1[2H]]

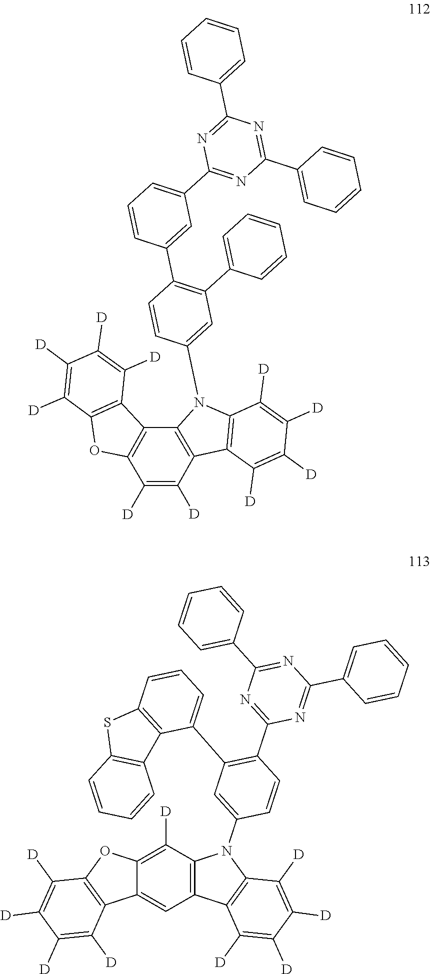 [SMILES: [2H]c1c([2H])c([2H])c2c(oc3c([2H])c([2H])c4c5c([2H])c([2H])c([2H])c([2H])c5n(-c5ccc(-c6cccc(-c7nc(-c8ccccc8)nc(-c8ccccc8)n7)c6)c(-c6ccccc6)c5)c4c32)c1[2H] ; [2H]c1c([2H])c([2H])c2c(oc3c([2H])c4c(cc32)c2c([2H])c([2H])c([2H])c([2H])c2n4-c2ccc(-c3nc(-c4ccccc4)nc(-c4ccccc4)n3)c(-c3cccc4sc5ccccc5c34)c2)c1[2H]]

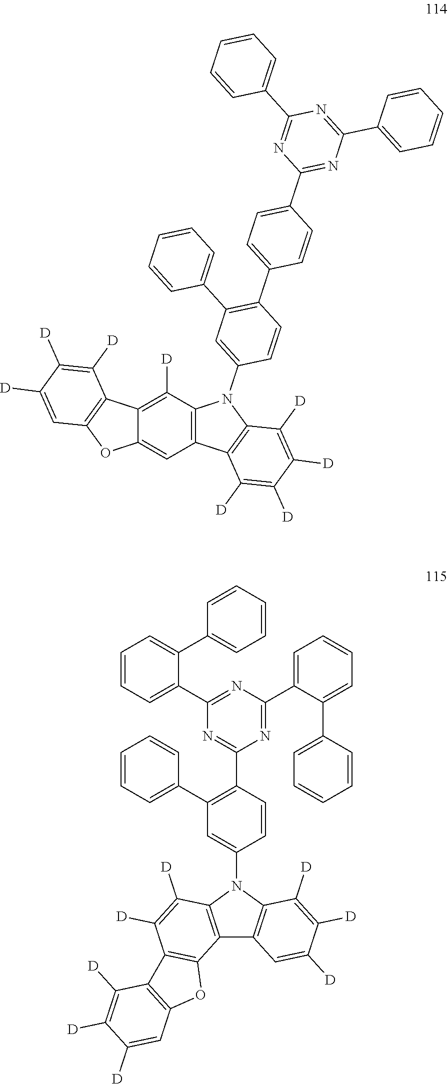 [SMILES: [2H]c1cc2oc3cc4c5c([2H])c([2H])c([2H])c([2H])c5n(-c5ccc(-c6ccc(-c7nc(-c8ccccc8)nc(-c8ccccc8)n7)cc6)c(-c6ccccc6)c5)c4c([2H])c3c2c([2H])c1[2H] ; [2H]c1cc2oc3c(c([2H])c([2H])c4c3c3cc([2H])c([2H])c([2H])c3n4-c3ccc(-c4nc(-c5ccccc5-c5ccccc5)nc(-c5ccccc5-c5ccccc5)n4)c(-c4ccccc4)c3)c2c([2H])c1[2H]]

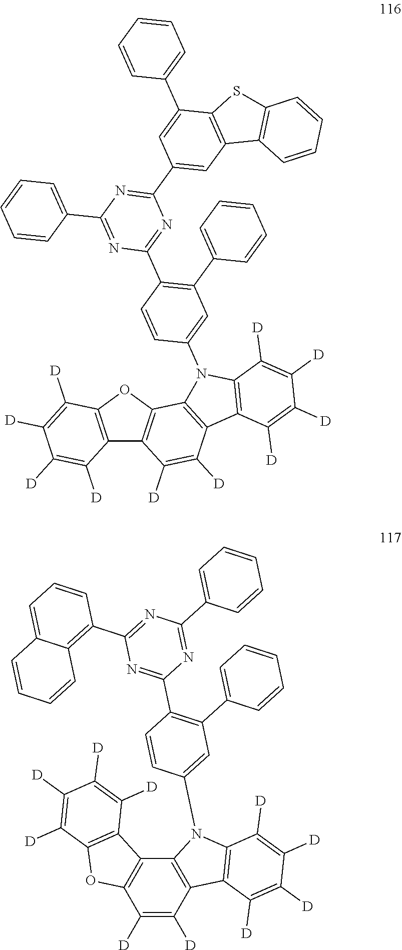 [SMILES: [2H]c1c([2H])c([2H])c2c(oc3c2c([2H])c([2H])c2c4c([2H])c([2H])c([2H])c([2H])c4n(-c4ccc(-c5nc(-c6ccccc6)nc(-c6cc(-c7ccccc7)c7sc8ccccc8c7c6)n5)c(-c5ccccc5)c4)c32)c1[2H] ; [2H]c1c([2H])c([2H])c2c(oc3c([2H])c([2H])c4c5c([2H])c([2H])c([2H])c([2H])c5n(-c5ccc(-c6nc(-c7ccccc7)nc(-c7cccc8ccccc78)n6)c(-c6ccccc6)c5)c4c32)c1[2H]]

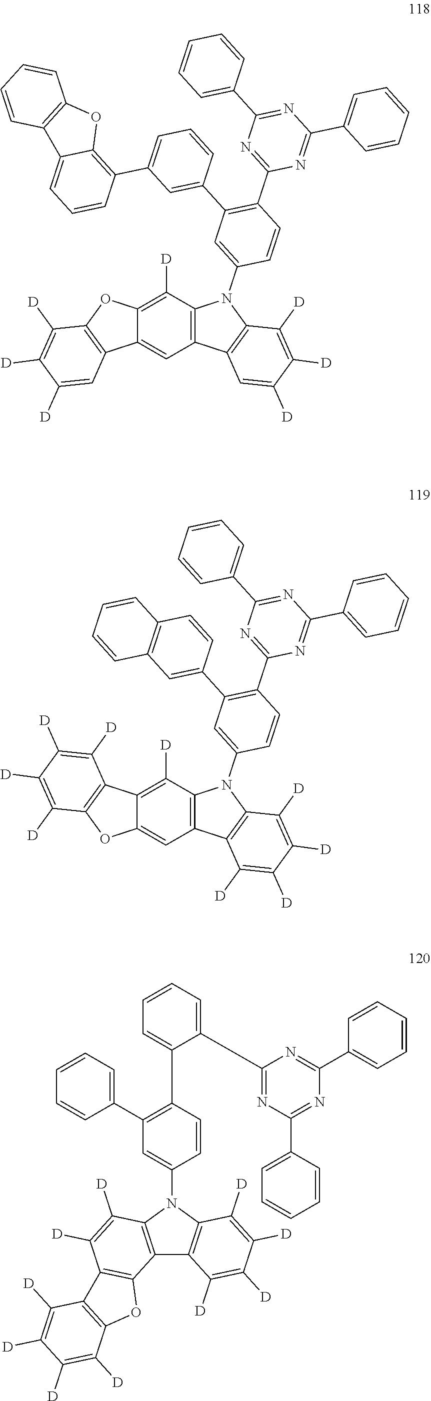 [SMILES: [2H]c1cc2c(oc3c([2H])c4c(cc32)c2cc([2H])c([2H])c([2H])c2n4-c2ccc(-c3nc(-c4ccccc4)nc(-c4ccccc4)n3)c(-c3cccc(-c4cccc5c4oc4ccccc45)c3)c2)c([2H])c1[2H] ; [2H]c1c([2H])c([2H])c2c(oc3cc4c5c([2H])c([2H])c([2H])c([2H])c5n(-c5ccc(-c6nc(-c7ccccc7)nc(-c7ccccc7)n6)c(-c6ccc7ccccc7c6)c5)c4c([2H])c32)c1[2H] ; [2H]c1c([2H])c([2H])c2c(oc3c2c([2H])c([2H])c2c3c3c([2H])c([2H])c([2H])c([2H])c3n2-c2ccc(-c3ccccc3-c3nc(-c4ccccc4)nc(-c4ccccc4)n3)c(-c3ccccc3)c2)c1[2H]]

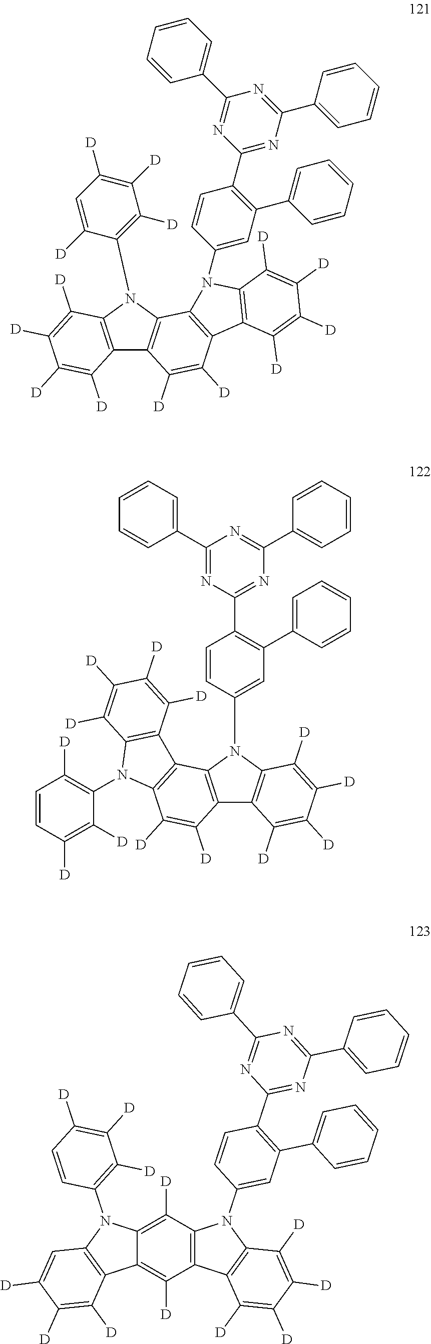

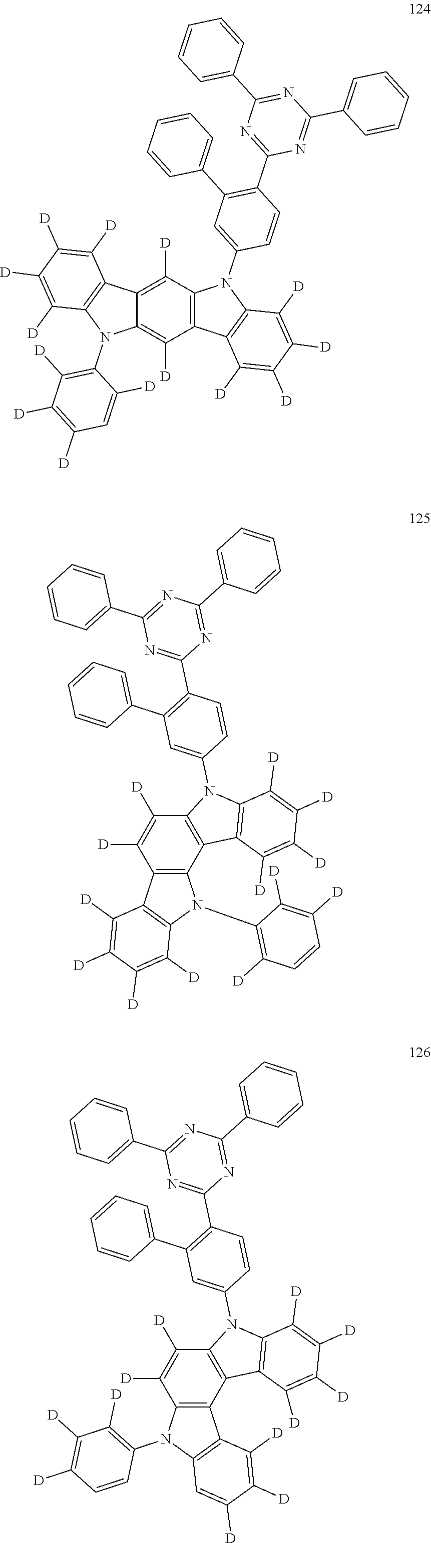

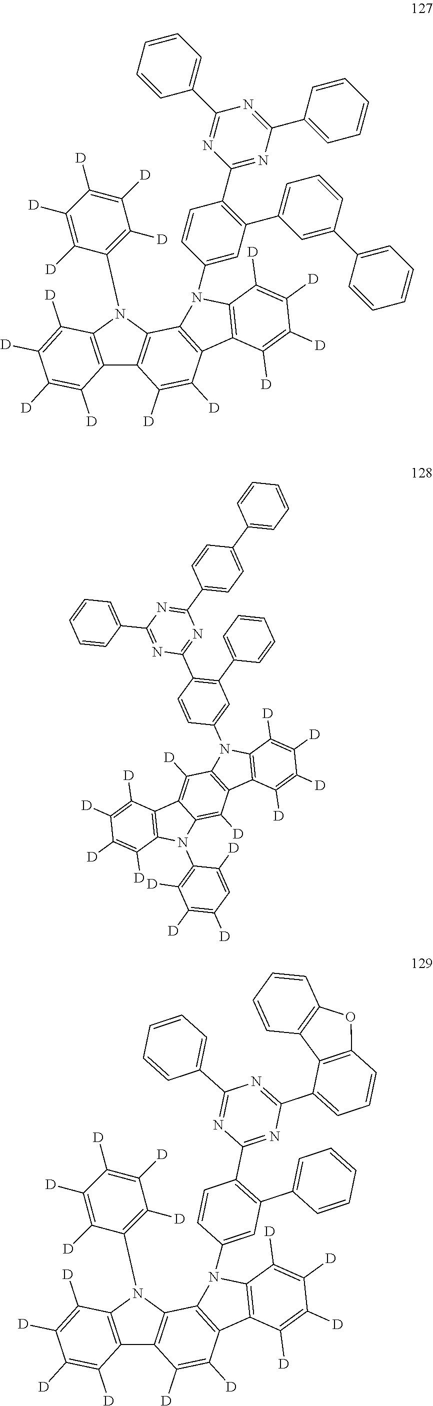

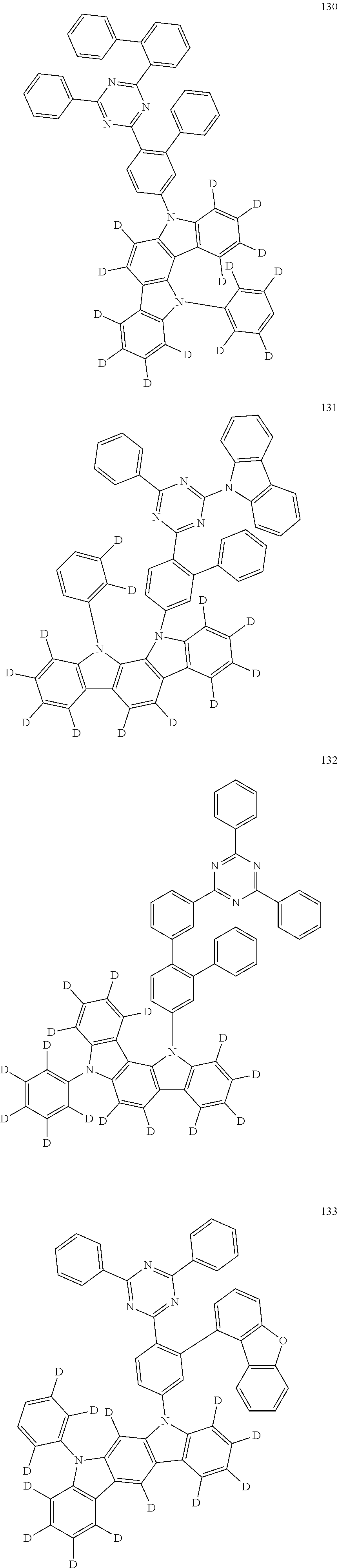

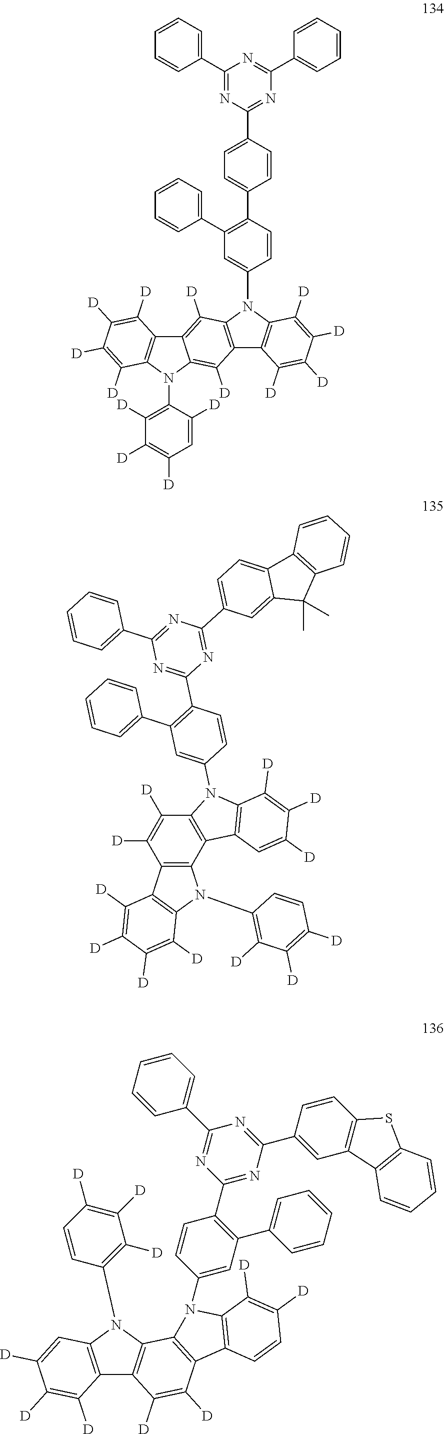

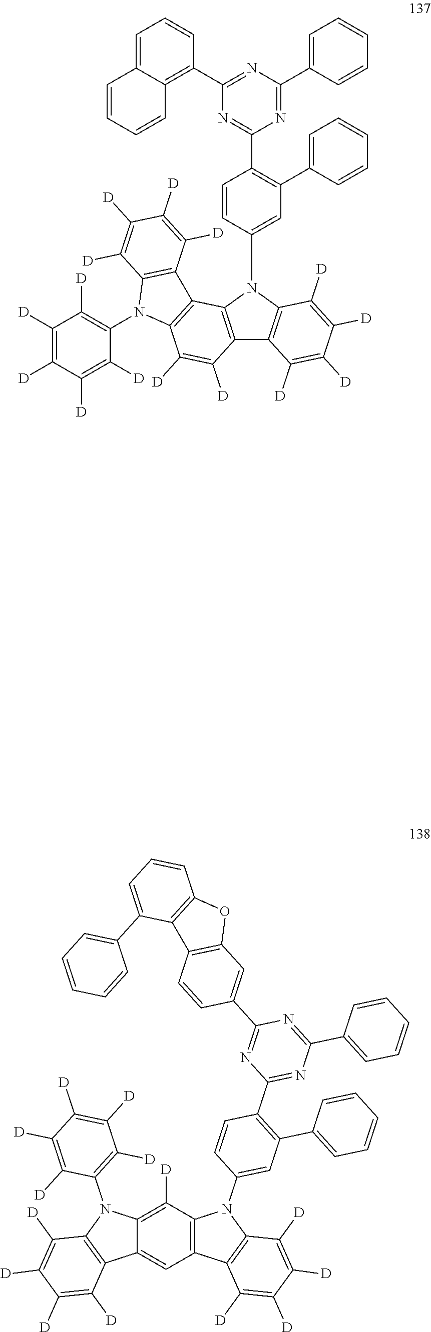 [SMILES: [2H]c1c([2H])c([2H])c(-n2c3c([2H])c([2H])c([2H])c([2H])c3c3c2c([2H])c([2H])c2c4c([2H])c([2H])c([2H])c([2H])c4n(-c4ccc(-c5nc(-c6ccccc6)nc(-c6cccc7ccccc67)n5)c(-c5ccccc5)c4)c23)c([2H])c1[2H] ; [2H]c1c([2H])c([2H])c(-n2c3c([2H])c4c(cc3c3c([2H])c([2H])c([2H])c([2H])c32)c2c([2H])c([2H])c([2H])c([2H])c2n4-c2ccc(-c3nc(-c4ccccc4)nc(-c4ccc5c(c4)oc4cccc(-c6ccccc6)c45)n3)c(-c3ccccc3)c2)c([2H])c1[2H]]

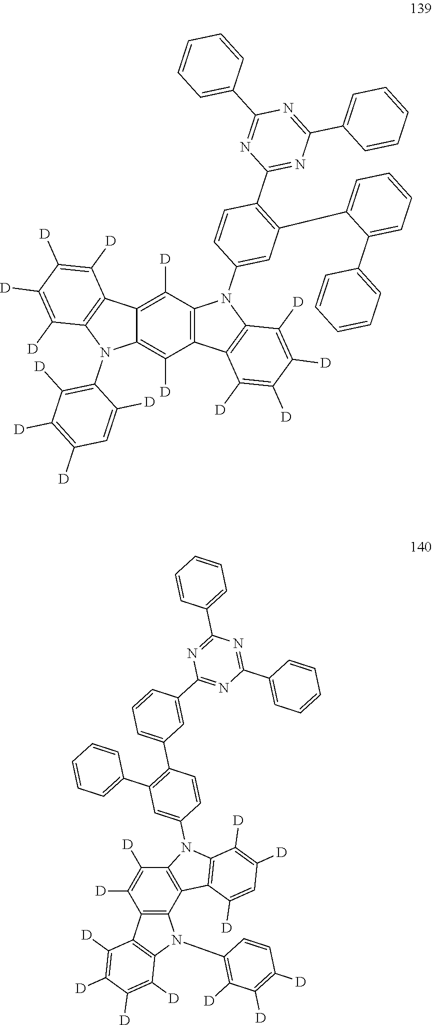 [SMILES: [2H]c1cc([2H])c(-n2c3c([2H])c([2H])c([2H])c([2H])c3c3c([2H])c4c(c([2H])c32)c2c([2H])c([2H])c([2H])c([2H])c2n4-c2ccc(-c3nc(-c4ccccc4)nc(-c4ccccc4)n3)c(-c3ccccc3-c3ccccc3)c2)c([2H])c1[2H] ; [2H]c1ccc(-n2c3c([2H])c([2H])c([2H])c([2H])c3c3c([2H])c([2H])c4c(c5c([2H])cc([2H])c([2H])c5n4-c4ccc(-c5cccc(-c6nc(-c7ccccc7)nc(-c7ccccc7)n6)c5)c(-c5ccccc5)c4)c32)c([2H])c1[2H]]

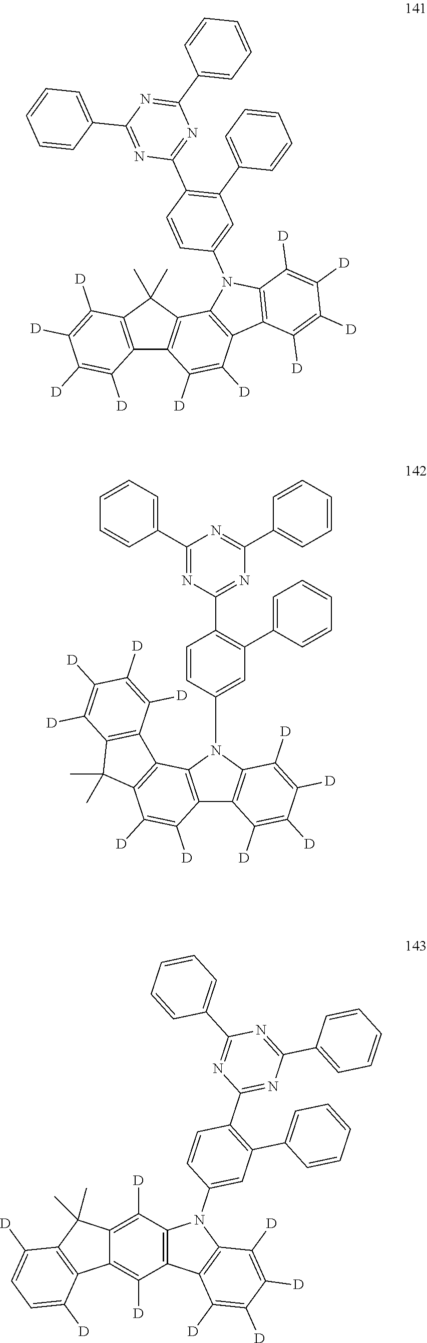 [SMILES: [2H]c1c([2H])c([2H])c2c(c1[2H])-c1c([2H])c([2H])c3c4c([2H])c([2H])c([2H])c([2H])c4n(-c4ccc(-c5nc(-c6ccccc6)nc(-c6ccccc6)n5)c(-c5ccccc5)c4)c3c1C2(C)C ; [2H]c1c([2H])c([2H])c2c(c1[2H])-c1c(c([2H])c([2H])c3c4c([2H])c([2H])c([2H])c([2H])c4n(-c4ccc(-c5nc(-c6ccccc6)nc(-c6ccccc6)n5)c(-c5ccccc5)c4)c13)C2(C)C ; [2H]c1ccc([2H])c2c1-c1c(c([2H])c3c(c1[2H])c1c([2H])c([2H])c([2H])c([2H])c1n3-c1ccc(-c3nc(-c4ccccc4)nc(-c4ccccc4)n3)c(-c3ccccc3)c1)C2(C)C]

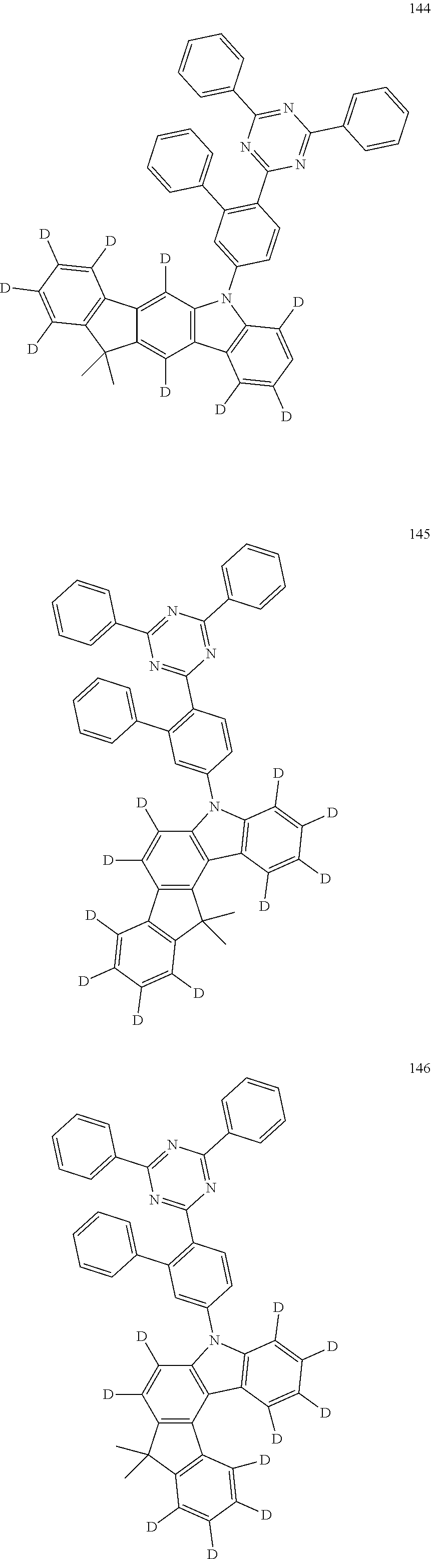 [SMILES: [2H]c1cc([2H])c2c(c1[2H])c1c([2H])c3c(c([2H])c1n2-c1ccc(-c2nc(-c4ccccc4)nc(-c4ccccc4)n2)c(-c2ccccc2)c1)-c1c([2H])c([2H])c([2H])c([2H])c1C3(C)C ; [2H]c1c([2H])c([2H])c2c(c1[2H])-c1c([2H])c([2H])c3c(c1C2(C)C)c1c([2H])c([2H])c([2H])c([2H])c1n3-c1ccc(-c2nc(-c3ccccc3)nc(-c3ccccc3)n2)c(-c2ccccc2)c1 ; [2H]c1c([2H])c([2H])c2c(c1[2H])-c1c(c([2H])c([2H])c3c1c1c([2H])c([2H])c([2H])c([2H])c1n3-c1ccc(-c3nc(-c4ccccc4)nc(-c4ccccc4)n3)c(-c3ccccc3)c1)C2(C)C]

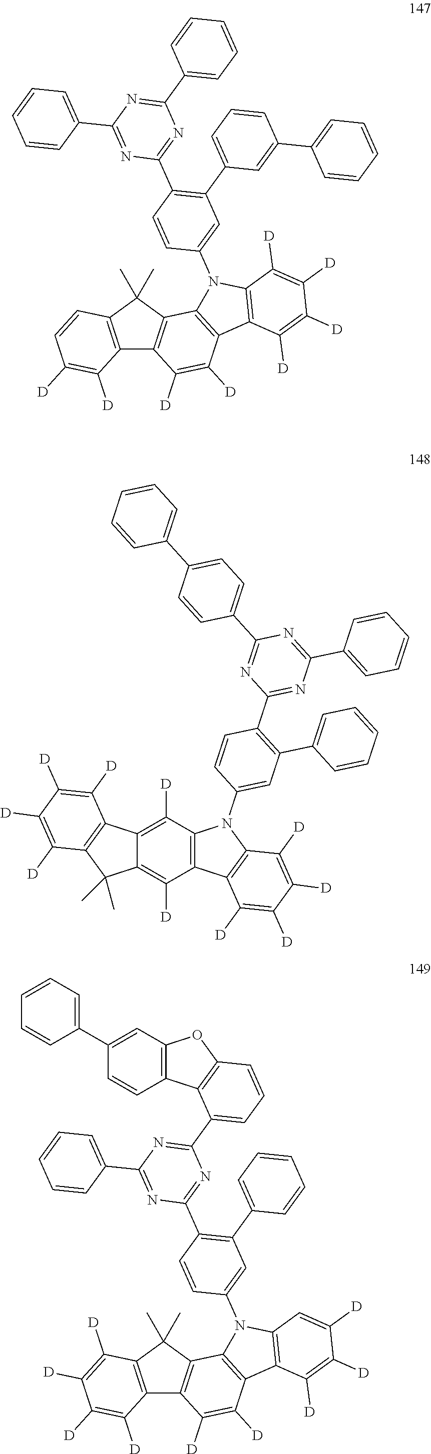 [SMILES: [2H]c1ccc2c(c1[2H])-c1c([2H])c([2H])c3c4c([2H])c([2H])c([2H])c([2H])c4n(-c4ccc(-c5nc(-c6ccccc6)nc(-c6ccccc6)n5)c(-c5cccc(-c6ccccc6)c5)c4)c3c1C2(C)C ; [2H]c1c([2H])c([2H])c2c(c1[2H])-c1c(c([2H])c3c4c([2H])c([2H])c([2H])c([2H])c4n(-c4ccc(-c5nc(-c6ccccc6)nc(-c6ccc(-c7ccccc7)cc6)n5)c(-c5ccccc5)c4)c3c1[2H])C2(C)C ; [2H]c1cc2c(c([2H])c1[2H])c1c([2H])c([2H])c3c(c1n2-c1ccc(-c2nc(-c4ccccc4)nc(-c4cccc5oc6cc(-c7ccccc7)ccc6c45)n2)c(-c2ccccc2)c1)C(C)(C)c1c([2H])c([2H])c([2H])c([2H])c1-3]

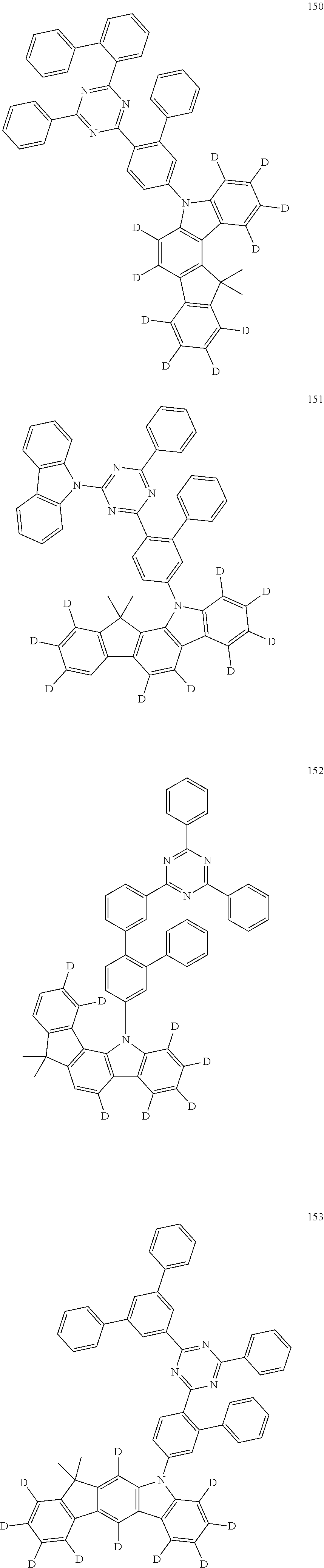

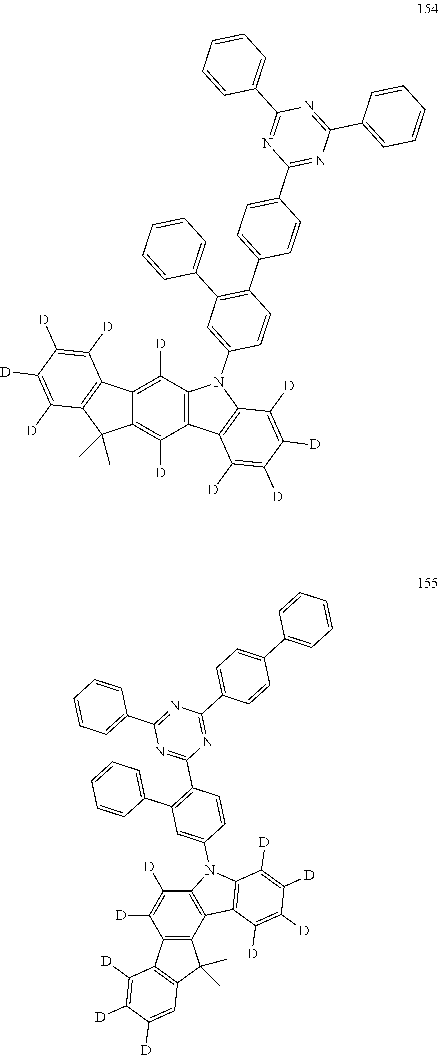 [SMILES: [2H]c1c([2H])c([2H])c2c(c1[2H])-c1c(c([2H])c3c4c([2H])c([2H])c([2H])c([2H])c4n(-c4ccc(-c5ccc(-c6nc(-c7ccccc7)nc(-c7ccccc7)n6)cc5)c(-c5ccccc5)c4)c3c1[2H])C2(C)C ; [2H]c1cc2c(c([2H])c1[2H])-c1c([2H])c([2H])c3c(c1C2(C)C)c1c([2H])c([2H])c([2H])c([2H])c1n3-c1ccc(-c2nc(-c3ccccc3)nc(-c3ccc(-c4ccccc4)cc3)n2)c(-c2ccccc2)c1]

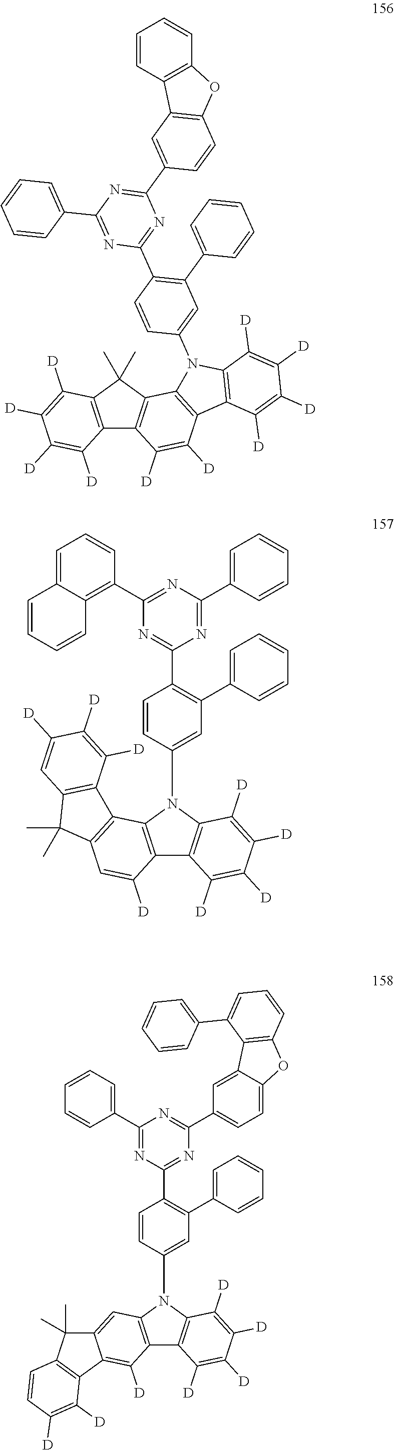 [SMILES: [2H]c1c([2H])c([2H])c2c(c1[2H])-c1c([2H])c([2H])c3c4c([2H])c([2H])c([2H])c([2H])c4n(-c4ccc(-c5nc(-c6ccccc6)nc(-c6ccc7oc8ccccc8c7c6)n5)c(-c5ccccc5)c4)c3c1C2(C)C ; [2H]c1cc2c(c([2H])c1[2H])-c1c(cc([2H])c3c4c([2H])c([2H])c([2H])c([2H])c4n(-c4ccc(-c5nc(-c6ccccc6)nc(-c6cccc7ccccc67)n5)c(-c5ccccc5)c4)c13)C2(C)C ; [2H]c1ccc2c(c1[2H])-c1c(cc3c(c1[2H])c1c([2H])c([2H])c([2H])c([2H])c1n3-c1ccc(-c3nc(-c4ccccc4)nc(-c4ccc5oc6cccc(-c7ccccc7)c6c5c4)n3)c(-c3ccccc3)c1)C2(C)C]

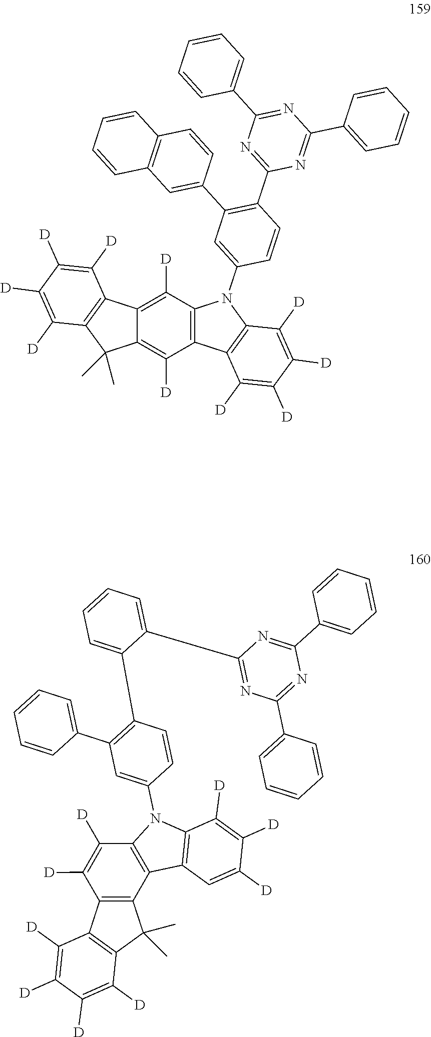 [SMILES: [2H]c1c([2H])c([2H])c2c(c1[2H])-c1c(c([2H])c3c4c([2H])c([2H])c([2H])c([2H])c4n(-c4ccc(-c5nc(-c6ccccc6)nc(-c6ccccc6)n5)c(-c5ccc6ccccc6c5)c4)c3c1[2H])C2(C)C ; [2H]c1cc2c3c4c(c([2H])c([2H])c3n(-c3ccc(-c5ccccc5-c5nc(-c6ccccc6)nc(-c6ccccc6)n5)c(-c5ccccc5)c3)c2c([2H])c1[2H])-c1c([2H])c([2H])c([2H])c([2H])c1C4(C)C]

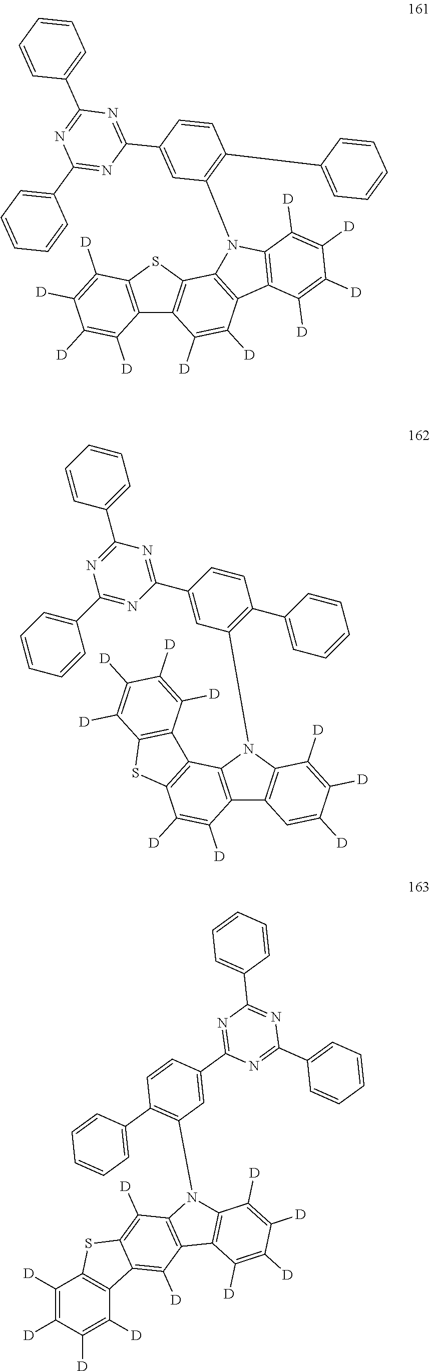 [SMILES: [2H]c1c([2H])c([2H])c2c(sc3c2c([2H])c([2H])c2c4c([2H])c([2H])c([2H])c([2H])c4n(-c4cc(-c5nc(-c6ccccc6)nc(-c6ccccc6)n5)ccc4-c4ccccc4)c32)c1[2H] ; [2H]c1cc2c3c([2H])c([2H])c4sc5c([2H])c([2H])c([2H])c([2H])c5c4c3n(-c3cc(-c4nc(-c5ccccc5)nc(-c5ccccc5)n4)ccc3-c3ccccc3)c2c([2H])c1[2H] ; [2H]c1c([2H])c([2H])c2c(sc3c([2H])c4c(c([2H])c32)c2c([2H])c([2H])c([2H])c([2H])c2n4-c2cc(-c3nc(-c4ccccc4)nc(-c4ccccc4)n3)ccc2-c2ccccc2)c1[2H]]

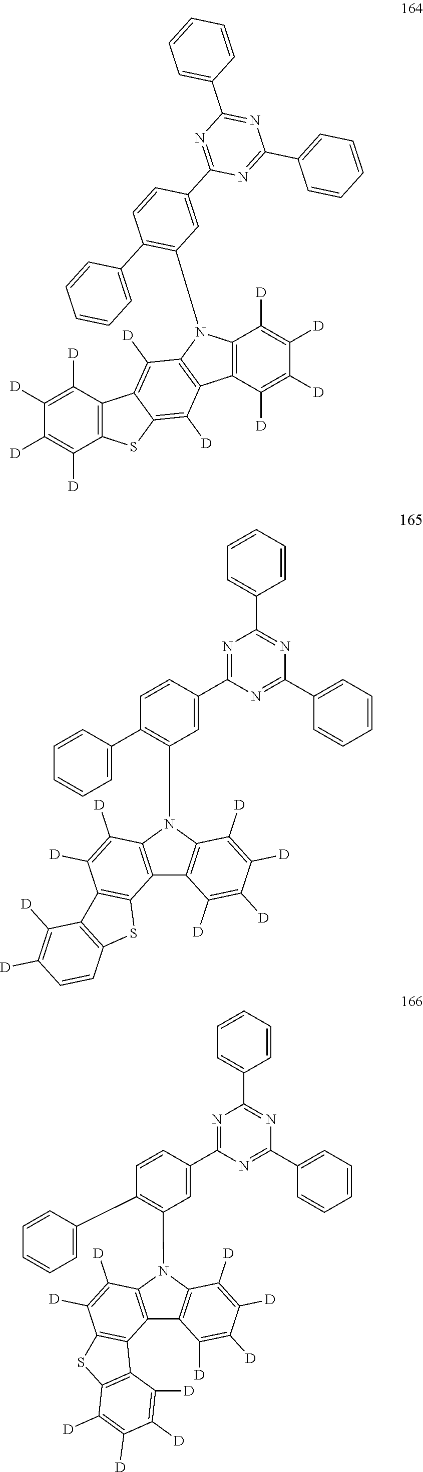 [SMILES: [2H]c1c([2H])c([2H])c2c(sc3c([2H])c4c5c([2H])c([2H])c([2H])c([2H])c5n(-c5cc(-c6nc(-c7ccccc7)nc(-c7ccccc7)n6)ccc5-c5ccccc5)c4c([2H])c32)c1[2H] ; [2H]c1ccc2sc3c(c([2H])c([2H])c4c3c3c([2H])c([2H])c([2H])c([2H])c3n4-c3cc(-c4nc(-c5ccccc5)nc(-c5ccccc5)n4)ccc3-c3ccccc3)c2c1[2H] ; [2H]c1c([2H])c([2H])c2c(sc3c([2H])c([2H])c4c(c5c([2H])c([2H])c([2H])c([2H])c5n4-c4cc(-c5nc(-c6ccccc6)nc(-c6ccccc6)n5)ccc4-c4ccccc4)c32)c1[2H]]

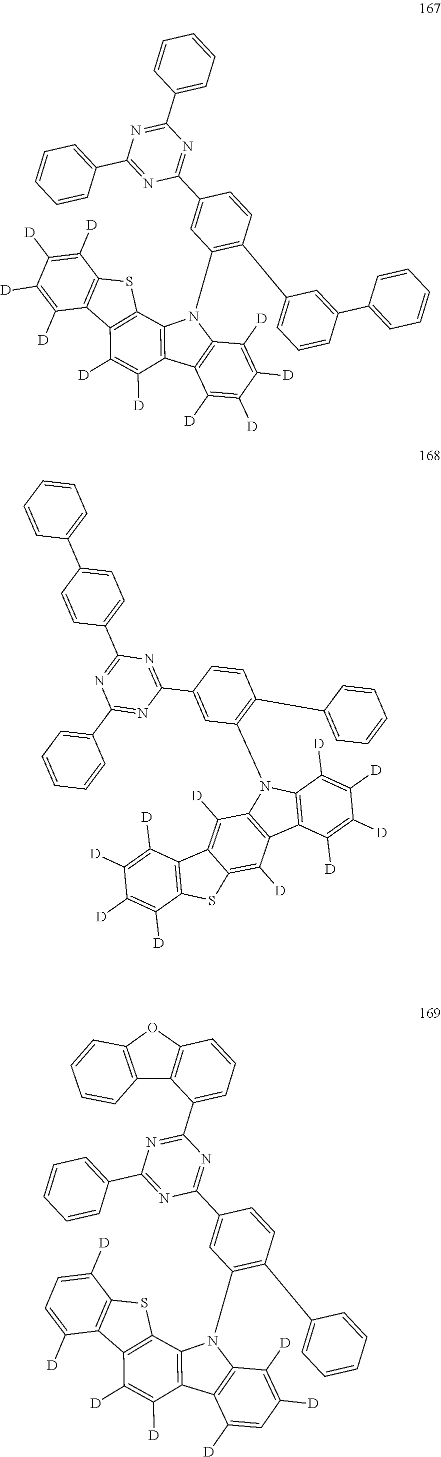 [SMILES: [2H]c1c([2H])c([2H])c2c(sc3c2c([2H])c([2H])c2c4c([2H])c([2H])c([2H])c([2H])c4n(-c4cc(-c5nc(-c6ccccc6)nc(-c6ccccc6)n5)ccc4-c4cccc(-c5ccccc5)c4)c32)c1[2H] ; [2H]c1c([2H])c([2H])c2c(sc3c([2H])c4c5c([2H])c([2H])c([2H])c([2H])c5n(-c5cc(-c6nc(-c7ccccc7)nc(-c7ccc(-c8ccccc8)cc7)n6)ccc5-c5ccccc5)c4c([2H])c32)c1[2H] ; [2H]c1cc([2H])c2c3c([2H])c([2H])c4c(sc5c([2H])ccc([2H])c54)c3n(-c3cc(-c4nc(-c5ccccc5)nc(-c5cccc6oc7ccccc7c56)n4)ccc3-c3ccccc3)c2c1[2H]]

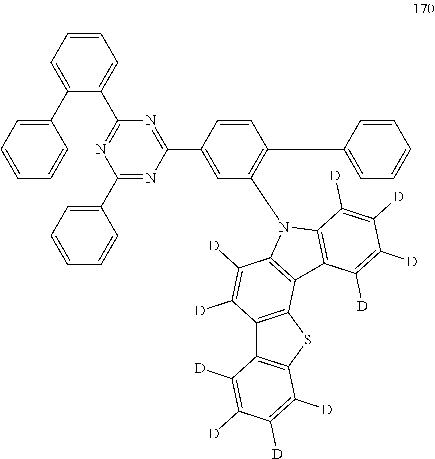 [SMILES: [2H]c1c([2H])c([2H])c2c(sc3c2c([2H])c([2H])c2c3c3c([2H])c([2H])c([2H])c([2H])c3n2-c2cc(-c3nc(-c4ccccc4)nc(-c4ccccc4-c4ccccc4)n3)ccc2-c2ccccc2)c1[2H]]

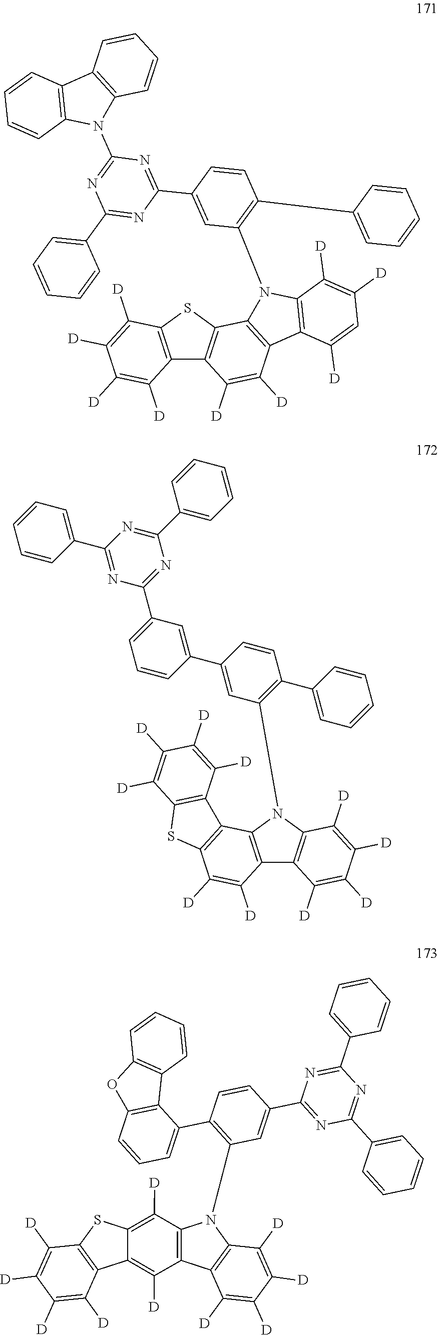 [SMILES: [2H]c1cc([2H])c2c3c([2H])c([2H])c4c(sc5c([2H])c([2H])c([2H])c([2H])c54)c3n(-c3cc(-c4nc(-c5ccccc5)nc(-n5c6ccccc6c6ccccc65)n4)ccc3-c3ccccc3)c2c1[2H] ; [2H]c1c([2H])c([2H])c2c(sc3c([2H])c([2H])c4c5c([2H])c([2H])c([2H])c([2H])c5n(-c5cc(-c6cccc(-c7nc(-c8ccccc8)nc(-c8ccccc8)n7)c6)ccc5-c5ccccc5)c4c32)c1[2H] ; [2H]c1c([2H])c([2H])c2c(sc3c([2H])c4c(c([2H])c32)c2c([2H])c([2H])c([2H])c([2H])c2n4-c2cc(-c3nc(-c4ccccc4)nc(-c4ccccc4)n3)ccc2-c2cccc3oc4ccccc4c23)c1[2H]]

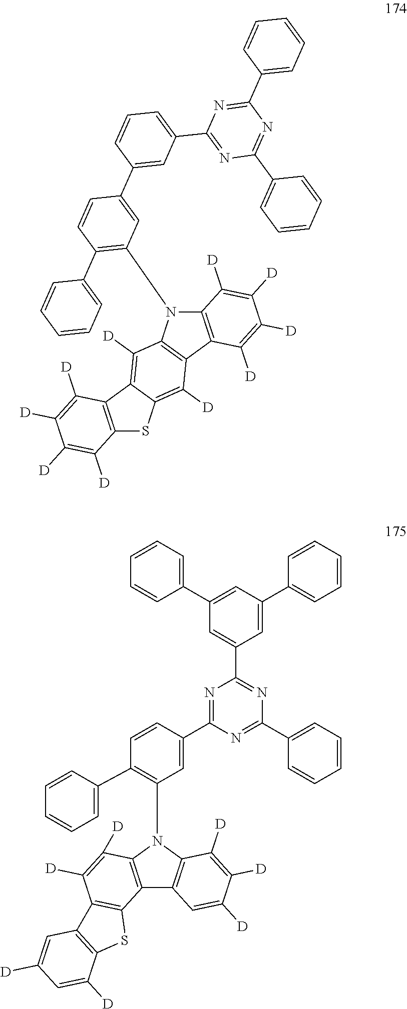 [SMILES: [2H]c1c([2H])c([2H])c2c(sc3c([2H])c4c5c([2H])c([2H])c([2H])c([2H])c5n(-c5cc(-c6cccc(-c7nc(-c8ccccc8)nc(-c8ccccc8)n7)c6)ccc5-c5ccccc5)c4c([2H])c32)c1[2H] ; [2H]c1cc([2H])c2sc3c(c([2H])c([2H])c4c3c3cc([2H])c([2H])c([2H])c3n4-c3cc(-c4nc(-c5ccccc5)nc(-c5cc(-c6ccccc6)cc(-c6ccccc6)c5)n4)ccc3-c3ccccc3)c2c1]

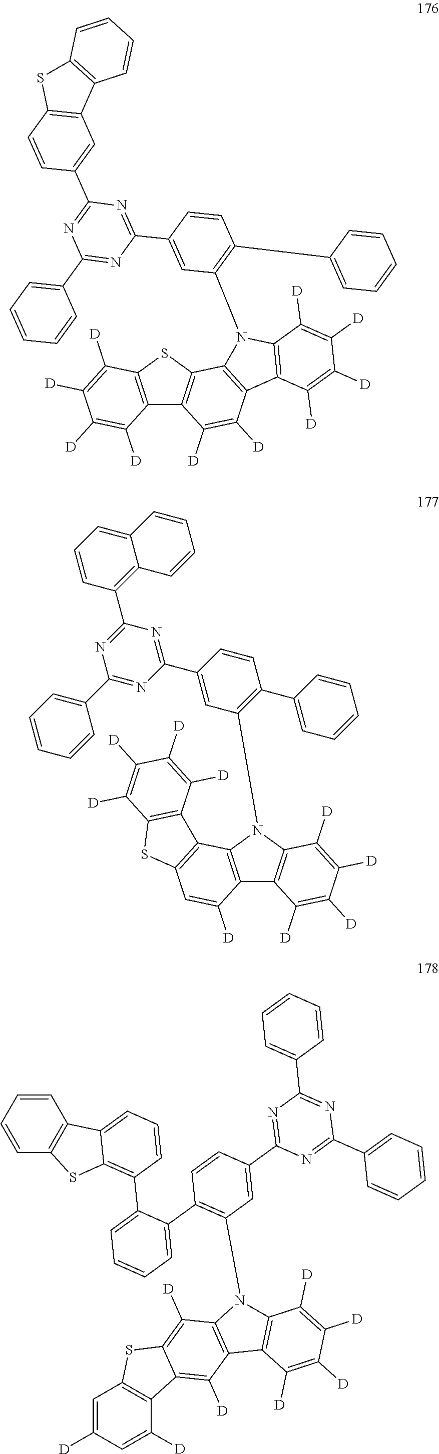 [SMILES: [2H]c1c([2H])c([2H])c2c(sc3c2c([2H])c([2H])c2c4c([2H])c([2H])c([2H])c([2H])c4n(-c4cc(-c5nc(-c6ccccc6)nc(-c6ccc7sc8ccccc8c7c6)n5)ccc4-c4ccccc4)c32)c1[2H] ; [2H]c1c([2H])c([2H])c2c(sc3cc([2H])c4c5c([2H])c([2H])c([2H])c([2H])c5n(-c5cc(-c6nc(-c7ccccc7)nc(-c7cccc8ccccc78)n6)ccc5-c5ccccc5)c4c32)c1[2H] ; [2H]c1cc([2H])c2c(c1)sc1c([2H])c3c(c([2H])c12)c1c([2H])c([2H])c([2H])c([2H])c1n3-c1cc(-c2nc(-c3ccccc3)nc(-c3ccccc3)n2)ccc1-c1ccccc1-c1cccc2c1sc1ccccc12]

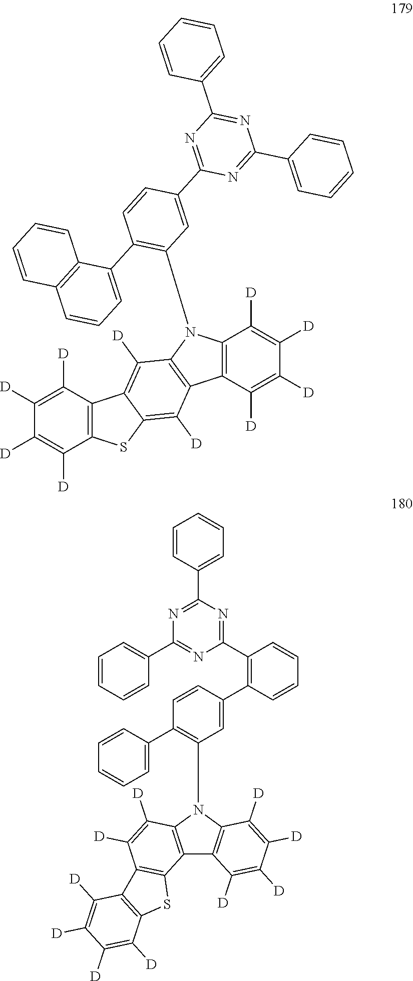 [SMILES: [2H]c1c([2H])c([2H])c2c(sc3c([2H])c4c5c([2H])c([2H])c([2H])c([2H])c5n(-c5cc(-c6nc(-c7ccccc7)nc(-c7ccccc7)n6)ccc5-c5cccc6ccccc56)c4c([2H])c32)c1[2H] ; [2H]c1c([2H])c([2H])c2c(sc3c2c([2H])c([2H])c2c3c3c([2H])c([2H])c([2H])c([2H])c3n2-c2cc(-c3ccccc3-c3nc(-c4ccccc4)nc(-c4ccccc4)n3)ccc2-c2ccccc2)c1[2H]]

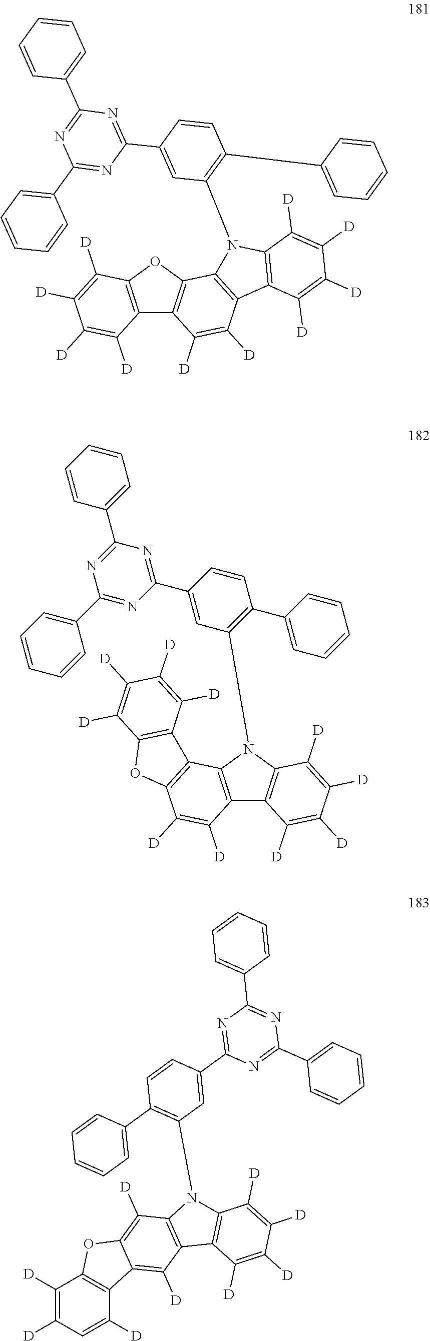 [SMILES: [2H]c1c([2H])c([2H])c2c(oc3c2c([2H])c([2H])c2c4c([2H])c([2H])c([2H])c([2H])c4n(-c4cc(-c5nc(-c6ccccc6)nc(-c6ccccc6)n5)ccc4-c4ccccc4)c32)c1[2H] ; [2H]c1c([2H])c([2H])c2c(oc3c([2H])c([2H])c4c5c([2H])c([2H])c([2H])c([2H])c5n(-c5cc(-c6nc(-c7ccccc7)nc(-c7ccccc7)n6)ccc5-c5ccccc5)c4c32)c1[2H] ; [2H]c1cc([2H])c2c(oc3c([2H])c4c(c([2H])c32)c2c([2H])c([2H])c([2H])c([2H])c2n4-c2cc(-c3nc(-c4ccccc4)nc(-c4ccccc4)n3)ccc2-c2ccccc2)c1[2H]]

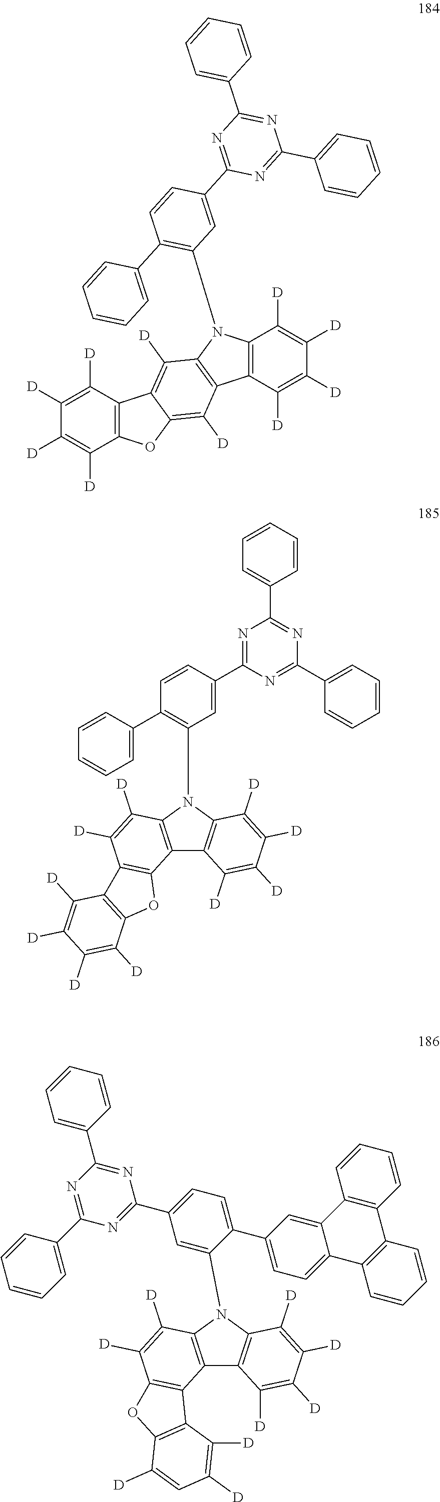 [SMILES: [2H]c1c([2H])c([2H])c2c(oc3c([2H])c4c5c([2H])c([2H])c([2H])c([2H])c5n(-c5cc(-c6nc(-c7ccccc7)nc(-c7ccccc7)n6)ccc5-c5ccccc5)c4c([2H])c32)c1[2H] ; [2H]c1c([2H])c([2H])c2c(oc3c2c([2H])c([2H])c2c3c3c([2H])c([2H])c([2H])c([2H])c3n2-c2cc(-c3nc(-c4ccccc4)nc(-c4ccccc4)n3)ccc2-c2ccccc2)c1[2H] ; [2H]c1cc([2H])c2oc3c([2H])c([2H])c4c(c5c([2H])c([2H])c([2H])c([2H])c5n4-c4cc(-c5nc(-c6ccccc6)nc(-c6ccccc6)n5)ccc4-c4ccc5c6ccccc6c6ccccc6c5c4)c3c2c1[2H]]

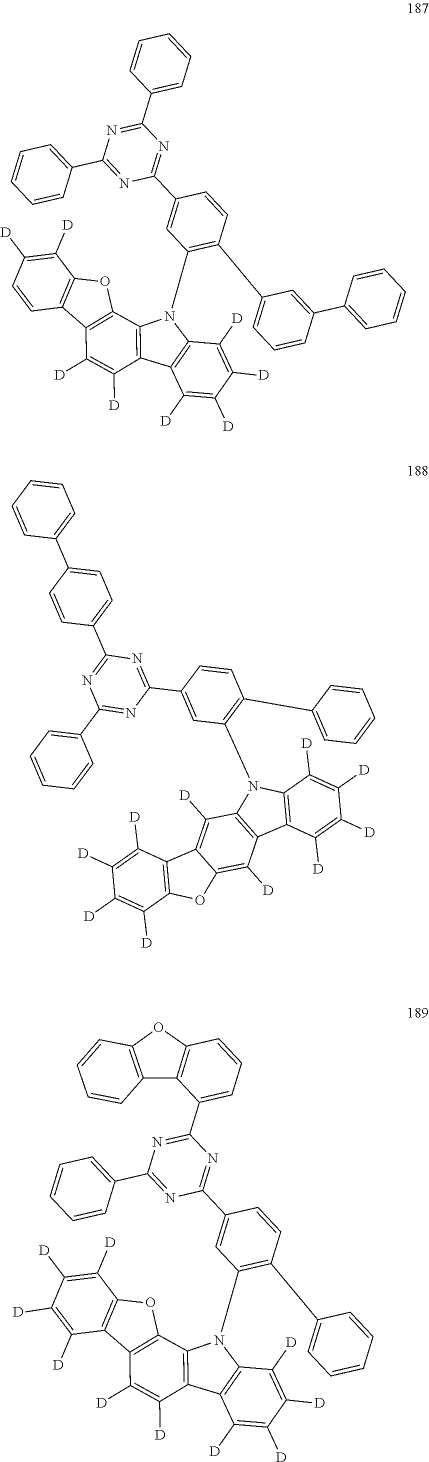 [SMILES: [2H]c1ccc2c(oc3c2c([2H])c([2H])c2c4c([2H])c([2H])c([2H])c([2H])c4n(-c4cc(-c5nc(-c6ccccc6)nc(-c6ccccc6)n5)ccc4-c4cccc(-c5ccccc5)c4)c32)c1[2H] ; [2H]c1c([2H])c([2H])c2c(oc3c([2H])c4c5c([2H])c([2H])c([2H])c([2H])c5n(-c5cc(-c6nc(-c7ccccc7)nc(-c7ccc(-c8ccccc8)cc7)n6)ccc5-c5ccccc5)c4c([2H])c32)c1[2H] ; [2H]c1c([2H])c([2H])c2c(oc3c2c([2H])c([2H])c2c4c([2H])c([2H])c([2H])c([2H])c4n(-c4cc(-c5nc(-c6ccccc6)nc(-c6cccc7oc8ccccc8c67)n5)ccc4-c4ccccc4)c32)c1[2H]]

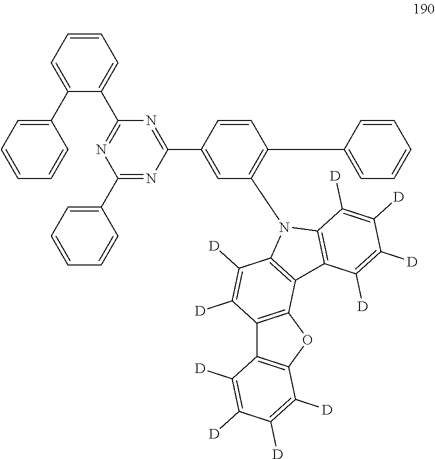 [SMILES: [2H]c1c([2H])c([2H])c2c(oc3c2c([2H])c([2H])c2c3c3c([2H])c([2H])c([2H])c([2H])c3n2-c2cc(-c3nc(-c4ccccc4)nc(-c4ccccc4-c4ccccc4)n3)ccc2-c2ccccc2)c1[2H]]

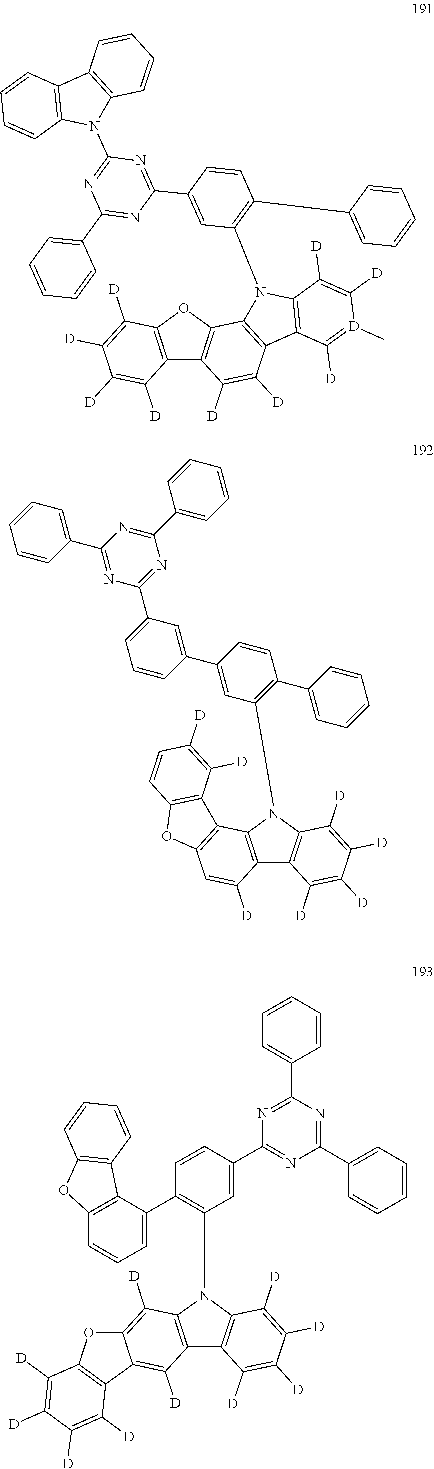

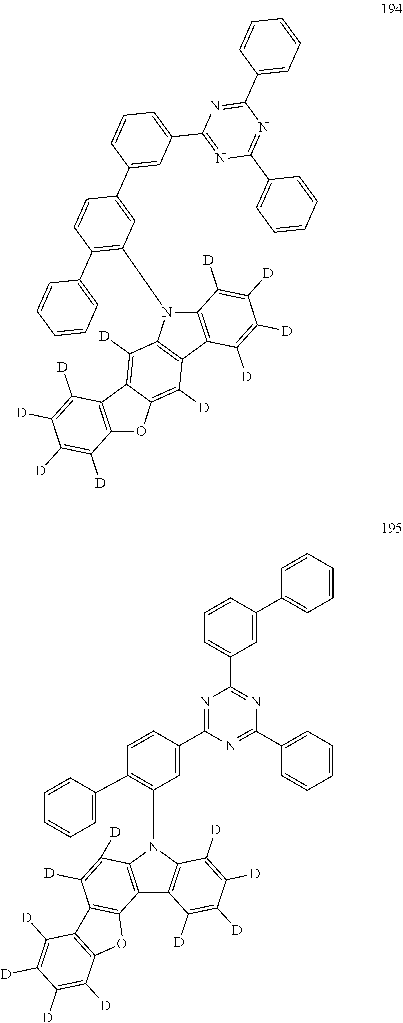 [SMILES: [2H]c1c([2H])c([2H])c2c(oc3c([2H])c4c5c([2H])c([2H])c([2H])c([2H])c5n(-c5cc(-c6cccc(-c7nc(-c8ccccc8)nc(-c8ccccc8)n7)c6)ccc5-c5ccccc5)c4c([2H])c32)c1[2H] ; [2H]c1c([2H])c([2H])c2c(oc3c2c([2H])c([2H])c2c3c3c([2H])c([2H])c([2H])c([2H])c3n2-c2cc(-c3nc(-c4ccccc4)nc(-c4cccc(-c5ccccc5)c4)n3)ccc2-c2ccccc2)c1[2H]]

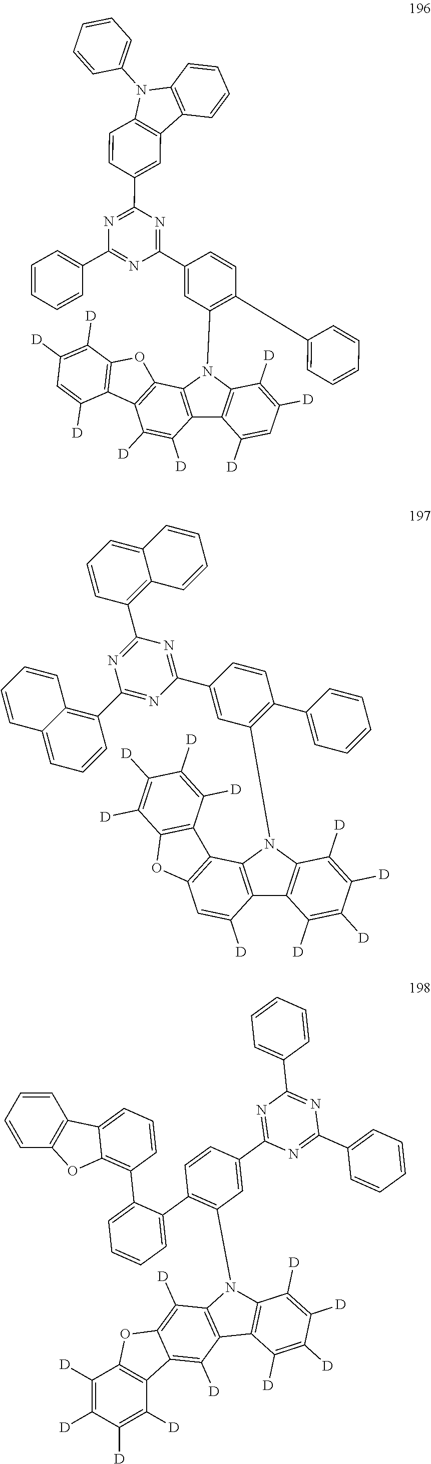 [SMILES: [2H]c1cc([2H])c2c(oc3c2c([2H])c([2H])c2c4c([2H])cc([2H])c([2H])c4n(-c4cc(-c5nc(-c6ccccc6)nc(-c6ccc7c(c6)c6ccccc6n7-c6ccccc6)n5)ccc4-c4ccccc4)c32)c1[2H] ; [2H]c1c([2H])c([2H])c2c(oc3cc([2H])c4c5c([2H])c([2H])c([2H])c([2H])c5n(-c5cc(-c6nc(-c7cccc8ccccc78)nc(-c7cccc8ccccc78)n6)ccc5-c5ccccc5)c4c32)c1[2H] ; [2H]c1c([2H])c([2H])c2c(oc3c([2H])c4c(c([2H])c32)c2c([2H])c([2H])c([2H])c([2H])c2n4-c2cc(-c3nc(-c4ccccc4)nc(-c4ccccc4)n3)ccc2-c2ccccc2-c2cccc3c2oc2ccccc23)c1[2H]]

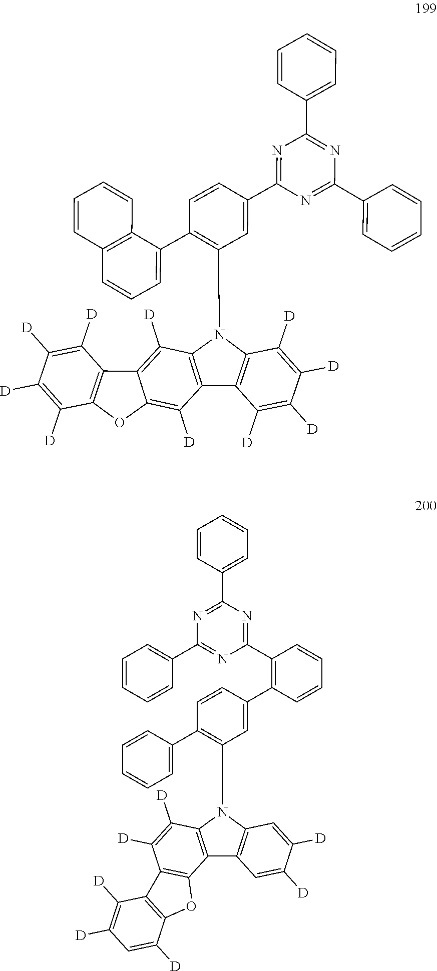 [SMILES: [2H]c1c([2H])c([2H])c2c(oc3c([2H])c4c5c([2H])c([2H])c([2H])c([2H])c5n(-c5cc(-c6nc(-c7ccccc7)nc(-c7ccccc7)n6)ccc5-c5cccc6ccccc56)c4c([2H])c32)c1[2H] ; [2H]c1cc2c3c4oc5c([2H])cc([2H])c([2H])c5c4c([2H])c([2H])c3n(-c3cc(-c4ccccc4-c4nc(-c5ccccc5)nc(-c5ccccc5)n4)ccc3-c3ccccc3)c2cc1[2H]]

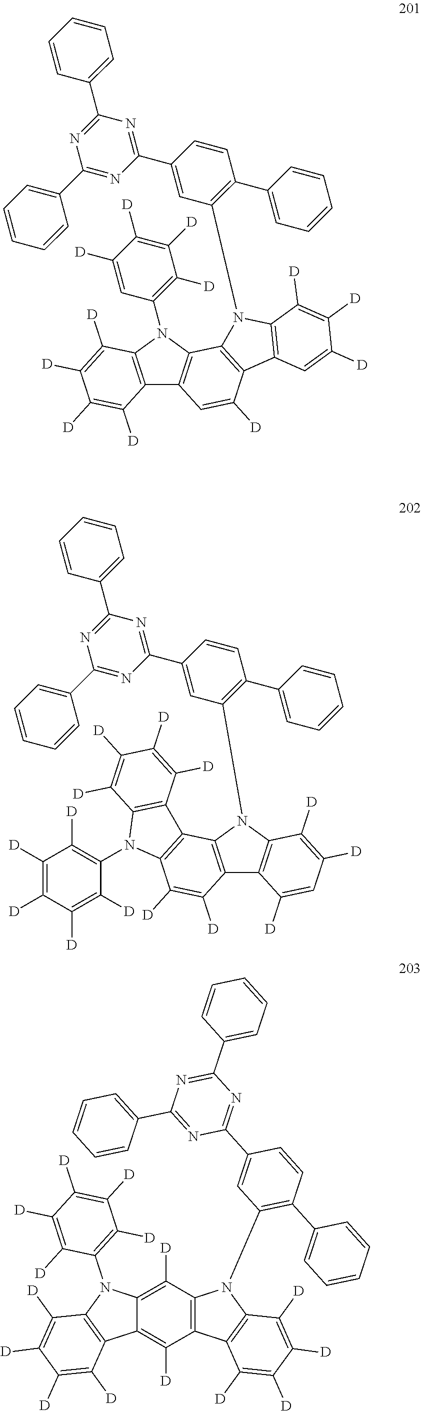

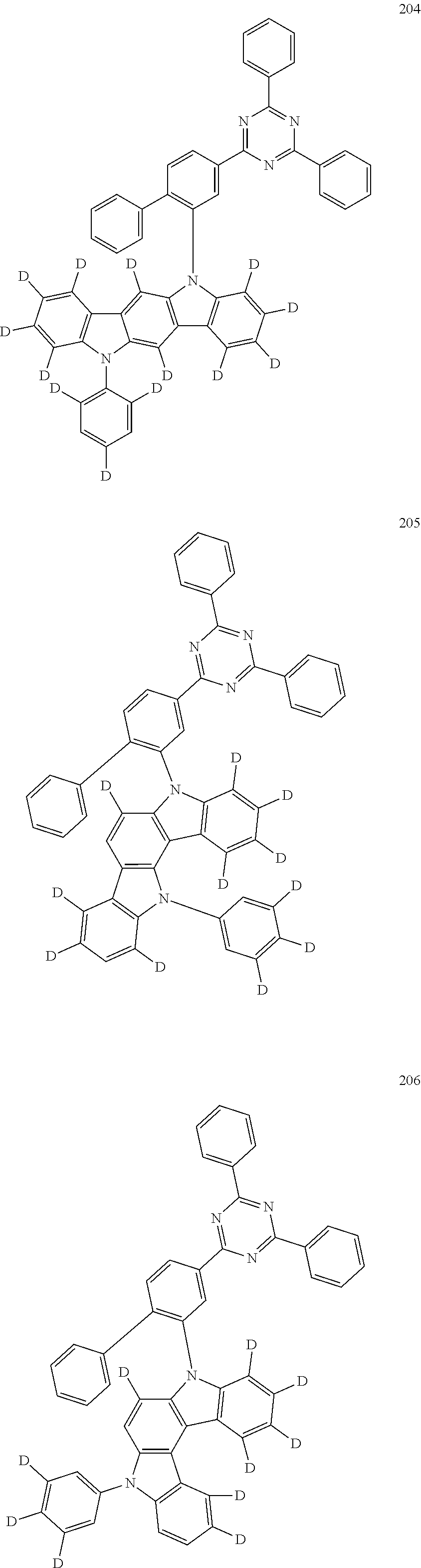 [SMILES: [2H]c1cc([2H])c(-n2c3c([2H])c([2H])c([2H])c([2H])c3c3c([2H])c4c(c([2H])c32)c2c([2H])c([2H])c([2H])c([2H])c2n4-c2cc(-c3nc(-c4ccccc4)nc(-c4ccccc4)n3)ccc2-c2ccccc2)c([2H])c1 ; [2H]c1cc(-n2c3c([2H])cc([2H])c([2H])c3c3cc([2H])c4c(c5c([2H])c([2H])c([2H])c([2H])c5n4-c4cc(-c5nc(-c6ccccc6)nc(-c6ccccc6)n5)ccc4-c4ccccc4)c32)cc([2H])c1[2H] ; [2H]c1cc(-n2c3ccc([2H])c([2H])c3c3c4c5c([2H])c([2H])c([2H])c([2H])c5n(-c5cc(-c6nc(-c7ccccc7)nc(-c7ccccc7)n6)ccc5-c5ccccc5)c4c([2H])cc32)cc([2H])c1[2H]]

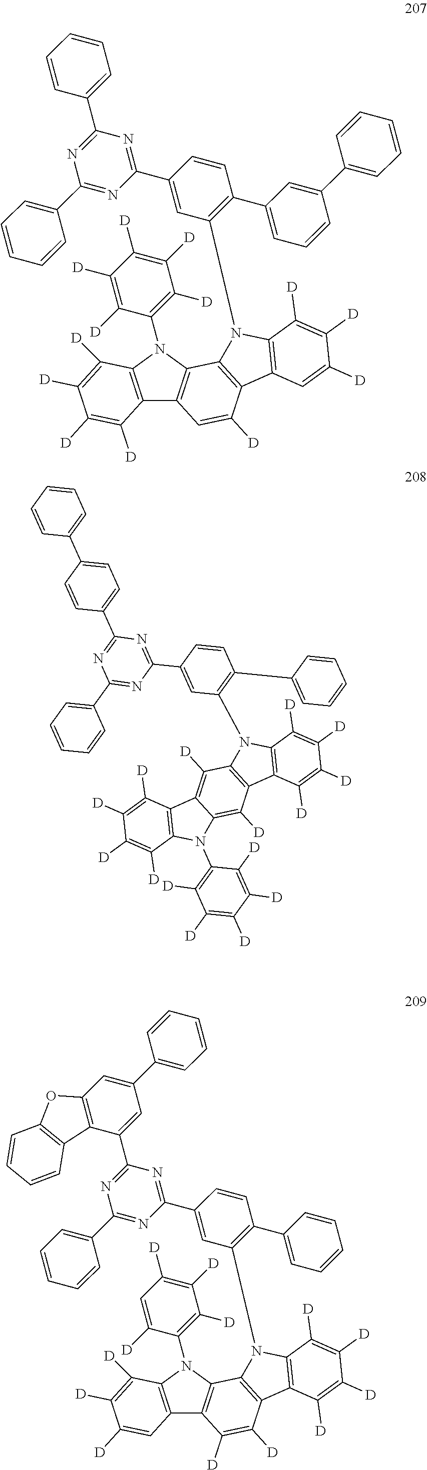

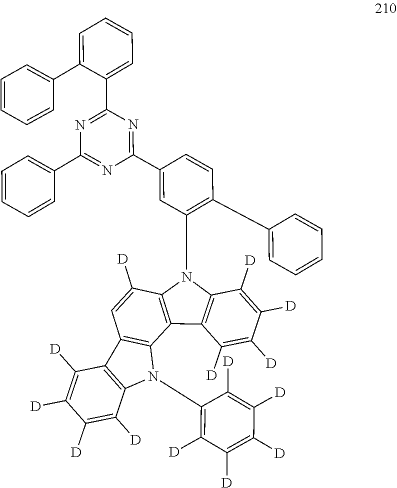 [SMILES: [2H]c1c([2H])c([2H])c(-n2c3c([2H])c([2H])c([2H])c([2H])c3c3cc([2H])c4c(c5c([2H])c([2H])c([2H])c([2H])c5n4-c4cc(-c5nc(-c6ccccc6)nc(-c6ccccc6-c6ccccc6)n5)ccc4-c4ccccc4)c32)c([2H])c1[2H]]

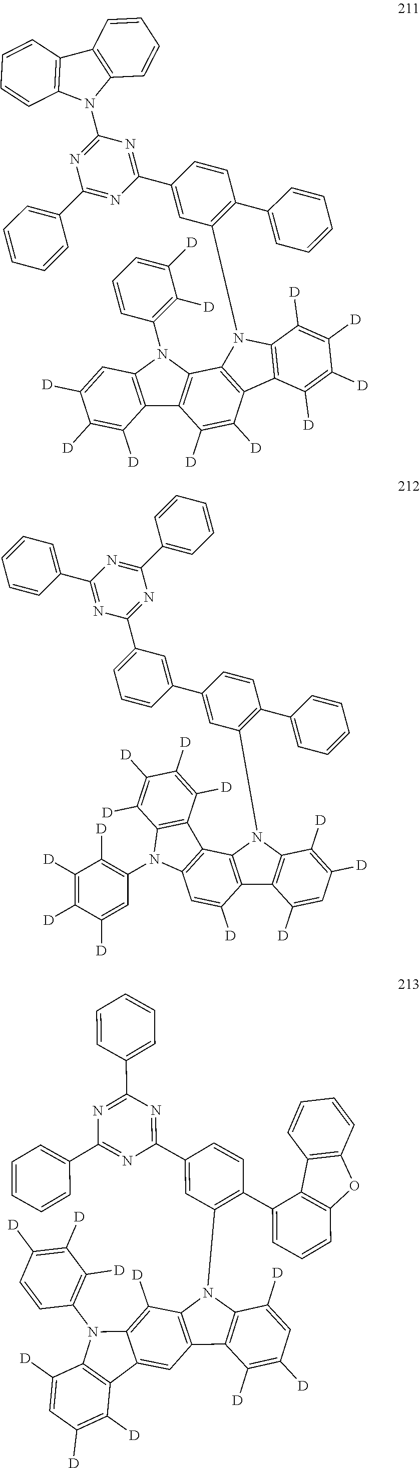

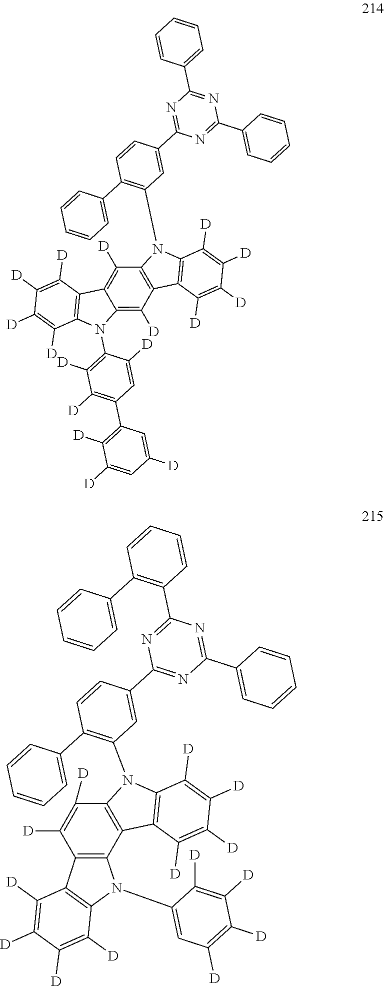 [SMILES: [2H]c1cc([2H])c([2H])c(-c2cc([2H])c(-n3c4c([2H])c([2H])c([2H])c([2H])c4c4c([2H])c5c(c([2H])c43)c3c([2H])c([2H])c([2H])c([2H])c3n5-c3cc(-c4nc(-c5ccccc5)nc(-c5ccccc5)n4)ccc3-c3ccccc3)c([2H])c2[2H])c1 ; [2H]c1cc(-n2c3c([2H])c([2H])c([2H])c([2H])c3c3c([2H])c([2H])c4c(c5c([2H])c([2H])c([2H])c([2H])c5n4-c4cc(-c5nc(-c6ccccc6)nc(-c6ccccc6-c6ccccc6)n5)ccc4-c4ccccc4)c32)c([2H])c([2H])c1[2H]]

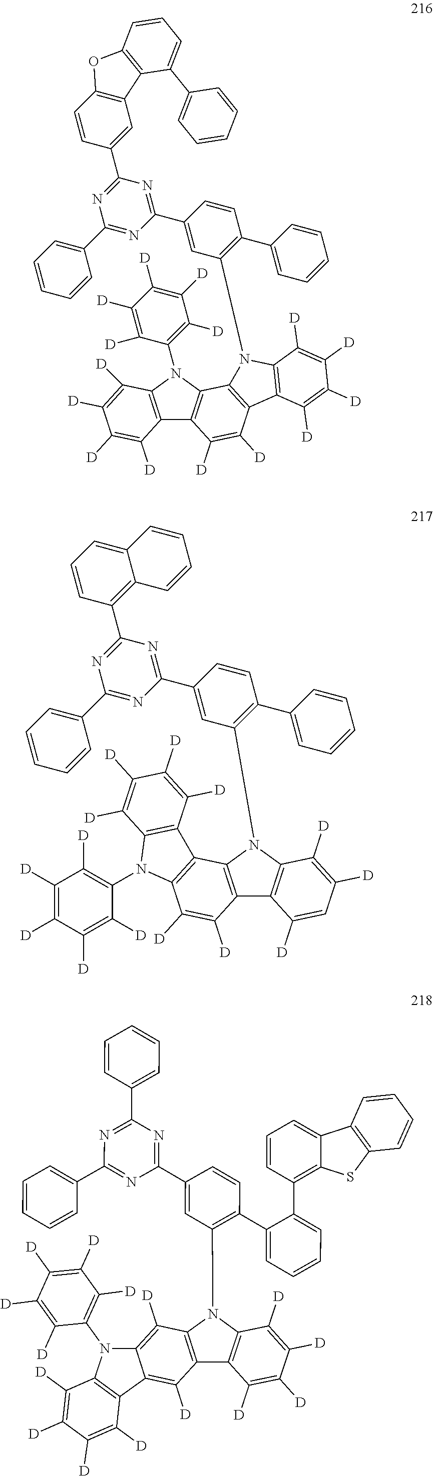

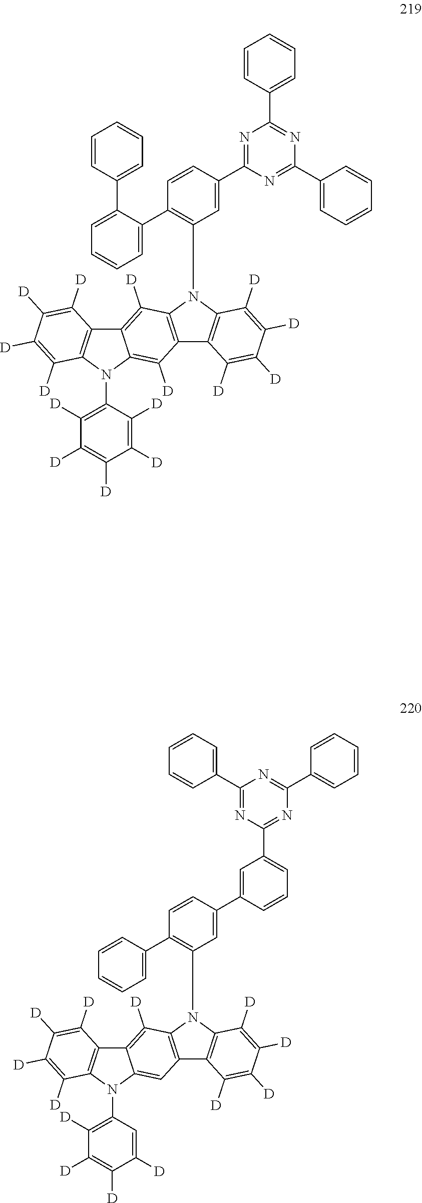 [SMILES: [2H]c1c([2H])c([2H])c(-n2c3c([2H])c([2H])c([2H])c([2H])c3c3c([2H])c4c(c([2H])c32)c2c([2H])c([2H])c([2H])c([2H])c2n4-c2cc(-c3nc(-c4ccccc4)nc(-c4ccccc4)n3)ccc2-c2ccccc2-c2ccccc2)c([2H])c1[2H] ; [2H]c1cc(-n2c3cc4c5c([2H])c([2H])c([2H])c([2H])c5n(-c5cc(-c6cccc(-c7nc(-c8ccccc8)nc(-c8ccccc8)n7)c6)ccc5-c5ccccc5)c4c([2H])c3c3c([2H])c([2H])c([2H])c([2H])c32)c([2H])c([2H])c1[2H]]

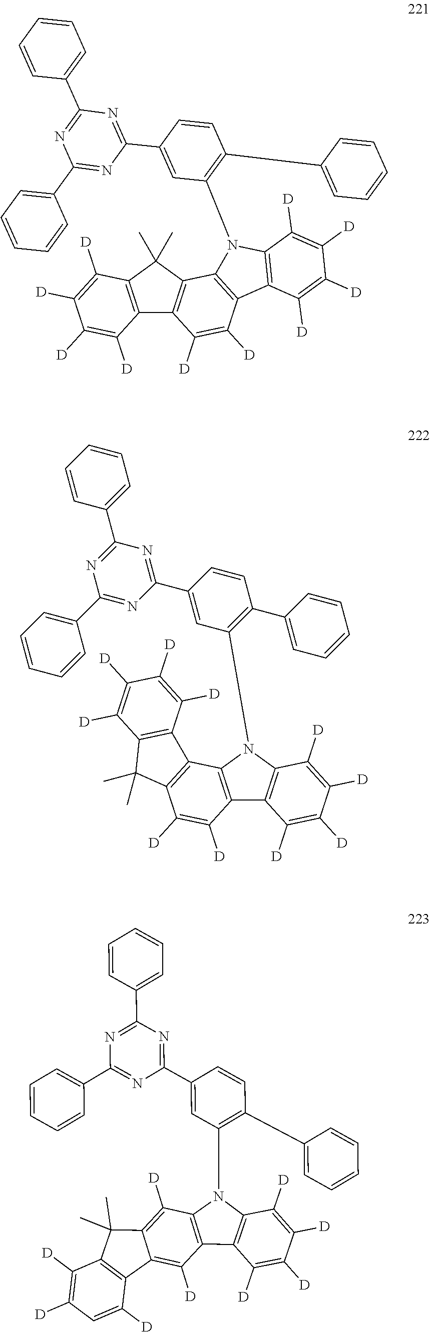 [SMILES: [2H]c1c([2H])c([2H])c2c(c1[2H])-c1c([2H])c([2H])c3c4c([2H])c([2H])c([2H])c([2H])c4n(-c4cc(-c5nc(-c6ccccc6)nc(-c6ccccc6)n5)ccc4-c4ccccc4)c3c1C2(C)C ; [2H]c1c([2H])c([2H])c2c(c1[2H])-c1c(c([2H])c([2H])c3c4c([2H])c([2H])c([2H])c([2H])c4n(-c4cc(-c5nc(-c6ccccc6)nc(-c6ccccc6)n5)ccc4-c4ccccc4)c13)C2(C)C ; [2H]c1cc([2H])c2c(c1[2H])C(C)(C)c1c-2c([2H])c2c3c([2H])c([2H])c([2H])c([2H])c3n(-c3cc(-c4nc(-c5ccccc5)nc(-c5ccccc5)n4)ccc3-c3ccccc3)c2c1[2H]]

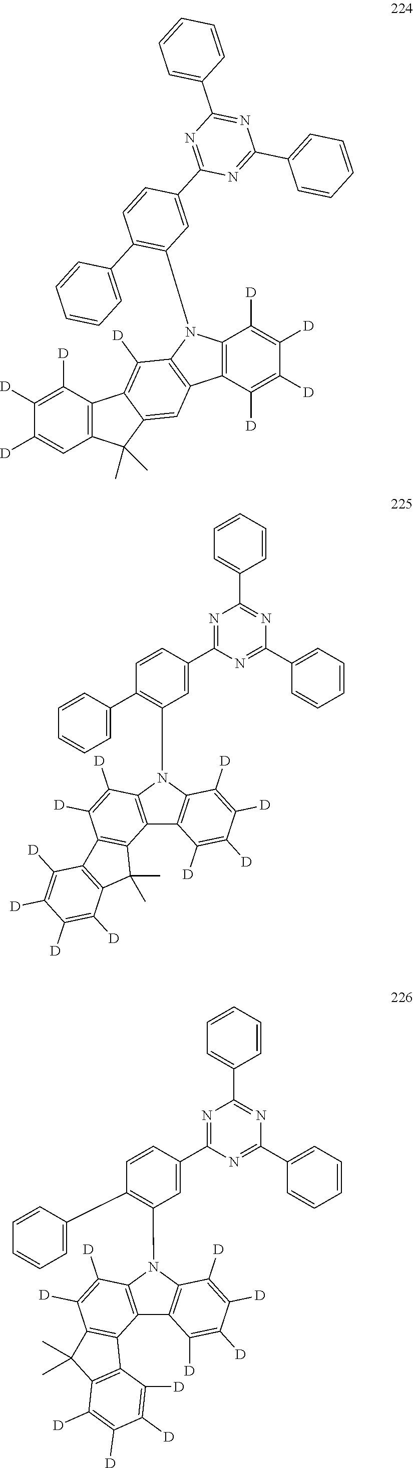 [SMILES: [2H]c1cc2c(c([2H])c1[2H])-c1c(cc3c4c([2H])c([2H])c([2H])c([2H])c4n(-c4cc(-c5nc(-c6ccccc6)nc(-c6ccccc6)n5)ccc4-c4ccccc4)c3c1[2H])C2(C)C ; [2H]c1c([2H])c([2H])c2c(c1[2H])-c1c([2H])c([2H])c3c(c1C2(C)C)c1c([2H])c([2H])c([2H])c([2H])c1n3-c1cc(-c2nc(-c3ccccc3)nc(-c3ccccc3)n2)ccc1-c1ccccc1 ; [2H]c1c([2H])c([2H])c2c(c1[2H])-c1c(c([2H])c([2H])c3c1c1c([2H])c([2H])c([2H])c([2H])c1n3-c1cc(-c3nc(-c4ccccc4)nc(-c4ccccc4)n3)ccc1-c1ccccc1)C2(C)C]

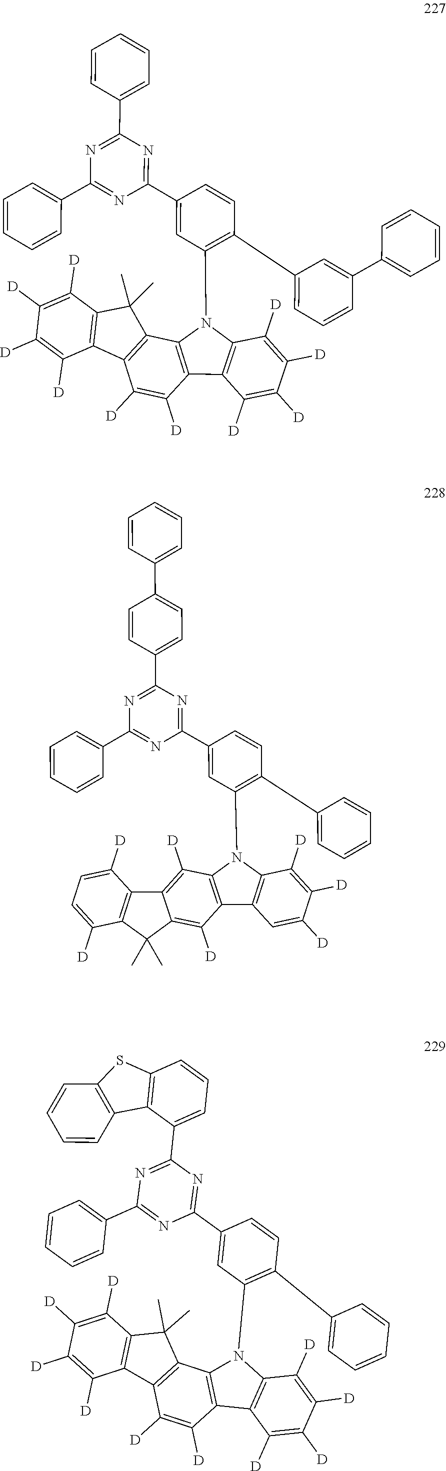 [SMILES: [2H]c1c([2H])c([2H])c2c(c1[2H])-c1c([2H])c([2H])c3c4c([2H])c([2H])c([2H])c([2H])c4n(-c4cc(-c5nc(-c6ccccc6)nc(-c6ccccc6)n5)ccc4-c4cccc(-c5ccccc5)c4)c3c1C2(C)C ; [2H]c1cc2c3c([2H])c4c(c([2H])c3n(-c3cc(-c5nc(-c6ccccc6)nc(-c6ccc(-c7ccccc7)cc6)n5)ccc3-c3ccccc3)c2c([2H])c1[2H])-c1c([2H])ccc([2H])c1C4(C)C ; [2H]c1c([2H])c([2H])c2c(c1[2H])-c1c([2H])c([2H])c3c4c([2H])c([2H])c([2H])c([2H])c4n(-c4cc(-c5nc(-c6ccccc6)nc(-c6cccc7sc8ccccc8c67)n5)ccc4-c4ccccc4)c3c1C2(C)C]

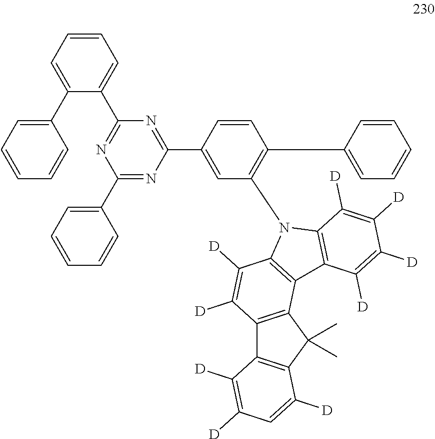 [SMILES: [2H]c1cc([2H])c2c(c1[2H])-c1c([2H])c([2H])c3c(c1C2(C)C)c1c([2H])c([2H])c([2H])c([2H])c1n3-c1cc(-c2nc(-c3ccccc3)nc(-c3ccccc3-c3ccccc3)n2)ccc1-c1ccccc1]

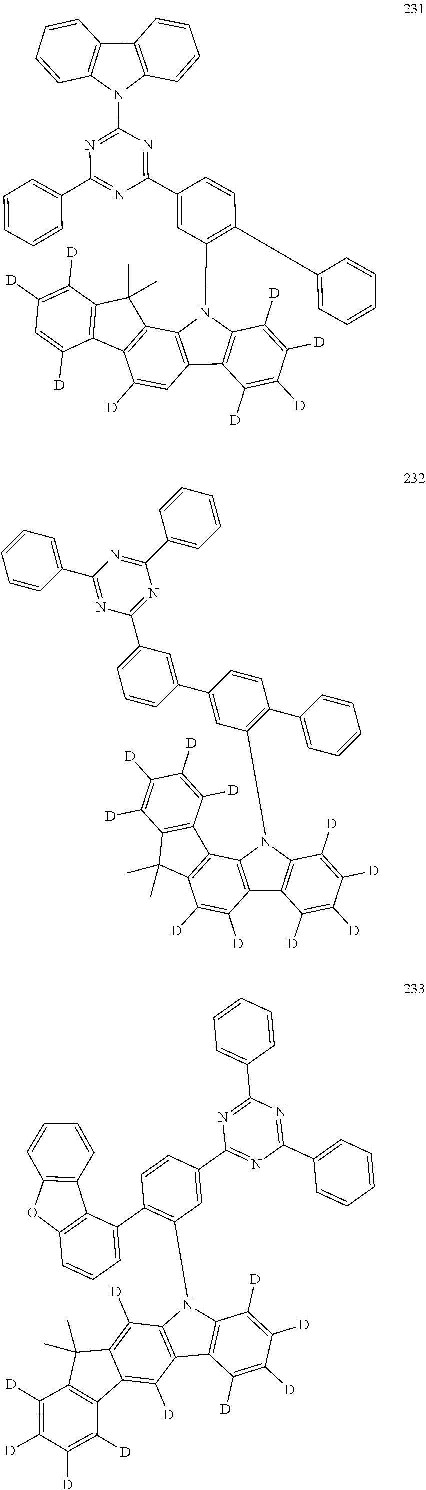 [SMILES: [2H]c1cc([2H])c2c(c1[2H])C(C)(C)c1c-2c([2H])cc2c3c([2H])c([2H])c([2H])c([2H])c3n(-c3cc(-c4nc(-c5ccccc5)nc(-n5c6ccccc6c6ccccc65)n4)ccc3-c3ccccc3)c12 ; [2H]c1c([2H])c([2H])c2c(c1[2H])-c1c(c([2H])c([2H])c3c4c([2H])c([2H])c([2H])c([2H])c4n(-c4cc(-c5cccc(-c6nc(-c7ccccc7)nc(-c7ccccc7)n6)c5)ccc4-c4ccccc4)c13)C2(C)C ; [2H]c1c([2H])c([2H])c2c(c1[2H])-c1c(c([2H])c3c(c1[2H])c1c([2H])c([2H])c([2H])c([2H])c1n3-c1cc(-c3nc(-c4ccccc4)nc(-c4ccccc4)n3)ccc1-c1cccc3oc4ccccc4c13)C2(C)C]

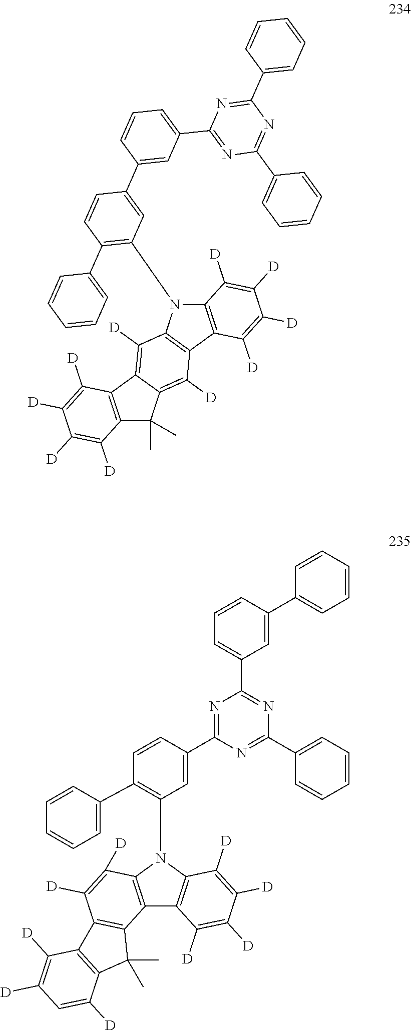 [SMILES: [2H]c1c([2H])c([2H])c2c(c1[2H])-c1c(c([2H])c3c4c([2H])c([2H])c([2H])c([2H])c4n(-c4cc(-c5cccc(-c6nc(-c7ccccc7)nc(-c7ccccc7)n6)c5)ccc4-c4ccccc4)c3c1[2H])C2(C)C ; [2H]c1cc([2H])c2c(c1[2H])-c1c([2H])c([2H])c3c(c1C2(C)C)c1c([2H])c([2H])c([2H])c([2H])c1n3-c1cc(-c2nc(-c3ccccc3)nc(-c3cccc(-c4ccccc4)c3)n2)ccc1-c1ccccc1]

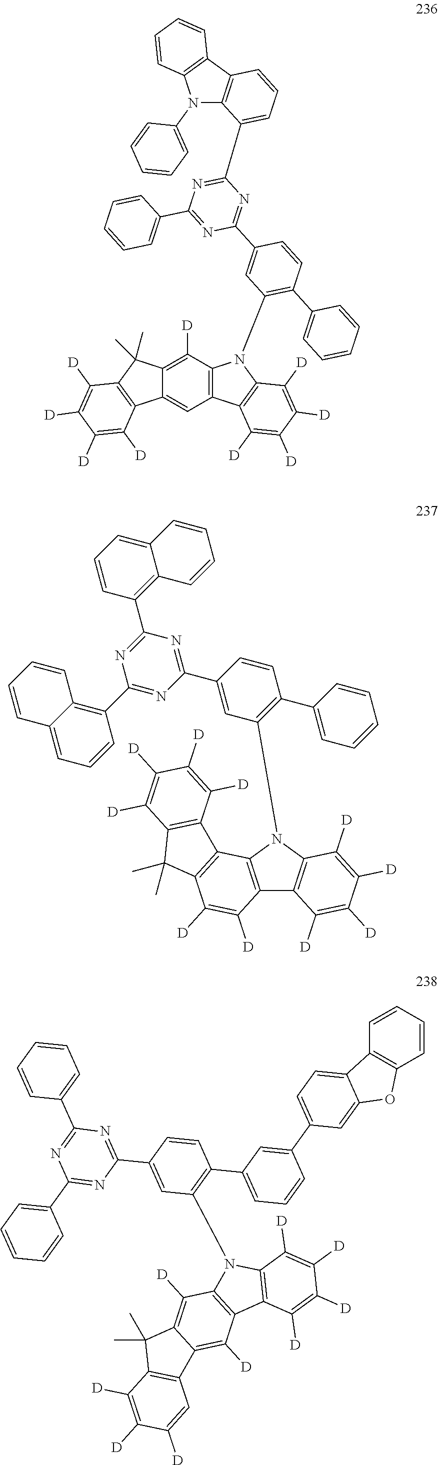 [SMILES: [2H]c1c([2H])c([2H])c2c(c1[2H])-c1cc3c4c([2H])c([2H])c([2H])c([2H])c4n(-c4cc(-c5nc(-c6ccccc6)nc(-c6cccc7c8ccccc8n(-c8ccccc8)c67)n5)ccc4-c4ccccc4)c3c([2H])c1C2(C)C ; [2H]c1c([2H])c([2H])c2c(c1[2H])-c1c(c([2H])c([2H])c3c4c([2H])c([2H])c([2H])c([2H])c4n(-c4cc(-c5nc(-c6cccc7ccccc67)nc(-c6cccc7ccccc67)n5)ccc4-c4ccccc4)c13)C2(C)C ; [2H]c1cc2c(c([2H])c1[2H])C(C)(C)c1c-2c([2H])c2c3c([2H])c([2H])c([2H])c([2H])c3n(-c3cc(-c4nc(-c5ccccc5)nc(-c5ccccc5)n4)ccc3-c3cccc(-c4ccc5c(c4)oc4ccccc45)c3)c2c1[2H]]

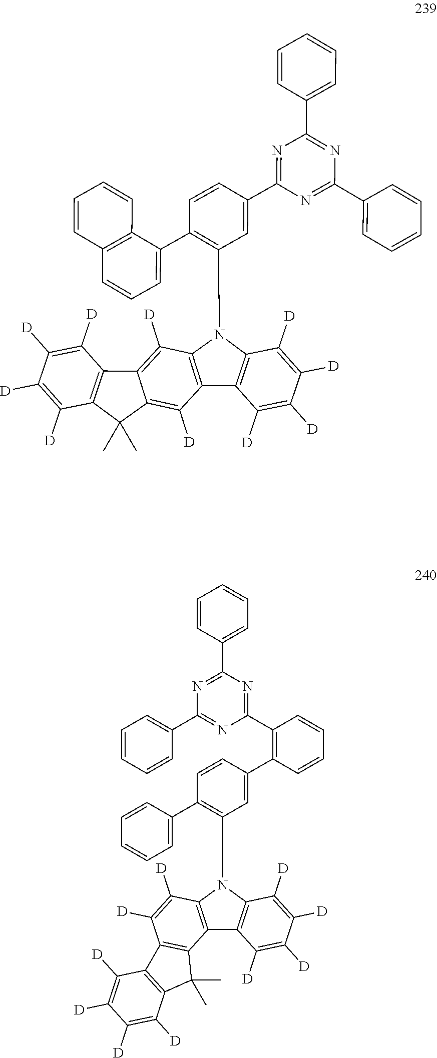 [SMILES: [2H]c1c([2H])c([2H])c2c(c1[2H])-c1c(c([2H])c3c4c([2H])c([2H])c([2H])c([2H])c4n(-c4cc(-c5nc(-c6ccccc6)nc(-c6ccccc6)n5)ccc4-c4cccc5ccccc45)c3c1[2H])C2(C)C ; [2H]c1c([2H])c([2H])c2c(c1[2H])-c1c([2H])c([2H])c3c(c1C2(C)C)c1c([2H])c([2H])c([2H])c([2H])c1n3-c1cc(-c2ccccc2-c2nc(-c3ccccc3)nc(-c3ccccc3)n2)ccc1-c1ccccc1]

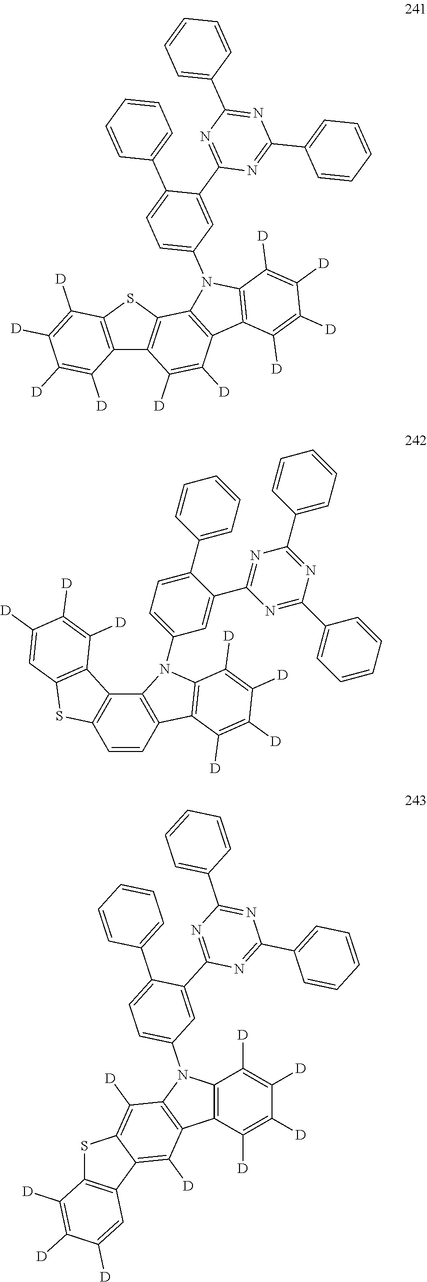 [SMILES: [2H]c1c([2H])c([2H])c2c(sc3c2c([2H])c([2H])c2c4c([2H])c([2H])c([2H])c([2H])c4n(-c4ccc(-c5ccccc5)c(-c5nc(-c6ccccc6)nc(-c6ccccc6)n5)c4)c32)c1[2H] ; [2H]c1cc2sc3ccc4c5c([2H])c([2H])c([2H])c([2H])c5n(-c5ccc(-c6ccccc6)c(-c6nc(-c7ccccc7)nc(-c7ccccc7)n6)c5)c4c3c2c([2H])c1[2H] ; [2H]c1cc2c(sc3c([2H])c4c(c([2H])c32)c2c([2H])c([2H])c([2H])c([2H])c2n4-c2ccc(-c3ccccc3)c(-c3nc(-c4ccccc4)nc(-c4ccccc4)n3)c2)c([2H])c1[2H]]

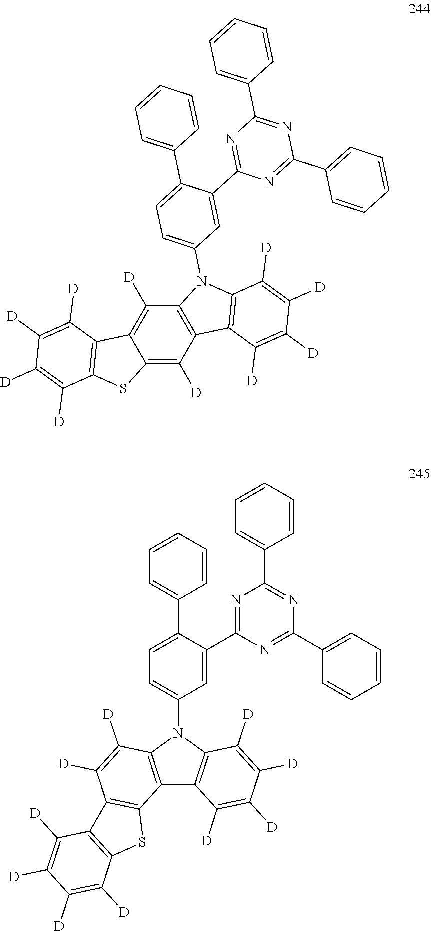 [SMILES: [2H]c1c([2H])c([2H])c2c(sc3c([2H])c4c5c([2H])c([2H])c([2H])c([2H])c5n(-c5ccc(-c6ccccc6)c(-c6nc(-c7ccccc7)nc(-c7ccccc7)n6)c5)c4c([2H])c32)c1[2H] ; [2H]c1c([2H])c([2H])c2c(sc3c2c([2H])c([2H])c2c3c3c([2H])c([2H])c([2H])c([2H])c3n2-c2ccc(-c3ccccc3)c(-c3nc(-c4ccccc4)nc(-c4ccccc4)n3)c2)c1[2H]]

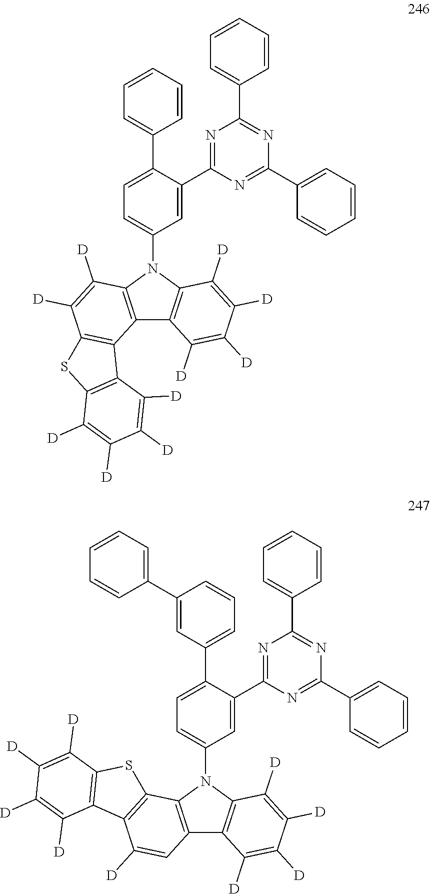 [SMILES: [2H]c1c([2H])c([2H])c2c(sc3c([2H])c([2H])c4c(c5c([2H])c([2H])c([2H])c([2H])c5n4-c4ccc(-c5ccccc5)c(-c5nc(-c6ccccc6)nc(-c6ccccc6)n5)c4)c32)c1[2H] ; [2H]c1c([2H])c([2H])c2c(sc3c2c([2H])cc2c4c([2H])c([2H])c([2H])c([2H])c4n(-c4ccc(-c5cccc(-c6ccccc6)c5)c(-c5nc(-c6ccccc6)nc(-c6ccccc6)n5)c4)c23)c1[2H]]

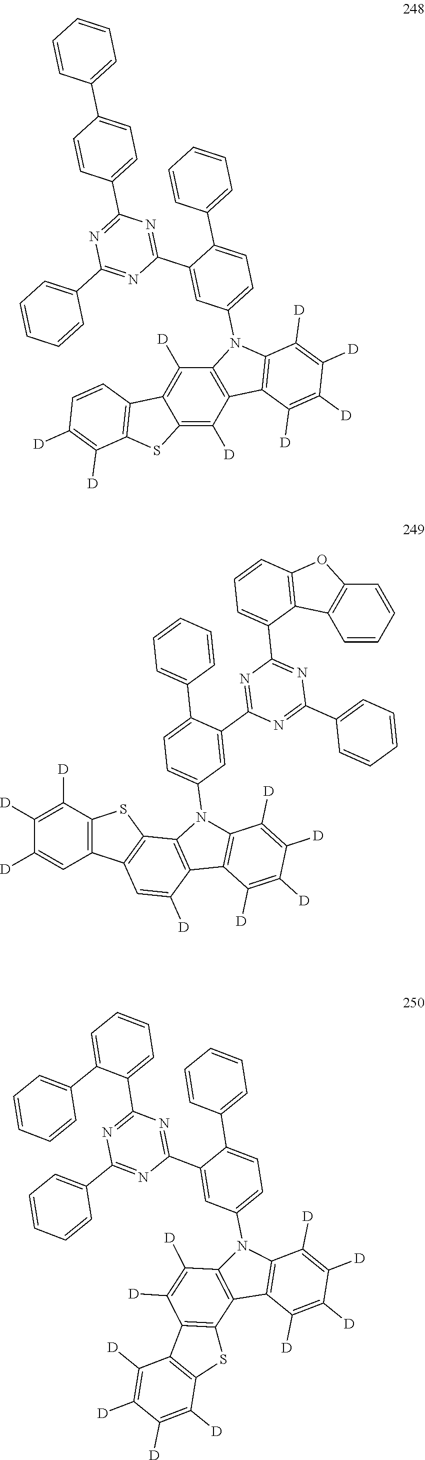 [SMILES: [2H]c1ccc2c(sc3c([2H])c4c5c([2H])c([2H])c([2H])c([2H])c5n(-c5ccc(-c6ccccc6)c(-c6nc(-c7ccccc7)nc(-c7ccc(-c8ccccc8)cc7)n6)c5)c4c([2H])c32)c1[2H] ; [2H]c1cc2c(sc3c2cc([2H])c2c4c([2H])c([2H])c([2H])c([2H])c4n(-c4ccc(-c5ccccc5)c(-c5nc(-c6ccccc6)nc(-c6cccc7oc8ccccc8c67)n5)c4)c32)c([2H])c1[2H] ; [2H]c1c([2H])c([2H])c2c(sc3c2c([2H])c([2H])c2c3c3c([2H])c([2H])c([2H])c([2H])c3n2-c2ccc(-c3ccccc3)c(-c3nc(-c4ccccc4)nc(-c4ccccc4-c4ccccc4)n3)c2)c1[2H]]

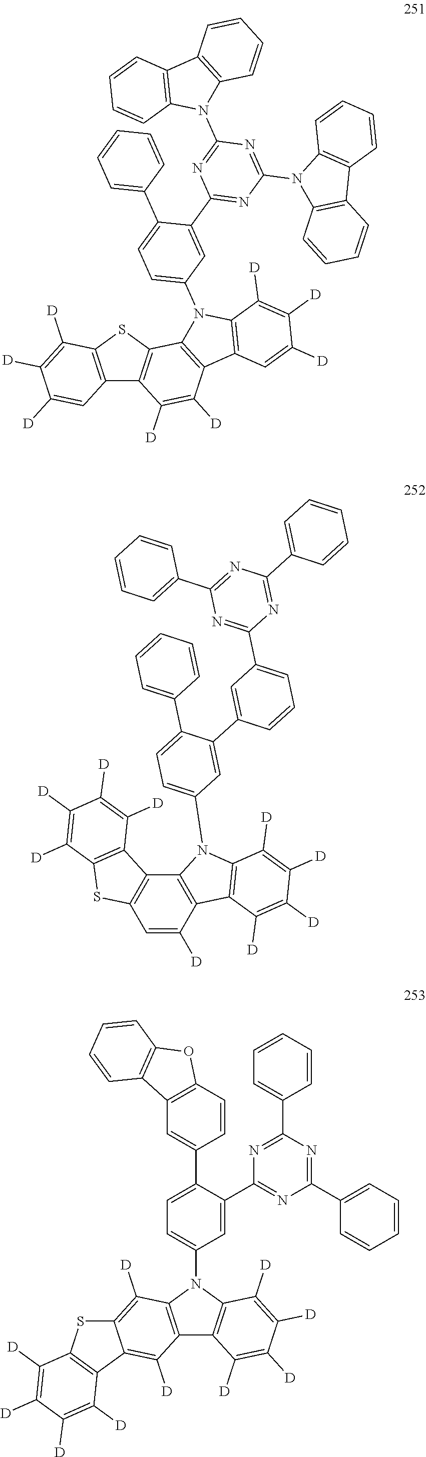 [SMILES: [2H]c1cc2c(sc3c2c([2H])c([2H])c2c4cc([2H])c([2H])c([2H])c4n(-c4ccc(-c5ccccc5)c(-c5nc(-n6c7ccccc7c7ccccc76)nc(-n6c7ccccc7c7ccccc76)n5)c4)c32)c([2H])c1[2H] ; [2H]c1c([2H])c([2H])c2c(sc3cc([2H])c4c5c([2H])c([2H])c([2H])c([2H])c5n(-c5ccc(-c6ccccc6)c(-c6cccc(-c7nc(-c8ccccc8)nc(-c8ccccc8)n7)c6)c5)c4c32)c1[2H] ; [2H]c1c([2H])c([2H])c2c(sc3c([2H])c4c(c([2H])c32)c2c([2H])c([2H])c([2H])c([2H])c2n4-c2ccc(-c3ccc4oc5ccccc5c4c3)c(-c3nc(-c4ccccc4)nc(-c4ccccc4)n3)c2)c1[2H]]

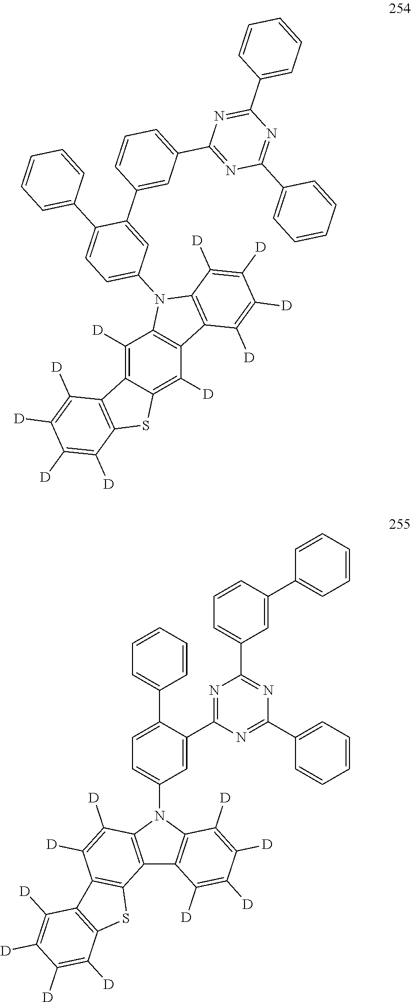 [SMILES: [2H]c1c([2H])c([2H])c2c(sc3c([2H])c4c5c([2H])c([2H])c([2H])c([2H])c5n(-c5ccc(-c6ccccc6)c(-c6cccc(-c7nc(-c8ccccc8)nc(-c8ccccc8)n7)c6)c5)c4c([2H])c32)c1[2H] ; [2H]c1c([2H])c([2H])c2c(sc3c2c([2H])c([2H])c2c3c3c([2H])c([2H])c([2H])c([2H])c3n2-c2ccc(-c3ccccc3)c(-c3nc(-c4ccccc4)nc(-c4cccc(-c5ccccc5)c4)n3)c2)c1[2H]]

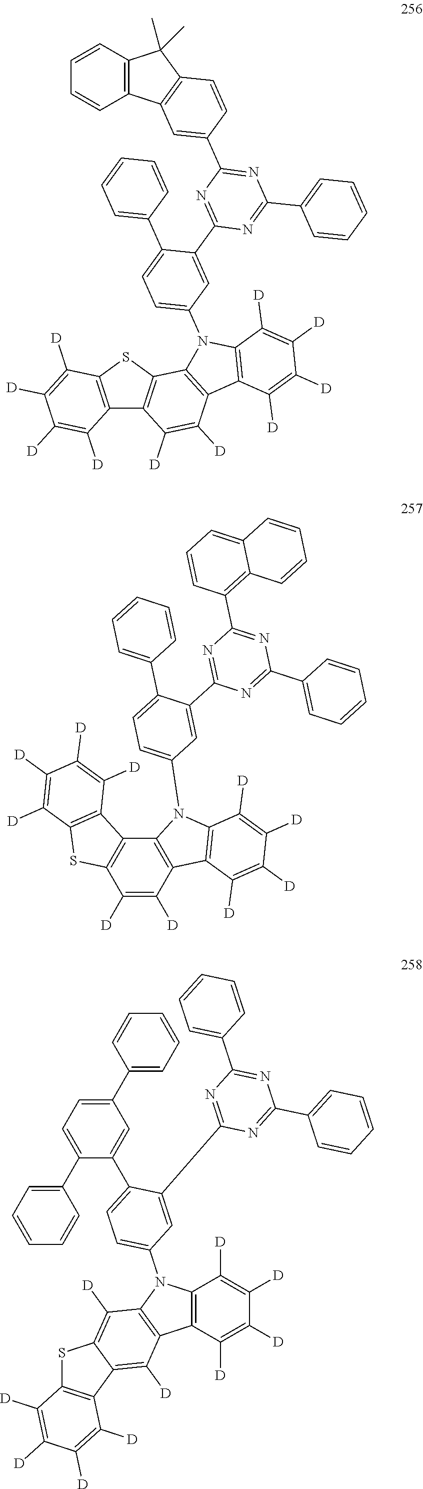 [SMILES: [2H]c1c([2H])c([2H])c2c(sc3c2c([2H])c([2H])c2c4c([2H])c([2H])c([2H])c([2H])c4n(-c4ccc(-c5ccccc5)c(-c5nc(-c6ccccc6)nc(-c6ccc7c(c6)-c6ccccc6C7(C)C)n5)c4)c32)c1[2H] ; [2H]c1c([2H])c([2H])c2c(sc3c([2H])c([2H])c4c5c([2H])c([2H])c([2H])c([2H])c5n(-c5ccc(-c6ccccc6)c(-c6nc(-c7ccccc7)nc(-c7cccc8ccccc78)n6)c5)c4c32)c1[2H] ; [2H]c1c([2H])c([2H])c2c(sc3c([2H])c4c(c([2H])c32)c2c([2H])c([2H])c([2H])c([2H])c2n4-c2ccc(-c3cc(-c4ccccc4)ccc3-c3ccccc3)c(-c3nc(-c4ccccc4)nc(-c4ccccc4)n3)c2)c1[2H]]

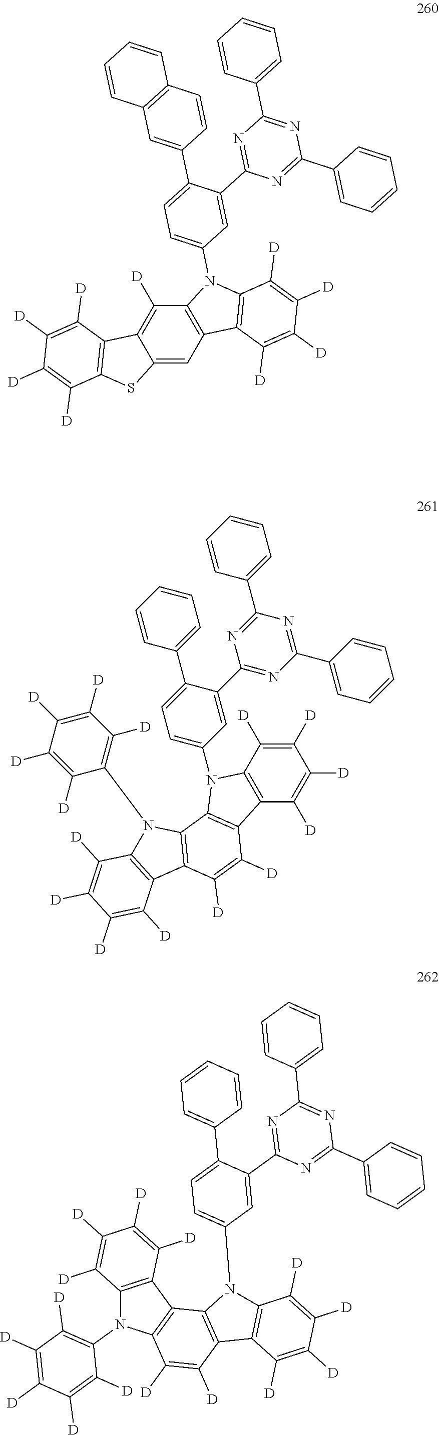

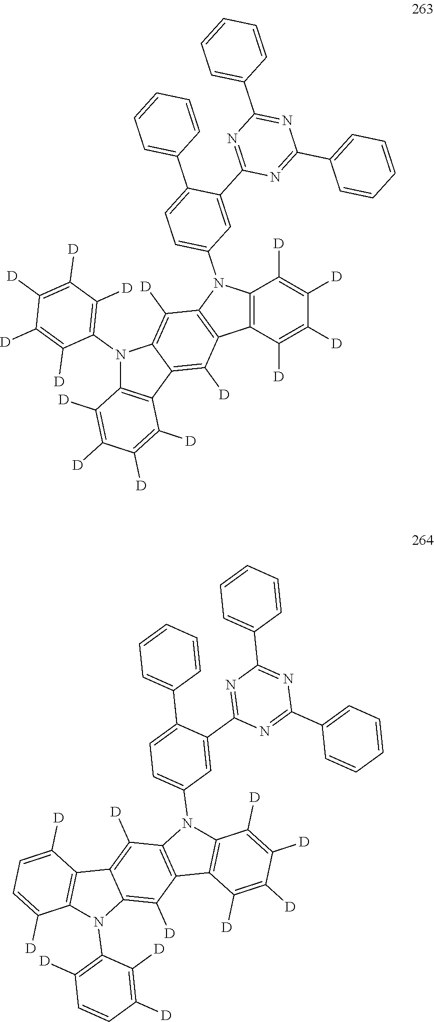 [SMILES: [2H]c1c([2H])c([2H])c(-n2c3c([2H])c([2H])c([2H])c([2H])c3c3c([2H])c4c5c([2H])c([2H])c([2H])c([2H])c5n(-c5ccc(-c6ccccc6)c(-c6nc(-c7ccccc7)nc(-c7ccccc7)n6)c5)c4c([2H])c32)c([2H])c1[2H] ; [2H]c1ccc([2H])c(-n2c3c([2H])ccc([2H])c3c3c([2H])c4c(c([2H])c32)c2c([2H])c([2H])c([2H])c([2H])c2n4-c2ccc(-c3ccccc3)c(-c3nc(-c4ccccc4)nc(-c4ccccc4)n3)c2)c1[2H]]

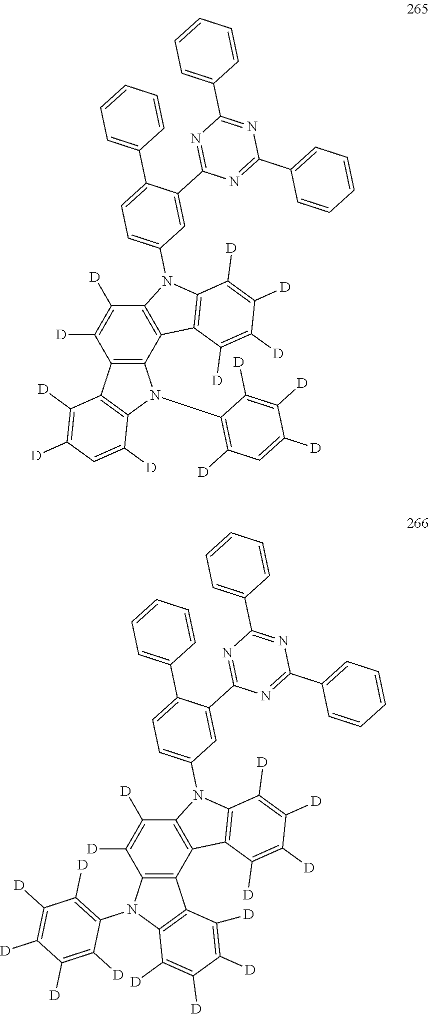 [SMILES: [2H]c1cc([2H])c(-n2c3c([2H])cc([2H])c([2H])c3c3c([2H])c([2H])c4c(c5c([2H])c([2H])c([2H])c([2H])c5n4-c4ccc(-c5ccccc5)c(-c5nc(-c6ccccc6)nc(-c6ccccc6)n5)c4)c32)c([2H])c1[2H] ; [2H]c1c([2H])c([2H])c(-n2c3c([2H])c([2H])c([2H])c([2H])c3c3c4c5c([2H])c([2H])c([2H])c([2H])c5n(-c5ccc(-c6ccccc6)c(-c6nc(-c7ccccc7)nc(-c7ccccc7)n6)c5)c4c([2H])c([2H])c32)c([2H])c1[2H]]

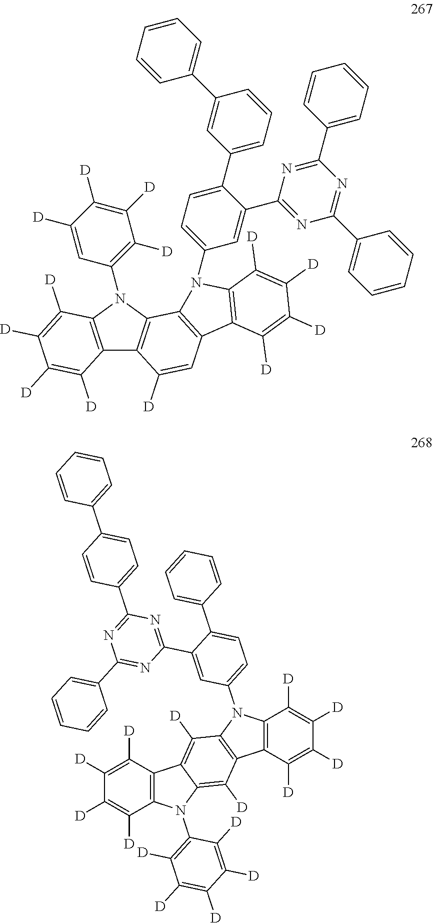 [SMILES: [2H]c1cc(-n2c3c([2H])c([2H])c([2H])c([2H])c3c3c([2H])cc4c5c([2H])c([2H])c([2H])c([2H])c5n(-c5ccc(-c6cccc(-c7ccccc7)c6)c(-c6nc(-c7ccccc7)nc(-c7ccccc7)n6)c5)c4c32)c([2H])c([2H])c1[2H] ; [2H]c1c([2H])c([2H])c(-n2c3c([2H])c([2H])c([2H])c([2H])c3c3c([2H])c4c(c([2H])c32)c2c([2H])c([2H])c([2H])c([2H])c2n4-c2ccc(-c3ccccc3)c(-c3nc(-c4ccccc4)nc(-c4ccc(-c5ccccc5)cc4)n3)c2)c([2H])c1[2H]]

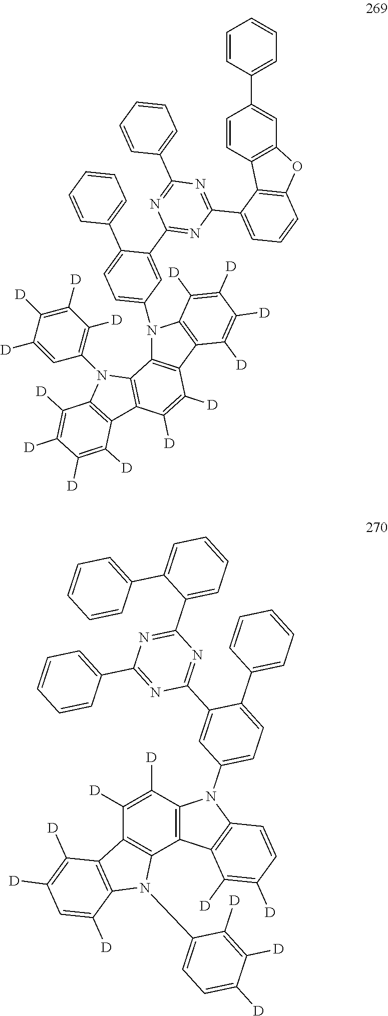 [SMILES: [2H]c1cc(-n2c3c([2H])c([2H])c([2H])c([2H])c3c3c([2H])c([2H])c4c5c([2H])c([2H])c([2H])c([2H])c5n(-c5ccc(-c6ccccc6)c(-c6nc(-c7ccccc7)nc(-c7cccc8oc9cc(-c%10ccccc%10)ccc9c78)n6)c5)c4c32)c([2H])c([2H])c1[2H] ; [2H]c1ccc(-n2c3c([2H])cc([2H])c([2H])c3c3c([2H])c([2H])c4c(c5c([2H])c([2H])ccc5n4-c4ccc(-c5ccccc5)c(-c5nc(-c6ccccc6)nc(-c6ccccc6-c6ccccc6)n5)c4)c32)c([2H])c1[2H]]

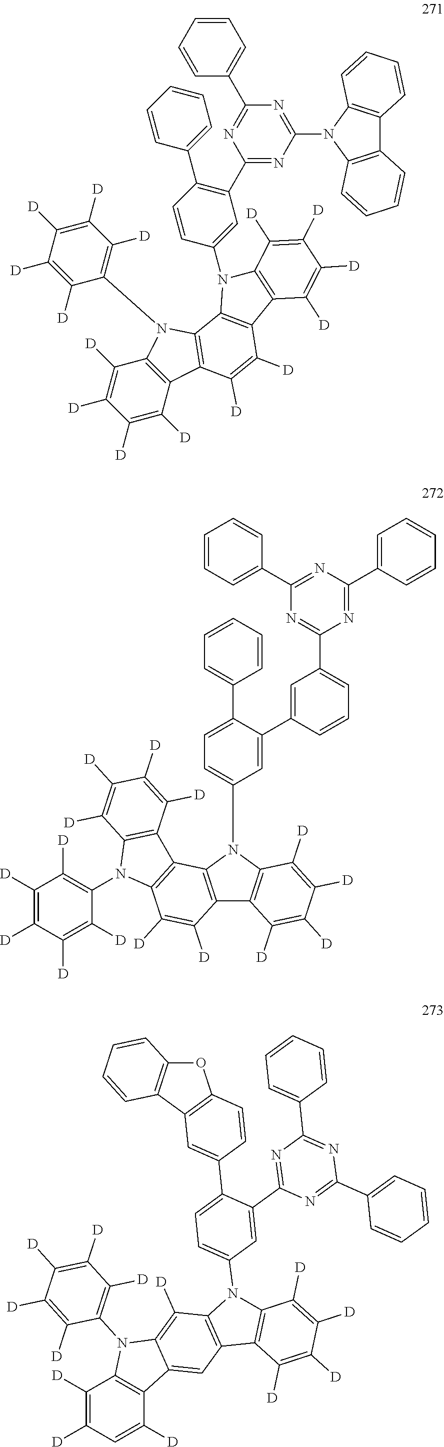

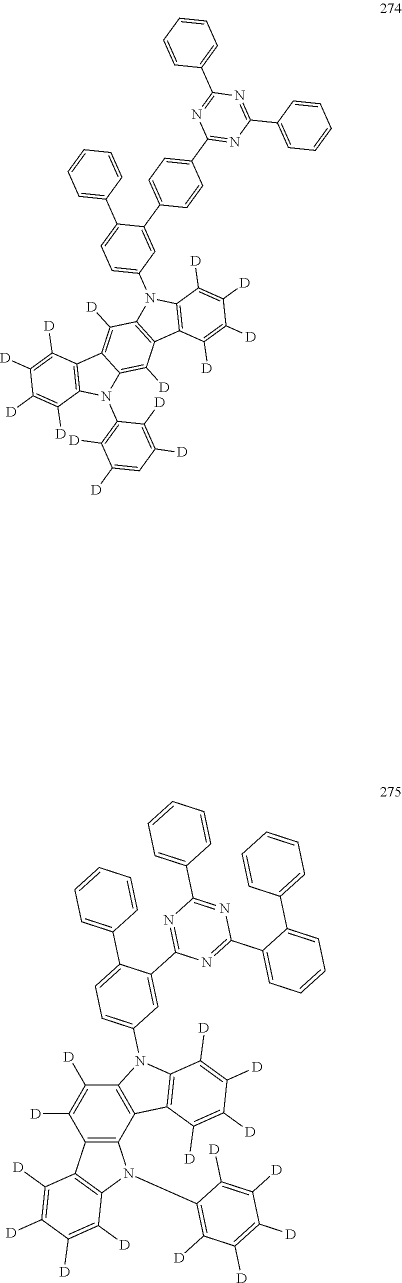 [SMILES: [2H]c1cc([2H])c([2H])c(-n2c3c([2H])c([2H])c([2H])c([2H])c3c3c([2H])c4c(c([2H])c32)c2c([2H])c([2H])c([2H])c([2H])c2n4-c2ccc(-c3ccccc3)c(-c3ccc(-c4nc(-c5ccccc5)nc(-c5ccccc5)n4)cc3)c2)c1[2H] ; [2H]c1c([2H])c([2H])c(-n2c3c([2H])c([2H])c([2H])c([2H])c3c3c([2H])c([2H])c4c(c5c([2H])c([2H])c([2H])c([2H])c5n4-c4ccc(-c5ccccc5)c(-c5nc(-c6ccccc6)nc(-c6ccccc6-c6ccccc6)n5)c4)c32)c([2H])c1[2H]]

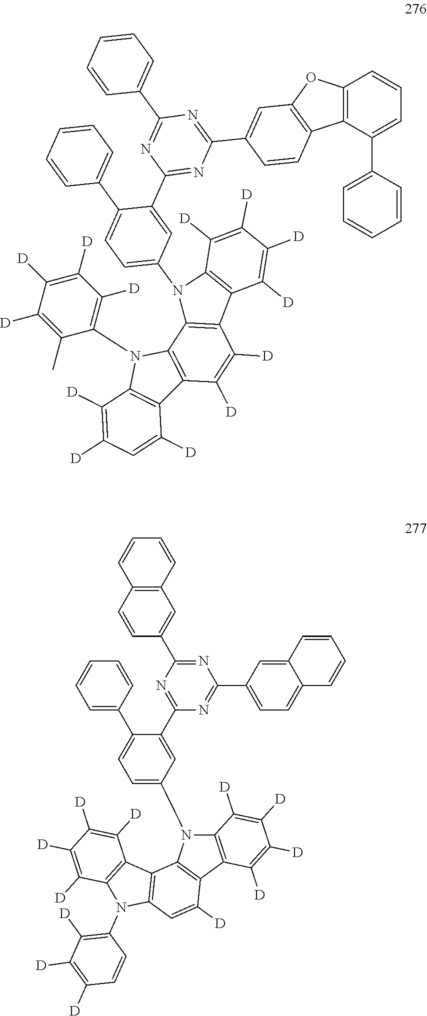 [SMILES: [2H]c1cc([2H])c2c3c([2H])c([2H])c4c5c([2H])c([2H])c([2H])c([2H])c5n(-c5ccc(-c6ccccc6)c(-c6nc(-c7ccccc7)nc(-c7ccc8c(c7)oc7cccc(-c9ccccc9)c78)n6)c5)c4c3n(-c3c([2H])c([2H])c([2H])c([2H])c3C)c2c1[2H] ; [2H]c1ccc(-n2c3cc([2H])c4c5c([2H])c([2H])c([2H])c([2H])c5n(-c5ccc(-c6ccccc6)c(-c6nc(-c7ccc8ccccc8c7)nc(-c7ccc8ccccc8c7)n6)c5)c4c3c3c([2H])c([2H])c([2H])c([2H])c32)c([2H])c1[2H]]

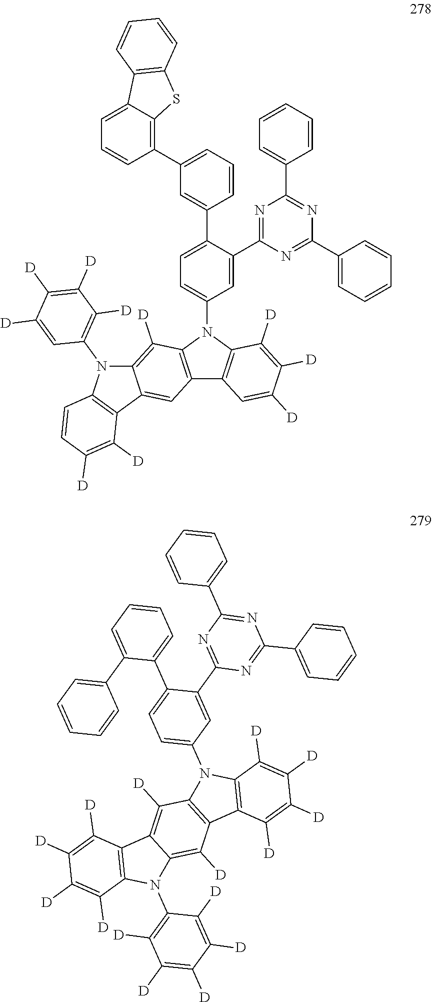 [SMILES: [2H]c1cc(-n2c3ccc([2H])c([2H])c3c3cc4c5cc([2H])c([2H])c([2H])c5n(-c5ccc(-c6cccc(-c7cccc8c7sc7ccccc78)c6)c(-c6nc(-c7ccccc7)nc(-c7ccccc7)n6)c5)c4c([2H])c32)c([2H])c([2H])c1[2H] ; [2H]c1c([2H])c([2H])c(-n2c3c([2H])c([2H])c([2H])c([2H])c3c3c([2H])c4c(c([2H])c32)c2c([2H])c([2H])c([2H])c([2H])c2n4-c2ccc(-c3ccccc3-c3ccccc3)c(-c3nc(-c4ccccc4)nc(-c4ccccc4)n3)c2)c([2H])c1[2H]]

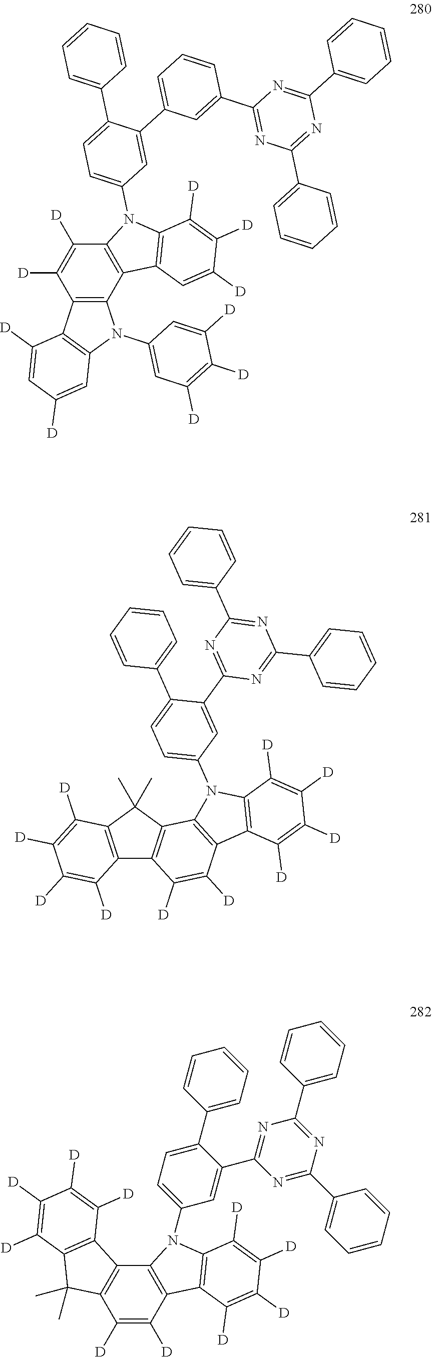 [SMILES: [2H]c1cc([2H])c2c3c([2H])c([2H])c4c(c5cc([2H])c([2H])c([2H])c5n4-c4ccc(-c5ccccc5)c(-c5cccc(-c6nc(-c7ccccc7)nc(-c7ccccc7)n6)c5)c4)c3n(-c3cc([2H])c([2H])c([2H])c3)c2c1 ; [2H]c1c([2H])c([2H])c2c(c1[2H])-c1c([2H])c([2H])c3c4c([2H])c([2H])c([2H])c([2H])c4n(-c4ccc(-c5ccccc5)c(-c5nc(-c6ccccc6)nc(-c6ccccc6)n5)c4)c3c1C2(C)C ; [2H]c1c([2H])c([2H])c2c(c1[2H])-c1c(c([2H])c([2H])c3c4c([2H])c([2H])c([2H])c([2H])c4n(-c4ccc(-c5ccccc5)c(-c5nc(-c6ccccc6)nc(-c6ccccc6)n5)c4)c13)C2(C)C]

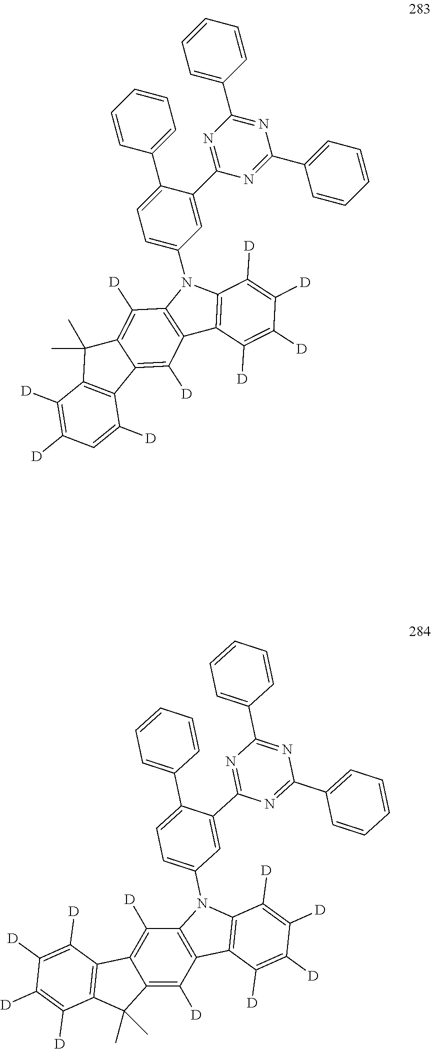 [SMILES: [2H]c1cc([2H])c2c(c1[2H])C(C)(C)c1c-2c([2H])c2c3c([2H])c([2H])c([2H])c([2H])c3n(-c3ccc(-c4ccccc4)c(-c4nc(-c5ccccc5)nc(-c5ccccc5)n4)c3)c2c1[2H] ; [2H]c1c([2H])c([2H])c2c(c1[2H])-c1c(c([2H])c3c4c([2H])c([2H])c([2H])c([2H])c4n(-c4ccc(-c5ccccc5)c(-c5nc(-c6ccccc6)nc(-c6ccccc6)n5)c4)c3c1[2H])C2(C)C]

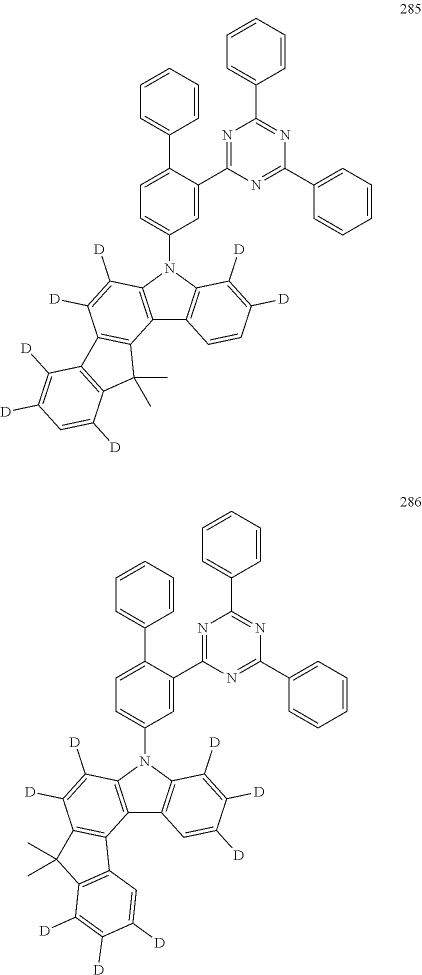 [SMILES: [2H]c1cc([2H])c2c(c1[2H])-c1c([2H])c([2H])c3c(c1C2(C)C)c1ccc([2H])c([2H])c1n3-c1ccc(-c2ccccc2)c(-c2nc(-c3ccccc3)nc(-c3ccccc3)n2)c1 ; [2H]c1cc2c(c([2H])c1[2H])C(C)(C)c1c([2H])c([2H])c3c(c1-2)c1cc([2H])c([2H])c([2H])c1n3-c1ccc(-c2ccccc2)c(-c2nc(-c3ccccc3)nc(-c3ccccc3)n2)c1]

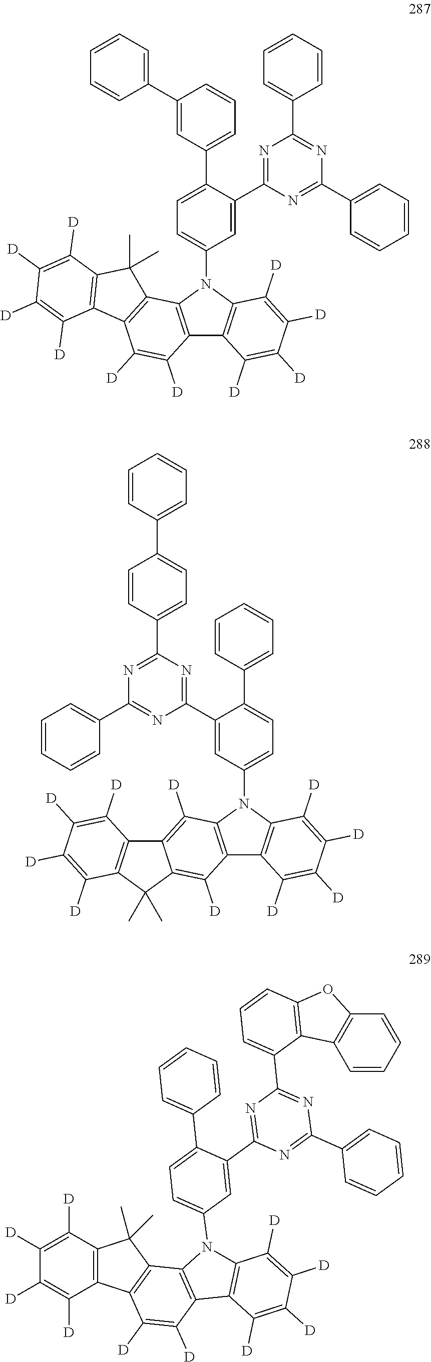 [SMILES: [2H]c1c([2H])c([2H])c2c(c1[2H])-c1c([2H])c([2H])c3c4c([2H])c([2H])c([2H])c([2H])c4n(-c4ccc(-c5cccc(-c6ccccc6)c5)c(-c5nc(-c6ccccc6)nc(-c6ccccc6)n5)c4)c3c1C2(C)C ; [2H]c1c([2H])c([2H])c2c(c1[2H])-c1c(c([2H])c3c4c([2H])c([2H])c([2H])c([2H])c4n(-c4ccc(-c5ccccc5)c(-c5nc(-c6ccccc6)nc(-c6ccc(-c7ccccc7)cc6)n5)c4)c3c1[2H])C2(C)C ; [2H]c1c([2H])c([2H])c2c(c1[2H])-c1c([2H])c([2H])c3c4c([2H])c([2H])c([2H])c([2H])c4n(-c4ccc(-c5ccccc5)c(-c5nc(-c6ccccc6)nc(-c6cccc7oc8ccccc8c67)n5)c4)c3c1C2(C)C]

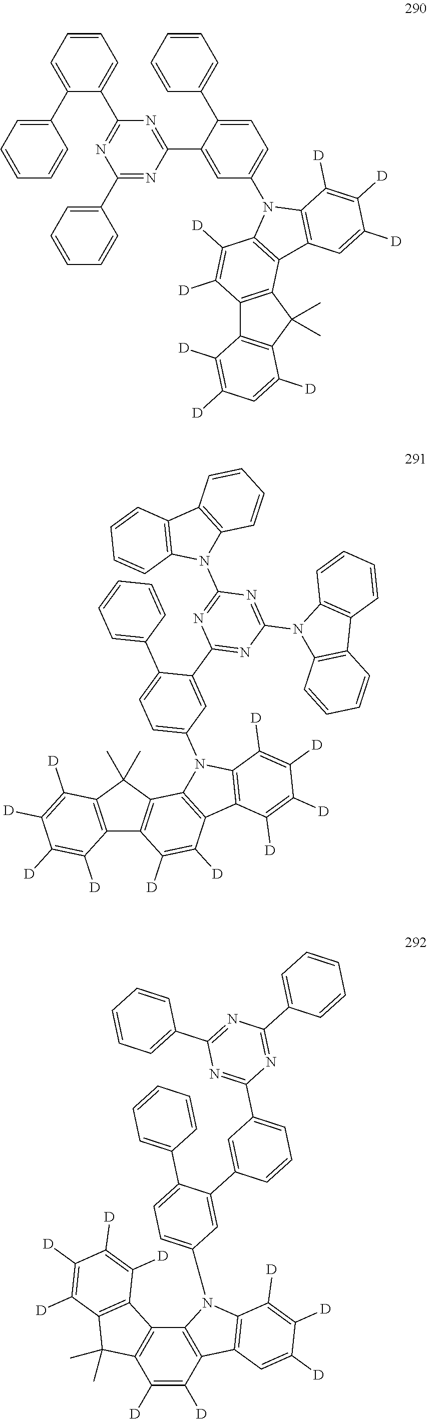 [SMILES: [2H]c1cc([2H])c2c(c1[2H])-c1c([2H])c([2H])c3c(c1C2(C)C)c1cc([2H])c([2H])c([2H])c1n3-c1ccc(-c2ccccc2)c(-c2nc(-c3ccccc3)nc(-c3ccccc3-c3ccccc3)n2)c1 ; [2H]c1c([2H])c([2H])c2c(c1[2H])-c1c([2H])c([2H])c3c4c([2H])c([2H])c([2H])c([2H])c4n(-c4ccc(-c5ccccc5)c(-c5nc(-n6c7ccccc7c7ccccc76)nc(-n6c7ccccc7c7ccccc76)n5)c4)c3c1C2(C)C ; [2H]c1cc2c3c([2H])c([2H])c4c(c3n(-c3ccc(-c5ccccc5)c(-c5cccc(-c6nc(-c7ccccc7)nc(-c7ccccc7)n6)c5)c3)c2c([2H])c1[2H])-c1c([2H])c([2H])c([2H])c([2H])c1C4(C)C]

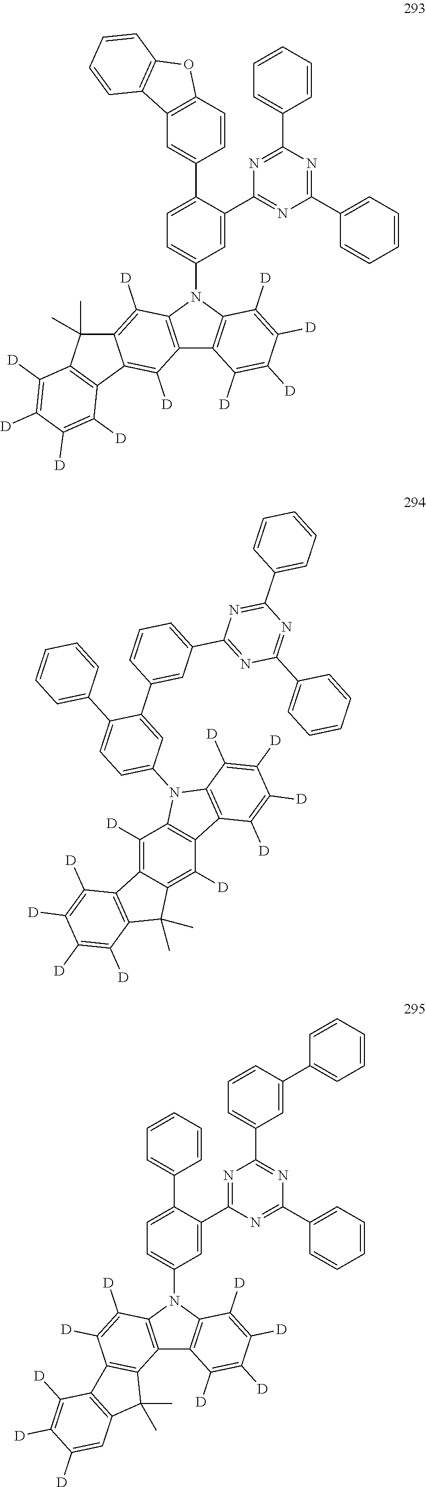 [SMILES: [2H]c1c([2H])c([2H])c2c(c1[2H])-c1c(c([2H])c3c(c1[2H])c1c([2H])c([2H])c([2H])c([2H])c1n3-c1ccc(-c3ccc4oc5ccccc5c4c3)c(-c3nc(-c4ccccc4)nc(-c4ccccc4)n3)c1)C2(C)C ; [2H]c1c([2H])c([2H])c2c(c1[2H])-c1c(c([2H])c3c4c([2H])c([2H])c([2H])c([2H])c4n(-c4ccc(-c5ccccc5)c(-c5cccc(-c6nc(-c7ccccc7)nc(-c7ccccc7)n6)c5)c4)c3c1[2H])C2(C)C ; [2H]c1cc2c(c([2H])c1[2H])-c1c([2H])c([2H])c3c(c1C2(C)C)c1c([2H])c([2H])c([2H])c([2H])c1n3-c1ccc(-c2ccccc2)c(-c2nc(-c3ccccc3)nc(-c3cccc(-c4ccccc4)c3)n2)c1]

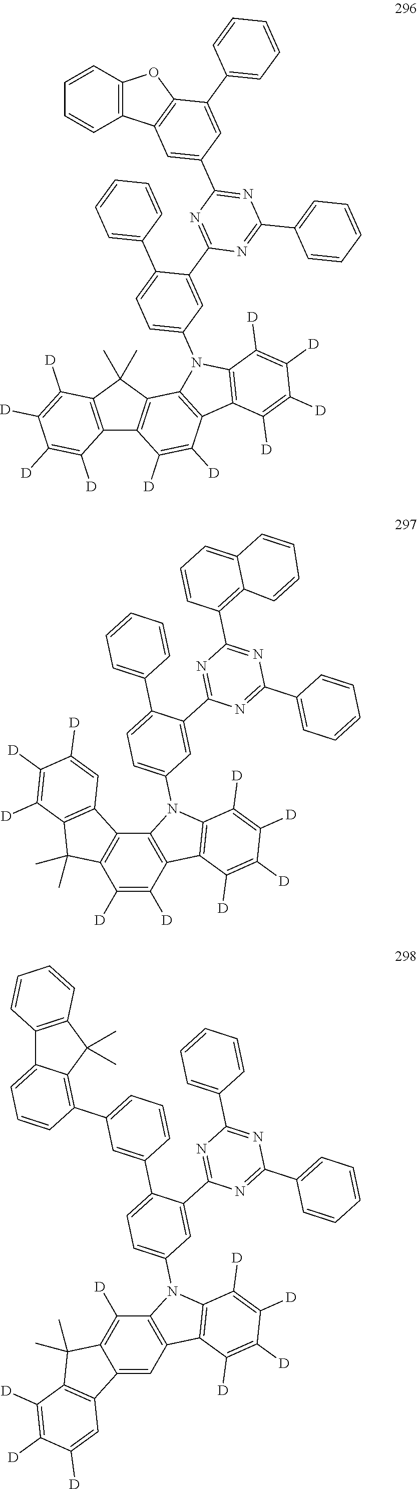 [SMILES: [2H]c1c([2H])c([2H])c2c(c1[2H])-c1c([2H])c([2H])c3c4c([2H])c([2H])c([2H])c([2H])c4n(-c4ccc(-c5ccccc5)c(-c5nc(-c6ccccc6)nc(-c6cc(-c7ccccc7)c7oc8ccccc8c7c6)n5)c4)c3c1C2(C)C ; [2H]c1cc2c(c([2H])c1[2H])C(C)(C)c1c([2H])c([2H])c3c4c([2H])c([2H])c([2H])c([2H])c4n(-c4ccc(-c5ccccc5)c(-c5nc(-c6ccccc6)nc(-c6cccc7ccccc67)n5)c4)c3c1-2 ; [2H]c1cc2c(c([2H])c1[2H])C(C)(C)c1c-2cc2c3c([2H])c([2H])c([2H])c([2H])c3n(-c3ccc(-c4cccc(-c5cccc6c5C(C)(C)c5ccccc5-6)c4)c(-c4nc(-c5ccccc5)nc(-c5ccccc5)n4)c3)c2c1[2H]]

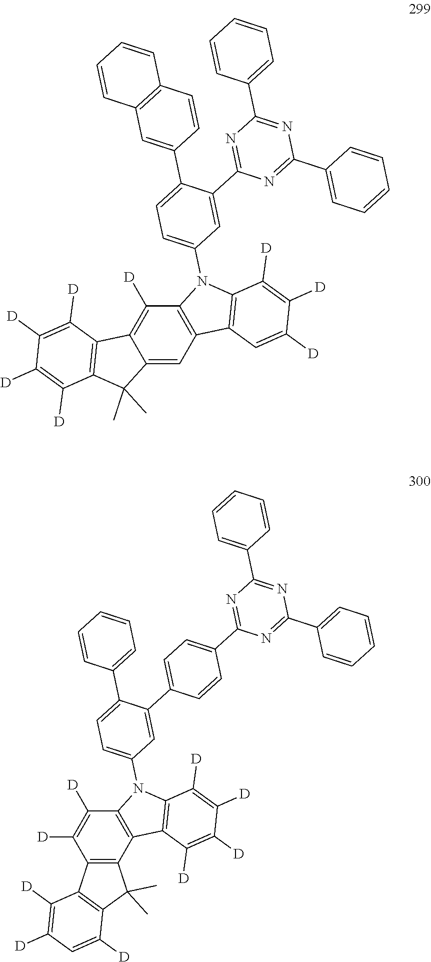 [SMILES: [2H]c1cc2c3cc4c(c([2H])c3n(-c3ccc(-c5ccc6ccccc6c5)c(-c5nc(-c6ccccc6)nc(-c6ccccc6)n5)c3)c2c([2H])c1[2H])-c1c([2H])c([2H])c([2H])c([2H])c1C4(C)C ; [2H]c1cc([2H])c2c(c1[2H])-c1c([2H])c([2H])c3c(c1C2(C)C)c1c([2H])c([2H])c([2H])c([2H])c1n3-c1ccc(-c2ccccc2)c(-c2ccc(-c3nc(-c4ccccc4)nc(-c4ccccc4)n3)cc2)c1]

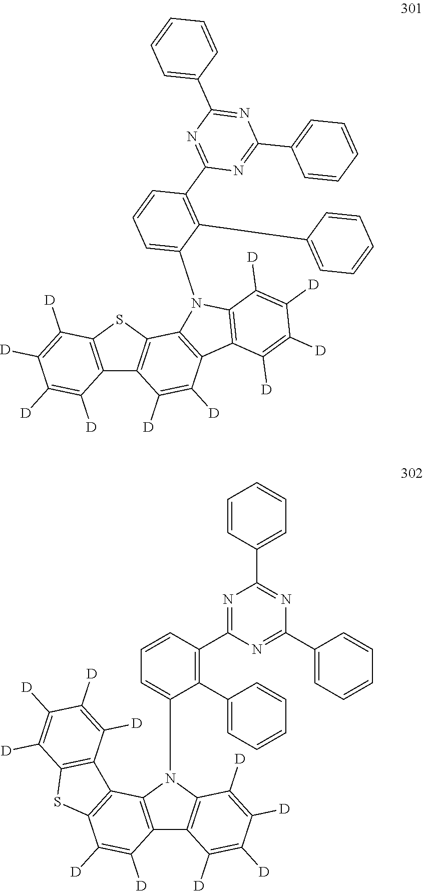 [SMILES: [2H]c1c([2H])c([2H])c2c(sc3c2c([2H])c([2H])c2c4c([2H])c([2H])c([2H])c([2H])c4n(-c4cccc(-c5nc(-c6ccccc6)nc(-c6ccccc6)n5)c4-c4ccccc4)c32)c1[2H] ; [2H]c1c([2H])c([2H])c2c(sc3c([2H])c([2H])c4c5c([2H])c([2H])c([2H])c([2H])c5n(-c5cccc(-c6nc(-c7ccccc7)nc(-c7ccccc7)n6)c5-c5ccccc5)c4c32)c1[2H]]

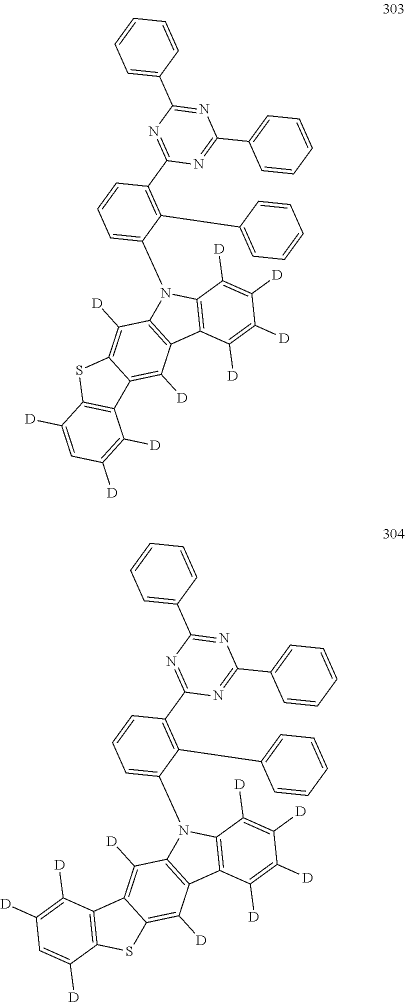 [SMILES: [2H]c1cc([2H])c2sc3c([2H])c4c(c([2H])c3c2c1[2H])c1c([2H])c([2H])c([2H])c([2H])c1n4-c1cccc(-c2nc(-c3ccccc3)nc(-c3ccccc3)n2)c1-c1ccccc1 ; [2H]c1cc([2H])c2sc3c([2H])c4c5c([2H])c([2H])c([2H])c([2H])c5n(-c5cccc(-c6nc(-c7ccccc7)nc(-c7ccccc7)n6)c5-c5ccccc5)c4c([2H])c3c2c1[2H]]

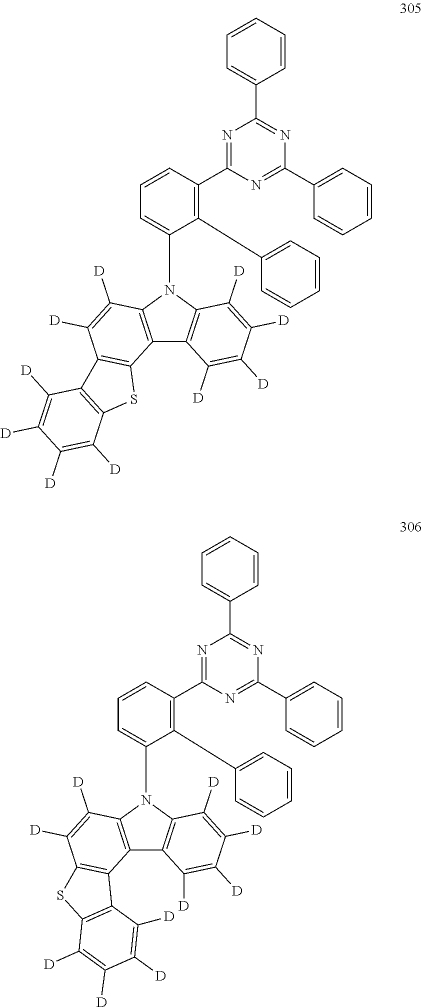 [SMILES: [2H]c1c([2H])c([2H])c2c(sc3c2c([2H])c([2H])c2c3c3c([2H])c([2H])c([2H])c([2H])c3n2-c2cccc(-c3nc(-c4ccccc4)nc(-c4ccccc4)n3)c2-c2ccccc2)c1[2H] ; [2H]c1c([2H])c([2H])c2c(sc3c([2H])c([2H])c4c(c5c([2H])c([2H])c([2H])c([2H])c5n4-c4cccc(-c5nc(-c6ccccc6)nc(-c6ccccc6)n5)c4-c4ccccc4)c32)c1[2H]]

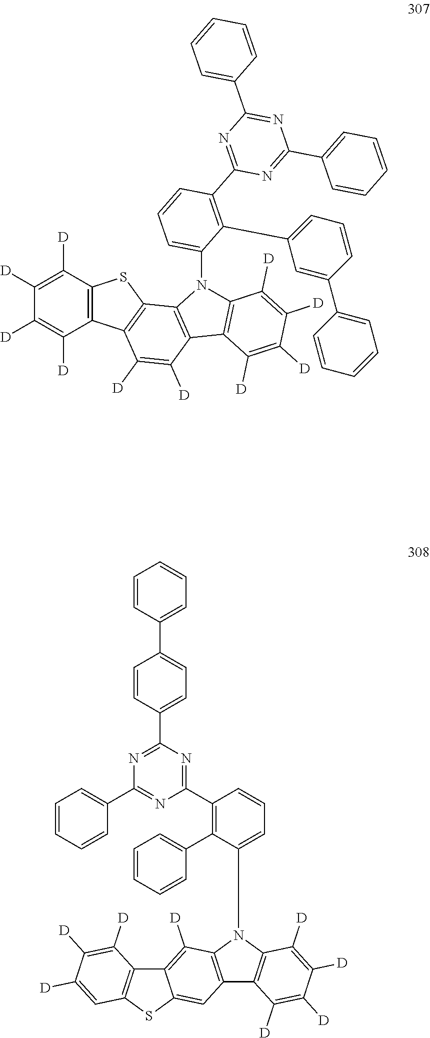 [SMILES: [2H]c1c([2H])c([2H])c2c(sc3c2c([2H])c([2H])c2c4c([2H])c([2H])c([2H])c([2H])c4n(-c4cccc(-c5nc(-c6ccccc6)nc(-c6ccccc6)n5)c4-c4cccc(-c5ccccc5)c4)c32)c1[2H] ; [2H]c1cc2sc3cc4c5c([2H])c([2H])c([2H])c([2H])c5n(-c5cccc(-c6nc(-c7ccccc7)nc(-c7ccc(-c8ccccc8)cc7)n6)c5-c5ccccc5)c4c([2H])c3c2c([2H])c1[2H]]

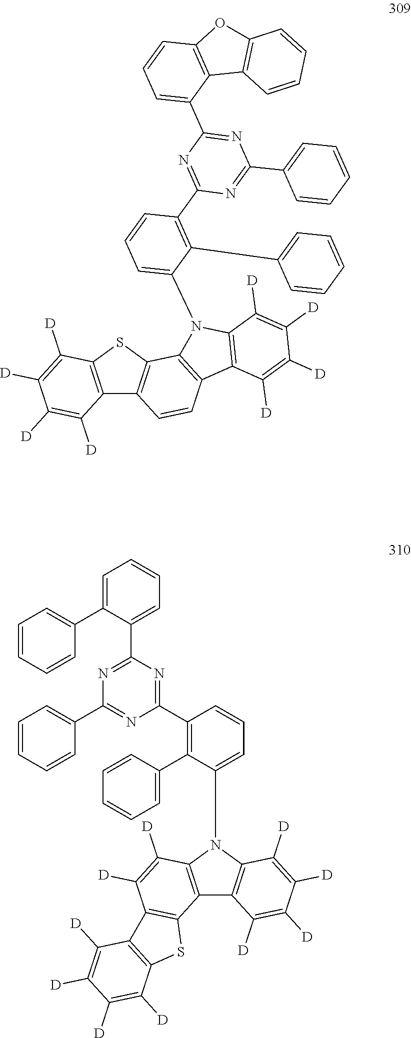 [SMILES: [2H]c1c([2H])c([2H])c2c(sc3c2ccc2c4c([2H])c([2H])c([2H])c([2H])c4n(-c4cccc(-c5nc(-c6ccccc6)nc(-c6cccc7oc8ccccc8c67)n5)c4-c4ccccc4)c23)c1[2H] ; [2H]c1c([2H])c([2H])c2c(sc3c2c([2H])c([2H])c2c3c3c([2H])c([2H])c([2H])c([2H])c3n2-c2cccc(-c3nc(-c4ccccc4)nc(-c4ccccc4-c4ccccc4)n3)c2-c2ccccc2)c1[2H]]

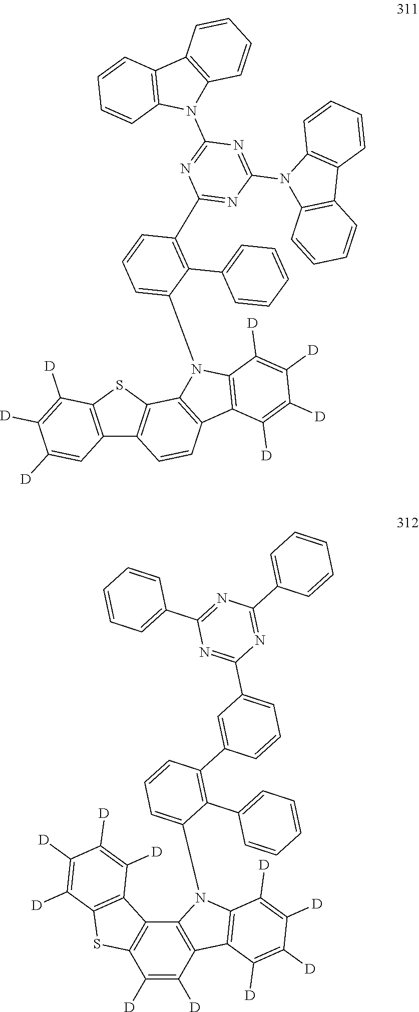 [SMILES: [2H]c1cc2c(sc3c2ccc2c4c([2H])c([2H])c([2H])c([2H])c4n(-c4cccc(-c5nc(-n6c7ccccc7c7ccccc76)nc(-n6c7ccccc7c7ccccc76)n5)c4-c4ccccc4)c23)c([2H])c1[2H] ; [2H]c1c([2H])c([2H])c2c(sc3c([2H])c([2H])c4c5c([2H])c([2H])c([2H])c([2H])c5n(-c5cccc(-c6cccc(-c7nc(-c8ccccc8)nc(-c8ccccc8)n7)c6)c5-c5ccccc5)c4c32)c1[2H]]

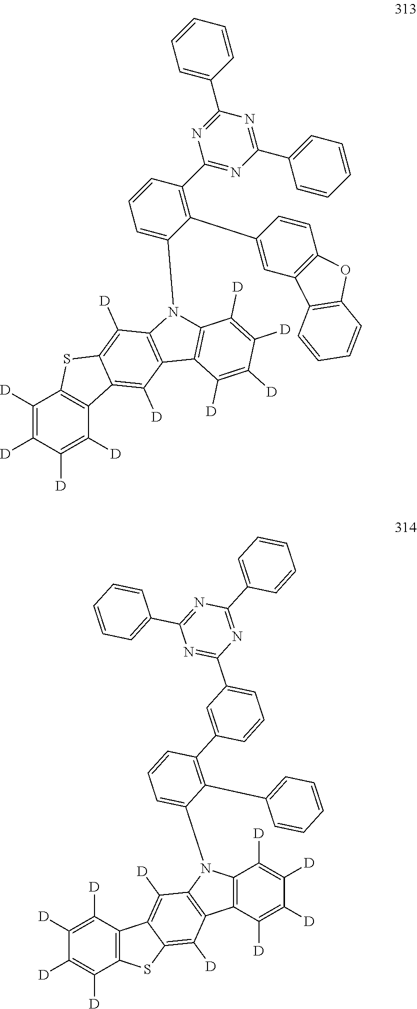 [SMILES: [2H]c1c([2H])c([2H])c2c(sc3c([2H])c4c(c([2H])c32)c2c([2H])c([2H])c([2H])c([2H])c2n4-c2cccc(-c3nc(-c4ccccc4)nc(-c4ccccc4)n3)c2-c2ccc3oc4ccccc4c3c2)c1[2H] ; [2H]c1c([2H])c([2H])c2c(sc3c([2H])c4c5c([2H])c([2H])c([2H])c([2H])c5n(-c5cccc(-c6cccc(-c7nc(-c8ccccc8)nc(-c8ccccc8)n7)c6)c5-c5ccccc5)c4c([2H])c32)c1[2H]]

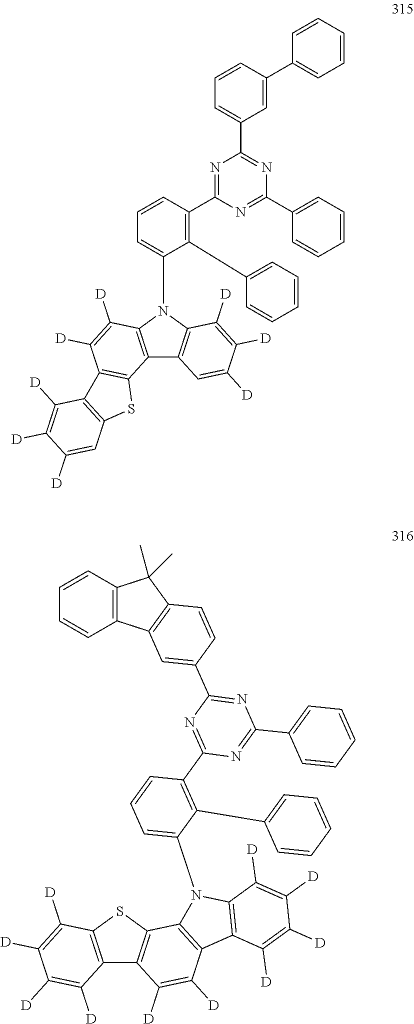 [SMILES: [2H]c1cc2sc3c(c([2H])c([2H])c4c3c3cc([2H])c([2H])c([2H])c3n4-c3cccc(-c4nc(-c5ccccc5)nc(-c5cccc(-c6ccccc6)c5)n4)c3-c3ccccc3)c2c([2H])c1[2H] ; [2H]c1c([2H])c([2H])c2c(sc3c2c([2H])c([2H])c2c4c([2H])c([2H])c([2H])c([2H])c4n(-c4cccc(-c5nc(-c6ccccc6)nc(-c6ccc7c(c6)-c6ccccc6C7(C)C)n5)c4-c4ccccc4)c32)c1[2H]]

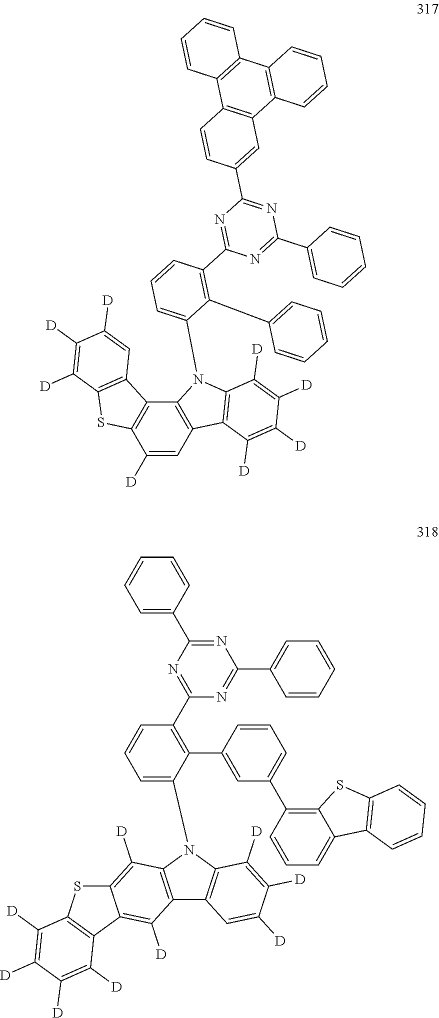 [SMILES: [2H]c1cc2c(sc3c([2H])cc4c5c([2H])c([2H])c([2H])c([2H])c5n(-c5cccc(-c6nc(-c7ccccc7)nc(-c7ccc8c9ccccc9c9ccccc9c8c7)n6)c5-c5ccccc5)c4c32)c([2H])c1[2H] ; [2H]c1cc2c3c([2H])c4c(sc5c([2H])c([2H])c([2H])c([2H])c54)c([2H])c3n(-c3cccc(-c4nc(-c5ccccc5)nc(-c5ccccc5)n4)c3-c3cccc(-c4cccc5c4sc4ccccc45)c3)c2c([2H])c1[2H]]

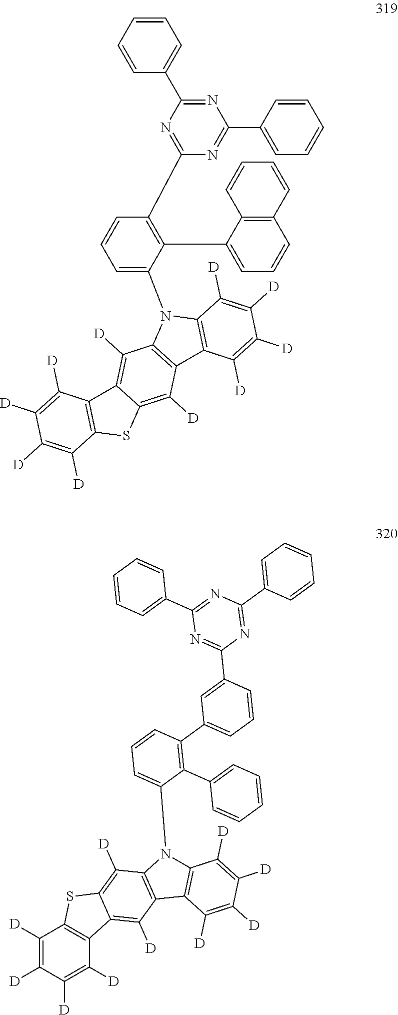

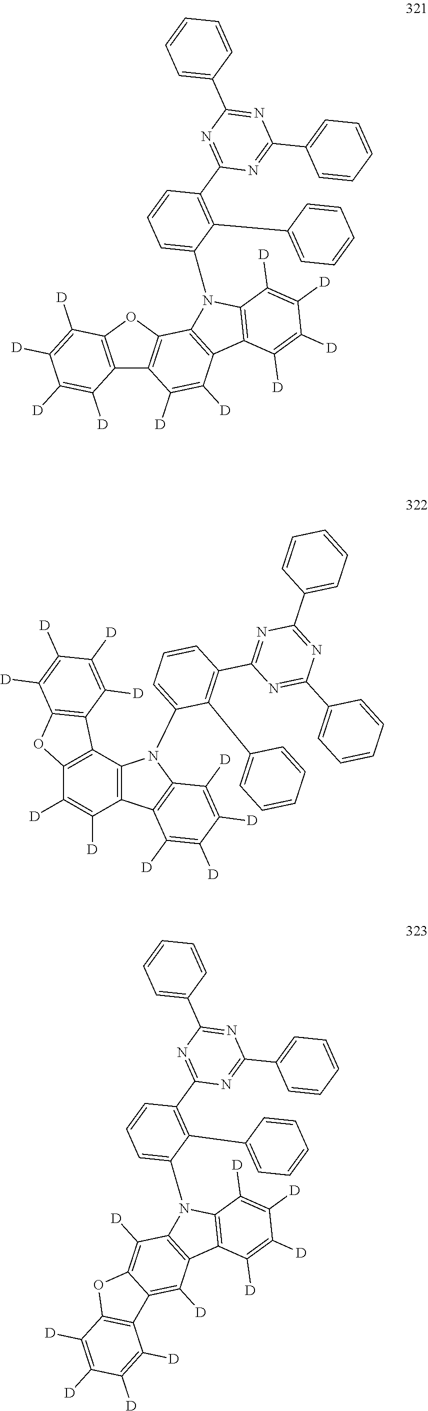 [SMILES: [2H]c1c([2H])c([2H])c2c(oc3c2c([2H])c([2H])c2c4c([2H])c([2H])c([2H])c([2H])c4n(-c4cccc(-c5nc(-c6ccccc6)nc(-c6ccccc6)n5)c4-c4ccccc4)c32)c1[2H] ; [2H]c1c([2H])c([2H])c2c(oc3c([2H])c([2H])c4c5c([2H])c([2H])c([2H])c([2H])c5n(-c5cccc(-c6nc(-c7ccccc7)nc(-c7ccccc7)n6)c5-c5ccccc5)c4c32)c1[2H] ; [2H]c1c([2H])c([2H])c2c(oc3c([2H])c4c(c([2H])c32)c2c([2H])c([2H])c([2H])c([2H])c2n4-c2cccc(-c3nc(-c4ccccc4)nc(-c4ccccc4)n3)c2-c2ccccc2)c1[2H]]

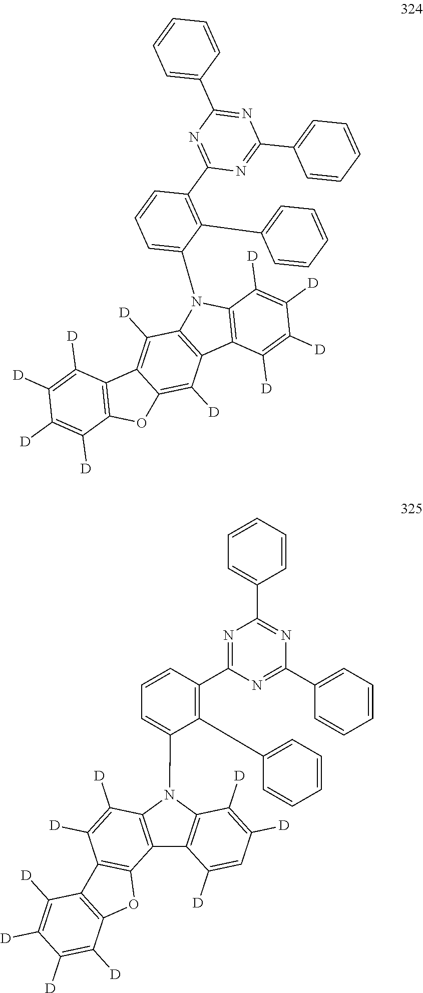 [SMILES: [2H]c1c([2H])c([2H])c2c(oc3c([2H])c4c5c([2H])c([2H])c([2H])c([2H])c5n(-c5cccc(-c6nc(-c7ccccc7)nc(-c7ccccc7)n6)c5-c5ccccc5)c4c([2H])c32)c1[2H] ; [2H]c1cc([2H])c2c3c4oc5c([2H])c([2H])c([2H])c([2H])c5c4c([2H])c([2H])c3n(-c3cccc(-c4nc(-c5ccccc5)nc(-c5ccccc5)n4)c3-c3ccccc3)c2c1[2H]]

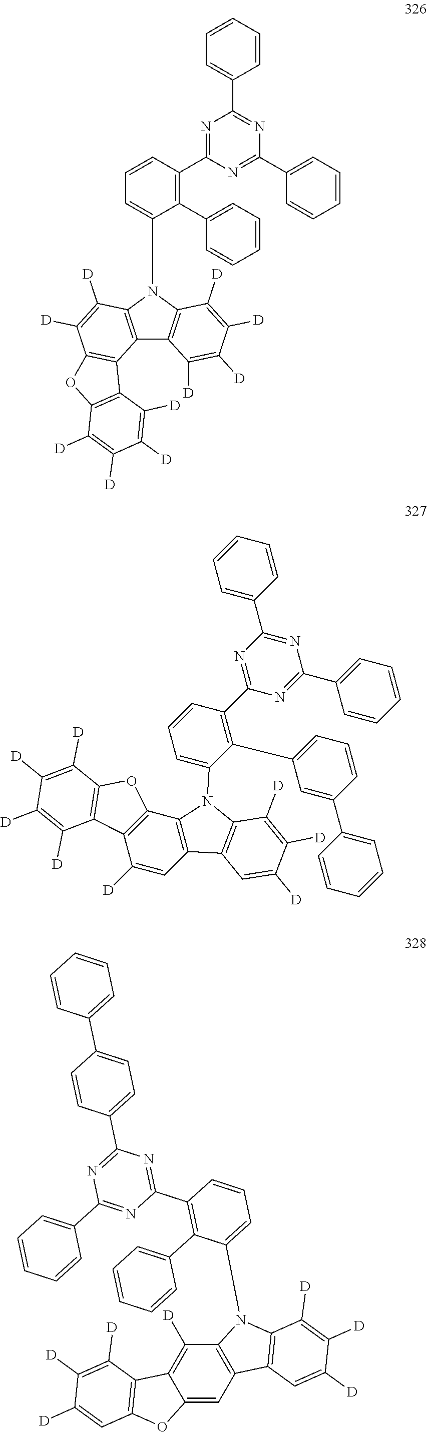 [SMILES: [2H]c1c([2H])c([2H])c2c(oc3c([2H])c([2H])c4c(c5c([2H])c([2H])c([2H])c([2H])c5n4-c4cccc(-c5nc(-c6ccccc6)nc(-c6ccccc6)n5)c4-c4ccccc4)c32)c1[2H] ; [2H]c1cc2c3cc([2H])c4c(oc5c([2H])c([2H])c([2H])c([2H])c54)c3n(-c3cccc(-c4nc(-c5ccccc5)nc(-c5ccccc5)n4)c3-c3cccc(-c4ccccc4)c3)c2c([2H])c1[2H] ; [2H]c1cc2oc3cc4c5cc([2H])c([2H])c([2H])c5n(-c5cccc(-c6nc(-c7ccccc7)nc(-c7ccc(-c8ccccc8)cc7)n6)c5-c5ccccc5)c4c([2H])c3c2c([2H])c1[2H]]

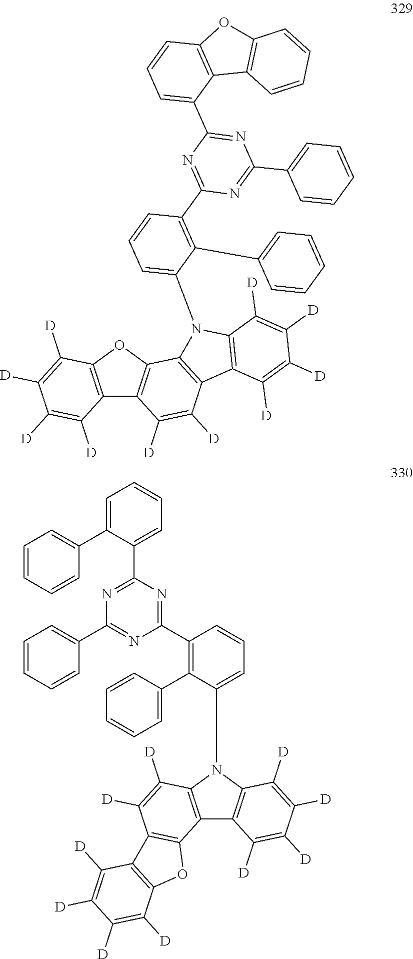

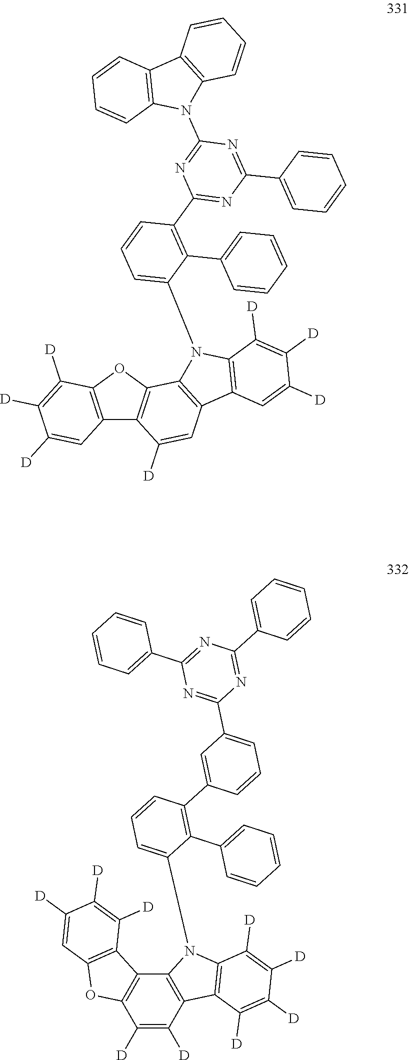 [SMILES: [2H]c1cc2c(oc3c2c([2H])cc2c4cc([2H])c([2H])c([2H])c4n(-c4cccc(-c5nc(-c6ccccc6)nc(-n6c7ccccc7c7ccccc76)n5)c4-c4ccccc4)c23)c([2H])c1[2H] ; [2H]c1cc2oc3c([2H])c([2H])c4c5c([2H])c([2H])c([2H])c([2H])c5n(-c5cccc(-c6cccc(-c7nc(-c8ccccc8)nc(-c8ccccc8)n7)c6)c5-c5ccccc5)c4c3c2c([2H])c1[2H]]

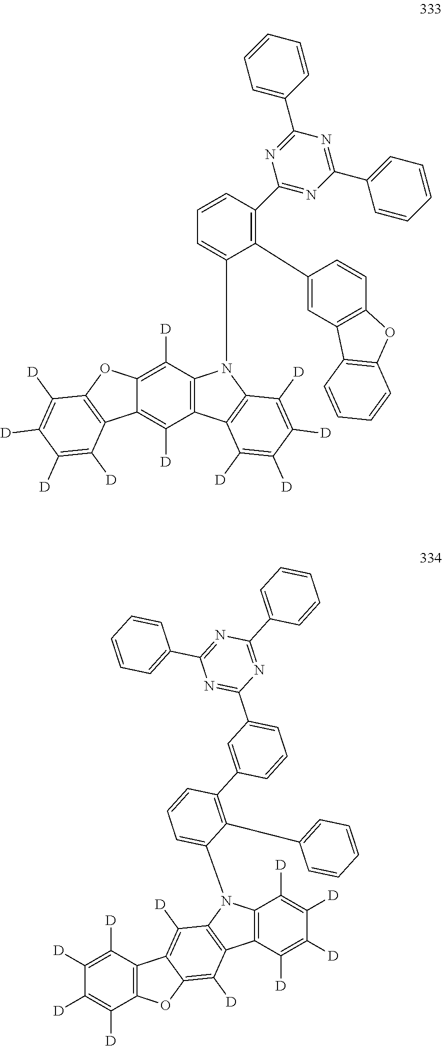 [SMILES: [2H]c1c([2H])c([2H])c2c(oc3c([2H])c4c(c([2H])c32)c2c([2H])c([2H])c([2H])c([2H])c2n4-c2cccc(-c3nc(-c4ccccc4)nc(-c4ccccc4)n3)c2-c2ccc3oc4ccccc4c3c2)c1[2H] ; [2H]c1c([2H])c([2H])c2c(oc3c([2H])c4c5c([2H])c([2H])c([2H])c([2H])c5n(-c5cccc(-c6cccc(-c7nc(-c8ccccc8)nc(-c8ccccc8)n7)c6)c5-c5ccccc5)c4c([2H])c32)c1[2H]]

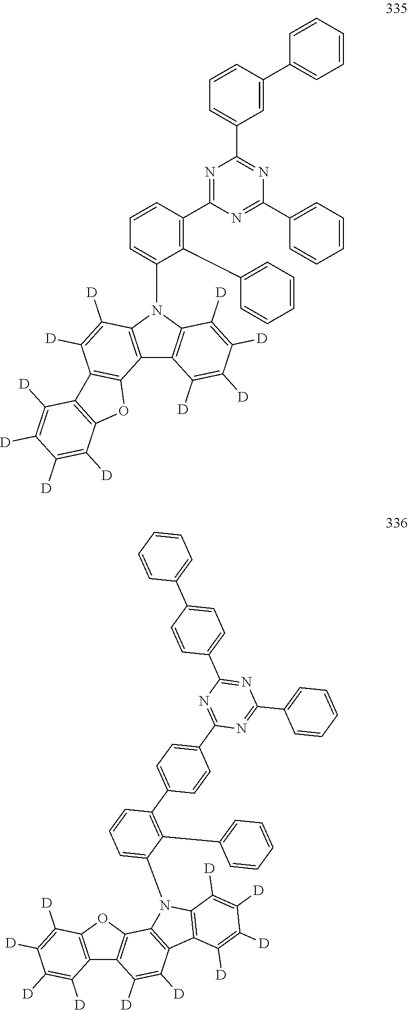 [SMILES: [2H]c1c([2H])c([2H])c2c(oc3c2c([2H])c([2H])c2c3c3c([2H])c([2H])c([2H])c([2H])c3n2-c2cccc(-c3nc(-c4ccccc4)nc(-c4cccc(-c5ccccc5)c4)n3)c2-c2ccccc2)c1[2H] ; [2H]c1c([2H])c([2H])c2c(oc3c2c([2H])c([2H])c2c4c([2H])c([2H])c([2H])c([2H])c4n(-c4cccc(-c5ccc(-c6nc(-c7ccccc7)nc(-c7ccc(-c8ccccc8)cc7)n6)cc5)c4-c4ccccc4)c32)c1[2H]]

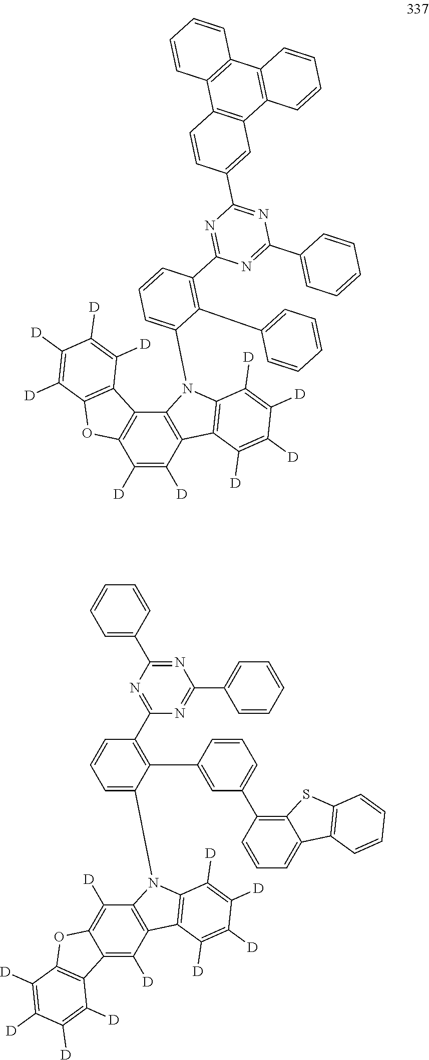 [SMILES: [2H]c1c([2H])c([2H])c2c(oc3c([2H])c([2H])c4c5c([2H])c([2H])c([2H])c([2H])c5n(-c5cccc(-c6nc(-c7ccccc7)nc(-c7ccc8c9ccccc9c9ccccc9c8c7)n6)c5-c5ccccc5)c4c32)c1[2H] ; [2H]c1c([2H])c([2H])c2c(oc3c([2H])c4c(c([2H])c32)c2c([2H])c([2H])c([2H])c([2H])c2n4-c2cccc(-c3nc(-c4ccccc4)nc(-c4ccccc4)n3)c2-c2cccc(-c3cccc4c3sc3ccccc34)c2)c1[2H]]

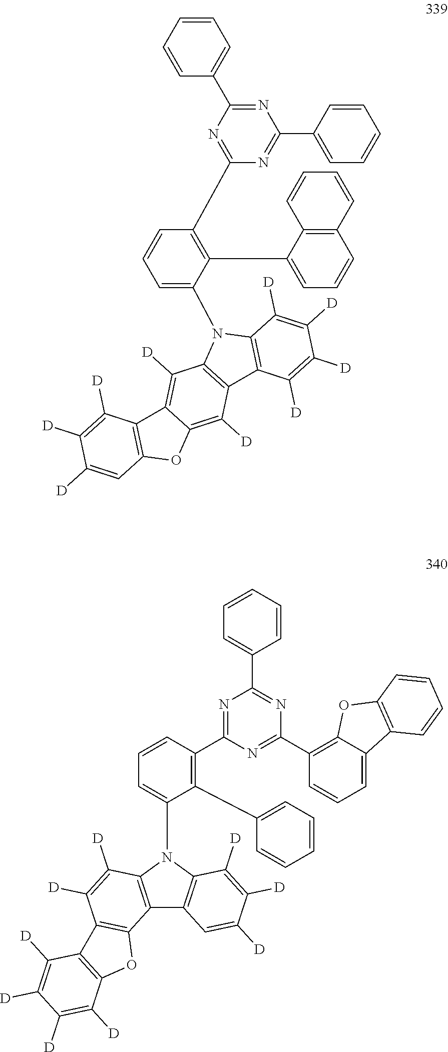 [SMILES: [2H]c1cc2oc3c([2H])c4c5c([2H])c([2H])c([2H])c([2H])c5n(-c5cccc(-c6nc(-c7ccccc7)nc(-c7ccccc7)n6)c5-c5cccc6ccccc56)c4c([2H])c3c2c([2H])c1[2H] ; [2H]c1cc2c3c4oc5c([2H])c([2H])c([2H])c([2H])c5c4c([2H])c([2H])c3n(-c3cccc(-c4nc(-c5ccccc5)nc(-c5cccc6c5oc5ccccc56)n4)c3-c3ccccc3)c2c([2H])c1[2H]]

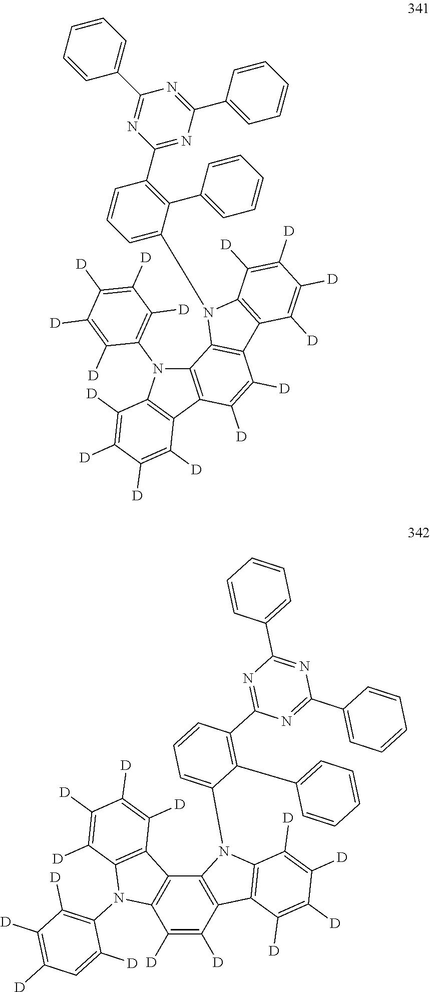 [SMILES: [2H]c1c([2H])c([2H])c(-n2c3c([2H])c([2H])c([2H])c([2H])c3c3c([2H])c([2H])c4c5c([2H])c([2H])c([2H])c([2H])c5n(-c5cccc(-c6nc(-c7ccccc7)nc(-c7ccccc7)n6)c5-c5ccccc5)c4c32)c([2H])c1[2H] ; [2H]c1cc([2H])c(-n2c3c([2H])c([2H])c([2H])c([2H])c3c3c2c([2H])c([2H])c2c4c([2H])c([2H])c([2H])c([2H])c4n(-c4cccc(-c5nc(-c6ccccc6)nc(-c6ccccc6)n5)c4-c4ccccc4)c23)c([2H])c1[2H]]

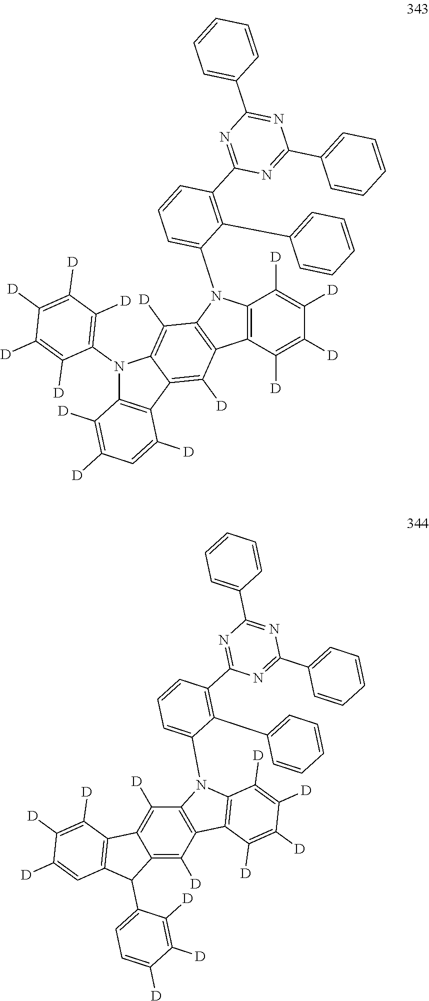 [SMILES: [2H]c1cc([2H])c2c3c([2H])c4c5c([2H])c([2H])c([2H])c([2H])c5n(-c5cccc(-c6nc(-c7ccccc7)nc(-c7ccccc7)n6)c5-c5ccccc5)c4c([2H])c3n(-c3c([2H])c([2H])c([2H])c([2H])c3[2H])c2c1[2H] ; [2H]c1ccc(C2c3cc([2H])c([2H])c([2H])c3-c3c2c([2H])c2c4c([2H])c([2H])c([2H])c([2H])c4n(-c4cccc(-c5nc(-c6ccccc6)nc(-c6ccccc6)n5)c4-c4ccccc4)c2c3[2H])c([2H])c1[2H]]

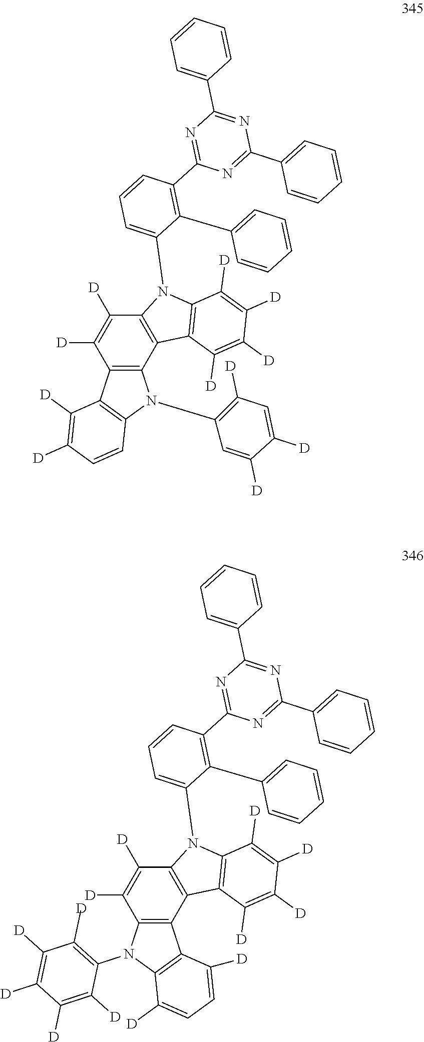

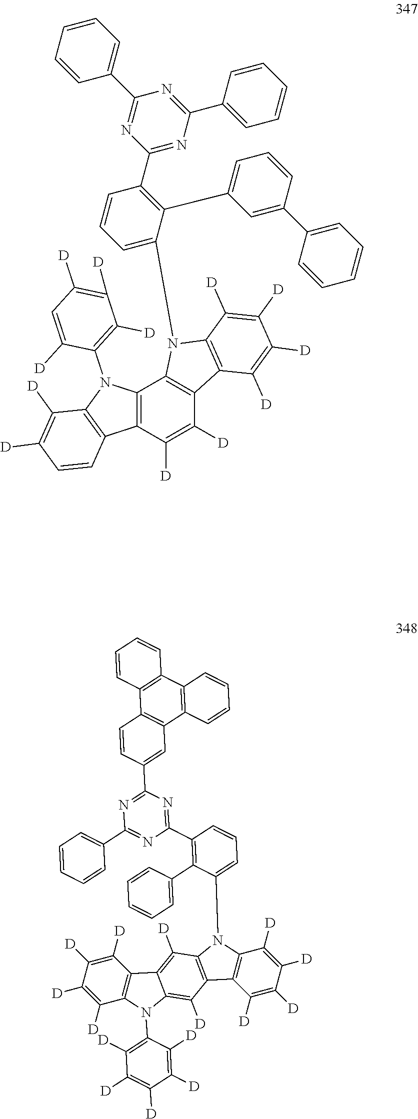 [SMILES: [2H]c1cc([2H])c(-n2c3c([2H])c([2H])ccc3c3c([2H])c([2H])c4c5c([2H])c([2H])c([2H])c([2H])c5n(-c5cccc(-c6nc(-c7ccccc7)nc(-c7ccccc7)n6)c5-c5cccc(-c6ccccc6)c5)c4c32)c([2H])c1[2H] ; [2H]c1c([2H])c([2H])c(-n2c3c([2H])c([2H])c([2H])c([2H])c3c3c([2H])c4c(c([2H])c32)c2c([2H])c([2H])c([2H])c([2H])c2n4-c2cccc(-c3nc(-c4ccccc4)nc(-c4ccc5c6ccccc6c6ccccc6c5c4)n3)c2-c2ccccc2)c([2H])c1[2H]]

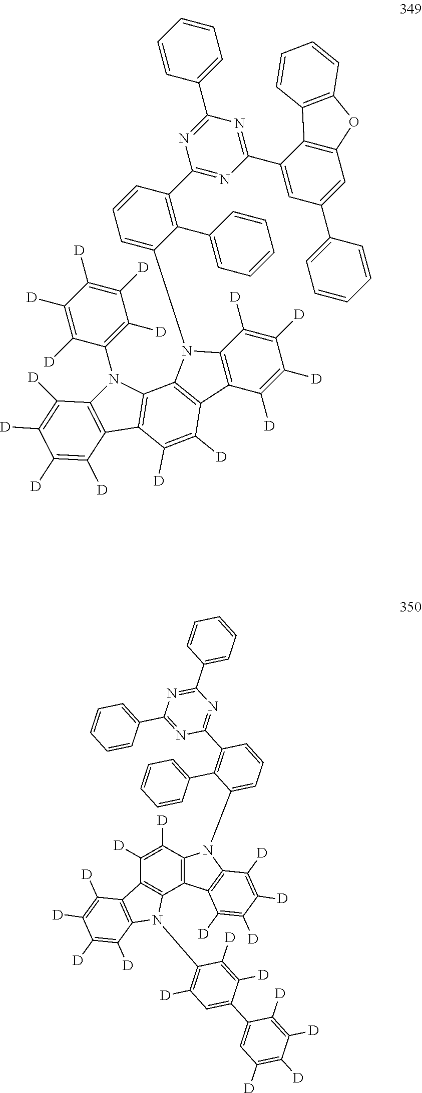 [SMILES: [2H]c1c([2H])c([2H])c(-n2c3c([2H])c([2H])c([2H])c([2H])c3c3c([2H])c([2H])c4c5c([2H])c([2H])c([2H])c([2H])c5n(-c5cccc(-c6nc(-c7ccccc7)nc(-c7cc(-c8ccccc8)cc8oc9ccccc9c78)n6)c5-c5ccccc5)c4c32)c([2H])c1[2H] ; [2H]c1cc(-c2cc([2H])c(-n3c4c([2H])c([2H])c([2H])c([2H])c4c4c([2H])c([2H])c5c(c6c([2H])c([2H])c([2H])c([2H])c6n5-c5cccc(-c6nc(-c7ccccc7)nc(-c7ccccc7)n6)c5-c5ccccc5)c43)c([2H])c2[2H])c([2H])c([2H])c1[2H]]

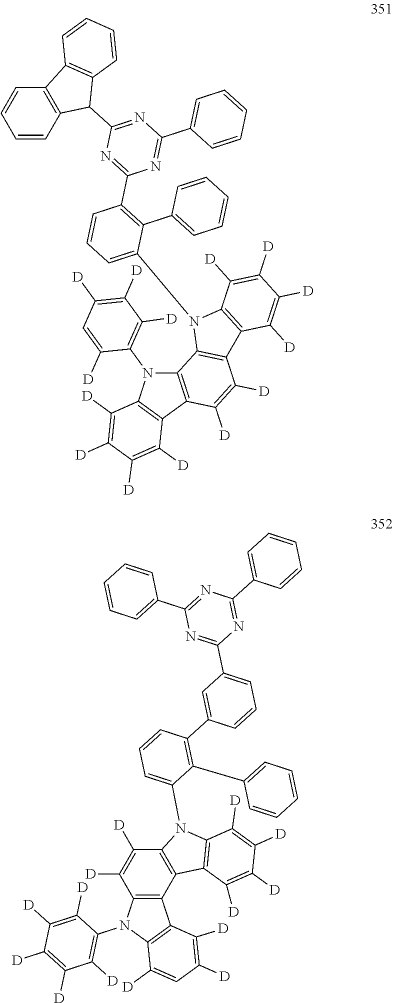 [SMILES: [2H]c1cc([2H])c(-n2c3c([2H])c([2H])c([2H])c([2H])c3c3c([2H])c([2H])c4c5c([2H])c([2H])c([2H])c([2H])c5n(-c5cccc(-c6nc(-c7ccccc7)nc(C7c8ccccc8-c8ccccc87)n6)c5-c5ccccc5)c4c32)c([2H])c1[2H] ; [2H]c1cc([2H])c2c(c1[2H])c1c3c4c([2H])c([2H])c([2H])c([2H])c4n(-c4cccc(-c5cccc(-c6nc(-c7ccccc7)nc(-c7ccccc7)n6)c5)c4-c4ccccc4)c3c([2H])c([2H])c1n2-c1c([2H])c([2H])c([2H])c([2H])c1[2H]]

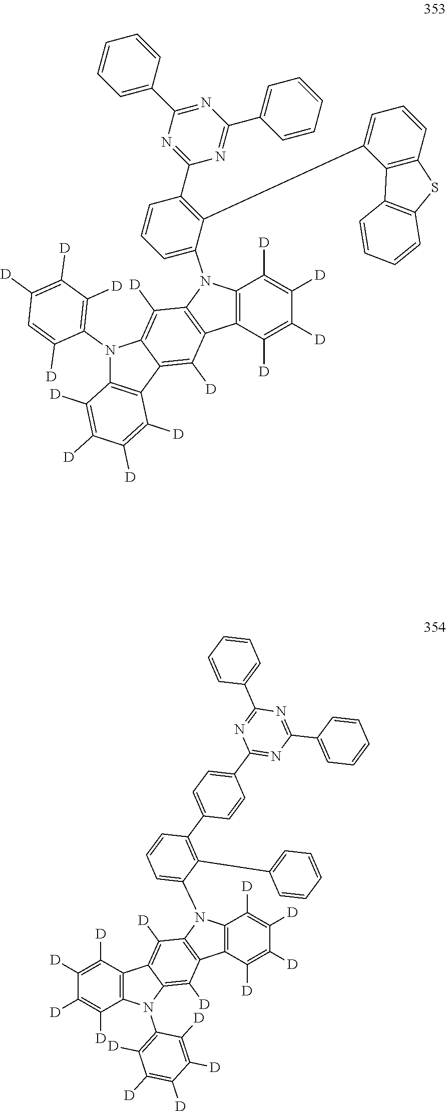 [SMILES: [2H]c1cc([2H])c(-n2c3c([2H])c([2H])c([2H])c([2H])c3c3c([2H])c4c5c([2H])c([2H])c([2H])c([2H])c5n(-c5cccc(-c6nc(-c7ccccc7)nc(-c7ccccc7)n6)c5-c5cccc6sc7ccccc7c56)c4c([2H])c32)c([2H])c1[2H] ; [2H]c1c([2H])c([2H])c(-n2c3c([2H])c([2H])c([2H])c([2H])c3c3c([2H])c4c(c([2H])c32)c2c([2H])c([2H])c([2H])c([2H])c2n4-c2cccc(-c3ccc(-c4nc(-c5ccccc5)nc(-c5ccccc5)n4)cc3)c2-c2ccccc2)c([2H])c1[2H]]

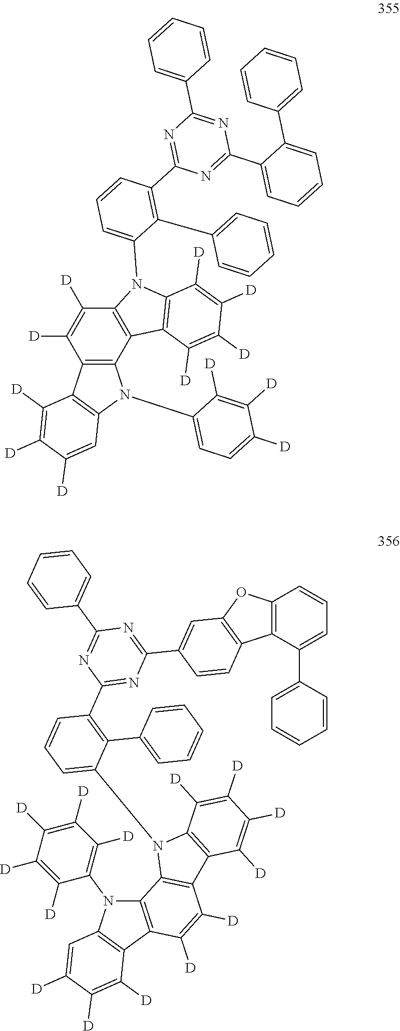 [SMILES: [2H]c1ccc(-n2c3cc([2H])c([2H])c([2H])c3c3c([2H])c([2H])c4c(c5c([2H])c([2H])c([2H])c([2H])c5n4-c4cccc(-c5nc(-c6ccccc6)nc(-c6ccccc6-c6ccccc6)n5)c4-c4ccccc4)c32)c([2H])c1[2H] ; [2H]c1cc2c(c([2H])c1[2H])c1c([2H])c([2H])c3c4c([2H])c([2H])c([2H])c([2H])c4n(-c4cccc(-c5nc(-c6ccccc6)nc(-c6ccc7c(c6)oc6cccc(-c8ccccc8)c67)n5)c4-c4ccccc4)c3c1n2-c1c([2H])c([2H])c([2H])c([2H])c1[2H]]

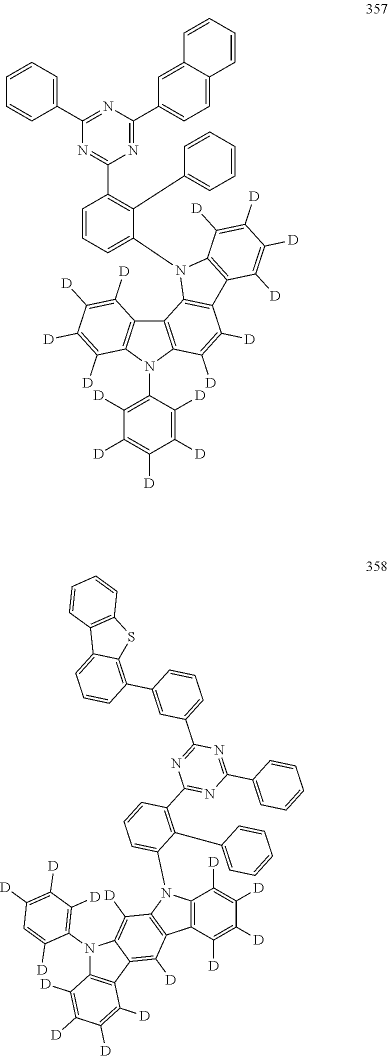 [SMILES: [2H]c1c([2H])c([2H])c(-n2c3c([2H])c([2H])c([2H])c([2H])c3c3c2c([2H])c([2H])c2c4c([2H])c([2H])c([2H])c([2H])c4n(-c4cccc(-c5nc(-c6ccccc6)nc(-c6ccc7ccccc7c6)n5)c4-c4ccccc4)c23)c([2H])c1[2H] ; [2H]c1cc([2H])c(-n2c3c([2H])c([2H])c([2H])c([2H])c3c3c([2H])c4c5c([2H])c([2H])c([2H])c([2H])c5n(-c5cccc(-c6nc(-c7ccccc7)nc(-c7cccc(-c8cccc9c8sc8ccccc89)c7)n6)c5-c5ccccc5)c4c([2H])c32)c([2H])c1[2H]]

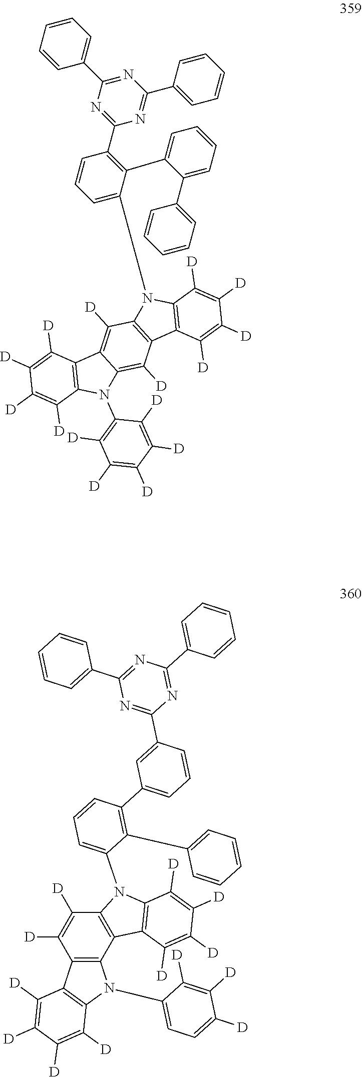 [SMILES: [2H]c1c([2H])c([2H])c(-n2c3c([2H])c([2H])c([2H])c([2H])c3c3c([2H])c4c(c([2H])c32)c2c([2H])c([2H])c([2H])c([2H])c2n4-c2cccc(-c3nc(-c4ccccc4)nc(-c4ccccc4)n3)c2-c2ccccc2-c2ccccc2)c([2H])c1[2H] ; [2H]c1ccc(-n2c3c([2H])c([2H])c([2H])c([2H])c3c3c([2H])c([2H])c4c(c5c([2H])c([2H])c([2H])c([2H])c5n4-c4cccc(-c5cccc(-c6nc(-c7ccccc7)nc(-c7ccccc7)n6)c5)c4-c4ccccc4)c32)c([2H])c1[2H]]

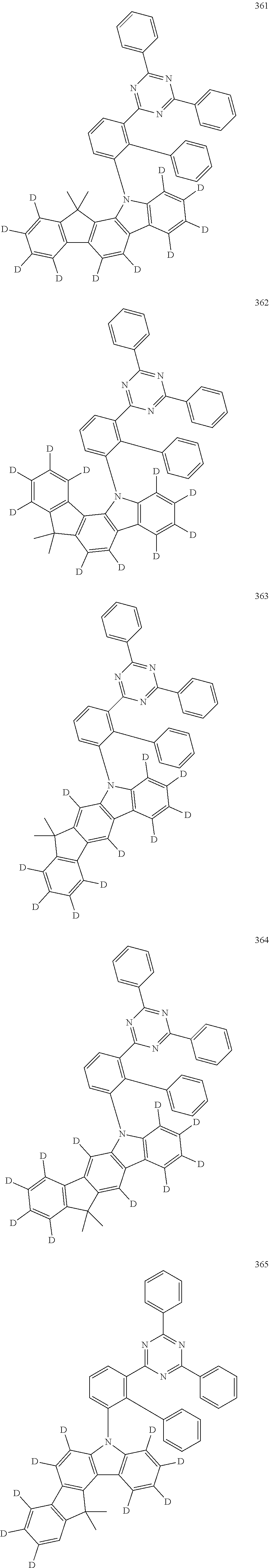

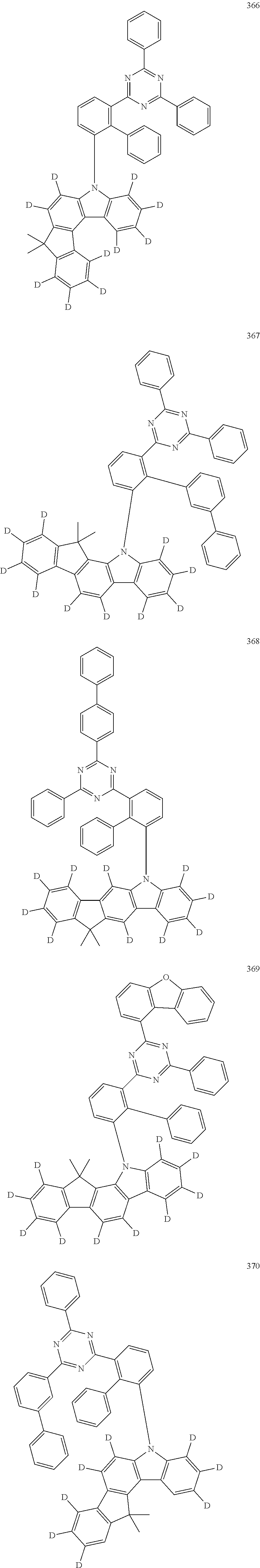

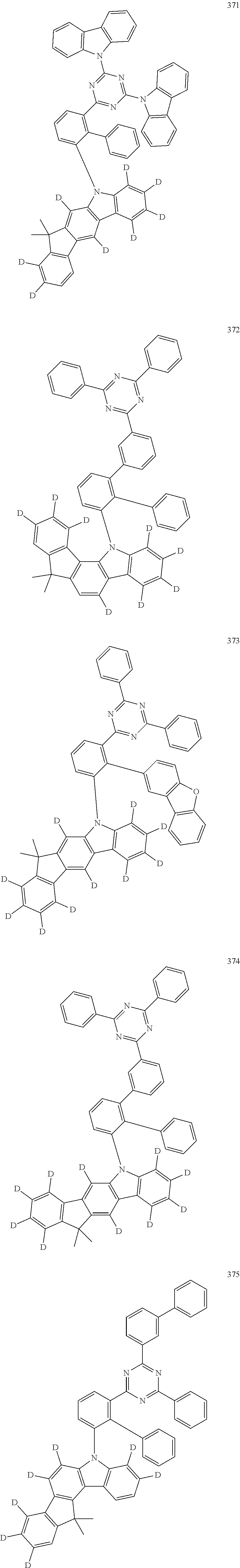

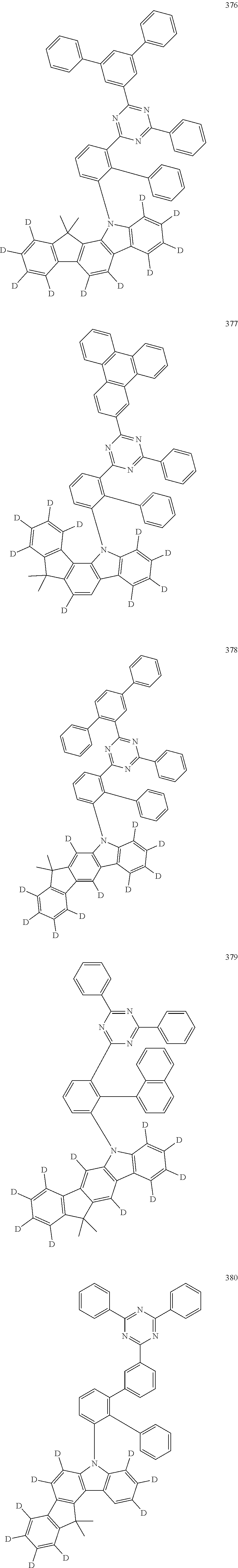

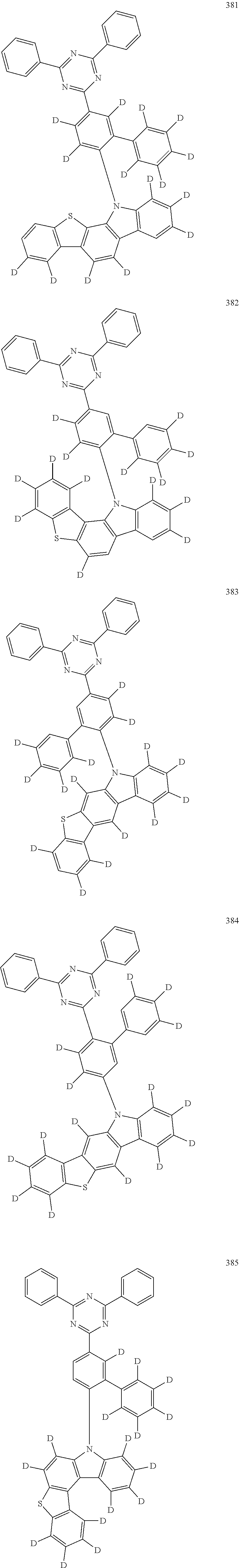

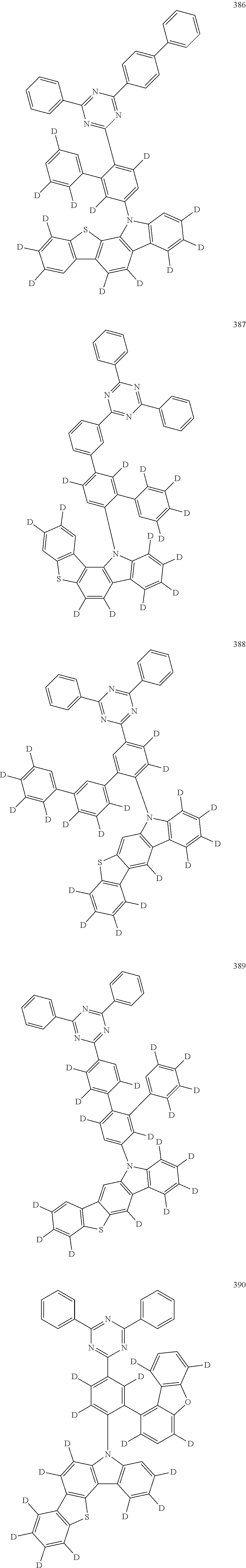

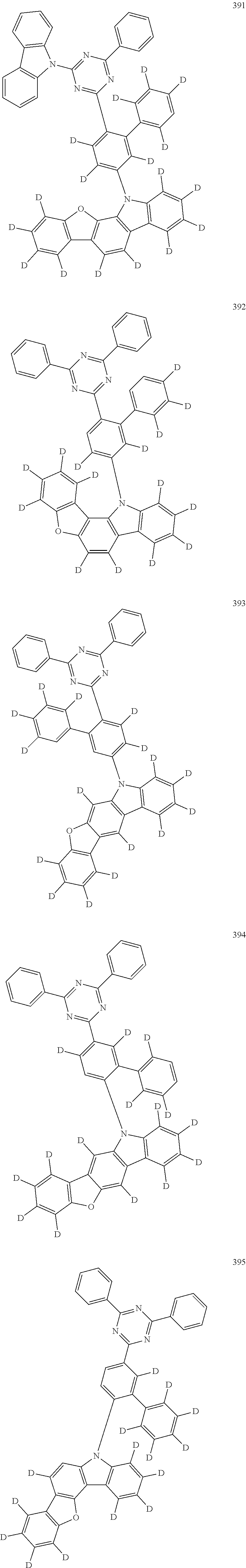

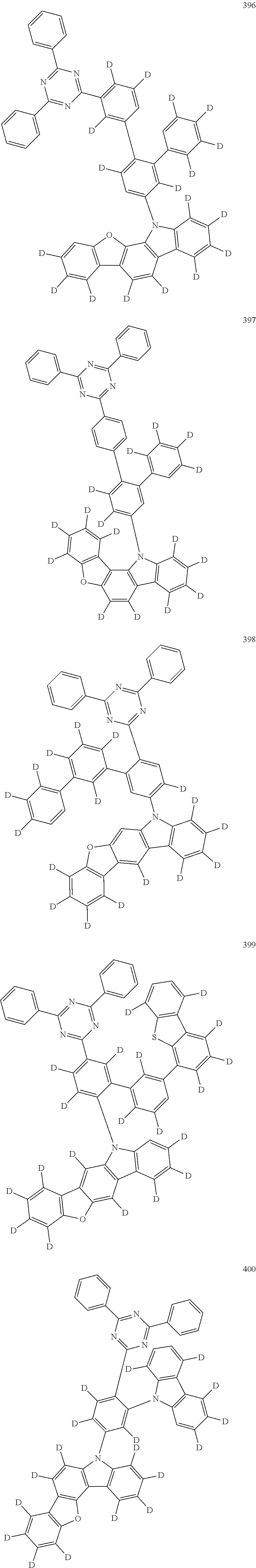

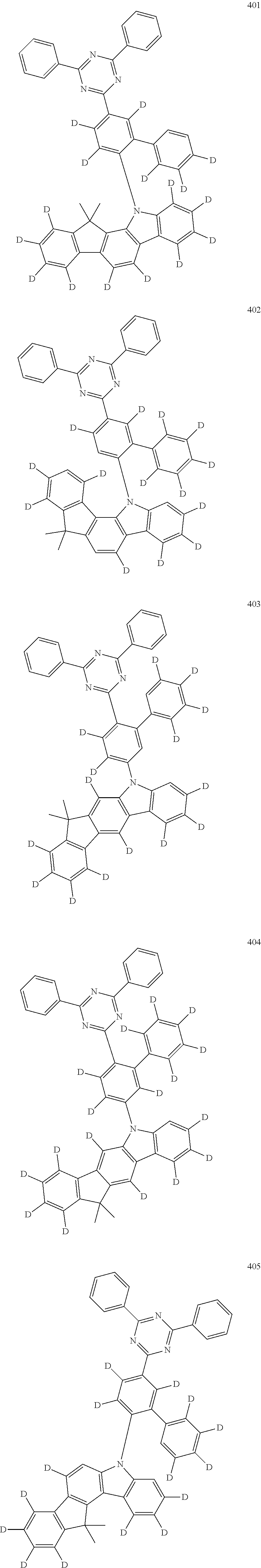

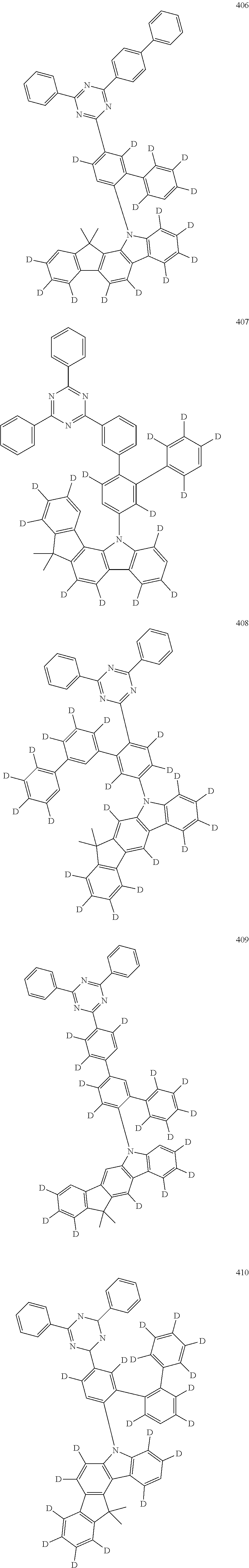

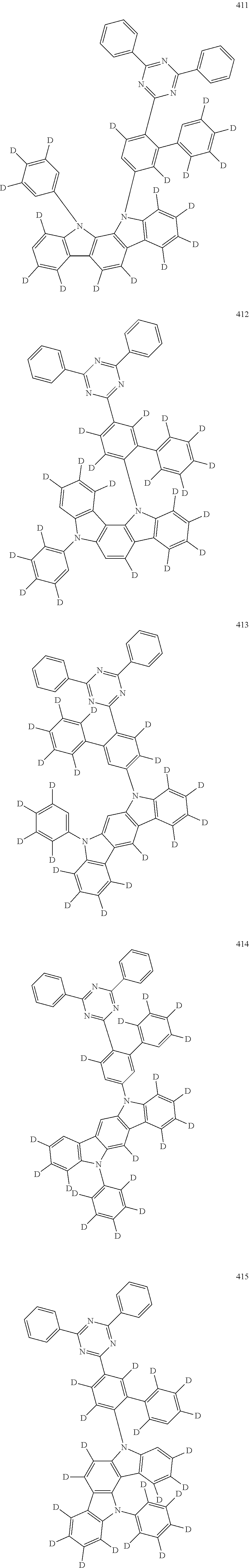

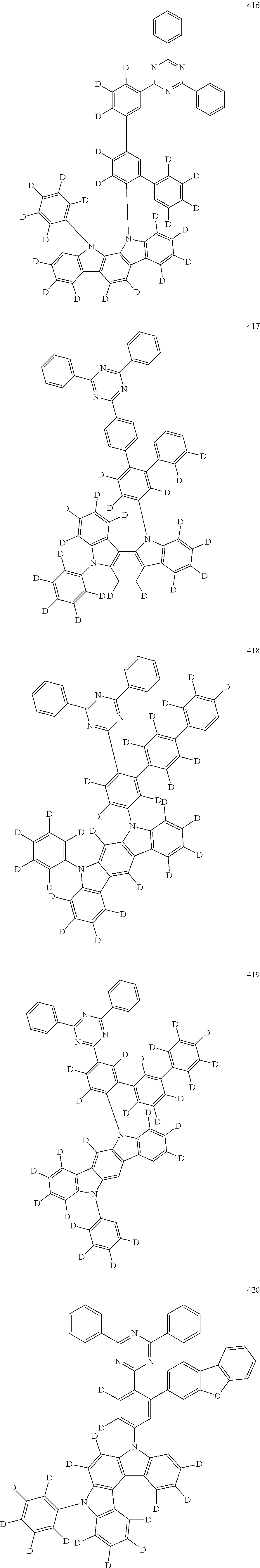

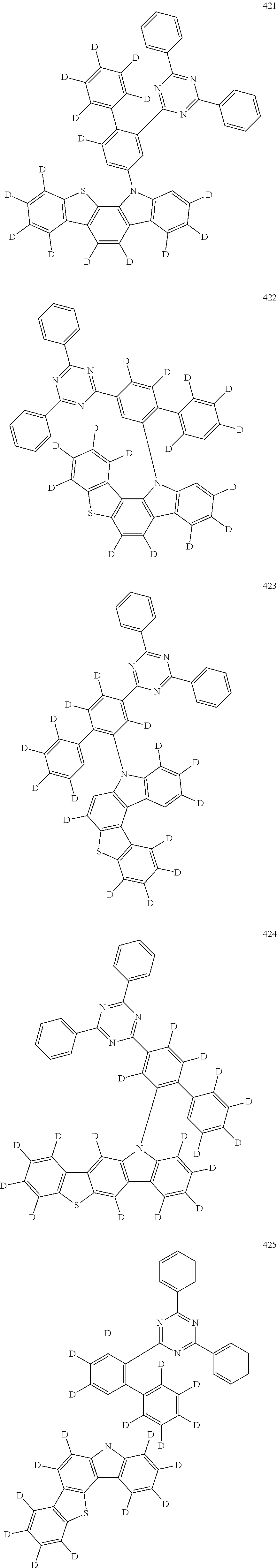

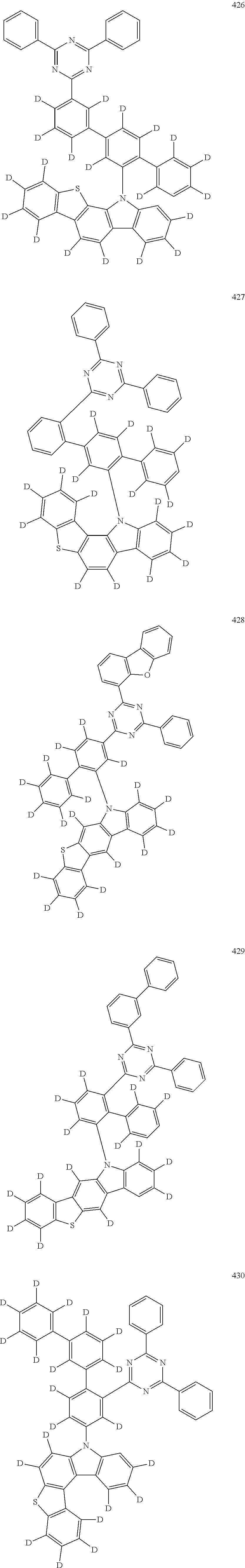

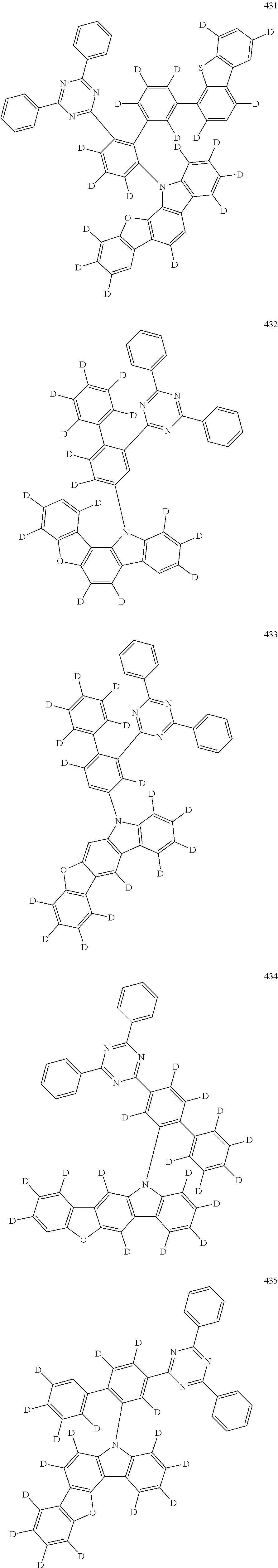

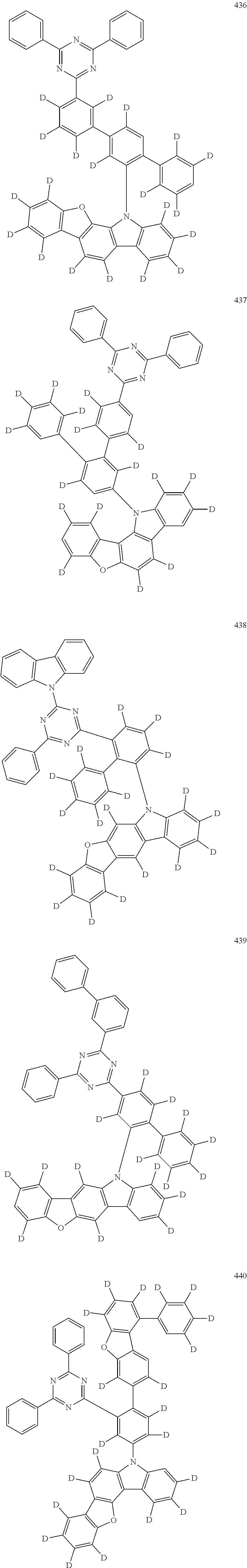

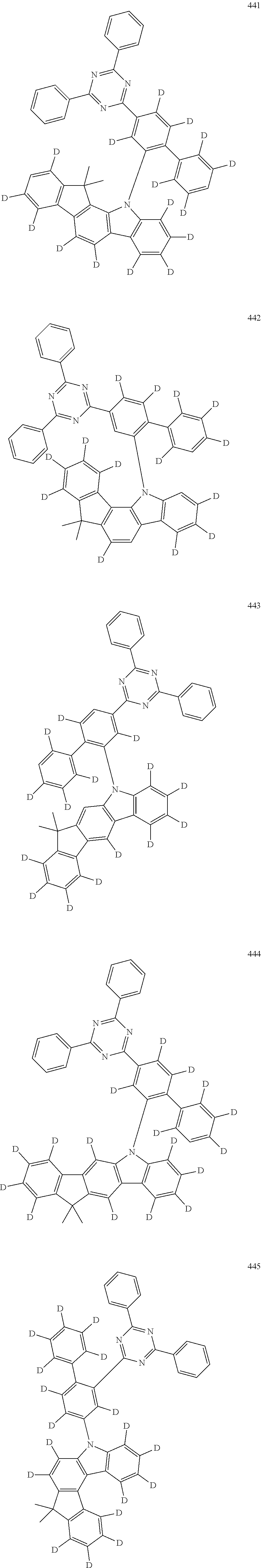

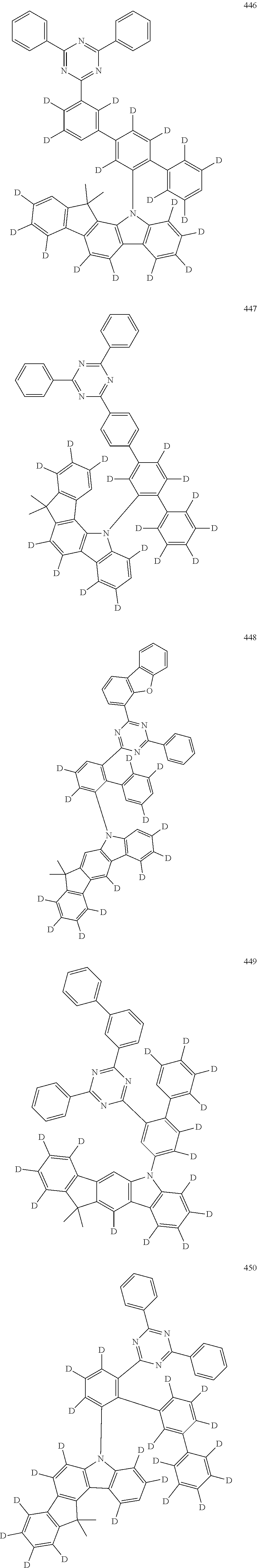

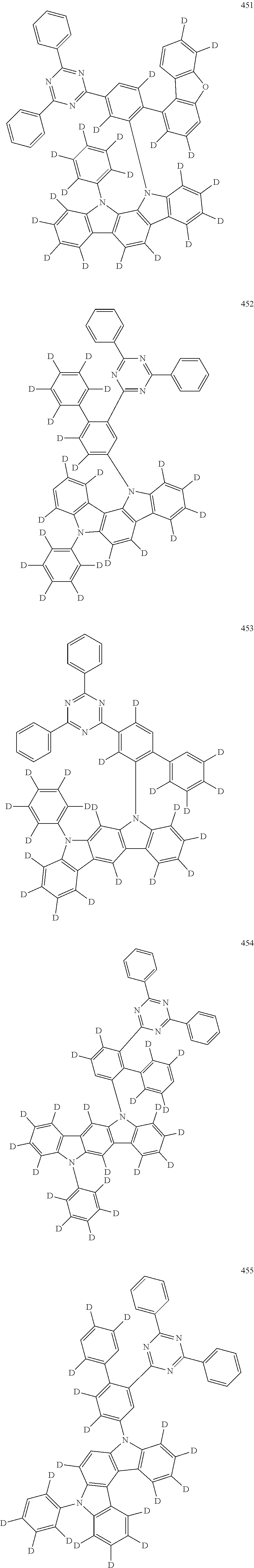

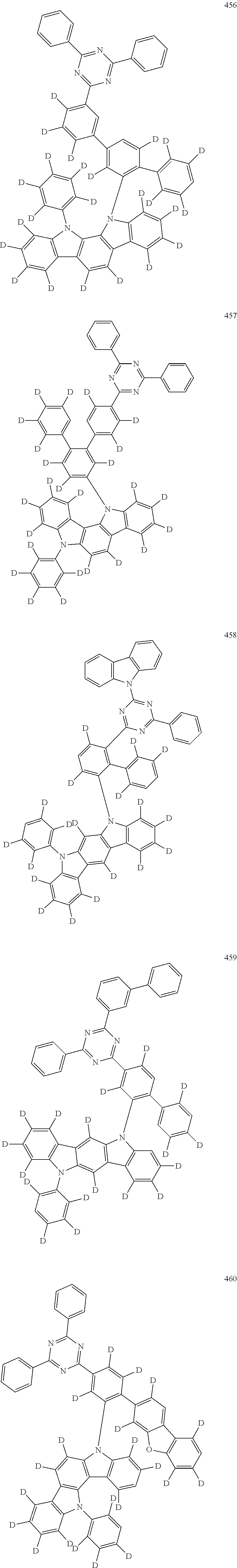

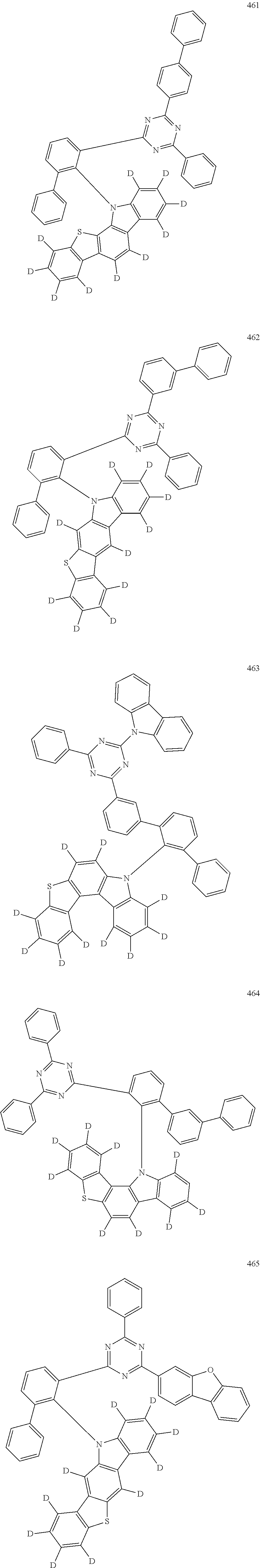

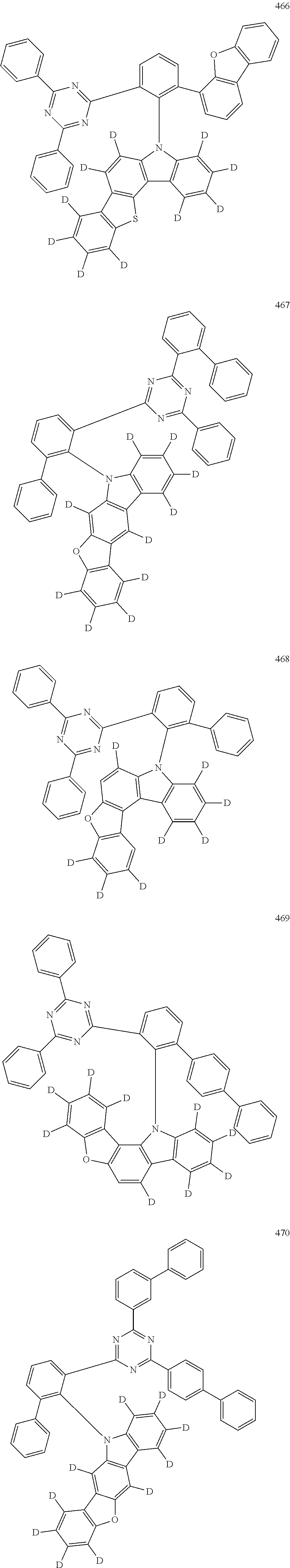

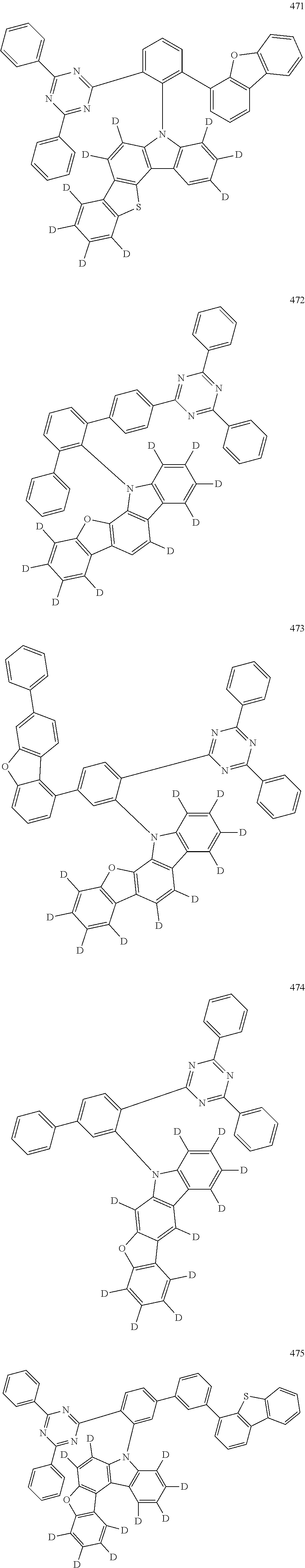

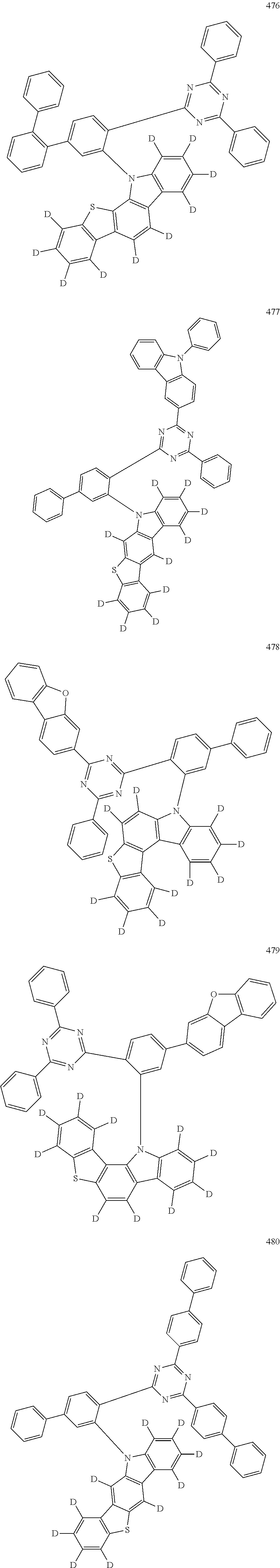

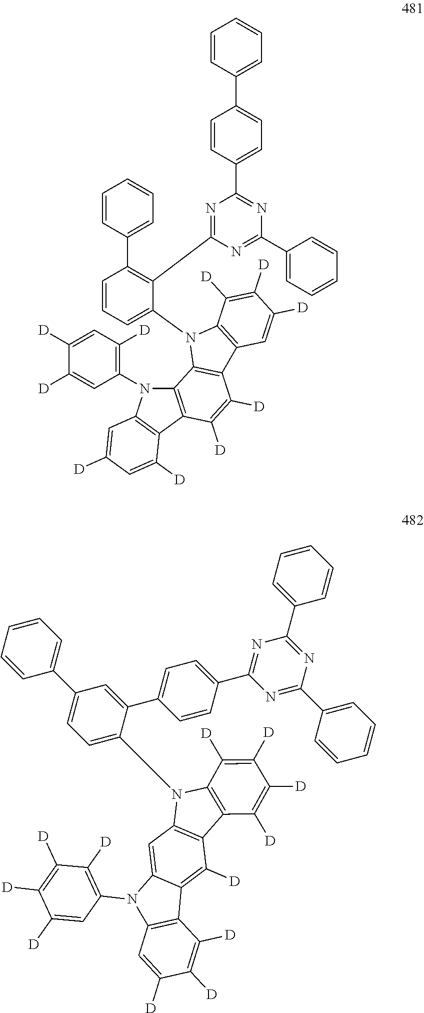 [SMILES: [2H]c1cc([2H])c2c3c([2H])c([2H])c4c5cc([2H])c([2H])c([2H])c5n(-c5cccc(-c6ccccc6)c5-c5nc(-c6ccccc6)nc(-c6ccc(-c7ccccc7)cc6)n5)c4c3n(-c3cc([2H])c([2H])cc3[2H])c2c1 ; [2H]c1cc(-n2c3cc([2H])c([2H])c([2H])c3c3c([2H])c4c5c([2H])c([2H])c([2H])c([2H])c5n(-c5ccc(-c6ccccc6)cc5-c5ccc(-c6nc(-c7ccccc7)nc(-c7ccccc7)n6)cc5)c4cc32)c([2H])c([2H])c1[2H]]

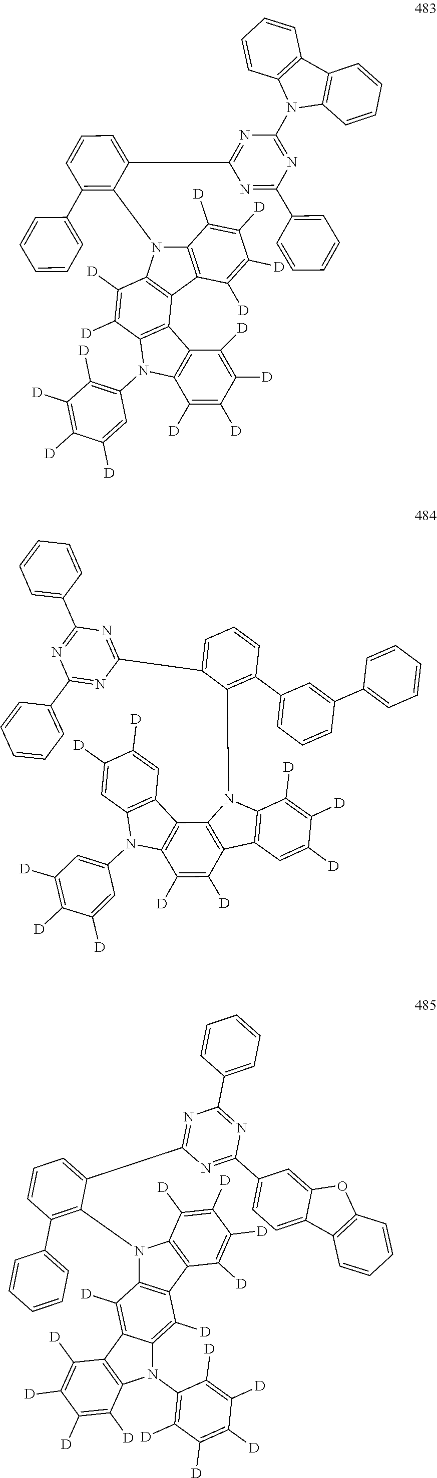

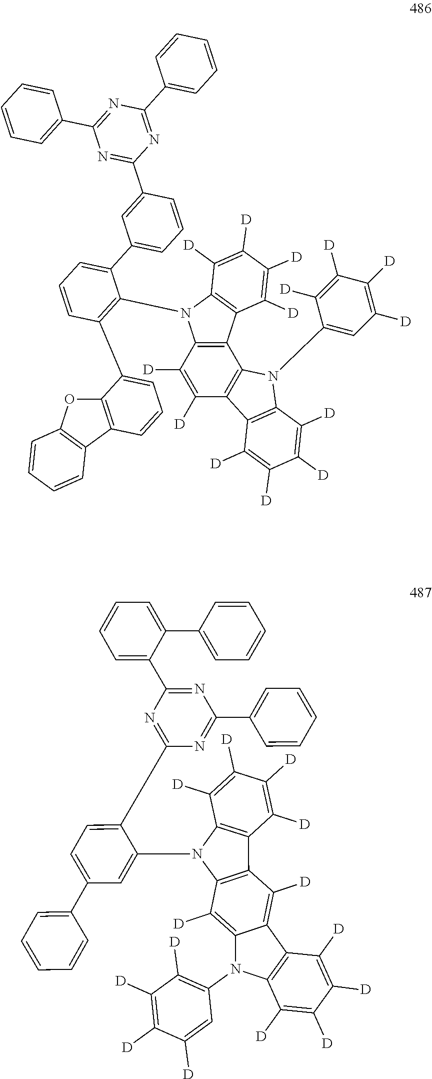 [SMILES: [2H]c1cc(-n2c3c([2H])c([2H])c([2H])c([2H])c3c3c([2H])c([2H])c4c(c5c([2H])c([2H])c([2H])c([2H])c5n4-c4c(-c5cccc(-c6nc(-c7ccccc7)nc(-c7ccccc7)n6)c5)cccc4-c4cccc5c4oc4ccccc45)c32)c([2H])c([2H])c1[2H] ; [2H]c1cc(-n2c3c([2H])c([2H])c([2H])c([2H])c3c3c([2H])c4c5c([2H])c([2H])c([2H])c([2H])c5n(-c5cc(-c6ccccc6)ccc5-c5nc(-c6ccccc6)nc(-c6ccccc6-c6ccccc6)n5)c4c([2H])c32)c([2H])c([2H])c1[2H]]

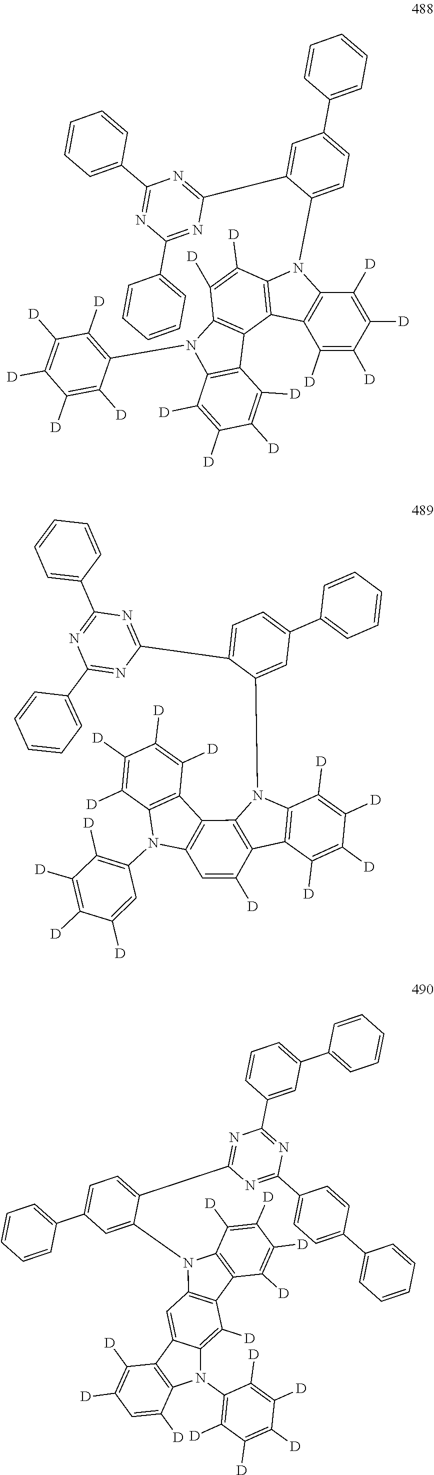

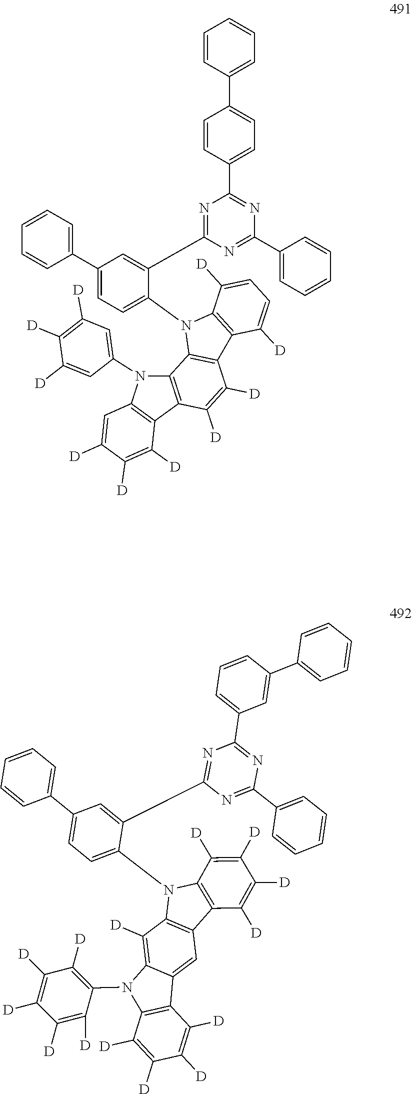 [SMILES: [2H]c1cc(-n2c3cc([2H])c([2H])c([2H])c3c3c([2H])c([2H])c4c5c([2H])ccc([2H])c5n(-c5ccc(-c6ccccc6)cc5-c5nc(-c6ccccc6)nc(-c6ccc(-c7ccccc7)cc6)n5)c4c32)cc([2H])c1[2H] ; [2H]c1c([2H])c([2H])c(-n2c3c([2H])c4c(cc3c3c([2H])c([2H])c([2H])c([2H])c32)c2c([2H])c([2H])c([2H])c([2H])c2n4-c2ccc(-c3ccccc3)cc2-c2nc(-c3ccccc3)nc(-c3cccc(-c4ccccc4)c3)n2)c([2H])c1[2H]]

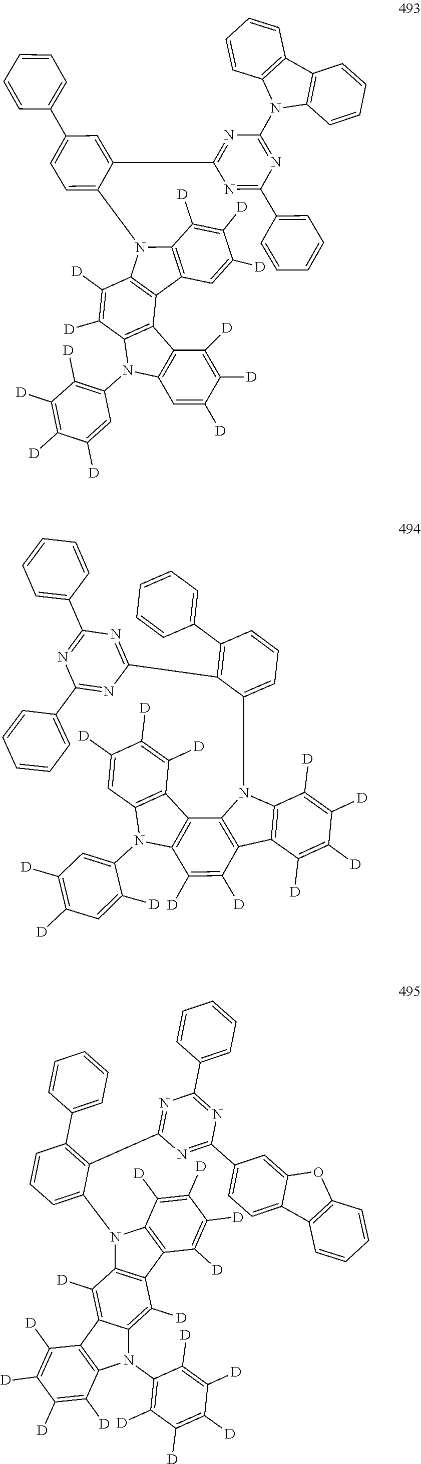

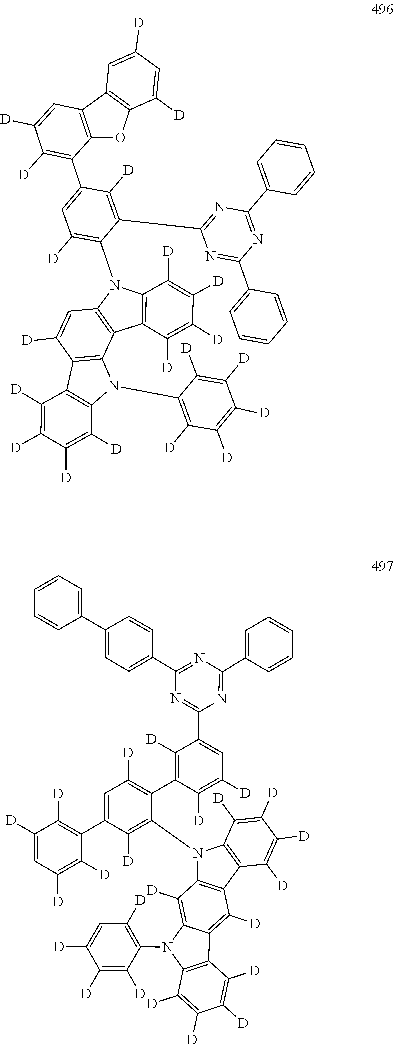 [SMILES: [2H]c1cc([2H])c2oc3c(-c4cc([2H])c(-n5c6cc([2H])c7c8c([2H])c([2H])c([2H])c([2H])c8n(-c8c([2H])c([2H])c([2H])c([2H])c8[2H])c7c6c6c([2H])c([2H])c([2H])c([2H])c65)c(-c5nc(-c6ccccc6)nc(-c6ccccc6)n5)c4[2H])c([2H])c([2H])cc3c2c1 ; [2H]c1cc([2H])c(-n2c3c([2H])c([2H])c([2H])c([2H])c3c3c([2H])c4c5c([2H])c([2H])c([2H])c([2H])c5n(-c5c([2H])c(-c6c([2H])c([2H])cc([2H])c6[2H])cc([2H])c5-c5c([2H])c([2H])cc(-c6nc(-c7ccccc7)nc(-c7ccc(-c8ccccc8)cc7)n6)c5[2H])c4c([2H])c32)c([2H])c1[2H]]

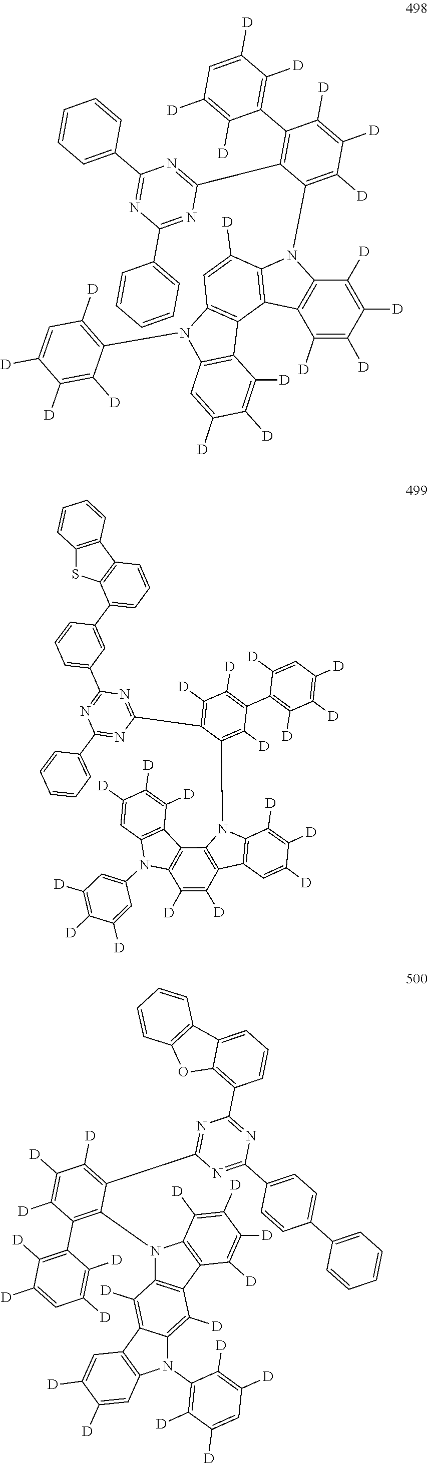

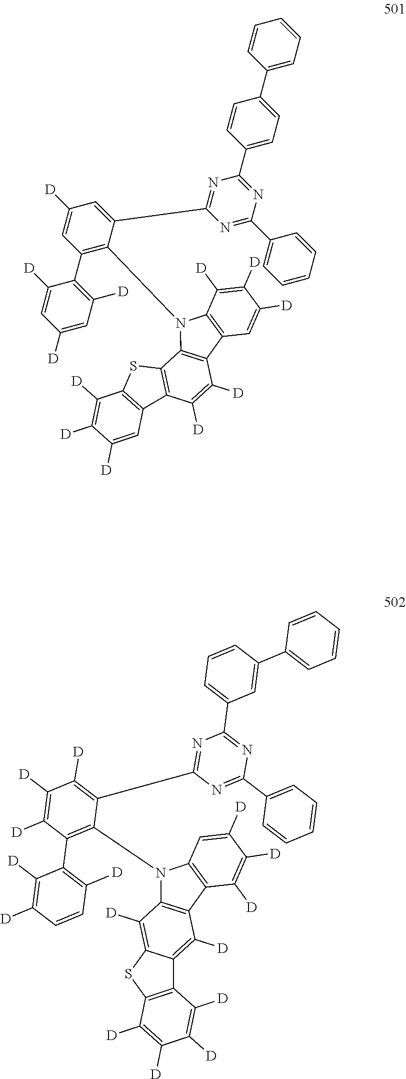 [SMILES: [2H]c1cc([2H])c(-c2cc([2H])cc(-c3nc(-c4ccccc4)nc(-c4ccc(-c5ccccc5)cc4)n3)c2-n2c3c([2H])c([2H])c([2H])cc3c3c([2H])c([2H])c4c5cc([2H])c([2H])c([2H])c5sc4c32)c([2H])c1 ; [2H]c1ccc([2H])c(-c2c([2H])c([2H])c([2H])c(-c3nc(-c4ccccc4)nc(-c4cccc(-c5ccccc5)c4)n3)c2-n2c3cc([2H])c([2H])c([2H])c3c3c([2H])c4c(sc5c([2H])c([2H])c([2H])c([2H])c54)c([2H])c32)c1[2H]]

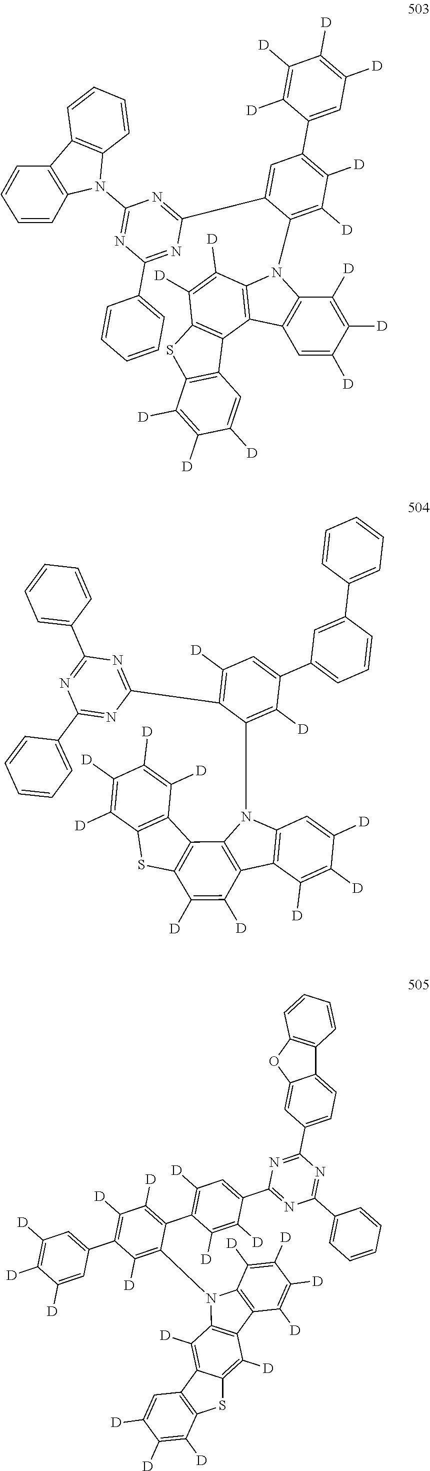

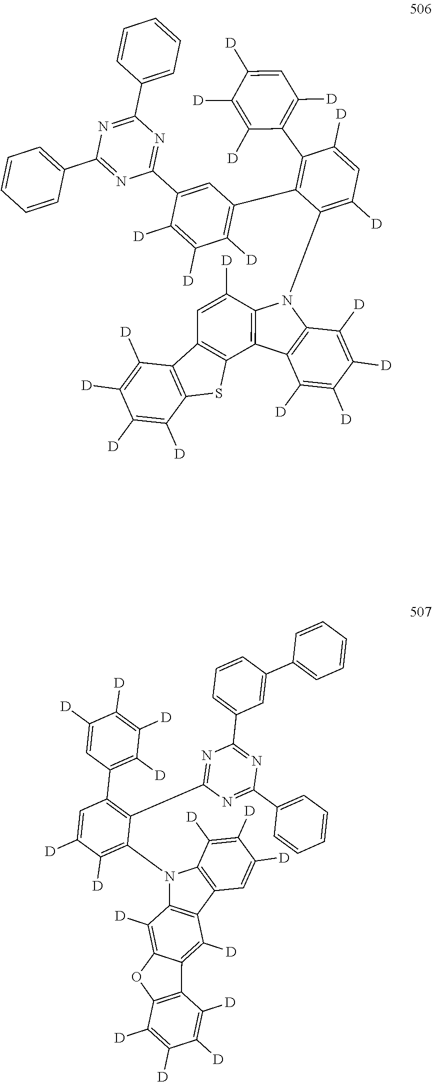 [SMILES: [2H]c1cc([2H])c(-c2c([2H])cc([2H])c(-n3c4c([2H])c([2H])c([2H])c([2H])c4c4c5sc6c([2H])c([2H])c([2H])c([2H])c6c5cc([2H])c43)c2-c2cc(-c3nc(-c4ccccc4)nc(-c4ccccc4)n3)c([2H])c([2H])c2[2H])c([2H])c1[2H] ; [2H]c1cc(-c2cc([2H])c([2H])c(-n3c4c([2H])c([2H])c([2H])cc4c4c([2H])c5c(oc6c([2H])c([2H])c([2H])c([2H])c65)c([2H])c43)c2-c2nc(-c3ccccc3)nc(-c3cccc(-c4ccccc4)c3)n2)c([2H])c([2H])c1[2H]]

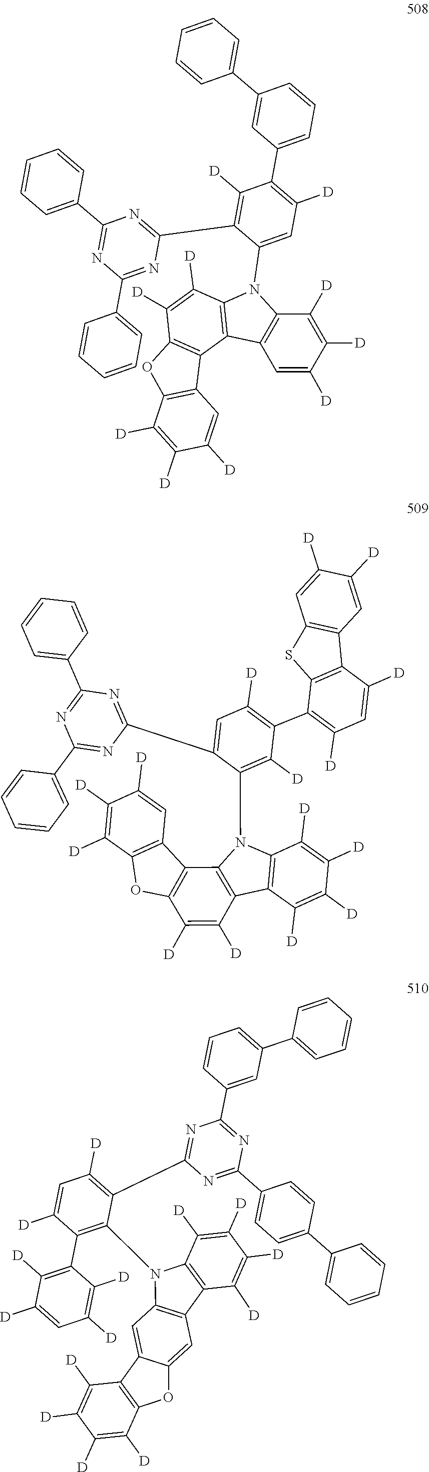

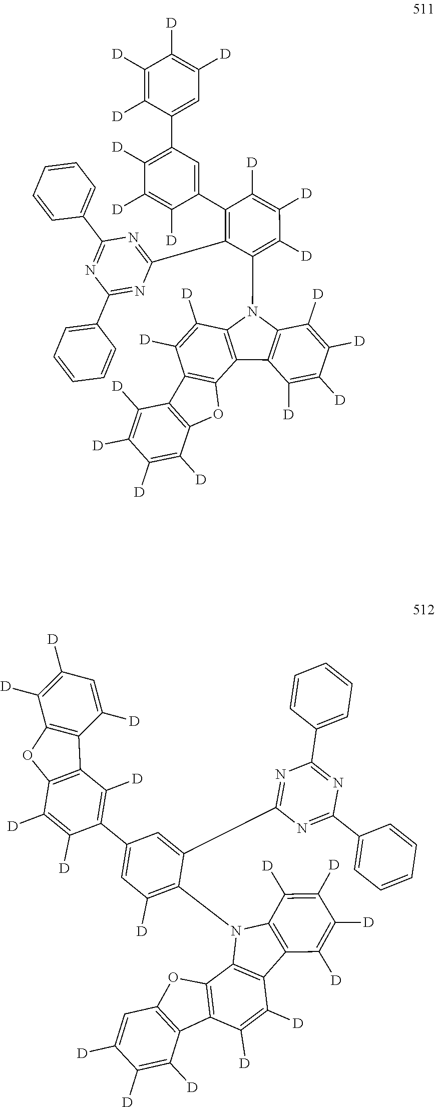 [SMILES: [2H]c1cc(-c2cc(-c3c([2H])c([2H])c([2H])c(-n4c5c([2H])c([2H])c([2H])c([2H])c5c5c6oc7c([2H])c([2H])c([2H])c([2H])c7c6c([2H])c([2H])c54)c3-c3nc(-c4ccccc4)nc(-c4ccccc4)n3)c([2H])c([2H])c2[2H])c([2H])c([2H])c1[2H] ; [2H]c1cc2oc3c(c([2H])c([2H])c4c5c([2H])c([2H])c([2H])c([2H])c5n(-c5c([2H])cc(-c6c([2H])c([2H])c7oc8c([2H])c([2H])cc([2H])c8c7c6[2H])cc5-c5nc(-c6ccccc6)nc(-c6ccccc6)n5)c34)c2c([2H])c1[2H]]

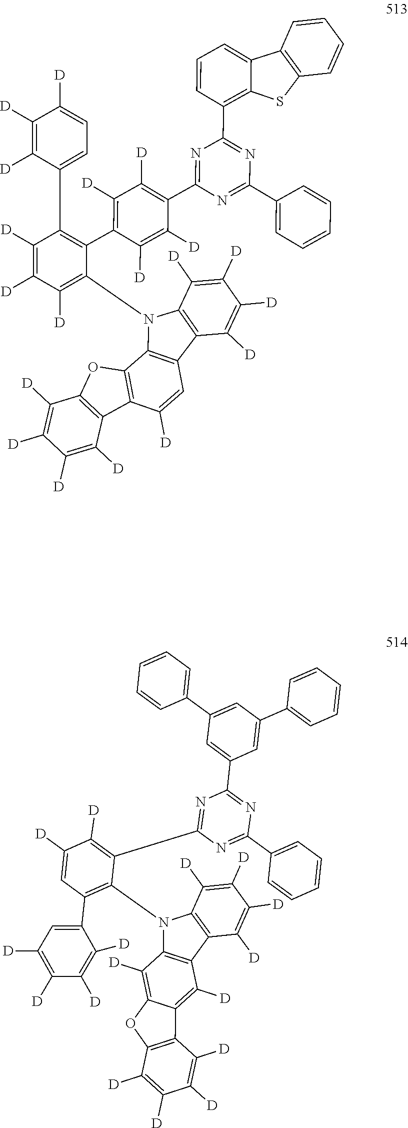 [SMILES: [2H]c1ccc(-c2c([2H])c([2H])c([2H])c(-n3c4c([2H])c([2H])c([2H])c([2H])c4c4cc([2H])c5c(oc6c([2H])c([2H])c([2H])c([2H])c65)c43)c2-c2c([2H])c([2H])c(-c3nc(-c4ccccc4)nc(-c4cccc5c4sc4ccccc45)n3)c([2H])c2[2H])c([2H])c1[2H] ; [2H]c1cc(-c2cc([2H])c([2H])c(-c3nc(-c4ccccc4)nc(-c4cc(-c5ccccc5)cc(-c5ccccc5)c4)n3)c2-n2c3c([2H])c([2H])c([2H])c([2H])c3c3c([2H])c4c(oc5c([2H])c([2H])c([2H])c([2H])c54)c([2H])c32)c([2H])c([2H])c1[2H]]

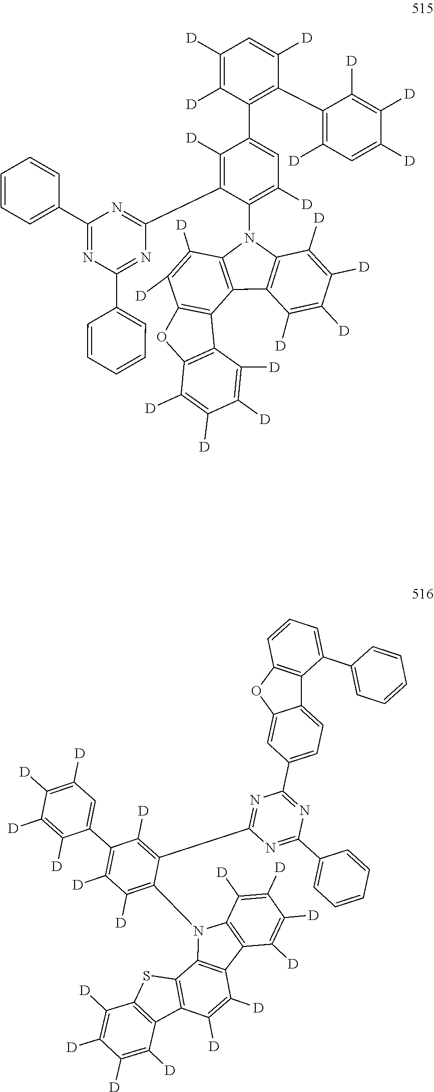 [SMILES: [2H]c1cc([2H])c(-c2c([2H])cc([2H])c([2H])c2-c2cc([2H])c(-n3c4c([2H])c([2H])c([2H])c([2H])c4c4c5c(oc6c([2H])c([2H])c([2H])c([2H])c65)c([2H])c([2H])c43)c(-c3nc(-c4ccccc4)nc(-c4ccccc4)n3)c2[2H])c([2H])c1[2H] ; [2H]c1cc(-c2c([2H])c([2H])c(-n3c4c([2H])c([2H])c([2H])c([2H])c4c4c([2H])c([2H])c5c(sc6c([2H])c([2H])c([2H])c([2H])c65)c43)c(-c3nc(-c4ccccc4)nc(-c4ccc5c(c4)oc4cccc(-c6ccccc6)c45)n3)c2[2H])c([2H])c([2H])c1[2H]]

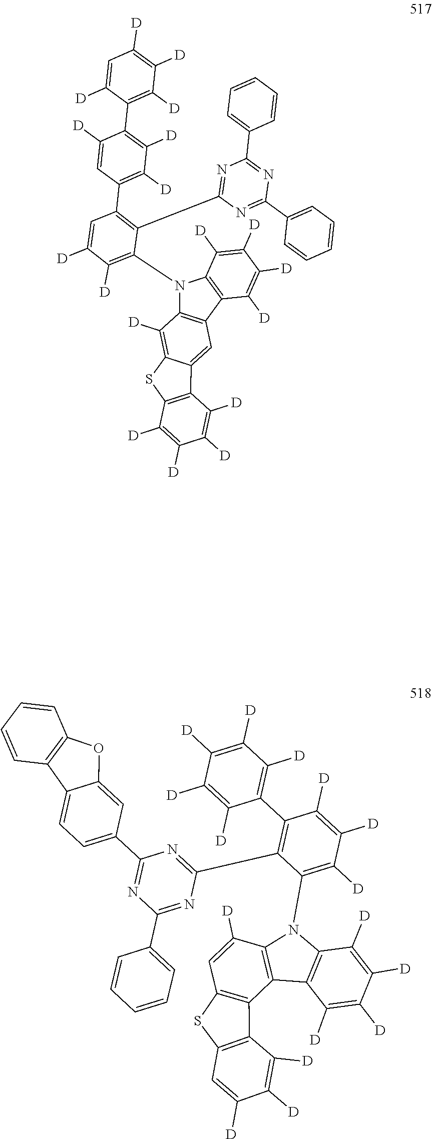 [SMILES: [2H]c1cc([2H])c(-c2c([2H])cc(-c3cc([2H])c([2H])c(-n4c5c([2H])c6sc7c([2H])c([2H])c([2H])c([2H])c7c6cc5c5c([2H])c([2H])c([2H])c([2H])c54)c3-c3nc(-c4ccccc4)nc(-c4ccccc4)n3)c([2H])c2[2H])c([2H])c1[2H] ; [2H]c1cc2sc3cc([2H])c4c(c5c([2H])c([2H])c([2H])c([2H])c5n4-c4c([2H])c([2H])c([2H])c(-c5c([2H])c([2H])c([2H])c([2H])c5[2H])c4-c4nc(-c5ccccc5)nc(-c5ccc6c(c5)oc5ccccc56)n4)c3c2c([2H])c1[2H]]

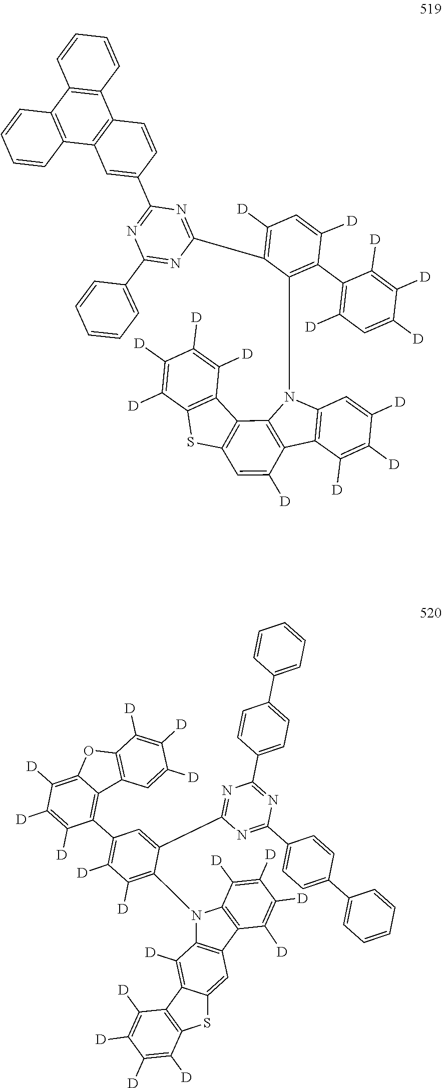 [SMILES: [2H]c1cc([2H])c(-c2c([2H])cc([2H])c(-c3nc(-c4ccccc4)nc(-c4ccc5c6ccccc6c6ccccc6c5c4)n3)c2-n2c3cc([2H])c([2H])c([2H])c3c3c([2H])cc4sc5c([2H])c([2H])c([2H])c([2H])c5c4c32)c([2H])c1[2H] ; [2H]c1cc2c(oc3c([2H])c([2H])c([2H])c(-c4cc(-c5nc(-c6ccc(-c7ccccc7)cc6)nc(-c6ccc(-c7ccccc7)cc6)n5)c(-n5c6c([2H])c7c(cc6c6c([2H])c([2H])c([2H])c([2H])c65)sc5c([2H])c([2H])c([2H])c([2H])c57)c([2H])c4[2H])c32)c([2H])c1[2H]]

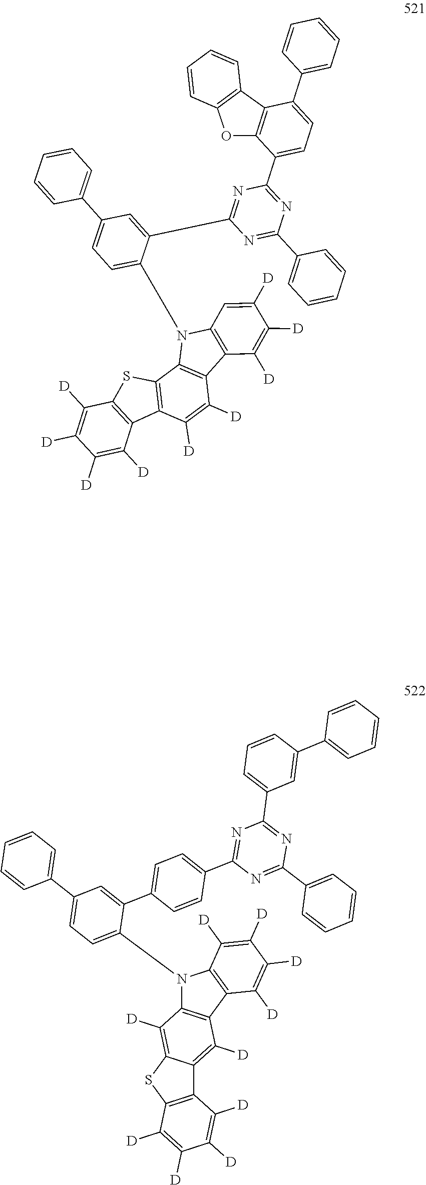 [SMILES: [2H]c1cc2c(c([2H])c1[2H])c1c([2H])c([2H])c3c(sc4c([2H])c([2H])c([2H])c([2H])c43)c1n2-c1ccc(-c2ccccc2)cc1-c1nc(-c2ccccc2)nc(-c2ccc(-c3ccccc3)c3c2oc2ccccc23)n1 ; [2H]c1c([2H])c([2H])c2c(sc3c([2H])c4c(c([2H])c32)c2c([2H])c([2H])c([2H])c([2H])c2n4-c2ccc(-c3ccccc3)cc2-c2ccc(-c3nc(-c4ccccc4)nc(-c4cccc(-c5ccccc5)c4)n3)cc2)c1[2H]]

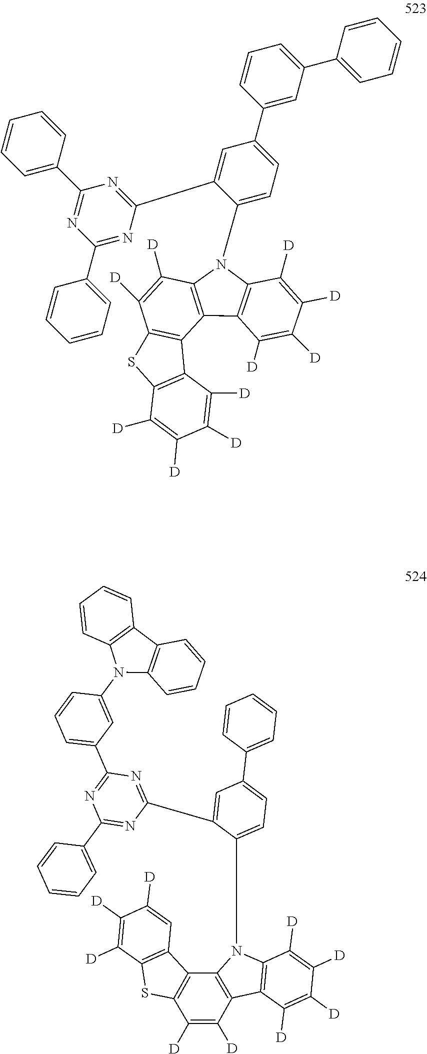 [SMILES: [2H]c1c([2H])c([2H])c2c(sc3c([2H])c([2H])c4c(c5c([2H])c([2H])c([2H])c([2H])c5n4-c4ccc(-c5cccc(-c6ccccc6)c5)cc4-c4nc(-c5ccccc5)nc(-c5ccccc5)n4)c32)c1[2H] ; [2H]c1cc2c(sc3c([2H])c([2H])c4c5c([2H])c([2H])c([2H])c([2H])c5n(-c5ccc(-c6ccccc6)cc5-c5nc(-c6ccccc6)nc(-c6cccc(-n7c8ccccc8c8ccccc87)c6)n5)c4c32)c([2H])c1[2H]]

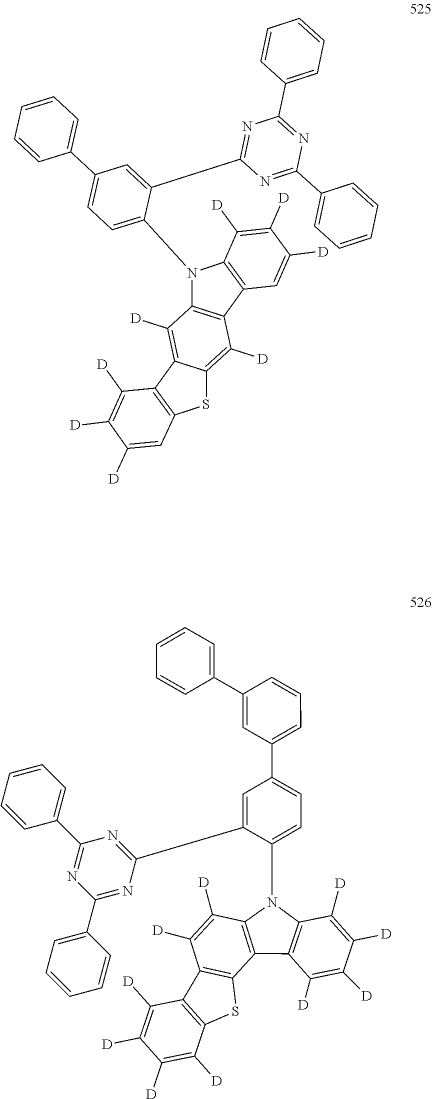 [SMILES: [2H]c1cc2sc3c([2H])c4c5cc([2H])c([2H])c([2H])c5n(-c5ccc(-c6ccccc6)cc5-c5nc(-c6ccccc6)nc(-c6ccccc6)n5)c4c([2H])c3c2c([2H])c1[2H] ; [2H]c1c([2H])c([2H])c2c(sc3c2c([2H])c([2H])c2c3c3c([2H])c([2H])c([2H])c([2H])c3n2-c2ccc(-c3cccc(-c4ccccc4)c3)cc2-c2nc(-c3ccccc3)nc(-c3ccccc3)n2)c1[2H]]

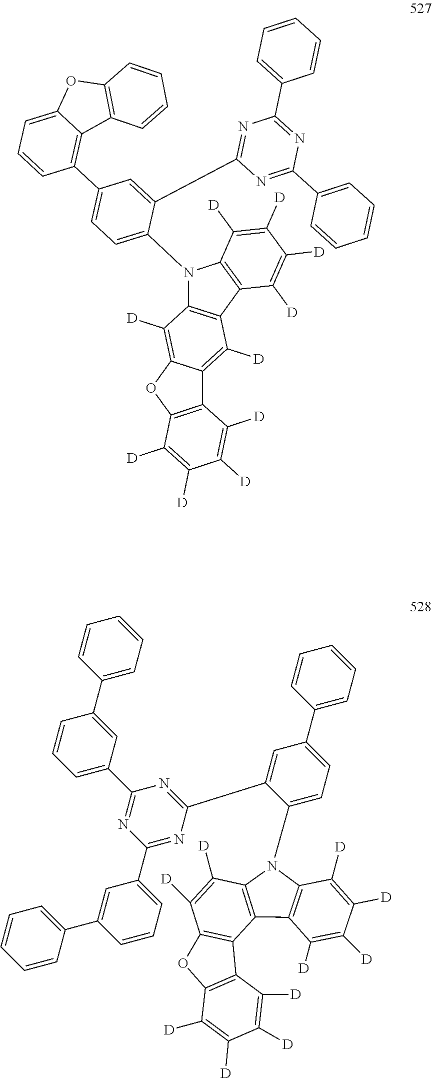 [SMILES: [2H]c1c([2H])c([2H])c2c(oc3c([2H])c4c(c([2H])c32)c2c([2H])c([2H])c([2H])c([2H])c2n4-c2ccc(-c3cccc4oc5ccccc5c34)cc2-c2nc(-c3ccccc3)nc(-c3ccccc3)n2)c1[2H] ; [2H]c1c([2H])c([2H])c2c(oc3c([2H])c([2H])c4c(c5c([2H])c([2H])c([2H])c([2H])c5n4-c4ccc(-c5ccccc5)cc4-c4nc(-c5cccc(-c6ccccc6)c5)nc(-c5cccc(-c6ccccc6)c5)n4)c32)c1[2H]]

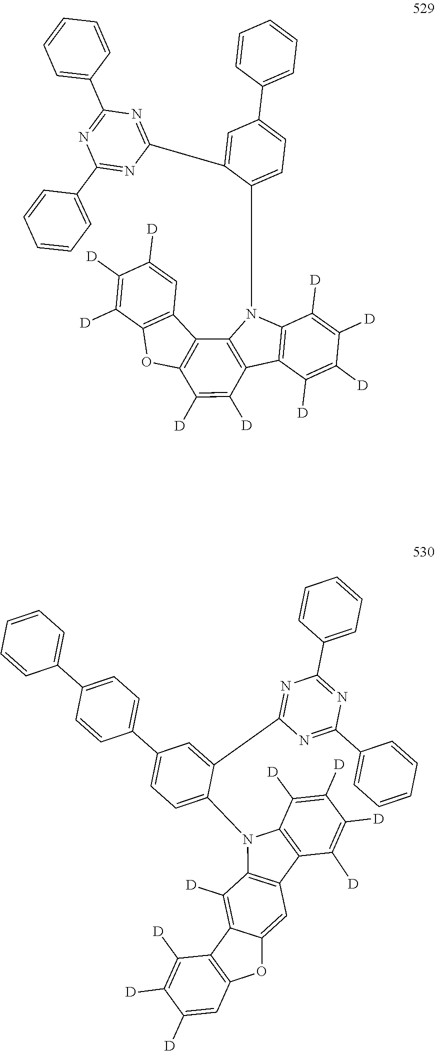 [SMILES: [2H]c1cc2c(oc3c([2H])c([2H])c4c5c([2H])c([2H])c([2H])c([2H])c5n(-c5ccc(-c6ccccc6)cc5-c5nc(-c6ccccc6)nc(-c6ccccc6)n5)c4c32)c([2H])c1[2H] ; [2H]c1cc2oc3cc4c5c([2H])c([2H])c([2H])c([2H])c5n(-c5ccc(-c6ccc(-c7ccccc7)cc6)cc5-c5nc(-c6ccccc6)nc(-c6ccccc6)n5)c4c([2H])c3c2c([2H])c1[2H]]

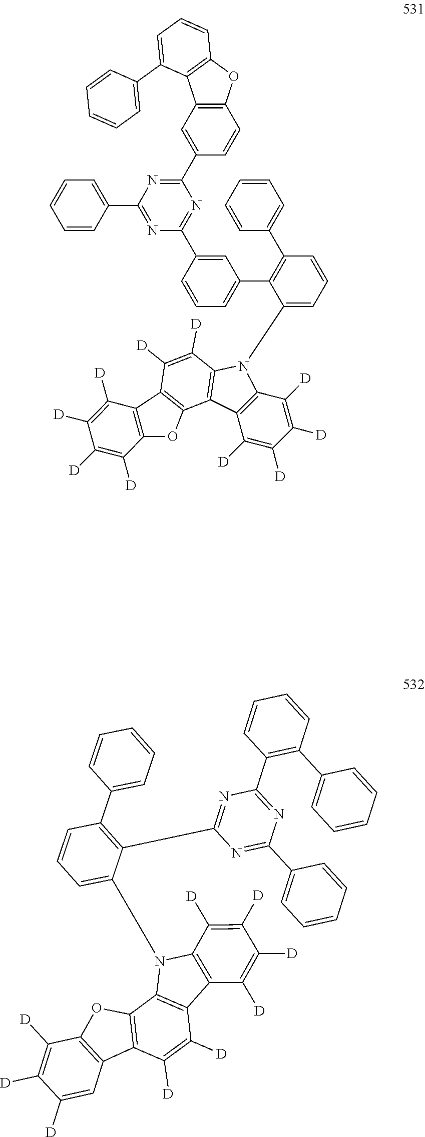 [SMILES: [2H]c1c([2H])c([2H])c2c(oc3c2c([2H])c([2H])c2c3c3c([2H])c([2H])c([2H])c([2H])c3n2-c2cccc(-c3ccccc3)c2-c2cccc(-c3nc(-c4ccccc4)nc(-c4ccc5oc6cccc(-c7ccccc7)c6c5c4)n3)c2)c1[2H] ; [2H]c1cc2c(oc3c2c([2H])c([2H])c2c4c([2H])c([2H])c([2H])c([2H])c4n(-c4cccc(-c5ccccc5)c4-c4nc(-c5ccccc5)nc(-c5ccccc5-c5ccccc5)n4)c32)c([2H])c1[2H]]

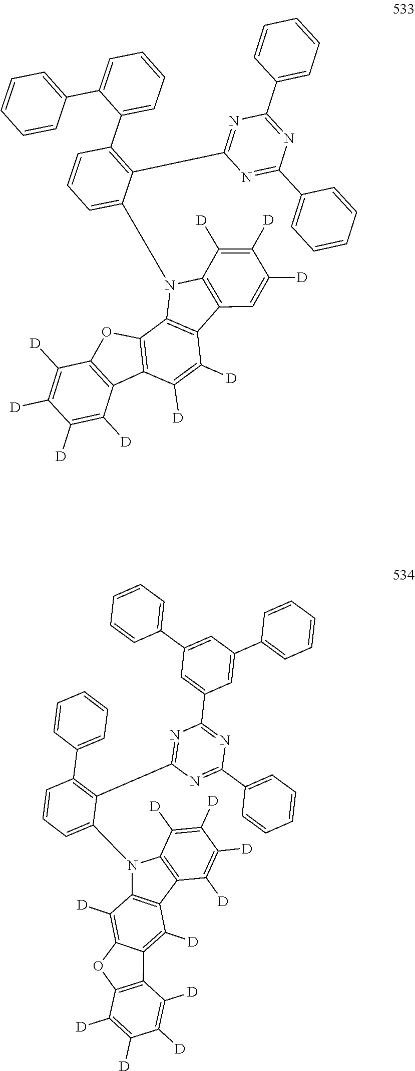 [SMILES: [2H]c1cc2c3c([2H])c([2H])c4c(oc5c([2H])c([2H])c([2H])c([2H])c54)c3n(-c3cccc(-c4ccccc4-c4ccccc4)c3-c3nc(-c4ccccc4)nc(-c4ccccc4)n3)c2c([2H])c1[2H] ; [2H]c1c([2H])c([2H])c2c(oc3c([2H])c4c(c([2H])c32)c2c([2H])c([2H])c([2H])c([2H])c2n4-c2cccc(-c3ccccc3)c2-c2nc(-c3ccccc3)nc(-c3cc(-c4ccccc4)cc(-c4ccccc4)c3)n2)c1[2H]]

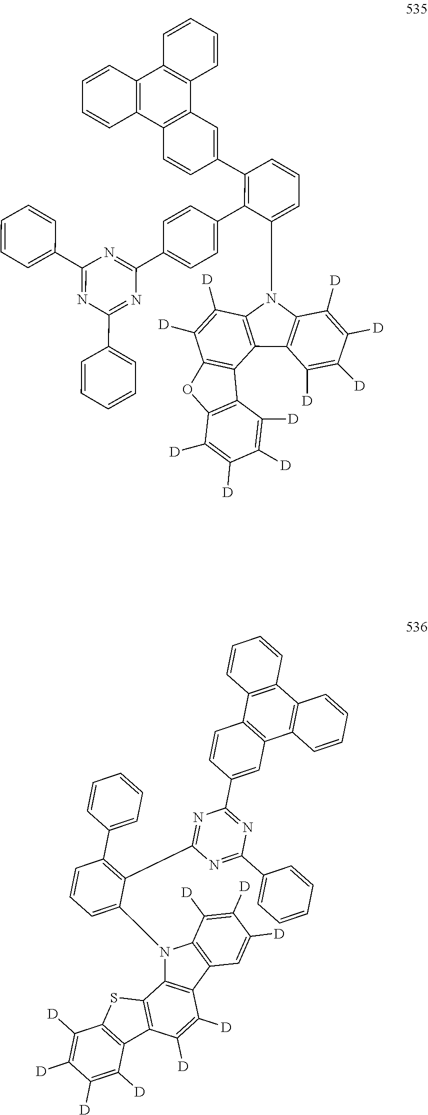 [SMILES: [2H]c1c([2H])c([2H])c2c(oc3c([2H])c([2H])c4c(c5c([2H])c([2H])c([2H])c([2H])c5n4-c4cccc(-c5ccc6c7ccccc7c7ccccc7c6c5)c4-c4ccc(-c5nc(-c6ccccc6)nc(-c6ccccc6)n5)cc4)c32)c1[2H] ; [2H]c1cc2c3c([2H])c([2H])c4c(sc5c([2H])c([2H])c([2H])c([2H])c54)c3n(-c3cccc(-c4ccccc4)c3-c3nc(-c4ccccc4)nc(-c4ccc5c6ccccc6c6ccccc6c5c4)n3)c2c([2H])c1[2H]]

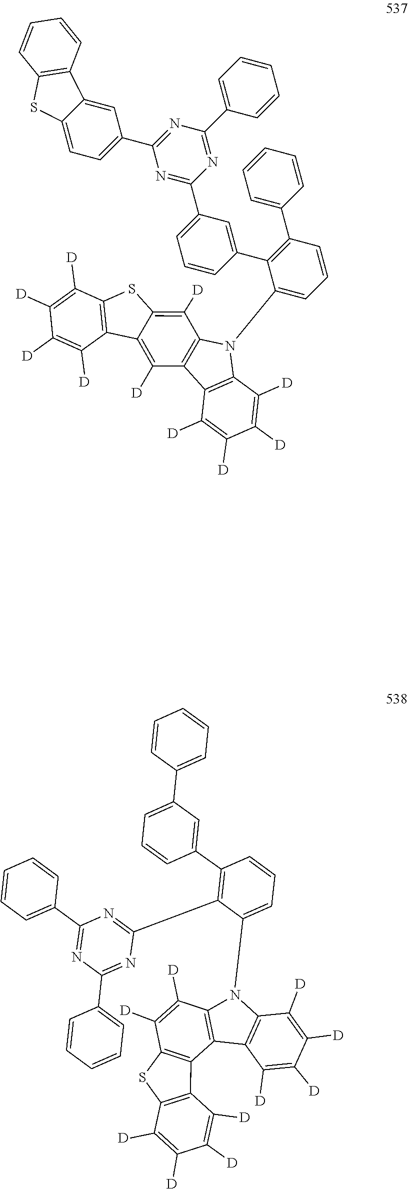 [SMILES: [2H]c1c([2H])c([2H])c2c(sc3c([2H])c4c(c([2H])c32)c2c([2H])c([2H])c([2H])c([2H])c2n4-c2cccc(-c3ccccc3)c2-c2cccc(-c3nc(-c4ccccc4)nc(-c4ccc5sc6ccccc6c5c4)n3)c2)c1[2H] ; [2H]c1c([2H])c([2H])c2c(sc3c([2H])c([2H])c4c(c5c([2H])c([2H])c([2H])c([2H])c5n4-c4cccc(-c5cccc(-c6ccccc6)c5)c4-c4nc(-c5ccccc5)nc(-c5ccccc5)n4)c32)c1[2H]]

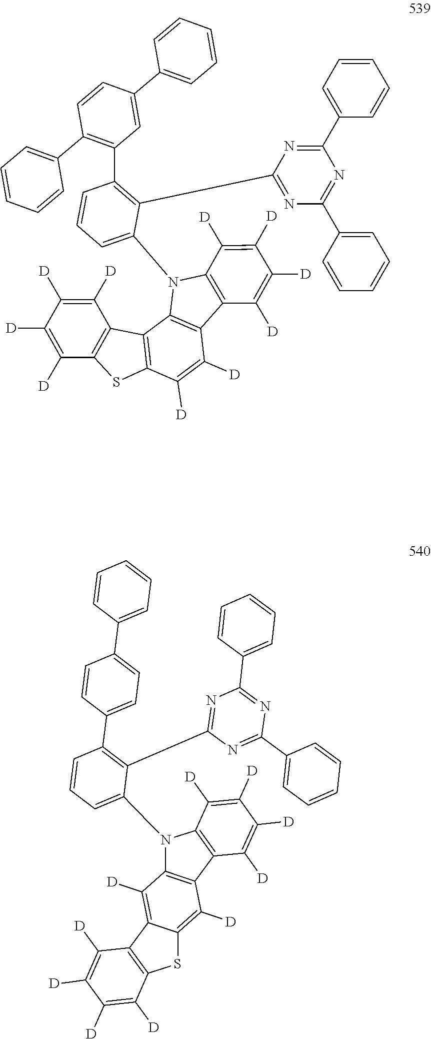 [SMILES: [2H]c1c([2H])c([2H])c2c(sc3c([2H])c([2H])c4c5c([2H])c([2H])c([2H])c([2H])c5n(-c5cccc(-c6cc(-c7ccccc7)ccc6-c6ccccc6)c5-c5nc(-c6ccccc6)nc(-c6ccccc6)n5)c4c32)c1[2H] ; [2H]c1c([2H])c([2H])c2c(sc3c([2H])c4c5c([2H])c([2H])c([2H])c([2H])c5n(-c5cccc(-c6ccc(-c7ccccc7)cc6)c5-c5nc(-c6ccccc6)nc(-c6ccccc6)n5)c4c([2H])c32)c1[2H]]

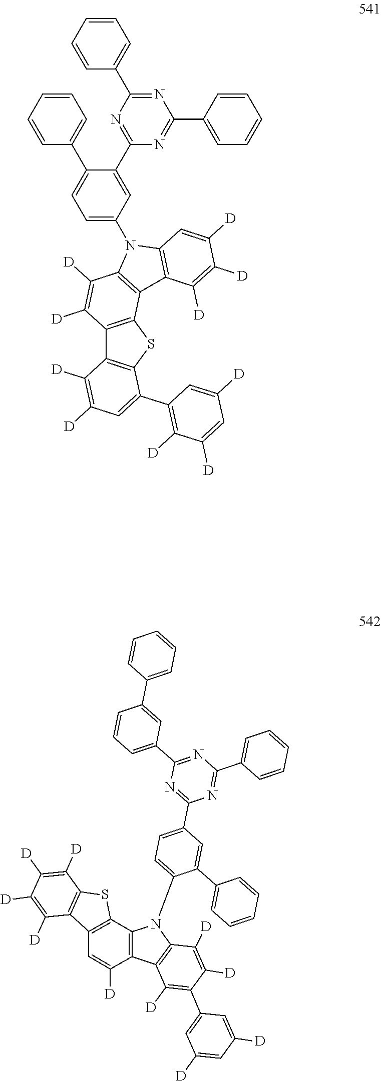 [SMILES: [2H]c1cc([2H])c([2H])c(-c2cc([2H])c([2H])c3c2sc2c3c([2H])c([2H])c3c2c2c([2H])c([2H])c([2H])cc2n3-c2ccc(-c3ccccc3)c(-c3nc(-c4ccccc4)nc(-c4ccccc4)n3)c2)c1 ; [2H]c1cc([2H])cc(-c2c([2H])c([2H])c3c(c2[2H])c2c([2H])cc4c(sc5c([2H])c([2H])c([2H])c([2H])c54)c2n3-c2ccc(-c3nc(-c4ccccc4)nc(-c4cccc(-c5ccccc5)c4)n3)cc2-c2ccccc2)c1]

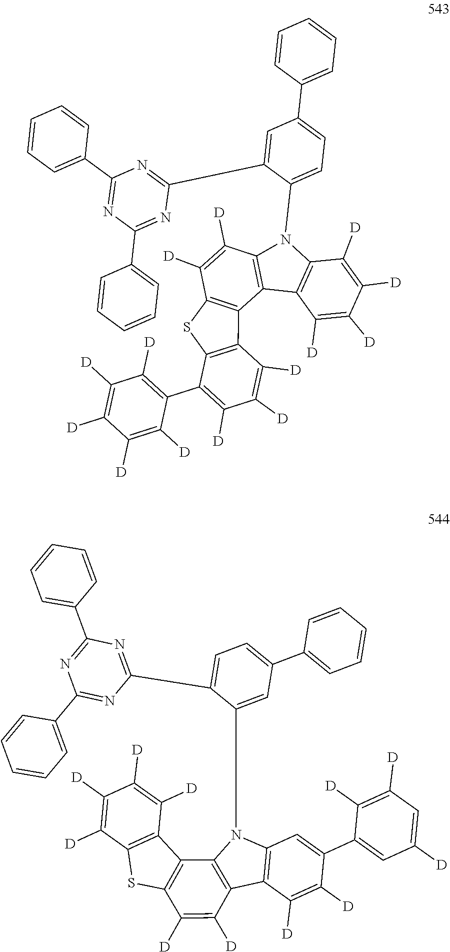 [SMILES: [2H]c1c([2H])c([2H])c(-c2c([2H])c([2H])c([2H])c3c2sc2c([2H])c([2H])c4c(c5c([2H])c([2H])c([2H])c([2H])c5n4-c4ccc(-c5ccccc5)cc4-c4nc(-c5ccccc5)nc(-c5ccccc5)n4)c23)c([2H])c1[2H] ; [2H]c1cc([2H])c([2H])c(-c2cc3c(c([2H])c2[2H])c2c([2H])c([2H])c4sc5c([2H])c([2H])c([2H])c([2H])c5c4c2n3-c2cc(-c3ccccc3)ccc2-c2nc(-c3ccccc3)nc(-c3ccccc3)n2)c1]

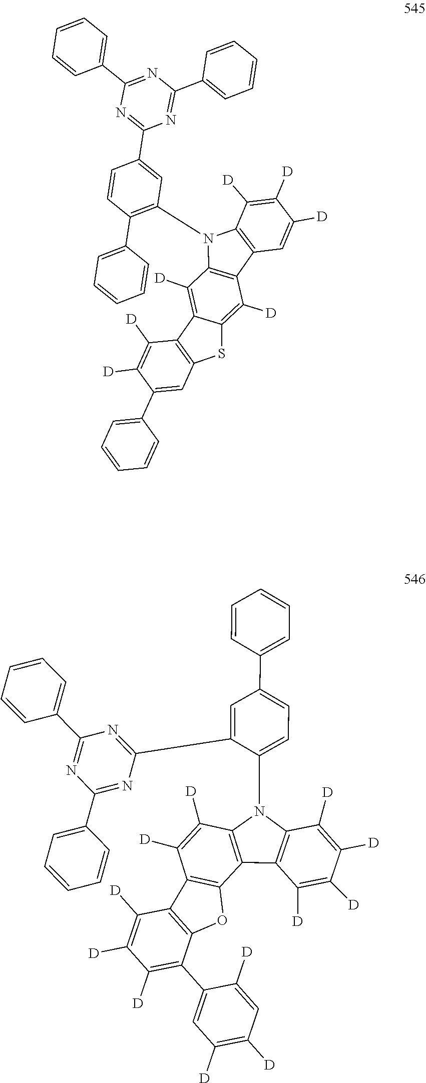 [SMILES: [2H]c1cc2c3c([2H])c4sc5cc(-c6ccccc6)c([2H])c([2H])c5c4c([2H])c3n(-c3cc(-c4nc(-c5ccccc5)nc(-c5ccccc5)n4)ccc3-c3ccccc3)c2c([2H])c1[2H] ; [2H]c1cc([2H])c(-c2c([2H])c([2H])c([2H])c3c2oc2c3c([2H])c([2H])c3c2c2c([2H])c([2H])c([2H])c([2H])c2n3-c2ccc(-c3ccccc3)cc2-c2nc(-c3ccccc3)nc(-c3ccccc3)n2)cc1[2H]]

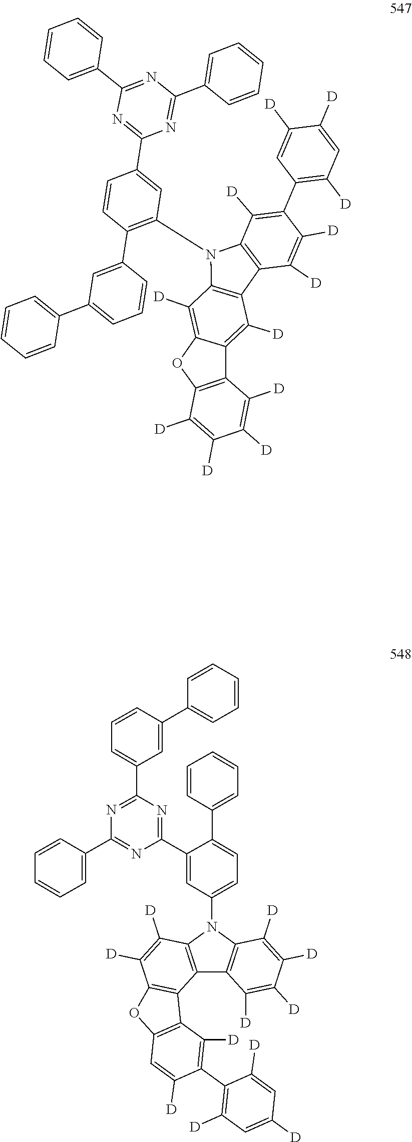 [SMILES: [2H]c1cc([2H])c(-c2c([2H])c([2H])c3c4c([2H])c5c(oc6c([2H])c([2H])c([2H])c([2H])c65)c([2H])c4n(-c4cc(-c5nc(-c6ccccc6)nc(-c6ccccc6)n5)ccc4-c4cccc(-c5ccccc5)c4)c3c2[2H])cc1[2H] ; [2H]c1cc([2H])c(-c2c([2H])cc3oc4c([2H])c([2H])c5c(c6c([2H])c([2H])c([2H])c([2H])c6n5-c5ccc(-c6ccccc6)c(-c6nc(-c7ccccc7)nc(-c7cccc(-c8ccccc8)c7)n6)c5)c4c3c2[2H])c([2H])c1]

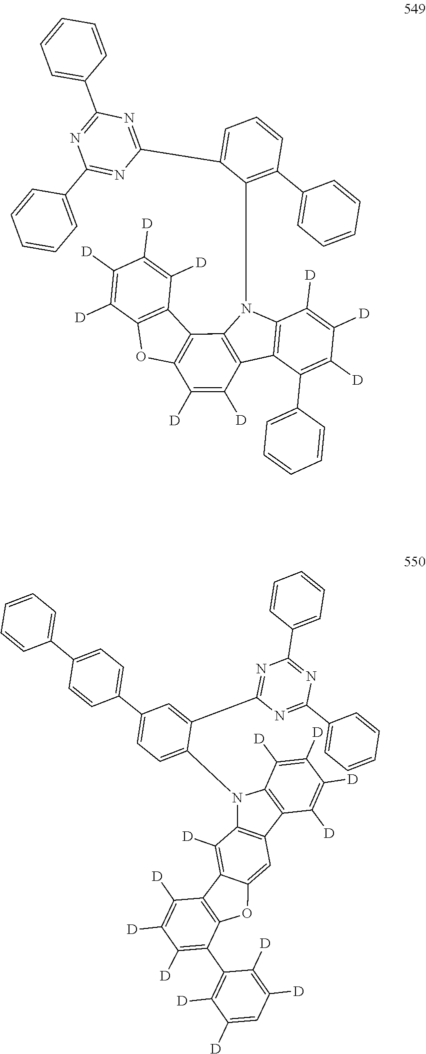 [SMILES: [2H]c1c([2H])c([2H])c2c(oc3c([2H])c([2H])c4c5c(-c6ccccc6)c([2H])c([2H])c([2H])c5n(-c5c(-c6ccccc6)cccc5-c5nc(-c6ccccc6)nc(-c6ccccc6)n5)c4c32)c1[2H] ; [2H]c1cc([2H])c([2H])c(-c2c([2H])c([2H])c([2H])c3c2oc2cc4c5c([2H])c([2H])c([2H])c([2H])c5n(-c5ccc(-c6ccc(-c7ccccc7)cc6)cc5-c5nc(-c6ccccc6)nc(-c6ccccc6)n5)c4c([2H])c23)c1[2H]]

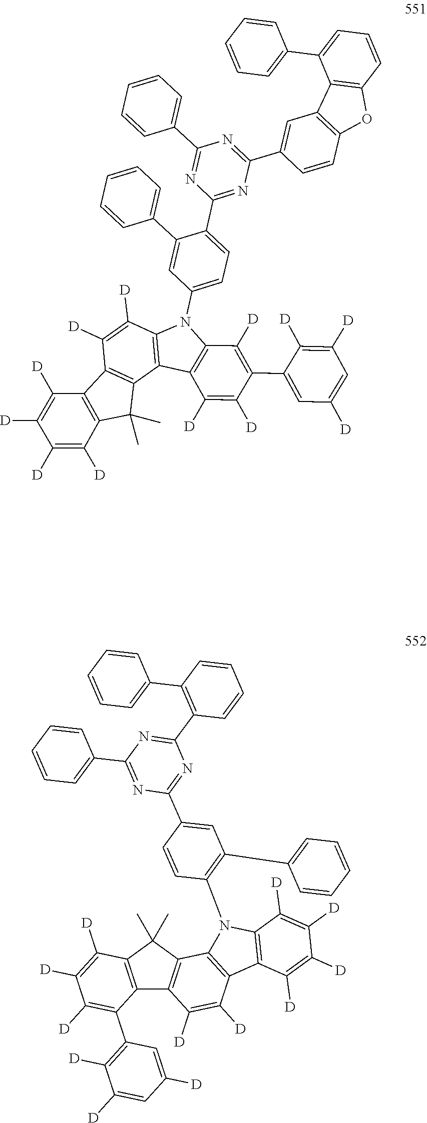 [SMILES: [2H]c1cc([2H])c([2H])c(-c2c([2H])c([2H])c3c4c5c(c([2H])c([2H])c4n(-c4ccc(-c6nc(-c7ccccc7)nc(-c7ccc8oc9cccc(-c%10ccccc%10)c9c8c7)n6)c(-c6ccccc6)c4)c3c2[2H])-c2c([2H])c([2H])c([2H])c([2H])c2C5(C)C)c1 ; [2H]c1cc([2H])c([2H])c(-c2c([2H])c([2H])c([2H])c3c2-c2c([2H])c([2H])c4c5c([2H])c([2H])c([2H])c([2H])c5n(-c5ccc(-c6nc(-c7ccccc7)nc(-c7ccccc7-c7ccccc7)n6)cc5-c5ccccc5)c4c2C3(C)C)c1]

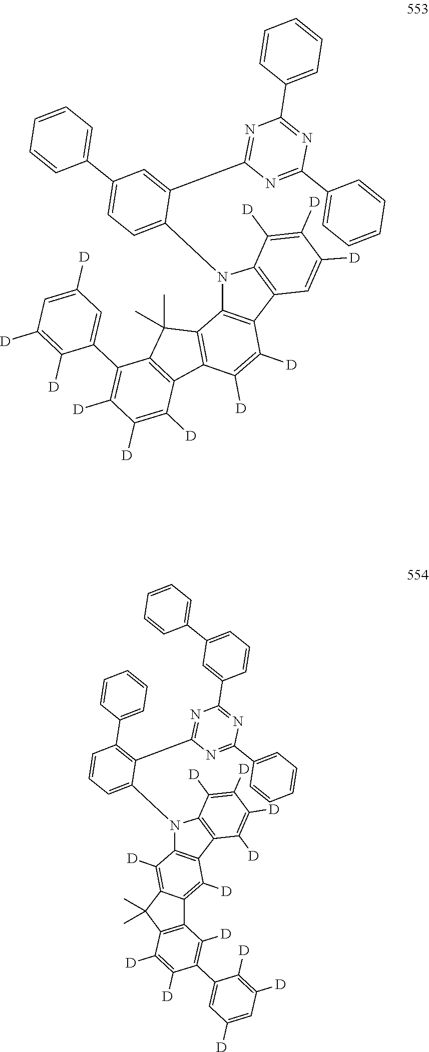 [SMILES: [2H]c1cc([2H])c([2H])c(-c2c([2H])c([2H])c([2H])c3c2C(C)(C)c2c-3c([2H])c([2H])c3c4cc([2H])c([2H])c([2H])c4n(-c4ccc(-c5ccccc5)cc4-c4nc(-c5ccccc5)nc(-c5ccccc5)n4)c23)c1 ; [2H]c1cc([2H])c([2H])c(-c2c([2H])c([2H])c3c(c2[2H])-c2c(c([2H])c4c(c2[2H])c2c([2H])c([2H])c([2H])c([2H])c2n4-c2cccc(-c4ccccc4)c2-c2nc(-c4ccccc4)nc(-c4cccc(-c5ccccc5)c4)n2)C3(C)C)c1]

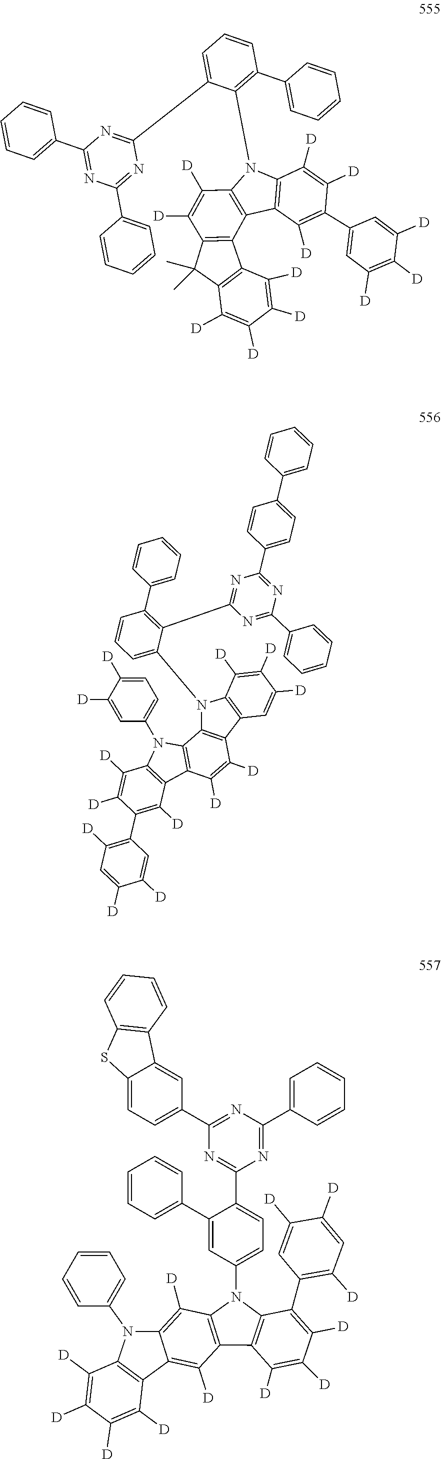

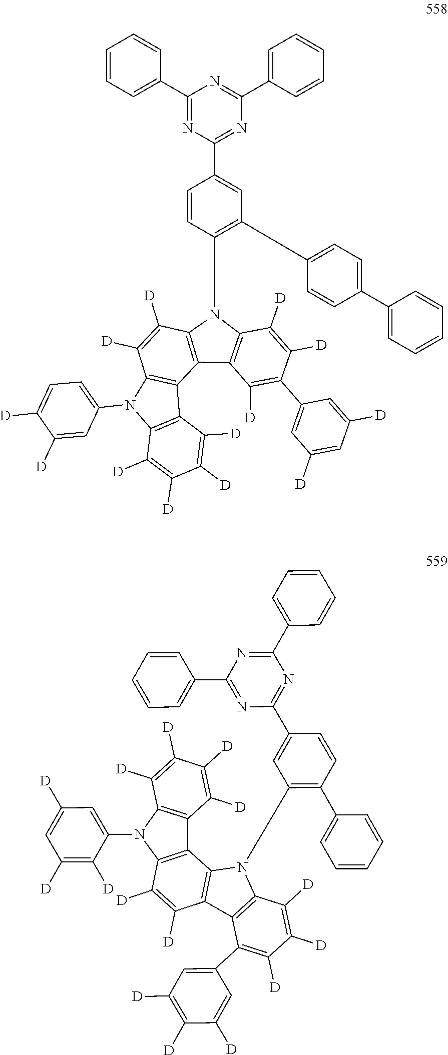 [SMILES: [2H]c1cc([2H])cc(-c2c([2H])c([2H])c3c(c2[2H])c2c4c5c([2H])c([2H])c([2H])c([2H])c5n(-c5ccc([2H])c([2H])c5)c4c([2H])c([2H])c2n3-c2ccc(-c3nc(-c4ccccc4)nc(-c4ccccc4)n3)cc2-c2ccc(-c3ccccc3)cc2)c1 ; [2H]c1cc([2H])c([2H])c(-n2c3c([2H])c([2H])c([2H])c([2H])c3c3c2c([2H])c([2H])c2c4c(-c5cc([2H])c([2H])c([2H])c5)c([2H])c([2H])c([2H])c4n(-c4cc(-c5nc(-c6ccccc6)nc(-c6ccccc6)n5)ccc4-c4ccccc4)c23)c1]

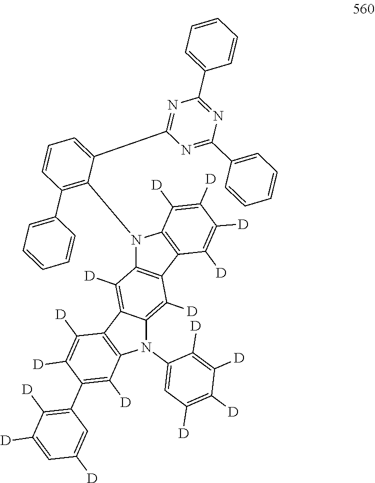 [SMILES: [2H]c1cc([2H])c([2H])c(-c2c([2H])c([2H])c3c4c([2H])c5c(c([2H])c4n(-c4cc([2H])c([2H])c([2H])c4[2H])c3c2[2H])c2c([2H])c([2H])c([2H])c([2H])c2n5-c2c(-c3ccccc3)cccc2-c2nc(-c3ccccc3)nc(-c3ccccc3)n2)c1]

In the compound structure, the deuterium position indicates an arbitrary position, and the substitution position is not specified as long as it satisfies the deuterium substitution rate in a structure where deuterium is substituted.

Further, various substituents may be introduced into the structure of Chemical Formula 1 to synthesize a compound having inherent characteristics of a substituent introduced. For example, it is possible to synthesize a material which satisfies conditions required for each organic material layer by introducing a substituent usually used for a hole injection layer material, a hole transport layer material, a light emitting layer material, an electron transport layer material, and a charge generation layer material, which are used for preparing an organic light emitting device, into the core structure.

In addition, it is possible to finely adjust an energy band-gap by introducing various substituents into the structure of Chemical Formula 1, and meanwhile, it is possible to improve characteristics at the interface between organic materials and diversify the use of the material.

In another exemplary embodiment of the present specification, provided is an organic light emitting device including: a first electrode; a second electrode; and an organic material layer having one or more layers provided between the first electrode and the second electrode, in which one or more layers of the organic material layer include one or more heterocyclic compounds of Chemical Formula 1.

In an exemplary embodiment of the present specification, the organic material layer includes a light emitting layer, and the light emitting layer may include one or more of the heterocyclic compounds.

In an exemplary embodiment of the present specification, the organic material layer includes a light emitting layer, and the light emitting layer may include one of the heterocyclic compounds.

In an exemplary embodiment of the present specification, the organic material layer includes a light emitting layer, and when the light emitting layer includes a host, the host may include one or more of the heterocyclic compounds.

In an exemplary embodiment of the present specification, the organic material layer includes a light emitting layer, the light emitting layer includes a host, and the host may include one of the heterocyclic compounds.

In an exemplary embodiment of the present specification, the organic material layer includes a light emitting layer, the light emitting layer includes a host, the host includes a green host, and the green host may include one or more of the heterocyclic compounds.

In an exemplary embodiment of the present specification, the organic material layer includes a light emitting layer, the light emitting layer includes a host, the host includes a red host, and the red host may include one or more of the heterocyclic compounds.

In an exemplary embodiment of the present specification, the organic material layer includes a light emitting layer, the light emitting layer includes a host, the host includes a blue host, and the blue host may include one or more of the heterocyclic compounds.

In an exemplary embodiment of the present specification, the organic material layer includes a light emitting layer, and the light emitting layer may include the heterocyclic compound as an N-type host.

In an exemplary embodiment of the present specification, the organic material layer includes a light emitting layer, and the light emitting layer may further include a compound of the following Chemical Formula 2 or 3 in addition to the heterocyclic compound.

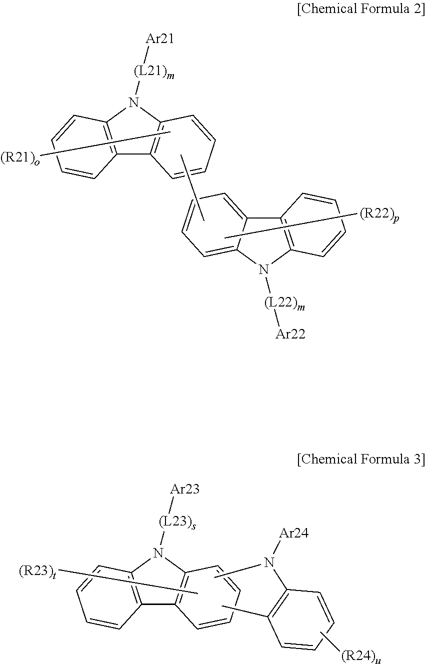

In Chemical Formulae 2 and 3,
L21 to L23 are each independently a direct bond; a substituted or unsubstituted C6 to C60 arylene group; or a substituted or unsubstituted C2 to C60 heteroarylene group,
m, n, and s are each an integer from 1 to 3, and when m, n, and s are each 2 or greater, substituents in the parenthesis are the same as or different from each other,
Ar21 to Ar24 are each independently a substituted or unsubstituted C6 to C60 aryl group; or a substituted or unsubstituted C2 to C60 heteroaryl group,
R21 to R24 are each independently hydrogen; deuterium; a halogen group; a cyano group; a substituted or unsubstituted C1 to C60 alkyl group; a substituted or unsubstituted C6 to C60 aryl group; or a substituted or unsubstituted C2 to C60 heteroaryl group, and
and p are each an integer from 1 to 7, t is an integer from 1 to 6, u is an integer from 1 to 4, and when o, p, t, and u are each 2 or greater, substituents in the parenthesis are the same as or different from each other.

In an exemplary embodiment of the present specification, L21 and L22 may be each independently a direct bond; a substituted or unsubstituted C6 to C30 arylene group; or a substituted or unsubstituted C2 to C30 heteroarylene group.

In an exemplary embodiment of the present specification, L21 and L22 may be each independently a direct bond; a substituted or unsubstituted C6 to C30 arylene group; or a C2 to C30 heteroarylene group which is substituted or unsubstituted and includes O.

In an exemplary embodiment of the present specification, L21 and L22 may be each independently a direct bond; a substituted or unsubstituted phenylene group; or a substituted or unsubstituted divalent dibenzofuran group.

In an exemplary embodiment of the present specification, L21 and L22 may be each independently a direct bond; a C6 to C30 arylene group unsubstituted or substituted with deuterium; or a C2 to C30 heteroarylene group unsubstituted or substituted with deuterium.

In an exemplary embodiment of the present specification, L21 and L22 may be each independently a direct bond; or a substituted or unsubstituted C6 to C30 arylene group.

In an exemplary embodiment of the present specification, L21 and L22 may be each independently a direct bond; or a substituted or unsubstituted phenylene group.

In an exemplary embodiment of the present specification, L21 and L22 may be each independently a direct bond; or a C6 to C30 arylene group unsubstituted or substituted with deuterium.

In an exemplary embodiment of the present specification, Ar21 and Ar22 may be each independently a substituted or unsubstituted C6 to C30 aryl group; or a substituted or unsubstituted C2 to C30 heteroaryl group.

In an exemplary embodiment of the present specification, Ar21 and Ar22 may be each independently a substituted or unsubstituted C6 to C30 aryl group; or a C2 to C30 heteroaryl group which is substituted or unsubstituted and includes O or S.

In an exemplary embodiment of the present specification, Ar21 and Ar22 may be each independently a substituted or unsubstituted phenyl group; a substituted or unsubstituted biphenyl group; a substituted or unsubstituted terphenyl group; a substituted or unsubstituted triphenylene group; a substituted or unsubstituted dibenzofuran group; or a substituted or unsubstituted dibenzothiophene group.

In an exemplary embodiment of the present specification, Ar21 and Ar22 may be each independently a C6 to C30 aryl group unsubstituted or substituted with deuterium; or a C2 to C30 heteroaryl group which is unsubstituted or substituted with deuterium and includes O or S.

In an exemplary embodiment of the present specification, Ar21 and Ar22 may be each independently a phenyl group unsubstituted or substituted with deuterium; a biphenyl group unsubstituted or substituted with deuterium; a terphenyl group unsubstituted or substituted with deuterium; a triphenylene group unsubstituted or substituted with deuterium; a dibenzofuran group unsubstituted or substituted with deuterium; or a dibenzothiophene group unsubstituted or substituted with deuterium.

In an exemplary embodiment of the present specification, R21 and R22 may be each independently hydrogen; deuterium; a substituted or unsubstituted C1 to C60 alkyl group; a substituted or unsubstituted C6 to C60 aryl group; or a substituted or unsubstituted C2 to C60 heteroaryl group.

In an exemplary embodiment of the present specification, R21 and R22 may be each independently hydrogen; deuterium; a substituted or unsubstituted C1 to C10 alkyl group; a substituted or unsubstituted C6 to C20 aryl group; or a substituted or unsubstituted C2 to C20 heteroaryl group.

In an exemplary embodiment of the present specification, R21 and R22 may be each independently hydrogen; or deuterium.

In an exemplary embodiment of the present specification, Ar23 and Ar24 may be each independently a substituted or unsubstituted C6 to C30 aryl group; or a substituted or unsubstituted C2 to C30 heteroaryl group.

In an exemplary embodiment of the present specification, Ar23 and Ar24 may be each independently a substituted or unsubstituted C6 to C30 aryl group; or a C2 to C30 heteroaryl group which is substituted or unsubstituted and includes O.

In an exemplary embodiment of the present specification, Ar23 and Ar24 may be each independently a substituted or unsubstituted phenyl group; a substituted or unsubstituted biphenyl group; a substituted or unsubstituted terphenyl group; a substituted or unsubstituted triphenylene group; or a substituted or unsubstituted dibenzofuran group.

In an exemplary embodiment of the present specification, Ar23 and Ar24 may be each independently a C6 to C30 aryl group unsubstituted or substituted with deuterium; or a C2 to C30 heteroaryl group which is unsubstituted or substituted with deuterium and includes O.

In an exemplary embodiment of the present specification, Ar23 and Ar24 may be each independently a phenyl group unsubstituted or substituted with deuterium; a biphenyl group unsubstituted or substituted with deuterium; a terphenyl group unsubstituted or substituted with deuterium; a triphenylene group unsubstituted or substituted with deuterium; or a dibenzofuran group unsubstituted or substituted with deuterium.

In an exemplary embodiment of the present specification, R23 and R24 may be each independently deuterium; deuterium; a substituted or unsubstituted C1 to C60 alkyl group; a substituted or unsubstituted C6 to C60 aryl group; or a substituted or unsubstituted C2 to C60 heteroaryl group.

In an exemplary embodiment of the present specification, R23 and R24 may be each independently hydrogen; deuterium; a substituted or unsubstituted C1 to C10 alkyl group; a substituted or unsubstituted C6 to C20 aryl group; or a substituted or unsubstituted C2 to C20 heteroaryl group.

In an exemplary embodiment of the present specification, R23 and R24 may be each independently hydrogen; or deuterium.

In an exemplary embodiment of the present specification, the light emitting layer may further include the compound of Chemical Formula 2 or 3 as a P-type host in addition to the heterocyclic compound.

In an exemplary embodiment of the present specification, Chemical Formula 2 may be represented by any one of the following compounds.

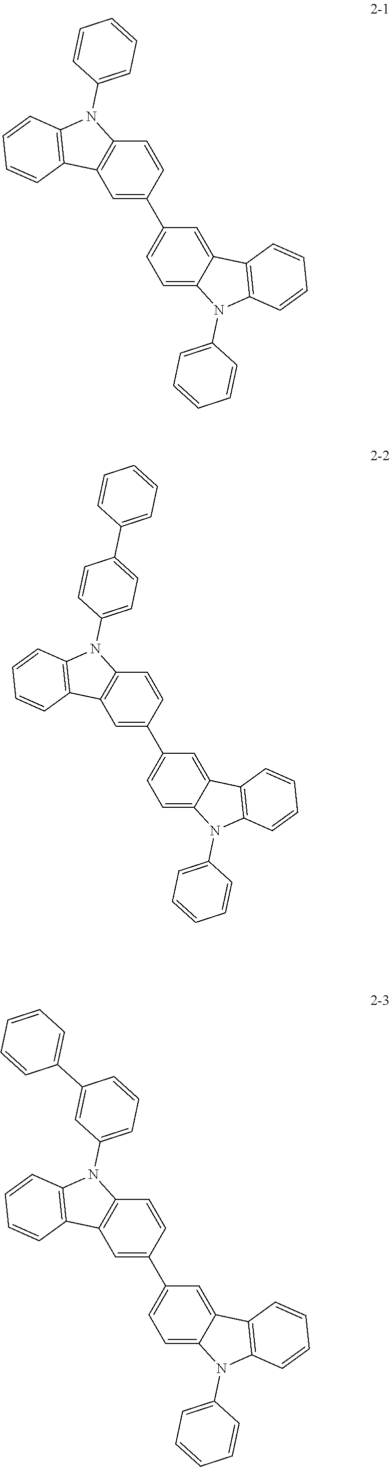 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1]

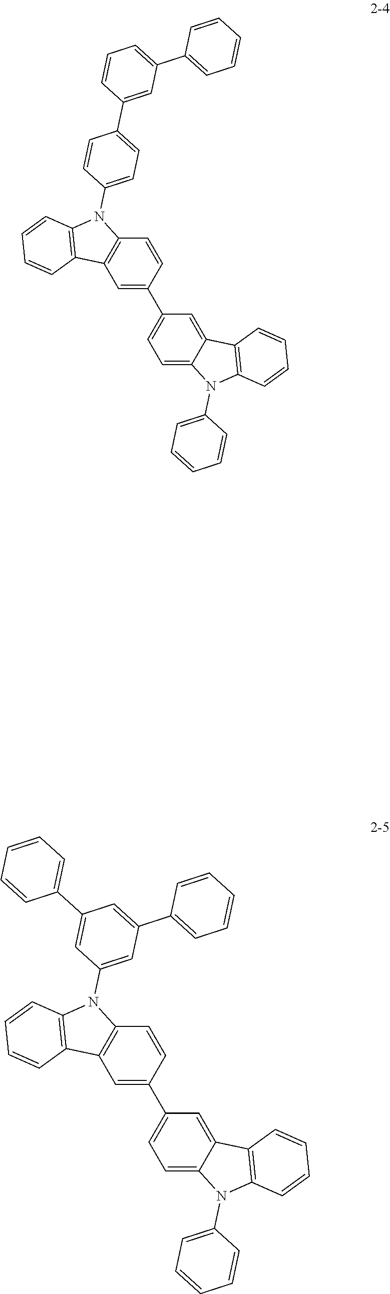 [SMILES: c1ccc(-c2cccc(-c3ccc(-n4c5ccccc5c5cc(-c6ccc7c(c6)c6ccccc6n7-c6ccccc6)ccc54)cc3)c2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

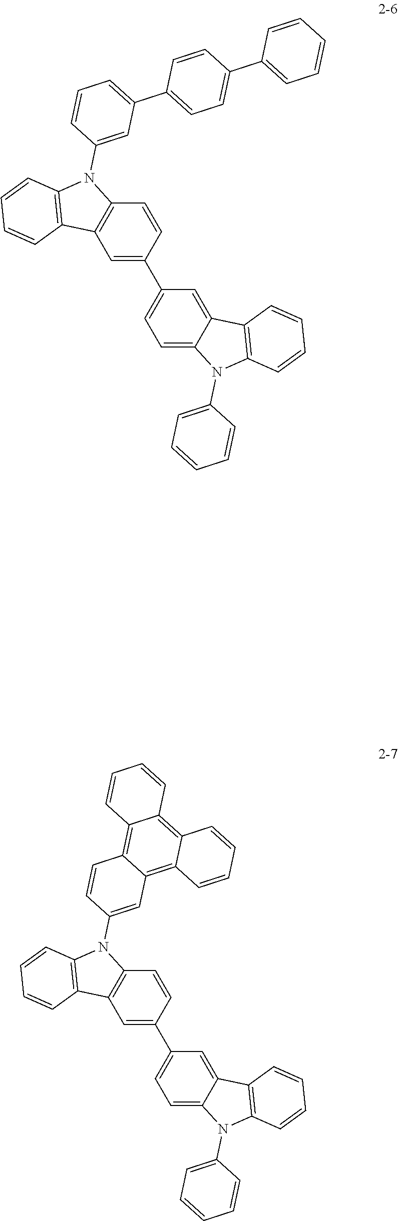 [SMILES: c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-n2c3ccccc3c3cc(-c4ccc5c(c4)c4ccccc4n5-c4ccc5c6ccccc6c6ccccc6c5c4)ccc32)cc1]

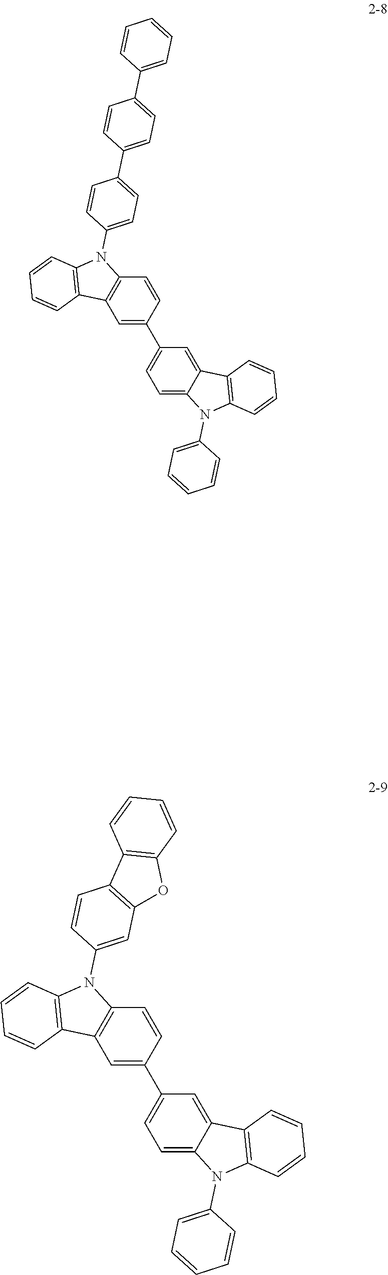 [SMILES: c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-n2c3ccccc3c3cc(-c4ccc5c(c4)c4ccccc4n5-c4ccc5c(c4)oc4ccccc45)ccc32)cc1]

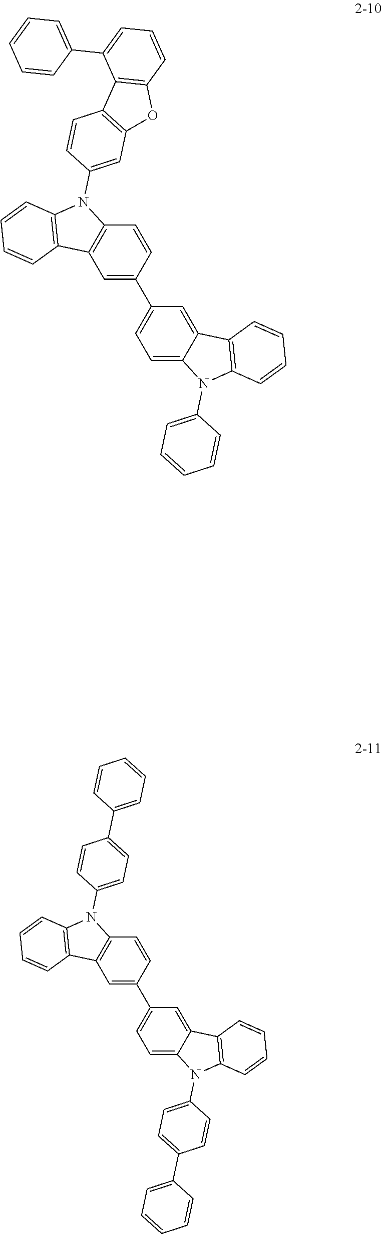 [SMILES: c1ccc(-c2cccc3oc4cc(-n5c6ccccc6c6cc(-c7ccc8c(c7)c7ccccc7n8-c7ccccc7)ccc65)ccc4c23)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

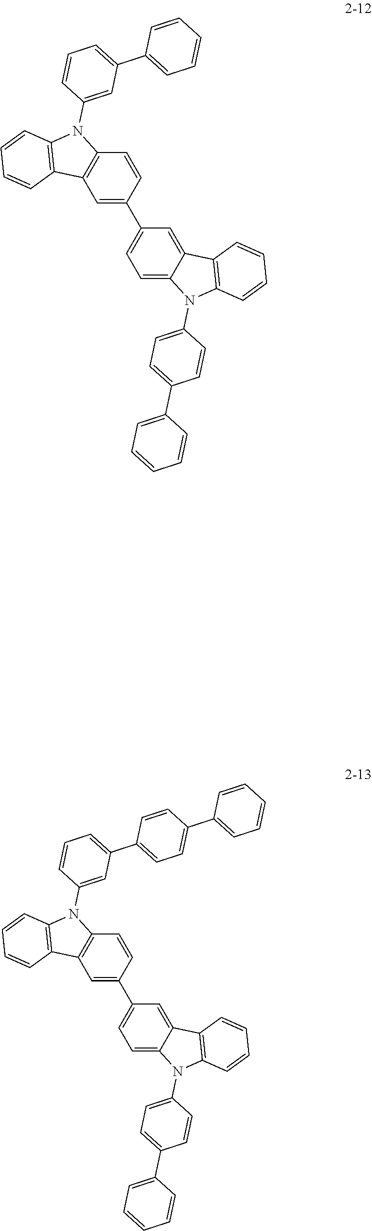 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1]

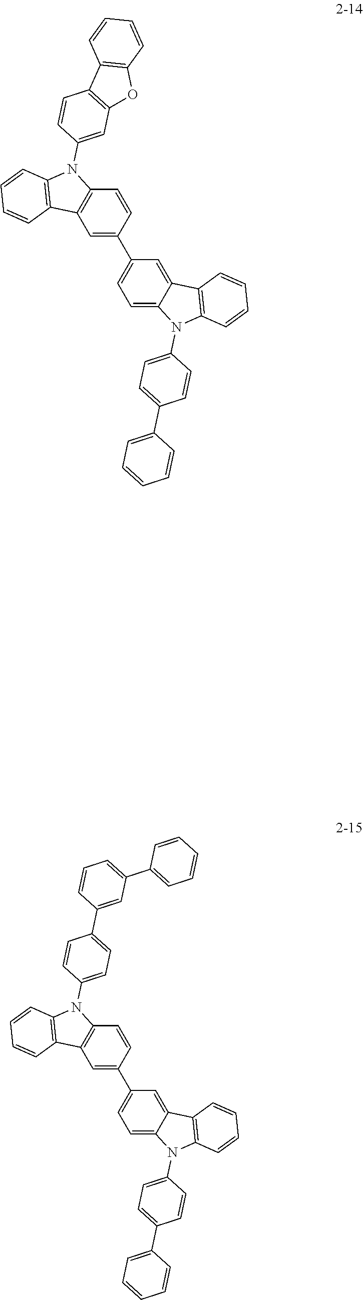 [SMILES: c1ccc(-c2ccc(-n3c4ccccc4c4cc(-c5ccc6c(c5)c5ccccc5n6-c5ccc6c(c5)oc5ccccc56)ccc43)cc2)cc1 ; c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1]

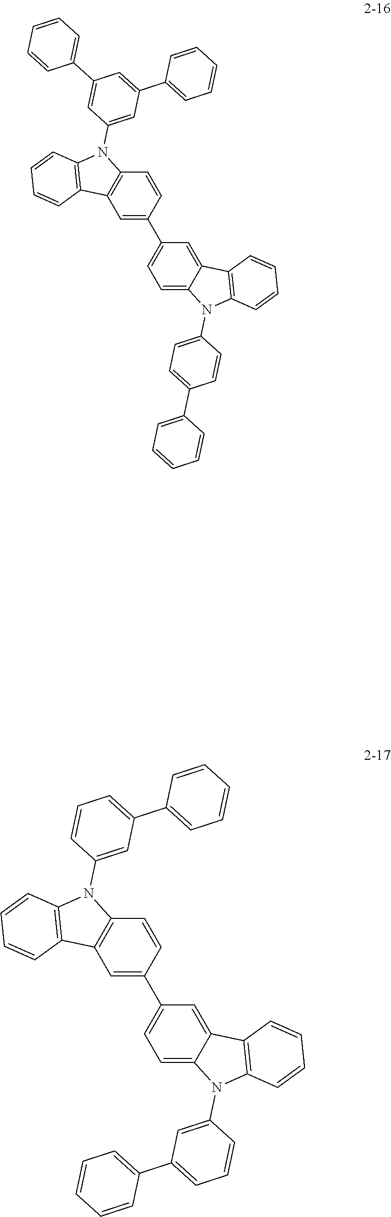 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

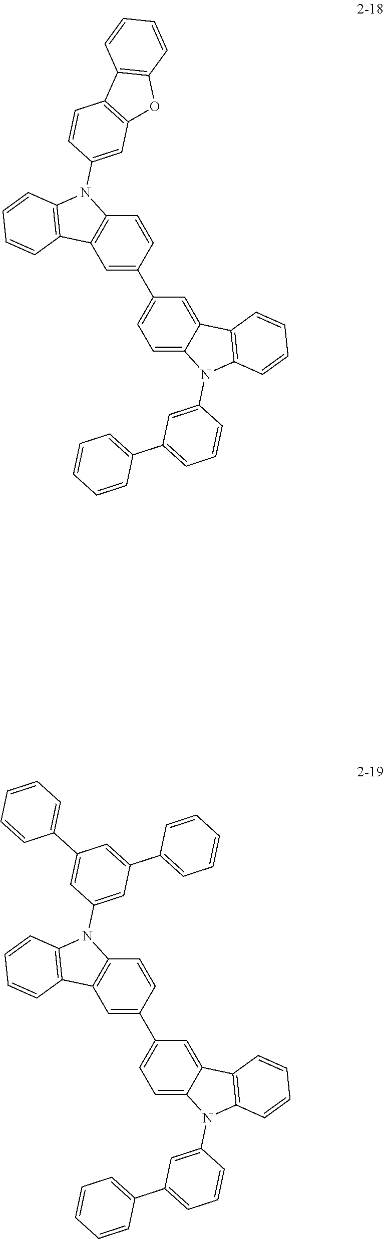 [SMILES: c1ccc(-c2cccc(-n3c4ccccc4c4cc(-c5ccc6c(c5)c5ccccc5n6-c5ccc6c(c5)oc5ccccc56)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

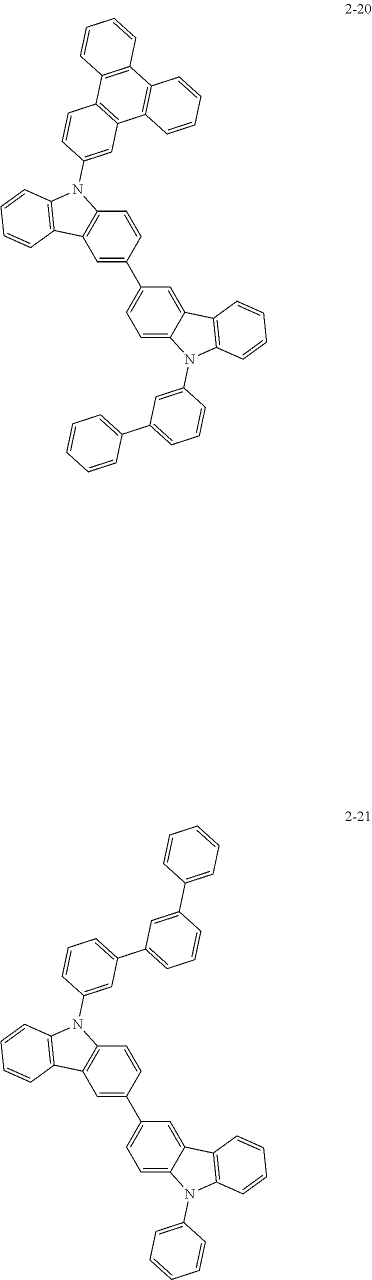 [SMILES: c1ccc(-c2cccc(-n3c4ccccc4c4cc(-c5ccc6c(c5)c5ccccc5n6-c5ccc6c7ccccc7c7ccccc7c6c5)ccc43)c2)cc1 ; c1ccc(-c2cccc(-c3cccc(-n4c5ccccc5c5cc(-c6ccc7c(c6)c6ccccc6n7-c6ccccc6)ccc54)c3)c2)cc1]

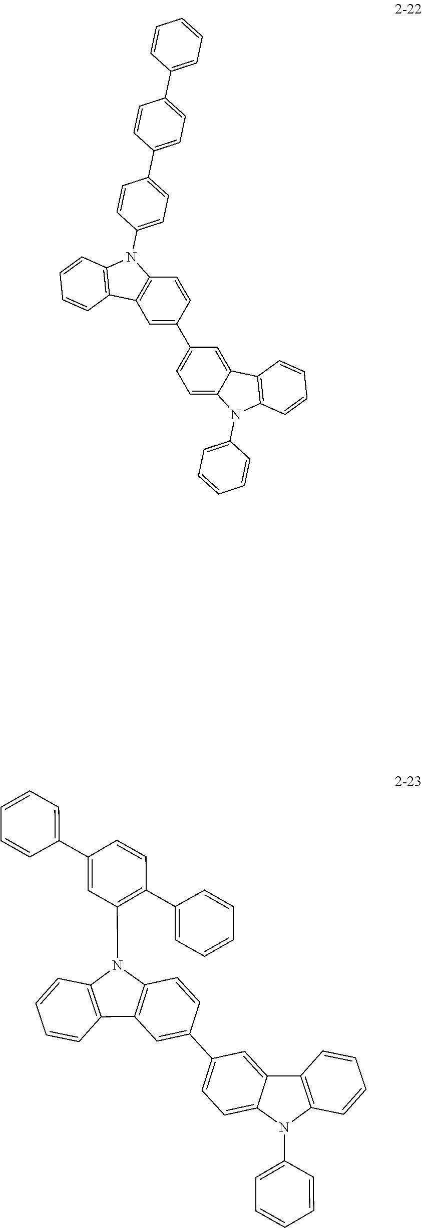 [SMILES: c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-c2ccc(-c3ccccc3)c(-n3c4ccccc4c4cc(-c5ccc6c(c5)c5ccccc5n6-c5ccccc5)ccc43)c2)cc1]

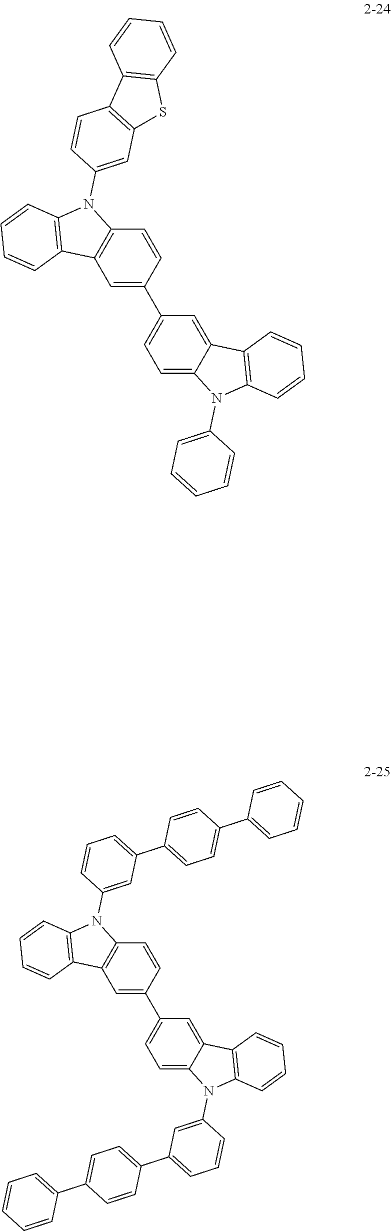 [SMILES: c1ccc(-n2c3ccccc3c3cc(-c4ccc5c(c4)c4ccccc4n5-c4ccc5c(c4)sc4ccccc45)ccc32)cc1 ; c1ccc(-c2ccc(-c3cccc(-n4c5ccccc5c5cc(-c6ccc7c(c6)c6ccccc6n7-c6cccc(-c7ccc(-c8ccccc8)cc7)c6)ccc54)c3)cc2)cc1]

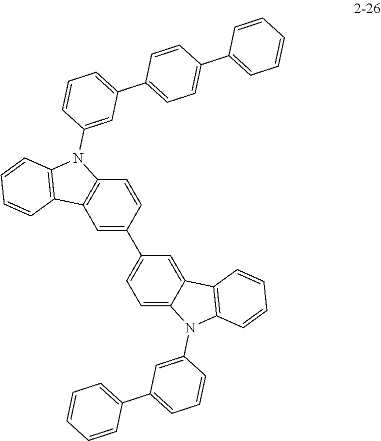 [SMILES: c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1]

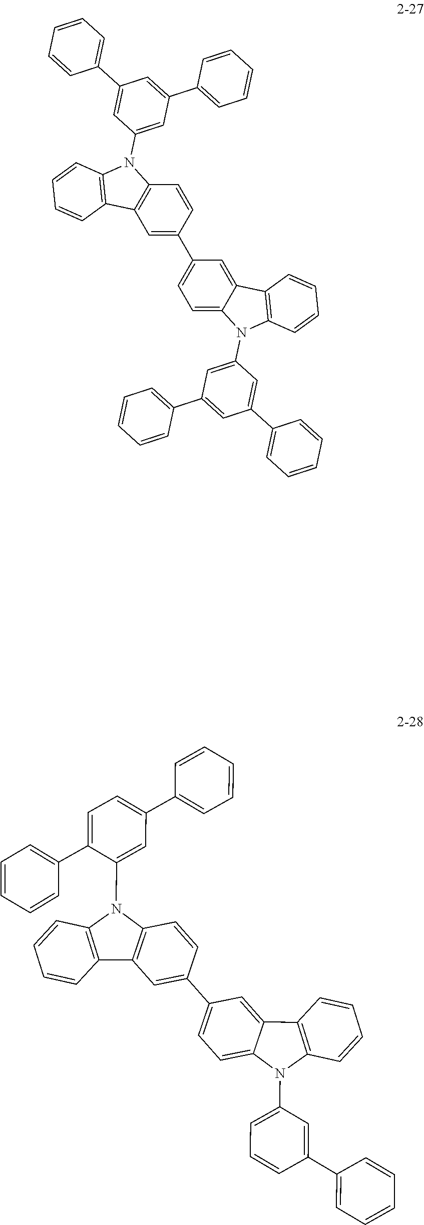 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5ccccc5n6-c5cc(-c6ccccc6)ccc5-c5ccccc5)ccc43)c2)cc1]

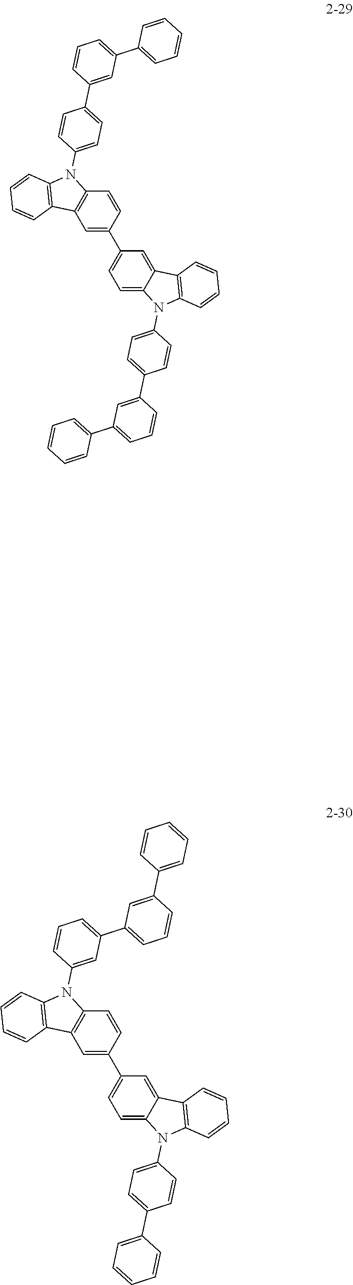 [SMILES: c1ccc(-c2cccc(-c3ccc(-n4c5ccccc5c5cc(-c6ccc7c(c6)c6ccccc6n7-c6ccc(-c7cccc(-c8ccccc8)c7)cc6)ccc54)cc3)c2)cc1 ; c1ccc(-c2ccc(-n3c4ccccc4c4cc(-c5ccc6c(c5)c5ccccc5n6-c5cccc(-c6cccc(-c7ccccc7)c6)c5)ccc43)cc2)cc1]

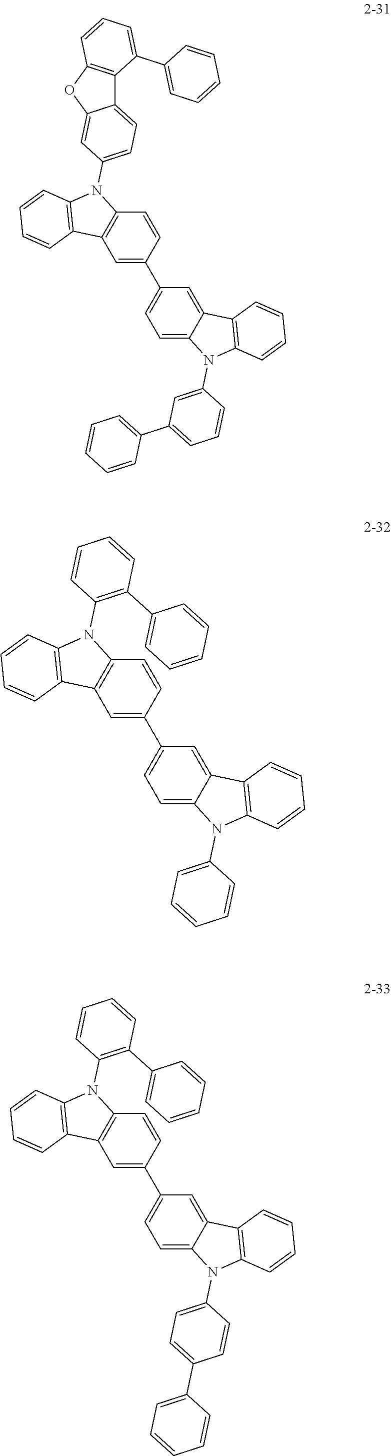 [SMILES: c1ccc(-c2cccc(-n3c4ccccc4c4cc(-c5ccc6c(c5)c5ccccc5n6-c5ccc6c(c5)oc5cccc(-c7ccccc7)c56)ccc43)c2)cc1 ; c1ccc(-c2ccccc2-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5-c5ccccc5)ccc43)cc2)cc1]

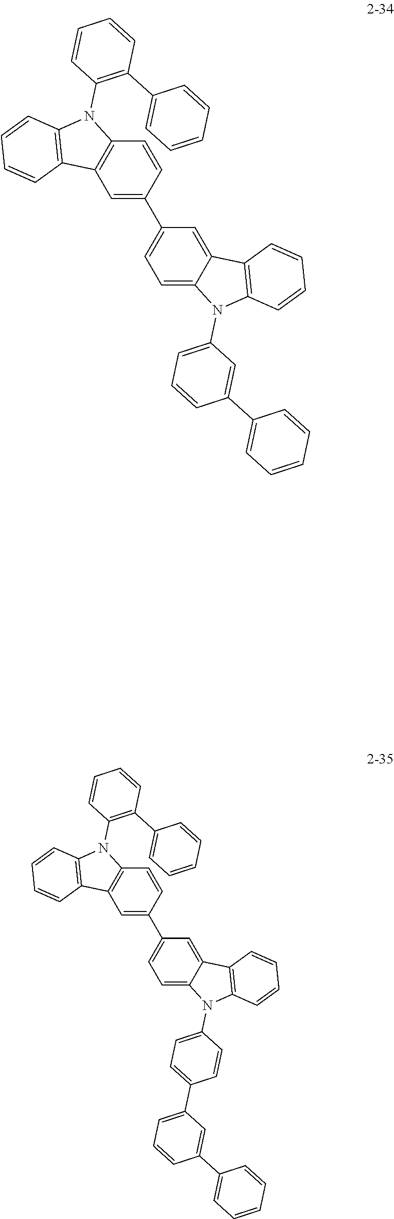 [SMILES: c1ccc(-c2cccc(-n3c4ccccc4c4cc(-c5ccc6c(c5)c5ccccc5n6-c5ccccc5-c5ccccc5)ccc43)c2)cc1 ; c1ccc(-c2cccc(-c3ccc(-n4c5ccccc5c5cc(-c6ccc7c(c6)c6ccccc6n7-c6ccccc6-c6ccccc6)ccc54)cc3)c2)cc1]

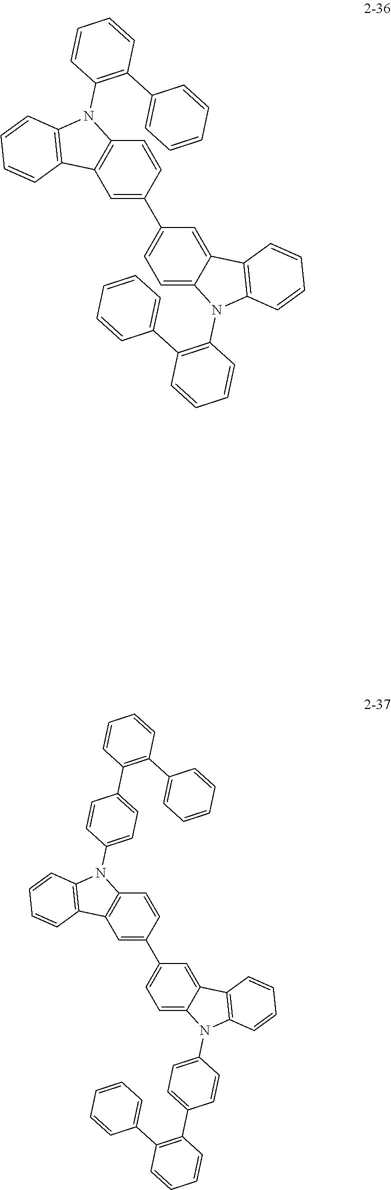 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2ccccc2-c2ccc(-n3c4ccccc4c4cc(-c5ccc6c(c5)c5ccccc5n6-c5ccc(-c6ccccc6-c6ccccc6)cc5)ccc43)cc2)cc1]

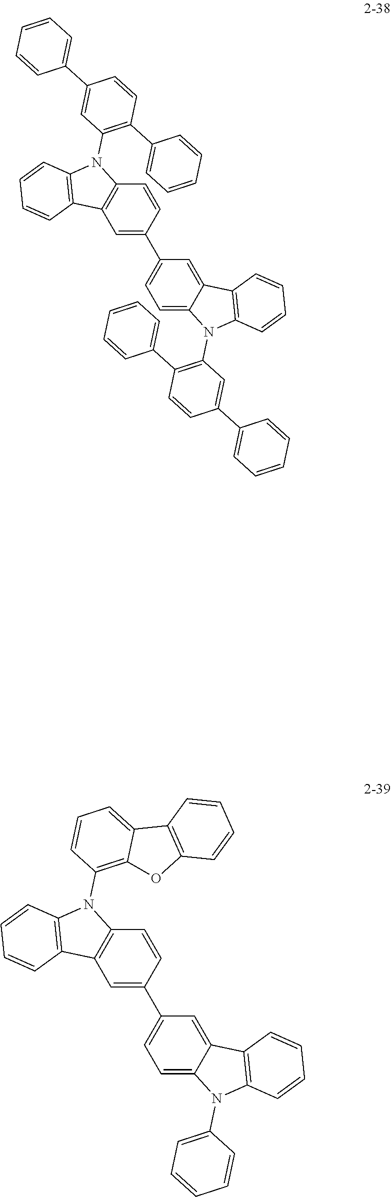 [SMILES: c1ccc(-c2ccc(-c3ccccc3)c(-n3c4ccccc4c4cc(-c5ccc6c(c5)c5ccccc5n6-c5cc(-c6ccccc6)ccc5-c5ccccc5)ccc43)c2)cc1 ; c1ccc(-n2c3ccccc3c3cc(-c4ccc5c(c4)c4ccccc4n5-c4cccc5c4oc4ccccc45)ccc32)cc1]

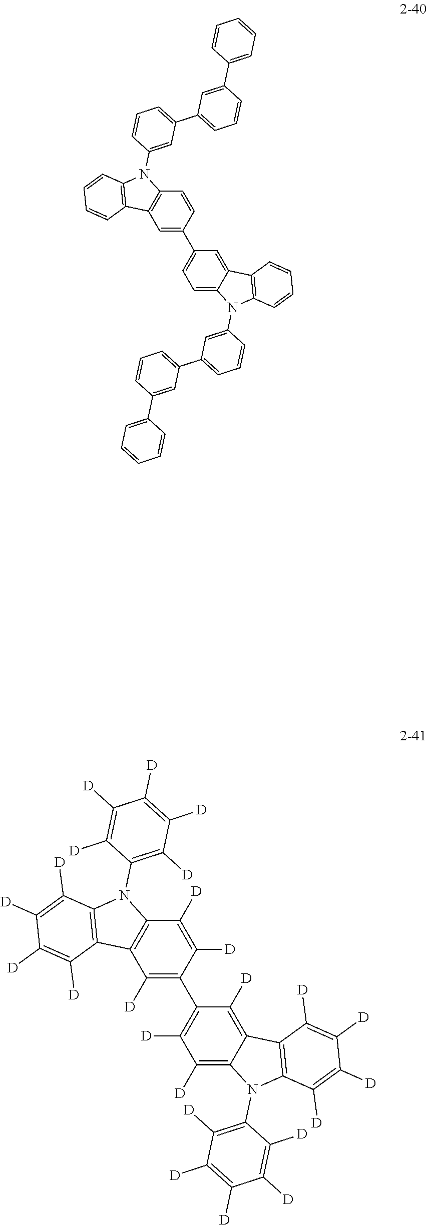 [SMILES: c1ccc(-c2cccc(-c3cccc(-n4c5ccccc5c5cc(-c6ccc7c(c6)c6ccccc6n7-c6cccc(-c7cccc(-c8ccccc8)c7)c6)ccc54)c3)c2)cc1 ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c([2H])c4[2H])c([2H])c([2H])c32)c([2H])c1[2H]]

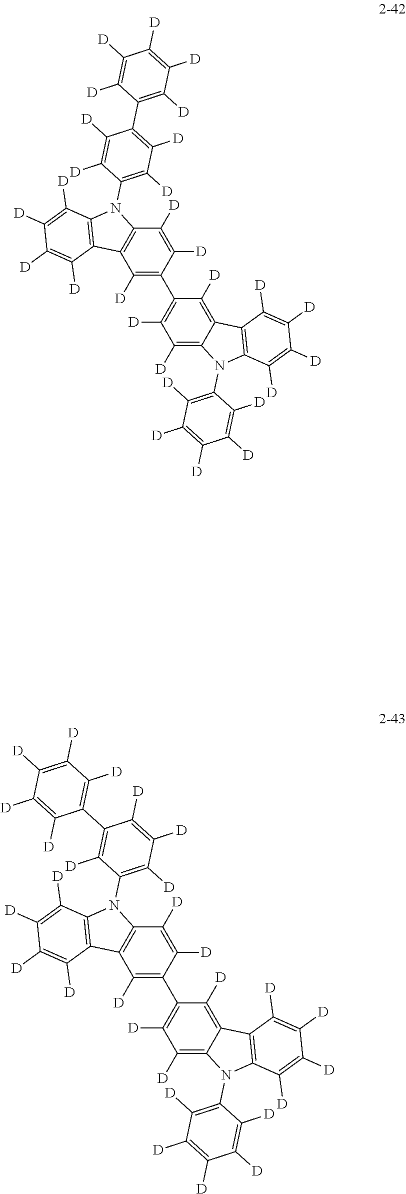 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c([2H])c2[2H])c([2H])c1[2H] ; [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c2[2H])c([2H])c1[2H]]

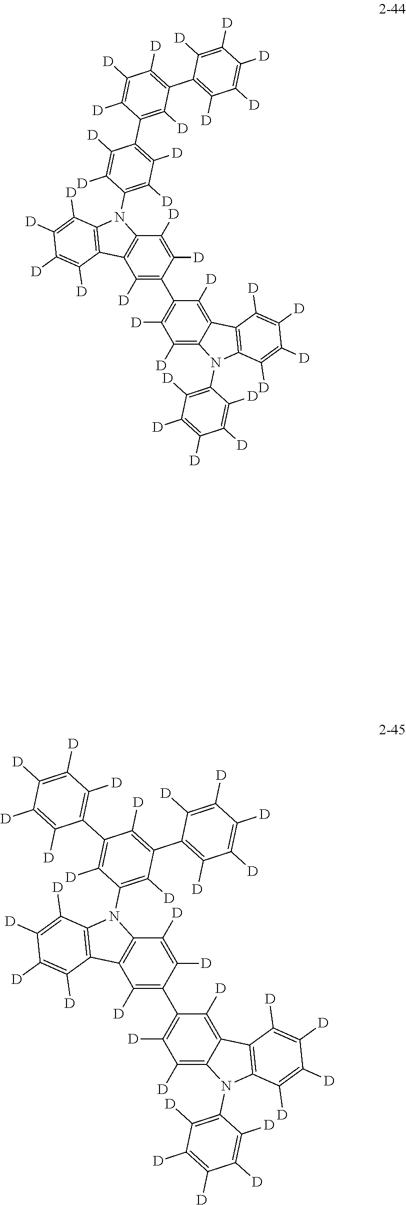

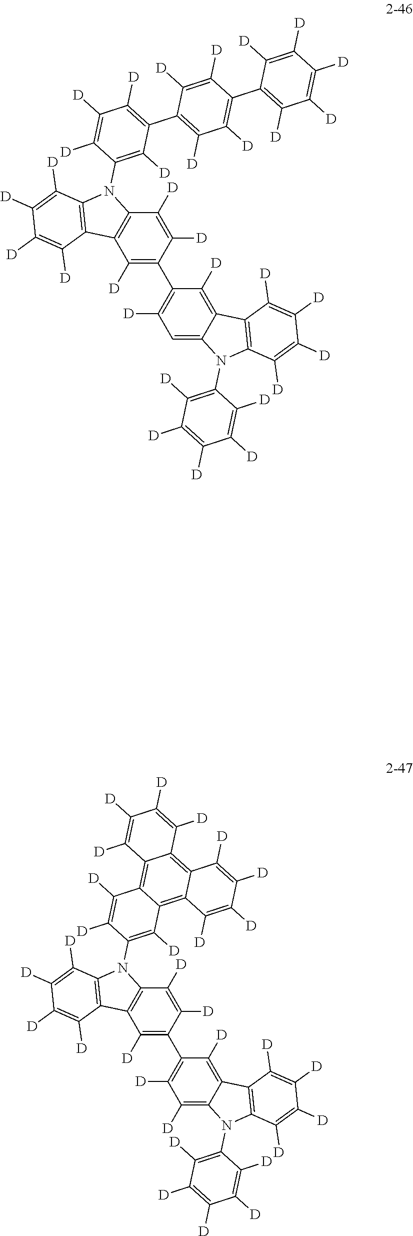

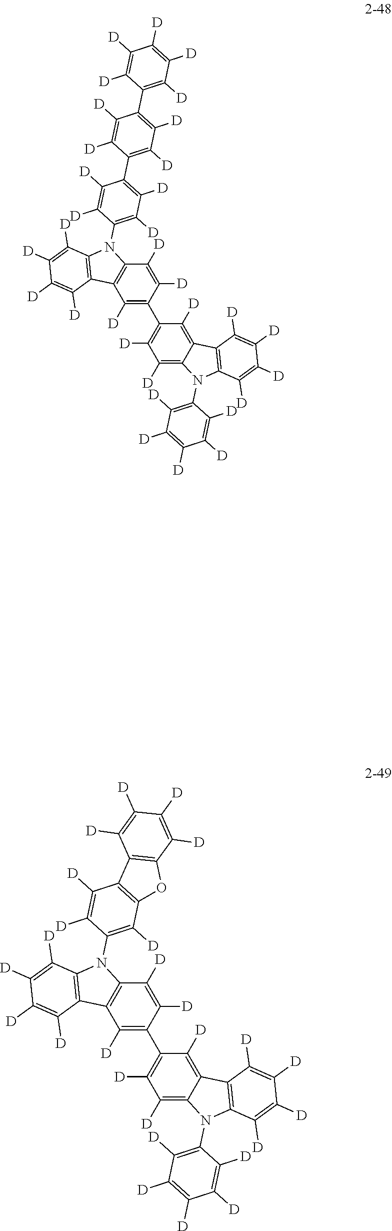 [SMILES: [2H]c1c([2H])c([2H])c(-c2c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c([2H])c6[2H])c([2H])c([2H])c54)c([2H])c3[2H])c([2H])c2[2H])c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c(oc6c([2H])c([2H])c([2H])c([2H])c65)c4[2H])c([2H])c([2H])c32)c([2H])c1[2H]]

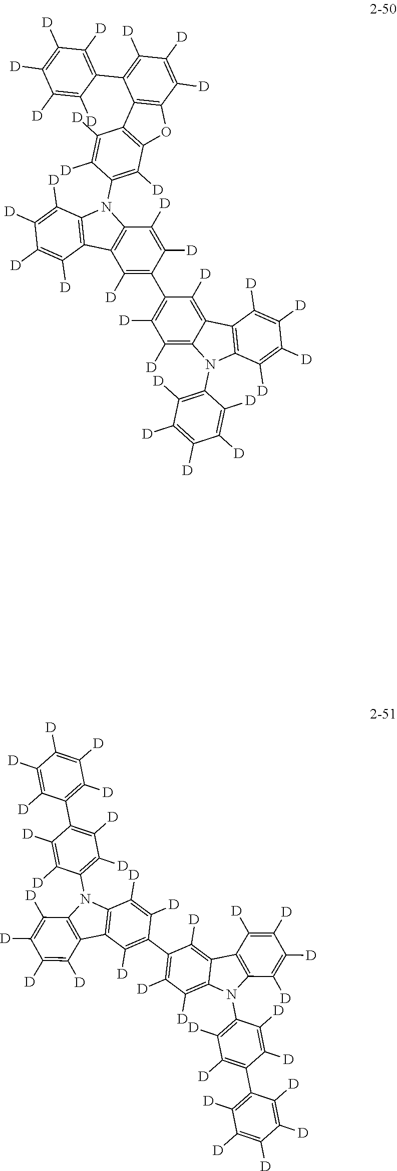

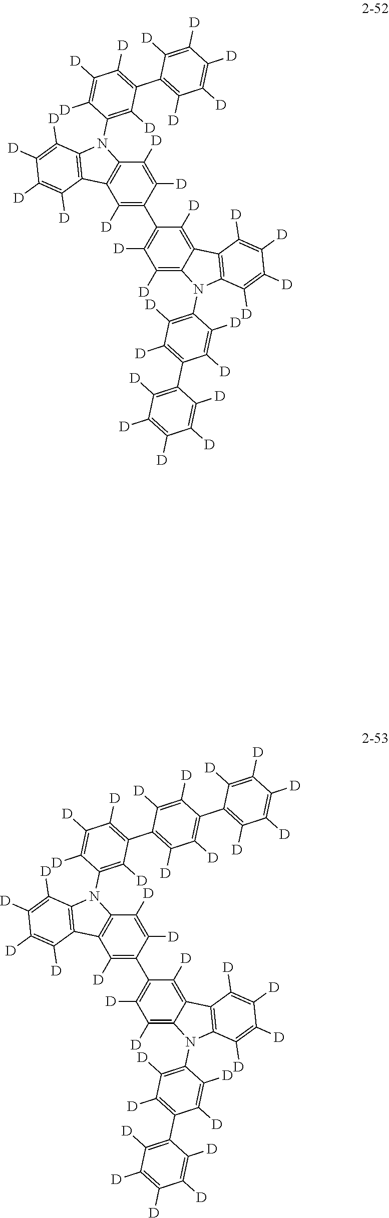

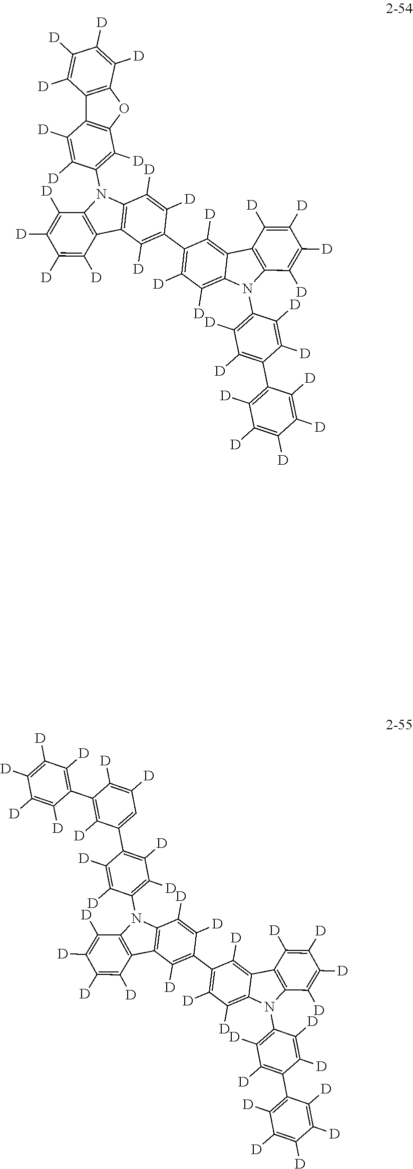

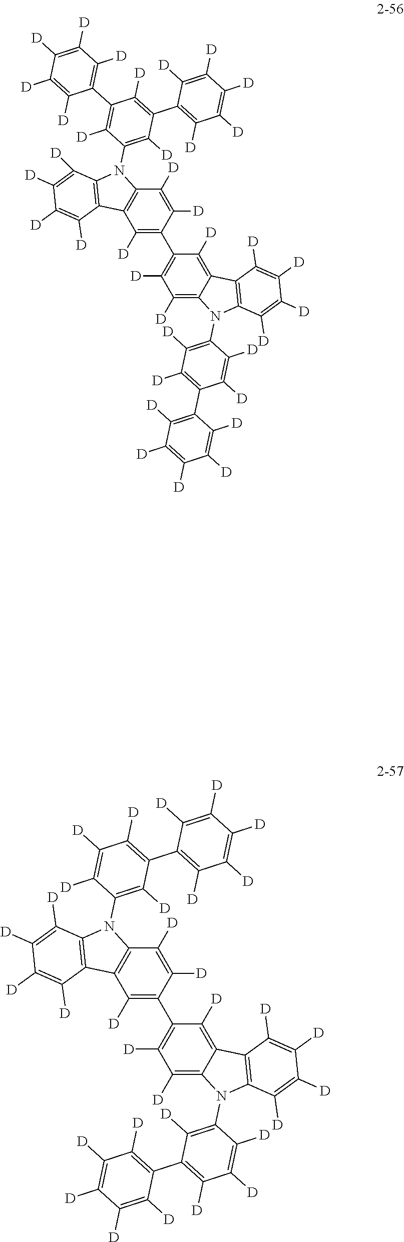

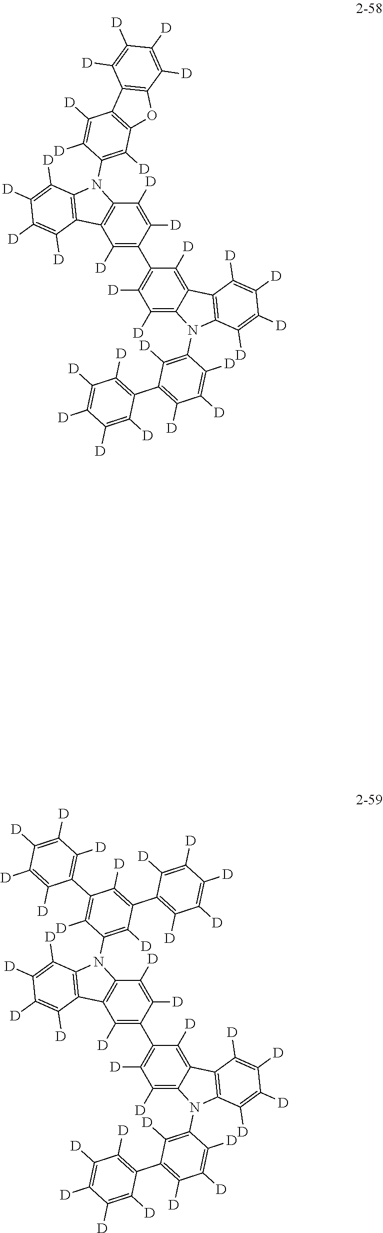

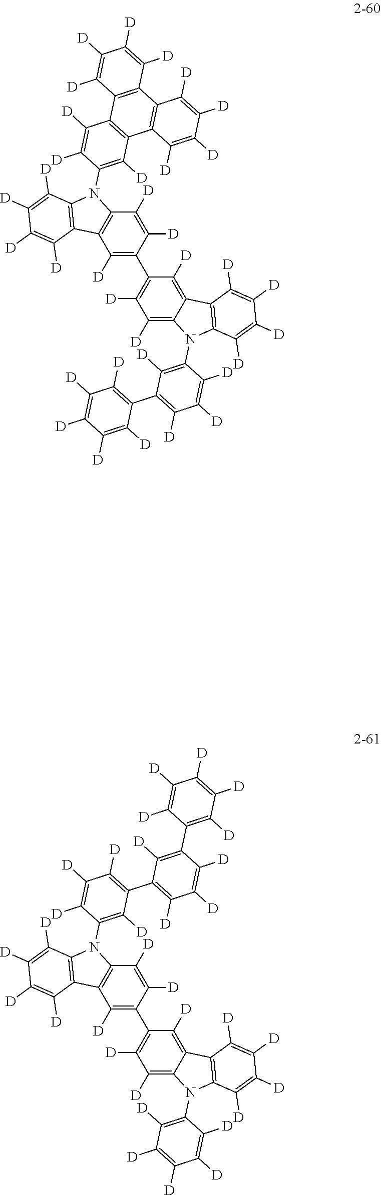

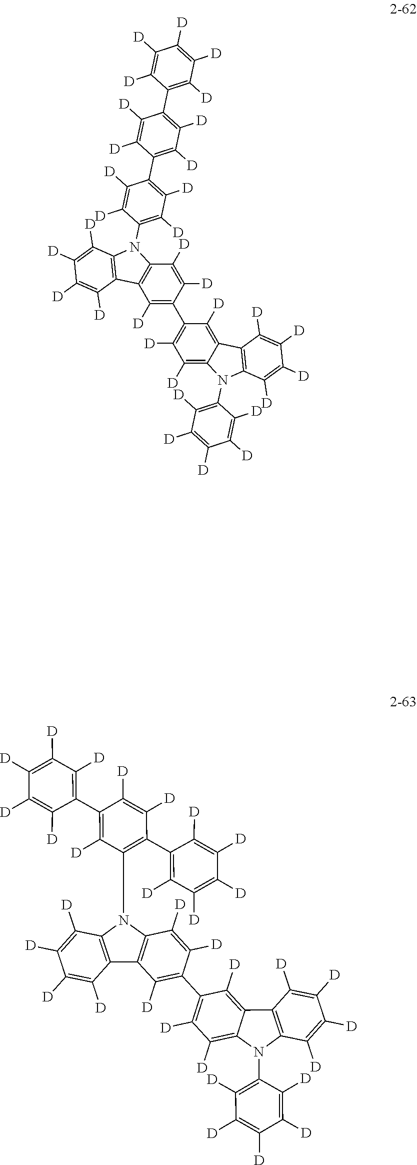

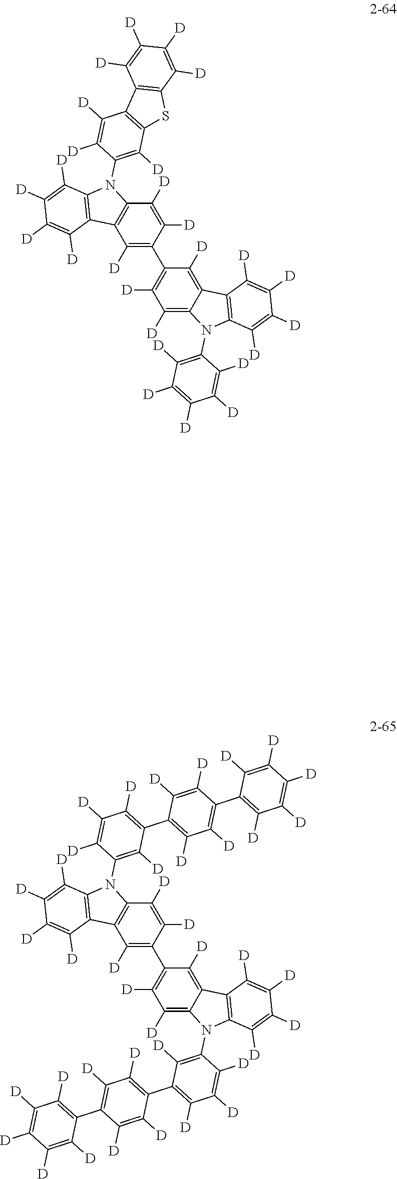

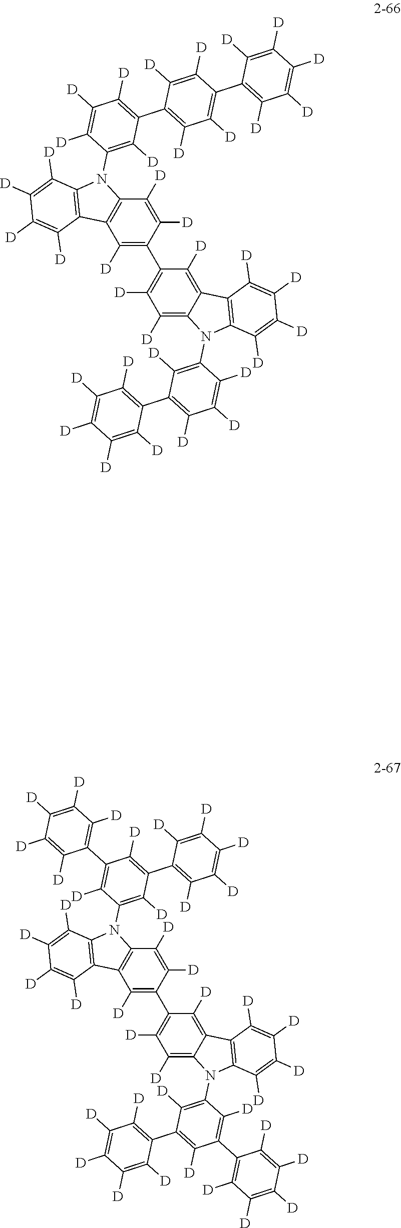

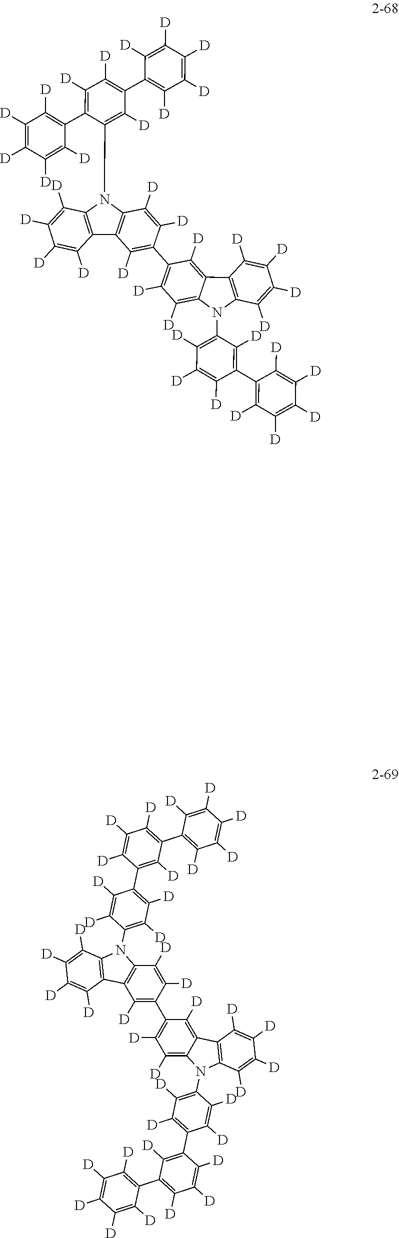

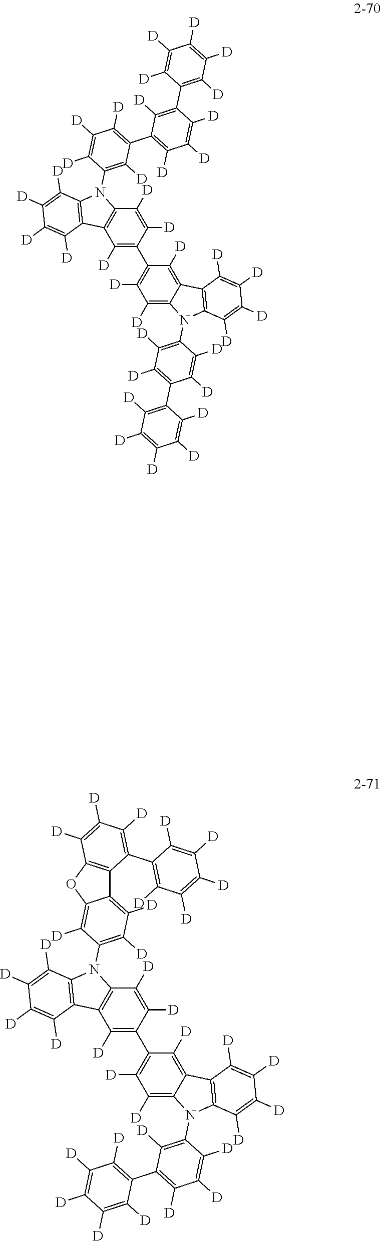

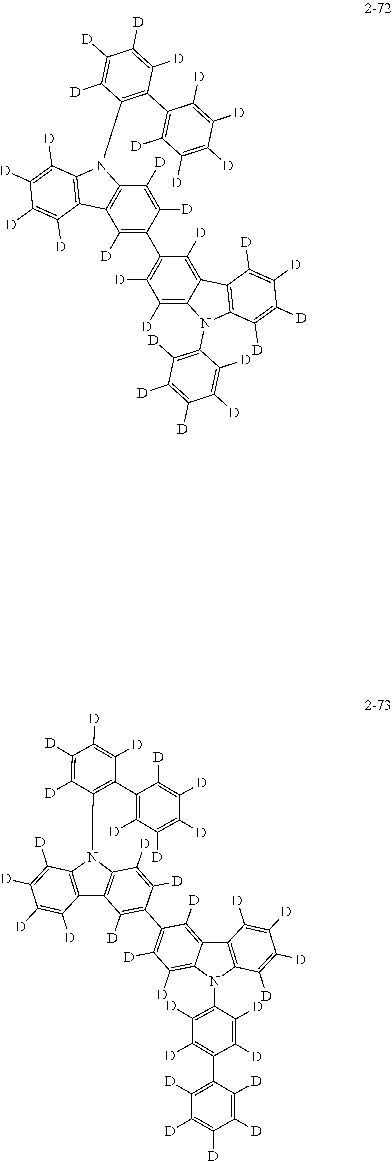 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c([2H])c4[2H])c([2H])c([2H])c32)c([2H])c1[2H] ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5-c5c([2H])c([2H])c([2H])c([2H])c5[2H])c([2H])c([2H])c43)c([2H])c2[2H])c([2H])c1[2H]]

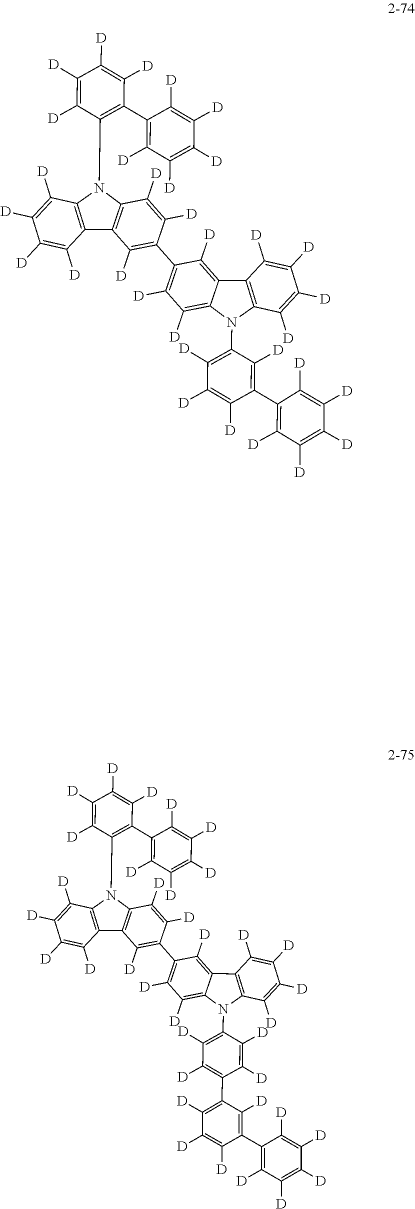

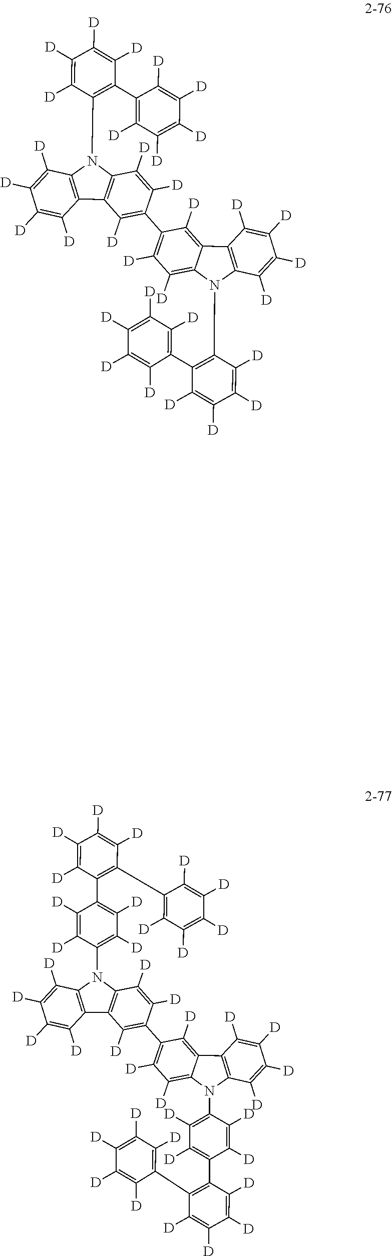

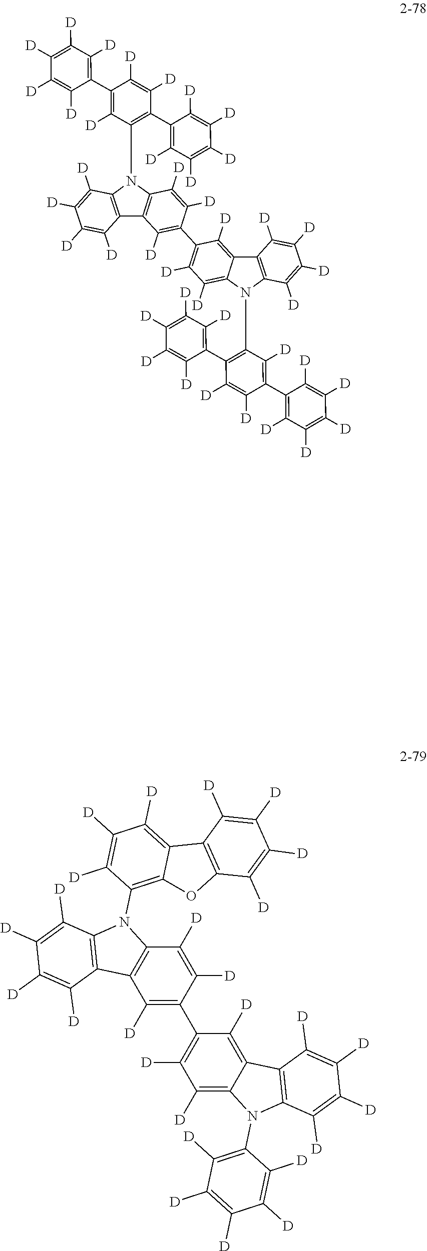

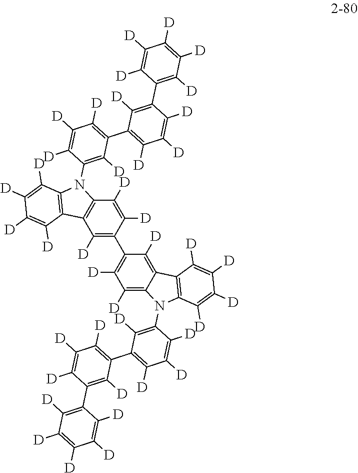 [SMILES: [2H]c1c([2H])c([2H])c(-c2c([2H])c([2H])c([2H])c(-c3c([2H])c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c(-c7c([2H])c([2H])c([2H])c(-c8c([2H])c([2H])c([2H])c([2H])c8[2H])c7[2H])c6[2H])c([2H])c([2H])c54)c3[2H])c2[2H])c([2H])c1[2H]]

In an exemplary embodiment of the present specification, Chemical Formula 3 may be represented by any one of the following compounds.

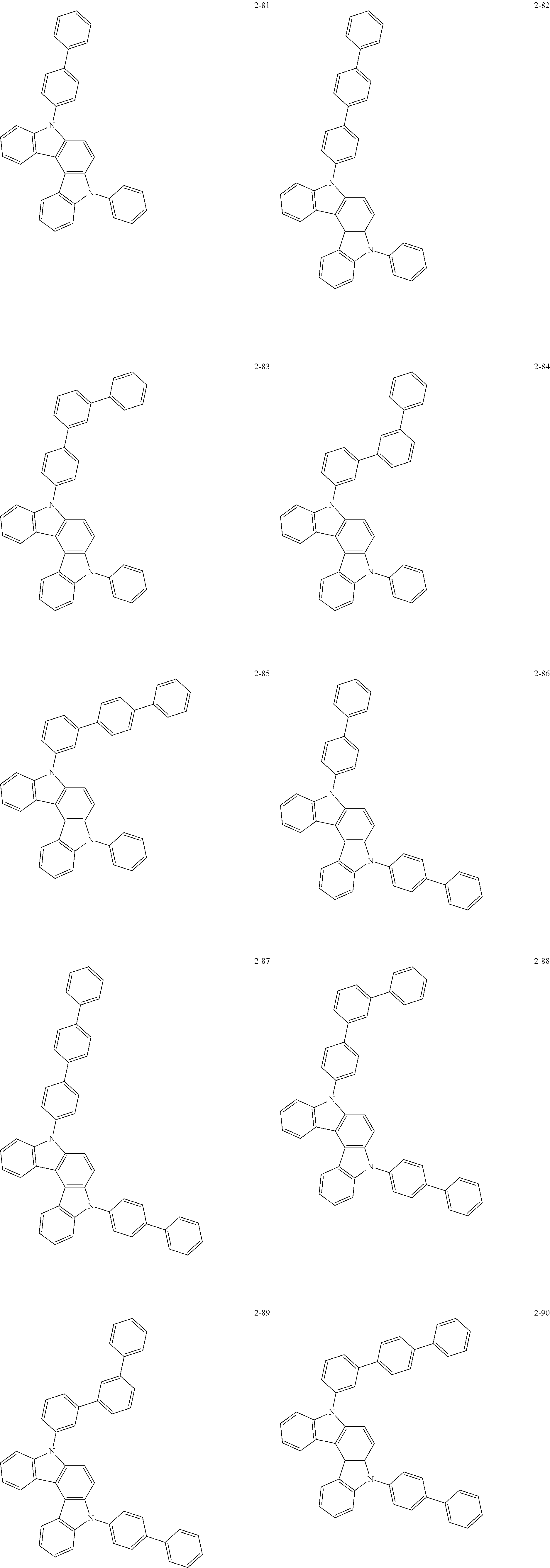

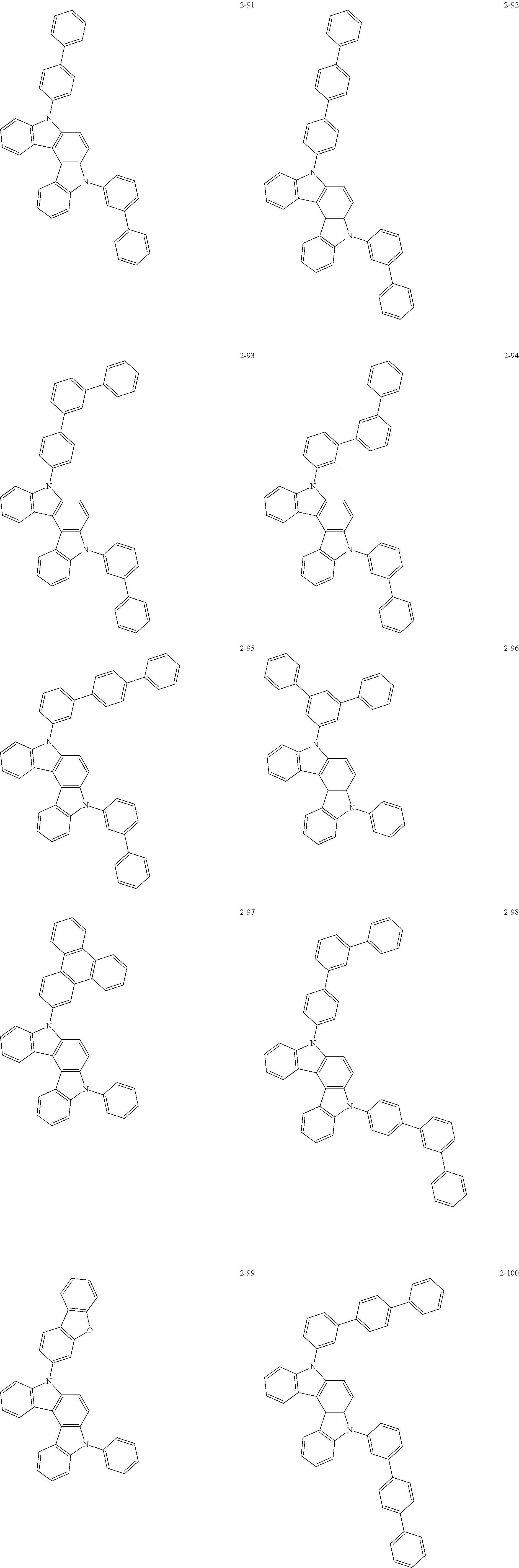

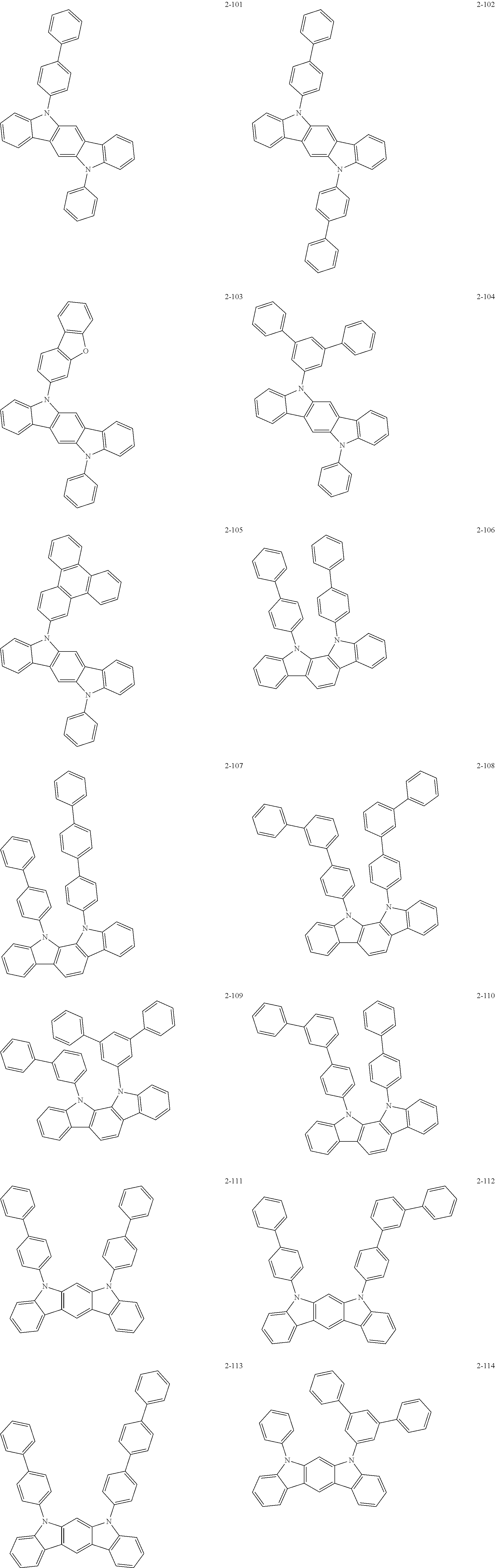

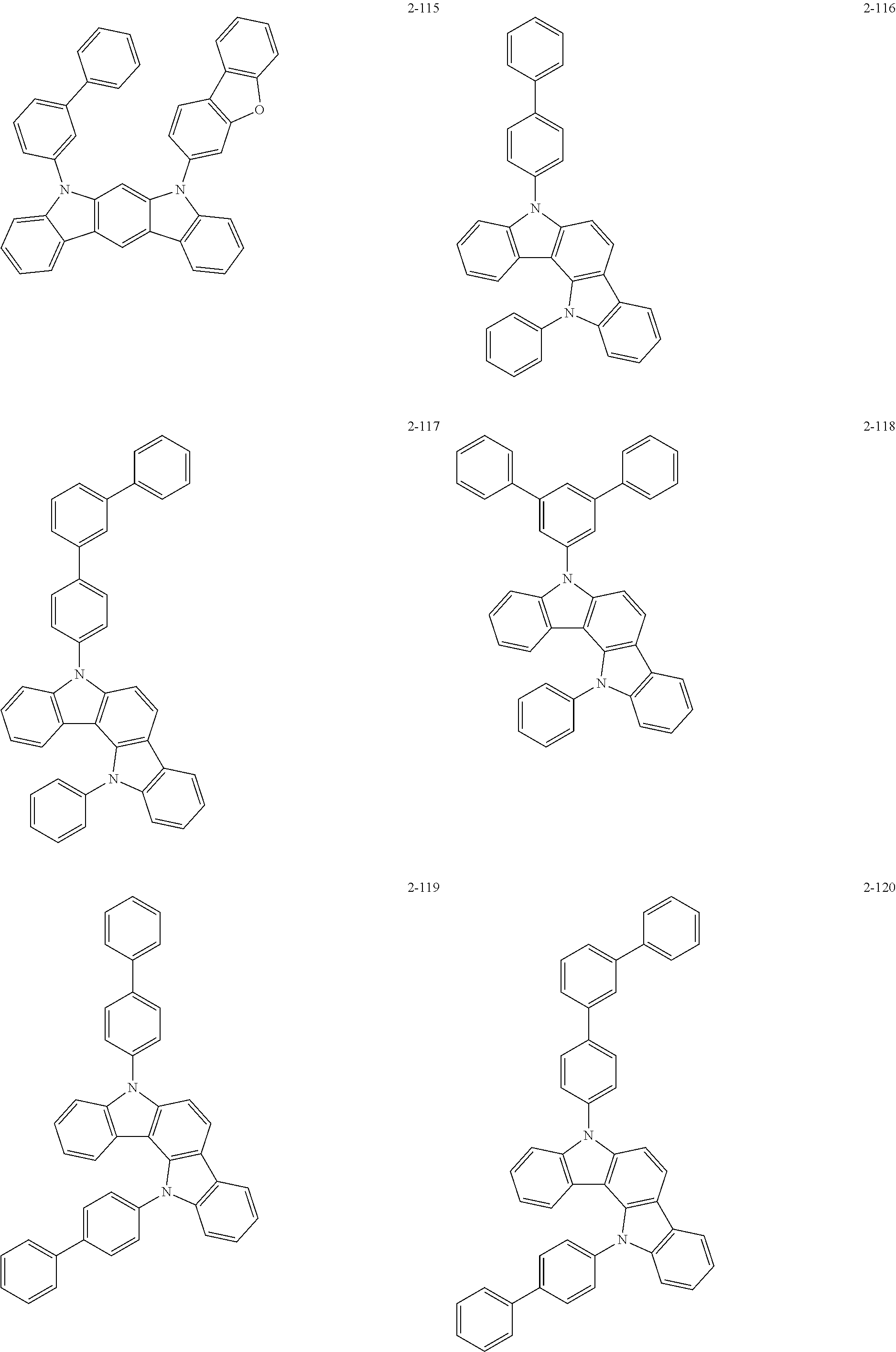 [SMILES: c1ccc(-c2cccc(-n3c4ccccc4c4cc5c6ccccc6n(-c6ccc7c(c6)oc6ccccc67)c5cc43)c2)cc1 ; c1ccc(-c2ccc(-n3c4ccccc4c4c3ccc3c5ccccc5n(-c5ccccc5)c34)cc2)cc1 ; c1ccc(-c2cccc(-c3ccc(-n4c5ccccc5c5c4ccc4c6ccccc6n(-c6ccccc6)c45)cc3)c2)cc1 ; c1ccc(-c2cc(-c3ccccc3)cc(-n3c4ccccc4c4c3ccc3c5ccccc5n(-c5ccccc5)c34)c2)cc1 ; c1ccc(-c2ccc(-n3c4ccccc4c4c3ccc3c5ccccc5n(-c5ccc(-c6ccccc6)cc5)c34)cc2)cc1 ; c1ccc(-c2ccc(-n3c4ccccc4c4ccc5c(c6ccccc6n5-c5ccc(-c6cccc(-c7ccccc7)c6)cc5)c43)cc2)cc1]

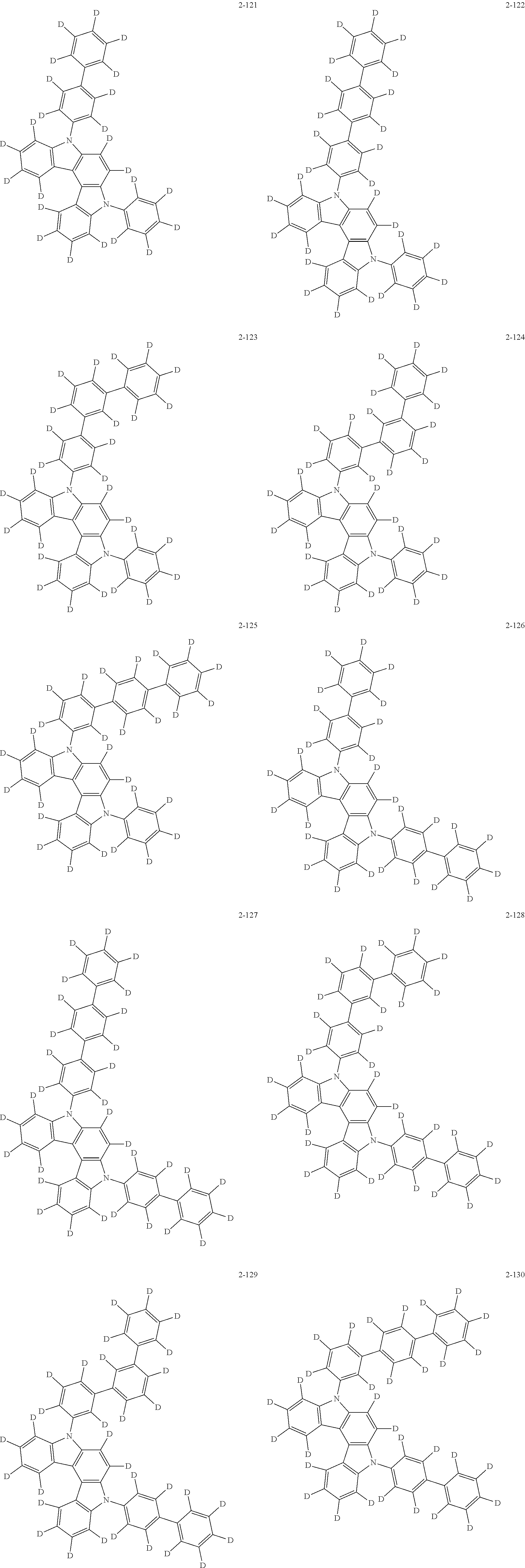

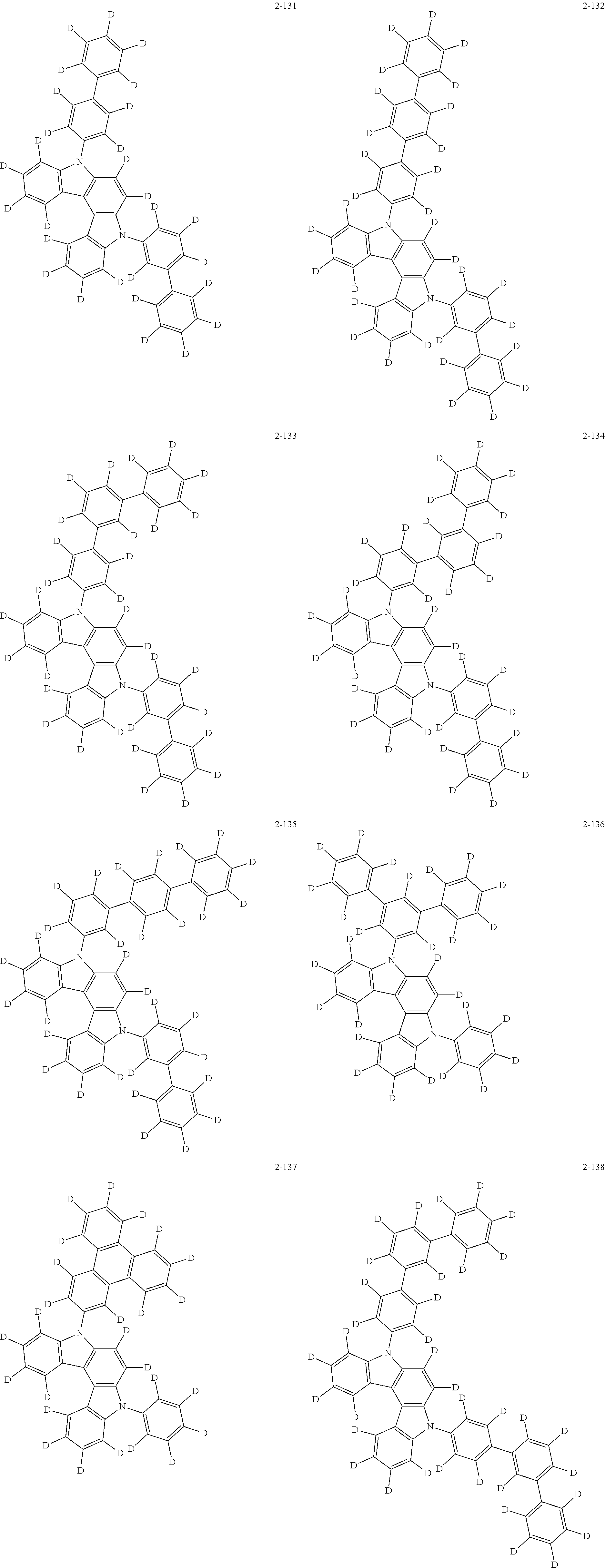

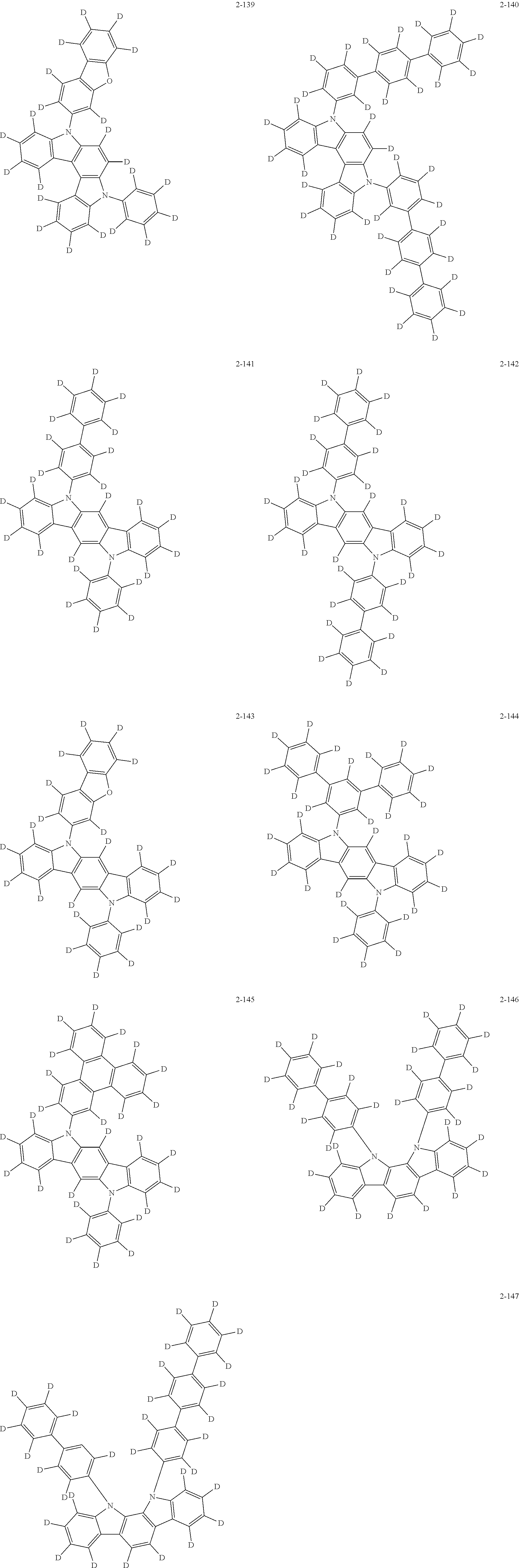

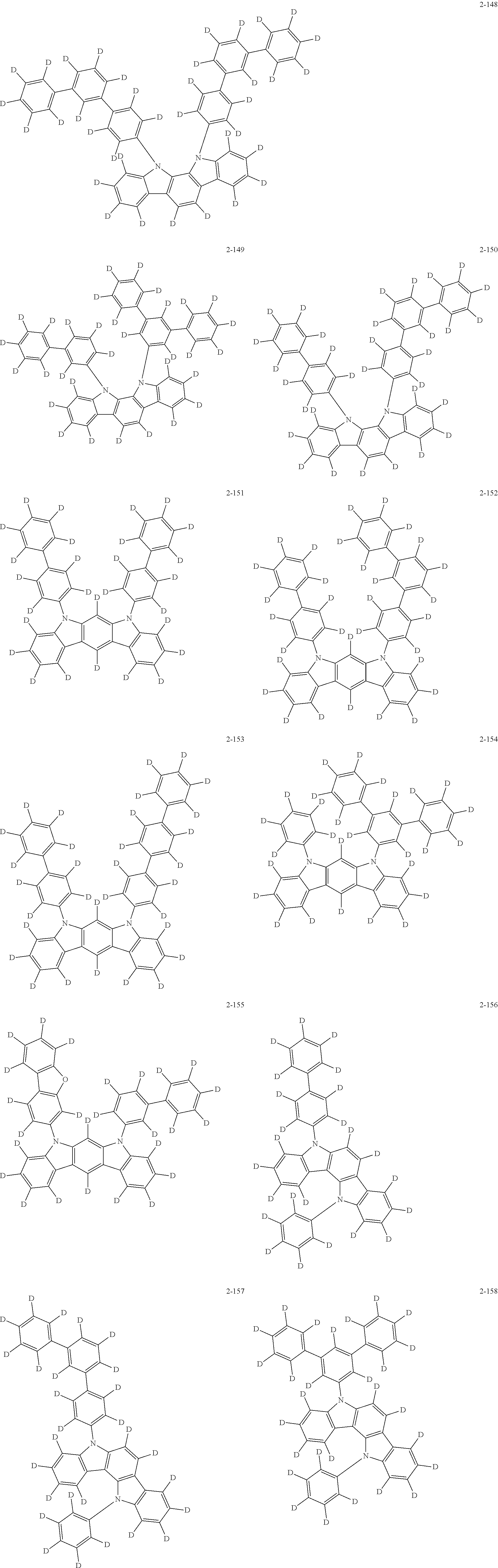

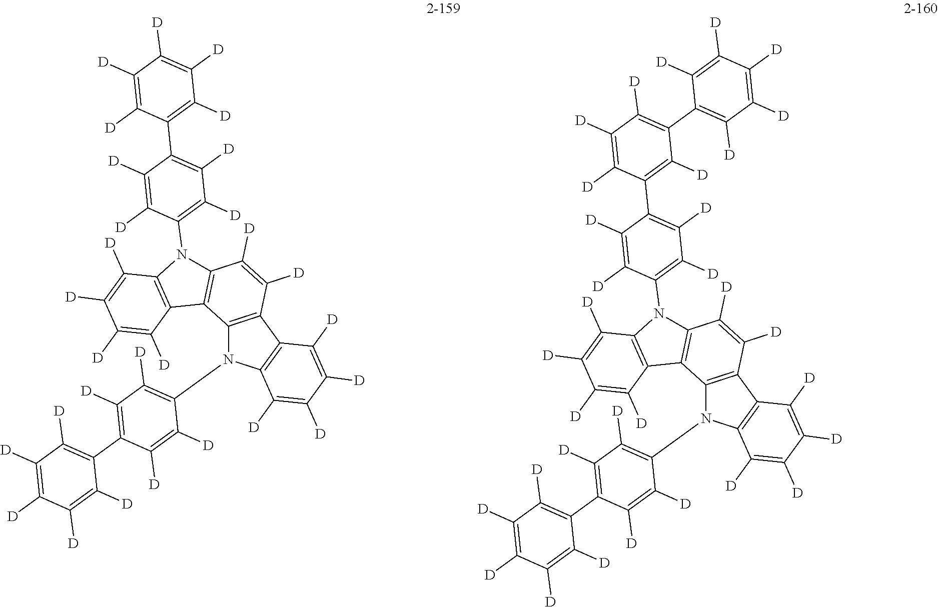 [SMILES: [2H]c1c([2H])c([2H])c(-c2c([2H])c([2H])c(-n3c4c([2H])c([2H])c([2H])c([2H])c4c4c3c([2H])c([2H])c3c5c([2H])c([2H])c([2H])c([2H])c5n(-c5c([2H])c([2H])c(-c6c([2H])c([2H])c([2H])c([2H])c6[2H])c([2H])c5[2H])c34)c([2H])c2[2H])c([2H])c1[2H] ; [2H]c1c([2H])c([2H])c(-c2c([2H])c([2H])c(-n3c4c([2H])c([2H])c([2H])c([2H])c4c4c([2H])c([2H])c5c(c6c([2H])c([2H])c([2H])c([2H])c6n5-c5c([2H])c([2H])c(-c6c([2H])c([2H])c([2H])c(-c7c([2H])c([2H])c([2H])c([2H])c7[2H])c6[2H])c([2H])c5[2H])c43)c([2H])c2[2H])c([2H])c1[2H]]

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In an exemplary embodiment of the present specification, the first electrode may be a positive electrode, and the second electrode may be a negative electrode.

In another exemplary embodiment of the present specification, the first electrode may be a negative electrode, and the second electrode may be a positive electrode.

The organic light emitting device according to an exemplary embodiment of the present specification may be manufactured by typical methods and materials for manufacturing an organic light emitting device, except that an organic material layer having one or more layers is formed by using the heterocyclic compound of the above-described Chemical Formula 1.

The heterocyclic compound of Chemical Formula 1 may be formed as an organic material layer by not only a vacuum deposition method, but also a solution application method when an organic light emitting device is manufactured. Herein, the solution coating method means spin coating, dip coating, inkjet printing, screen printing, a spray method, roll coating, and the like, but is not limited thereto.

In an exemplary embodiment of the present specification, the organic light emitting device may be a blue organic light emitting device, and the heterocyclic compound of Chemical Formula 1 may be used as a material for the blue organic light emitting device. For example, the heterocyclic compound of Chemical Formula 1 may be included in a light emitting layer of a blue organic light emitting device.

In another exemplary embodiment of the present specification, the organic light emitting device may be a green organic light emitting device, and the heterocyclic compound of Chemical Formula 1 may be used as a material for the green organic light emitting device. For example, the heterocyclic compound of Chemical Formula 1 may be included in a light emitting layer of a green organic light emitting device.

In still another exemplary embodiment of the present specification, the organic light emitting device may be a red organic light emitting device, and the heterocyclic compound of Chemical Formula 1 may be used as a material for the red organic light emitting device. For example, the heterocyclic compound of Chemical Formula 1 may be included in a light emitting layer of a red organic light emitting device.

The organic light emitting device of the present invention may further include one or more layers selected from the group consisting of a light emitting layer, a hole injection layer, a hole transport layer, an electron injection layer, an electron transport layer, an electron blocking layer, and a hole blocking layer.

FIGS. 1 to 3 exemplify the stacking sequence of the electrodes and the organic material layer of the organic light emitting device according to an exemplary embodiment of the present specification. However, the scope of the present application is not intended to be limited by these drawings, and the structure of the organic light emitting device known in the art may also be applied to the present application.

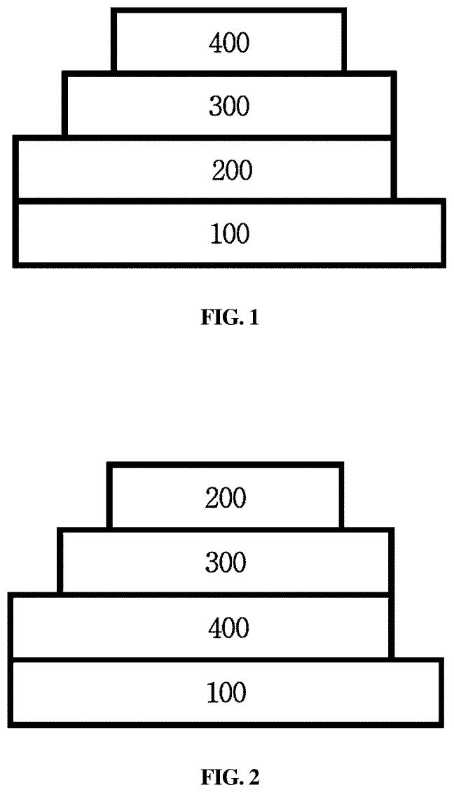

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as in FIG. 2, an organic light emitting device in which a negative electrode, an organic material layer, and a positive electrode are sequentially stacked on a substrate may also be implemented.

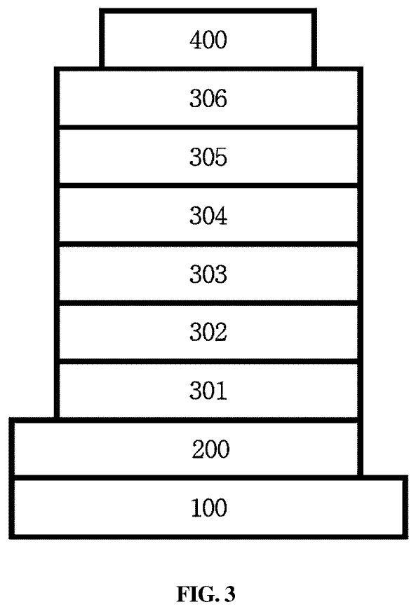

FIG. 3 exemplifies a case where an organic material layer is a multilayer. An organic light emitting device according to FIG. 3 includes a hole injection layer 301, a hole transport layer 302, an electron blocking layer 303, a light emitting layer 304, an electron transport layer 305, and an electron injection layer 306. However, the scope of the present application is not limited by the stacking structure as described above, and if necessary, the other layers except for the light emitting layer may be omitted, and another necessary functional layer may be further added.

An organic material layer including the heterocyclic compound of Chemical Formula 1 may additionally include other materials, if necessary.

In the organic light emitting device according to an exemplary embodiment of the present specification, materials other than the heterocyclic compound of Chemical Formula 1 will be exemplified below, but these materials are illustrative only and are not for limiting the scope of the present application, and may be replaced with materials publicly known in the art.

As a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene] (PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrenesulfonate), and the like.

As a hole transport material, a pyrazoline derivative, an arylamine-based derivative, a stilbene derivative, a triphenyldiamine derivative, and the like may be used, and a low-molecular weight or polymer material may also be used.

As an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials are deposited and used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, any two or more materials from N-type host materials or P-type host materials may be selected and used as a host material for a light emitting layer.

The organic light emitting device according to an exemplary embodiment of the present specification may be a top emission type, a bottom emission type, or a dual emission type according to the material to be used.

The heterocyclic compound according to an exemplary embodiment of the present specification may act even in organic electronic devices including organic solar cells, organic photoconductors, organic transistors, and the like, based on the principle similar to those applied to organic light emitting devices.

In addition, it is possible to finely adjust an energy band-gap by introducing various substituents into the structure of Chemical Formula 1, and meanwhile, it is possible to improve characteristics at the interface between organic materials and diversify the use of the material.

In an exemplary embodiment of the present specification, provided is a composition for forming an organic material layer, including the heterocyclic compound.

In an exemplary embodiment of the present specification, the composition for forming an organic material layer may further include the compound of Chemical Formula 2 or 3.

In an exemplary embodiment of the present specification, the composition for forming an organic material layer may include the heterocyclic compound and the compound of Chemical Formula 2 or 3 at a weight ratio of 1:10 to 10:1.

In an exemplary embodiment of the present specification, the composition for forming an organic material layer may include the heterocyclic compound and the compound of Chemical Formula 2 or 3 at a weight ratio of 1:5 to 5:1.

In an exemplary embodiment of the present specification, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for forming an organic material layer, including the heterocyclic compound.

In an exemplary embodiment of the present specification, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for forming an organic material layer, including the heterocyclic compound and the compound of Chemical Formula 2 or 3.

In an exemplary embodiment of the present specification, provided is a method for manufacturing an organic light emitting device, in which the forming of the organic material layer forms the organic material layer by pre-mixing the heterocyclic compound represented by Chemical Formula 1 and the compound of Chemical Formula 2 or 3, and using a thermal vacuum deposition method.

The pre-mixing means that before the heterocyclic compound represented by Chemical Formula 1 and the compound of Chemical Formula 2 or 3 are deposited onto an organic material layer, the materials are first mixed and the mixture is contained in one common container and mixed.

The pre-mixed material may be referred to as a composition for forming an organic material layer according to an exemplary embodiment of the present specification.

Hereinafter, the present specification will be described in more detail through Examples, but these Examples are provided only for exemplifying the present application, and are not intended to limit the scope of the present application.

### Preparation Example 1

### Preparation of Compound 9C

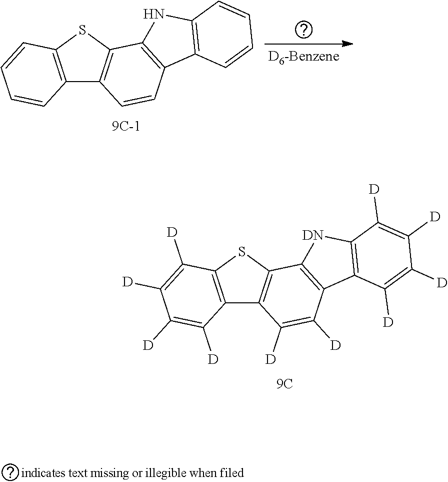

After Compound 9C-1 (12H-benzo[4,5]thieno[2,3-a]carbazole) (20 g, 73.16 mmol) was dissolved in 200 mL of D₆-benzene, triflic acid (45 mL, 512.16 mmol) was slowly added thereto. After the resulting mixture was stirred for 1 hour by increasing the reaction temperature to 60° C., the mixture was neutralized by adding a solution of Na₂CO₃ (15 g, 146.32 mmol) dissolved in 150 mL of D₂O thereto. Extraction was performed by adding an excess amount of ethyl acetate (EA) thereto, and the organic layer was dried over anhydrous MgSO₄ and then filtered through silica gel. The solvent was removed from the filtrate using a rotary evaporator to obtain 19 g of Compound 9C as a brown solid with a yield of 92% (100% D substitution rate).

A target compound was synthesized by performing the preparation in the same manner as in Preparation Example 1, except that Intermediate 1 in the following Table 1 was used instead of Compound 9C-1.

TABLE 1

| | | | | D |
|---|---|---|---|---|
| | | | | sub- |
| Com- | | | | stitu- |
| pound | | | | tion |
| No. | Intermediate 1 | Target compound | yield | rate |
| 12C | 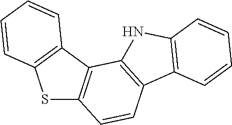 [SMILES: c1ccc2c(c1)[nH]c1c2ccc2sc3ccccc3c21] | 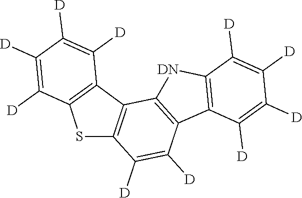 | 88% | 91% |
| 13C | 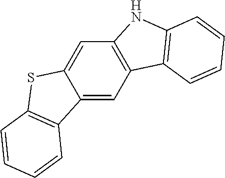 [SMILES: c1ccc2c(c1)[nH]c1cc3sc4ccccc4c3cc12] | 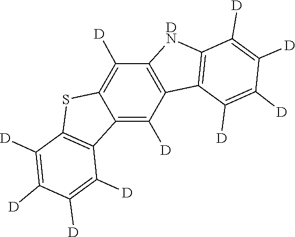 | 90% | 100% |
| 17C | 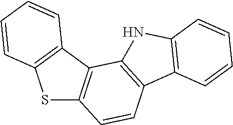 [SMILES: c1ccc2c(c1)[nH]c1c2ccc2sc3ccccc3c21] | 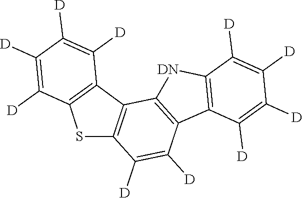 | 95% | 100% |
| 28C | 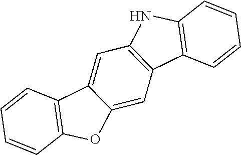 [SMILES: c1ccc2c(c1)[nH]c1cc3c(cc12)oc1ccccc13] | 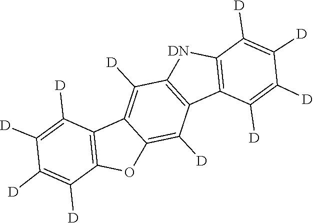 | 94% | 100% |
| 29C | 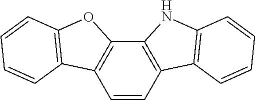 [SMILES: c1ccc2c(c1)[nH]c1c2ccc2c3ccccc3oc21] | 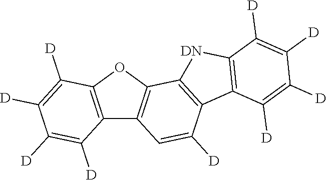 | 92% | 91% |
| 32C | 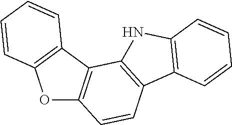 [SMILES: c1ccc2c(c1)[nH]c1c2ccc2oc3ccccc3c21] | 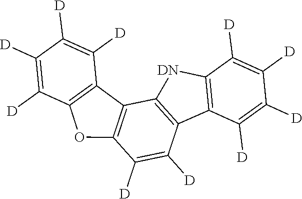 | 96% | 100% |
| 38C | 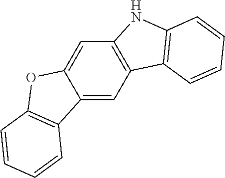 [SMILES: c1ccc2c(c1)[nH]c1cc3oc4ccccc4c3cc12] | 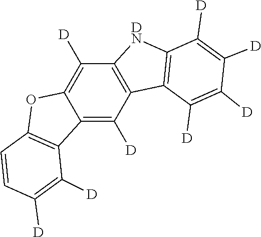 | 93% | 82% |
| 41C | 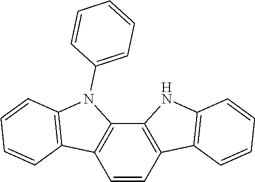 [SMILES: c1ccc(-n2c3ccccc3c3ccc4c5ccccc5[nH]c4c32)cc1] | 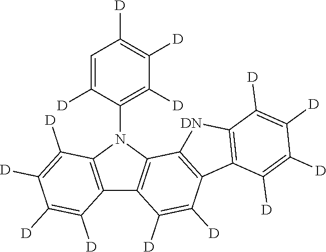 | 90% | 94% |
| 46C | 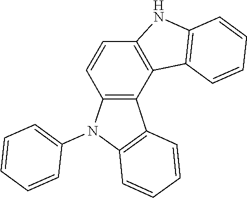 [SMILES: c1ccc(-n2c3ccccc3c3c4c(ccc32)[nH]c2ccccc24)cc1] | 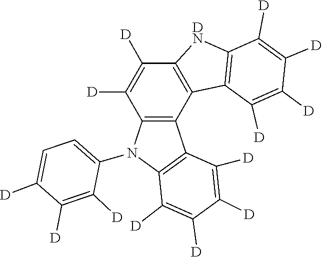 | 95% | 88% |
| 47C | 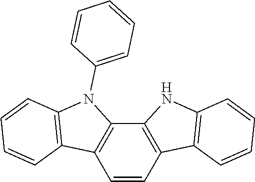 [SMILES: c1ccc(-n2c3ccccc3c3ccc4c5ccccc5[nH]c4c32)cc1] | 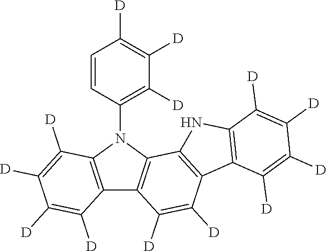 [SMILES: [2H]c1ccc(-n2c3c([2H])c([2H])c([2H])c([2H])c3c3c([2H])c([2H])c4c([nH]c5c([2H])c([2H])c([2H])c([2H])c54)c32)c([2H])c1[2H]] | 98% | 88% |
| 59C | 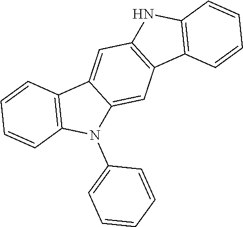 [SMILES: c1ccc(-n2c3ccccc3c3cc4[nH]c5ccccc5c4cc32)cc1] | 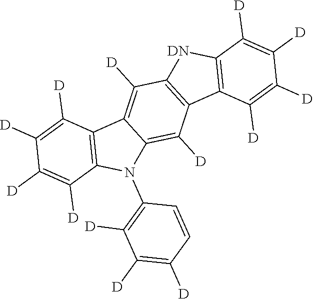 | 96% | 88% |
| 63C | 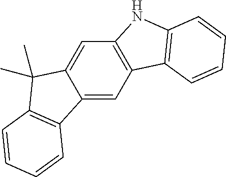 [SMILES: CC1(C)c2ccccc2-c2cc3c(cc21)[nH]c1ccccc13] | 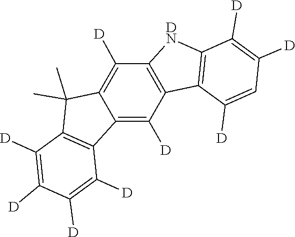 | 91% | 91% |
| 80C | 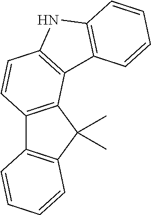 [SMILES: CC1(C)c2ccccc2-c2ccc3[nH]c4ccccc4c3c21] | 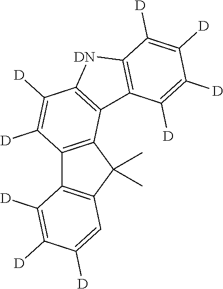 | 89% | 91% |
| 97C | 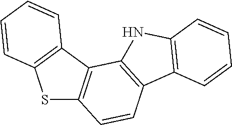 [SMILES: c1ccc2c(c1)[nH]c1c2ccc2sc3ccccc3c21] | 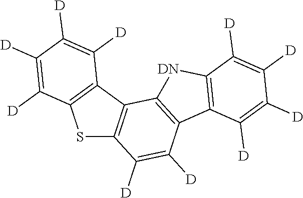 | 92% | 100% |
| 106C | 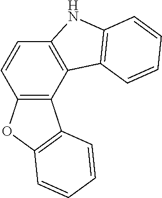 [SMILES: c1ccc2c(c1)[nH]c1ccc3oc4ccccc4c3c12] | 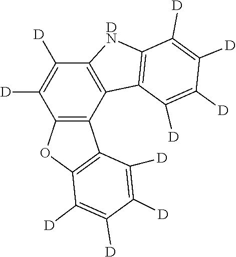 | 95% | 100% |
| 111C | 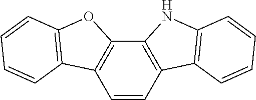 [SMILES: c1ccc2c(c1)[nH]c1c2ccc2c3ccccc3oc21] | 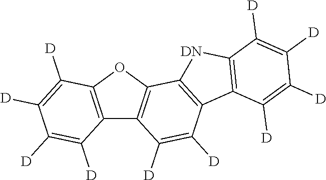 | 93% | 100% |
| 113C | 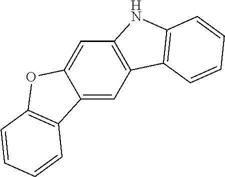 [SMILES: c1ccc2c(c1)[nH]c1cc3oc4ccccc4c3cc12] | 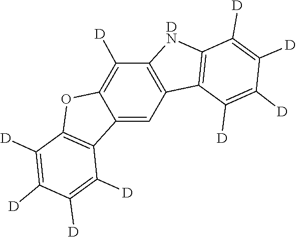 | 88% | 91% |
| 135C | 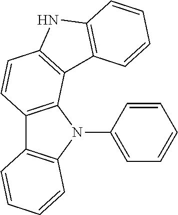 [SMILES: c1ccc(-n2c3ccccc3c3ccc4[nH]c5ccccc5c4c32)cc1] | 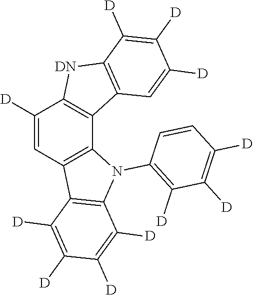 | 85% | 82% |
| 136C | 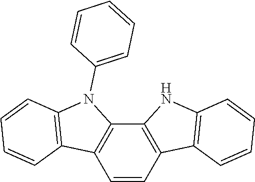 [SMILES: c1ccc(-n2c3ccccc3c3ccc4c5ccccc5[nH]c4c32)cc1] | 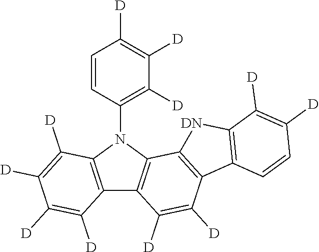 | 82% | 75% |
| 138C | 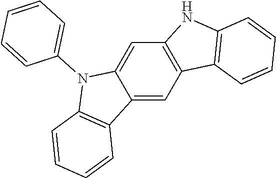 [SMILES: c1ccc(-n2c3ccccc3c3cc4c(cc32)[nH]c2ccccc24)cc1] | 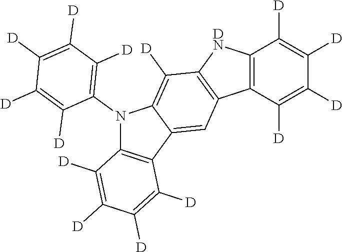 | 90% | 94% |
| 154C | 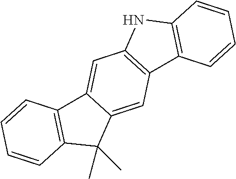 [SMILES: CC1(C)c2ccccc2-c2cc3[nH]c4ccccc4c3cc21] | 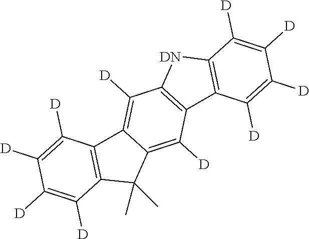 | 88% | 100% |
| 166C | 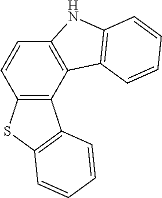 [SMILES: c1ccc2c(c1)[nH]c1ccc3sc4ccccc4c3c12] | 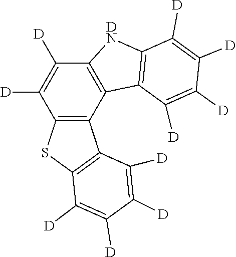 | 96% | 100% |
| 178C | 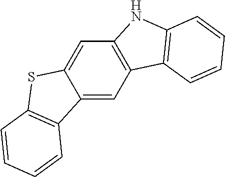 [SMILES: c1ccc2c(c1)[nH]c1cc3sc4ccccc4c3cc12] | 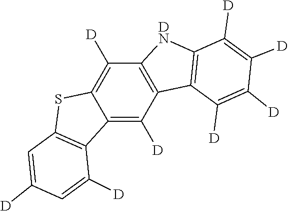 | 90% | 82% |
| 179C | 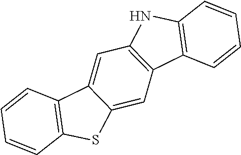 [SMILES: c1ccc2c(c1)[nH]c1cc3c(cc12)sc1ccccc13] | 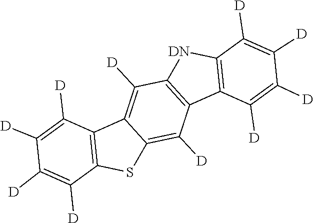 | 91% | 100% |
| 180C | 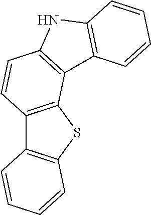 [SMILES: c1ccc2c(c1)[nH]c1ccc3c4ccccc4sc3c12] | 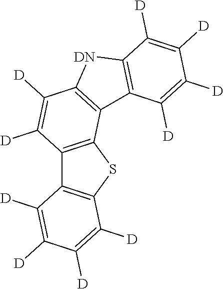 | 96% | 100% |
| 186C | 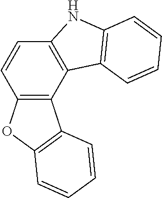 [SMILES: c1ccc2c(c1)[nH]c1ccc3oc4ccccc4c3c12] | 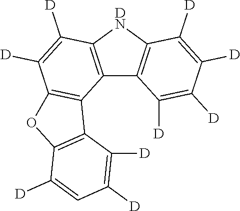 | 80% | 91% |
| 196C | 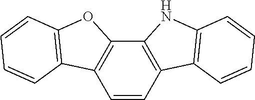 [SMILES: c1ccc2c(c1)[nH]c1c2ccc2c3ccccc3oc21] | 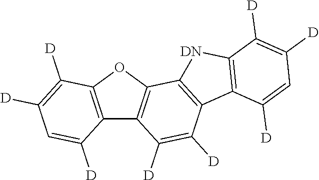 | 77% | 82% |
| 197C | 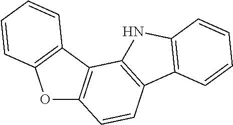 [SMILES: c1ccc2c(c1)[nH]c1c2ccc2oc3ccccc3c21] | 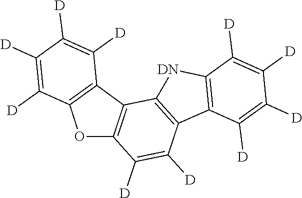 | 85% | 100% |
| 207C | 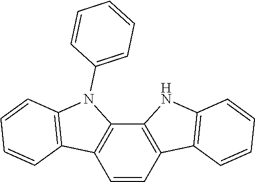 [SMILES: c1ccc(-n2c3ccccc3c3ccc4c5ccccc5[nH]c4c32)cc1] | 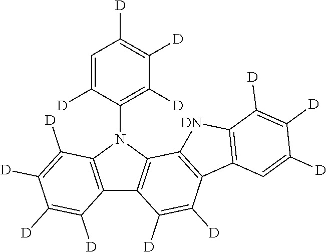 | 80% | 88% |
| 214C | 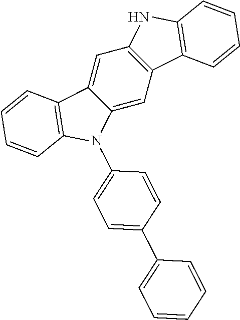 [SMILES: c1ccc(-c2ccc(-n3c4ccccc4c4cc5[nH]c6ccccc6c5cc43)cc2)cc1] | 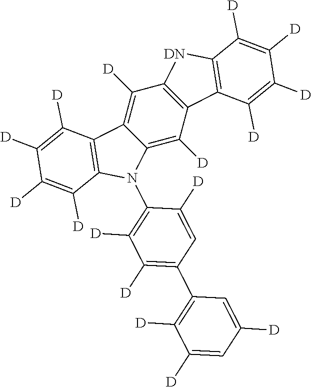 | 90% | 82% |
| 226C | 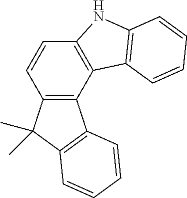 [SMILES: CC1(C)c2ccccc2-c2c1ccc1[nH]c3ccccc3c21] | 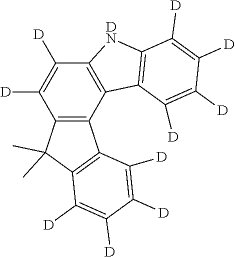 | 91% | 100% |
| 247C | 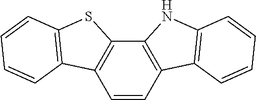 [SMILES: c1ccc2c(c1)[nH]c1c2ccc2c3ccccc3sc21] | 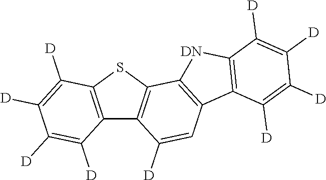 | 88% | 91% |
| 251C | 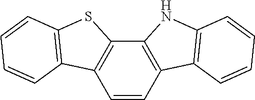 [SMILES: c1ccc2c(c1)[nH]c1c2ccc2c3ccccc3sc21] | 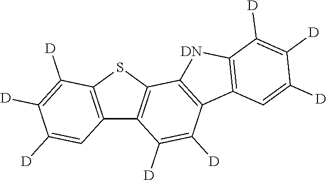 | 90% | 82% |
| 253C | 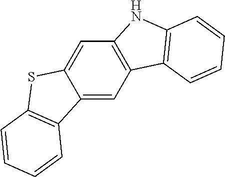 [SMILES: c1ccc2c(c1)[nH]c1cc3sc4ccccc4c3cc12] | 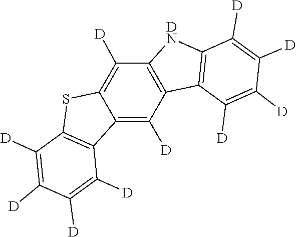 | 95% | 100% |
| 258C | 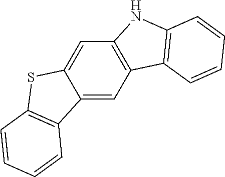 [SMILES: c1ccc2c(c1)[nH]c1cc3sc4ccccc4c3cc12] | 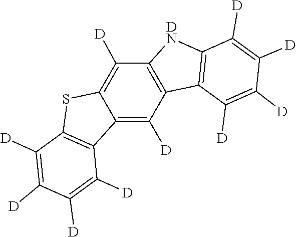 | 96% | 100% |
| 278C | 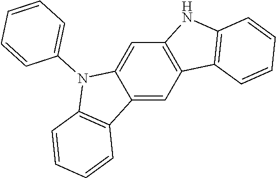 [SMILES: c1ccc(-n2c3ccccc3c3cc4c(cc32)[nH]c2ccccc24)cc1] | 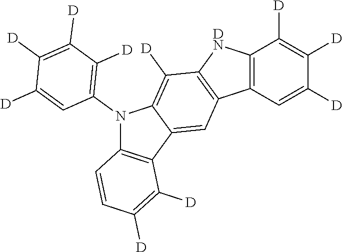 | 90% | 68% |
| 298C | 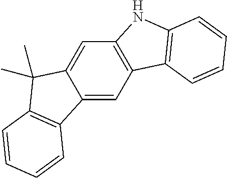 [SMILES: CC1(C)c2ccccc2-c2cc3c(cc21)[nH]c1ccccc13] | 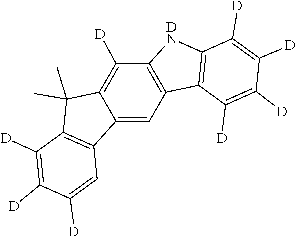 | 80% | 82% |
| 317C | 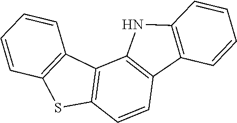 [SMILES: c1ccc2c(c1)[nH]c1c2ccc2sc3ccccc3c21] | 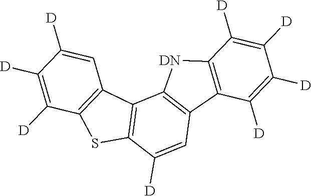 | 91% | 82% |
| 335C | 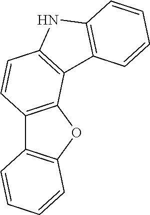 [SMILES: c1ccc2c(c1)[nH]c1ccc3c4ccccc4oc3c12] | 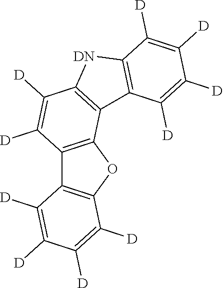 | 96% | 100% |
| 352C | 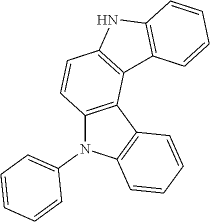 [SMILES: c1ccc(-n2c3ccccc3c3c4c(ccc32)[nH]c2ccccc24)cc1] | 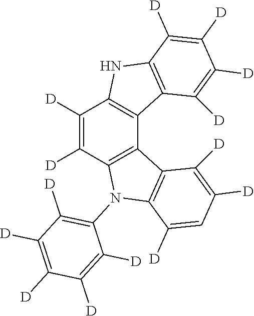 [SMILES: [2H]c1cc([2H])c2c(c1[2H])c1c3c([nH]c4c([2H])c([2H])c([2H])c([2H])c43)c([2H])c([2H])c1n2-c1c([2H])c([2H])c([2H])c([2H])c1[2H]] | 85% | 94% |
| 376C | 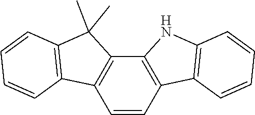 [SMILES: CC1(C)c2ccccc2-c2ccc3c([nH]c4ccccc43)c21] | 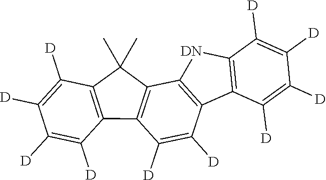 | 89% | 100% |
| 461C | 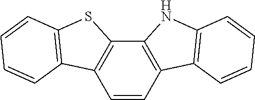 [SMILES: c1ccc2c(c1)[nH]c1c2ccc2c3ccccc3sc21] | 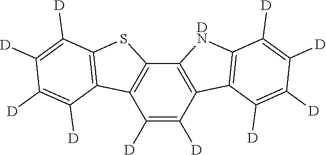 | 80% | 100% |
| 479C | 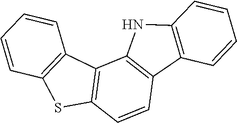 [SMILES: c1ccc2c(c1)[nH]c1c2ccc2sc3ccccc3c21] | 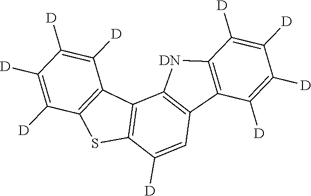 | 77% | 91% |
| 488C | 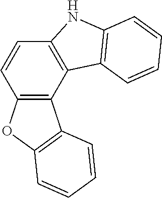 [SMILES: c1ccc2c(c1)[nH]c1ccc3oc4ccccc4c3c12] | 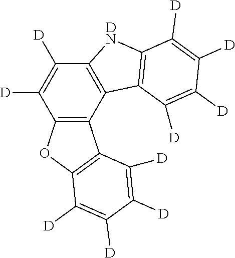 | 89% | 100% |
| 495C | 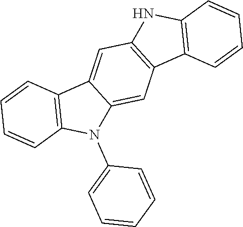 [SMILES: c1ccc(-n2c3ccccc3c3cc4[nH]c5ccccc5c4cc32)cc1] | 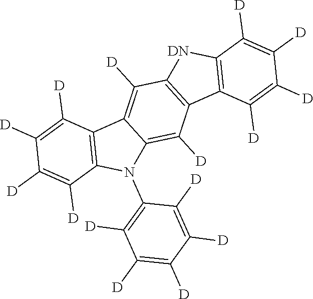 | 74% | 100% |
| 542C | 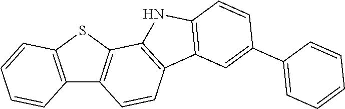 [SMILES: c1ccc(-c2ccc3[nH]c4c(ccc5c6ccccc6sc54)c3c2)cc1] | 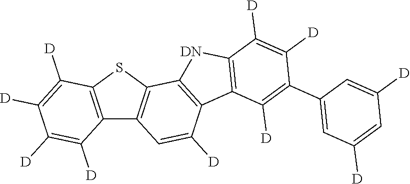 | 88% | 73% |
| 560C | 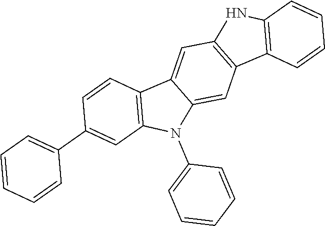 [SMILES: c1ccc(-c2ccc3c4cc5[nH]c6ccccc6c5cc4n(-c4ccccc4)c3c2)cc1] | 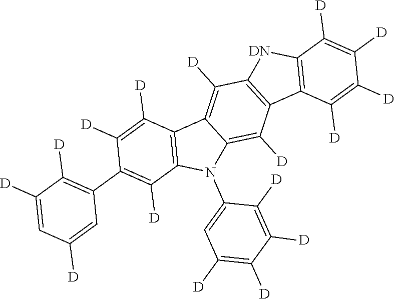 | 79% | 85% |

### Preparation Example 2

### Preparation of Compound 9

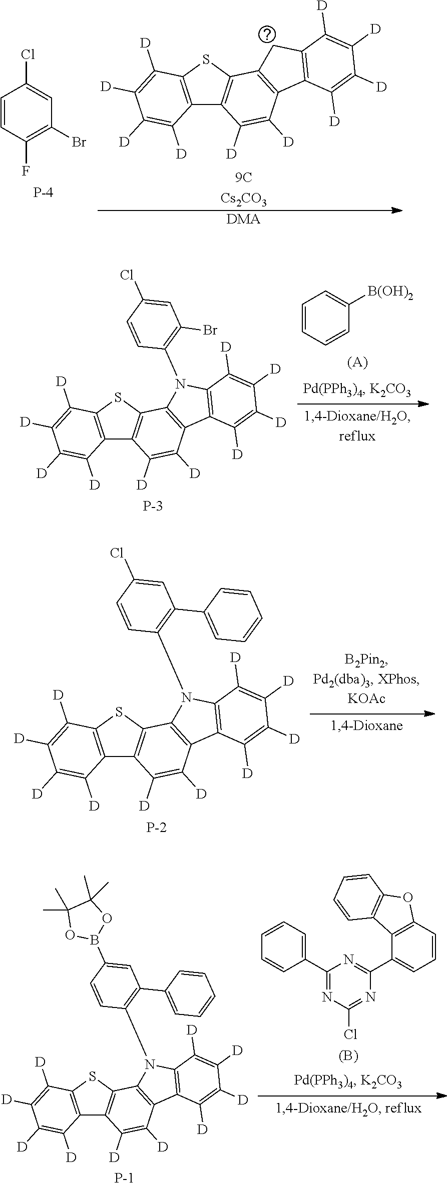

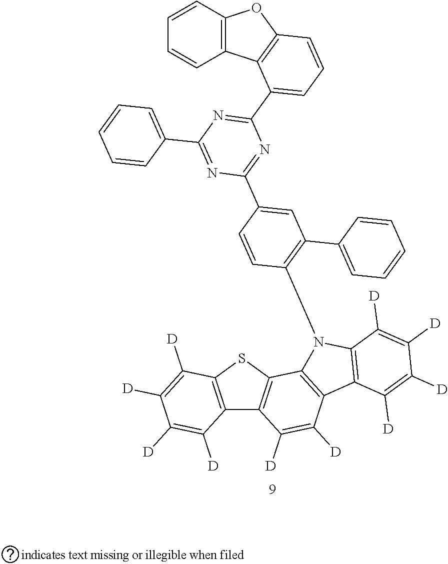 [SMILES: [2H]c1c([2H])c([2H])c2c(sc3c2c([2H])c([2H])c2c4c([2H])c([2H])c([2H])c([2H])c4n(-c4ccc(-c5nc(-c6ccccc6)nc(-c6cccc7oc8ccccc8c67)n5)cc4-c4ccccc4)c32)c1[2H]]

### 1) Preparation of Compound P-3

After Compound P-4 (2-bromo-4-chloro-1-fluorobenzene) (20 g, 95.49 mmol), Compound 9C (27 g, 95.49 mmol), and Ca₂CO₃ (62 g, 190.98 mmol) were dissolved in 300 mL of dimethyl acetamide (DMA), the resulting solution was stirred at a reaction temperature of 150° C. for 8 hours. After the reaction was completed, the solution was cooled to room temperature, and an excess amount of H₂O was added thereto to precipitate a solid. The precipitated solid was filtered, washed with H₂O and methanol (MeOH), and then dried. The dried solid was dissolved in an excess amount of methylene chloride (MC) and then filtered through silica gel. The solvent was removed from the filtrate using a rotary evaporator and the residue was recrystallized with EA to obtain 39 g of Compound P-3 as a pale brown solid with a yield of 87%.

### 2) Preparation of Compound P-2

After Compound P-3 (39 g, 82.48 mmol), phenylboronic acid (A) (10 g, 82.48 mmol), tetrakis(triphenylphosphine) palladium (0) (Pd(PPh₃)₄) (4.7 g, 4.15 mmol), and K₂CO₃ (17 g, 123.72 mmol) were dissolved in 500 mL of 1,4-dioxane/100 mL of H₂O, the resulting solution was stirred at a reaction temperature of 100° C. for 4 hours. After the reaction was completed, the solution was cooled to room temperature, and the solvent was removed using a rotary evaporator. After the concentrated solution was dissolved in an excess amount of MC, extraction was performed with H₂O, and the organic layer was dried over anhydrous MgSO₄ and then filtered through silica gel. The solvent was removed from the filtrate using a rotary evaporator and the residue was recrystallized with EA to obtain 31 g of Compound P-2 as a pale yellow solid with a yield of 79%.

### 3) Preparation of Compound P-1

After Compound P-2 (31 g, 55.26 mmol), B₂Pin₂ (28 g, 110.52 mmol), tris(dibenzylideneacetone)dipalladium(0) (Pd₂(dba)₃) (5.1 g, 5.53 mmol), 2-dicyclohexylphosphino-2′,4′,6′-triisopropylbiphenyl (XPhos) (6.3 g, 11.06 mmol), and potassium acetate (KOAc) (11 g, 110.52 mmol) were dissolved in 500 mL of 1,4-dioxane, the resulting solution was stirred at a reaction temperature of 100° C. for 14 hours. After the reaction was completed, the solution was cooled to room temperature, and the inorganic salts were filtered off. After the filtrate was concentrated using a rotary evaporator, the concentrate was dissolved in an excess amount of MC, and filtered through silica gel. The solvent was removed from the filtrate using a rotary evaporator and the residue was recrystallized with MeOH to obtain 27 g of Compound P-1 as a yellow solid with a yield of 87%.

### 4) Preparation of Compound 9

After Compound P-1 (10 g, 17.81 mmol), 2-chloro-4-(dibenzo[b,d]furan-1-yl)-6-phenyl-1,3,5-triazine (B) (6.4 g, 17.81 mmol), tetrakis(triphenylphosphine) palladium (0) (Pd(PPh₃)₄) (1.1 g, 0.89 mmol), and K₂CO₃ (5 g, 35.62 mmol) were dissolved in 120 mL of 1,4-dioxane/30 mL of H₂O, the resulting solution was stirred at a reaction temperature of 100° C. for 4 hours. After the reaction was completed, the solution was cooled to room temperature, and the solvent was removed using a rotary evaporator. After the concentrated solution was dissolved in an excess amount of MC, extraction was performed with H₂O, and the organic layer was dried over anhydrous MgSO₄ and then filtered through silica gel. The solvent was removed from the filtrate using a rotary evaporator, the residue was recrystallized with chlorobenzene (CB)/hexane (Hex) to obtain 9.2 g of Compound 9 as a pale ivory solid with a yield of 67%.

Final compounds in the following Table 3 were synthesized by performing the preparation in the same manner as in Preparation Example 2, except that Intermediate 2 in the following Table 2 was used instead of Compound P-4, Intermediate 3 in the following Table 2 was used instead of Compound 9C, Intermediate 4 in the following Table 2 was used instead of Compound (A), and Intermediate 5 in the following Table 2 was used instead of Compound (B).

TABLE 2

| Compound | | |
|---|---|---|
| No. | Intermediate 2 | Intermediate 3 |
| 12 | 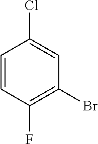 | 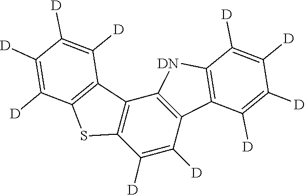 |
| 13 | 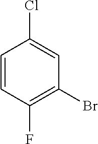 | 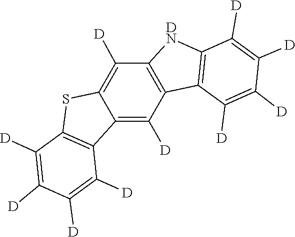 |
| 17 | 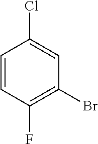 | 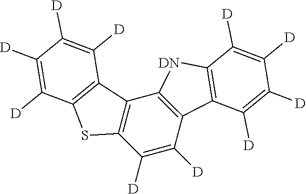 |
| 28 | 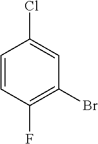 | 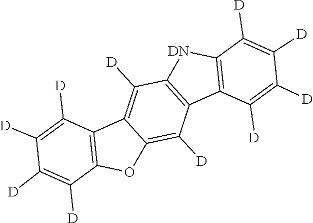 |
| 29 | 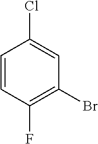 | 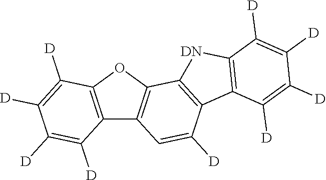 |
| 32 | 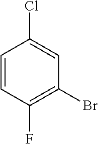 | 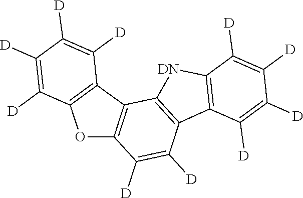 |
| 38 | 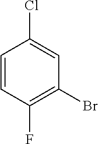 | 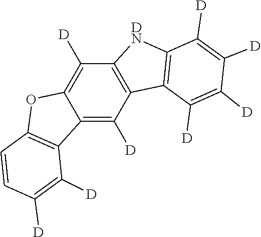 |
| 41 | 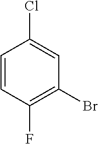 | 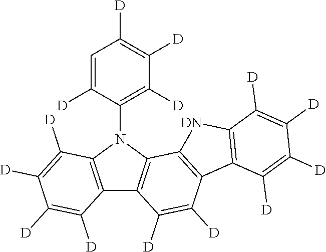 |
| 46 | 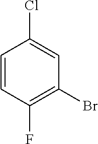 | 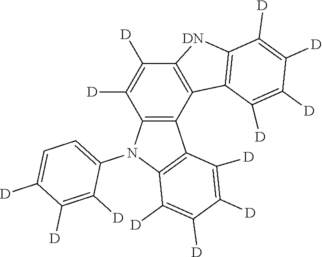 |
| 47 | 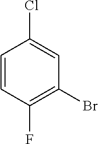 | 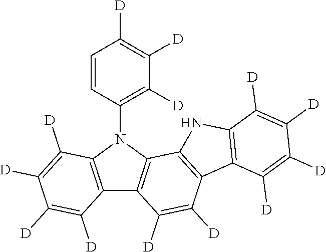 [SMILES: [2H]c1ccc(-n2c3c([2H])c([2H])c([2H])c([2H])c3c3c([2H])c([2H])c4c([nH]c5c([2H])c([2H])c([2H])c([2H])c54)c32)c([2H])c1[2H]] |
| 59 | 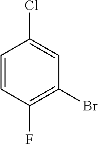 | 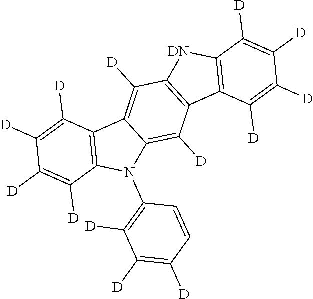 |
| 63 | 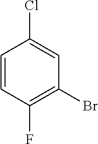 | 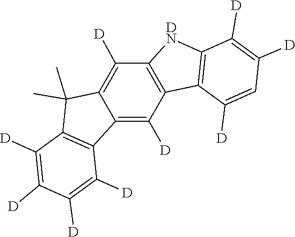 |
| 80 | 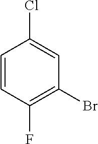 | 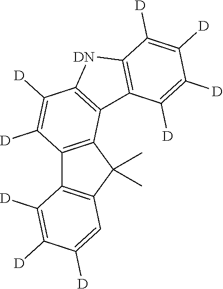 |
| 97 | 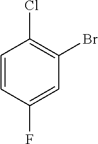 | 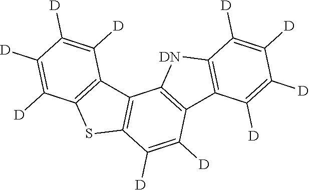 |
| 106 | 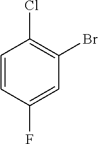 | 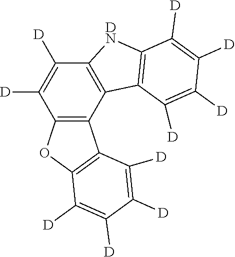 |
| 111 | 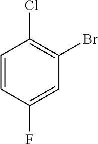 | 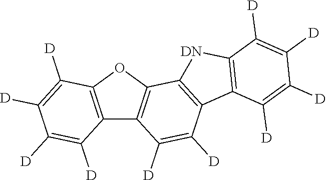 |
| 113 | 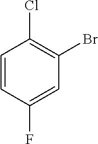 | 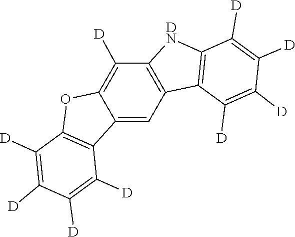 |
| 135 | 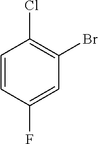 | 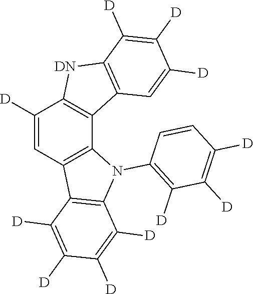 |
| 136 | 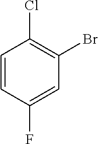 | 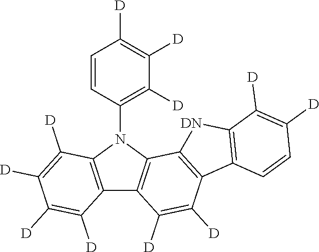 |
| 138 | 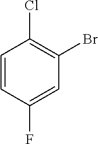 | 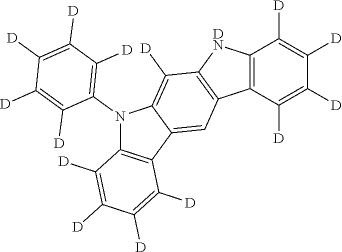 |
| 154 | 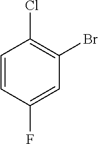 | 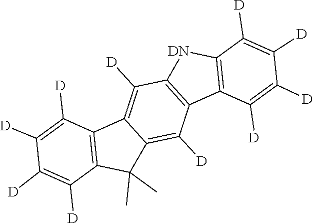 |
| 166 | 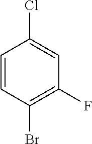 | 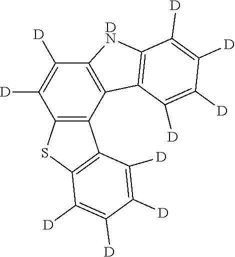 |
| 178 | 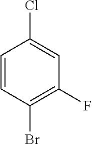 | 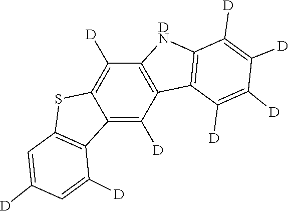 |
| 179 | 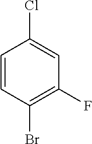 | 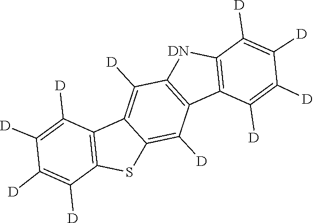 |
| 180 | 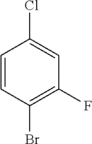 | 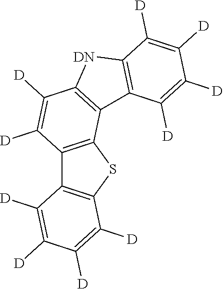 |
| 186 | 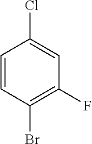 | 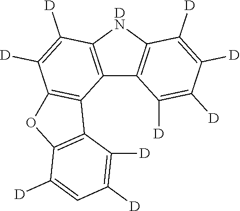 |
| 196 | 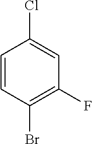 | 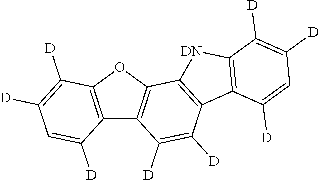 |
| 197 | 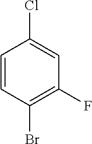 | 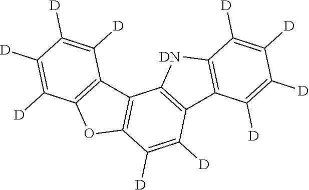 |
| 207 | 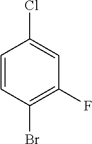 | 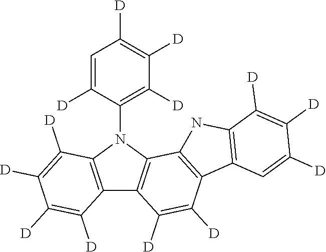 |
| 214 | 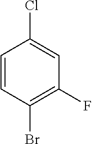 | 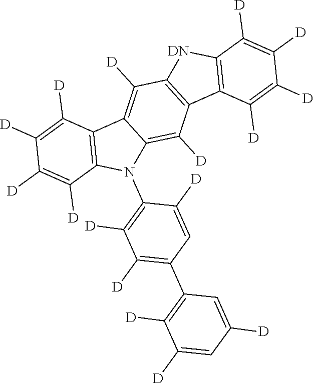 |
| 226 | 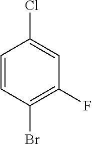 | 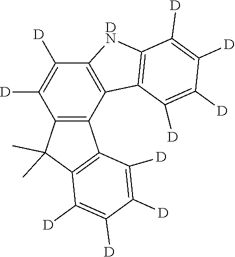 |
| 247 | 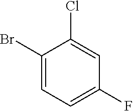 | 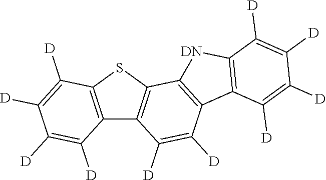 |
| 251 | 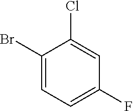 | 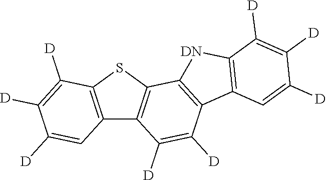 |
| 253 | 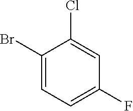 | 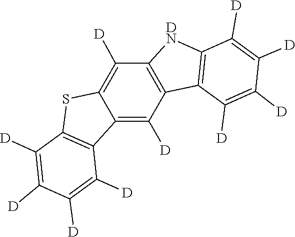 |
| 278 | 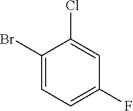 | 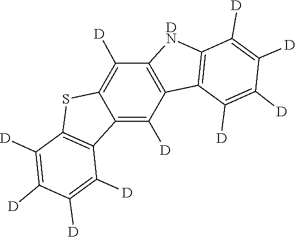 |
| 298 | 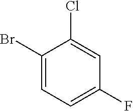 | 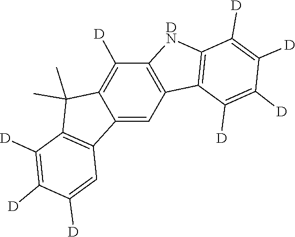 |
| 317 | 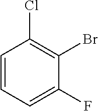 | 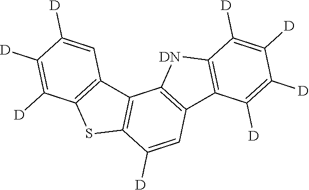 |
| 335 | 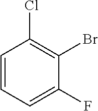 | 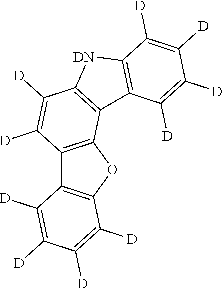 |
| 352 | 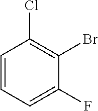 | 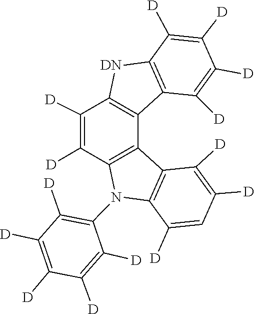 |
| 376 | 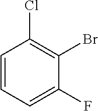 | 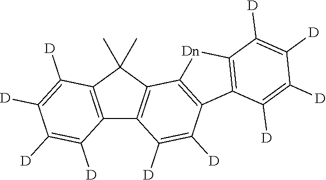 |
| 461 | 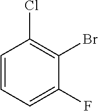 | 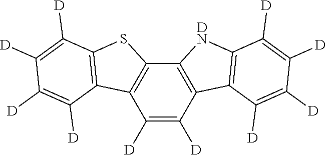 |
| 479 | 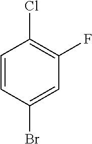 | 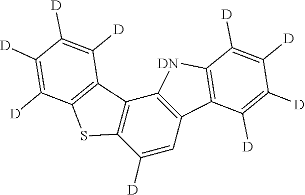 |
| 488 | 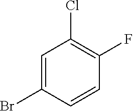 | 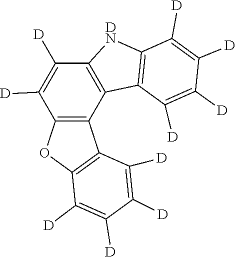 |
| 495 | 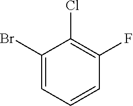 | 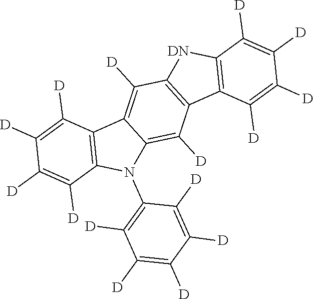 |
| 542 | 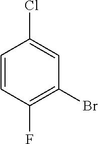 | 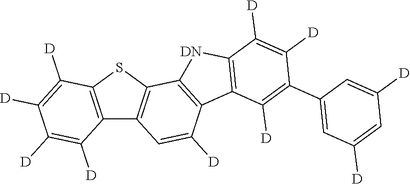 |
| 560 | 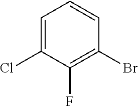 | 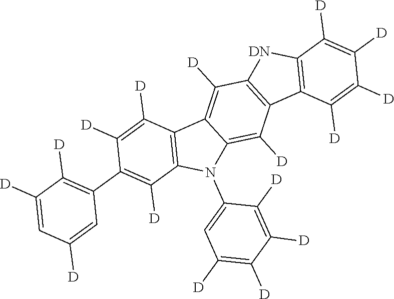 |
| Compound | | |
| No. | Intermediate 4 | Intermediate 5 |
| 12 | 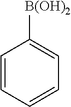 | 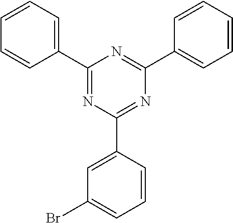 [SMILES: Brc1cccc(-c2nc(-c3ccccc3)nc(-c3ccccc3)n2)c1] |
| 13 | 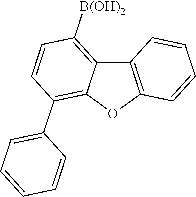 [SMILES: OB(O)c1ccc(-c2ccccc2)c2oc3ccccc3c12] | 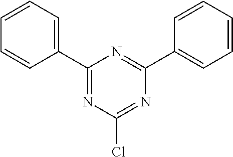 [SMILES: Clc1nc(-c2ccccc2)nc(-c2ccccc2)n1] |
| 17 | 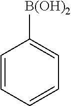 | 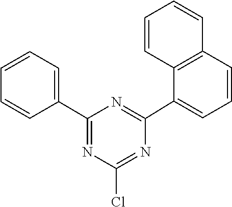 [SMILES: Clc1nc(-c2ccccc2)nc(-c2cccc3ccccc23)n1] |
| 28 | 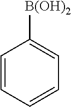 | 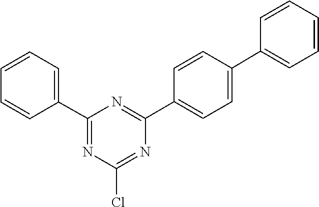 [SMILES: Clc1nc(-c2ccccc2)nc(-c2ccc(-c3ccccc3)cc2)n1] |
| 29 | 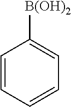 | 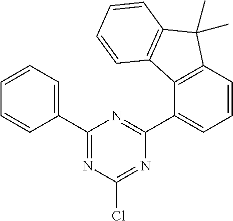 [SMILES: CC1(C)c2ccccc2-c2c(-c3nc(Cl)nc(-c4ccccc4)n3)cccc21] |
| 32 | 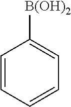 | 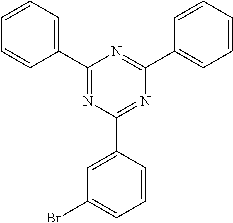 [SMILES: Brc1cccc(-c2nc(-c3ccccc3)nc(-c3ccccc3)n2)c1] |
| 38 | 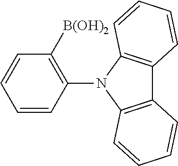 [SMILES: OB(O)c1ccccc1-n1c2ccccc2c2ccccc21] | 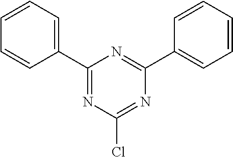 [SMILES: Clc1nc(-c2ccccc2)nc(-c2ccccc2)n1] |
| 41 | 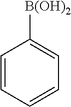 | 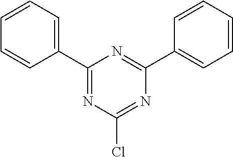 [SMILES: Clc1nc(-c2ccccc2)nc(-c2ccccc2)n1] |
| 46 | 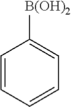 | 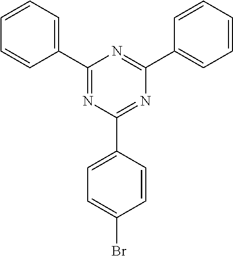 [SMILES: Brc1ccc(-c2nc(-c3ccccc3)nc(-c3ccccc3)n2)cc1] |
| 47 | 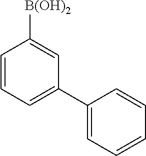 [SMILES: OB(O)c1cccc(-c2ccccc2)c1] | 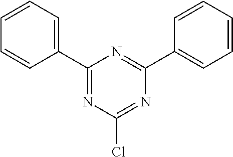 [SMILES: Clc1nc(-c2ccccc2)nc(-c2ccccc2)n1] |
| 59 | 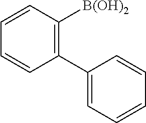 [SMILES: OB(O)c1ccccc1-c1ccccc1] | 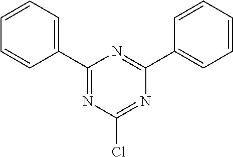 [SMILES: Clc1nc(-c2ccccc2)nc(-c2ccccc2)n1] |
| 63 | 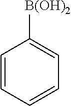 | 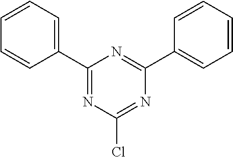 [SMILES: Clc1nc(-c2ccccc2)nc(-c2ccccc2)n1] |
| 80 | 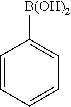 | 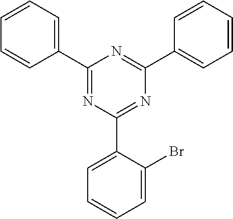 [SMILES: Brc1ccccc1-c1nc(-c2ccccc2)nc(-c2ccccc2)n1] |
| 97 | 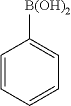 | 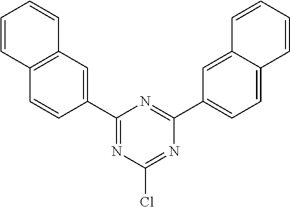 [SMILES: Clc1nc(-c2ccc3ccccc3c2)nc(-c2ccc3ccccc3c2)n1] |
| 106 | 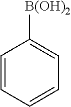 | 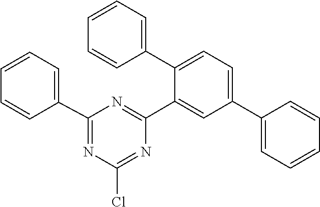 [SMILES: Clc1nc(-c2ccccc2)nc(-c2cc(-c3ccccc3)ccc2-c2ccccc2)n1] |
| 111 | 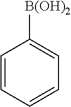 | 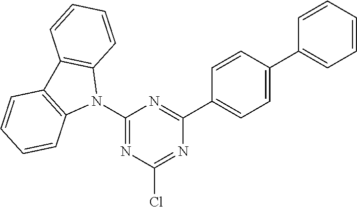 [SMILES: Clc1nc(-c2ccc(-c3ccccc3)cc2)nc(-n2c3ccccc3c3ccccc32)n1] |
| 113 | 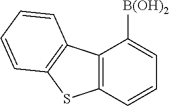 [SMILES: OB(O)c1cccc2sc3ccccc3c12] | 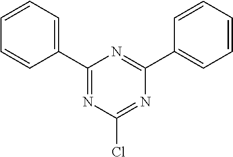 [SMILES: Clc1nc(-c2ccccc2)nc(-c2ccccc2)n1] |
| 135 | 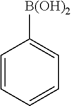 | 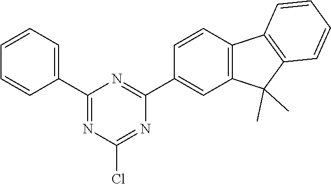 [SMILES: CC1(C)c2ccccc2-c2ccc(-c3nc(Cl)nc(-c4ccccc4)n3)cc21] |
| 136 | 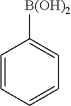 | 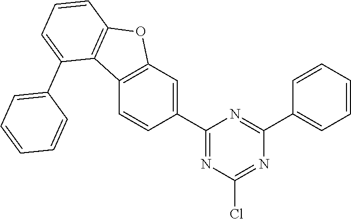 [SMILES: Clc1nc(-c2ccccc2)nc(-c2ccc3c(c2)oc2cccc(-c4ccccc4)c23)n1] |
| 138 | 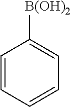 | 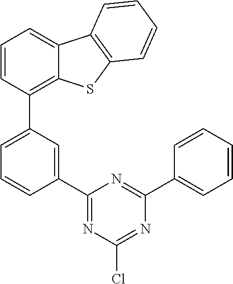 [SMILES: Clc1nc(-c2ccccc2)nc(-c2cccc(-c3cccc4c3sc3ccccc34)c2)n1] |
| 154 | 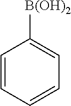 | 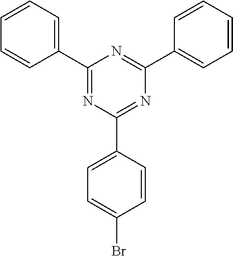 [SMILES: Brc1ccc(-c2nc(-c3ccccc3)nc(-c3ccccc3)n2)cc1] |
| 166 | 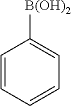 | 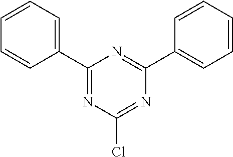 [SMILES: Clc1nc(-c2ccccc2)nc(-c2ccccc2)n1] |
| 178 | 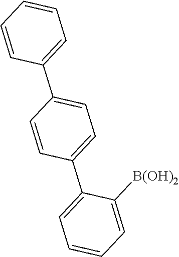 [SMILES: OB(O)c1ccccc1-c1ccc(-c2ccccc2)cc1] | 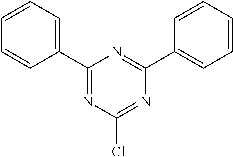 [SMILES: Clc1nc(-c2ccccc2)nc(-c2ccccc2)n1] |
| 179 | 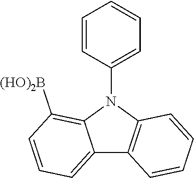 [SMILES: OB(O)c1cccc2c3ccccc3n(-c3ccccc3)c12] | 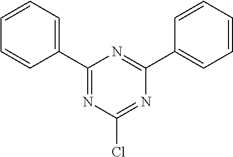 [SMILES: Clc1nc(-c2ccccc2)nc(-c2ccccc2)n1] |
| 180 | 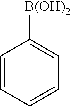 | 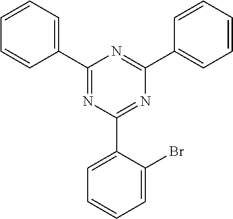 [SMILES: Brc1ccccc1-c1nc(-c2ccccc2)nc(-c2ccccc2)n1] |
| 186 | 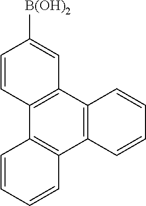 [SMILES: OB(O)c1ccc2c3ccccc3c3ccccc3c2c1] | 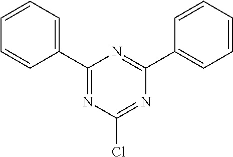 [SMILES: Clc1nc(-c2ccccc2)nc(-c2ccccc2)n1] |
| 196 | 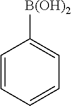 | 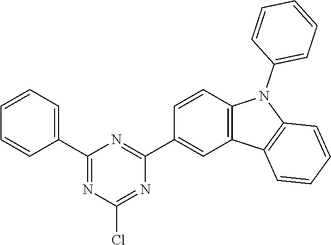 [SMILES: Clc1nc(-c2ccccc2)nc(-c2ccc3c(c2)c2ccccc2n3-c2ccccc2)n1] |
| 197 | 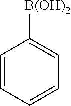 | 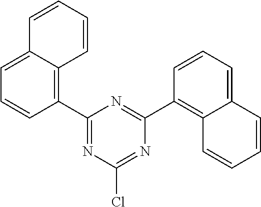 [SMILES: Clc1nc(-c2cccc3ccccc23)nc(-c2cccc3ccccc23)n1] |
| 207 | 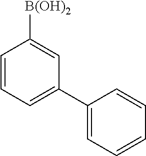 [SMILES: OB(O)c1cccc(-c2ccccc2)c1] | 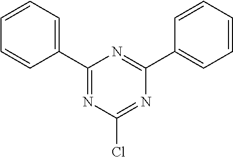 [SMILES: Clc1nc(-c2ccccc2)nc(-c2ccccc2)n1] |
| 214 | 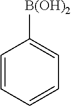 | 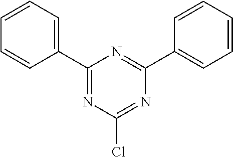 [SMILES: Clc1nc(-c2ccccc2)nc(-c2ccccc2)n1] |
| 226 | 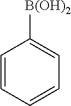 | 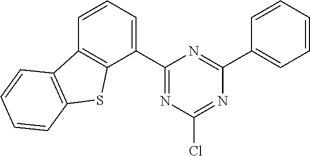 [SMILES: Clc1nc(-c2ccccc2)nc(-c2cccc3c2sc2ccccc23)n1] |
| 247 | 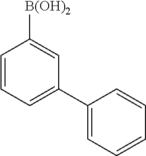 [SMILES: OB(O)c1cccc(-c2ccccc2)c1] | 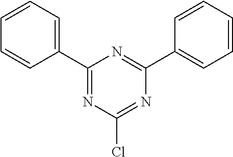 [SMILES: Clc1nc(-c2ccccc2)nc(-c2ccccc2)n1] |
| 251 | 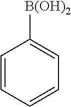 | 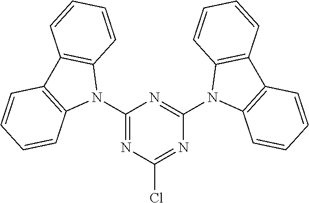 [SMILES: Clc1nc(-n2c3ccccc3c3ccccc32)nc(-n2c3ccccc3c3ccccc32)n1] |
| 253 | 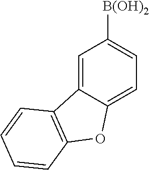 [SMILES: OB(O)c1ccc2oc3ccccc3c2c1] | 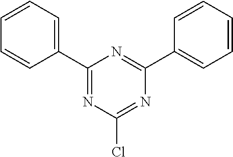 [SMILES: Clc1nc(-c2ccccc2)nc(-c2ccccc2)n1] |
| 278 | 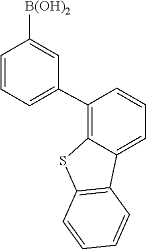 [SMILES: OB(O)c1cccc(-c2cccc3c2sc2ccccc23)c1] | 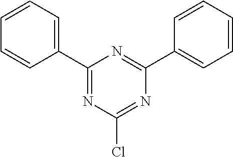 [SMILES: Clc1nc(-c2ccccc2)nc(-c2ccccc2)n1] |
| 298 | 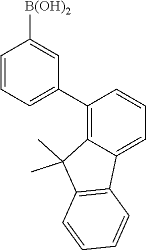 [SMILES: CC1(C)c2ccccc2-c2cccc(-c3cccc(B(O)O)c3)c21] | 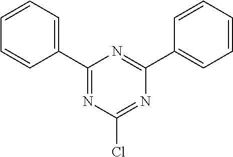 [SMILES: Clc1nc(-c2ccccc2)nc(-c2ccccc2)n1] |
| 317 | 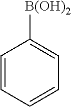 | 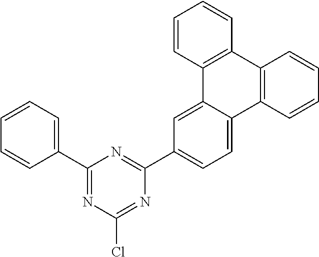 [SMILES: Clc1nc(-c2ccccc2)nc(-c2ccc3c4ccccc4c4ccccc4c3c2)n1] |
| 335 | 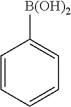 | 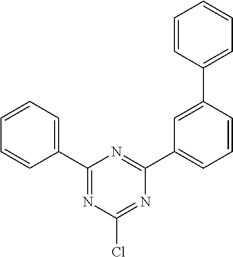 [SMILES: Clc1nc(-c2ccccc2)nc(-c2cccc(-c3ccccc3)c2)n1] |
| 352 | 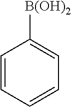 | 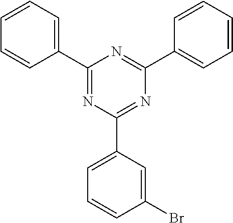 [SMILES: Brc1cccc(-c2nc(-c3ccccc3)nc(-c3ccccc3)n2)c1] |
| 376 | 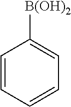 | 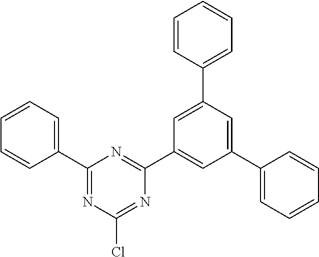 [SMILES: Clc1nc(-c2ccccc2)nc(-c2cc(-c3ccccc3)cc(-c3ccccc3)c2)n1] |
| 461 | 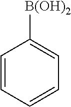 | 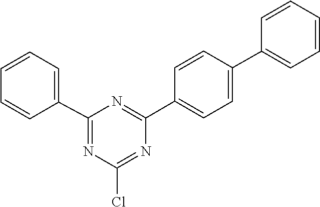 [SMILES: Clc1nc(-c2ccccc2)nc(-c2ccc(-c3ccccc3)cc2)n1] |
| 479 | 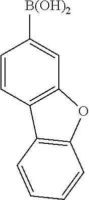 [SMILES: OB(O)c1ccc2c(c1)oc1ccccc12] | 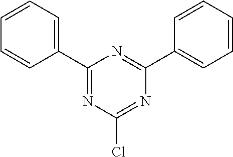 [SMILES: Clc1nc(-c2ccccc2)nc(-c2ccccc2)n1] |
| 488 | 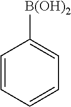 | 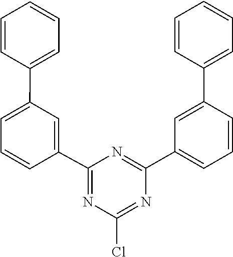 [SMILES: Clc1nc(-c2cccc(-c3ccccc3)c2)nc(-c2cccc(-c3ccccc3)c2)n1] |
| 495 | 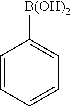 | 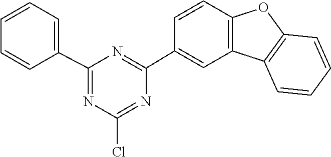 [SMILES: Clc1nc(-c2ccccc2)nc(-c2ccc3oc4ccccc4c3c2)n1] |
| 542 | 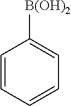 | 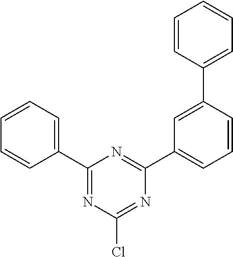 [SMILES: Clc1nc(-c2ccccc2)nc(-c2cccc(-c3ccccc3)c2)n1] |
| 560 | 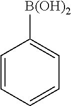 | 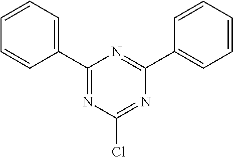 [SMILES: Clc1nc(-c2ccccc2)nc(-c2ccccc2)n1] |

TABLE 3

| Compound | | | D |
|---|---|---|---|
| No. | Final compound | Yield | substitution rate |
| 12 | 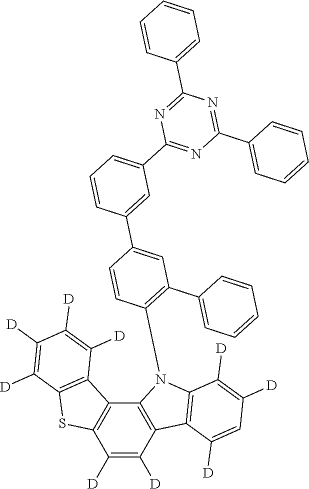 [SMILES: [2H]c1cc([2H])c2c3c([2H])c([2H])c4sc5c([2H])c([2H])c([2H])c([2H])c5c4c3n(-c3ccc(-c4cccc(-c5nc(-c6ccccc6)nc(-c6ccccc6)n5)c4)cc3-c3ccccc3)c2c1[2H]] | 55% | 29% |
| 13 | 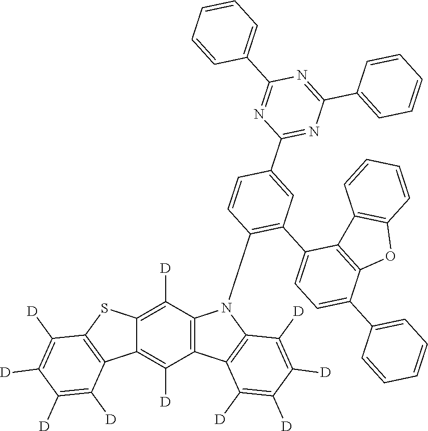 [SMILES: [2H]c1c([2H])c([2H])c2c(sc3c([2H])c4c(c([2H])c32)c2c([2H])c([2H])c([2H])c([2H])c2n4-c2ccc(-c3nc(-c4ccccc4)nc(-c4ccccc4)n3)cc2-c2ccc(-c3ccccc3)c3oc4ccccc4c23)c1[2H]] | 62% | 29% |
| 17 | 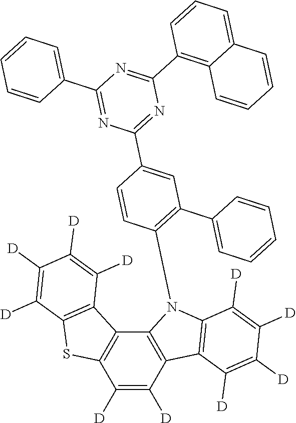 [SMILES: [2H]c1c([2H])c([2H])c2c(sc3c([2H])c([2H])c4c5c([2H])c([2H])c([2H])c([2H])c5n(-c5ccc(-c6nc(-c7ccccc7)nc(-c7cccc8ccccc78)n6)cc5-c5ccccc5)c4c32)c1[2H]] | 65% | 33% |
| 28 | 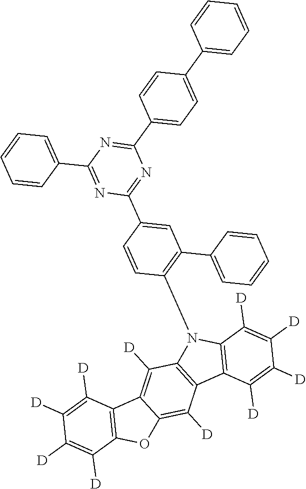 [SMILES: [2H]c1c([2H])c([2H])c2c(oc3c([2H])c4c5c([2H])c([2H])c([2H])c([2H])c5n(-c5ccc(-c6nc(-c7ccccc7)nc(-c7ccc(-c8ccccc8)cc7)n6)cc5-c5ccccc5)c4c([2H])c32)c1[2H]] | 55% | 31% |
| 29 | 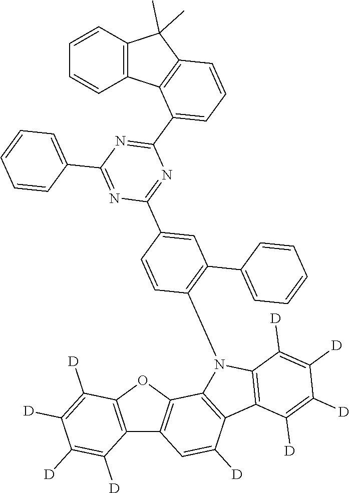 [SMILES: [2H]c1c([2H])c([2H])c2c(oc3c2cc([2H])c2c4c([2H])c([2H])c([2H])c([2H])c4n(-c4ccc(-c5nc(-c6ccccc6)nc(-c6cccc7c6-c6ccccc6C7(C)C)n5)cc4-c4ccccc4)c32)c1[2H]] | 50% | 25% |
| 32 | 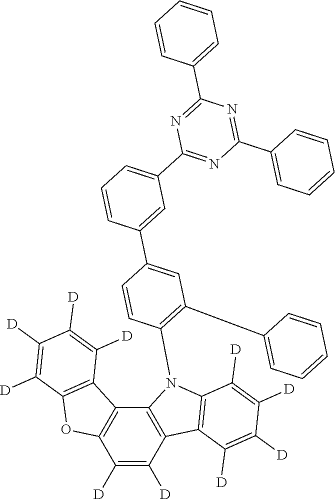 [SMILES: [2H]c1c([2H])c([2H])c2c(oc3c([2H])c([2H])c4c5c([2H])c([2H])c([2H])c([2H])c5n(-c5ccc(-c6cccc(-c7nc(-c8ccccc8)nc(-c8ccccc8)n7)c6)cc5-c5ccccc5)c4c32)c1[2H]] | 62% | 31% |
| 38 | 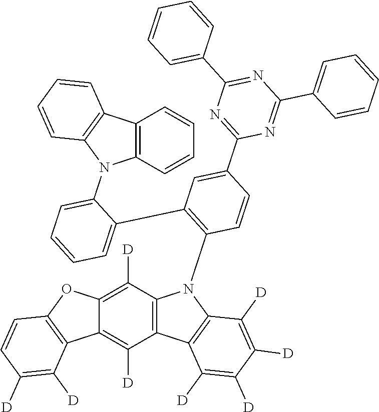 [SMILES: [2H]c1ccc2oc3c([2H])c4c(c([2H])c3c2c1[2H])c1c([2H])c([2H])c([2H])c([2H])c1n4-c1ccc(-c2nc(-c3ccccc3)nc(-c3ccccc3)n2)cc1-c1ccccc1-n1c2ccccc2c2ccccc21] | 66% | 23% |
| 41 | 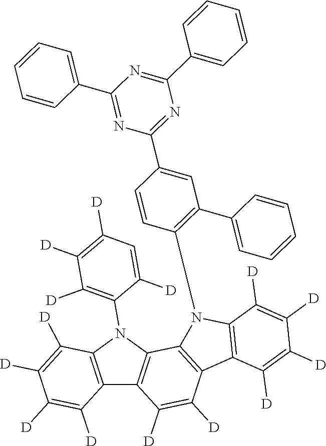 [SMILES: [2H]c1cc([2H])c(-n2c3c([2H])c([2H])c([2H])c([2H])c3c3c([2H])c([2H])c4c5c([2H])c([2H])c([2H])c([2H])c5n(-c5ccc(-c6nc(-c7ccccc7)nc(-c7ccccc7)n6)cc5-c5ccccc5)c4c32)c([2H])c1[2H]] | 46% | 42% |
| 46 | 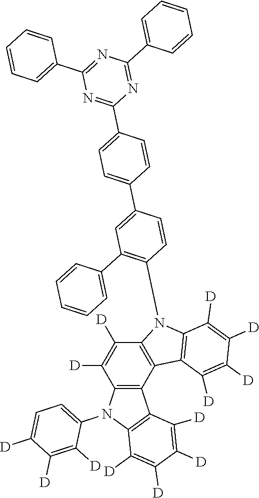 [SMILES: [2H]c1ccc(-n2c3c([2H])c([2H])c([2H])c([2H])c3c3c4c5c([2H])c([2H])c([2H])c([2H])c5n(-c5ccc(-c6ccc(-c7nc(-c8ccccc8)nc(-c8ccccc8)n7)cc6)cc5-c5ccccc5)c4c([2H])c([2H])c32)c([2H])c1[2H]] | 70% | 35% |
| 47 | 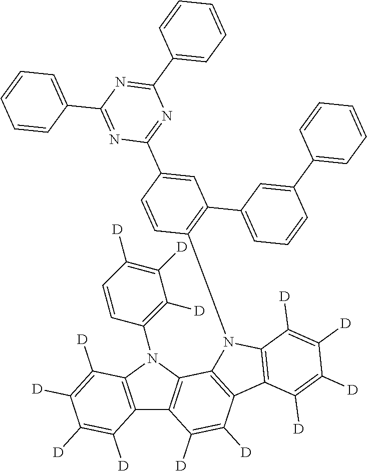 [SMILES: [2H]c1ccc(-n2c3c([2H])c([2H])c([2H])c([2H])c3c3c([2H])c([2H])c4c5c([2H])c([2H])c([2H])c([2H])c5n(-c5ccc(-c6nc(-c7ccccc7)nc(-c7ccccc7)n6)cc5-c5cccc(-c6ccccc6)c5)c4c32)c([2H])c1[2H]] | 68% | 35% |
| 59 | 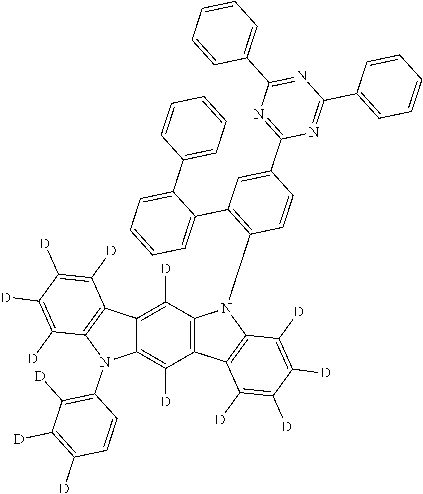 [SMILES: [2H]c1ccc(-n2c3c([2H])c([2H])c([2H])c([2H])c3c3c([2H])c4c(c([2H])c32)c2c([2H])c([2H])c([2H])c([2H])c2n4-c2ccc(-c3nc(-c4ccccc4)nc(-c4ccccc4)n3)cc2-c2ccccc2-c2ccccc2)c([2H])c1[2H]] | 65% | 35% |
| 63 | 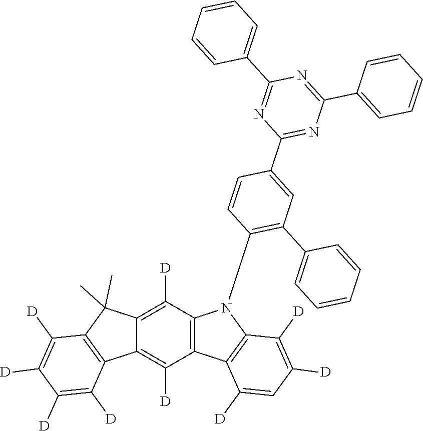 [SMILES: [2H]c1cc([2H])c2c3c([2H])c4c(c([2H])c3n(-c3ccc(-c5nc(-c6ccccc6)nc(-c6ccccc6)n5)cc3-c3ccccc3)c2c1[2H])C(C)(C)c1c([2H])c([2H])c([2H])c([2H])c1-4] | 52% | 26% |
| 80 | 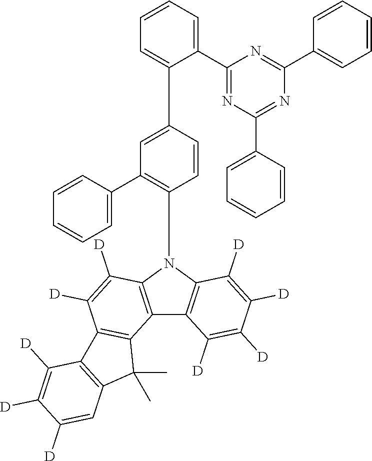 [SMILES: [2H]c1cc2c(c([2H])c1[2H])-c1c([2H])c([2H])c3c(c1C2(C)C)c1c([2H])c([2H])c([2H])c([2H])c1n3-c1ccc(-c2ccccc2-c2nc(-c3ccccc3)nc(-c3ccccc3)n2)cc1-c1ccccc1] | 60% | 24% |
| 97 | 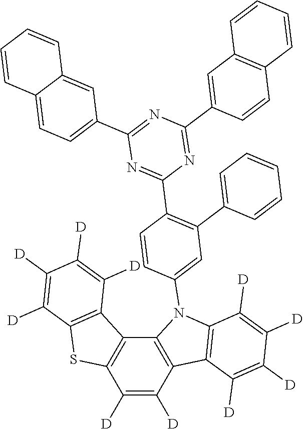 [SMILES: [2H]c1c([2H])c([2H])c2c(sc3c([2H])c([2H])c4c5c([2H])c([2H])c([2H])c([2H])c5n(-c5ccc(-c6nc(-c7ccc8ccccc8c7)nc(-c7ccc8ccccc8c7)n6)c(-c6ccccc6)c5)c4c32)c1[2H]] | 52% | 31% |
| 106 | 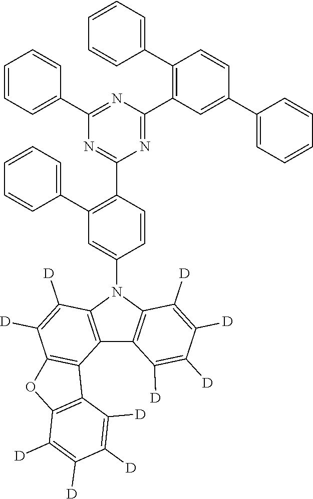 [SMILES: [2H]c1c([2H])c([2H])c2c(oc3c([2H])c([2H])c4c(c5c([2H])c([2H])c([2H])c([2H])c5n4-c4ccc(-c5nc(-c6ccccc6)nc(-c6cc(-c7ccccc7)ccc6-c6ccccc6)n5)c(-c5ccccc5)c4)c32)c1[2H]] | 48% | 28% |
| 111 | 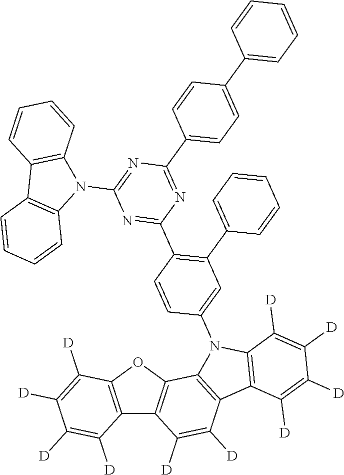 [SMILES: [2H]c1c([2H])c([2H])c2c(oc3c2c([2H])c([2H])c2c4c([2H])c([2H])c([2H])c([2H])c4n(-c4ccc(-c5nc(-c6ccc(-c7ccccc7)cc6)nc(-n6c7ccccc7c7ccccc76)n5)c(-c5ccccc5)c4)c32)c1[2H]] | 67% | 29% |
| 113 | 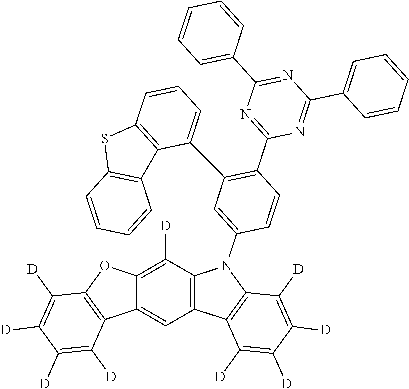 [SMILES: [2H]c1c([2H])c([2H])c2c(oc3c([2H])c4c(cc32)c2c([2H])c([2H])c([2H])c([2H])c2n4-c2ccc(-c3nc(-c4ccccc4)nc(-c4ccccc4)n3)c(-c3cccc4sc5ccccc5c34)c2)c1[2H]] | 55% | 30% |
| 135 | 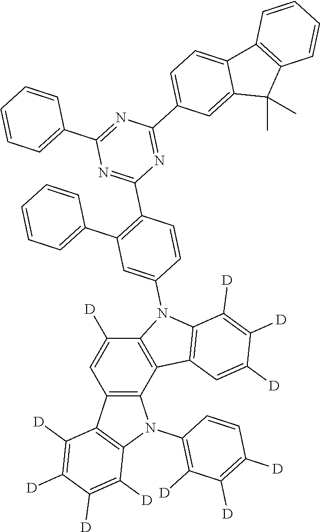 [SMILES: [2H]c1ccc(-n2c3c([2H])c([2H])c([2H])c([2H])c3c3cc([2H])c4c(c5cc([2H])c([2H])c([2H])c5n4-c4ccc(-c5nc(-c6ccccc6)nc(-c6ccc7c(c6)C(C)(C)c6ccccc6-7)n5)c(-c5ccccc5)c4)c32)c([2H])c1[2H]] | 60% | 27% |
| 136 | 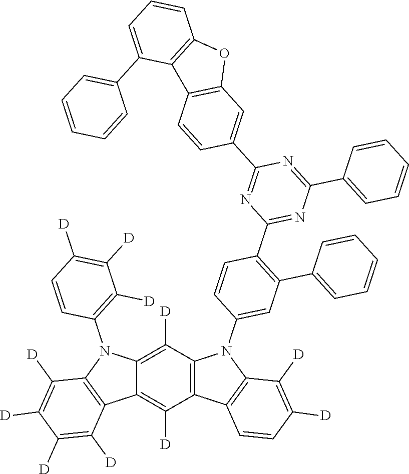 [SMILES: [2H]c1ccc(-n2c3c([2H])c([2H])c([2H])c([2H])c3c3c([2H])c4c5ccc([2H])c([2H])c5n(-c5ccc(-c6nc(-c7ccccc7)nc(-c7ccc8c(c7)oc7cccc(-c9ccccc9)c78)n6)c(-c6ccccc6)c5)c4c([2H])c32)c([2H])c1[2H]] | 66% | 28% |
| 138 | 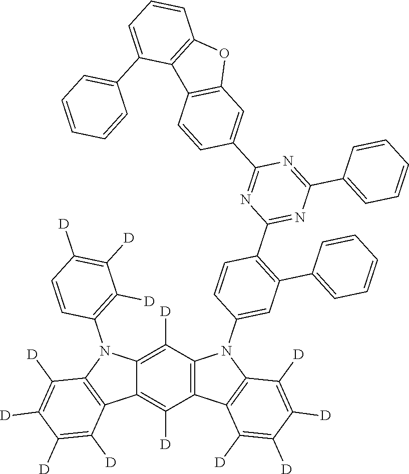 [SMILES: [2H]c1ccc(-n2c3c([2H])c([2H])c([2H])c([2H])c3c3c([2H])c4c5c([2H])c([2H])c([2H])c([2H])c5n(-c5ccc(-c6nc(-c7ccccc7)nc(-c7ccc8c(c7)oc7cccc(-c9ccccc9)c78)n6)c(-c6ccccc6)c5)c4c([2H])c32)c([2H])c1[2H]] | 56% | 36% |
| 154 | 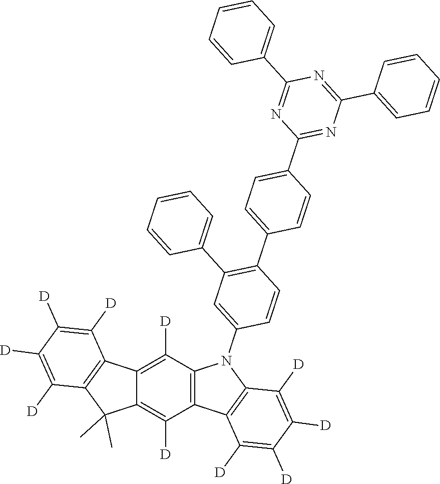 [SMILES: [2H]c1c([2H])c([2H])c2c(c1[2H])-c1c(c([2H])c3c4c([2H])c([2H])c([2H])c([2H])c4n(-c4ccc(-c5ccc(-c6nc(-c7ccccc7)nc(-c7ccccc7)n6)cc5)c(-c5ccccc5)c4)c3c1[2H])C2(C)C] | 52% | 26% |
| 166 | 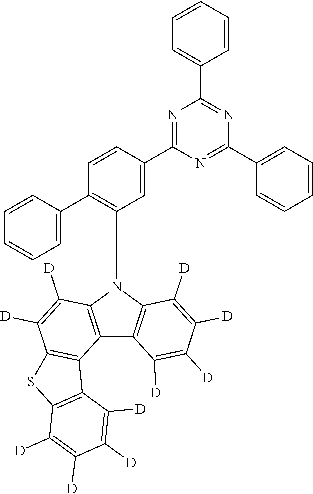 [SMILES: [2H]c1c([2H])c([2H])c2c(sc3c([2H])c([2H])c4c(c5c([2H])c([2H])c([2H])c([2H])c5n4-c4cc(-c5nc(-c6ccccc6)nc(-c6ccccc6)n5)ccc4-c4ccccc4)c32)c1[2H]] | 42% | 36% |
| 178 | 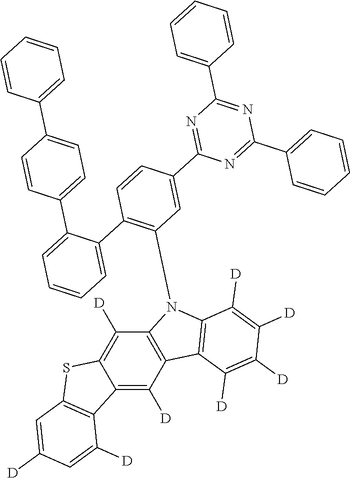 [SMILES: [2H]c1cc([2H])c2c(c1)sc1c([2H])c3c(c([2H])c12)c1c([2H])c([2H])c([2H])c([2H])c1n3-c1cc(-c2nc(-c3ccccc3)nc(-c3ccccc3)n2)ccc1-c1ccccc1-c1ccc(-c2ccccc2)cc1] | 46% | 22% |
| 179 | 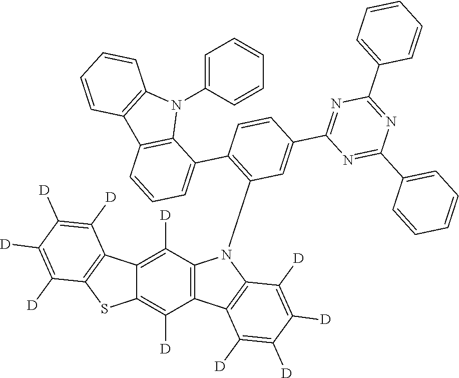 [SMILES: [2H]c1c([2H])c([2H])c2c(sc3c([2H])c4c5c([2H])c([2H])c([2H])c([2H])c5n(-c5cc(-c6nc(-c7ccccc7)nc(-c7ccccc7)n6)ccc5-c5cccc6c7ccccc7n(-c7ccccc7)c56)c4c([2H])c32)c1[2H]] | 70% | 29% |
| 180 | 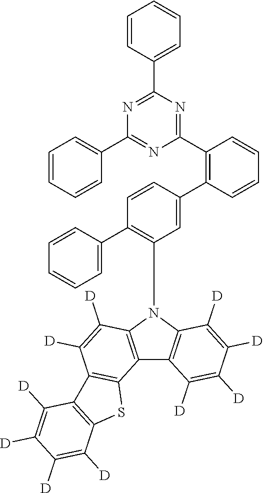 [SMILES: [2H]c1c([2H])c([2H])c2c(sc3c2c([2H])c([2H])c2c3c3c([2H])c([2H])c([2H])c([2H])c3n2-c2cc(-c3ccccc3-c3nc(-c4ccccc4)nc(-c4ccccc4)n3)ccc2-c2ccccc2)c1[2H]] | 68% | 31% |
| 186 | 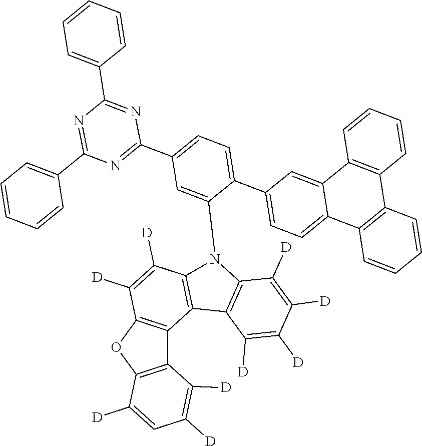 [SMILES: [2H]c1cc([2H])c2oc3c([2H])c([2H])c4c(c5c([2H])c([2H])c([2H])c([2H])c5n4-c4cc(-c5nc(-c6ccccc6)nc(-c6ccccc6)n5)ccc4-c4ccc5c6ccccc6c6ccccc6c5c4)c3c2c1[2H]] | 62% | 26% |
| 196 | 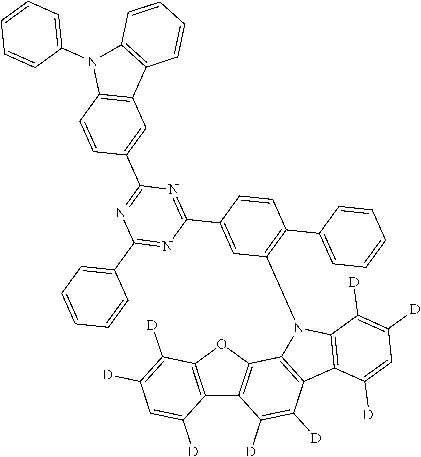 [SMILES: [2H]c1cc([2H])c2c(oc3c2c([2H])c([2H])c2c4c([2H])cc([2H])c([2H])c4n(-c4cc(-c5nc(-c6ccccc6)nc(-c6ccc7c(c6)c6ccccc6n7-c6ccccc6)n5)ccc4-c4ccccc4)c32)c1[2H]] | 49% | 23% |
| 197 | 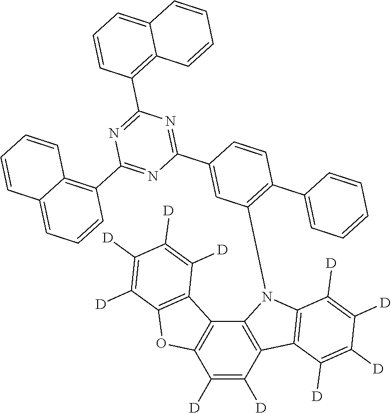 [SMILES: [2H]c1c([2H])c([2H])c2c(oc3c([2H])c([2H])c4c5c([2H])c([2H])c([2H])c([2H])c5n(-c5cc(-c6nc(-c7cccc8ccccc78)nc(-c7cccc8ccccc78)n6)ccc5-c5ccccc5)c4c32)c1[2H]] | 50% | 31% |
| 207 | 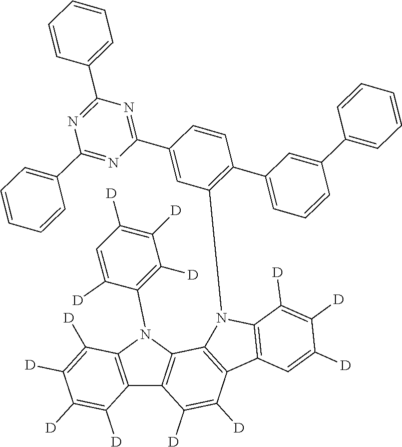 [SMILES: [2H]c1cc([2H])c(-n2c3c([2H])c([2H])c([2H])c([2H])c3c3c([2H])c([2H])c4c5cc([2H])c([2H])c([2H])c5n(-c5cc(-c6nc(-c7ccccc7)nc(-c7ccccc7)n6)ccc5-c5cccc(-c6ccccc6)c5)c4c32)c([2H])c1[2H]] | 50% | 35% |
| 214 | 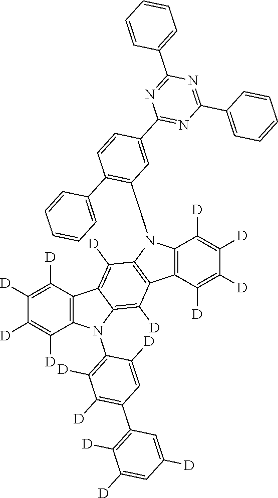 [SMILES: [2H]c1cc([2H])c([2H])c(-c2cc([2H])c(-n3c4c([2H])c([2H])c([2H])c([2H])c4c4c([2H])c5c(c([2H])c43)c3c([2H])c([2H])c([2H])c([2H])c3n5-c3cc(-c4nc(-c5ccccc5)nc(-c5ccccc5)n4)ccc3-c3ccccc3)c([2H])c2[2H])c1] | 46% | 43% |
| 226 | 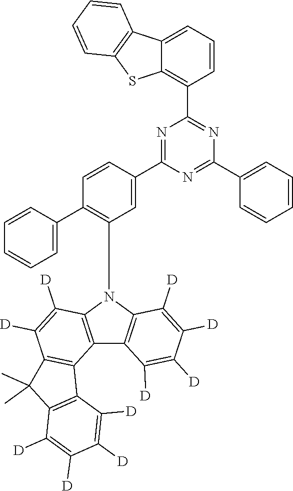 [SMILES: [2H]c1c([2H])c([2H])c2c(c1[2H])-c1c(c([2H])c([2H])c3c1c1c([2H])c([2H])c([2H])c([2H])c1n3-c1cc(-c3nc(-c4ccccc4)nc(-c4cccc5c4sc4ccccc45)n3)ccc1-c1ccccc1)C2(C)C] | 63% | 28% |
| 247 | 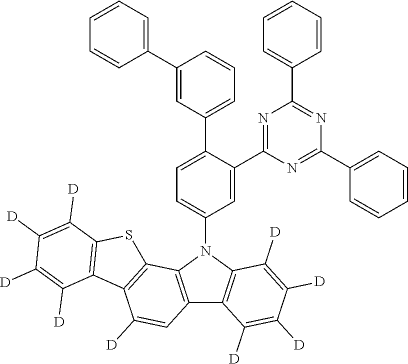 [SMILES: [2H]c1c([2H])c([2H])c2c(sc3c2c([2H])cc2c4c([2H])c([2H])c([2H])c([2H])c4n(-c4ccc(-c5cccc(-c6ccccc6)c5)c(-c5nc(-c6ccccc6)nc(-c6ccccc6)n5)c4)c23)c1[2H]] | 55% | 28% |
| 251 | 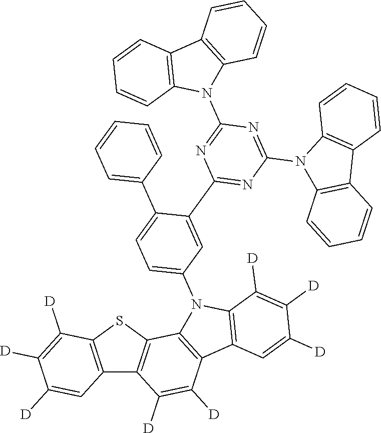 [SMILES: [2H]c1cc2c(sc3c2c([2H])c([2H])c2c4cc([2H])c([2H])c([2H])c4n(-c4ccc(-c5ccccc5)c(-c5nc(-n6c7ccccc7c7ccccc76)nc(-n6c7ccccc7c7ccccc76)n5)c4)c32)c([2H])c1[2H]] | 60% | 24% |
| 253 | 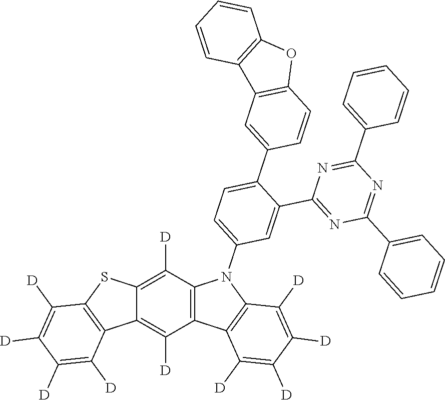 [SMILES: [2H]c1c([2H])c([2H])c2c(sc3c([2H])c4c(c([2H])c32)c2c([2H])c([2H])c([2H])c([2H])c2n4-c2ccc(-c3ccc4oc5ccccc5c4c3)c(-c3nc(-c4ccccc4)nc(-c4ccccc4)n3)c2)c1[2H]] | 70% | 33% |
| 278 | 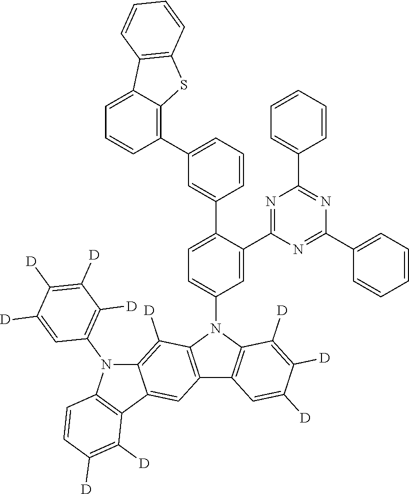 [SMILES: [2H]c1cc(-n2c3ccc([2H])c([2H])c3c3cc4c5cc([2H])c([2H])c([2H])c5n(-c5ccc(-c6cccc(-c7cccc8c7sc7ccccc78)c6)c(-c6nc(-c7ccccc7)nc(-c7ccccc7)n6)c5)c4c([2H])c32)c([2H])c([2H])c1[2H]] | 72% | 26% |
| 298 | 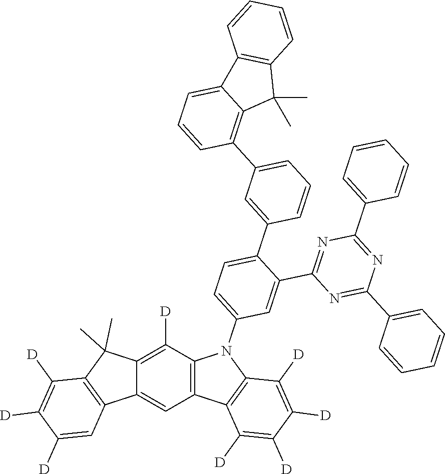 [SMILES: [2H]c1cc2c(c([2H])c1[2H])C(C)(C)c1c-2cc2c3c([2H])c([2H])c([2H])c([2H])c3n(-c3ccc(-c4cccc(-c5cccc6c5C(C)(C)c5ccccc5-6)c4)c(-c4nc(-c5ccccc5)nc(-c5ccccc5)n4)c3)c2c1[2H]] | 55% | 17% |
| 317 | 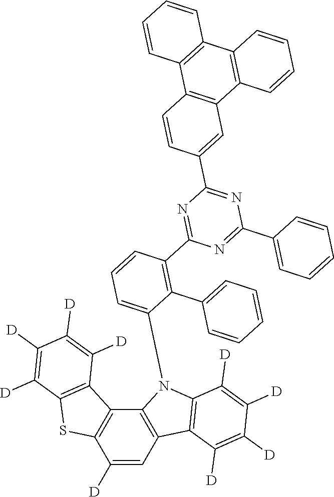 [SMILES: [2H]c1c([2H])c([2H])c2c(sc3c([2H])cc4c5c([2H])c([2H])c([2H])c([2H])c5n(-c5cccc(-c6nc(-c7ccccc7)nc(-c7ccc8c9ccccc9c9ccccc9c8c7)n6)c5-c5ccccc5)c4c32)c1[2H]] | 47% | 26% |
| 335 | 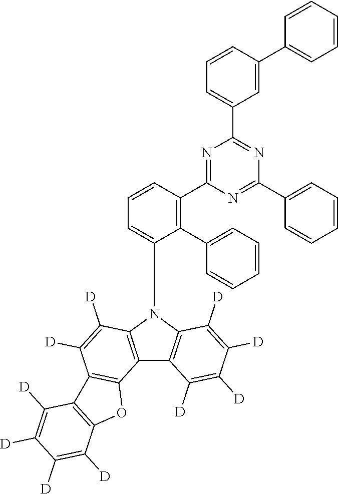 [SMILES: [2H]c1c([2H])c([2H])c2c(oc3c2c([2H])c([2H])c2c3c3c([2H])c([2H])c([2H])c([2H])c3n2-c2cccc(-c3nc(-c4ccccc4)nc(-c4cccc(-c5ccccc5)c4)n3)c2-c2ccccc2)c1[2H]] | 45% | 31% |
| 352 | 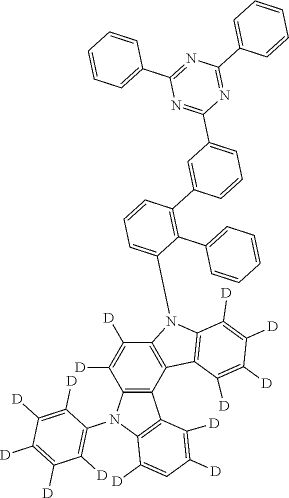 [SMILES: [2H]c1cc([2H])c2c(c1[2H])c1c3c4c([2H])c([2H])c([2H])c([2H])c4n(-c4cccc(-c5cccc(-c6nc(-c7ccccc7)nc(-c7ccccc7)n6)c5)c4-c4ccccc4)c3c([2H])c([2H])c1n2-c1c([2H])c([2H])c([2H])c([2H])c1[2H]] | 66% | 38% |
| 376 | 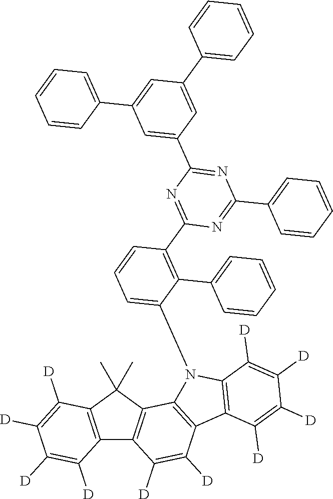 [SMILES: [2H]c1c([2H])c([2H])c2c(c1[2H])-c1c([2H])c([2H])c3c4c([2H])c([2H])c([2H])c([2H])c4n(-c4cccc(-c5nc(-c6ccccc6)nc(-c6cc(-c7ccccc7)cc(-c7ccccc7)c6)n5)c4-c4ccccc4)c3c1C2(C)C] | 71% | 24% |
| 461 | 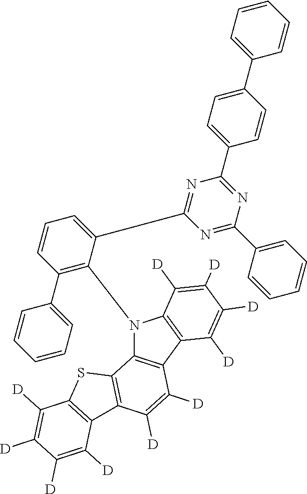 [SMILES: [2H]c1c([2H])c([2H])c2c(sc3c2c([2H])c([2H])c2c4c([2H])c([2H])c([2H])c([2H])c4n(-c4c(-c5ccccc5)cccc4-c4nc(-c5ccccc5)nc(-c5ccc(-c6ccccc6)cc5)n4)c32)c1[2H]] | 65% | 31% |
| 479 | 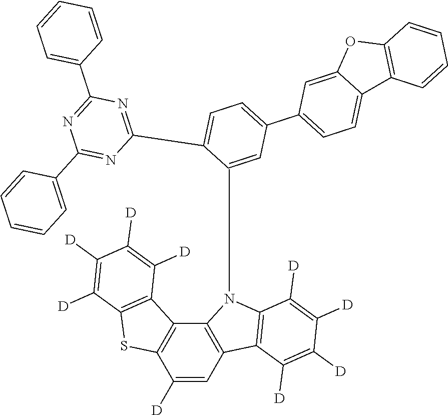 | 55% | 30% |
| 488 | 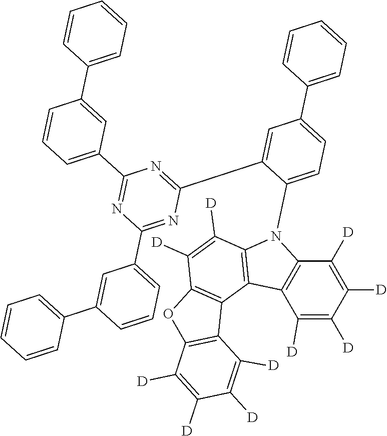 [SMILES: [2H]c1c([2H])c([2H])c2c(oc3c([2H])c([2H])c4c(c5c([2H])c([2H])c([2H])c([2H])c5n4-c4ccc(-c5ccccc5)cc4-c4nc(-c5cccc(-c6ccccc6)c5)nc(-c5cccc(-c6ccccc6)c5)n4)c32)c1[2H]] | 60% | 28% |
| 495 | 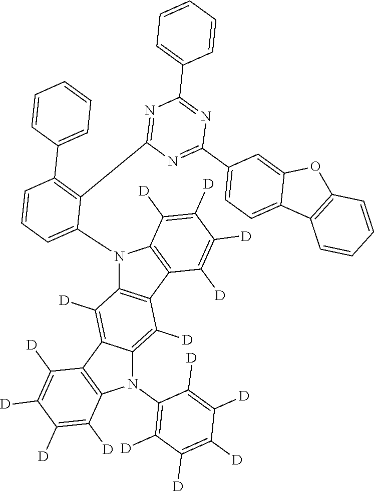 [SMILES: [2H]c1c([2H])c([2H])c(-n2c3c([2H])c([2H])c([2H])c([2H])c3c3c([2H])c4c(c([2H])c32)c2c([2H])c([2H])c([2H])c([2H])c2n4-c2cccc(-c3ccccc3)c2-c2nc(-c3ccccc3)nc(-c3ccc4c(c3)oc3ccccc34)n2)c([2H])c1[2H]] | 71% | 43% |
| 542 | 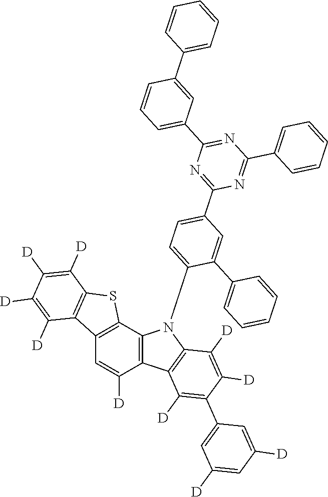 [SMILES: [2H]c1cc([2H])cc(-c2c([2H])c([2H])c3c(c2[2H])c2c([2H])cc4c(sc5c([2H])c([2H])c([2H])c([2H])c54)c2n3-c2ccc(-c3nc(-c4ccccc4)nc(-c4cccc(-c5ccccc5)c4)n3)cc2-c2ccccc2)c1] | 45% | 28% |
| 560 | 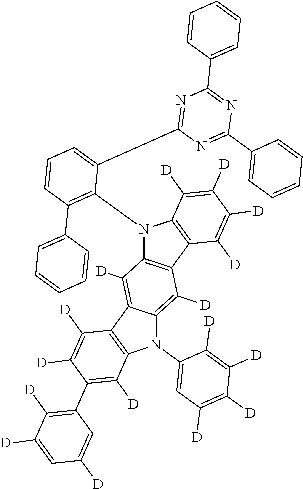 [SMILES: [2H]c1cc([2H])c([2H])c(-c2c([2H])c([2H])c3c4c([2H])c5c(c([2H])c4n(-c4cc([2H])c([2H])c([2H])c4[2H])c3c2[2H])c2c([2H])c([2H])c([2H])c([2H])c2n5-c2c(-c3ccccc3)cccc2-c2nc(-c3ccccc3)nc(-c3ccccc3)n2)c1] | 33% | 43% |

### Preparation Example 3

### Preparation of Compound 381C

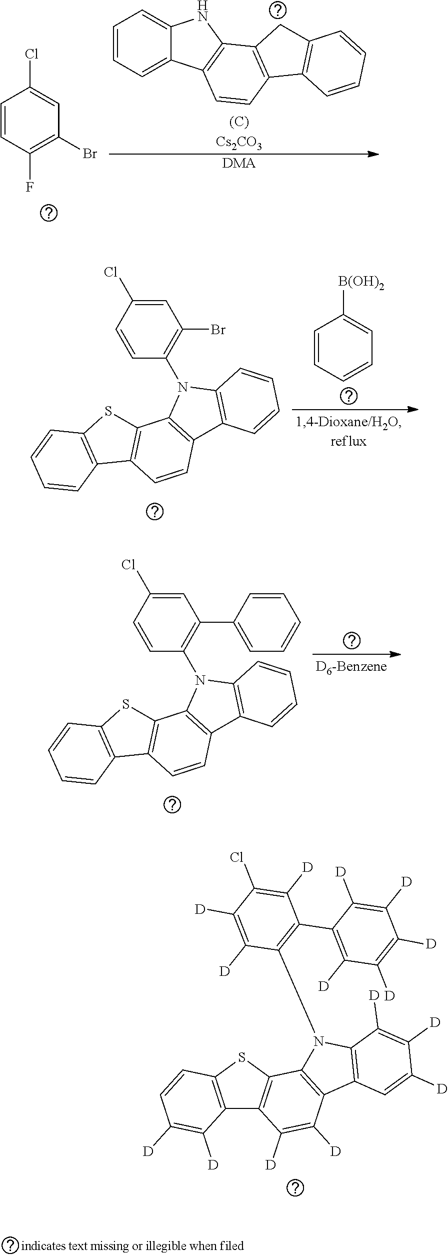

### 1) Preparation of Compound S-3

After Compound S-4 (2-bromo-4-chloro-1-fluorobenzene) (20 g, 95.49 mmol), 12H-benzo[4,5]thieno[2,3-a]carbazole (C) (25 g, 95.49 mmol), and Cs₂CO₃ (62 g, 190.98 mmol) were dissolved in 300 mL of DMA, the resulting solution was stirred at a reaction temperature of 160° C. for 15 hours. After the reaction was completed, the solution was cooled to room temperature, and an excess amount of H₂O was added thereto to precipitate a solid. The precipitated solid was filtered, washed with H₂O and methanol (MeOH), and then dried. The dried solid was dissolved in an excess amount of MC and then filtered through silica gel. The solvent was removed from the filtrate using a rotary evaporator and the residue was recrystallized with acetone to obtain 33 g of Compound S-3 as a pale yellow solid with a yield of 78%.

### 2) Preparation of Compound S-2

After Compound S-3 (33 g, 71.31 mmol), phenylboronic acid (D) (8.7 g, 71.31 mmol), Pd(PPh₃)₄ (4.1 g, 3.55 mmol), and K₂CO₃ (19 g, 142.62 mmol) were dissolved in 500 mL of 1,4-dioxane/100 mL of H₂O, the resulting solution was stirred at a reaction temperature of 100° C. for 6 hours. After the reaction was completed, the solution was cooled to room temperature, and the solvent was removed using a rotary evaporator. After the concentrated solution was dissolved in an excess amount of MC, extraction was performed with H₂O, and the organic layer was dried over anhydrous MgSO₄ and then filtered through silica gel. The solvent was removed from the filtrate using a rotary evaporator and the residue was recrystallized with EA to obtain 26 g of Compound S-2 as an ivory solid with a yield of 81%.

### 3) Preparation of Compound 381C

After Compound S-2 (26 g, 56.52 mmol) was dissolved in 130 mL of D₆-benzene, triflic acid (35 mL, 395.64 mmol) was slowly added thereto. After the reaction temperature was increased to 60° C., the solution was stirred for 1 hour. After the reaction was completed, the temperature of the reaction solution was lowered to 0° C., and the resulting product was neutralized by adding 30 mL of D₂O in which triethylamine (TEA) (31 mL, 226.08 mmol) had been dissolved thereto. After the organic layer was separated, it was dried over MgSO₄ and filtered through silica gel. The solvent was removed from the filtrate using a rotary evaporator, the residue was recrystallized with acetone to obtain 23 g of Compound 381C as an ivory solid with a yield of 85% (83% D substitution rate).

Target compounds were synthesized by performing the preparation in the same manner as in Preparation Example 3, except that Intermediate 6 in the following Table 4 was used instead of Compound S-4, Intermediate 7 in the following Table 4 was used instead of Compound (C), and Intermediate 8 in the following Table 4 was used instead of Compound (D).

TABLE 4

| Compound | Intermediate | Intermediate | Intermediate |
|---|---|---|---|
| No. | 6 | 7 | 8 |
| 396 | 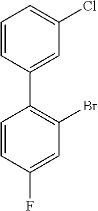 [SMILES: Fc1ccc(-c2cccc(Cl)c2)c(Br)c1] | 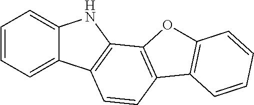 [SMILES: c1ccc2c(c1)[nH]c1c2ccc2c3ccccc3oc21] | 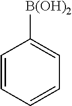 |
| 418 | 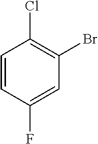 | 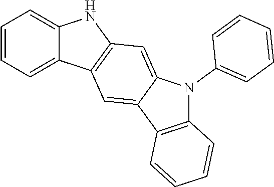 [SMILES: c1ccc(-n2c3ccccc3c3cc4c(cc32)[nH]c2ccccc24)cc1] | 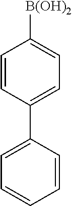 [SMILES: OB(O)c1ccc(-c2ccccc2)cc1] |
| 426 | 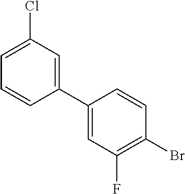 [SMILES: Fc1cc(-c2cccc(Cl)c2)ccc1Br] | 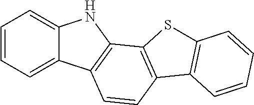 [SMILES: c1ccc2c(c1)[nH]c1c2ccc2c3ccccc3sc21] | 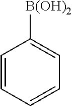 |
| 440 | 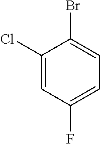 | 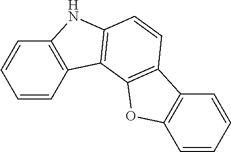 [SMILES: c1ccc2c(c1)[nH]c1ccc3c4ccccc4oc3c12] | 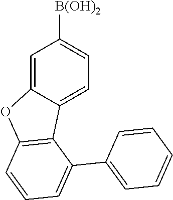 [SMILES: OB(O)c1ccc2c(c1)oc1cccc(-c3ccccc3)c12] |
| 448 | 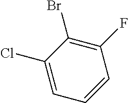 | 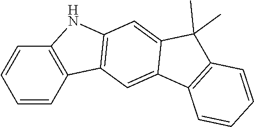 [SMILES: CC1(C)c2ccccc2-c2cc3c(cc21)[nH]c1ccccc13] | 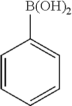 |
| 456 | 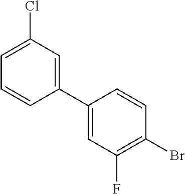 [SMILES: Fc1cc(-c2cccc(Cl)c2)ccc1Br] | 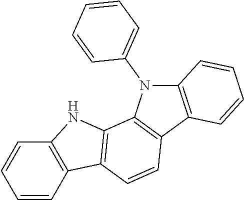 [SMILES: c1ccc(-n2c3ccccc3c3ccc4c5ccccc5[nH]c4c32)cc1] | 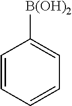 |
| 497 | 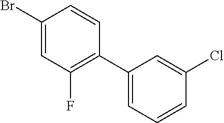 [SMILES: Fc1cc(Br)ccc1-c1cccc(Cl)c1] | 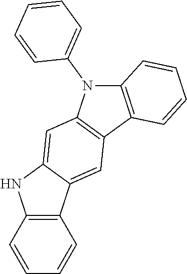 [SMILES: c1ccc(-n2c3ccccc3c3cc4c(cc32)[nH]c2ccccc24)cc1] | 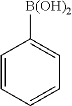 |
| 520 | 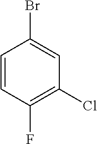 | 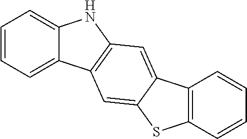 [SMILES: c1ccc2c(c1)[nH]c1cc3c(cc12)sc1ccccc13] | 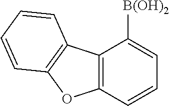 [SMILES: OB(O)c1cccc2oc3ccccc3c12] |

| Compound | | | D |
|---|---|---|---|
| No. | Target compound | yield | substitution rate |
| 396 | 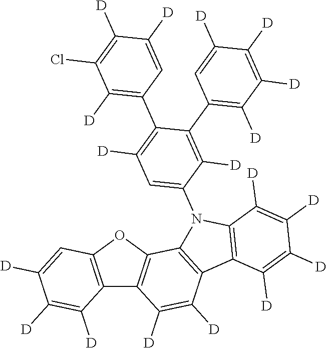 [SMILES: [2H]c1cc(-c2c([2H])c(-n3c4c([2H])c([2H])c([2H])c([2H])c4c4c([2H])c([2H])c5c(oc6cc([2H])c([2H])c([2H])c65)c43)cc([2H])c2-c2cc([2H])c([2H])c(Cl)c2[2H])c([2H])c([2H])c1[2H]] | 54% | 82% |
| 418 | 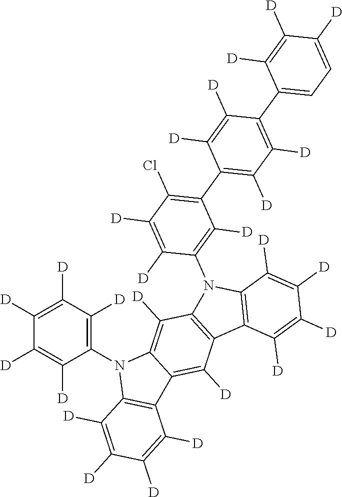 [SMILES: [2H]c1ccc(-c2c([2H])c([2H])c(-c3c([2H])c(-n4c5c([2H])c([2H])c([2H])c([2H])c5c5c([2H])c6c7c([2H])c([2H])c([2H])c([2H])c7n(-c7c([2H])c([2H])c([2H])c([2H])c7[2H])c6c([2H])c54)c([2H])c([2H])c3Cl)c([2H])c2[2H])c([2H])c1[2H]] | 66% | 93% |
| 426 | 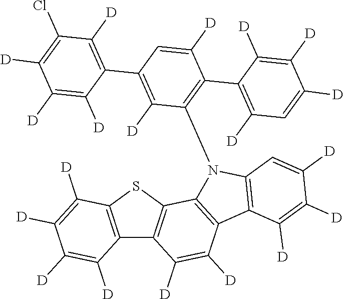 [SMILES: [2H]c1cc([2H])c(-c2c([2H])cc(-c3c([2H])c([2H])c([2H])c(Cl)c3[2H])c([2H])c2-n2c3cc([2H])c([2H])c([2H])c3c3c([2H])c([2H])c4c(sc5c([2H])c([2H])c([2H])c([2H])c54)c32)c([2H])c1[2H]] | 58% | 91% |
| 440 | 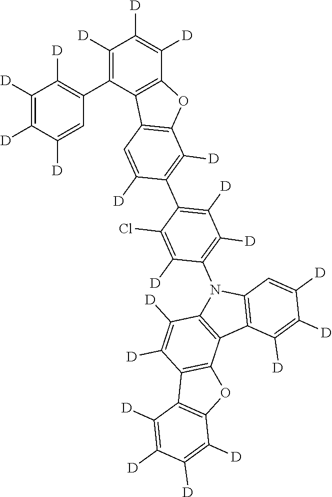 [SMILES: [2H]c1cc(-c2c([2H])c([2H])c([2H])c3oc4c([2H])c(-c5c([2H])c([2H])c(-n6c7cc([2H])c([2H])c([2H])c7c7c8oc9c([2H])c([2H])c([2H])c([2H])c9c8c([2H])c([2H])c76)c([2H])c5Cl)c([2H])cc4c23)c([2H])c([2H])c1[2H]] | 65% | 88% |
| 448 | 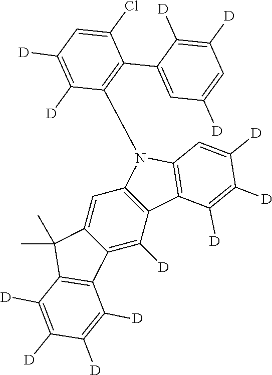 [SMILES: [2H]c1cc([2H])c([2H])c(-c2c(Cl)cc([2H])c([2H])c2-n2c3cc([2H])c([2H])c([2H])c3c3c([2H])c4c(cc32)C(C)(C)c2c([2H])c([2H])c([2H])c([2H])c2-4)c1] | 70% | 54% |
| 456 | 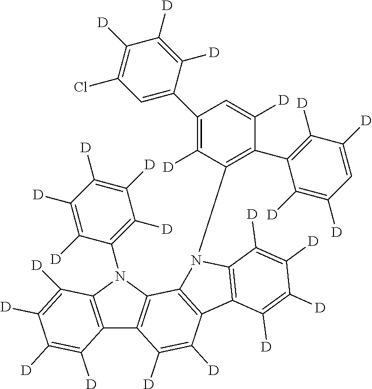 [SMILES: [2H]c1cc([2H])c([2H])c(-c2c([2H])cc(-c3cc(Cl)c([2H])c([2H])c3[2H])c([2H])c2-n2c3c([2H])c([2H])c([2H])c([2H])c3c3c([2H])c([2H])c4c5c([2H])c([2H])c([2H])c([2H])c5n(-c5c([2H])c([2H])c([2H])c([2H])c5[2H])c4c32)c1[2H]] | 59% | 89% |
| 497 | 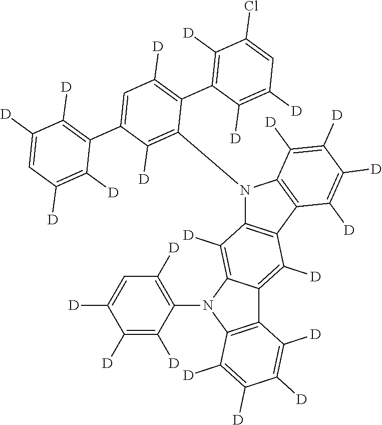 [SMILES: [2H]c1cc([2H])c(-n2c3c([2H])c([2H])c([2H])c([2H])c3c3c([2H])c4c5c([2H])c([2H])c([2H])c([2H])c5n(-c5c([2H])c(-c6c([2H])c([2H])cc([2H])c6[2H])cc([2H])c5-c5c([2H])c([2H])cc(Cl)c5[2H])c4c([2H])c32)c([2H])c1[2H]] | 60% | 85% |
| 520 | 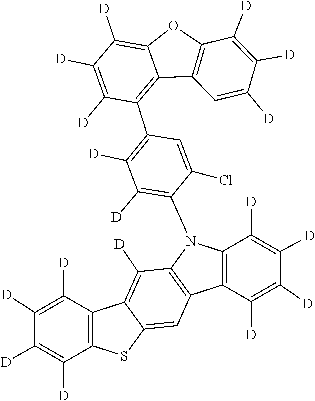 [SMILES: [2H]c1cc2c(oc3c([2H])c([2H])c([2H])c(-c4cc(Cl)c(-n5c6c([2H])c7c(cc6c6c([2H])c([2H])c([2H])c([2H])c65)sc5c([2H])c([2H])c([2H])c([2H])c57)c([2H])c4[2H])c32)c([2H])c1[2H]] | 62% | 85% |

### Preparation Example 4

### Preparation of Compound 381

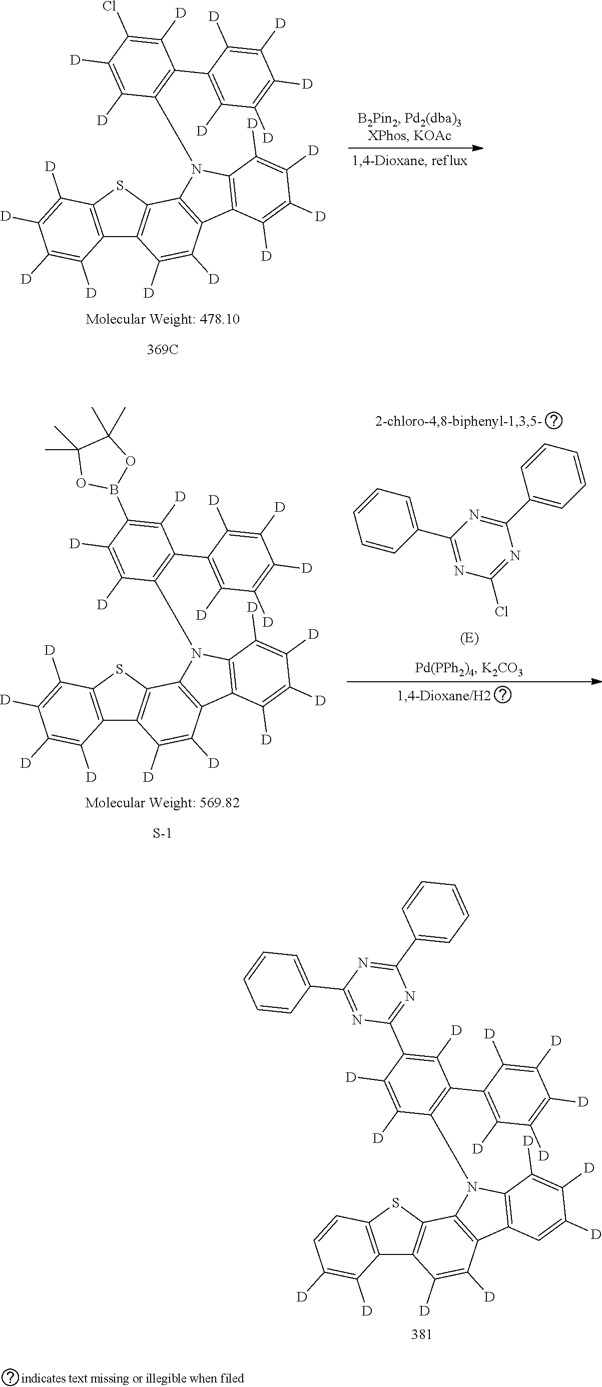

### 1) Preparation of Compound S-1

After Compound 381C (23 g, 48.12 mmol), bis(pinacolato)diboron (B₂Pin₂) (25 g, 96.24 mmol), Pd₂(dba)₃ (4.4 g, 4.82 mmol), XPhos (4.6 g, 9.62 mmol), KOAc (9.5 g, 96.24 mmol) were dissolved in 400 mL of 1,4-dioxane, the resulting solution was stirred at a reaction temperature of 100° C. for 15 hours. After the reaction was completed, the solution was cooled to room temperature, and the inorganic salts were filtered off. After the filtrate was concentrated using a rotary evaporator, the concentrate was dissolved in an excess amount of MC, and filtered through 10 silica gel. The solvent was removed from the filtrate using a rotary evaporator and the residue was recrystallized with MeOH to obtain 22 g of Compound S-1 as a yellow solid with a yield of 81%.

### 2) Preparation of Compound 381

After Compound S-1 (10 g, 17.57 mmol), 2-chloro-4,6-diphenyl-1,3,5-triazine (E) (6 g, 17.57 mmol), Pd(PPh₃)₄ (1 g, 0.88 mmol), and K₂CO₃ (4.8 g, 35.14 mmol) were dissolved in 120 mL of 1,4-dioxane/30 mL of H₂O, the resulting solution was stirred at 100° C. for 5 hours. After the reaction was completed, the solution was cooled to room temperature, and the solvent was removed using a rotary evaporator. After the concentrated solution was dissolved in an excess amount of MC, extraction was performed with H₂O, and the organic layer was dried over anhydrous MgSO₄ and then filtered through silica gel. The solvent was removed from the filtrate using a rotary evaporator and the residue was recrystallized with CB/acetone to obtain 8 g of Compound 381 as a white solid with a yield of 62%.

Final compounds were synthesized by performing the preparation in the same manner as in Preparation Example 4, except that Intermediate 9 in the following Table 5 was used instead of Compound 381C and Intermediate 10 in the following Table 5 was used instead of Compound (E).

TABLE 5

| Compound | Intermediate | Intermediate |
|---|---|---|
| No. | 9 | 10 |
| 396 | 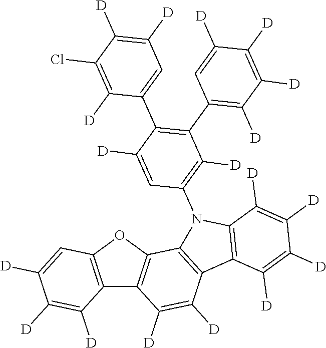 [SMILES: [2H]c1cc(-c2c([2H])c(-n3c4c([2H])c([2H])c([2H])c([2H])c4c4c([2H])c([2H])c5c(oc6cc([2H])c([2H])c([2H])c65)c43)cc([2H])c2-c2cc([2H])c([2H])c(Cl)c2[2H])c([2H])c([2H])c1[2H]] | 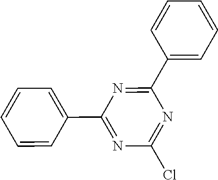 [SMILES: Clc1nc(-c2ccccc2)nc(-c2ccccc2)n1] |
| 418 | 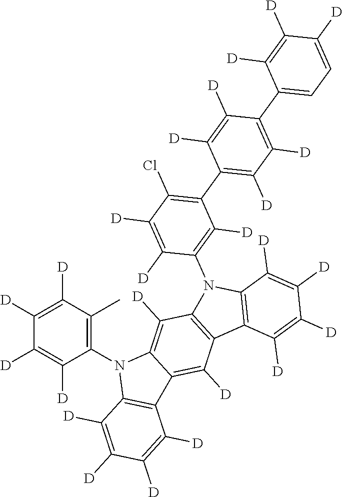 [SMILES: [2H]c1ccc(-c2c([2H])c([2H])c(-c3c([2H])c(-n4c5c([2H])c([2H])c([2H])c([2H])c5c5c([2H])c6c7c([2H])c([2H])c([2H])c([2H])c7n(-c7c([2H])c([2H])c([2H])c([2H])c7C)c6c([2H])c54)c([2H])c([2H])c3Cl)c([2H])c2[2H])c([2H])c1[2H]] | 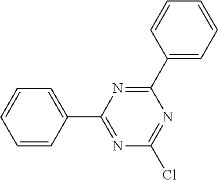 [SMILES: Clc1nc(-c2ccccc2)nc(-c2ccccc2)n1] |
| 426 | 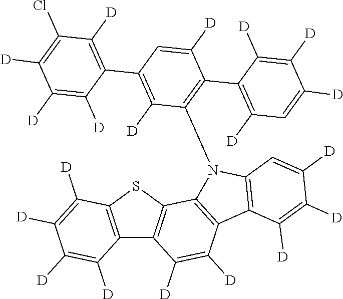 [SMILES: [2H]c1cc([2H])c(-c2c([2H])cc(-c3c([2H])c([2H])c([2H])c(Cl)c3[2H])c([2H])c2-n2c3cc([2H])c([2H])c([2H])c3c3c([2H])c([2H])c4c(sc5c([2H])c([2H])c([2H])c([2H])c54)c32)c([2H])c1[2H]] | 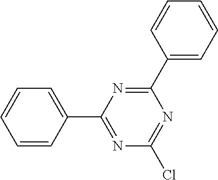 [SMILES: Clc1nc(-c2ccccc2)nc(-c2ccccc2)n1] |
| 440 | 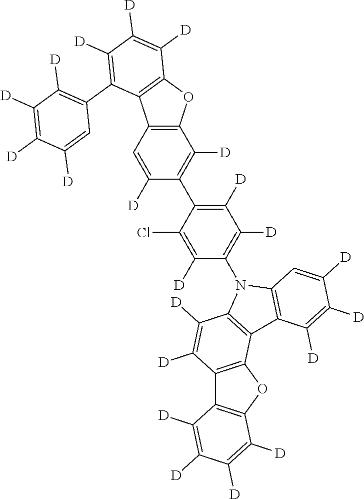 [SMILES: [2H]c1cc(-c2c([2H])c([2H])c([2H])c3oc4c([2H])c(-c5c([2H])c([2H])c(-n6c7cc([2H])c([2H])c([2H])c7c7c8oc9c([2H])c([2H])c([2H])c([2H])c9c8c([2H])c([2H])c76)c([2H])c5Cl)c([2H])cc4c23)c([2H])c([2H])c1[2H]] | 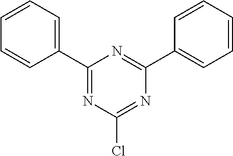 [SMILES: Clc1nc(-c2ccccc2)nc(-c2ccccc2)n1] |
| 448 | 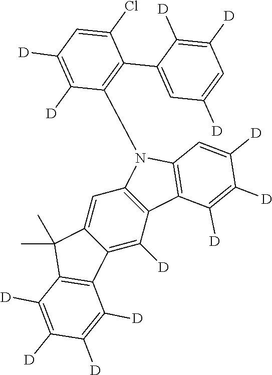 [SMILES: [2H]c1cc([2H])c([2H])c(-c2c(Cl)cc([2H])c([2H])c2-n2c3cc([2H])c([2H])c([2H])c3c3c([2H])c4c(cc32)C(C)(C)c2c([2H])c([2H])c([2H])c([2H])c2-4)c1] | 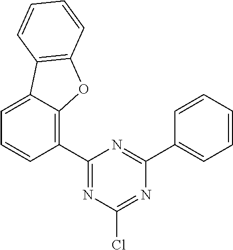 [SMILES: Clc1nc(-c2ccccc2)nc(-c2cccc3c2oc2ccccc23)n1] |
| 456 | 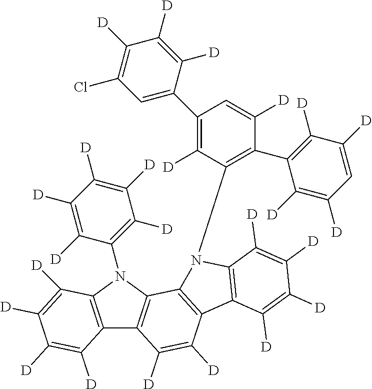 [SMILES: [2H]c1cc([2H])c([2H])c(-c2c([2H])cc(-c3cc(Cl)c([2H])c([2H])c3[2H])c([2H])c2-n2c3c([2H])c([2H])c([2H])c([2H])c3c3c([2H])c([2H])c4c5c([2H])c([2H])c([2H])c([2H])c5n(-c5c([2H])c([2H])c([2H])c([2H])c5[2H])c4c32)c1[2H]] | 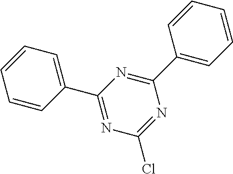 [SMILES: Clc1nc(-c2ccccc2)nc(-c2ccccc2)n1] |
| 497 | 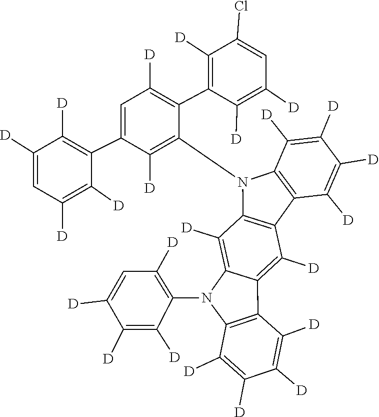 [SMILES: [2H]c1cc([2H])c(-n2c3c([2H])c([2H])c([2H])c([2H])c3c3c([2H])c4c5c([2H])c([2H])c([2H])c([2H])c5n(-c5c([2H])c(-c6c([2H])c([2H])cc([2H])c6[2H])cc([2H])c5-c5c([2H])c([2H])cc(Cl)c5[2H])c4c([2H])c32)c([2H])c1[2H]] | 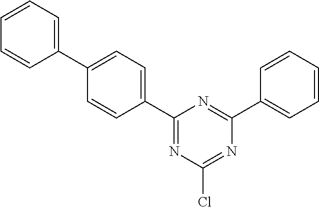 [SMILES: Clc1nc(-c2ccccc2)nc(-c2ccc(-c3ccccc3)cc2)n1] |
| 520 | 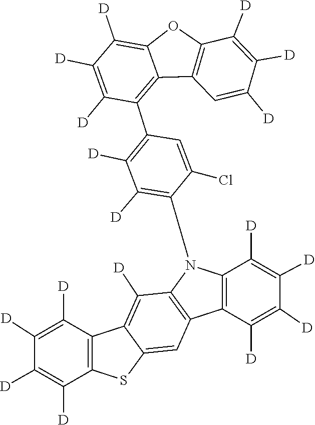 [SMILES: [2H]c1cc2c(oc3c([2H])c([2H])c([2H])c(-c4cc(Cl)c(-n5c6c([2H])c7c(cc6c6c([2H])c([2H])c([2H])c([2H])c65)sc5c([2H])c([2H])c([2H])c([2H])c57)c([2H])c4[2H])c32)c([2H])c1[2H]] | 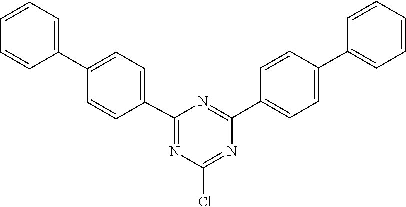 [SMILES: Clc1nc(-c2ccc(-c3ccccc3)cc2)nc(-c2ccc(-c3ccccc3)cc2)n1] |

| Compound | Final | | |
|---|---|---|---|
| No. | compound | Yield | D substitution rate |
| 396 | 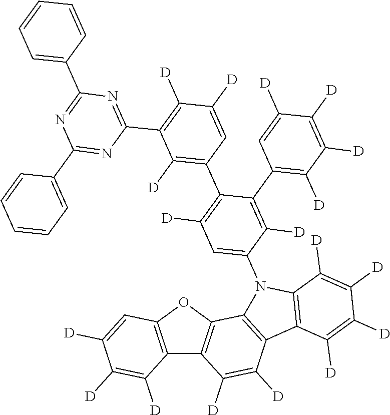 [SMILES: [2H]c1cc(-c2c([2H])c(-n3c4c([2H])c([2H])c([2H])c([2H])c4c4c([2H])c([2H])c5c(oc6cc([2H])c([2H])c([2H])c65)c43)cc([2H])c2-c2cc([2H])c([2H])c(-c3nc(-c4ccccc4)nc(-c4ccccc4)n3)c2[2H])c([2H])c([2H])c1[2H]] | 50% | 56% |
| 418 | 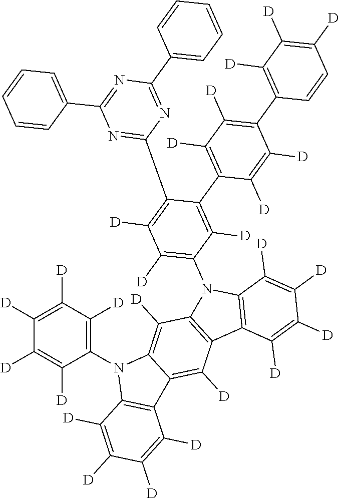 [SMILES: [2H]c1ccc(-c2c([2H])c([2H])c(-c3c([2H])c(-n4c5c([2H])c([2H])c([2H])c([2H])c5c5c([2H])c6c7c([2H])c([2H])c([2H])c([2H])c7n(-c7c([2H])c([2H])c([2H])c([2H])c7[2H])c6c([2H])c54)c([2H])c([2H])c3-c3nc(-c4ccccc4)nc(-c4ccccc4)n3)c([2H])c2[2H])c([2H])c1[2H]] | 62% | 68% |
| 426 | 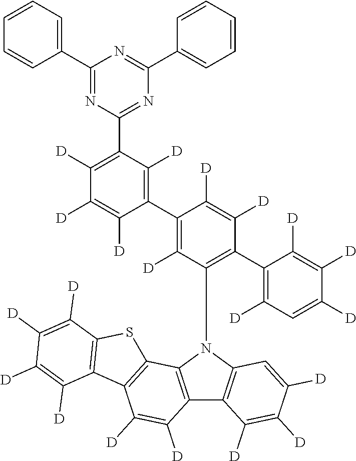 [SMILES: [2H]c1cc([2H])c(-c2c([2H])c([2H])c(-c3c([2H])c([2H])c([2H])c(-c4nc(-c5ccccc5)nc(-c5ccccc5)n4)c3[2H])c([2H])c2-n2c3cc([2H])c([2H])c([2H])c3c3c([2H])c([2H])c4c(sc5c([2H])c([2H])c([2H])c([2H])c54)c32)c([2H])c1[2H]] | 48% | 63% |
| 440 | 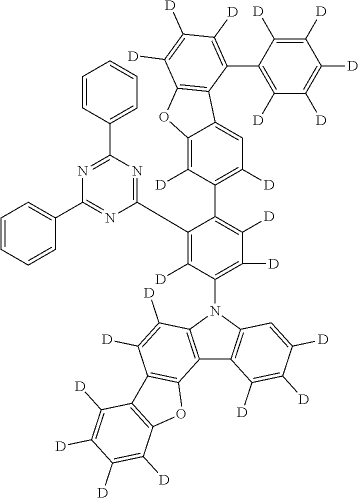 [SMILES: [2H]c1cc2c(c([2H])c1[2H])c1c3oc4c([2H])c([2H])c([2H])c([2H])c4c3c([2H])c([2H])c1n2-c1c([2H])c([2H])c(-c2c([2H])cc3c(oc4c([2H])c([2H])c([2H])c(-c5c([2H])c([2H])c([2H])c([2H])c5[2H])c43)c2[2H])c(-c2nc(-c3ccccc3)nc(-c3ccccc3)n2)c1[2H]] | 44% | 64% |
| 448 | 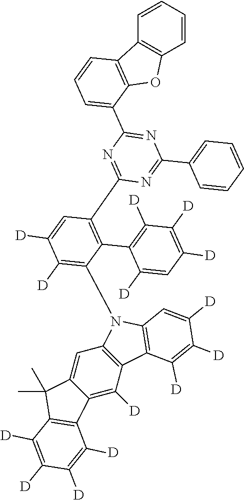 [SMILES: [2H]c1cc([2H])c(-c2c(-c3nc(-c4ccccc4)nc(-c4cccc5c4oc4ccccc45)n3)cc([2H])c([2H])c2-n2c3cc([2H])c([2H])c([2H])c3c3c([2H])c4c(cc32)C(C)(C)c2c([2H])c([2H])c([2H])c([2H])c2-4)c([2H])c1[2H]] | 59% | 39% |
| 456 | 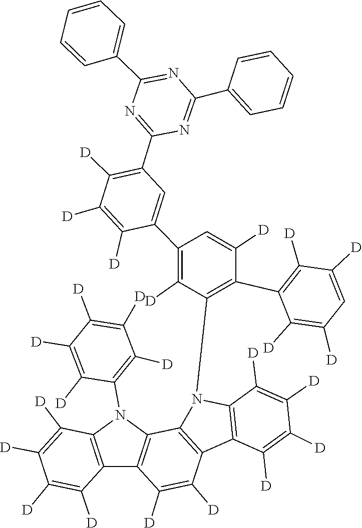 [SMILES: [2H]c1cc([2H])c([2H])c(-c2c([2H])cc(-c3cc(-c4nc(-c5ccccc5)nc(-c5ccccc5)n4)c([2H])c([2H])c3[2H])c([2H])c2-n2c3c([2H])c([2H])c([2H])c([2H])c3c3c([2H])c([2H])c4c5c([2H])c([2H])c([2H])c([2H])c5n(-c5c([2H])c([2H])c([2H])c([2H])c5[2H])c4c32)c1[2H]] | 63% | 65% |
| 497 | 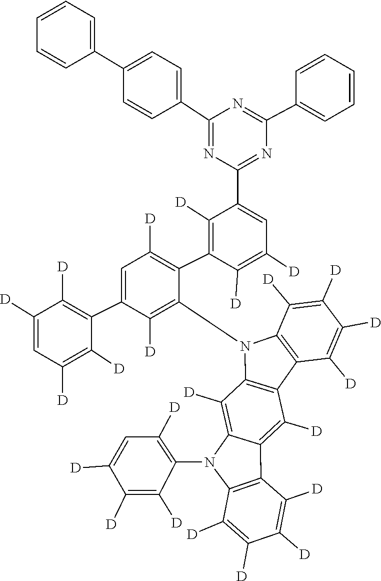 [SMILES: [2H]c1cc([2H])c(-n2c3c([2H])c([2H])c([2H])c([2H])c3c3c([2H])c4c5c([2H])c([2H])c([2H])c([2H])c5n(-c5c([2H])c(-c6c([2H])c([2H])cc([2H])c6[2H])cc([2H])c5-c5c([2H])c([2H])cc(-c6nc(-c7ccccc7)nc(-c7ccc(-c8ccccc8)cc7)n6)c5[2H])c4c([2H])c32)c([2H])c1[2H]] | 35% | 56% |
| 520 | 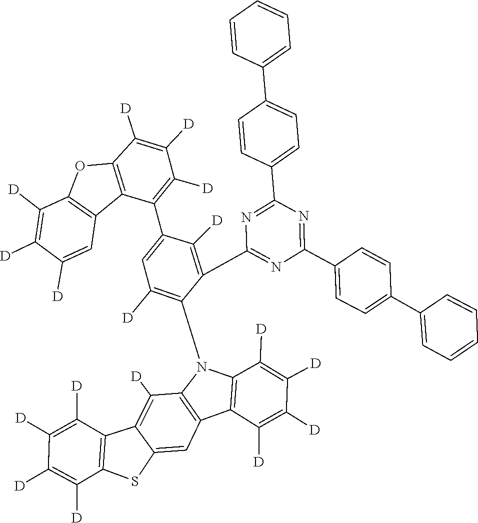 [SMILES: [2H]c1cc2c(oc3c([2H])c([2H])c([2H])c(-c4cc([2H])c(-n5c6c([2H])c7c(cc6c6c([2H])c([2H])c([2H])c([2H])c65)sc5c([2H])c([2H])c([2H])c([2H])c57)c(-c5nc(-c6ccc(-c7ccccc7)cc6)nc(-c6ccc(-c7ccccc7)cc6)n5)c4[2H])c32)c([2H])c1[2H]] | 49% | 45% |

### Preparation Example 5

### Preparation of Compound 2-2

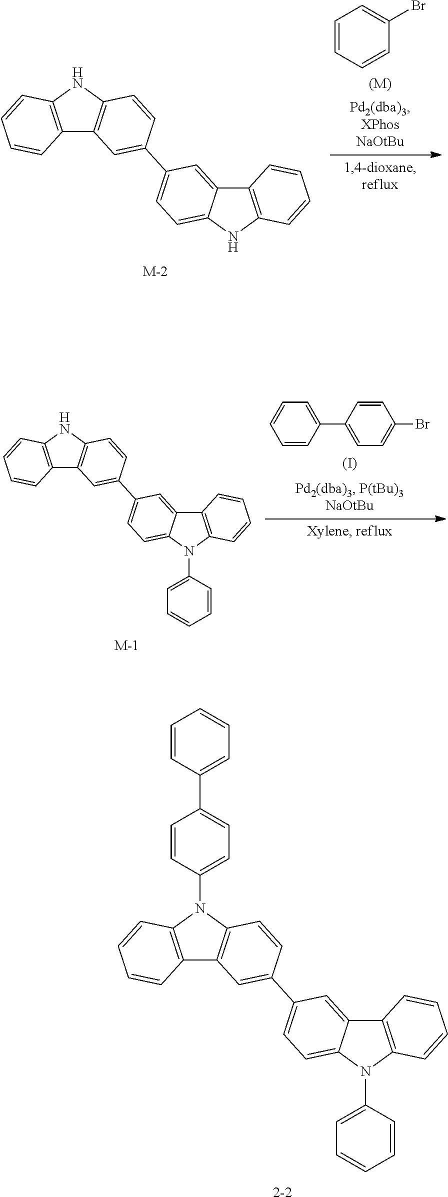

### 1) Preparation of Compound M-1

After Compound M-2 (9H,9′H-3,3′-bicarbazole) (20 g, 60.16 mmol), bromobenzene (H) (9.4 g, 60.16 mmol), Pd₂(dba)₃ (5.5 g, 6.02 mmol), XPhos (5.7 g, 12.03 mmol), and K₂CO₃ (12.5 g, 90.24 mmol) were dissolved in 300 mL of 1,4-dioxane, the resulting solution was stirred at a reaction temperature of 110° C. for 6 hours. After the reaction was completed, the solution was cooled to room temperature, and the solvent was removed using a rotary evaporator. After the concentrated solution was dissolved in an excess amount of MC, extraction was performed with H₂O, and the organic layer was dried over anhydrous MgSO₄ and then filtered through silica gel. The solvent was removed from the filtrate using a rotary evaporator and the residue was recrystallized with EA to obtain 20 g of Compound M-1 as a white solid with a yield of 81%.

### 2) Preparation of Compound 2-2

After Compound M-1 (20 g, 49.02 mmol), 4-bromo-1,1′-biphenyl (I) (12 g, 49.02 mmol), Pd₂(dba)₃ (4.5 g, 4.9 mmol), tri-tert-butylphosphine (P(tBu)₃) (1.9 g, 9.81 mmol), and sodium tert-butoxide (NaOtBu) (9.5 g, 98.04 mmol) were dissolved in 300 mL of toluene, the resulting solution was stirred at a reaction temperature of 110° C. for 15 hours. After the reaction was completed, the solution was cooled to room temperature, and the solvent was removed using a rotary evaporator. After the concentrated solution was dissolved in an excess amount of MC, extraction was performed with H₂O, and the organic layer was dried over anhydrous MgSO₄ and then filtered through silica gel. The solvent was removed from the filtrate using a rotary evaporator and the residue was recrystallized with CB to obtain 17 g of Compound 2-2 as a white solid with a yield of 63%.

Final compounds were synthesized by performing the preparation in the same manner as in Preparation Example 5, except that Intermediate 11 in the following Table 6 was used instead of Compound M-2, Intermediate 12 in the following Table 6 was used instead of Compound (H), and Intermediate 13 in the following Table 6 was used instead of Compound (I).

TABLE 6

| Compound | Intermediate | Intermediate | Intermediate |
|---|---|---|---|
| No. | 11 | 12 | 13 |
| 2-4 | 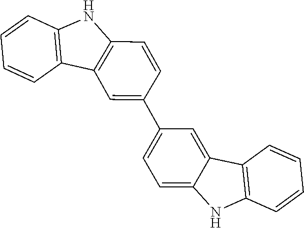 [SMILES: c1ccc2c(c1)[nH]c1ccc(-c3ccc4[nH]c5ccccc5c4c3)cc12] | 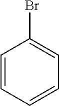 | 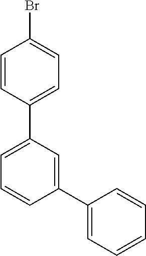 [SMILES: Brc1ccc(-c2cccc(-c3ccccc3)c2)cc1] |
| 2-15 | 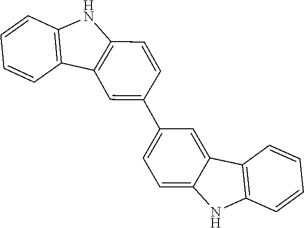 [SMILES: c1ccc2c(c1)[nH]c1ccc(-c3ccc4[nH]c5ccccc5c4c3)cc12] | 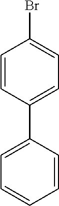 [SMILES: Brc1ccc(-c2ccccc2)cc1] | 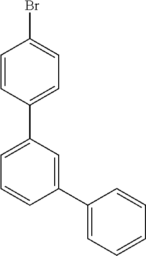 [SMILES: Brc1ccc(-c2cccc(-c3ccccc3)c2)cc1] |
| 2-31 | 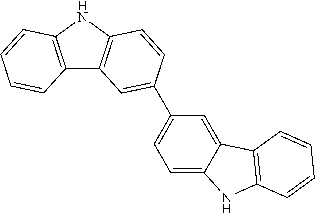 [SMILES: c1ccc2c(c1)[nH]c1ccc(-c3ccc4[nH]c5ccccc5c4c3)cc12] | 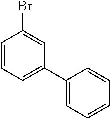 [SMILES: Brc1cccc(-c2ccccc2)c1] | 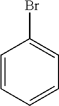 |
| 2-86 | 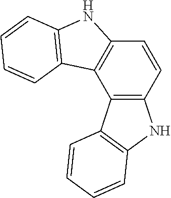 [SMILES: c1ccc2c(c1)[nH]c1ccc3[nH]c4ccccc4c3c12] | 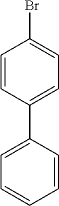 [SMILES: Brc1ccc(-c2ccccc2)cc1] | 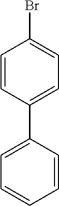 [SMILES: Brc1ccc(-c2ccccc2)cc1] |
| 2-107 | 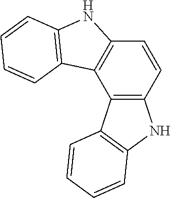 [SMILES: c1ccc2c(c1)[nH]c1ccc3[nH]c4ccccc4c3c12] | 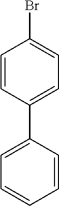 [SMILES: Brc1ccc(-c2ccccc2)cc1] | 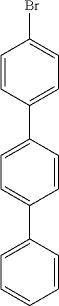 [SMILES: Brc1ccc(-c2ccc(-c3ccccc3)cc2)cc1] |
| 2-117 | 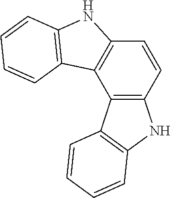 [SMILES: c1ccc2c(c1)[nH]c1ccc3[nH]c4ccccc4c3c12] | 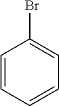 | 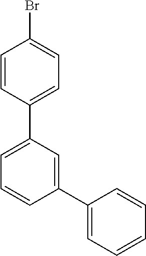 [SMILES: Brc1ccc(-c2cccc(-c3ccccc3)c2)cc1] |

| Compound No. | Final compound | Yield |
|---|---|---|
| 2-4 | 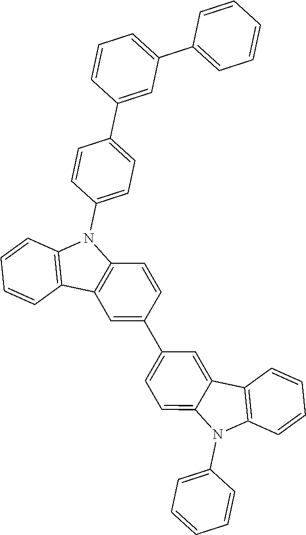 [SMILES: c1ccc(-c2cccc(-c3ccc(-n4c5ccccc5c5cc(-c6ccc7c(c6)c6ccccc6n7-c6ccccc6)ccc54)cc3)c2)cc1] | 51% |
| 2-15 | 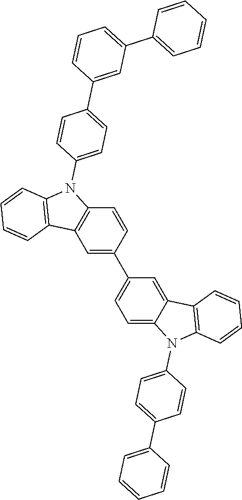 [SMILES: c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1] | 49% |
| 2-31 | 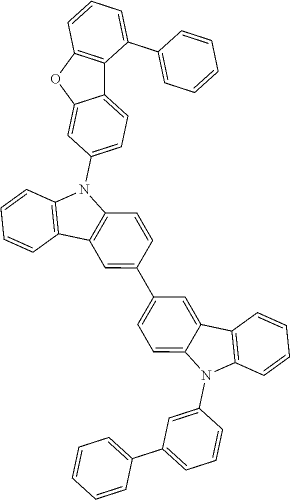 [SMILES: c1ccc(-c2cccc(-n3c4ccccc4c4cc(-c5ccc6c(c5)c5ccccc5n6-c5ccc6c(c5)oc5cccc(-c7ccccc7)c56)ccc43)c2)cc1] | 60% |
| 2-86 | 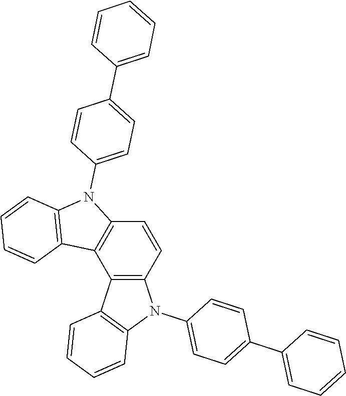 [SMILES: c1ccc(-c2ccc(-n3c4ccccc4c4c5c6ccccc6n(-c6ccc(-c7ccccc7)cc6)c5ccc43)cc2)cc1] | 55% |
| 2-107 | 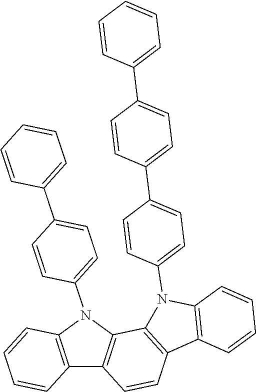 [SMILES: c1ccc(-c2ccc(-c3ccc(-n4c5ccccc5c5ccc6c7ccccc7n(-c7ccc(-c8ccccc8)cc7)c6c54)cc3)cc2)cc1] | 28% |
| 2-117 | 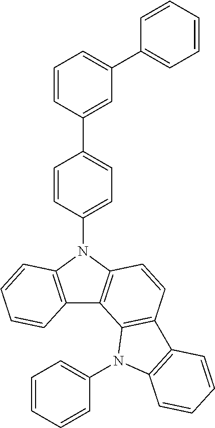 [SMILES: c1ccc(-c2cccc(-c3ccc(-n4c5ccccc5c5c4ccc4c6ccccc6n(-c6ccccc6)c45)cc3)c2)cc1] | 30% |

### Preparation Example 6

### Preparation of Compound 2-42

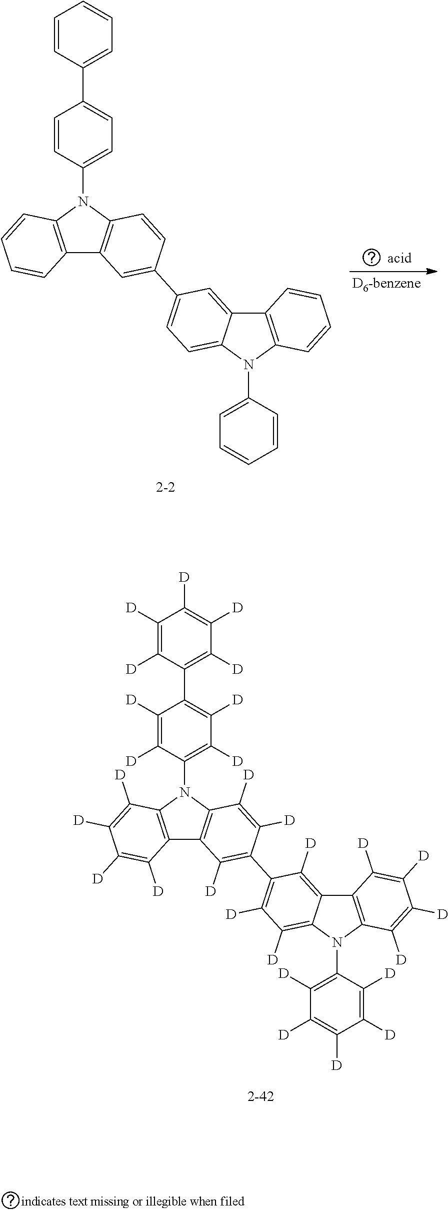 [SMILES: c1ccc(-c2ccc(-n3c4ccccc4c4cc(-c5ccc6c(c5)c5ccccc5n6-c5ccccc5)ccc43)cc2)cc1 ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c([2H])c2[2H])c([2H])c1[2H]]

After Compound 2-2 (9-([1,1′-biphenyl]-4-yl)-9′-phenyl-9H,9′H-3,3′-bicarbazole) (20 g, 35.66 mmol) was dissolved in 200 mL of D₆-benzene, triflic acid (23 mL, 249.69 mmol) was slowly added thereto. After the resulting mixture was stirred for 1 hour by increasing the reaction temperature to 60° C., the mixture was neutralized by adding a solution of TEA (20 mL, 142.64 mmol) dissolved in 20 mL of D₂O thereto. Extraction was performed by adding an excess amount of ethyl acetate (EA) thereto, and the organic layer was dried over anhydrous MgSO₄ and then filtered through silica gel. The solvent was removed from the filtrate using a rotary evaporator to obtain 19.8 g of Compound 2-42 as a brown solid with a yield of 94% (100% D substitution rate).

A final compound was synthesized by performing the preparation in the same manner as in Preparation Example 6, except that Intermediate 14 in the following Table 7 was used instead of Compound 2-2.

TABLE 7

| Compound | | | |
|---|---|---|---|
| No. | Intermediate 14 | | |
| 2-51 | 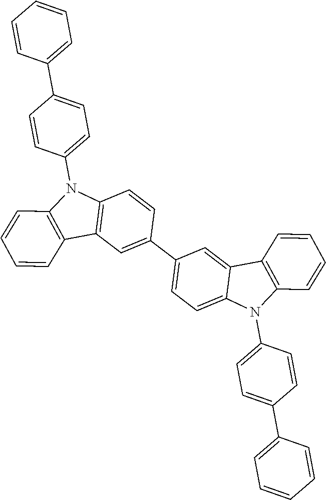 [SMILES: c1ccc(-c2ccc(-n3c4ccccc4c4cc(-c5ccc6c(c5)c5ccccc5n6-c5ccc(-c6ccccc6)cc5)ccc43)cc2)cc1] | | |
| 2-55 | 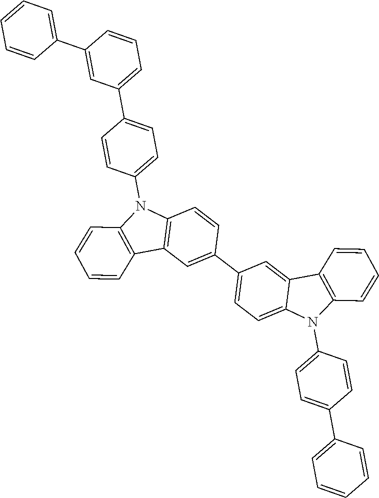 [SMILES: c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1] | | |
| 2-68 | 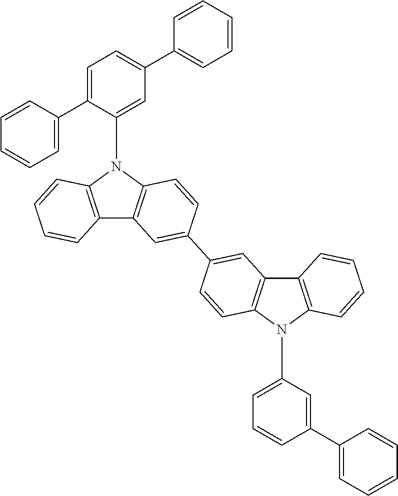 [SMILES: c1ccc(-c2cccc(-n3c4ccccc4c4cc(-c5ccc6c(c5)c5ccccc5n6-c5cc(-c6ccccc6)ccc5-c5ccccc5)ccc43)c2)cc1] | | |
| 2-123 | 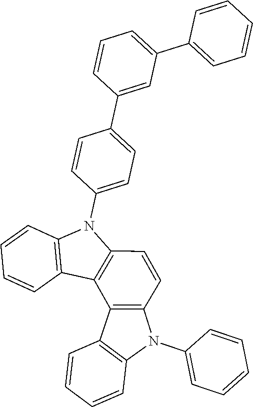 [SMILES: c1ccc(-c2cccc(-c3ccc(-n4c5ccccc5c5c6c7ccccc7n(-c7ccccc7)c6ccc54)cc3)c2)cc1] | | |
| 2-133 | 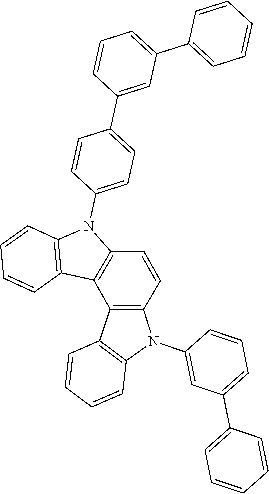 [SMILES: c1ccc(-c2cccc(-c3ccc(-n4c5ccccc5c5c6c7ccccc7n(-c7cccc(-c8ccccc8)c7)c6ccc54)cc3)c2)cc1] | | |
| 2-135 | 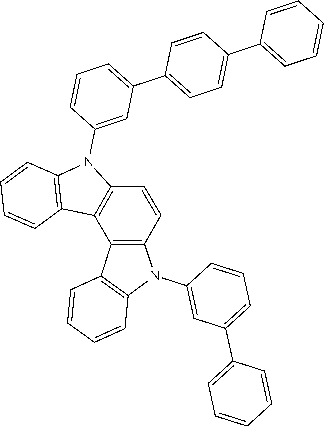 [SMILES: c1ccc(-c2ccc(-c3cccc(-n4c5ccccc5c5c6c7ccccc7n(-c7cccc(-c8ccccc8)c7)c6ccc54)c3)cc2)cc1] | | |
| 2-144 | 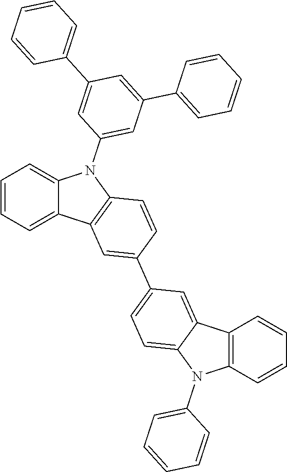 [SMILES: c1ccc(-c2cc(-c3ccccc3)cc(-n3c4ccccc4c4cc(-c5ccc6c(c5)c5ccccc5n6-c5ccccc5)ccc43)c2)cc1] | | |
| 2-151 | 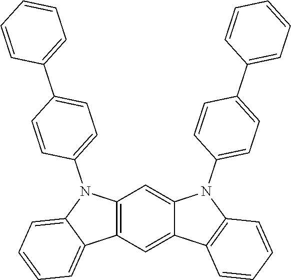 [SMILES: c1ccc(-c2ccc(-n3c4ccccc4c4cc5c6ccccc6n(-c6ccc(-c7ccccc7)cc6)c5cc43)cc2)cc1] | | |
| 2-155 | 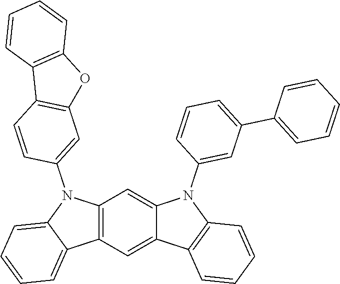 [SMILES: c1ccc(-c2cccc(-n3c4ccccc4c4cc5c6ccccc6n(-c6ccc7c(c6)oc6ccccc67)c5cc43)c2)cc1] | | |
| | | | D |
| Compound | | | substitution |
| No. | Final compound | Yield | rate |
| 2-51 | 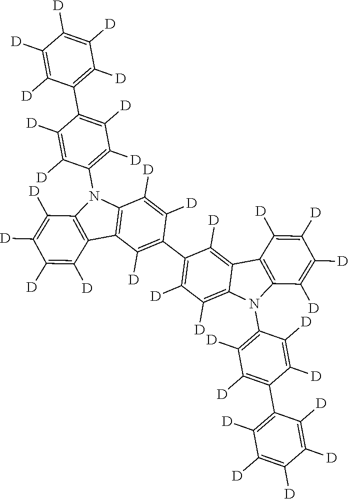 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c(-c6c([2H])c([2H])c([2H])c([2H])c6[2H])c([2H])c5[2H])c([2H])c([2H])c43)c([2H])c2[2H])c([2H])c1[2H]] | 77% | 100% |
| 2-55 | 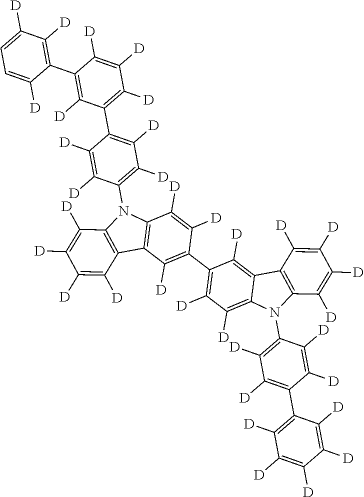 [SMILES: [2H]c1ccc([2H])c(-c2c([2H])c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c(-c7c([2H])c([2H])c([2H])c([2H])c7[2H])c([2H])c6[2H])c([2H])c([2H])c54)c([2H])c3[2H])c2[2H])c1[2H]] | 80% | 95% |
| 2-68 | 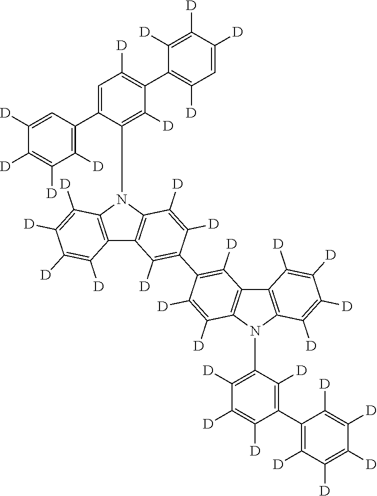 [SMILES: [2H]c1cc(-c2cc([2H])c(-c3c([2H])cc([2H])c([2H])c3[2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c(-c5c([2H])c([2H])c([2H])c([2H])c5[2H])c4[2H])c([2H])c([2H])c32)c([2H])c([2H])c1[2H]] | 68% | 92% |
| 2-123 | 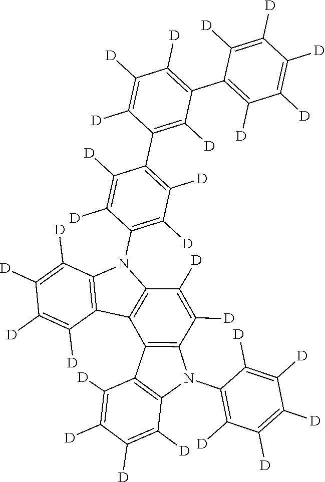 [SMILES: [2H]c1c([2H])c([2H])c(-c2c([2H])c([2H])c([2H])c(-c3c([2H])c([2H])c(-n4c5c([2H])c([2H])c([2H])c([2H])c5c5c6c7c([2H])c([2H])c([2H])c([2H])c7n(-c7c([2H])c([2H])c([2H])c([2H])c7[2H])c6c([2H])c([2H])c54)c([2H])c3[2H])c2[2H])c([2H])c1[2H]] | 75% | 100% |
| 2-133 | 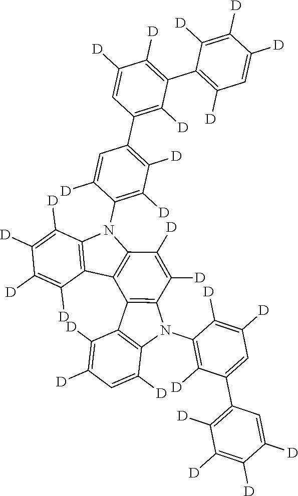 [SMILES: [2H]c1cc(-c2cc([2H])c([2H])c(-n3c4c([2H])cc([2H])c([2H])c4c4c5c6c([2H])c([2H])c([2H])c([2H])c6n(-c6c([2H])cc(-c7cc([2H])c([2H])c(-c8c([2H])cc([2H])c([2H])c8[2H])c7[2H])c([2H])c6[2H])c5c([2H])c([2H])c43)c2[2H])c([2H])c([2H])c1[2H]] | 66% | 81% |
| 2-135 | 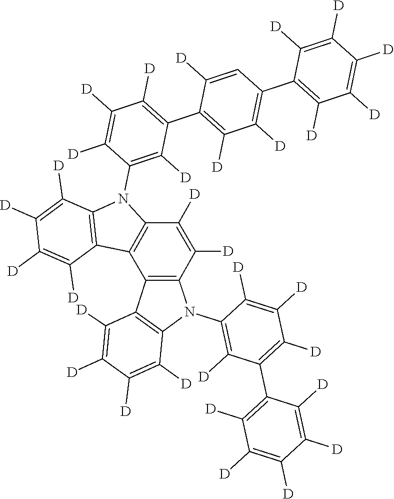 [SMILES: [2H]c1cc(-c2c([2H])c([2H])c([2H])c([2H])c2[2H])c([2H])c([2H])c1-c1c([2H])c([2H])c([2H])c(-n2c3c([2H])c([2H])c([2H])c([2H])c3c3c4c5c([2H])c([2H])c([2H])c([2H])c5n(-c5c([2H])c([2H])c([2H])c(-c6c([2H])c([2H])c([2H])c([2H])c6[2H])c5[2H])c4c([2H])c([2H])c32)c1[2H]] | 82% | 97% |
| 2-144 | 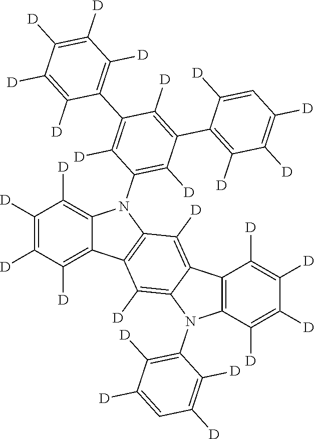 [SMILES: [2H]c1cc([2H])c(-c2c([2H])c(-c3c([2H])c([2H])c([2H])c([2H])c3[2H])c([2H])c(-n3c4c([2H])c([2H])c([2H])c([2H])c4c4c([2H])c5c(c([2H])c43)c3c([2H])c([2H])c([2H])c([2H])c3n5-c3c([2H])c([2H])cc([2H])c3[2H])c2[2H])c([2H])c1[2H]] | 66% | 89% |
| 2-151 | 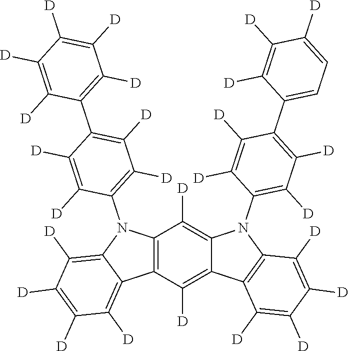 [SMILES: [2H]c1ccc(-c2c([2H])c([2H])c(-n3c4c([2H])c([2H])c([2H])c([2H])c4c4c([2H])c5c6c([2H])c([2H])c([2H])c([2H])c6n(-c6c([2H])c([2H])c(-c7c([2H])c([2H])c([2H])c([2H])c7[2H])c([2H])c6[2H])c5c([2H])c43)c([2H])c2[2H])c([2H])c1[2H]] | 70% | 93% |
| 2-155 | 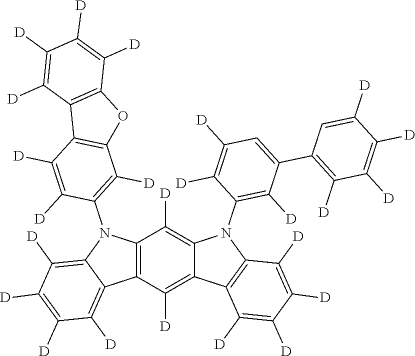 [SMILES: [2H]c1cc(-c2cc([2H])c([2H])c(-n3c4c([2H])c([2H])c([2H])c([2H])c4c4c([2H])c5c6c([2H])c([2H])c([2H])c([2H])c6n(-c6c([2H])c([2H])c7c(oc8c([2H])c([2H])c([2H])c([2H])c87)c6[2H])c5c([2H])c43)c2[2H])c([2H])c([2H])c1[2H]] | 72% | 88% |

The compounds synthesized in the Preparation Examples were confirmed through ¹H-NMR and FD-mass spectrometry. Tables 8 and 9 show the measured values of ¹H NMR (CDCl₃, 300 MHz), and Tables 10 and 11 show the measured values of field desorption mass spectrometry (FD-MS).

TABLE 8

| NO | ¹H NMR(CDCl₃, 300 MHz) |
|---|---|
| 9 | 7.21-7.23 (m, 4H), 7.31-7.39 (m, 3H), 7.41-7.45 (m, 3H), 7.57 (d, 1H), 7.69 (d, 1H), |
| | 7.82 (d, 1H), 7.98 (d, 1H), 8.01 (d, 1H), 8.08 (s, 1H), 8.36 (dd, 2H), 8.39-8.41 (m, 2H) |
| 12 | 7.19-7.20 (d, 4H), 7.41 (td, 1H), 7.49-7.51 (m, 6H), 7.61 (dd, 1H), 7.73 (t, 1H), 7.94 (s, |
| | 1H), 8.01 (d, 1H), 8.08 (s, 1H), 8.11 (dd, 2H), 8.38-8.40 (m, 4H) |
| 13 | 7.26-7.28 (m, 4H), 7.31 (d, 1H), 7.39 (td, 1H), 7.41-7.43 (m, 3H), 7.49-7.51 (m, 6H), |
| | 7.98 (d, 1H), 8.01 (dd, 2H), 8.08 (s, 1H), 8.11 (dd, 1H), 8.36-8.38 (m, 4H) |
| 17 | 7.20-7.22 (d, 4H), 7.30-7.35 (m, 3H), 7.40-7.43 (m, 3H), 7.50-7.51 (m, 2H), 7.52-7.53 |
| | (m, 2H), 7.98 (dd, 1H), 8.01 (s, 1H), 8.10 (d, 1H), 8.25 (d, 1H), 8.36 (dd, 1H), 8.98 |
| | (d, 1H) |
| 28 | 7.19-7.21 (d, 4H), 7.32-7.39 (m, 4H), 7.41-7.44 (m, 3H), 7.52 (dd, 2H), 7.70 (d, 1H), |
| | 7.80 (d, 1H), 7.89-7.90 (m, 2H), 8.18 (s, 1H), 8.26 (dd, 2H), 8.35-8.37 (m, 2H) |
| 29 | 1.69 (s, 6H), 7.19-7.20 (m, 4H), 7.28 (d, 1H), 7.38 (dd, 2H), 7.41-7.43 (m, 5H), 7.47 |
| | (d, 1H), 7.65 (d, 1H), 7.78 (dd, 1H), 7.90 (d, 1H), 8.01 (d, 1H), 8.08 (s, 1H), 8.11 (d, |
| | 1H), 8.36 (dd, 2H) |
| 32 | 7.20-7.22 (d, 4H), 7.29-7.30 (m, 3H), 7.47 (d, 1H), 7.50-7.52 (m, 2H), 7.66 (d, 1H), |
| | 7.77 (s, 1H), 7.80 (d, 1H), 7.89-7.90 (m, 2H), 8.18 (s, 1H), 8.26 (dd, 2H), 8.35-8.37 |
| | (m, 2H), 8.41 (d, 1H), 8.44 (d, 1H) |
| 38 | 7.16-7.17 (m, 3H), 7.35 (t, 1H), 7.46 (d, 1H), 7.50-7.52 (m, 6H), 7.50 (dd, 1H), 7.66 |
| | (d, 1H), 7.80 (t, 1H), 7.91 (d, 1H), 7.92 (d, 1H), 8.08-8.11 (m, 3H), 8.15 (s, 1H), 8.26 |
| | (dd, 2H), 8.35-8.37 (m, 4H), 8.55 (d, 1H) |
| 41 | 7.19-7.21 (d, 4H), 7.41 (t, 1H), 7.41-7.44 (m, 4H), 7.49 (s, 1H), 7.52 (dd, 2H), 8.01 (s, |
| | 1H), 8.08-8.10 (m, 2H), 8.35-8.37 (m, 4H) |
| 46 | 7.19-7.22 (d, 4H), 7.25 (dd, 2H), 7.47 (t, 1H), 7.50-7.52 (m, 4H), 7.55-7.58 (m, 2H), |
| | 7.62 (d, 1H), 7.78-7.80 (m, 2H), 7.89-7.90 (m, 2H), 8.11 (s, 1H), 8.16 (dd, 1H), 8.35- |
| | 8.37 (m, 2H), 8.41-8.42 (m, 2H) |
| 47 | 7.35-7.36 (m, 2H), 7.45-7.47 (m, 4H), 7.50-7.52 (m, 2H), 7.55-7.58 (m, 2H), 7.61 (dd, |
| | 1H), 7.75-7.76 (m, 3H), 7.80-7.82 (m, 2H), 7.94 (s, 1H), 8.10 (s, 1H), 8.14 (dd, 1H), |
| | 8.35-8.36 (m, 2H), 8.40-8.42 (m, 2H) |
| 59 | 7.41-7.43 (m, 4H), 7.49-7.50 (m, 5H), 7.60 (dd, 2H), 7.65 (s, 1H), 7.79 (dd, 2H), 7.96 |
| | (dd, 2H), 8.01 (s, 1H), 8.09 (d, 1H), 8.11 (d, 1H), 8.37-8.38 (m, 4H), 8.42 (dd, 2H) |
| 63 | 1.69 (s, 6H), 7.19-7.20 (m, 4H), 7.28 (d, 1H), 7.33-7.35 (m, 3H), 7.41 (t, 1H), 7.84 (s, |
| | 1H), 8.10 (d, 1H), 8.14 (dd, 1H), 8.19-8.22 (m, 3H), 8.35-8.36 (m, 2H), 8.40-8.42 (m, 2H) |
| 80 | 1.69 (s, 6H), 7.19-7.20 (m, 4H), 7.32-7.33 (m, 3H), 7.41 (td, 1H), 7.49-7.51 (m, 4H), |
| | 7.60 (dd, 2H), 7.96 (dd, 2H), 7.99 (dd, 2H), 8.01 (d, 1H), 8.08 (s, 1H), 8.11 (d, 1H), |
| | 8.36-8.37 (m, 4H) |
| 97 | 7.40-7.41 (m, 3H), 7.59-7.61 (m, 4H), 7.79 (dd, 2H), 8.02 (d, 1H), 8.10 (d, 1H), 8.18 |
| | (dd, 2H), 8.33 (dd, 2H), 8.39 (dd, 2H), 8.40 (s, 1H), 8.49 (dd, 2H), 9.00 (s, 2H) |
| 106 | 7.20-7.22 (m, 4H), 7.25-7.26 (m, 3H), 7.40-7.43 (m, 4H), 7.48-7.49 (m, 2H), 7.55 (d, |
| | 1H), 7.58 (dd, 2H), 7.61 (dd, 1H), 7.75-7.76 (m, 2H), 7.84 (s, 1H), 8.12 (s, 1H), 8.16 |
| | (dd, 1H), 8.37-8.38 (m, 2H), 8.41-8.42 (m, 2H) |
| 111 | 7.16-7.19 (m, 4H), 7.19-7.22 (m, 4H), 7.29-7.31 (m, 4H), 7.46 (d, 1H), 7.55 (d, 1H), |
| | 7.60 (dd, 2H), 7.68 (dd, 1H), 7.75-7.76 (m, 2H), 7.94 (dd, 2H), 8.10 (d, 1H), 8.19 (d, |
| | 1H), 8.40 (s, 1H), 8.55 (d, 1H) |
| 113 | 7.49-7.50 (m, 3H), 7.54-7.56 (m, 4H), 7.68 (td, 1H), 7.93 (d, 1H), 7.95 (dd, 2H), 8.01 |
| | (d, 1H), 8.03 (d, 1H), 8.10 (d, 1H), 8.22 (dd, 2H), 8.35-8.36 (m, 4H), 8.40 (dd, 1H) |
| 135 | 1.70 (s, 6H), 7.19-7.21 (m, 4H), 7.26-7.28 (m, 4H), 7.30 (dd, 2H), 7.34-7.35 (m, 3H), |
| | 7.43 (t, 1H), 7.46-7.48 (m, 2H), 7.55 (dd, 2H), 7.77 (s, 1H), 7.89 (s, 1H), 8.10 (s, 1H), |
| | 8.35-8.36 (m, 2H) |
| 136 | 7.40-7.42 (m, 3H), 7.50 (d, 1H), 7.52-7.55 (m, 4H), 7.60 (dd, 2H), 7.70 (d, 1H), 7.74 |
| | (d, 1H), 7.79 (dd, 2H), 7.94 (s, 1H), 8.03 (d, 1H), 8.10 (d, 1H), 8.22 (dd, 1H), 8.30 (m, |
| | 2H), 8.35 (m, 2H), 8.40 (s, 1H), 8.55 (d, 1H) |
| 138 | 7.35-7.38 (m, 4H), 7.41-7.46 (m, 4H), 7.55 (s, 1H), 7.60 (dd, 2H), 7.68-7.70 (m, 5H), |
| | 7.75-7.76 (m, 3H), 7.80 (d, 1H), 7.94 (dd, 2H), 8.03 (d, 1H), 8.10 (d, 1H), 8.16 (d, 1H), |
| | 8.36 (dd, 2H), 8.40 (s, 1H), 8.55 (dd, 2H) |
| 154 | 1.70 (s, 6H), 7.19-7.21 (m, 4H), 7.26-7.28 (m, 3H), 7.30 (d, 1H), 7.34-7.35 (m, 2H), |
| | 7.43 (t, 1H), 7.46-7.48 (m, 2H), 7.55 (dd, 2H), 7.70 (d, 1H), 8.10 (s, 1H), 8.14 (dd, |
| | 1H), 8.35-8.36 (m, 2H), 8.40-8.42 (m, 2H) |
| 166 | 7.25-7.29 (m, 3H), 7.32 (d, 1H), 7.34-7.35 (m, 2H), 7.41 (t, 1H), 7.47-7.48 (m, 2H), |
| | 7.57 (dd, 2H), 7.81 (d, 1H), 8.10 (s, 1H), 8.14 (dd, 1H), 8.35-8.36 (m, 2H), 8.40-8.42 |
| | (m, 2H) |
| 178 | 7.25 (dd, 4H), 7.41-4.42 (m, 4H), 7.49 (td, 1H), 7.49-7.50 (m, 6H), 7.60 (dd, 2H), 7.75 |
| | (dd, 2H), 7.80 (s, 1H), 7.90 (d, 1H), 7.96 (dd, 2H), 8.10 (d, 1H), 8.35-8.36 (m, 4H) |
| 179 | 7.16 (t, 1H), 7.35 (t, 1H), 7.40 (dd, 2H), 7.49-7.51 (m, 4H), 7.53 (dd, 1H), 7.55-7.58 |
| | (m, 4H), 7.93-7.94 (m, 2H), 8.06 (d, 1H), 8.09 (dd, 1H), 8.11 (d, 1H), 8.29 (d, 1H), |
| | 8.36-8.37 (m, 4H), 8.40 (s, 1H), 8.55 (d, 1H) |
| 180 | 7.20-7.23 (m, 4H), 7.25-7.27 (m, 3H), 7.30 (d, 1H), 7.34 (d, 1H), 7.37-7.39 (m, 3H), |
| | 7.47 (dd, 2H), 7.65-7.68 (m, 2H), 8.10 (s, 1H), 8.18 (dd, 1H), 8.35-8.36 (m, 2H), 8.40- |
| | 8.42 (m, 2H) |
| 186 | 7.16 (t, 1H), 7.35 (dd, 1H), 7.41-7.43 (m, 4H), 7.48-7.50 (m, 6H), 7.75 (dd, 2H), 7.77 |
| | (d, 1H), 7.87 (d, 1H), 7.89 (s, 1H), 7.99 (d, 1H), 8.06 (dd, 2H), 8.34 (s, 1H), 8.36-8.37 |
| | (m, 4H), 8.55 (dd, 1H) |
| 196 | 7.30-7.32 (m, 4H), 7.34-7.35 (m, 5H), 7.39-7.41 (m, 4H), 7.48 (dd, 2H), 7.57 (dd, 1H), |
| | 7.60-7.62 (m, 2H), 7.81 (d, 1H), 8.07 (s, 1H), 8.11 (dd, 1H), 8.29 (d, 1H), 8.35-8.36 (m, |
| | 2H), 8.40-8.42 (m, 2H), 8.55 (s, 1H) |
| 197 | 7.19-7.20 (m, 4H), 7.35 (t, 1H), 7.52-7.54 (m, 4H), 7.90 (d, 1H), 8.03 (dd, 2H), 8.10 |
| | (dd, 2H), 8.25 (d, 1H), 8.30 (dd, 2H), 8.40 (d, 1H), 8.43 (d, 1H), 8.97 (dd, 2H) |
| 207 | 7.39-7.40 (m, 4H), 7.42-7.45 (m, 5H), 7.62 (dd, 2H), 7.73-7.74 (m, 4H), 7.94 (s, 1H), |
| | 7.81 (d, 1H), 8.11 (dd, 1H), 8.29 (d, 1H), 8.35-8.36 (m, 2H), 8.39 (s, 1H), 8.40-8.42 |
| | (m, 2H), 8.55 (s, 1H) |
| 214 | 7.19-7.20 (m, 4H), 7.38 (t, 1H), 7.42-7.44 (, m, 3H), 7.51-7.53 (m, 3H), 7.92 (d, 1H), |
| | 8.10 (d, 1H), 8.39 (s, 1H), 8.40-8.41 (m, 4H) |
| 226 | 1.68 (s, 6H), 7.35-7.37 (m, 4H), 7.40-7.43 (m, 3H), 7.52 (d, 1H), 7.68 (d, 1H), 7.74 |
| | (dd, 2H), 7.81 (d, 1H), 7.85-7.88 (m, 2H), 7.97 (d, 1H), 8.11 (s, 1H), 8.29 (d, 1H), |
| | 8.35-8.36 (m, 2H), 8.40 (d, 1H) |
| 247 | 7.41-7.42 (m, 4H), 7.50-7.52 (m, 6H), 7.61 (dd, 2H), 7.63 (d, 1H), 7.72 (dd, 2H), 7.91 |
| | (s, 1H), 8.02 (d, 1H), 8.10 (d, 1H), 8.40 (s, 1H), 8.42-8.43 (m, 4H) |
| 251 | 7.15-7.17 (m, 4H), 7.20-7.23 (m, 3H), 7.35 (dd, 2H), 7.39-7.41 (m, 5H), 7.46 (d, 1H), |
| | 7.50-7.52 (m, 2H), 7.65 (d, 1H), 7.79 (dd, 2H), 8.02 (d, 1H), 8.10 (d, 1H), 8.19 (d, 1H), |
| | 8.33 (dd, 1H), 8.40 (s, 1H), 8.55 (d, 1H) |
| 253 | 7.17-7.19 (m, 4H), 7.22 (td, 1H), 7.35-7.41 (m, 5H), 7.47 (dd, 2H), 7.50 (dd, 2H), |
| | 7.65-7.67 (m, 3H), 8.01 (s, 1H), 8.34 (dd, 2H), 8.40 (dd, 2H) |
| 258 | 7.20-7.23 (m, 4H), 7.21-7.23 (m, 3H), 7.39-7.41 (m, 3H), 7.46 (d, 1H), 7.50-7.52 (m, |
| | 2H), 7.65 (d, 1H), 8.20 (d, 1H), 8.36 (dd, 2H), 8.39 (s, 1H), 8.40 (dd, 2H) |
| 278 | 7.16-7.19 (m, 4H), 7.22-7.25 (m, 4H), 7.35 (dd, 2H), 7.46 (d, 1H), 7.50-7.52 (m, 5H), |
| | 7.65 (d, 1H), 7.79 (dd, 2H), 8.02 (dd, 2H), 8.10 (s, 1H), 8.31-8.33 (m, 5H), 8.40 (s, |
| | 1H), 8.55 (d, 1H) |
| 298 | 1.68 (s, 6H), 1.72 (s, 6H), 7.34-7.37 (m, 6H), 7.40-7.42 (m, 3H), 7.52 (d, 1H), 7.68 (d, |
| | 1H), 7.74 (dd, 2H), 7.81 (d, 1H), 7.85-7.88 (m, 2H), 7.97 (d, 1H), 8.03 (s, 1H), 8.12 (d, |
| | 1H), 8.20 (d, 1H), 8.29 (d, 1H), 8.35-8.36 (m, 2H), 8.40 (dd, 2H), 8.51 (s, 1H) |
| 317 | 7.36-7.39 (m, 4H), 7.40-7.45 (m, 4H), 7.47 (dd, 2H), 7.49-7.52 (m, 4H), 7.66 (dd, 2H), |
| | 7.69 (d, 1H), 7.79 (dd, 2H), 8.02 (dd, 2H), 8.33 (d, 1H), 8.55 (d, 1H), 9.27 (s, 1H), 9.60 |
| | (d, 1H) |
| 335 | 7.30-7.33 (m, 4H), 7.41-7.42 (m, 2H), 7.47 (dd, 2H), 7.49-7.52 (m, 3H), 7.66 (dd, 2H), |
| | 7.69 (d, 1H), 7.79 (dd, 2H), 7.85 (dd, 1H), 7.94 (s, 1H), 8.02 (dd, 2H), 8.13 (d, 1H), |
| | 8.33 (d, 1H) |
| 352 | 7.29-7.31 (m, 4H), 7.35-7.39 (m, 3H), 7.45-7.47 (m, 4H), 7.55 (d, 1H), 7.66 (dd, 2H), |
| | 7.69 (d, 1H), 7.79 (dd, 2H), 7.85 (dd, 1H), 7.95 (s, 1H), 8.13 (d, 1H), 8.33 (dd, 2H), |
| | 8.38 (d, 1H) |
| 376 | 1.69 (s, 6H), 7.26-7.28 (m, 2H), 7.36-7.39 (m, 3H), 7.45-7.49 (m, 4H), 7.55-7.57 (m, |
| | 3H), 7.75 (dd, 2H), 7.77 (dd, 2H), 7.80 (d, 1H), 7.85 (dd, 1H), 7.92 (d, 1H), 8.01 (d, |
| | 1H), 8.04 (s, 3H), 8.22 (d, 1H), 8.36 (dd, 2H) |
| 396 | 7.27-7.29 (m, 2H), 7.35-7.39 (m, 3H), 7.58 (dd, 1H), 7.70-7.72 (m, 4H), 8.22 (d, 2H), |
| | 8.36 (dd, 2H) |
| 418 | 7.29 (d, 1H), 7.51-7.52 (m, 2H), 7.75 (dd, 2H), 7.91-7.92 (m, 4H), 8.23 (d, 1H), 8.37 |
| | (dd, 2H) |
| 426 | 7.29 (d, 1H), 7.51-7.52 (m, 3H), 7.75 (dd, 1H), 7.91-7.92 (m, 4H), 8.23 (d, 1H), 8.37 |
| | (dd, 2H) |
| 440 | 7.30 (dd, 1H), 7.70-7.75 (m, 4H), 7.88 (dd, 1H), 8.01 (s, 1H), 7.90-7.92 (m, 3H), 8.23 |
| | (d, 1H), 8.37 (dd, 2H) |
| 448 | 7.21-7.22 (t, 1H), 7.29-7.30 (m, 3H), 7.55-7.57 (m, 4H), 7.75 (dd, 2H), 7.77 (dd, 2H), |
| | 7.80-7.82 (m, 3H), 7.92 (d, 1H), 8.01-8.04 (m, 3H), 8.22 (d, 1H), 8.36 (dd, 2H), 8.55 |
| | (dd, 1H) |
| 456 | 7.28 (d, 1H), 7.36-7.37 (m, 3H), 7.70-7.75 (m, 4H), 7.88-7.90 (m, 3H), 8.23 (d, 1H), |
| | 8.37 (dd, 2H) |
| 461 | 7.21-7.22 (dd, 1H), 7.30-7.32 (m, 3H), 7.44 (dd, 1H), 7.55-7.57 (m, 4H), 7.75 (dd, 2H), |
| | 7.77 (dd, 2H), 7.80-7.82 (m, 3H), 8.01-8.04 (m, 3H), 8.36 (dd, 2H), 8.55 (dd, 1H) |
| 479 | 7.21-7.22 (dd, 1H), 7.29-7.31 (m, 2H), 7.55-7.57 (m, 4H), 7.75 (dd, 2H), 7.77 (dd, 2H), |
| | 7.80-7.82 (m, 3H), 8.01-8.04 (m, 3H), 8.21 (dd, 1H), 8.36 (dd, 2H), 8.55 (dd, 1H) |
| 488 | 7.30-7.31 (m, 2H), 7.55 (dd, 1H), 7.69-7.70 (m, 3H), 7.75 (dd, 2H), 7.77 (dd, 2H), 7.82 |
| | (s, 1H), 8.01-8.04 (m, 3H), 8.21 (dd, 1H), 8.36 (dd, 2H), 8.55 (dd, 1H) |
| 495 | 7.28-7.31 (m, 4H), 7.50 (s, 1H), 7.60-7.62 (m, 2H), 7.71 (dd, 1H), 8.01-8.04 (m, |
| | 3H), 8.10-8.11 (m, 4H), 8.21 (dd, 1H), 8.36 (dd, 2H), 8.41 (s, 1H), 8.55 (dd, 1H) |
| 497 | 7.28 (dd, 1H), 7.36-7.37 (m, 4H), 7.70-7.75 (m, 4H), 7.88-7.90 (m, 3H), 8.11-8.12 (m, |
| | 3H), 8.23 (d, 1H), 8.37 (dd, 2H) |
| 520 | 7.36-7.37 (m, 3H), 7.40-7.45 (m, 3H), 7.47 (dd, 2H), 7.49-7.52 (m, 4H), 7.66 (dd, 2H), |
| | 7.69 (d, 1H), 7.79 (dd, 2H), 8.02 (dd, 2H), 8.33 (d, 1H), 8.55 (d, 1H), |
| 542 | 7.22-7.24 (m, 4H), 7.32 (t, 1H), 7.40-7.44 (m, 3H), 7.50-7.52 (m, 4H), 7.70-7.75 (m, |
| | 2H), 7.75 (dd, 2H), 7.80-7.82 (m, 3H), 7.90 (s, 1H), 8.01-8.04 (m, 3H), 8.36 (dd, 2H), |
| | 8.55 (dd, 1H) |
| 560 | 7.23-7.26 (m, 3H), 7.40 (m, 2H), 7.42-7.45 (m, 5H), 7.55 (dd, 1H), 7.60-7.62 (m, 4H), |
| | 7.79 (d, 1H), 8.02 (dd, 1H), 8.34-8.35 (m, 4H) |

TABLE 9

| NO | ¹H NMR(CDCl₃, 300 MHz) |
|---|---|
| 2-2 | 7.20-7.24 (m, 4H), 7.30-7.33 (m, 5H), 7.41-7.42 (m, 3H), 7.75 (dd, 2H), 7.89 (s, 2H), |
| | 7.94-7.96 (m, 4H), 8.00-8.03 (m, 2H), 8.11 (d, 2H), 8.19 (d, 1H), 8.30 (d, 1H), 8.55 |
| | (dd, 2H) |
| 2-4 | 7.21-7.23 (m, 3H), 7.30-7.33 (m, 6H), 7.38 (s, 1H), 7.41-7.43 (m, 4H), 7.75 (dd, 2H), |
| | 7.87-7.90 (m, 4H), 7.94-7.96 (m, 4H), 8.00-8.03 (m, 2H), 8.11 (d, 2H), 8.19 (d, 1H), |
| | 8.30 (d, 1H), 8.55 (dd, 2H) |
| 2-15 | 7.19-7.21 (m, 5H), 7.25-7.27 (m, 3H), 7.30-7.33 (m, 6H), 7.38 (s, 1H), 7.41-7.43 (m, |
| | 4H), 7.75 (dd, 2H), 7.87-7.90 (m, 4H), 7.94-7.96 (m, 4H), 8.00-8.03 (m, 2H), 8.11 (d, |
| | 2H), 8.19 (d, 1H), 8.30 (d, 1H), 8.55 (dd, 2H) |
| 2-31 | 7.19-7.21 (m, 3H), 7.23-7.25 (m, 3H), 7.30-7.32 (m, 6H), 7.41-7.43 (m, 4H), 7.75 (dd, |
| | 2H), 7.87-7.90 (m, 4H), 7.94-7.96 (m, 4H), 8.00-8.03 (m, 2H), 8.11 (d, 2H), 8.19 (d, |
| | 1H), 8.25 (s, 1H), 8.30 (d, 1H), 8.55 (dd, 2H) |
| 2-51 | Deuterium content of 100% and there is no ¹H NMR peak |
| 2-55 | 7.49 (s, 1H), 8.52 (s, 1H), |
| 2-68 | 7.52 (s, 1H), 7.66 (m, 2H) |
| 2-86 | 7.16 (t, 2H), 7.23-7.26 (m, 3H), 7.35-7.37 (m, 4H), 7.40-7.41 (m, 5H), 7.49 (dd, 2H), |
| | 7.65 (dd, 2H), 7.75 (d, 2H), 7.91-7.95 (m, 4H), 7.99 (dd, 2H), 8.56 (dd, 2H) |
| 2-107 | 7.19 (t, 2H), 7.20-7.21 (m, 4H), 7.23-7.26 (m, 3H), 7.35-7.37 (m, 4H), 7.40-7.41 (m, |
| | 5H), 7.49 (dd, 2H), 7.65 (dd, 2H), 7.75 (d, 2H), 7.91-7.95 (m, 4H), 7.99 (dd, 2H), 8.56 |
| | (dd, 2H) |
| 2-117 | 7.16 (t, 2H), 7.23-7.26 (m, 3H), 7.35-7.37 (m, 4H), 7.40-7.41 (m, 5H), 7.49 (dd, 2H), |
| | 7.65 (dd, 2H), 7.75 (d, 2H), 7.81 (s, 1H), 7.91-7.95 (m, 3H), 7.99 (dd, 2H), 8.56 (dd, 2H) |
| 2-123 | Deuterium content of 100% and there is no ¹H NMR peak |
| 2-133 | 7.21-7.23 (m, 3H), 7.38 (s, 1H), 7.55 (m, 2H) |
| 2-135 | 7.50 (s, 1H) |
| 2-144 | 7.16 (s, 1H), 7.29 (m, 2H) |
| 2-151 | 7.55 (s, 1H) |
| 2-155 | 7.26 (s, 1H), 7.52 (m, 2H) |

TABLE 10

| Compound | FD-MS |
|---|---|
| 1 | m/z = 667.87(C₄₅H₁₉D₁₀N₄S) |
| 2 | m/z = 666.87(C₄₅H₁₈D₁₀N₄S) |
| 3 | m/z = 668.87(C₄₅H₂₀D₁₀N₄S) |
| 4 | m/z = 666.87(C₄₅H₁₈D₁₀N₄S) |
| 5 | m/z = 666.87(C₄₅H₁₈D₁₀N₄S) |
| 6 | m/z = 667.87(C₄₅H₁₉D₁₀N₄S) |
| 7 | m/z = 742.97(C₅₁H₂₂D₁₀N₄S) |
| 8 | m/z = 818.05(C₅₇H₂₅D₁₀N₄S) |
| 9 | m/z = 756.95(C₅₁H₂₀D₁₀N₄SO) |
| 10 | m/z = 820.06(C₅₇H₂₇D₁₀N₄S) |
| 11 | m/z = 758.97(C₅₁H₂₄D₁₀N₅S) |
| 12 | m/z = 743.97(C₅₁H₂₃D₁₀N₄S) |
| 13 | m/z = 833.05(C₅₇H₂₄D₁₀N₄SO) |
| 14 | m/z = 742.97(C₅₁H₂₂D₁₀N₄S) |
| 15 | m/z = 820.06(C₅₇H₂₇D₁₀N₄S) |
| 16 | m/z = 775.01(C₅₁H₂₂D₁₀N₄S₂) |
| 17 | m/z = 716.93(C₄₉H₂₀D₁₀N₄S) |
| 18 | m/z = 850.11(C₅₇H₂₅D₁₀N₄S₂) |
| 19 | m/z = 718.93(C₄₉H₂₂D₁₀N₄S) |
| 20 | m/z = 743.97(C₅₁H₂₃D₁₀N₄S) |
| 21 | m/z = 650.81(C₄₅H₁₈D₁₀N₄O) |
| 22 | m/z = 650.81(C₄₅H₁₈D₁₀N₄O) |
| 23 | m/z = 651.81(C₄₅H₁₉D₁₀N₄O) |
| 24 | m/z = 652.81(C₄₅H₂₀D₁₀N₄O) |
| 25 | m/z = 651.81(C₄₅H₁₉D₁₀N₄O) |
| 26 | m/z = 650.81(C₄₅H₁₈D₁₀N₄O) |
| 27 | m/z = 727.91(C₅₁H₂₃D₁₀N₄O) |
| 28 | m/z = 726.91(C₅₁H₂₂D₁₀N₄O) |
| 29 | m/z = 767.97(C₅₄H₂₇D₁₀N₄O) |
| 30 | m/z = 726.91(C₅₁H₂₂D₁₀N₄O) |
| 31 | m/z = 740.90(C₅₁H₂₂D₁₀N₅O) |
| 32 | m/z = 726.91(C₅₁H₂₂D₁₀N₄O) |
| 33 | m/z = 741.89(C₅₁H₂₁D₁₀N₄O₂) |
| 34 | m/z = 729.91(C₅₁H₂₅D₁₀N₄O) |
| 35 | m/z = 805.00(C₅₇H₂₈D₁₀N₄O) |
| 36 | m/z = 834.05(C₅₇H₂₅D₁₀N₄OS) |
| 37 | m/z = 700.87(C₄₉H₂₀D₁₀N₄O) |
| 38 | m/z = 818.00(C₅₇H₂₇D₁₀N₅O) |
| 39 | m/z = 700.87(C₄₉H₂₀D₁₀N₄O) |
| 40 | m/z = 728.91(C₅₁H₂₄D₁₀N₄O) |
| 41 | m/z = 731.95(C₅₁H₁₉D₁₅N₅) |
| 42 | m/z = 734.95(C₅₁H₂₂D₁₅N₅) |
| 43 | m/z = 732.95(C₅₁H₂₀D₁₅N₅) |
| 44 | m/z = 731.95(C₅₁H₁₉D₁₅N₅) |
| 45 | m/z = 734.95(C₅₁H₂₂D₁₅N₅) |
| 46 | m/z = 732.95(C₅₁H₂₀D₁₅N₅) |
| 47 | m/z = 810.05(C₅₇H₂₅D₁₅N₅) |
| 48 | m/z = 808.05(C₅₇H₂₃D₁₅N₅) |
| 49 | m/z = 821.03(C₅₇H₂₀D₁₅N₅O) |
| 50 | m/z = 808.05(C₅₇H₂₃D₁₅N₅) |
| 51 | m/z = 820.05(C₅₇H₂₁D₁₅N₆) |
| 52 | m/z = 812.05(C₅₇H₂₇D₁₅N₅) |
| 53 | m/z = 901.13(C₆₃H₂₈D₁₅N₅O) |
| 54 | m/z = 808.05(C₅₇H₂₃D₁₅N₅) |
| 55 | m/z = 885.15(C₆₃H₂₈D₁₅N₅) |
| 56 | m/z = 900.13(C₆₃H₂₇D₁₅N₅O) |
| 57 | m/z = 782.01(C₅₅H₂₁D₁₅N₅) |
| 58 | m/z = 917.19(C₆₃H₂₈D₁₅N₅S) |
| 59 | m/z = 810.05(C₅₇H₂₅D₁₅N₅) |
| 60 | m/z = 812.05(C₅₇H₂₇D₁₅N₅) |
| 61 | m/z = 676.89(C₄₈H₂₄D₁₀N₄) |
| 62 | m/z = 676.89(C₄₈H₂₄D₁₀N₄) |
| 63 | m/z = 677.89(C₄₈H₂₅D₁₀N₄) |
| 64 | m/z = 677.89(C₄₈H₂₅D₁₀N₄) |
| 65 | m/z = 678.89(C₄₈H₂₆D₁₀N₄) |
| 66 | m/z = 676.89(C₄₈H₂₄D₁₀N₄) |
| 67 | m/z = 755.99(C₅₄H₃₁D₁₀N₄) |
| 68 | m/z = 752.99(C₅₄H₂₈D₁₀N₄) |
| 69 | m/z = 843.07(C₆₀H₃₀D₁₀N₄O) |
| 70 | m/z = 753.99(C₅₄H₂₉D₁₀N₄) |
| 71 | m/z = 767.99(C₅₄H₂₉D₁₀N₅) |
| 72 | m/z = 754.99(C₅₄H₃₀D₁₀N₄) |
| 73 | m/z = 766.97(C₅₄H₂₆D₁₀N₄O) |
| 74 | m/z = 752.99(C₅₄H₂₈D₁₀N₄) |
| 75 | m/z = 830.09(C₆₀H₃₃D₁₀N₄) |
| 76 | m/z = 843.07(C₆₀H₃₀D₁₀N₄O) |
| 77 | m/z = 728.95(C₅₂H₂₈D₁₀N₄) |
| 78 | m/z = 843.07(C₆₀H₃₀D₁₀N₄O) |
| 79 | m/z = 726.95(C₅₂H₂₆D₁₀N₄) |
| 80 | m/z = 753.99(C₅₄H₂₉D₁₀N₄) |
| 81 | m/z = 666.87(C₄₅H₁₈D₁₀N₄S) |
| 82 | m/z = 666.87(C₄₅H₁₈D₁₀N₄S) |
| 83 | m/z = 668.87(C₄₅H₂₀D₁₀N₄S) |
| 84 | m/z = 667.87(C₄₅H₁₉D₁₀N₄S) |
| 85 | m/z = 666.87(C₄₅H₁₈D₁₀N₄S) |
| 86 | m/z = 667.87(C₄₅H₁₉D₁₀N₄S) |
| 87 | m/z = 742.97(C₅₁H₂₂D₁₀N₄S) |
| 88 | m/z = 742.97(C₅₁H₂₂D₁₀N₄S) |
| 89 | m/z = 759.95(C₅₁H₂₃D₁₀N₄SO) |
| 90 | m/z = 743.97(C₅₁H₂₃D₁₀N₄S) |
| 91 | m/z = 756.97(C₅₁H₂₂D₁₀N₅S) |
| 92 | m/z = 742.97(C₅₁H₂₂D₁₀N₄S) |
| 93 | m/z = 756.95(C₅₁H₂₀D₁₀N₄SO) |
| 94 | m/z = 744.97(C₅₁H₂₄D₁₀N₄S) |
| 95 | m/z = 819.06(C₅₇H₂₆D₁₀N₄S) |
| 96 | m/z = 776.01(C₅₁H₂₃D₁₀N₄S₂) |
| 97 | m/z = 766.99(C₅₃H₂₂D₁₀N₄S) |
| 98 | m/z = 833.05(C₅₇H₂₄D₁₀N₄SO) |
| 99 | m/z = 743.97(C₅₁H₂₃D₁₀N₄S) |
| 100 | m/z = 742.97(C₅₁H₂₂D₁₀N₄S) |
| 101 | m/z = 650.81(C₄₅H₁₈D₁₀N₄O) |
| 102 | m/z = 651.81(C₄₅H₁₈D₁₀N₄O) |
| 103 | m/z = 650.81(C₄₅H₁₈D₁₀N₄O) |
| 104 | m/z = 652.81(C₄₅H₂₀D₁₀N₄O) |
| 105 | m/z = 653.81(C₄₅H₂₁D₁₀N₄O) |
| 106 | m/z = 650.81(C₄₅H₁₈D₁₀N₄O) |
| 107 | m/z = 726.91(C₅₁H₂₂D₁₀N₄O) |
| 108 | m/z = 727.91(C₅₁H₂₃D₁₀N₄O) |
| 109 | m/z = 743.89(C₅₁H₂₃D₁₀N₄O₂) |
| 110 | m/z = 803.00(C₅₇H₂₆D₁₀N₄O) |
| 111 | m/z = 816.00(C₅₇H₂₅D₁₀N₅O) |
| 112 | m/z = 726.91(C₅₁H₂₂D₁₀N₄O) |
| 113 | m/z = 757.95(C₅₁H₂₁D₁₀N₄OS) |
| 114 | m/z = 728.91(C₅₁H₂₄D₁₀N₄O) |
| 115 | m/z = 805.00(C₅₇H₂₈D₁₀N₄O) |
| 116 | m/z = 833.05(C₅₇H₂₄D₁₀N₄OS) |
| 117 | m/z = 700.87(C₄₉H₂₀D₁₀N₄O) |
| 118 | m/z = 819.99(C₅₇H₂₇D₁₀N₄O₂) |
| 119 | m/z = 701.87(C₄₉H₂₁D₁₀N₄O) |
| 120 | m/z = 726.91(C₅₁H₂₂D₁₀N₄O) |
| 121 | m/z = 731.95(C₅₁H₁₉D₁₅N₅) |
| 122 | m/z = 732.95(C₅₁H₂₀D₁₅N₅) |
| 123 | m/z = 733.95(C₅₁H₂₁D₁₅N₅) |
| 124 | m/z = 731.95(C₅₁H₁₉D₁₅N₅) |
| 125 | m/z = 732.95(C₅₁H₂₀D₁₅N₅) |
| 126 | m/z = 733.95(C₅₁H₂₁D₁₅N₅) |
| 127 | m/z = 807.05(C₅₇H₂₂D₁₅N₅) |
| 128 | m/z = 808.05(C₅₇H₂₃D₁₅N₅) |
| 129 | m/z = 821.03(C₅₇H₂₀D₁₅N₅O) |
| 130 | m/z = 808.05(C₅₇H₂₃D₁₅N₅) |
| 131 | m/z = 823.05(C₅₇H₂₄D₁₅N₆) |
| 132 | m/z = 807.05(C₅₇H₂₂D₁₅N₅) |
| 133 | m/z = 823.03(C₅₇H₂₂D₁₅N₅O) |
| 134 | m/z = 808.05(C₅₇H₂₃D₁₅N₅) |
| 135 | m/z = 850.11(C₆₀H₂₉D₁₅N₄) |
| 136 | m/z = 842.09(C₅₇H₂₅D₁₅N₅OS) |
| 137 | m/z = 781.01(C₅₅H₂₀D₁₅N₅) |
| 138 | m/z = 898.13(C₆₃H₂₅D₁₅N₅O) |
| 139 | m/z = 808.05(C₅₇H₂₃D₁₅N₅) |
| 140 | m/z = 810.05(C₅₇H₂₅D₁₅N₅) |
| 141 | m/z = 676.89(C₄₈H₂₄D₁₀N₄) |
| 142 | m/z = 676.89(C₄₈H₂₄D₁₀N₄) |
| 143 | m/z = 678.89(C₄₈H₂₆D₁₀N₄) |
| 144 | m/z = 677.89(C₄₈H₂₅D₁₀N₄) |
| 145 | m/z = 676.89(C₄₈H₂₄D₁₀N₄) |
| 146 | m/z = 676.89(C₄₈H₂₄D₁₀N₄) |
| 147 | m/z = 754.99(C₅₄H₃₀D₁₀N₄) |
| 148 | m/z = 753.99(C₅₄H₂₉D₁₀N₄) |
| 149 | m/z = 844.07(C₆₀H₃₁D₁₀N₄O) |
| 150 | m/z = 752.99(C₅₄H₂₈D₁₀N₄) |
| 151 | m/z = 766.99(C₅₄H₂₈D₁₀N₅) |
| 152 | m/z = 755.99(C₅₄H₃₁D₁₀N₄) |
| 153 | m/z = 829.09(C₆₀H₃₂D₁₀N₄) |
| 154 | m/z = 752.99(C₅₄H₂₈D₁₀N₄) |
| 155 | m/z = 753.99(C₅₄H₂₉D₁₀N₄) |
| 156 | m/z = 766.97(C₅₄H₂₆D₁₀N₄O) |
| 157 | m/z = 728.95(C₅₂H₂₈D₁₀N₄) |
| 158 | m/z = 846.07(C₆₀H₃₃D₁₀N₄O) |
| 159 | m/z = 726.95(C₅₂H₂₆D₁₀N₄) |
| 160 | m/z = 753.99(C₅₄H₂₉D₁₀N₄) |
| 161 | m/z = 666.87(C₄₅H₁₈D₁₀N₄S) |
| 162 | m/z = 667.87(C₄₅H₁₉D₁₀N₄S) |
| 163 | m/z = 666.87(C₄₅H₁₈D₁₀N₄S) |
| 164 | m/z = 666.87(C₄₅H₁₈D₁₀N₄S) |
| 165 | m/z = 668.87(C₄₅H₂₀D₁₀N₄S) |
| 166 | m/z = 666.87(C₄₅H₁₈D₁₀N₄S) |
| 167 | m/z = 742.97(C₅₁H₂₂D₁₀N₄S) |
| 168 | m/z = 742.97(C₅₁H₂₂D₁₀N₄S) |
| 169 | m/z = 759.95(C₅₁H₂₃D₁₀N₄OS) |
| 170 | m/z = 742.97(C₅₁H₂₂D₁₀N₄S) |
| 171 | m/z = 756.97(C₅₁H₂₂D₁₀N₅S) |
| 172 | m/z = 742.97(C₅₁H₂₂D₁₀N₄S) |
| 173 | m/z = 756.95(C₅₁H₂₀D₁₀N₄OS) |
| 174 | m/z = 742.97(C₅₁H₂₂D₁₀N₄S) |
| 175 | m/z = 822.06(C₅₇H₂₉D₁₀N₄S) |
| 176 | m/z = 832.06(C₅₇H₂₅D₁₀N₅S) |
| 177 | m/z = 717.93(C₄₉H₂₁D₁₀N₄S) |
| 178 | m/z = 821.06(C₅₇H₂₈D₁₀N₄S) |
| 179 | m/z = 832.06(C₅₇H₂₅D₁₀N₅S) |
| 180 | m/z = 742.97(C₅₁H₂₂D₁₀N₄S) |
| 181 | m/z = 650.81(C₄₅H₁₈D₁₀N₄O) |
| 182 | m/z = 650.81(C₄₅H₁₈D₁₀N₄O) |
| 183 | m/z = 650.81(C₄₅H₁₈D₁₀N₄O) |
| 184 | m/z = 650.81(C₄₅H₁₈D₁₀N₄O) |
| 185 | m/z = 650.81(C₄₅H₁₈D₁₀N₄O) |
| 186 | m/z = 650.81(C₄₅H₁₈D₁₀N₄O) |
| 187 | m/z = 726.91(C₅₁H₂₂D₁₀N₄O) |
| 188 | m/z = 726.91(C₅₁H₂₂D₁₀N₄O) |
| 189 | m/z = 740.89(C₅₁H₂₀D₁₀N₄O₂) |
| 190 | m/z = 726.91(C₅₁H₂₂D₁₀N₄O) |
| 191 | m/z = 739.90(C₅₁H₂₁D₁₀N₅O) |
| 192 | m/z = 726.91(C₅₁H₂₂D₁₀N₄O) |
| 193 | m/z = 740.89(C₅₁H₂₀D₁₀N₄O₂) |
| 194 | m/z = 726.91(C₅₁H₂₂D₁₀N₄O) |
| 195 | m/z = 726.91(C₅₁H₂₂D₁₀N₄O) |
| 196 | m/z = 816.00(C₅₇H₂₅D₁₀N₅O) |
| 197 | m/z = 750.93(C₅₃H₂₂D₁₀N₄) |
| 198 | m/z = 816.99(C₅₇H₂₄D₁₀N₄O₂) |
| 199 | m/z = 700.87(C₄₉H₂₀D₁₀N₄O) |
| 200 | m/z = 726.91(C₅₁H₂₂D₁₀N₄O) |
| 201 | m/z = 730.95(C₅₁H₁₈D₁₅N₅) |
| 202 | m/z = 730.95(C₅₁H₁₈D₁₅N₅) |
| 203 | m/z = 730.95(C₅₁H₁₈D₁₅N₅) |
| 204 | m/z = 730.95(C₅₁H₁₈D₁₅N₅) |
| 205 | m/z = 730.95(C₅₁H₁₈D₁₅N₅) |
| 206 | m/z = 730.95(C₅₁H₁₈D₁₅N₅) |
| 207 | m/z = 807.05(C₅₇H₂₂D₁₅N₅) |
| 208 | m/z = 807.05(C₅₇H₂₂D₁₅N₅) |
| 209 | m/z = 897.13(C₆₃H₂₄D₁₅N₅O) |
| 210 | m/z = 807.05(C₅₇H₂₂D₁₅N₅) |
| 211 | m/z = 820.05(C₅₇H₂₁D₁₅N₆) |
| 212 | m/z = 807.05(C₅₇H₂₂D₁₅N₅) |
| 213 | m/z = 821.03(C₅₇H₂₀D₁₅N₅O) |
| 214 | m/z = 807.05(C₅₇H₂₂D₁₅N₅) |
| 215 | m/z = 807.05(C₅₇H₂₂D₁₅N₅) |
| 216 | m/z = 897.13(C₆₃H₂₄D₁₅N₅O) |
| 217 | m/z = 857.11(C₆₁H₂₄D₁₅N₅) |
| 218 | m/z = 883.15(C₆₃H₂₆D₁₅N₅) |
| 219 | m/z = 807.05(C₅₇H₂₂D₁₅N₅) |
| 220 | m/z = 807.05(C₅₇H₂₂D₁₅N₅) |
| 221 | m/z = 676.89(C₄₈H₂₄D₁₀N₄) |
| 222 | m/z = 676.89(C₄₈H₂₄D₁₀N₄) |
| 223 | m/z = 676.89(C₄₈H₂₄D₁₀N₄) |
| 224 | m/z = 676.89(C₄₈H₂₄D₁₀N₄) |
| 225 | m/z = 676.89(C₄₈H₂₄D₁₀N₄) |
| 226 | m/z = 676.89(C₄₈H₂₄D₁₀N₄) |
| 227 | m/z = 752.99(C₅₄H₂₈D₁₀N₄) |
| 228 | m/z = 752.99(C₅₄H₂₈D₁₀N₄) |
| 229 | m/z = 783.03(C₅₄H₂₆D₁₀N₄S) |
| 230 | m/z = 752.99(C₅₄H₂₈D₁₀N₄) |
| 231 | m/z = 765.99(C₅₄H₂₇D₁₀N₅) |
| 232 | m/z = 752.99(C₅₄H₂₈D₁₀N₄) |
| 233 | m/z = 766.97(C₅₄H₂₆D₁₀N₄O) |
| 234 | m/z = 752.99(C₅₄H₂₈D₁₀N₄) |
| 235 | m/z = 752.99(C₅₄H₂₈D₁₀N₄) |
| 236 | m/z = 842.08(C₆₀H₃₁D₁₀N₅) |
| 237 | m/z = 777.01(C₅₆H₂₆D₁₀N₄) |
| 238 | m/z = 843.07(C₆₀H₃₀D₁₀N₄O) |
| 239 | m/z = 726.95(C₅₂H₂₆D₁₀N₄) |
| 240 | m/z = 752.99(C₅₄H₂₈D₁₀N₄) |
| 241 | m/z = 666.87(C₄₅H₁₈D₁₀N₄S) |
| 242 | m/z = 666.87(C₄₅H₁₈D₁₀N₄S) |
| 243 | m/z = 666.87(C₄₅H₁₈D₁₀N₄S) |
| 244 | m/z = 666.87(C₄₅H₁₈D₁₀N₄S) |
| 245 | m/z = 666.87(C₄₅H₁₈D₁₀N₄S) |
| 246 | m/z = 666.87(C₄₅H₁₈D₁₀N₄S) |
| 247 | m/z = 742.97(C₅₁H₂₂D₁₀N₄S) |
| 248 | m/z = 742.97(C₅₁H₂₂D₁₀N₄S) |
| 249 | m/z = 756.95(C₅₁H₂₀D₁₀N₄OS) |
| 250 | m/z = 742.97(C₅₁H₂₂D₁₀N₄S) |
| 251 | m/z = 845.06(C₅₇H₂₄D₁₀N₆S) |
| 252 | m/z = 742.97(C₅₁H₂₂D₁₀N₄S) |
| 253 | m/z = 756.95(C₅₁H₂₀D₁₀N₄OS) |
| 254 | m/z = 742.97(C₅₁H₂₂D₁₀N₄S) |
| 255 | m/z = 742.97(C₅₁H₂₂D₁₀N₄S) |
| 256 | m/z = 783.03(C₅₄H₂₆D₁₀N₄S) |
| 257 | m/z = 716.93(C₄₉H₂₀D₁₀N₄S) |
| 258 | m/z = 755.97(C₅₁H₂₅D₁₀N₅S) |
| 259 | m/z = 716.93(C₄₉H₂₀D₁₀N₄S) |
| 260 | m/z = 742.97(C₅₁H₂₂D₁₀N₄S) |
| 261 | m/z = 730.95(C₅₁H₁₈D₁₅N₅) |
| 262 | m/z = 730.95(C₅₁H₁₈D₁₅N₅) |
| 263 | m/z = 730.95(C₅₁H₁₈D₁₅N₅) |
| 264 | m/z = 730.95(C₅₁H₁₈D₁₅N₅) |
| 265 | m/z = 730.95(C₅₁H₁₈D₁₅N₅) |
| 266 | m/z = 730.95(C₅₁H₁₈D₁₅N₅) |
| 267 | m/z = 807.05(C₅₇H₂₂D₁₅N₅) |
| 268 | m/z = 807.05(C₅₇H₂₂D₁₅N₅) |
| 269 | m/z = 897.13(C₆₃H₂₄D₁₅N₅O) |
| 270 | m/z = 807.05(C₅₇H₂₂D₁₅N₅) |
| 271 | m/z = 820.05(C₅₇H₂₁D₁₅N₆) |
| 272 | m/z = 807.05(C₅₇H₂₂D₁₅N₅) |
| 273 | m/z = 821.03(C₅₇H₂₀D₁₅N₅O) |
| 274 | m/z = 807.05(C₅₇H₂₂D₁₅N₅) |
| 275 | m/z = 807.05(C₅₇H₂₂D₁₅N₅) |
| 276 | m/z = 897.13(C₆₃H₂₄D₁₅N₅O) |
| 277 | m/z = 831.07(C₅₉H₂₂D₁₅N₅) |
| 278 | m/z = 913.19(C₆₃H₂₄D₁₅N₅OS) |
| 279 | m/z = 807.05(C₅₇H₂₂D₁₅N₅) |
| 280 | m/z = 807.05(C₅₇H₂₂D₁₅N₅) |
| 281 | m/z = 676.89(C₄₈H₂₄D₁₀N₄) |
| 282 | m/z = 676.89(C₄₈H₂₄D₁₀N₄) |
| 283 | m/z = 676.89(C₄₈H₂₄D₁₀N₄) |
| 284 | m/z = 676.89(C₄₈H₂₄D₁₀N₄) |
| 285 | m/z = 676.89(C₄₈H₂₄D₁₀N₄) |
| 286 | m/z = 676.89(C₄₈H₂₄D₁₀N₄) |
| 287 | m/z = 752.99(C₅₄H₂₈D₁₀N₄) |
| 288 | m/z = 752.99(C₅₄H₂₈D₁₀N₄) |
| 289 | m/z = 766.97(C₅₄H₂₆D₁₀N₄O) |
| 290 | m/z = 752.99(C₅₄H₂₈D₁₀N₄) |
| 291 | m/z = 855.08(C₆₀H₃₀D₁₀N₆) |
| 292 | m/z = 752.99(C₅₄H₂₈D₁₀N₄) |
| 293 | m/z = 766.97(C₅₄H₂₆D₁₀N₄O) |
| 294 | m/z = 752.99(C₅₄H₂₈D₁₀N₄) |
| 295 | m/z = 752.99(C₅₄H₂₈D₁₀N₄) |
| 296 | m/z = 843.07(C₆₀H₃₀D₁₀N₄O) |
| 297 | m/z = 726.95(C₅₂H₂₆D₁₀N₄) |
| 298 | m/z = 869.15(C₆₃H₃₆D₁₀N₄) |
| 299 | m/z = 726.95(C₅₂H₂₆D₁₀N₄) |
| 300 | m/z = 752.99(C₅₄H₂₈D₁₀N₄) |
| 301 | m/z = 666.87(C₄₅H₁₈D₁₀N₄S) |
| 302 | m/z = 666.87(C₄₅H₁₈D₁₀N₄S) |
| 303 | m/z = 666.87(C₄₅H₁₈D₁₀N₄S) |
| 304 | m/z = 666.87(C₄₅H₁₈D₁₀N₄S) |
| 305 | m/z = 666.87(C₄₅H₁₈D₁₀N₄S) |
| 306 | m/z = 666.87(C₄₅H₁₈D₁₀N₄S) |
| 307 | m/z = 742.97(C₅₁H₂₂D₁₀N₄S) |
| 308 | m/z = 742.97(C₅₁H₂₂D₁₀N₄S) |
| 309 | m/z = 756.95(C₅₁H₂₀D₁₀N₄OS) |
| 310 | m/z = 742.97(C₅₁H₂₂D₁₀N₄S) |
| 311 | m/z = 845.06(C₅₇H₂₄D₁₀N₆S) |
| 312 | m/z = 742.97(C₅₁H₂₂D₁₀N₄S) |
| 313 | m/z = 756.95(C₅₁H₂₀D₁₀N₄OS) |
| 314 | m/z = 742.97(C₅₁H₂₂D₁₀N₄S) |
| 315 | m/z = 742.97(C₅₁H₂₂D₁₀N₄S) |
| 316 | m/z = 783.03(C₅₄H₂₆D₁₀N₄S) |
| 317 | m/z = 817.05(C₅₁H₂₀D₁₀N₄OS) |
| 318 | m/z = 849.11(C₅₇H₂₄D₁₀N₄S₂) |
| 319 | m/z = 716.93(C₄₉H₂₀D₁₀N₄S) |
| 320 | m/z = 742.97(C₅₇H₂₄D₁₀N₄S) |
| 321 | m/z = 650.81(C₄₅H₁₈D₁₀N₄O) |
| 322 | m/z = 650.81(C₄₅H₁₈D₁₀N₄O) |
| 323 | m/z = 650.81(C₄₅H₁₈D₁₀N₄O) |
| 324 | m/z = 650.81(C₄₅H₁₈D₁₀N₄O) |
| 325 | m/z = 650.81(C₄₅H₁₈D₁₀N₄O) |
| 326 | m/z = 650.81(C₄₅H₁₈D₁₀N₄O) |
| 327 | m/z = 726.91(C₅₁H₂₂D₁₀N₄O) |
| 328 | m/z = 726.91(C₅₁H₂₂D₁₀N₄O) |
| 329 | m/z = 740.89(C₅₁H₂₀D₁₀N₄O₂) |
| 330 | m/z = 726.91(C₅₁H₂₂D₁₀N₄O) |
| 331 | m/z = 739.90(C₅₁H₂₁D₁₀N₅O) |
| 332 | m/z = 726.91(C₅₁H₂₂D₁₀N₄O) |
| 333 | m/z = 740.89(C₅₁H₂₀D₁₀N₄O₂) |
| 334 | m/z = 726.91(C₅₁H₂₂D₁₀N₄O) |
| 335 | m/z = 726.91(C₅₁H₂₂D₁₀N₄O) |
| 336 | m/z = 803.00(C₅₇H₂₆D₁₀N₄O) |
| 337 | m/z = 800.99(C₅₇H₂₄D₁₀N₄O) |
| 338 | m/z = 833.05(C₅₇H₂₄D₁₀N₄OS) |
| 339 | m/z = 700.87(C₄₉H₂₀D₁₀N₄O) |
| 340 | m/z = 740.89(C₅₁H₂₀D₁₀N₄O₂) |
| 341 | m/z = 730.95(C₅₁H₁₈D₁₅N₅) |
| 342 | m/z = 730.95(C₅₁H₁₈D₁₅N₅) |
| 343 | m/z = 730.95(C₅₁H₁₈D₁₅N₅) |
| 344 | m/z = 730.95(C₅₁H₁₈D₁₅N₅) |
| 345 | m/z = 730.95(C₅₁H₁₈D₁₅N₅) |
| 346 | m/z = 730.95(C₅₁H₁₈D₁₅N₅) |
| 347 | m/z = 807.05(C₅₇H₂₂D₁₅N₅) |
| 348 | m/z = 881.13(C₆₃H₂₄D₁₅N₅) |
| 349 | m/z = 897.13(C₆₃H₂₄D₁₅N₅O) |
| 350 | m/z = 807.05(C₅₇H₂₂D₁₅N₅) |
| 351 | m/z = 820.05(C₅₇H₂₁D₁₅N₆) |
| 352 | m/z = 807.05(C₅₇H₂₂D₁₅N₅) |
| 353 | m/z = 837.09(C₅₇H₂₀D₁₅N₅S) |
| 354 | m/z = 807.05(C₅₇H₂₂D₁₅N₅) |
| 355 | m/z = 807.05(C₅₇H₂₂D₁₅N₅) |
| 356 | m/z = 897.13(C₆₃H₂₄D₁₅N₅O) |
| 357 | m/z = 781.01(C₅₅H₂₀D₁₅N₅) |
| 358 | m/z = 913.19(C₆₃H₂₄D₁₅N₅S) |
| 359 | m/z = 807.05(C₅₇H₂₂D₁₅N₅) |
| 360 | m/z = 807.05(C₅₇H₂₂D₁₅N₅) |
| 361 | m/z = 676.89(C₄₈H₂₄D₁₀N₄) |
| 362 | m/z = 676.89(C₄₈H₂₄D₁₀N₄) |
| 363 | m/z = 676.89(C₄₈H₂₄D₁₀N₄) |
| 364 | m/z = 676.89(C₄₈H₂₄D₁₀N₄) |
| 365 | m/z = 675.89(C₄₈H₂₅D₉N₄) |
| 366 | m/z = 676.89(C₄₈H₂₄D₁₀N₄) |
| 367 | m/z = 752.99(C₅₄H₂₈D₁₀N₄) |
| 368 | m/z = 752.99(C₅₄H₂₈D₁₀N₄) |
| 369 | m/z = 766.97(C₅₄H₂₆D₁₀N₄O) |
| 370 | m/z = 750.99(C₅₄H₃₀D₈N₄) |
| 371 | m/z = 853.08(C₆₀H₃₂D₈N₆) |
| 372 | m/z = 749.99(C₅₄H₃₁D₇N₄) |
| 373 | m/z = 766.97(C₅₄H₂₆D₁₀N₄O) |
| 374 | m/z = 752.99(C₅₄H₂₈D₁₀N₄) |
| 375 | m/z = 749.99(C₅₄H₃₁D₇N₄) |
| 376 | m/z = 829.09(C₆₀H₃₂D₁₀N₄) |
| 377 | m/z = 826.07(C₆₀H₃₁D₉N₄) |
| 378 | m/z = 829.09(C₆₀H₃₂D₁₀N₄) |
| 379 | m/z = 726.95(C₅₂H₂₆D₁₀N₄) |
| 380 | m/z = 752.99(C₅₄H₂₈D₁₀N₄) |
| 381 | m/z = 682.93(C₄₈H₁₈D₁₆N₄) |
| 382 | m/z = 680.91(C₄₈H₂₀D₁₄N₄) |
| 383 | m/z = 680.92(C₄₈H₁₈D₁₅N₄) |
| 384 | m/z = 683.93(C₄₈H₁₇D₁₇N₄) |
| 385 | m/z = 680.92(C₄₈H₁₈D₁₅N₄) |
| 386 | m/z = 680.92(C₄₈H₁₈D₁₅N₄) |
| 387 | m/z = 680.91(C₄₈H₂₀D₁₄N₄) |
| 388 | m/z = 762.04(C₅₄H₁₉D₁₉N₄) |
| 389 | m/z = 760.03(C₅₄H₂₁D₁₇N₄) |
| 390 | m/z = 762.04(C₅₄H₁₉D₁₉N₄) |
| 391 | m/z = 733.97(C₅₁H₁₅D₁₈N₅) |
| 392 | m/z = 735.98(C₅₁H₁₃D₂₀N₅) |
| 393 | m/z = 735.98(C₅₁H₁₃D₂₀N₅) |
| 394 | m/z = 733.97(C₅₁H₁₅D₁₈N₅) |
| 395 | m/z = 736.99(C₅₁H₁₂D₂₁N₅) |
| 396 | m/z = 817.11(C₅₇H₁₂D₂₅N₅) |
| 397 | m/z = 812.08(C₅₇H₁₇D₂₀N₅) |
| 398 | m/z = 817.11(C₅₇H₁₂D₂₅N₅) |
| 399 | m/z = 816.10(C₅₇H₁₃D₂₄N₅) |
| 400 | m/z = 822.04(C₅₇H₁₉D₁₆N₅) |
| 401 | m/z = 672.90(C₄₅H₁₂D₁₆N₄S) |
| 402 | m/z = 671.90(C₄₅H₁₃D₁₅N₄S) |
| 403 | m/z = 671.90(C₄₅H₁₃D₁₅N₄S) |
| 404 | m/z = 673.90(C₄₅H₁₁D₁₇N₄S) |
| 405 | m/z = 674.92(C₄₅H₁₀D₁₈N₄S) |
| 406 | m/z = 753.03(C₅₁H₁₂D₂₀N₄S) |
| 407 | m/z = 673.90(C₄₅H₁₁D₁₇N₄S) |
| 408 | m/z = 671.90(C₄₅H₁₃D₁₅N₄S) |
| 409 | m/z = 671.90(C₄₅H₁₃D₁₅N₄S) |
| 410 | m/z = 673.90(C₄₅H₁₁D₁₇N₄S) |
| 411 | m/z = 842.10(C₅₇H₁₅D₁₉N₄OS) |
| 412 | m/z = 655.84(C₄₅H₁₃D₁₅N₄O) |
| 413 | m/z = 655.84(C₄₅H₁₃D₁₅N₄O) |
| 414 | m/z = 657.85(C₄₅H₁₁D₁₇N₄O) |
| 415 | m/z = 657.85(C₄₅H₁₁D₁₇N₄O) |
| 416 | m/z = 736.97(C₅₁H₁₂D₂₀N₄O) |
| 417 | m/z = 734.96(C₅₁H₁₄D₁₈N₄O) |
| 418 | m/z = 747.95(C₅₁H₁₃D₁₈N₅O) |
| 419 | m/z = 655.84(C₄₅H₁₃D₁₅N₄O) |
| 420 | m/z = 828.05(C₅₇H₁₃D₂₁N₄O₂) |
| 421 | m/z = 682.93(C₄₈H₁₈D₁₆N₄) |
| 422 | m/z = 681.92(C₄₈H₁₉D₁₅N₄) |
| 423 | m/z = 681.92(C₄₈H₁₉D₁₅N₄) |
| 424 | m/z = 683.93(C₄₈H₁₇D₁₇N₄) |
| 425 | m/z = 684.93(C₄₈H₁₆D₁₈N₄) |
| 426 | m/z = 762.04(C₅₄H₁₉D₁₉N₄) |
| 427 | m/z = 760.03(C₅₄H₂₁D₁₇N₄) |
| 428 | m/z = 775.02(C₅₄H₁₈D₁₈N₄O) |
| 429 | m/z = 758.02(C₅₄H₂₃D₁₅N₄) |
| 430 | m/z = 764.04(C₅₄H₁₇D₂₁N₄) |
| 431 | m/z = 826.09(C₅₇H₁₄D₂₁N₅O) |
| 432 | m/z = 735.98(C₅₁H₁₃D₂₀N₅) |
| 433 | m/z = 736.99(C₅₁H₁₂D₂₁N₅) |
| 434 | m/z = 735.98(C₅₁H₁₃D₂₀N₅) |
| 435 | m/z = 734.98(C₅₁H₁₄D₁₉N₅) |
| 436 | m/z = 816.10(C₅₇H₁₃D₂₄N₅) |
| 437 | m/z = 817.11(C₅₇H₁₂D₂₅N₅) |
| 438 | m/z = 823.07(C₅₇H₁₈D₁₈N₆) |
| 439 | m/z = 810.07(C₅₇H₁₉D₁₈N₅) |
| 440 | m/z = 827.07(C₅₇H₁₄D₂₁N₅O) |
| 441 | m/z = 742.97(C₅₁H₂₂D₁₀N₄S) |
| 442 | m/z = 742.97(C₅₁H₂₂D₁₀N₄S) |
| 443 | m/z = 832.06(C₅₇H₂₅D₁₀N₄S) |
| 444 | m/z = 741.96(C₅₁H₂₃D₉N₄S) |
| 445 | m/z = 742.97(C₅₁H₂₂D₁₀N₄S) |
| 446 | m/z = 756.97(C₅₁H₂₂D₁₀N₅S) |
| 447 | m/z = 726.91(C₅₁H₂₂D₁₀N₄O) |
| 448 | m/z = 648.81(C₄₅H₂₀D₈N₄O) |
| 449 | m/z = 726.91(C₅₁H₂₂D₁₀N₄O) |
| 450 | m/z = 803.00(C₅₇H₂₆D₁₀N₄O) |
| 451 | m/z = 739.88(C₅₁H₂₁D₉N₄O₂) |
| 452 | m/z = 726.91(C₅₁H₂₂D₁₀N₄O) |
| 453 | m/z = 816.99(C₅₇H₂₄D₁₀N₄O₂) |
| 454 | m/z = 650.81(C₄₅H₁₈D₁₀N₄O) |
| 455 | m/z = 832.04(C₅₇H₂₅D₉N₄O₂) |
| 456 | m/z = 742.97(C₅₁H₂₂D₁₀N₄S) |
| 457 | m/z = 832.06(C₅₇H₂₅D₁₀N₅S) |
| 458 | m/z = 756.97(C₅₁H₂₀D₁₀N₄OS) |
| 459 | m/z = 755.97(C₅₁H₂₁D₉N₄OS) |
| 460 | m/z = 819.06(C₅₇H₂₆D₁₀N₄S) |
| 461 | m/z = 802.02(C₅₇H₂₇D₁₀N₅) |
| 462 | m/z = 804.03(C₅₇H₂₅D₁₂N₅) |
| 463 | m/z = 819.04(C₅₇H₂₂D₁₄N₆) |
| 464 | m/z = 802.02(C₅₇H₂₇D₁₀N₅) |
| 465 | m/z = 821.03(C₅₇H₂₀D₁₅N₅O) |
| 466 | m/z = 896.13(C₆₃H₂₅D₁₄N₅O) |
| 467 | m/z = 806.04(C₅₇H₂₃D₁₄N₅) |
| 468 | m/z = 730.95(C₅₁H₁₈D₁₅N₅) |
| 469 | m/z = 728.94(C₅₁H₂₀D₁₃N₅) |
| 470 | m/z = 881.14(C₆₃H₂₈D₁₃N₅) |
| 471 | m/z = 802.02(C₅₇H₂₇D₁₀N₅) |
| 472 | m/z = 806.04(C₅₇H₂₃D₁₄N₅) |
| 473 | m/z = 817.03(C₅₇H₂₄D₁₂N₆) |
| 474 | m/z = 728.94(C₅₁H₂₀D₁₃N₅) |
| 475 | m/z = 821.03(C₅₇H₂₀D₁₅N₅O) |
| 476 | m/z = 826.06(C₅₇H₁₅D₂₀N₅O) |
| 477 | m/z = 891.20(C₆₃H₁₈D₂₃N₅) |
| 478 | m/z = 735.98(C₅₁H₁₃D₂₀N₅) |
| 479 | m/z = 916.21(C₆₃H₂₁D₁₈N₅S) |
| 480 | m/z = 901.15(C₆₃H₂₀D₁₉N₅O) |
| 481 | m/z = 744.98(C₅₁H₂₀D₁₂N₄S) |
| 482 | m/z = 748.00(C₅₁H₁₇D₁₅N₄S) |
| 483 | m/z = 759.99(C₅₁H₁₇D₁₄N₅S) |
| 484 | m/z = 743.97(C₅₁H₂₁D₁₁N₄S) |
| 485 | m/z = 761.98(C₅₁H₁₅D₁₅N₄OS) |
| 486 | m/z = 751.01(C₅₁H₁₄D₁₈N₄S) |
| 487 | m/z = 731.94(C₅₁H₁₇D₁₅N₄O) |
| 488 | m/z = 726.91(C₅₁H₂₂D₁₀N₄O) |
| 489 | m/z = 761.98(C₅₁H₁₅D₁₅N₄OS) |
| 490 | m/z = 807.03(C₅₇H₂₂D₁₄N₄O) |
| 491 | m/z = 735.96(C₅₁H₁₃D₁₉N₄O) |
| 492 | m/z = 746.92(C₅₁H₁₄D₁₆N₄O₂) |
| 493 | m/z = 842.10(C₅₇H₁₅D₁₉N₄OS) |
| 494 | m/z = 809.04(C₅₇H₂₀D₁₆N₄O) |
| 495 | m/z = 735.96(C₅₁H₁₃D₁₉N₄O) |
| 496 | m/z = 840.09(C₅₇H₁₇D₁₇N₄OS) |
| 497 | m/z = 751.01(C₅₁H₁₄D₁₈N₄S) |
| 498 | m/z = 762.99(C₅₁H₁₄D₁₆N₄OS) |
| 499 | m/z = 821.07(C₅₇H₂₀D₁₄N₄S) |
| 500 | m/z = 916.19(C₆₃H₂₁D₁₇N₄OS) |
| 501 | m/z = 832.04(C₅₇H₂₅D₉N₄S) |
| 502 | m/z = 818.06(C₅₇H₂₇D₉N₄S) |
| 503 | m/z = 742.97(C₅₁H₂₂D₁₀N₄S) |
| 504 | m/z = 832.06(C₅₇H₂₅D₁₀N₅S) |
| 505 | m/z = 664.86(C₄₅H₂₀D₈N₄S) |
| 506 | m/z = 742.97(C₅₁H₂₂D₁₀N₄S) |
| 507 | m/z = 740.89(C₅₁H₂₀D₁₀N₄O₂) |
| 508 | m/z = 803.00(C₅₇H₂₆D₁₀N₄O) |
| 509 | m/z = 650.81(C₄₅H₁₈D₁₀N₄O) |
| 510 | m/z = 724.89(C₅₁H₂₄D₈N₄O) |
| 511 | m/z = 893.08(C₆₃H₂₈D₁₀N₄O₂) |
| 512 | m/z = 725.90(C₅₁H₂₃D₉N₄O) |
| 513 | m/z = 725.90(C₅₁H₂₃D₉N₄O) |
| 514 | m/z = 803.00(C₅₇H₂₆D₁₀N₄O) |
| 515 | m/z = 877.09(C₆₃H₂₈D₁₀N₄O) |
| 516 | m/z = 816.04(C₅₇H₂₅D₉N₄S) |
| 517 | m/z = 849.11(C₅₇H₂₄D₁₀N₄S₂) |
| 518 | m/z = 742.97(C₅₁H₂₂D₁₀N₄S) |
| 519 | m/z = 819.09(C₅₇H₂₆D₁₀N₄S) |
| 520 | m/z = 742.97(C₅₁H₂₂D₁₀N₄S) |
| 521 | m/z = 831.30(C₅₇H₂₅D₉N₄OS) |
| 522 | m/z = 819.09(C₅₇H₂₆D₁₀N₄S) |
| 523 | m/z = 742.97(C₅₁H₂₂D₁₀N₄S) |
| 524 | m/z = 832.06(C₅₇H₂₅D₁₀N₅S) |
| 525 | m/z = 664.86(C₄₅H₂₀D₈N₄S) |
| 526 | m/z = 742.97(C₅₁H₂₂D₁₀N₄S) |
| 527 | m/z = 740.30(C₅₁H₂₀D₁₀N₄S₂) |
| 528 | m/z = 802.35(C₅₇H₂₆D₁₀N₄O) |
| 529 | m/z = 650.29(C₄₅H₁₈D₁₀N₄O) |
| 530 | m/z = 724.89(C₅₁H₂₄D₈N₄O) |
| 531 | m/z = 892.36(C₆₃H₂₈D₁₀N₄O₂) |
| 532 | m/z = 725.90(C₅₁H₂₃D₉N₄O) |
| 533 | m/z = 725.90(C₅₁H₂₃D₉N₄O) |
| 534 | m/z = 802.35(C₅₇H₂₆D₁₀N₄O) |
| 535 | m/z = 877.09(C₆₃H₂₈D₁₀N₄O) |
| 536 | m/z = 816.04(C₅₇H₂₅D₉N₄S) |
| 537 | m/z = 849.11(C₅₇H₂₄D₁₀N₄S₂) |
| 538 | m/z = 742.97(C₅₁H₂₂D₁₀N₄S) |
| 539 | m/z = 819.09(C₅₇H₂₆D₁₀N₄S) |
| 540 | m/z = 742.97(C₅₁H₂₂D₁₀N₄S) |
| 541 | m/z = 742.97(C₅₁H₂₂D₁₀N₄S) |
| 542 | m/z = 819.09(C₅₇H₂₆D₁₀N₄S) |
| 543 | m/z = 746.99(C₅₁H₁₈D₁₄N₄S) |
| 544 | m/z = 743.97(C₅₁H₂₁D₁₁N₄S) |
| 545 | m/z = 739.95(C₅₁H₂₅D₇N₄S) |
| 546 | m/z = 728.92(C₅₁H₂₀D₁₂N₄O) |
| 547 | m/z = 805.02(C₅₇H₂₄D₁₂N₄O) |
| 548 | m/z = 804.02(C₅₇H₂₅D₁₁N₄O) |
| 549 | m/z = 725.30(C₅₁H₂₃D₉N₄O) |
| 550 | m/z = 805.02(C₅₇H₂₄D₁₂N₄O) |
| 551 | m/z = 920.43(C₆₆H₃₂D₁₂N₄O) |
| 552 | m/z = 830.42(C₆₀H₃₀D₁₂N₄) |
| 553 | m/z = 753.99(C₅₄H₂₇D₁₁N₄) |
| 554 | m/z = 830.42(C₆₀H₃₀D₁₂N₄) |
| 555 | m/z = 754.38(C₅₄H₂₆D₁₂N₄) |
| 556 | m/z = 880.42(C₆₃H₂₈D₁₃N₅) |
| 557 | m/z = 909.37(C₆₃H₂₇D₁₂N₅S) |
| 558 | m/z = 880.42(C₆₃H₂₈D₁₃N₅) |
| 559 | m/z = 806.40(C₅₇H₂₂D₁₅N₅) |
| 560 | m/z = 807.41(C₅₇H₂₂D₁₆N₅) |

TABLE 11

| Compound | FD = MS |
|---|---|
| 2-1 | m/z = 484.60(C₃₆H₂₄N₂) |
| 2-2 | m/z = 560.70(C₄₂H₂₈N₂) |
| 2-3 | m/z = 560.70(C₄₂H₂₈N₂) |
| 2-4 | m/z = 636.80(C₄₈H₃₂N₂) |
| 2-5 | m/z = 636.80(C₄₈H₃₂N₂) |
| 2-6 | m/z = 636.80(C₄₈H₃₂N₂) |
| 2-7 | m/z = 634.78(C₄₈H₃₀N₂) |
| 2-8 | m/z = 636.80(C₄₈H₃₂N₂) |
| 2-9 | m/z = 575.68(C₄₂H₂₆N₂) |
| 2-10 | m/z = 650.78(C₄₈H₃₀N₂O) |
| 2-11 | m/z = 636.80(C₄₈H₃₂N₂) |
| 2-12 | m/z = 636.80(C₄₈H₃₂N₂) |
| 2-13 | m/z = 712.90(C₅₄H₃₆N₂) |
| 2-14 | m/z = 650.78(C₄₈H₃₀N₂O) |
| 2-15 | m/z = 712.90(C₅₄H₃₆N₂) |
| 2-16 | m/z = 712.90(C₅₄H₃₆N₂) |
| 2-17 | m/z = 636.80(C₄₈H₃₂N₂) |
| 2-18 | m/z = 650.78(C₄₈H₃₀N₂O) |
| 2-19 | m/z = 712.90(C₅₄H₃₆N₂) |
| 2-20 | m/z = 710.88(C₅₄H₃₄N₂) |
| 2-21 | m/z = 636.80(C₄₈H₃₂N₂) |
| 2-22 | m/z = 636.80(C₄₈H₃₂N₂) |
| 2-23 | m/z = 636.80(C₄₈H₃₂N₂) |
| 2-24 | m/z = 590.74(C₄₂H₂₆N₂S) |
| 2-25 | m/z = 788.99(C₆₀H₄₀N₂) |
| 2-26 | m/z = 712.90(C₅₄H₃₆N₂) |
| 2-27 | m/z = 788.99(C₆₀H₄₀N₂) |
| 2-28 | m/z = 712.90(C₅₄H₃₆N₂) |
| 2-29 | m/z = 788.99(C₆₀H₄₀N₂) |
| 2-30 | m/z = 712.90(C₅₄H₃₆N₂) |
| 2-31 | m/z = 726.88(C₅₄H₃₄N₂O) |
| 2-32 | m/z = 560.70(C₄₂H₂₈N₂) |
| 2-33 | m/z = 636.80(C₄₈H₃₂N₂) |
| 2-34 | m/z = 636.80(C₄₈H₃₂N₂) |
| 2-35 | m/z = 712.90(C₅₄H₃₆N₂) |
| 2-36 | m/z = 636.80(C₄₈H₃₂N₂) |
| 2-37 | m/z = 712.90(C₅₄H₃₆N₂) |
| 2-38 | m/z = 712.90(C₅₄H₃₆N₂) |
| 2-39 | m/z = 574.68(C₄₂H₂₆N₂O) |
| 2-40 | m/z = 788.99(C₆₀H₄₀N₂) |
| 2-41 | m/z = 508.75(C₃₆D₂₄N₂) |
| 2-42 | m/z = 587.86(C₄₂HD₂₇N₂) |
| 2-43 | m/z = 588.75(C₄₂D₂₈N₂) |
| 2-44 | m/z = 666.98(C₄₈H₂D₃₀N₂) |
| 2-45 | m/z = 668.98(C₄₈D₃₂N₂) |
| 2-46 | m/z = 665.98(C₄₈H₃D₂₉N₂) |
| 2-47 | m/z = 662.98(C₄₈H₂D₂₈N₂) |
| 2-48 | m/z = 668.98(C₄₈D₃₂N₂) |
| 2-49 | m/z = 600.84(C₄₂D₂₆N₂O) |
| 2-50 | m/z = 678.98(C₄₈H₂D₂₈N₂) |
| 2-51 | m/z = 668.98(C₄₈D₃₂N₂) |
| 2-52 | m/z = 667.98(C₄₈HD₃₁N₂) |
| 2-53 | m/z = 748.12(C₅₄HD₃₅N₂) |
| 2-54 | m/z = 680.96(C₄₈D₃₀N₂O) |
| 2-55 | m/z = 747.10(C₅₄H₂D₃₄N₂) |
| 2-56 | m/z = 739.05(C₅₄H₁₀D₂₆N₂) |
| 2-57 | m/z = 668.98(C₄₈D₃₂N₂) |
| 2-58 | m/z = 677.98(C₄₈H₃D₂₇N₂O) |
| 2-59 | m/z = 746.10(C₅₄H₃D₃₃N₂) |
| 2-60 | m/z = 744.08(C₅₄HD₃₃N₂) |
| 2-61 | m/z = 668.98(C₄₈D₃₂N₂) |
| 2-62 | m/z = 666.98(C₄₈H₂D₃₀N₂) |
| 2-63 | m/z = 667.98(C₄₈HD₃₁N₂) |
| 2-64 | m/z = 614.89(C₄₂H₂D₂₄N₂S) |
| 2-65 | m/z = 826.22(C₆₀H₃D₃₇N₂) |
| 2-66 | m/z = 747.10(C₅₄H₂D₃₄N₂) |
| 2-67 | m/z = 829.24(C₆₀D₄₀N₂) |
| 2-68 | m/z = 746.10(C₅₄H₃D₃₃N₂) |
| 2-69 | m/z = 829.24(C₆₀D₄₀N₂) |
| 2-70 | m/z = 747.10(C₅₄H₂D₃₄N₂) |
| 2-71 | m/z = 759.09(C₅₄H₂D₃₂N₂) |
| 2-72 | m/z = 588.75(C₄₂D₂₈N₂) |
| 2-73 | m/z = 668.98(C₄₈D₃₂N₂) |
| 2-74 | m/z = 668.98(C₄₈D₃₂N₂) |
| 2-75 | m/z = 749.12(C₅₄D₃₆N₂) |
| 2-76 | m/z = 666.98(C₄₈H₂D₃₀N₂) |
| 2-77 | m/z = 824.21(C₆₀H₅D₃₅N₂) |
| 2-78 | m/z = 829.24(C₆₀D₄₀N₂) |
| 2-79 | m/z = 598.83(C₄₂H₂D₂₄N₂) |
| 2-80 | m/z = 862.22(C₆₀H₃D₃₇N₂) |
| 2-81 | m/z = 484.60(C₃₆H₂₄N₂) |
| 2-82 | m/z = 560.70(C₄₂H₂₈N₂) |
| 2-83 | m/z = 560.70(C₄₂H₂₈N₂) |
| 2-84 | m/z = 560.70(C₄₂H₂₈N₂) |
| 2-85 | m/z = 560.70(C₄₂H₂₈N₂) |
| 2-86 | m/z = 560.70(C₄₂H₂₈N₂) |
| 2-87 | m/z = 636.80(C₄₈H₃₂N₂) |
| 2-88 | m/z = 636.80(C₄₈H₃₂N₂) |
| 2-89 | m/z = 636.80(C₄₈H₃₂N₂) |
| 2-90 | m/z = 636.80(C₄₈H₃₂N₂) |
| 2-91 | m/z = 560.70(C₄₂H₂₈N₂) |
| 2-92 | m/z = 636.80(C₄₈H₃₂N₂) |
| 2-93 | m/z = 636.80(C₄₈H₃₂N₂) |
| 2-94 | m/z = 636.80(C₄₈H₃₂N₂) |
| 2-95 | m/z = 636.80(C₄₈H₃₂N₂) |
| 2-96 | m/z = 636.80(C₄₈H₃₂N₂) |
| 2-97 | m/z = 558.68(C₄₂H₂₆N₂) |
| 2-98 | m/z = 712.90(C₅₄H₃₆N₂) |
| 2-99 | m/z = 498.58(C₃₆H₂₂N₂O) |
| 2-100 | m/z = 712.90(C₅₄H₃₆N₂) |
| 2-101 | m/z = 484.60(C₃₆H₂₄N₂) |
| 2-102 | m/z = 560.70(C₄₂H₂₈N₂) |
| 2-103 | m/z = 498.58(C₃₆H₂₂N₂O) |
| 2-104 | m/z = 560.70(C₄₂H₂₈N₂) |
| 2-105 | m/z = 558.68(C₄₂H₂₆N₂) |
| 2-106 | m/z = 560.70(C₄₂H₂₈N₂) |
| 2-107 | m/z = 636.80(C₄₈H₃₂N₂) |
| 2-108 | m/z = 712.90(C₅₄H₃₆N₂) |
| 2-109 | m/z = 636.80(C₄₈H₃₂N₂) |
| 2-110 | m/z = 636.80(C₄₈H₃₂N₂) |
| 2-111 | m/z = 560.70(C₄₂H₂₈N₂) |
| 2-112 | m/z = 636.80(C₄₈H₃₂N₂) |
| 2-113 | m/z = 636.80(C₄₈H₃₂N₂) |
| 2-114 | m/z = 560.70(C₄₂H₂₈N₂) |
| 2-115 | m/z = 574.68(C₄₂H₂₆N₂O) |
| 2-116 | m/z = 484.60(C₃₆H₂₄N₂) |
| 2-117 | m/z = 560.70(C₄₂H₂₈N₂) |
| 2-118 | m/z = 560.70(C₄₂H₂₈N₂) |
| 2-119 | m/z = 560.70(C₄₂H₂₈N₂) |
| 2-120 | m/z = 636.80(C₄₈H₃₂N₂) |
| 2-121 | m/z = 508.75(C₃₆D₂₄N₂) |
| 2-122 | m/z = 588.87(C₄₂D₂₈N₂) |
| 2-123 | m/z = 588.87(C₄₂D₂₈N₂) |
| 2-124 | m/z = 586.86(C₄₂H₂D₂₆N₂) |
| 2-125 | m/z = 588.87(C₄₂D₂₈N₂) |
| 2-126 | m/z = 588.87(C₄₂D₂₈N₂) |
| 2-127 | m/z = 588.87(C₄₂D₂₈N₂) |
| 2-128 | m/z = 666.98(C₄₈H₂D₃₀N₂) |
| 2-129 | m/z = 665.98(C₄₈H₃D₂₉N₂) |
| 2-130 | m/z = 668.98(C₄₈D₃₂N₂) |
| 2-131 | m/z = 588.87(C₄₂D₂₈N₂) |
| 2-132 | m/z = 664.98(C₄₈H₄D₂₈N₂) |
| 2-133 | m/z = 663.96(C₄₈H₅D₂₇N₂) |
| 2-134 | m/z = 668.98(C₄₈D₃₂N₂) |
| 2-135 | m/z = 667.98(C₄₈HD₃₁N₂) |
| 2-136 | m/z = 585.87(C₄₂H₃D₂₅N₂) |
| 2-137 | m/z = 582.83(C₄₂H₂D₂₄N₂) |
| 2-138 | m/z = 749.12(C₅₄D₃₆N₂) |
| 2-139 | m/z = 518.71(C₃₆H₂D₂₀N₂O) |
| 2-140 | m/z = 748.12(C₅₄HD₃₅N₂) |
| 2-141 | m/z = 508.75(C₃₆D₂₄N₂) |
| 2-142 | m/z = 588.87(C₄₂D₂₈N₂) |
| 2-143 | m/z = 520.72(C₃₆D₂₂N₂O) |
| 2-144 | m/z = 583.84(C₄₂H₅D₂₃N₂) |
| 2-145 | m/z = 583.84(C₄₂HD₂₅N₂) |
| 2-146 | m/z = 588.87(C₄₂D₂₈N₂) |
| 2-147 | m/z = 668.98(C₄₈D₃₂N₂) |
| 2-148 | m/z = 746.12(C₅₄H₃D₃₃N₂) |
| 2-149 | m/z = 665.97(C₄₈H₃D₂₉N₂) |
| 2-150 | m/z = 668.98(C₄₈D₃₂N₂) |
| 2-151 | m/z = 587.87(C₄₂HD₂₇N₂) |
| 2-152 | m/z = 668.98(C₄₈D₃₂N₂) |
| 2-153 | m/z = 666.98(C₄₈H₂D₃₀N₂) |
| 2-154 | m/z = 588.87(C₄₂D₂₈N₂) |
| 2-155 | m/z = 596.82(C₄₂H₄D₂₂N₂O) |
| 2-156 | m/z = 508.75(C₃₆D₂₄N₂) |
| 2-157 | m/z = 588.87(C₄₂D₂₈N₂) |
| 2-158 | m/z = 586.86(C₄₂H₂D₂₆N₂) |
| 2-159 | m/z = 588.87(C₄₂D₂₈N₂) |
| 2-160 | m/z = 668.99(C₄₈D₃₂N₂) |

### Experimental Example 1

### 1) Manufacture of Organic Light Emitting Device

A glass substrate, in which ITO was thinly coated to have a thickness of 1,500 Å, was ultrasonically washed with distilled water. When the washing with distilled water is finished, the glass substrate was ultrasonically washed with a solvent such as acetone, methanol, and isopropyl alcohol, was dried and then was subjected to UVO treatment for 5 minutes by using UV in a UV washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

As the common layers, the hole injection layer 4,4′,4″-tris[2-naphthyl (phenyl)amino]triphenylamine (2-TNATA) and the hole transport layer N,N′-di(1-naphthyl)-N,N′-diphenyl-(1,1′-biphenyl)-4,4′-diamine (NPB) were formed on the ITO transparent electrode (positive electrode).

A light emitting layer was thermally vacuum deposited thereon as follows. The light emitting layer was deposited to have a thickness of 360 Å by using a heterocyclic compound of Chemical Formula 1 or Comparative Example compound described in the following Table 12 as a host and tris(2-phenylpyridine) iridium (Ir(ppy)₃) as a green phosphorescent dopant to dope the host with Ir(ppy)₃ in an amount of 7%. Thereafter, BCP as a hole blocking layer was deposited to have a thickness of 60 Å, and Alq₃ as an electron transport layer was deposited to have a thickness of 200 Å thereon. Finally, lithium fluoride (LiF) was deposited to have a thickness of 10 Å on the electron transport layer to form an electron injection layer, and then an aluminum (Al) negative electrode was deposited to have a thickness of 1200 Å on the electron injection layer to form a negative electrode, thereby manufacturing an organic electroluminescence device.

Meanwhile, all the organic compounds required for manufacturing an OLED device were subjected to vacuum sublimed purification under 10-8 to 10-6 torr for each material, and used for the manufacture of OLED.

Compounds Ref. 1 to Ref. 12 used as the Comparative Example compounds are as follows.

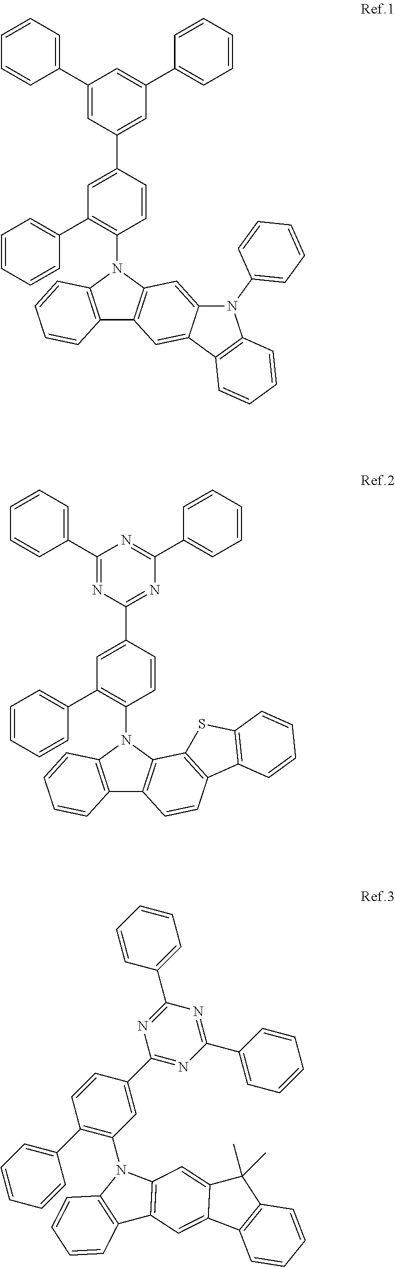 [SMILES: c1ccc(-c2cc(-c3ccccc3)cc(-c3ccc(-n4c5ccccc5c5cc6c7ccccc7n(-c7ccccc7)c6cc54)c(-c4ccccc4)c3)c2)cc1 ; c1ccc(-c2nc(-c3ccccc3)nc(-c3ccc(-n4c5ccccc5c5ccc6c7ccccc7sc6c54)c(-c4ccccc4)c3)n2)cc1 ; CC1(C)c2ccccc2-c2cc3c4ccccc4n(-c4cc(-c5nc(-c6ccccc6)nc(-c6ccccc6)n5)ccc4-c4ccccc4)c3cc21]

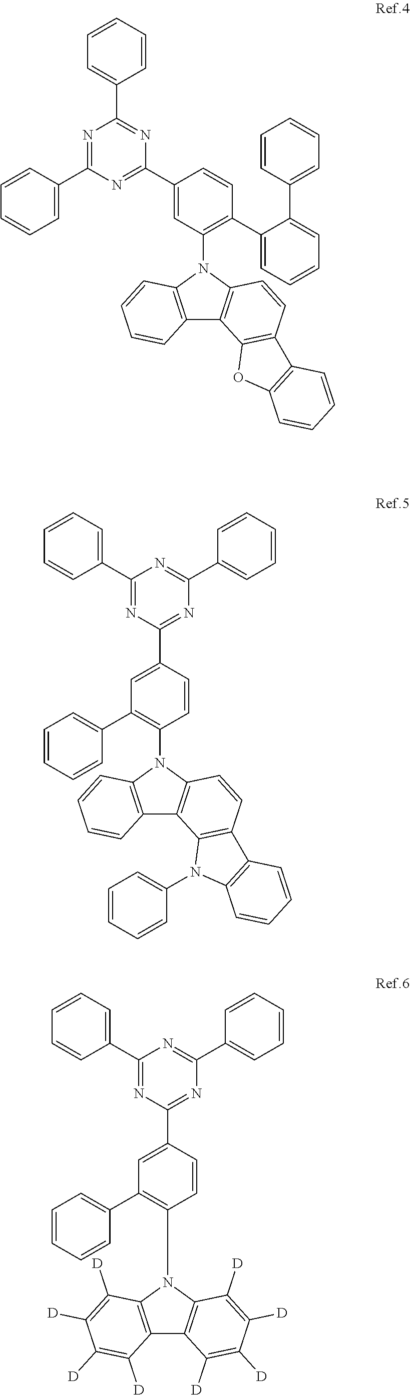 [SMILES: c1ccc(-c2nc(-c3ccccc3)nc(-c3ccc(-c4ccccc4-c4ccccc4)c(-n4c5ccccc5c5c6oc7ccccc7c6ccc54)c3)n2)cc1 ; c1ccc(-c2nc(-c3ccccc3)nc(-c3ccc(-n4c5ccccc5c5c4ccc4c6ccccc6n(-c6ccccc6)c45)c(-c4ccccc4)c3)n2)cc1 ; [2H]c1c([2H])c([2H])c2c(c1[2H])c1c([2H])c([2H])c([2H])c([2H])c1n2-c1ccc(-c2nc(-c3ccccc3)nc(-c3ccccc3)n2)cc1-c1ccccc1]

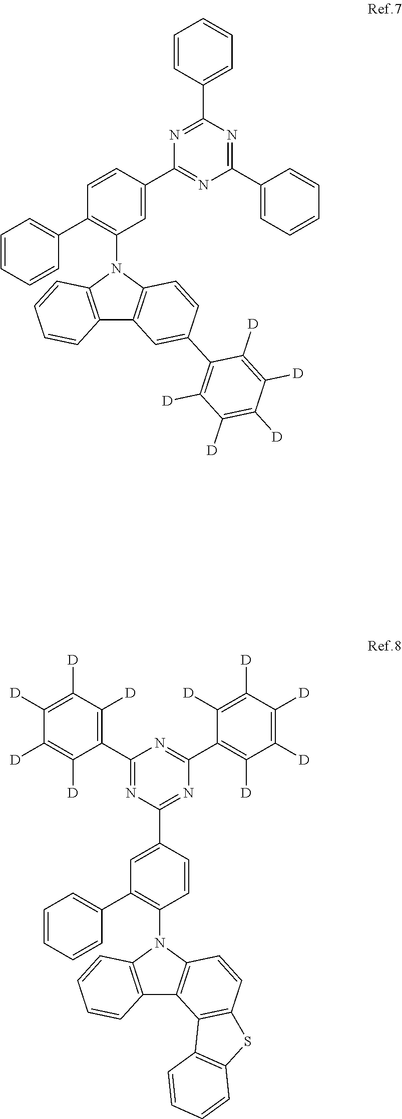 [SMILES: [2H]c1c([2H])c([2H])c(-c2ccc3c(c2)c2ccccc2n3-c2cc(-c3nc(-c4ccccc4)nc(-c4ccccc4)n3)ccc2-c2ccccc2)c([2H])c1[2H] ; [2H]c1c([2H])c([2H])c(-c2nc(-c3ccc(-n4c5ccccc5c5c6c(ccc54)sc4ccccc46)c(-c4ccccc4)c3)nc(-c3c([2H])c([2H])c([2H])c([2H])c3[2H])n2)c([2H])c1[2H]]

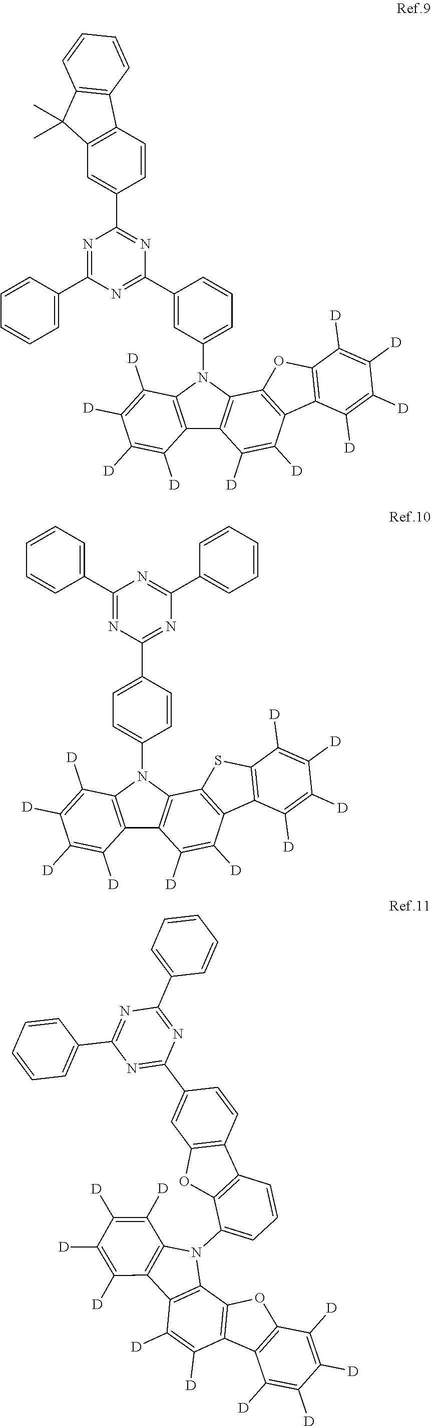

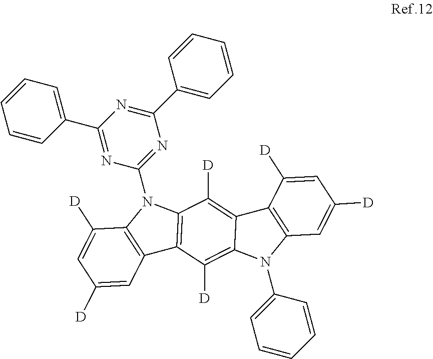 [SMILES: [2H]c1cc([2H])c2c(c1)c1c([2H])c3c(c([2H])c1n2-c1nc(-c2ccccc2)nc(-c2ccccc2)n1)c1c([2H])cc([2H])cc1n3-c1ccccc1]

### 2) Driving Voltage and Light Emitting Efficiency of Organic Electroluminescence Device

For the organic electroluminescence device manufactured as described above, electroluminescence (EL) characteristics were measured by M7000 manufactured by McScience Inc., and based on the measurement result thereof, T90 was measured by a service life measurement device (M6000) manufactured by McScience Inc., when the reference luminance was 6,000 cd/m².

The results of measuring the driving voltage, light emitting efficiency, color coordinate (CIE) and service life of the organic light emitting device manufactured according to the present invention are shown in the following Table 12.

TABLE 12

| | | | Light | | |
|---|---|---|---|---|---|
| | | Driving | emitting | | Service |
| | | voltage | efficiency | Color | life |
| | Compound | (V) | (cd/A) | coordinate | (T90) |
| Comparative | Ref. 1 | 6.49 | 20.5 | Green | 10 |
| Example 1 | | | | | |
| Comparative | Ref. 2 | 6.60 | 19.7 | Green | 25 |
| Example 2 | | | | | |
| Comparative | Ref. 3 | 6.50 | 20.5 | Green | 30 |
| Example 3 | | | | | |
| Comparative | Ref. 4 | 6.68 | 20.6 | Green | 25 |
| Example 4 | | | | | |
| Comparative | Ref. 5 | 6.74 | 18.8 | Green | 19 |
| Example 5 | | | | | |
| Comparative | Ref. 6 | 6.66 | 20.5 | Green | 25 |
| Example 6 | | | | | |
| Comparative | Ref. 7 | 6.70 | 19.5 | Green | 20 |
| Example 7 | | | | | |
| Comparative | Ref. 8 | 6.01 | 24.1 | Green | 25 |
| Example 8 | | | | | |
| Comparative | Ref. 9 | 6.11 | 20.3 | Green | 23 |
| Example 9 | | | | | |
| Comparative | Ref. 10 | 5.88 | 19.1 | Green | 19 |
| Example 10 | | | | | |
| Comparative | Ref. 11 | 5.98 | 21.3 | Green | 34 |
| Example 11 | | | | | |
| Comparative | Ref. 12 | 6.40 | 16.5 | Green | 16 |
| Example 12 | | | | | |
| Example 1 | 9 | 4.20 | 41.5 | Green | 150 |
| Example 2 | 12 | 4.12 | 45.2 | Green | 162 |
| Example 3 | 13 | 4.95 | 40.0 | Green | 80 |
| Example 4 | 17 | 4.08 | 44.6 | Green | 160 |
| Example 5 | 28 | 4.15 | 43.5 | Green | 159 |
| Example 6 | 29 | 4.00 | 45.1 | Green | 165 |
| Example 7 | 32 | 4.05 | 44.8 | Green | 170 |
| Example 8 | 38 | 4.98 | 39.2 | Green | 75 |
| Example 9 | 41 | 5.11 | 33.9 | Green | 58 |
| Example 10 | 46 | 5.28 | 34.0 | Green | 69 |
| Example 11 | 47 | 5.05 | 31.1 | Green | 50 |
| Example 12 | 59 | 5.67 | 33.2 | Green | 66 |
| Example 13 | 63 | 4.22 | 42.5 | Green | 154 |
| Example 14 | 80 | 4.11 | 43.5 | Green | 158 |
| Example 15 | 97 | 4.09 | 44.7 | Green | 172 |
| Example 16 | 106 | 4.05 | 46.6 | Green | 156 |
| Example 17 | 111 | 4.02 | 43.2 | Green | 164 |
| Example 18 | 113 | 4.94 | 35.8 | Green | 101 |
| Example 19 | 135 | 5.53 | 30.9 | Green | 40 |
| Example 20 | 136 | 5.49 | 34.1 | Green | 52 |
| Example 21 | 138 | 5.77 | 30.0 | Green | 49 |
| Example 22 | 154 | 5.70 | 34.5 | Green | 51 |
| Example 23 | 166 | 4.05 | 45.3 | Green | 156 |
| Example 24 | 178 | 4.08 | 46.6 | Green | 169 |
| Example 25 | 179 | 5.50 | 30.7 | Green | 43 |
| Example 26 | 180 | 4.01 | 45.1 | Green | 170 |
| Example 27 | 196 | 4.19 | 44.2 | Green | 163 |
| Example 28 | 197 | 4.02 | 46.8 | Green | 150 |
| Example 29 | 207 | 5.61 | 27.9 | Green | 59 |
| Example 30 | 214 | 5.70 | 33.6 | Green | 42 |
| Example 31 | 226 | 4.16 | 45.3 | Green | 149 |
| Example 32 | 247 | 4.05 | 43.8 | Green | 165 |
| Example 33 | 251 | 4.10 | 45.1 | Green | 176 |
| Example 34 | 253 | 4.49 | 40.6 | Green | 82 |
| Example 35 | 278 | 5.71 | 31.9 | Green | 76 |
| Example 36 | 298 | 4.29 | 42.2 | Green | 156 |
| Example 37 | 317 | 4.04 | 42.1 | Green | 165 |
| Example 38 | 335 | 4.00 | 43.0 | Green | 170 |
| Example 39 | 352 | 5.57 | 34.5 | Green | 50 |
| Example 40 | 376 | 4.26 | 41.5 | Green | 149 |
| Example 41 | 396 | 4.15 | 44.1 | Green | 158 |
| Example 42 | 418 | 5.61 | 31.5 | Green | 45 |
| Example 43 | 426 | 4.09 | 43.0 | Green | 159 |
| Example 44 | 440 | 4.61 | 40.1 | Green | 65 |
| Example 45 | 448 | 4.55 | 42.9 | Green | 166 |
| Example 46 | 456 | 5.50 | 33.2 | Green | 52 |
| Example 47 | 461 | 4.16 | 43.1 | Green | 152 |
| Example 48 | 479 | 4.48 | 38.5 | Green | 76 |
| Example 49 | 488 | 4.12 | 43.5 | Green | 156 |
| Example 50 | 495 | 5.70 | 33.1 | Green | 55 |
| Example 51 | 497 | 5.59 | 30.0 | Green | 60 |
| Example 52 | 520 | 4.66 | 39.5 | Green | 50 |
| Example 53 | 542 | 4.70 | 45.2 | Green | 145 |
| Example 54 | 560 | 5.66 | 31.8 | Green | 48 |

Through the results of Table 12, it can be seen that an organic light emitting device manufactured using the heterocyclic compound of the present invention as a material for a light emitting layer has better driving voltage, light emitting efficiency and service life than the Comparative Examples.

In particular, it can be seen that the compound used in Comparative Example 1 is a compound that does not include a triazine group and does not include deuterium in the carbazole fused derivative substituent, and has a high driving voltage, low light emitting efficiency, and a remarkably short service life. In contrast, the compound of the present invention includes a triazine group capable of accepting electrons to form excitons, and thus can form stable excitons due to an appropriate balance of holes and electrons. Therefore, when a device is manufactured using the compound of the present invention, the device exhibits high light emitting efficiency and service life.

In addition, the compounds used in Comparative Examples 2 to 5 are compounds whose structures include a triazine group, but does not include deuterium in the carbazole condensation derivative substituents, and it can be confirmed that the performance of the compounds deteriorates compared to the Examples. This is because in the case of compounds substituted with deuterium, which is heavier than light hydrogen, the intermolecular interactions are weakened due to a decrease in the molecular vibrational energy, resulting in a long service life result as a device with excellent durability is formed.

The compounds of Comparative Examples 6 and 7 are not carbazole fused derivatives but compounds including a carbazole group. The carbazole fused derivative of the present invention has faster hole mobility than the carbazole group due to strong hole characteristics. Therefore, the carbazole fused derivative of the present invention exhibits excellent driving characteristics, and also exhibits excellent results in terms of efficiency and service life by forming a well-balanced mobility with the triazine group having fast electron mobility.

The compound of Comparative Example 8 is a compound in which deuterium is not included in the carbazole fused derivative substituent but in the triazine group. When a triazine group is substituted with deuterium, the structural stability is better than that of carbazole, so that the stable ground energy and vibrational energy effects due to deuterium substitution are not as large as when carbazole is substituted with deuterium.

The compounds of Comparative Examples 9 and 10 are compounds in which the linker between the triazine group and the carbazole fused derivative substituent is an unsubstituted phenylene group, and in the case of the compound of the present invention having a substituted phenylene linker, the separation of HOMO and LUMO occurs, so that a more stable HOMO energy value may be formed than a compound having an unsubstituted phenylene group as a linker. This allows the formation of stable excitons and exhibits excellent efficiency and service life in device applications.

The compound of Comparative Example 11 is a compound whose linker is dibenzofuran, the compound of Comparative Example 12 is a compound which does not have a linker, and it can be seen that the driving voltage, light emitting efficiency and service life of Comparative Examples 11 and 12 in which the compound which does not satisfy the linker conditions of the present invention is used are not better than those of the examples using the heterocyclic compound of the present invention.

### Experimental Example 2

### 1) Manufacture of Organic Light Emitting Device

A glass substrate, in which ITO was thinly coated to have a thickness of 1,500 Å, was ultrasonically washed with distilled water. When the washing with distilled water was finished, the glass substrate was ultrasonically washed with a solvent such as acetone, methanol, and isopropyl alcohol, dried and then was subjected to UVO treatment for 5 minutes using UV in a UV cleaning machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

As the common layers, the hole injection layer 4,4′,4″-tris[2-naphthyl(phenyl)amino]triphenylamine (2-TNATA) and the hole transport layer N,N′-di(1-naphthyl)-N,N′-diphenyl-(1,1″-biphenyl)-4,4′-diamine (NPB) were formed on the ITO transparent electrode (positive electrode).

A light emitting layer was thermally vacuum deposited thereon as follows. Pre-mixing was performed by pre-mixing one type of heterocyclic compound of Chemical Formula 1 and one type of compound of Chemical Formula 2 or 3 described in the following Table 13 as hosts, and then the light emitting layer was deposited to have a thickness of 360 Å from one common container, and a green phosphorescent dopant was deposited by doping the host with Ir(ppy)₃ in an amount of 7% of the deposition thickness of the light emitting layer. Thereafter, BCP as a hole blocking layer was deposited to have a thickness of 60 Å, and Alq₃ as an electron transport layer was deposited to have a thickness of 200 Å thereon. Finally, lithium fluoride (LiF) was deposited to have a thickness of 10 Å on the electron transport layer to form an electron injection layer, and then an aluminum (Al) negative electrode was deposited to have a thickness of 1,200 Å on the electron injection layer to form a negative electrode, thereby manufacturing an organic electroluminescence device.

Meanwhile, all the organic compounds required for manufacturing an OLED device were subjected to vacuum sublimed purification under 10-8 to 10-6 torr for each material, and used for the manufacture of OLED.

### 2) Driving Voltage and Light Emitting Efficiency of Organic Electroluminescence Device

For the organic electroluminescence device manufactured as described above, electroluminescence (EL) characteristics were measured by M7000 manufactured by McScience Inc., and based on the measurement result thereof, T90 was measured by a service life measurement device (M6000) manufactured by McScience Inc., when the reference luminance was 6,000 cd/m².

The results of measuring the driving voltage, light emitting efficiency, color coordinate (CIE) and service life of the organic light emitting device manufactured according to the present invention are shown in the following Table 13.

TABLE 13

| | | | | | Light | | Ser- |
|---|---|---|---|---|---|---|---|
| | | | | Driving | emitting | Color | vice |
| | | | | voltage | efficiency | coor- | life |
| | Compound | | Ratio | (V) | (cd/A) | dinate | (T90) |
| Example 55 | 335 | 2-2 | 1:1 | 3.78 | 50.8 | Green | 260 |
| Example 56 | | | 1:2 | 3.81 | 53.5 | Green | 288 |
| Example 57 | | | 1:3 | 3.98 | 55.8 | Green | 305 |
| Example 58 | 352 | 2-4 | 1:1 | 5.10 | 40.1 | Green | 121 |
| Example 57 | | | 1:2 | 5.23 | 42.2 | Green | 142 |
| Example 58 | | | 1:3 | 5.49 | 43.5 | Green | 155 |
| Example 59 | 111 | 2-15 | 1:1 | 3.75 | 52.1 | Green | 275 |
| Example 60 | | | 1:2 | 3.80 | 53.3 | Green | 299 |
| Example 61 | | | 1:3 | 3.96 | 54.8 | Green | 311 |
| Example 62 | 214 | 2-31 | 1:1 | 5.05 | 39.2 | Green | 110 |
| Example 63 | | | 1:2 | 5.16 | 40.5 | Green | 135 |
| Example 64 | | | 1:3 | 5.29 | 42.2 | Green | 160 |
| Example 67 | 12 | 2-86 | 1:1 | 3.77 | 59.6 | Green | 225 |
| Example 68 | | | 1:2 | 3.80 | 60.1 | Green | 264 |
| Example 69 | | | 1:3 | 3.82 | 61.8 | Green | 290 |
| Example 70 | 46 | 2-107 | 1:1 | 4.87 | 41.0 | Green | 128 |
| Example 71 | | | 1:2 | 4.99 | 42.6 | Green | 136 |
| Example 72 | | | 1:3 | 5.01 | 45.9 | Green | 169 |
| Example 73 | 28 | 2-117 | 1:1 | 3.70 | 54.1 | Green | 299 |
| Example 74 | | | 1:2 | 3.76 | 56.8 | Green | 305 |
| Example 75 | | | 1:3 | 3.81 | 58.0 | Green | 321 |
| Example 76 | 179 | 2-6 | 1:1 | 5.21 | 39.9 | Green | 122 |
| Example 77 | | | 1:2 | 5.25 | 40.1 | Green | 150 |
| Example 78 | | | 1:3 | 5.31 | 42.2 | Green | 166 |
| Example 79 | 426 | 2-68 | 1:1 | 3.75 | 60.5 | Green | 310 |
| Example 80 | | | 1:2 | 3.78 | 62.3 | Green | 325 |
| Example 81 | | | 1:3 | 3.86 | 65.5 | Green | 350 |
| Example 82 | 46 | 2-123 | 1:1 | 4.70 | 39.8 | Green | 111 |
| Example 83 | | | 1:2 | 4.75 | 40.2 | Green | 119 |
| Example 84 | | | 1:3 | 4.80 | 43.1 | Green | 129 |
| Example 85 | 196 | 2-133 | 1:1 | 3.86 | 52.8 | Green | 308 |
| Example 86 | | | 1:2 | 3.90 | 55.2 | Green | 316 |
| Example 87 | | | 1:3 | 3.99 | 58.7 | Green | 333 |
| Example 88 | 12 | 2-135 | 1:1 | 3.60 | 62.9 | Green | 222 |
| Example 89 | | | 1:2 | 3.65 | 63.6 | Green | 260 |
| Example 90 | | | 1:3 | 3.69 | 65.0 | Green | 288 |
| Example 91 | 13 | 2-144 | 1:1 | 4.48 | 51.7 | Green | 118 |
| Example 92 | | | 1:2 | 4.52 | 53.3 | Green | 159 |
| Example 93 | | | 1:3 | 4.56 | 55.2 | Green | 197 |
| Example 94 | 59 | 2-151 | 1:1 | 5.58 | 38.8 | Green | 89 |
| Example 95 | | | 1:2 | 5.64 | 39.7 | Green | 96 |
| Example 96 | | | 1:3 | 5.65 | 40.0 | Green | 137 |
| Example 97 | 253 | 2-155 | 1:1 | 4.05 | 49.7 | Green | 106 |
| Example 98 | | | 1:2 | 4.14 | 50.3 | Green | 111 |
| Example 99 | | | 1:3 | 4.36 | 53.3 | Green | 125 |
| Example | 542 | 2-135 | 1:1 | 3.61 | 61.9 | Green | 308 |
| 100 | | | | | | | |
| Example | | | 1:2 | 3.64 | 62.6 | Green | 316 |
| 101 | | | | | | | |
| Example | | | 1:3 | 3.69 | 65.1 | Green | 333 |
| 102 | | | | | | | |

Comparing the results in Table 12 and Table 13 above, it can be confirmed that when the heterocyclic compound of Chemical Formula 1 of the present invention and the compound of Chemical Formula 2 or 3 are used together as materials for the light emitting layer, the performance of the organic light emitting device is improved, and remarkable effects, particularly in terms of light emitting efficiency and service life, are exhibited. Specifically, it can be seen that the light emitting efficiency has increased about 1.5-fold, and the service life has been extended about 2-fold.

What is claimed is:

1. A heterocyclic compound of the following Chemical Formula 1: 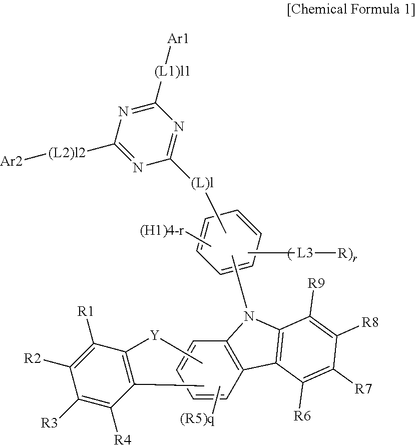
wherein, in Chemical Formula 1,
L and L1 to L3 are each independently a direct bond; a substituted or unsubstituted C6 to C60 arylene group; or a substituted or unsubstituted C2 to C60 heteroarylene group,
l, l1 and l2 are each an integer from 1 to 3, and when l, l1 and l2 are each 2 or greater, substituents in the parenthesis are the same or different,
Ar1 and Ar2 are each independently a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
R is a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
r is an integer from 1 to 4, and when r is 2 or greater, L3 and R are the same or different,
H1 is hydrogen; or deuterium, and when r is 2 or less, H1 is the same or different,
Y is O; S; C(R10)(R11) or N(R12),
R1 to R12 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group,
at least one of R1 to R9 is deuterium, and
q is 1 or 2, and when q is 2, R5 is the same or different.

2. The heterocyclic compound of claim 1, wherein Chemical Formula 1 is represented by any one of the following Chemical Formulae 1-1 to 1-3: 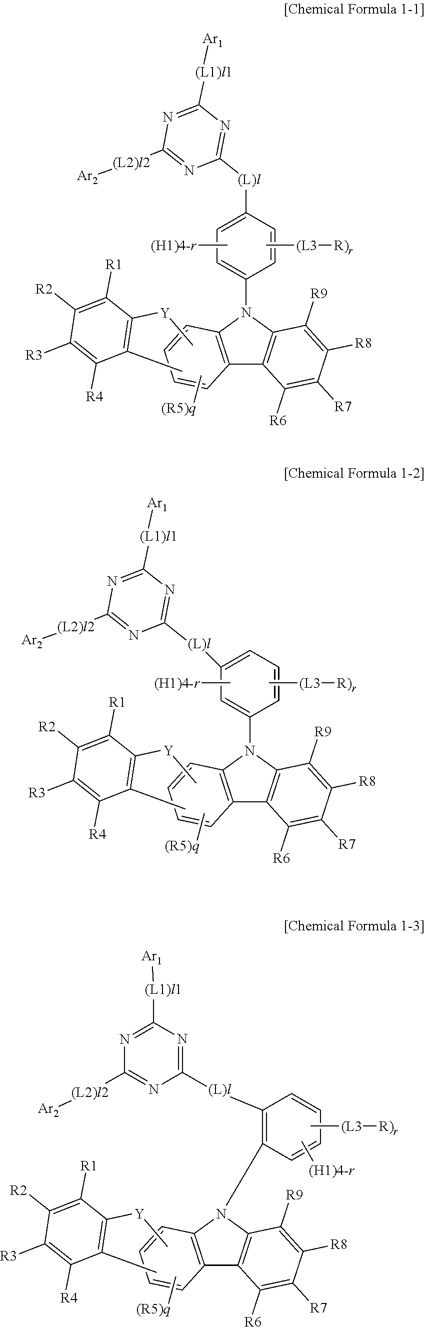
in Chemical Formulae 1-1 to 1-3,
the definition of each substituent is the same as the definition in Chemical Formula 1.

3. The heterocyclic compound of claim 1, wherein R is a substituted or unsubstituted C6 to C20 aryl group; or a substituted or unsubstituted C2 to C20 heteroaryl group.

4. The heterocyclic compound of claim 1, wherein Chemical Formula 1 is represented by [Structure A]-[Structure B]-[Structure C], Structure A is represented by the following Chemical Formula A, Structure B is represented by the following Chemical Formula B, Structure C is represented by the following Chemical Formula C, the deuterium substitution rate of Structure A is 0%, the deuterium substitution rate of Structure B is 0% to 100%, and the deuterium substitution rate of Structure C is more than 0% and 100% or less: 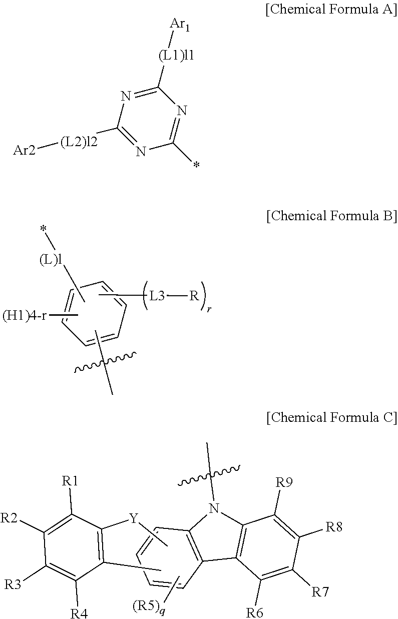
in Chemical Formulae A to C,
the definition of each substituent is the same as the definition in Chemical Formula 1, [symbol]
of Chemical Formula A is bonded to [symbol] of Chemical Formula B, and 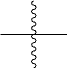
of Chemical Formula B is bonded to 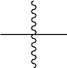
of Chemical Formula C.

5. The heterocyclic compound of claim 1, wherein the deuterium substitution rate of Chemical Formula 1 is 10% to 99%.

6. The heterocyclic compound of claim 1, wherein Chemical Formula 1 is represented by any one of the following compounds:

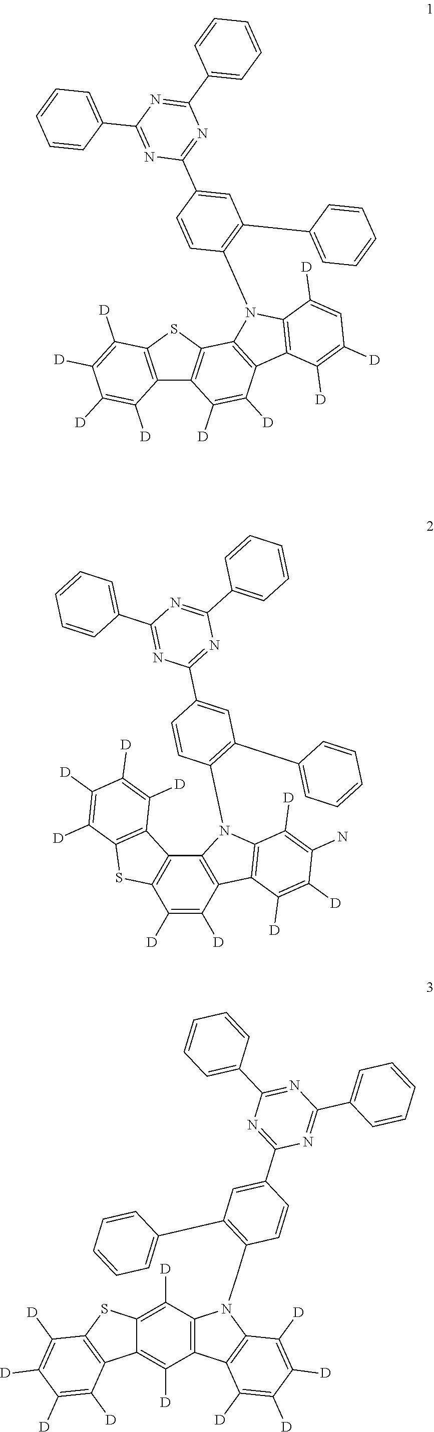 [SMILES: [2H]c1cc([2H])c2c(c1[2H])c1c([2H])c([2H])c3c(sc4c([2H])c([2H])c([2H])c([2H])c43)c1n2Cc1ccc(-c2nc(-c3ccccc3)nc(-c3ccccc3)n2)cc1Cc1ccccc1 ; [2H]c1c([2H])c([2H])c2c(sc3c([2H])c([2H])c4c5c([2H])c([2H])c(N)c([2H])c5n(Cc5ccc(-c6nc(-c7ccccc7)nc(-c7ccccc7)n6)cc5Cc5ccccc5)c4c32)c1[2H] ; [2H]c1c([2H])c([2H])c2c(sc3c([2H])c4c(c([2H])c32)c2c([2H])c([2H])c([2H])c([2H])c2n4Cc2ccc(-c3nc(-c4ccccc4)nc(-c4ccccc4)n3)cc2Cc2ccccc2)c1[2H]]

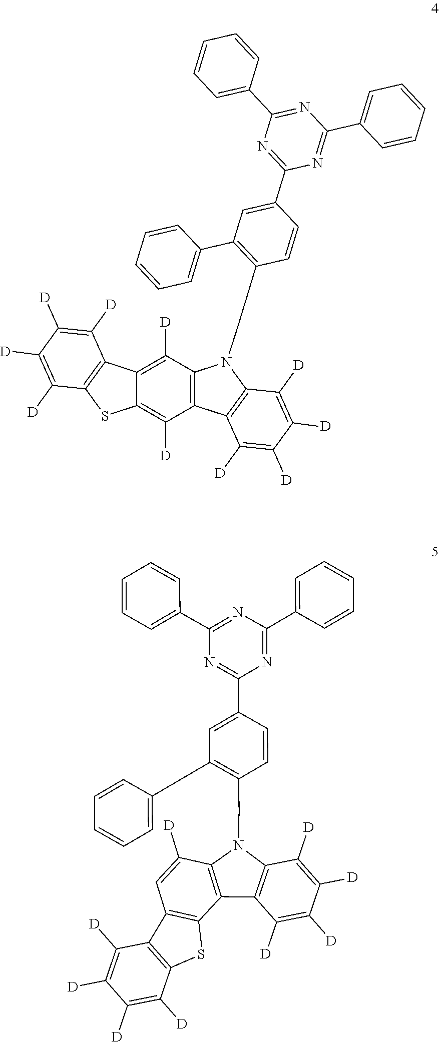 [SMILES: [2H]c1c([2H])c([2H])c2c(sc3c([2H])c4c5c([2H])c([2H])c([2H])c([2H])c5n(CCc5ccc(-c6nc(-c7ccccc7)nc(-c7ccccc7)n6)cc5-c5ccccc5)c4c([2H])c32)c1[2H] ; [2H]c1c([2H])c([2H])c2c(sc3c2cc([2H])c2c3c3c([2H])c([2H])c([2H])c([2H])c3n2Cc2ccc(-c3nc(-c4ccccc4)nc(-c4ccccc4)n3)cc2Cc2ccccc2)c1[2H]]

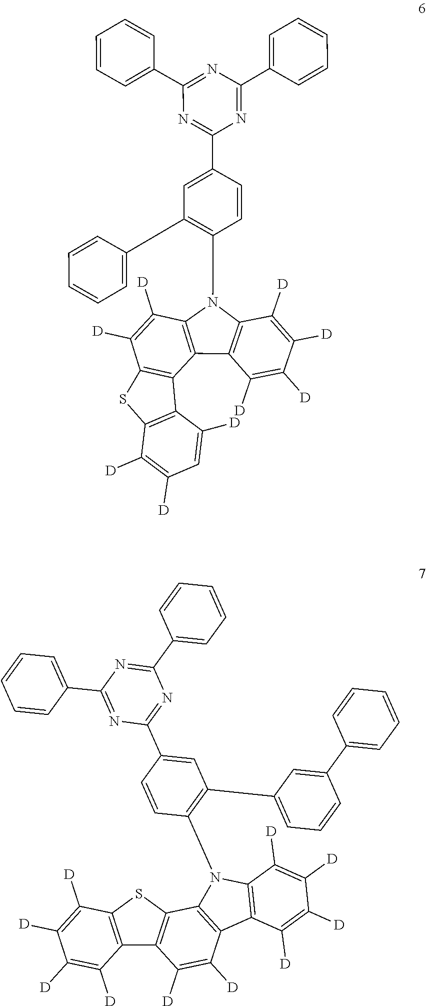 [SMILES: [2H]c1cc([2H])c2c(sc3c([2H])c([2H])c4c(c5c([2H])c([2H])c([2H])c([2H])c5n4Cc4ccc(-c5nc(-c6ccccc6)nc(-c6ccccc6)n5)cc4Cc4ccccc4)c32)c1[2H] ; [2H]c1c([2H])c([2H])c2c(sc3c2c([2H])c([2H])c2c4c([2H])c([2H])c([2H])c([2H])c4n(Cc4ccc(-c5nc(-c6ccccc6)nc(-c6ccccc6)n5)cc4Cc4cccc(-c5ccccc5)c4)c32)c1[2H]]

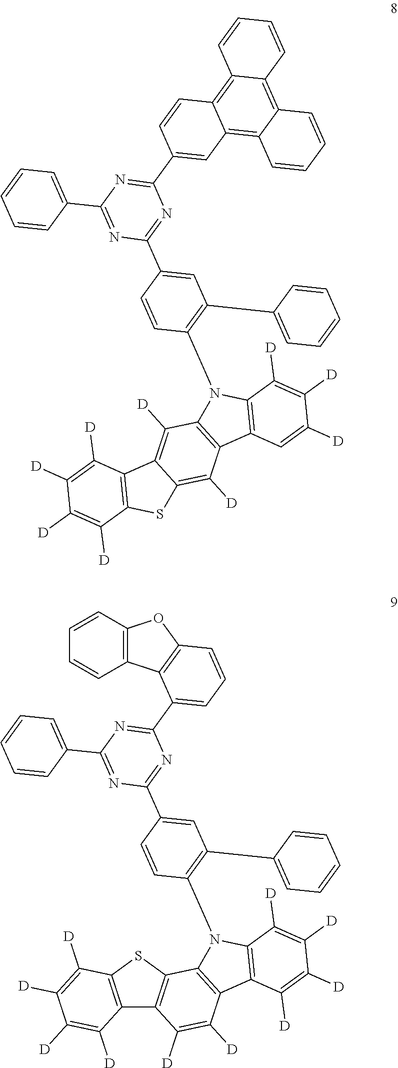 [SMILES: [2H]c1cc2c3c([2H])c4sc5c([2H])c([2H])c([2H])c([2H])c5c4c([2H])c3n(Cc3ccc(-c4nc(-c5ccccc5)nc(-c5ccc6c7ccccc7c7ccccc7c6c5)n4)cc3Cc3ccccc3)c2c([2H])c1[2H] ; [2H]c1c([2H])c([2H])c2c(sc3c2c([2H])c([2H])c2c4c([2H])c([2H])c([2H])c([2H])c4n(Cc4ccc(-c5nc(-c6ccccc6)nc(-c6cccc7oc8ccccc8c67)n5)cc4Cc4ccccc4)c32)c1[2H]]

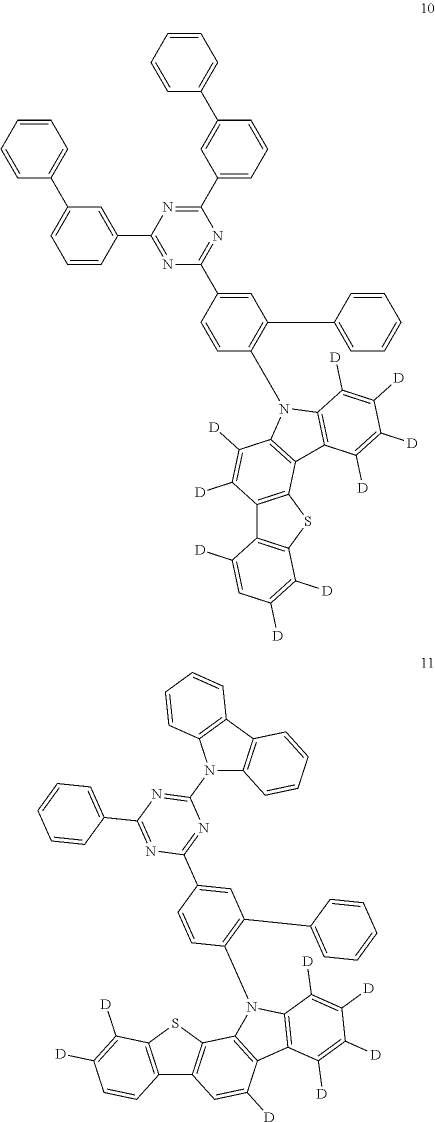 [SMILES: [2H]c1cc([2H])c2c(sc3c2c([2H])c([2H])c2c3c3c([2H])c([2H])c([2H])c([2H])c3n2Cc2ccc(-c3nc(-c4cccc(-c5ccccc5)c4)nc(-c4cccc(-c5ccccc5)c4)n3)cc2Cc2ccccc2)c1[2H] ; [2H]c1ccc2c(sc3c2cc([2H])c2c4c([2H])c([2H])c([2H])c([2H])c4n(Cc4ccc(-c5nc(-c6ccccc6)nc(-n6c7ccccc7c7ccccc76)n5)cc4Cc4ccccc4)c32)c1[2H]]

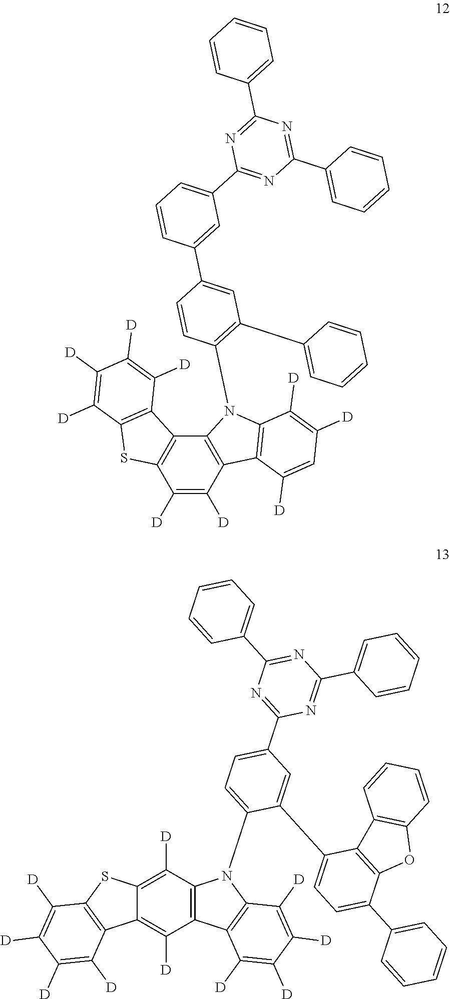 [SMILES: [2H]c1cc([2H])c2c3c([2H])c([2H])c4sc5c([2H])c([2H])c([2H])c([2H])c5c4c3n(Cc3ccc(-c4cccc(-c5nc(-c6ccccc6)nc(-c6ccccc6)n5)c4)cc3Cc3ccccc3)c2c1[2H] ; [2H]c1c([2H])c([2H])c2c(sc3c([2H])c4c(c([2H])c32)c2c([2H])c([2H])c([2H])c([2H])c2n4Cc2ccc(-c3nc(-c4ccccc4)nc(-c4ccccc4)n3)cc2Cc2ccc(-c3ccccc3)c3oc4ccccc4c23)c1[2H]]

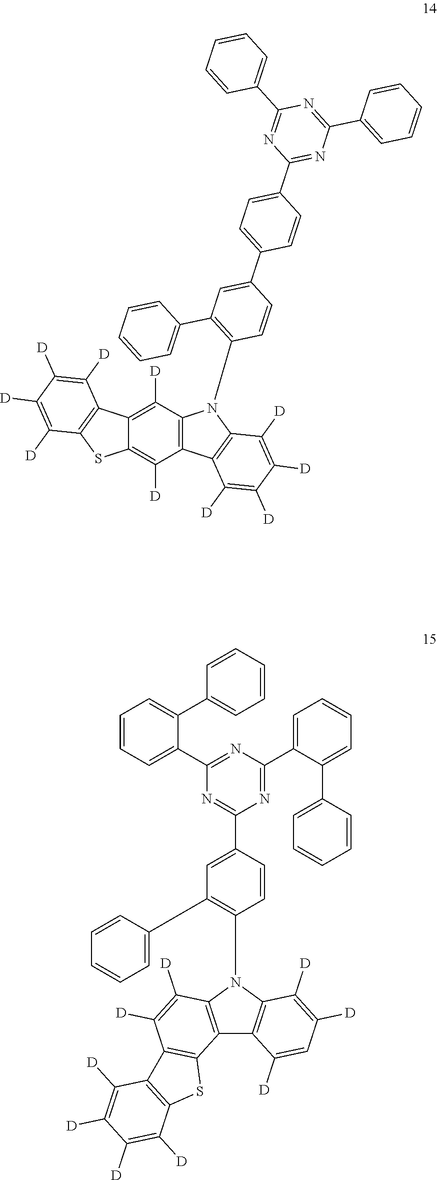 [SMILES: [2H]c1c([2H])c([2H])c2c(sc3c([2H])c4c5c([2H])c([2H])c([2H])c([2H])c5n(-c5ccc(-c6ccc(-c7nc(-c8ccccc8)nc(-c8ccccc8)n7)cc6)cc5-c5ccccc5)c4c([2H])c32)c1[2H] ; [2H]c1cc([2H])c2c3c4sc5c([2H])c([2H])c([2H])c([2H])c5c4c([2H])c([2H])c3n(Cc3ccc(-c4nc(-c5ccccc5-c5ccccc5)nc(-c5ccccc5-c5ccccc5)n4)cc3Cc3ccccc3)c2c1[2H]]

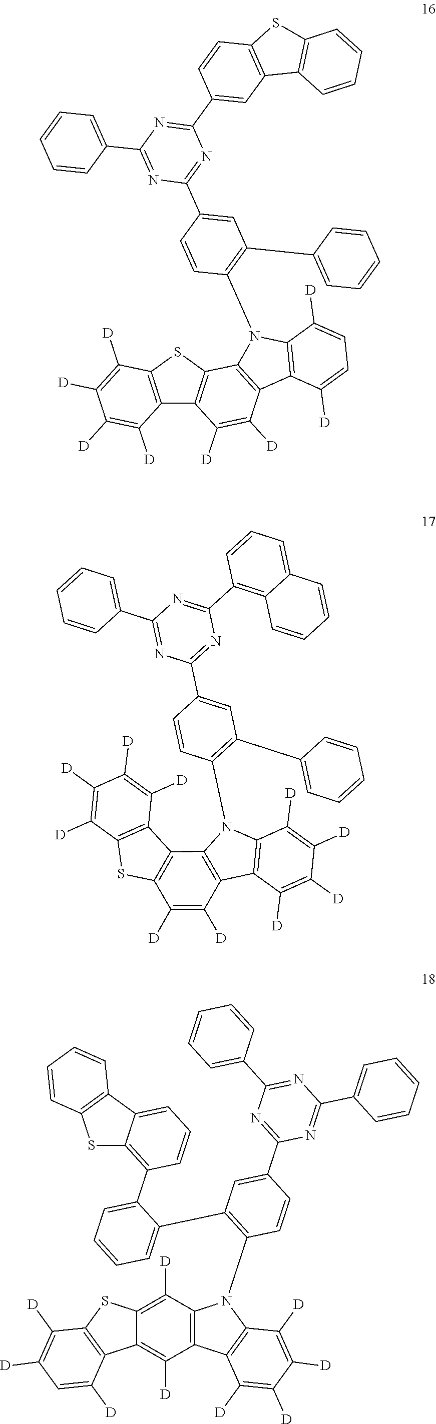 [SMILES: [2H]c1c([2H])c([2H])c2c(sc3c2c([2H])c([2H])c2c4c([2H])ccc([2H])c4n(Cc4ccc(-c5nc(-c6ccccc6)nc(-c6ccc7sc8ccccc8c7c6)n5)cc4Cc4ccccc4)c32)c1[2H] ; [2H]c1c([2H])c([2H])c2c(sc3c([2H])c([2H])c4c5c([2H])c([2H])c([2H])c([2H])c5n(Cc5ccc(-c6nc(-c7ccccc7)nc(-c7cccc8ccccc78)n6)cc5Cc5ccccc5)c4c32)c1[2H] ; [2H]c1cc([2H])c2c(sc3c([2H])c4c(c([2H])c32)c2c([2H])c([2H])c([2H])c([2H])c2n4Cc2ccc(-c3nc(-c4ccccc4)nc(-c4ccccc4)n3)cc2Cc2ccccc2-c2cccc3c2sc2ccccc23)c1[2H]]

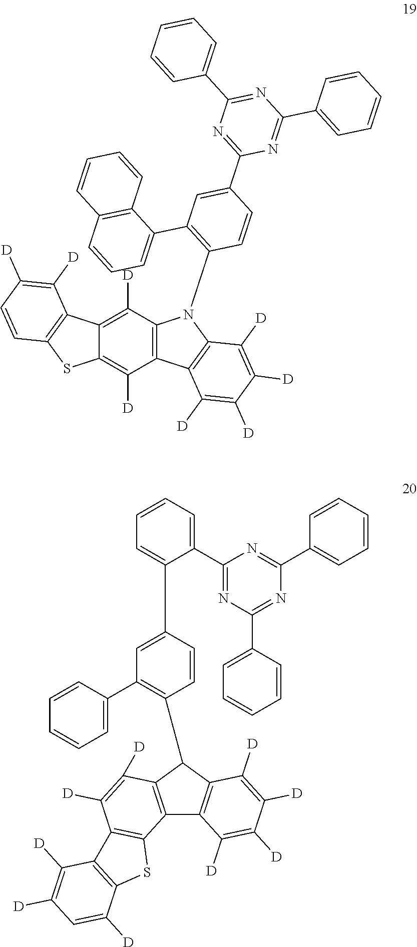 [SMILES: [2H]c1ccc2sc3c([2H])c4c5c([2H])c([2H])c([2H])c([2H])c5n(Cc5ccc(-c6nc(-c7ccccc7)nc(-c7ccccc7)n6)cc5-c5cccc6ccccc56)c4c([2H])c3c2c1[2H] ; [2H]c1cc([2H])c2sc3c4c(c([2H])c([2H])c3c2c1[2H])C(Cc1ccc(Cc2ccccc2-c2nc(-c3ccccc3)nc(-c3ccccc3)n2)cc1-c1ccccc1)c1c([2H])c([2H])c([2H])c([2H])c1-4]

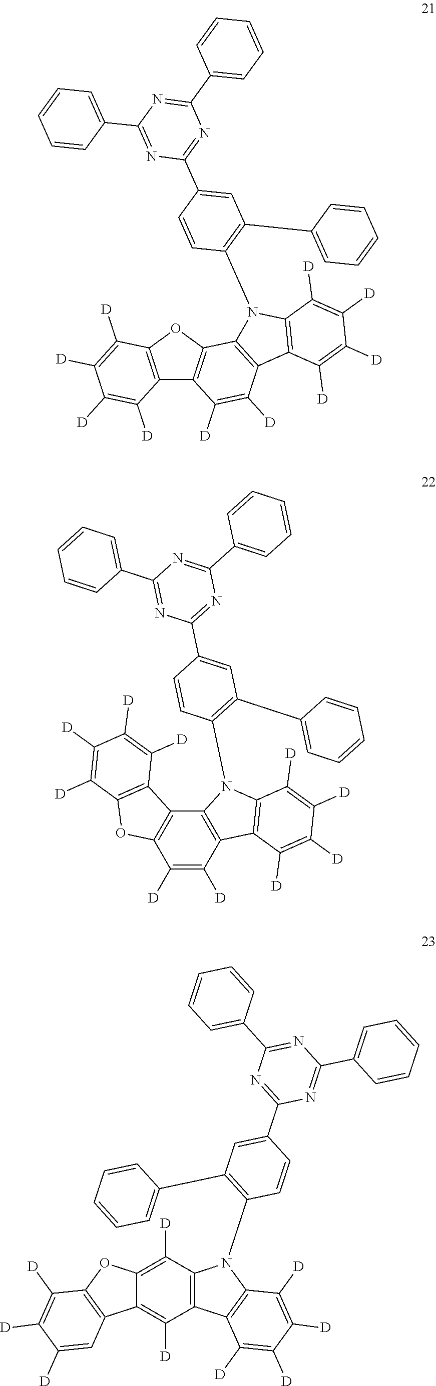 [SMILES: [2H]c1c([2H])c([2H])c2c(oc3c2c([2H])c([2H])c2c4c([2H])c([2H])c([2H])c([2H])c4n(Cc4ccc(-c5nc(-c6ccccc6)nc(-c6ccccc6)n5)cc4Cc4ccccc4)c32)c1[2H] ; [2H]c1c([2H])c([2H])c2c(oc3c([2H])c([2H])c4c5c([2H])c([2H])c([2H])c([2H])c5n(Cc5ccc(-c6nc(-c7ccccc7)nc(-c7ccccc7)n6)cc5Cc5ccccc5)c4c32)c1[2H] ; [2H]c1cc2c(oc3c([2H])c4c(c([2H])c32)c2c([2H])c([2H])c([2H])c([2H])c2n4Cc2ccc(-c3nc(-c4ccccc4)nc(-c4ccccc4)n3)cc2Cc2ccccc2)c([2H])c1[2H]]

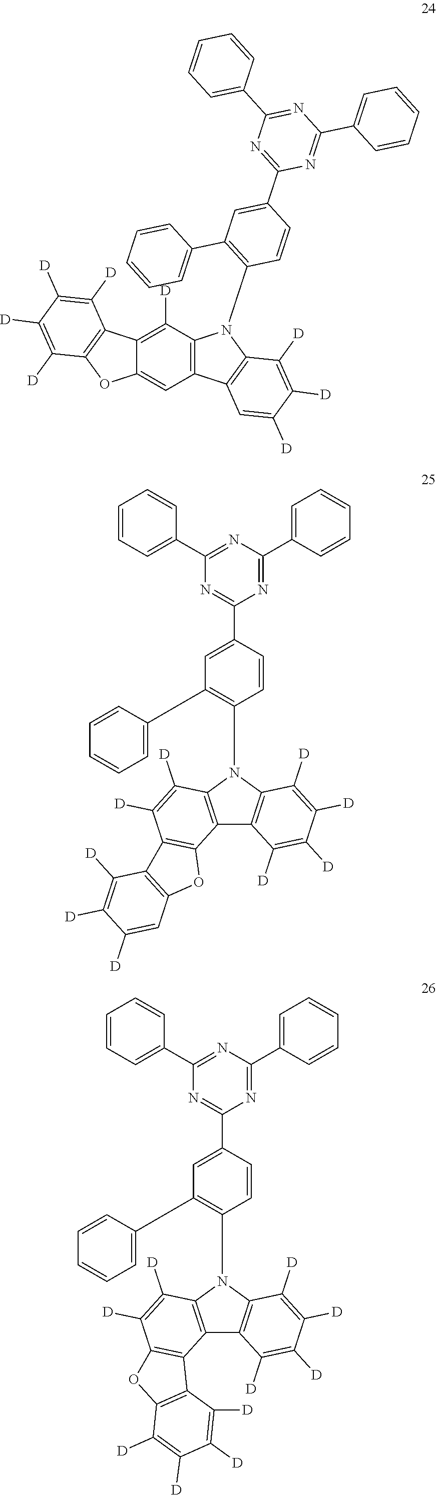

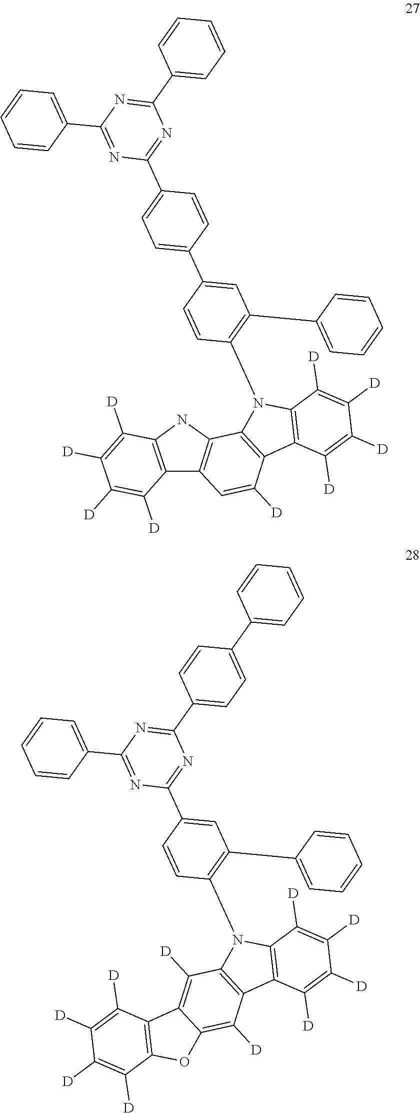 [SMILES: [2H]c1c([2H])c([2H])c2c([nH]c3c2cc([2H])c2c4c([2H])c([2H])c([2H])c([2H])c4n(Cc4ccc(-c5ccc(-c6nc(-c7ccccc7)nc(-c7ccccc7)n6)cc5)cc4Cc4ccccc4)c32)c1[2H] ; [2H]c1c([2H])c([2H])c2c(oc3c([2H])c4c5c([2H])c([2H])c([2H])c([2H])c5n(Cc5ccc(-c6nc(-c7ccccc7)nc(-c7ccc(-c8ccccc8)cc7)n6)cc5Cc5ccccc5)c4c([2H])c32)c1[2H]]

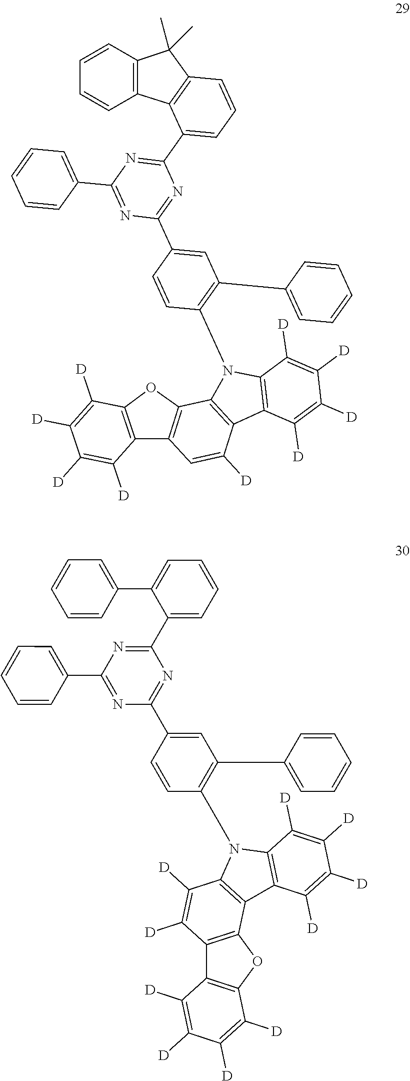 [SMILES: [2H]c1c([2H])c([2H])c2c(oc3c2cc([2H])c2c4c([2H])c([2H])c([2H])c([2H])c4n(Cc4ccc(-c5nc(-c6ccccc6)nc(-c6cccc7c6-c6ccccc6C7(C)C)n5)cc4Cc4ccccc4)c32)c1[2H] ; [2H]c1c([2H])c([2H])c2c(oc3c2c([2H])c([2H])c2c3c3c([2H])c([2H])c([2H])c([2H])c3n2Cc2ccc(-c3nc(-c4ccccc4)nc(-c4ccccc4-c4ccccc4)n3)cc2Cc2ccccc2)c1[2H]]

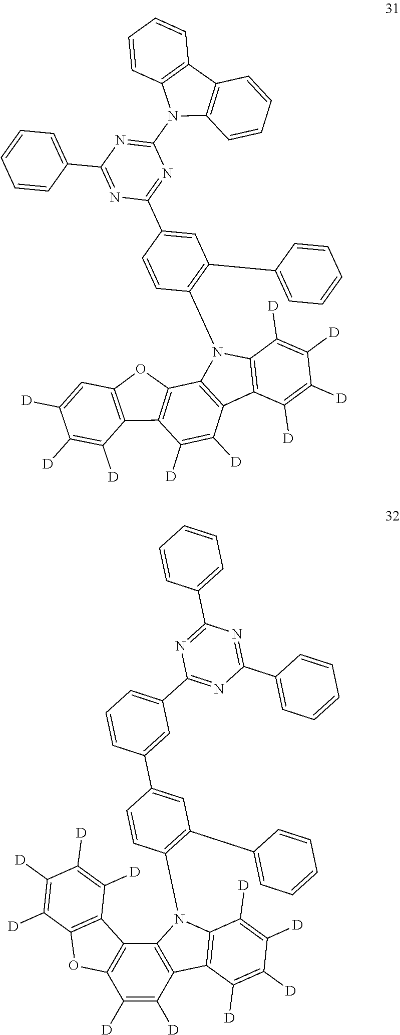 [SMILES: [2H]c1cc2oc3c(c([2H])c([2H])c4c5c([2H])c([2H])c([2H])c([2H])c5n(Cc5ccc(-c6nc(-c7ccccc7)nc(-n7c8ccccc8c8ccccc87)n6)cc5Cc5ccccc5)c34)c2c([2H])c1[2H] ; [2H]c1c([2H])c([2H])c2c(oc3c([2H])c([2H])c4c5c([2H])c([2H])c([2H])c([2H])c5n(Cc5ccc(-c6cccc(-c7nc(-c8ccccc8)nc(-c8ccccc8)n7)c6)cc5Cc5ccccc5)c4c32)c1[2H]]

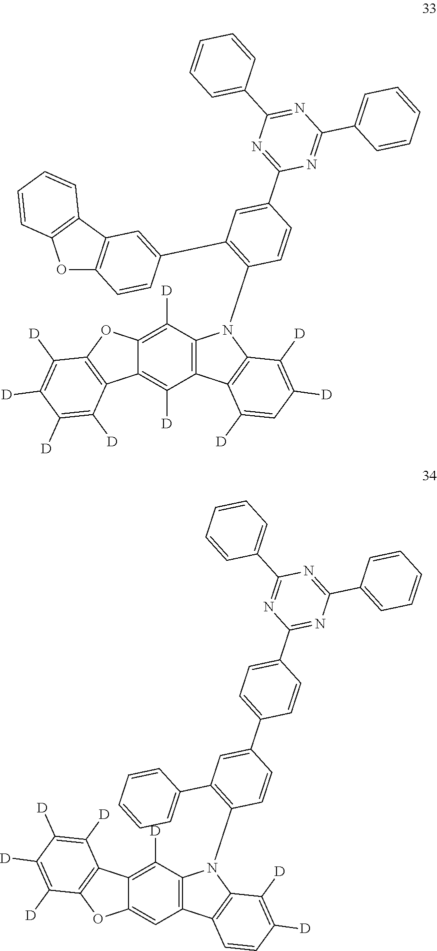 [SMILES: [2H]c1cc([2H])c2c3c([2H])c4c(oc5c([2H])c([2H])c([2H])c([2H])c54)c([2H])c3n(Cc3ccc(-c4nc(-c5ccccc5)nc(-c5ccccc5)n4)cc3Cc3ccc4oc5ccccc5c4c3)c2c1[2H] ; [2H]c1ccc2c3cc4oc5c([2H])c([2H])c([2H])c([2H])c5c4c([2H])c3n(Cc3ccc(-c4ccc(-c5nc(-c6ccccc6)nc(-c6ccccc6)n5)cc4)cc3-c3ccccc3)c2c1[2H]]

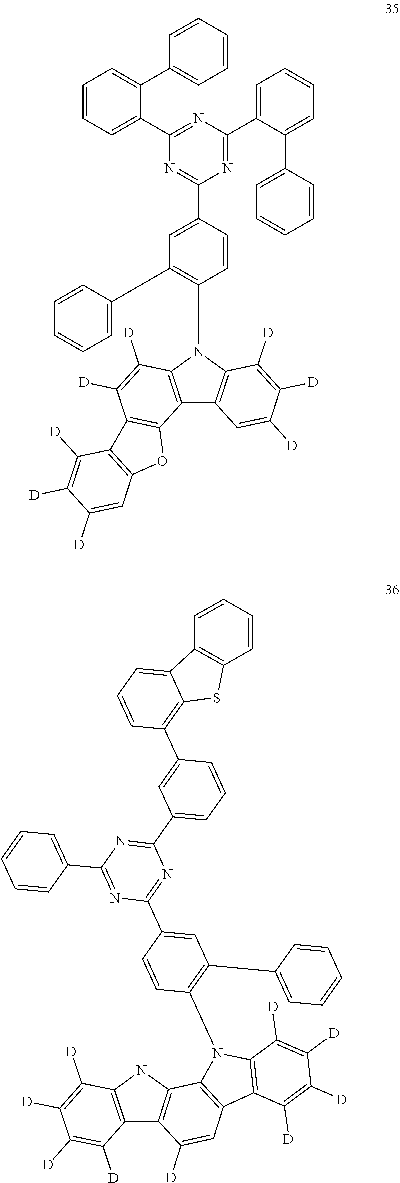 [SMILES: [2H]c1cc2oc3c(c([2H])c([2H])c4c3c3cc([2H])c([2H])c([2H])c3n4Cc3ccc(-c4nc(-c5ccccc5-c5ccccc5)nc(-c5ccccc5-c5ccccc5)n4)cc3Cc3ccccc3)c2c([2H])c1[2H] ; [2H]c1c([2H])c([2H])c2c([nH]c3c2c([2H])cc2c4c([2H])c([2H])c([2H])c([2H])c4n(Cc4ccc(-c5nc(-c6ccccc6)nc(-c6cccc(-c7cccc8c7sc7ccccc78)c6)n5)cc4Cc4ccccc4)c23)c1[2H]]

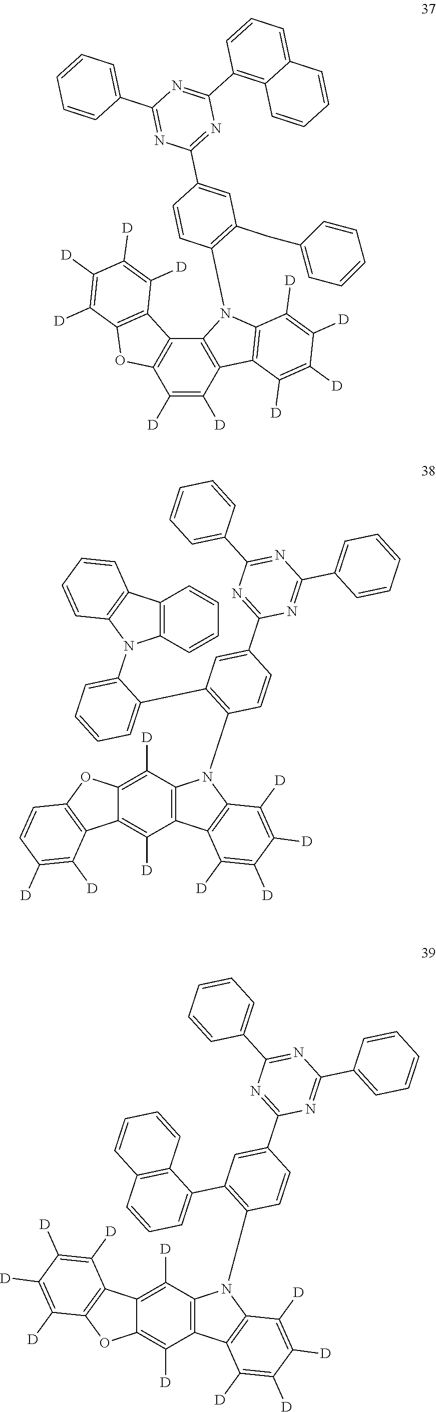 [SMILES: [2H]c1c([2H])c([2H])c2c(oc3c([2H])c([2H])c4c5c([2H])c([2H])c([2H])c([2H])c5n(Cc5ccc(-c6nc(-c7ccccc7)nc(-c7cccc8ccccc78)n6)cc5Cc5ccccc5)c4c32)c1[2H] ; [2H]c1ccc2oc3c([2H])c4c(c([2H])c3c2c1[2H])c1c([2H])c([2H])c([2H])c([2H])c1n4Cc1ccc(-c2nc(-c3ccccc3)nc(-c3ccccc3)n2)cc1Cc1ccccc1-n1c2ccccc2c2ccccc21 ; [2H]c1c([2H])c([2H])c2c(oc3c([2H])c4c5c([2H])c([2H])c([2H])c([2H])c5n(-c5ccc(-c6nc(-c7ccccc7)nc(-c7ccccc7)n6)cc5-c5cccc6ccccc56)c4c([2H])c32)c1[2H]]

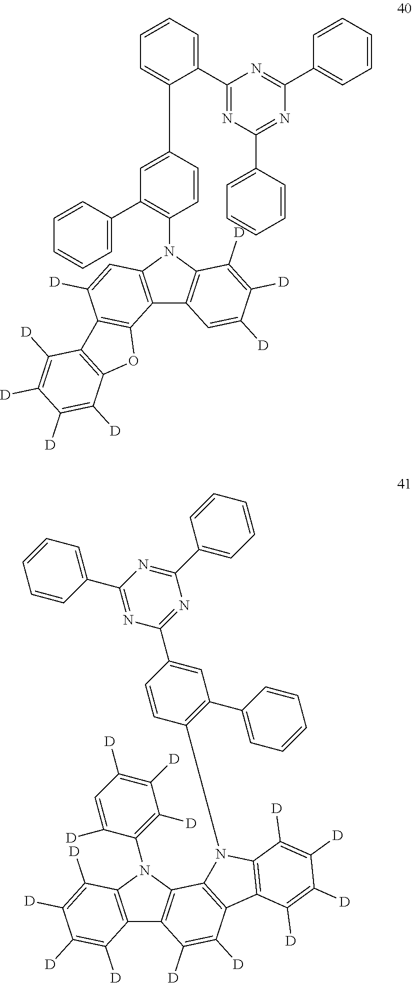 [SMILES: [2H]c1cc2c3c4oc5c([2H])c([2H])c([2H])c([2H])c5c4c([2H])cc3n(-c3ccc(Cc4ccccc4-c4nc(-c5ccccc5)nc(-c5ccccc5)n4)cc3-c3ccccc3)c2c([2H])c1[2H] ; [2H]c1cc([2H])c(-n2c3c([2H])c([2H])c([2H])c([2H])c3c3c([2H])c([2H])c4c5c([2H])c([2H])c([2H])c([2H])c5n(CCCc5ccc(-c6nc(-c7ccccc7)nc(-c7ccccc7)n6)cc5-c5ccccc5)c4c32)c([2H])c1[2H]]

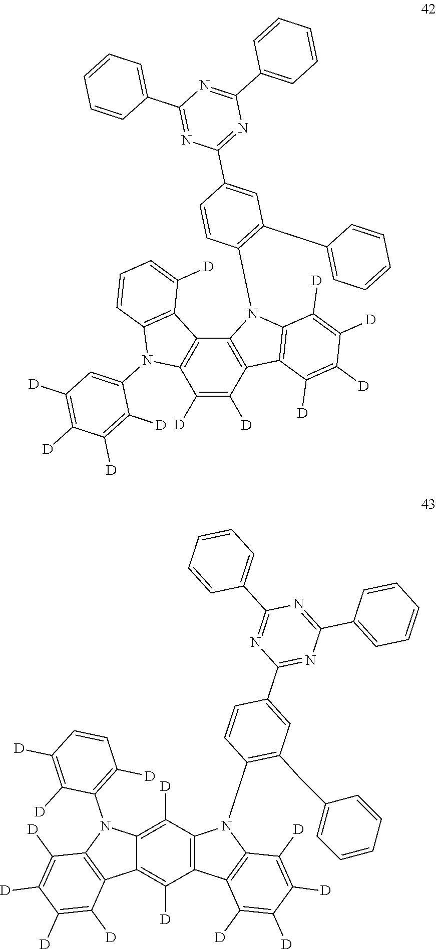 [SMILES: [2H]c1cc(-n2c3cccc([2H])c3c3c4c(c([2H])c([2H])c32)c2c([2H])c([2H])c([2H])c([2H])c2n4Cc2ccc(-c3nc(-c4ccccc4)nc(-c4ccccc4)n3)cc2Cc2ccccc2)c([2H])c([2H])c1[2H] ; [2H]c1ccc([2H])c(-n2c3c([2H])c([2H])c([2H])c([2H])c3c3c([2H])c4c5c([2H])c([2H])c([2H])c([2H])c5n(Cc5ccc(-c6nc(-c7ccccc7)nc(-c7ccccc7)n6)cc5Cc5ccccc5)c4c([2H])c32)c1[2H]]

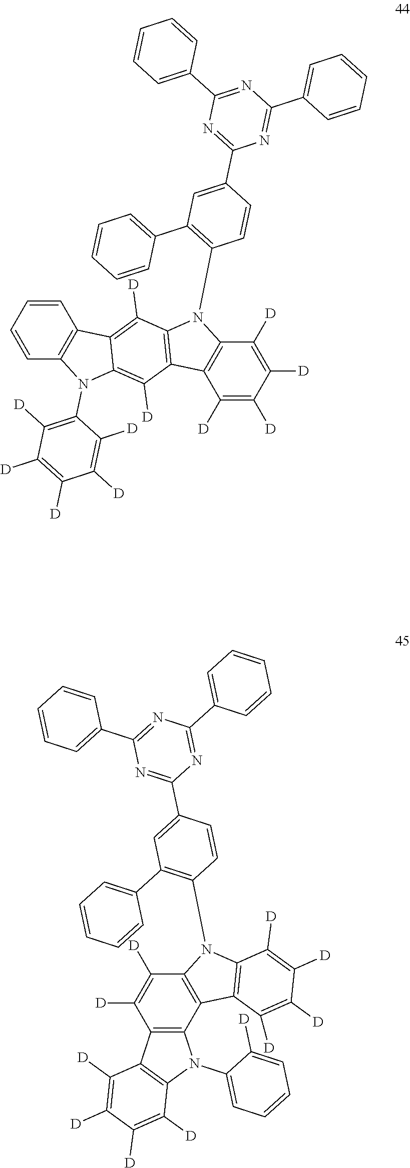 [SMILES: [2H]c1c([2H])c([2H])c(-n2c3ccccc3c3c([2H])c4c(c([2H])c32)c2c([2H])c([2H])c([2H])c([2H])c2n4-c2ccc(-c3nc(-c4ccccc4)nc(-c4ccccc4)n3)cc2-c2ccccc2)c([2H])c1[2H] ; [2H]c1ccccc1-n1c2c([2H])c([2H])c([2H])c([2H])c2c2c([2H])c([2H])c3c(c4c([2H])c([2H])c([2H])c([2H])c4n3-c3ccc(-c4nc(-c5ccccc5)nc(-c5ccccc5)n4)cc3-c3ccccc3)c21]

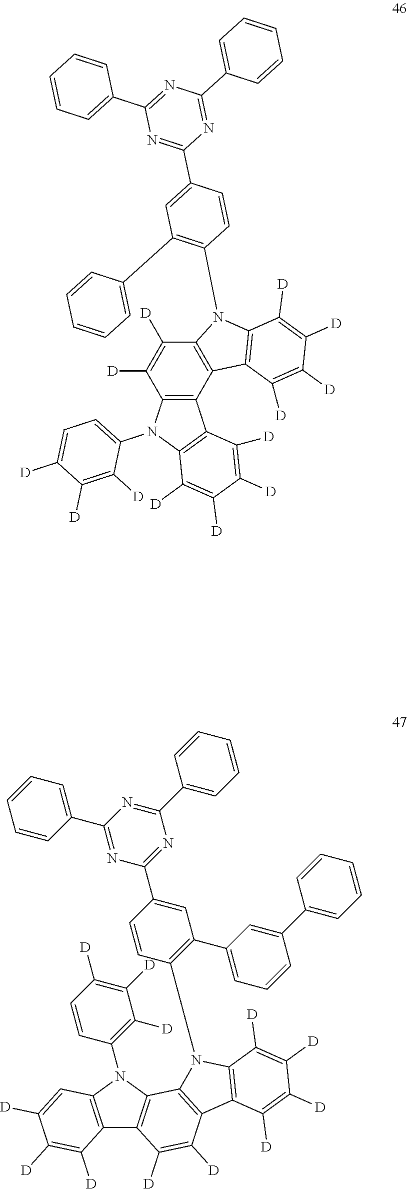 [SMILES: [2H]c1ccc(-n2c3c([2H])c([2H])c([2H])c([2H])c3c3c4c5c([2H])c([2H])c([2H])c([2H])c5n(-c5ccc(-c6nc(-c7ccccc7)nc(-c7ccccc7)n6)cc5Cc5ccccc5)c4c([2H])c([2H])c32)c([2H])c1[2H] ; [2H]c1ccc(-n2c3cc([2H])c([2H])c([2H])c3c3c([2H])c([2H])c4c5c([2H])c([2H])c([2H])c([2H])c5n(CCc5ccc(-c6nc(-c7ccccc7)nc(-c7ccccc7)n6)cc5-c5cccc(-c6ccccc6)c5)c4c32)c([2H])c1[2H]]

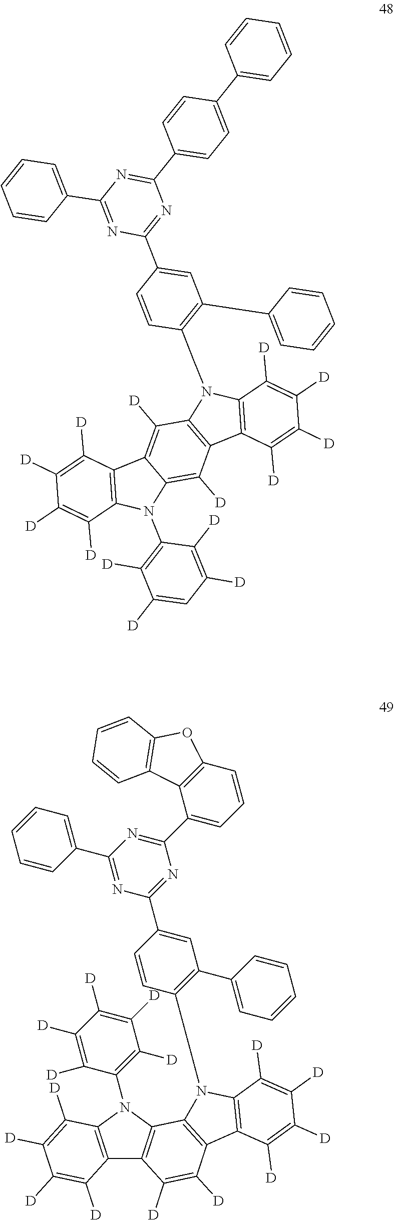 [SMILES: [2H]c1cc([2H])c([2H])c(-n2c3c([2H])c([2H])c([2H])c([2H])c3c3c([2H])c4c(c([2H])c32)c2c([2H])c([2H])c([2H])c([2H])c2n4Cc2ccc(-c3nc(-c4ccccc4)nc(-c4ccc(-c5ccccc5)cc4)n3)cc2Cc2ccccc2)c1[2H] ; [2H]c1c([2H])c([2H])c(-n2c3c([2H])c([2H])c([2H])c([2H])c3c3c([2H])c([2H])c4c5c([2H])c([2H])c([2H])c([2H])c5n(CCc5ccc(-c6nc(-c7ccccc7)nc(-c7cccc8oc9ccccc9c78)n6)cc5-c5ccccc5)c4c32)c([2H])c1[2H]]

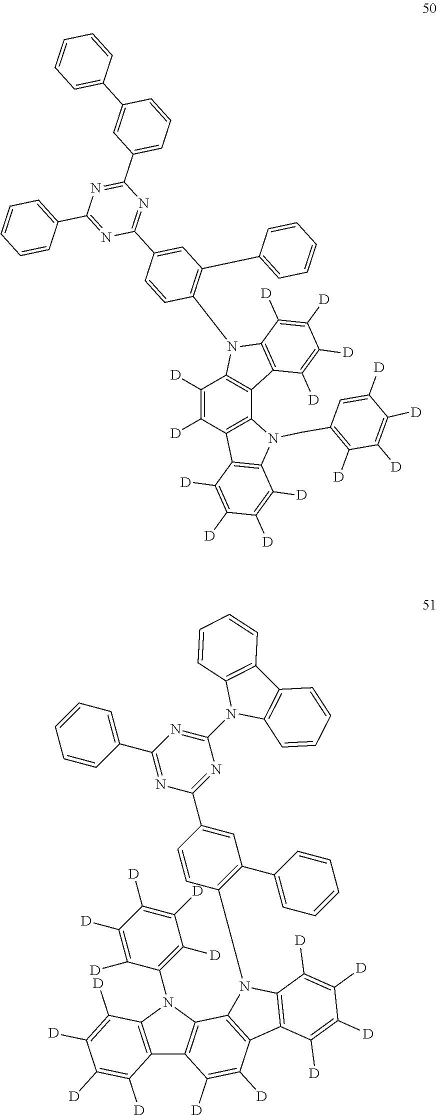 [SMILES: [2H]c1cc(Cn2c3c([2H])c([2H])c([2H])c([2H])c3c3c([2H])c([2H])c4c(c5c([2H])c([2H])c([2H])c([2H])c5n4-c4ccc(-c5nc(-c6ccccc6)nc(-c6cccc(-c7ccccc7)c6)n5)cc4Cc4ccccc4)c32)c([2H])c([2H])c1[2H] ; [2H]c1c([2H])c([2H])c(-n2c3c([2H])c([2H])c([2H])c([2H])c3c3c([2H])c([2H])c4c5c([2H])c([2H])c([2H])c([2H])c5n(CCc5ccc(-c6nc(-c7ccccc7)nc(-n7c8ccccc8c8ccccc87)n6)cc5-c5ccccc5)c4c32)c([2H])c1[2H]]

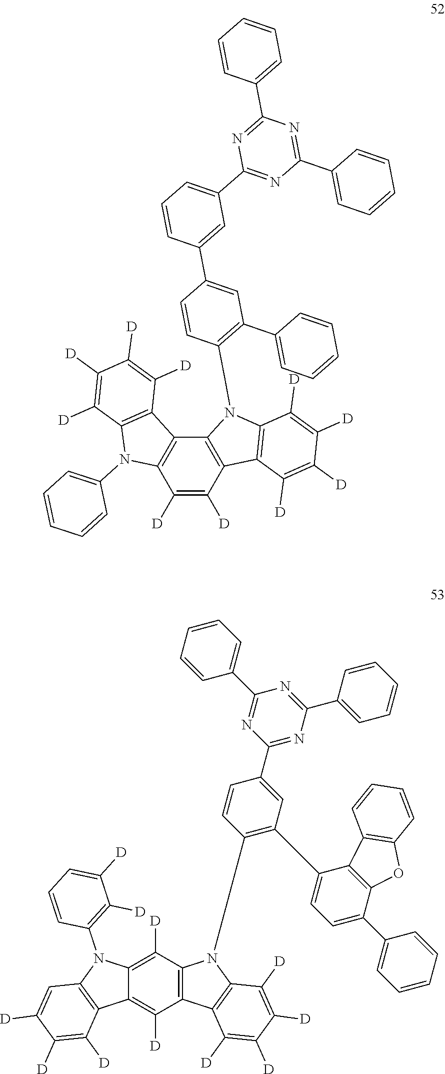 [SMILES: [2H]c1c([2H])c([2H])c2c(c1[2H])c1c([2H])c([2H])c3c(c4c([2H])c([2H])c([2H])c([2H])c4n3-c3ccccc3)c1n2Cc1ccc(-c2cccc(-c3nc(-c4ccccc4)nc(-c4ccccc4)n3)c2)cc1-c1ccccc1 ; [2H]c1cccc(-n2c3cc([2H])c([2H])c([2H])c3c3c([2H])c4c5c([2H])c([2H])c([2H])c([2H])c5n(CCCc5ccc(-c6nc(-c7ccccc7)nc(-c7ccccc7)n6)cc5Cc5ccc(-c6ccccc6)c6oc7ccccc7c56)c4c([2H])c32)c1[2H]]

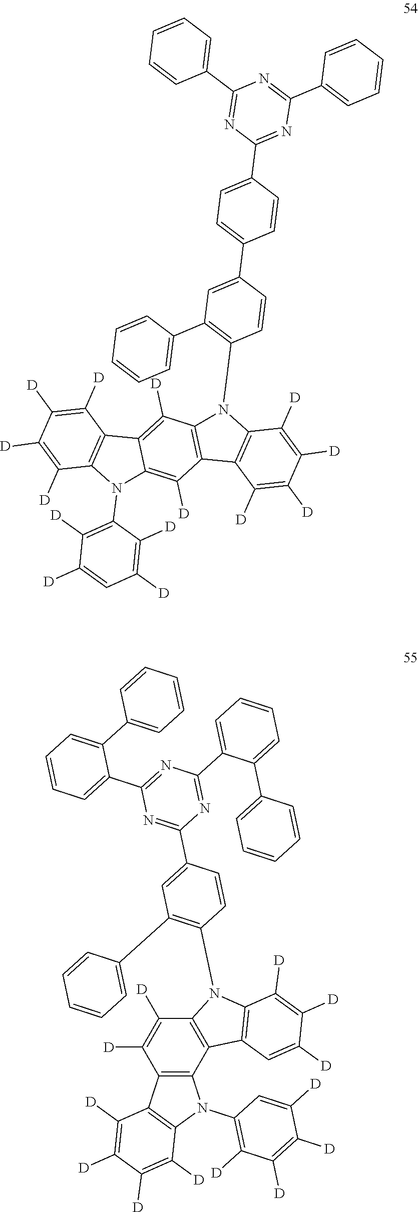 [SMILES: [2H]c1cc([2H])c([2H])c(-n2c3c([2H])c([2H])c([2H])c([2H])c3c3c([2H])c4c(c([2H])c32)c2c([2H])c([2H])c([2H])c([2H])c2n4Cc2ccc(-c3ccc(-c4nc(-c5ccccc5)nc(-c5ccccc5)n4)cc3)cc2-c2ccccc2)c1[2H] ; [2H]c1cc(-n2c3c([2H])c([2H])c([2H])c([2H])c3c3c([2H])c([2H])c4c(c5cc([2H])c([2H])c([2H])c5n4Cc4ccc(-c5nc(-c6ccccc6-c6ccccc6)nc(-c6ccccc6-c6ccccc6)n5)cc4Cc4ccccc4)c32)c([2H])c([2H])c1[2H]]

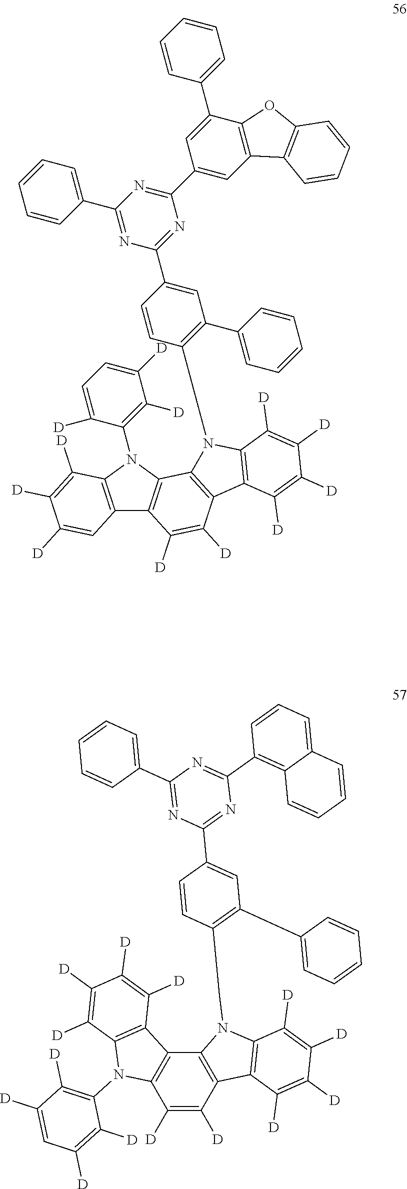 [SMILES: [2H]c1ccc([2H])c(-n2c3c([2H])c([2H])c([2H])cc3c3c([2H])c([2H])c4c5c([2H])c([2H])c([2H])c([2H])c5n(CCc5ccc(-c6nc(-c7ccccc7)nc(-c7cc(-c8ccccc8)c8oc9ccccc9c8c7)n6)cc5-c5ccccc5)c4c32)c1[2H] ; [2H]c1cc([2H])c([2H])c(-n2c3c([2H])c([2H])c([2H])c([2H])c3c3c4c(c([2H])c([2H])c32)c2c([2H])c([2H])c([2H])c([2H])c2n4CCc2ccc(-c3nc(-c4ccccc4)nc(-c4cccc5ccccc45)n3)cc2Cc2ccccc2)c1[2H]]

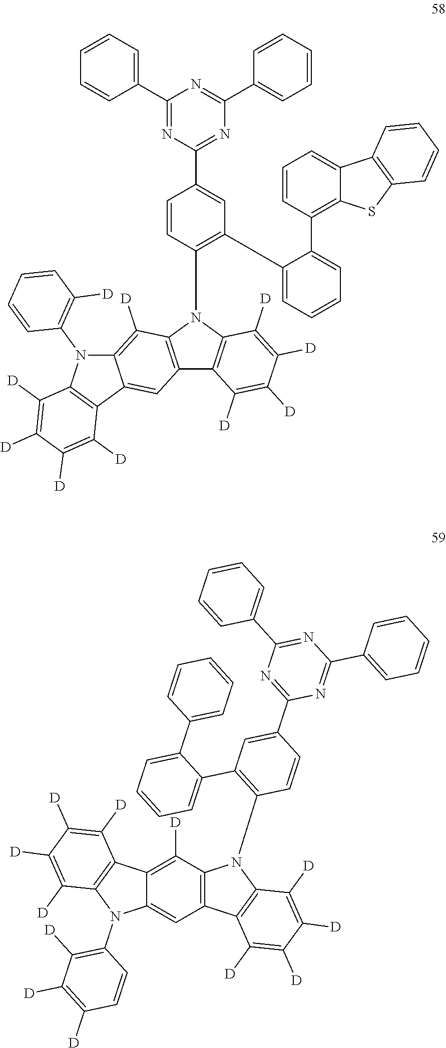 [SMILES: [2H]c1ccccc1-n1c2c([2H])c3c(cc2c2c([2H])c([2H])c([2H])c([2H])c21)c1c([2H])c([2H])c([2H])c([2H])c1n3Cc1ccc(-c2nc(-c3ccccc3)nc(-c3ccccc3)n2)cc1Cc1ccccc1-c1cccc2c1sc1ccccc12 ; [2H]c1ccc(-n2c3cc4c5c([2H])c([2H])c([2H])c([2H])c5n(-c5ccc(-c6nc(-c7ccccc7)nc(-c7ccccc7)n6)cc5-c5ccccc5-c5ccccc5)c4c([2H])c3c3c([2H])c([2H])c([2H])c([2H])c32)c([2H])c1[2H]]

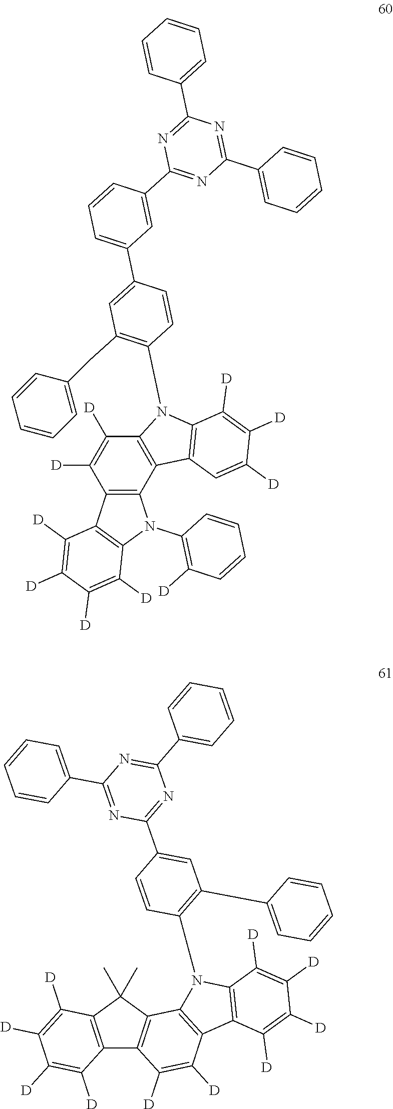 [SMILES: [2H]c1ccccc1-n1c2c([2H])c([2H])c([2H])c([2H])c2c2c([2H])c([2H])c3c(c4cc([2H])c([2H])c([2H])c4n3Cc3ccc(-c4cccc(-c5nc(-c6ccccc6)nc(-c6ccccc6)n5)c4)cc3Cc3ccccc3)c21 ; [2H]c1c([2H])c([2H])c2c(c1[2H])-c1c([2H])c([2H])c3c4c([2H])c([2H])c([2H])c([2H])c4n(Cc4ccc(-c5nc(-c6ccccc6)nc(-c6ccccc6)n5)cc4Cc4ccccc4)c3c1C2(C)C]

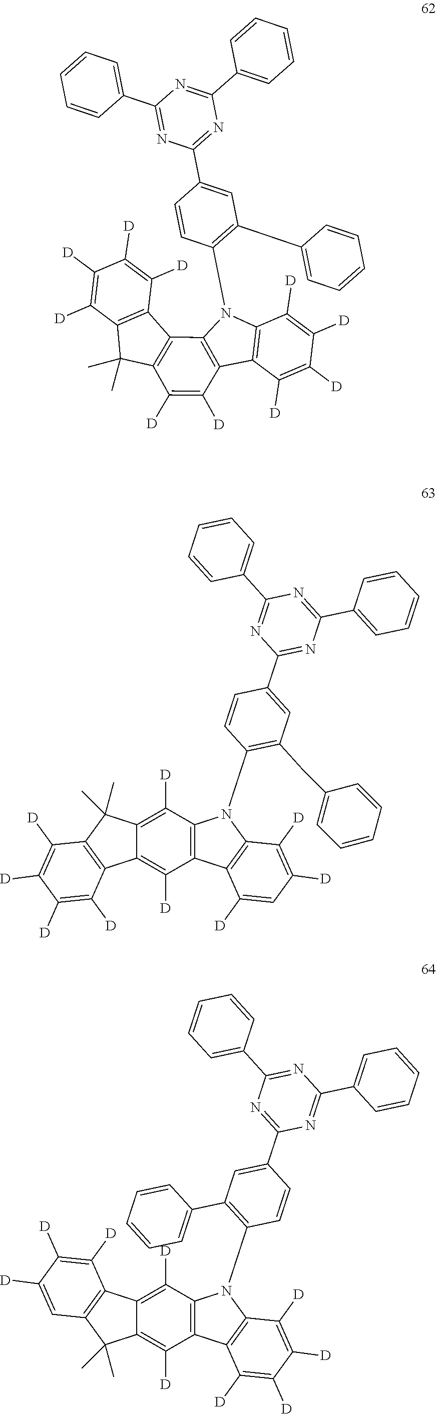 [SMILES: [2H]c1c([2H])c([2H])c2c(c1[2H])-c1c(c([2H])c([2H])c3c4c([2H])c([2H])c([2H])c([2H])c4n(Cc4ccc(-c5nc(-c6ccccc6)nc(-c6ccccc6)n5)cc4Cc4ccccc4)c13)C2(C)C ; [2H]c1cc([2H])c2c3c([2H])c4c(c([2H])c3n(Cc3ccc(-c5nc(-c6ccccc6)nc(-c6ccccc6)n5)cc3Cc3ccccc3)c2c1[2H])C(C)(C)c1c([2H])c([2H])c([2H])c([2H])c1-4 ; [2H]c1cc2c(c([2H])c1[2H])-c1c(c([2H])c3c4c([2H])c([2H])c([2H])c([2H])c4n(Cc4ccc(-c5nc(-c6ccccc6)nc(-c6ccccc6)n5)cc4-c4ccccc4)c3c1[2H])C2(C)C]

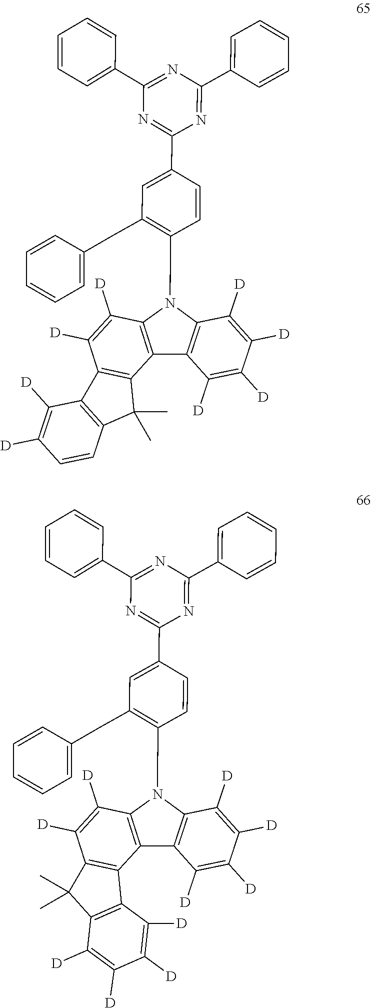 [SMILES: [2H]c1ccc2c(c1[2H])-c1c([2H])c([2H])c3c(c1C2(C)C)c1c([2H])c([2H])c([2H])c([2H])c1n3Cc1ccc(-c2nc(-c3ccccc3)nc(-c3ccccc3)n2)cc1Cc1ccccc1 ; [2H]c1c([2H])c([2H])c2c(c1[2H])-c1c(c([2H])c([2H])c3c1c1c([2H])c([2H])c([2H])c([2H])c1n3Cc1ccc(-c3nc(-c4ccccc4)nc(-c4ccccc4)n3)cc1Cc1ccccc1)C2(C)C]

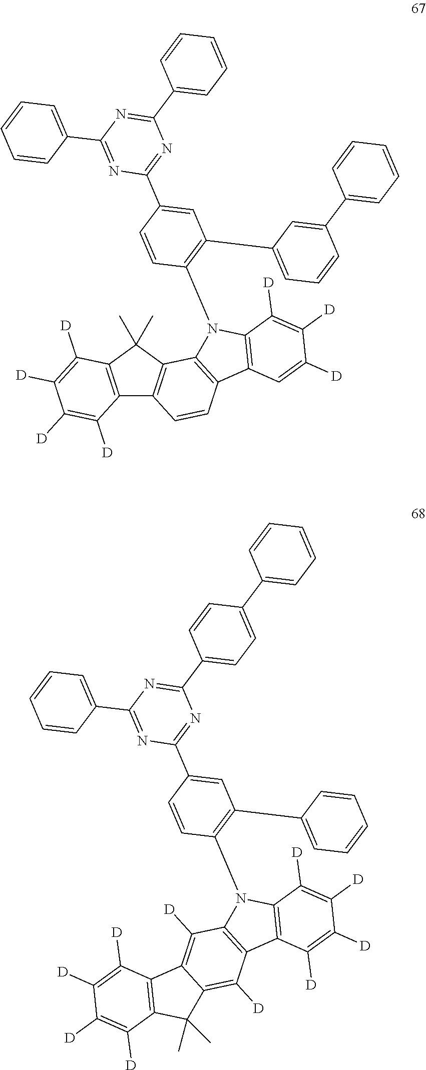 [SMILES: [2H]c1cc2c3ccc4c(c3n(Cc3ccc(-c5nc(-c6ccccc6)nc(-c6ccccc6)n5)cc3Cc3cccc(-c5ccccc5)c3)c2c([2H])c1[2H])C(C)(C)c1c([2H])c([2H])c([2H])c([2H])c1-4 ; [2H]c1c([2H])c([2H])c2c(c1[2H])-c1c(c([2H])c3c4c([2H])c([2H])c([2H])c([2H])c4n(Cc4ccc(-c5nc(-c6ccccc6)nc(-c6ccc(-c7ccccc7)cc6)n5)cc4Cc4ccccc4)c3c1[2H])C2(C)C]

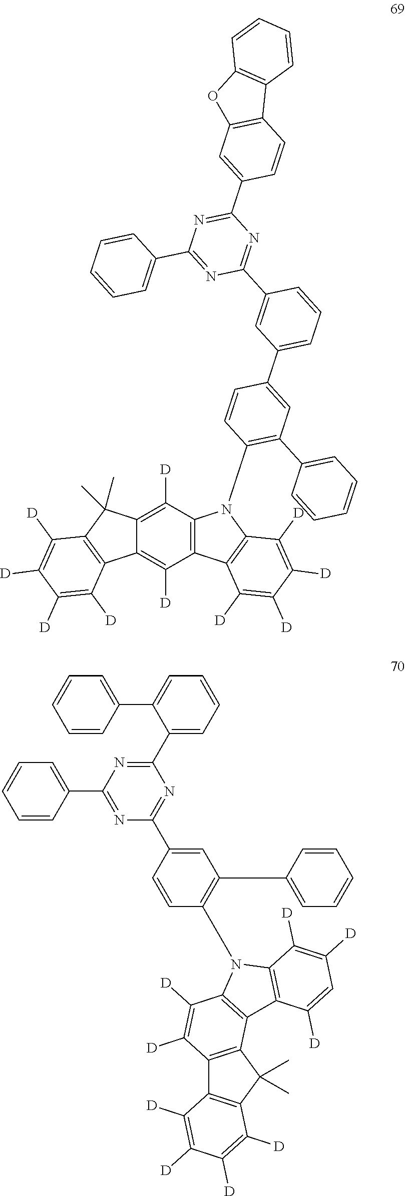 [SMILES: [2H]c1c([2H])c([2H])c2c(c1[2H])-c1c(c([2H])c3c(c1[2H])c1c([2H])c([2H])c([2H])c([2H])c1n3Cc1ccc(-c3cccc(-c4nc(-c5ccccc5)nc(-c5ccc6c(c5)oc5ccccc56)n4)c3)cc1-c1ccccc1)C2(C)C ; [2H]c1cc([2H])c2c3c4c(c([2H])c([2H])c3n(Cc3ccc(-c5nc(-c6ccccc6)nc(-c6ccccc6-c6ccccc6)n5)cc3Cc3ccccc3)c2c1[2H])-c1c([2H])c([2H])c([2H])c([2H])c1C4(C)C]

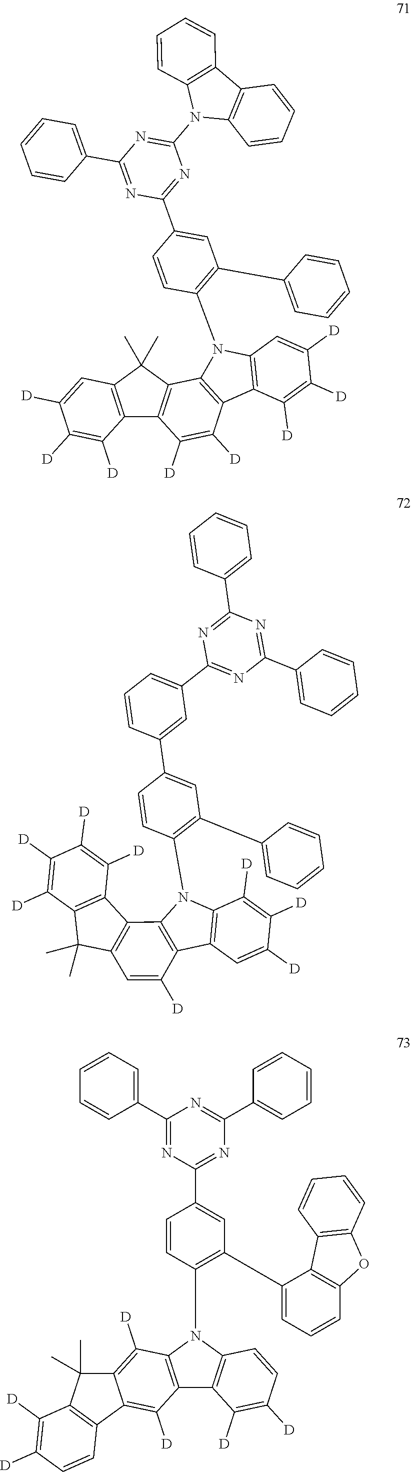 [SMILES: [2H]c1cc2c(c([2H])c1[2H])-c1c([2H])c([2H])c3c4c([2H])c([2H])c([2H])cc4n(-c4ccc(-c5nc(-c6ccccc6)nc(-n6c7ccccc7c7ccccc76)n5)cc4Cc4ccccc4)c3c1C2(C)C ; [2H]c1cc2c3c([2H])cc4c(c3n(-c3ccc(-c5cccc(-c6nc(-c7ccccc7)nc(-c7ccccc7)n6)c5)cc3Cc3ccccc3)c2c([2H])c1[2H])-c1c([2H])c([2H])c([2H])c([2H])c1C4(C)C ; [2H]c1ccc2c(c1[2H])C(C)(C)c1c-2c([2H])c2c3c([2H])c([2H])ccc3n(Cc3ccc(-c4nc(-c5ccccc5)nc(-c5ccccc5)n4)cc3Cc3cccc4oc5ccccc5c34)c2c1[2H]]

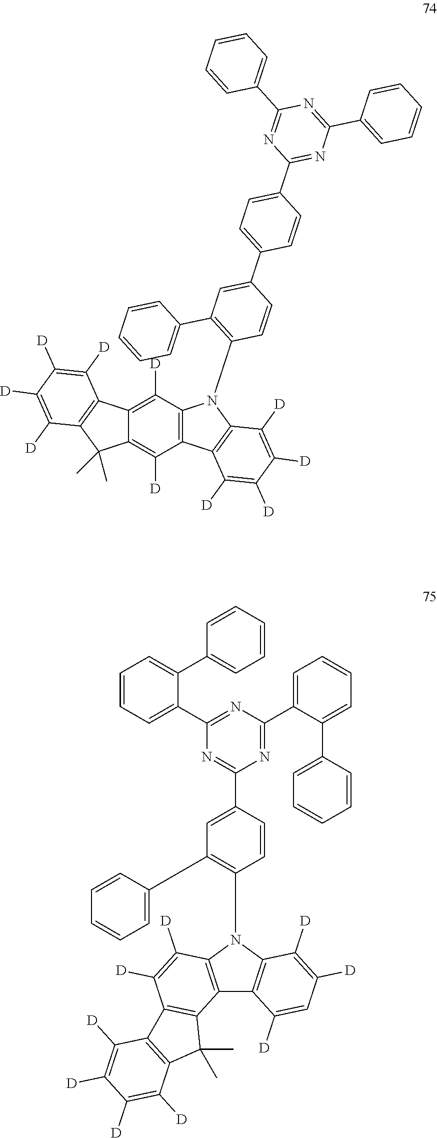 [SMILES: [2H]c1c([2H])c([2H])c2c(c1[2H])-c1c(c([2H])c3c4c([2H])c([2H])c([2H])c([2H])c4n(Cc4ccc(-c5ccc(-c6nc(-c7ccccc7)nc(-c7ccccc7)n6)cc5)cc4-c4ccccc4)c3c1[2H])C2(C)C ; [2H]c1cc([2H])c2c3c4c(c([2H])c([2H])c3n(Cc3ccc(-c5nc(-c6ccccc6-c6ccccc6)nc(-c6ccccc6-c6ccccc6)n5)cc3Cc3ccccc3)c2c1[2H])-c1c([2H])c([2H])c([2H])c([2H])c1C4(C)C]

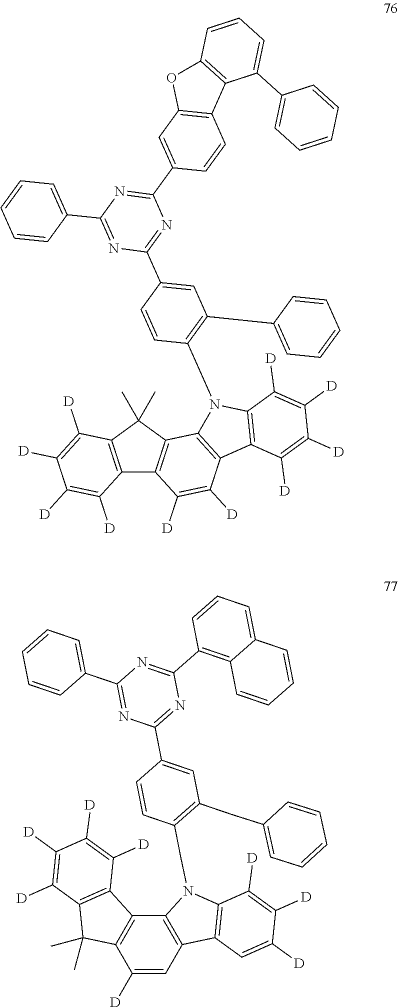 [SMILES: [2H]c1c([2H])c([2H])c2c(c1[2H])-c1c([2H])c([2H])c3c4c([2H])c([2H])c([2H])c([2H])c4n(Cc4ccc(-c5nc(-c6ccccc6)nc(-c6ccc7c(c6)oc6cccc(-c8ccccc8)c67)n5)cc4Cc4ccccc4)c3c1C2(C)C ; [2H]c1cc2c3cc([2H])c4c(c3n(Cc3ccc(-c5nc(-c6ccccc6)nc(-c6cccc7ccccc67)n5)cc3Cc3ccccc3)c2c([2H])c1[2H])-c1c([2H])c([2H])c([2H])c([2H])c1C4(C)C]

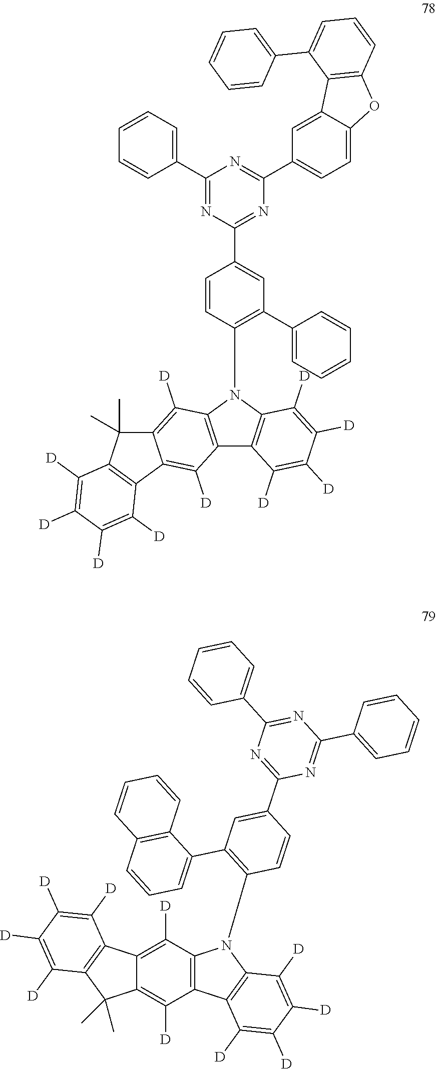 [SMILES: [2H]c1c([2H])c([2H])c2c(c1[2H])-c1c(c([2H])c3c(c1[2H])c1c([2H])c([2H])c([2H])c([2H])c1n3Cc1ccc(-c3nc(-c4ccccc4)nc(-c4ccc5oc6cccc(-c7ccccc7)c6c5c4)n3)cc1-c1ccccc1)C2(C)C ; [2H]c1c([2H])c([2H])c2c(c1[2H])-c1c(c([2H])c3c4c([2H])c([2H])c([2H])c([2H])c4n(-c4ccc(-c5nc(-c6ccccc6)nc(-c6ccccc6)n5)cc4-c4cccc5ccccc45)c3c1[2H])C2(C)C]

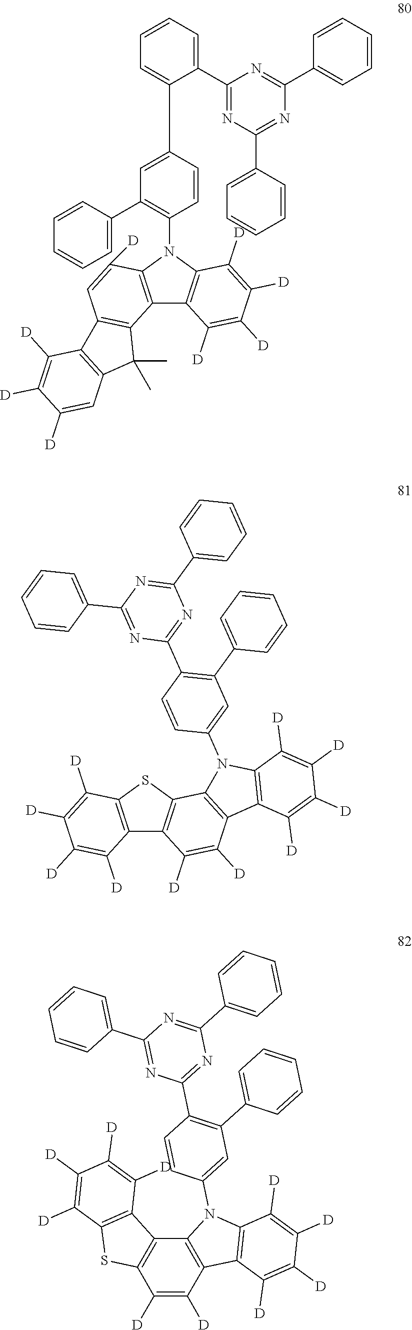 [SMILES: [2H]c1cc2c(c([2H])c1[2H])-c1cc([2H])c3c(c1C2(C)C)c1c([2H])c([2H])c([2H])c([2H])c1n3-c1ccc(Cc2ccccc2-c2nc(-c3ccccc3)nc(-c3ccccc3)n2)cc1-c1ccccc1 ; [2H]c1c([2H])c([2H])c2c(sc3c2c([2H])c([2H])c2c4c([2H])c([2H])c([2H])c([2H])c4n(-c4ccc(-c5nc(-c6ccccc6)nc(-c6ccccc6)n5)c(-c5ccccc5)c4)c32)c1[2H] ; [2H]c1c([2H])c([2H])c2c(sc3c([2H])c([2H])c4c5c([2H])c([2H])c([2H])c([2H])c5n(-c5ccc(-c6nc(-c7ccccc7)nc(-c7ccccc7)n6)c(-c6ccccc6)c5)c4c32)c1[2H]]

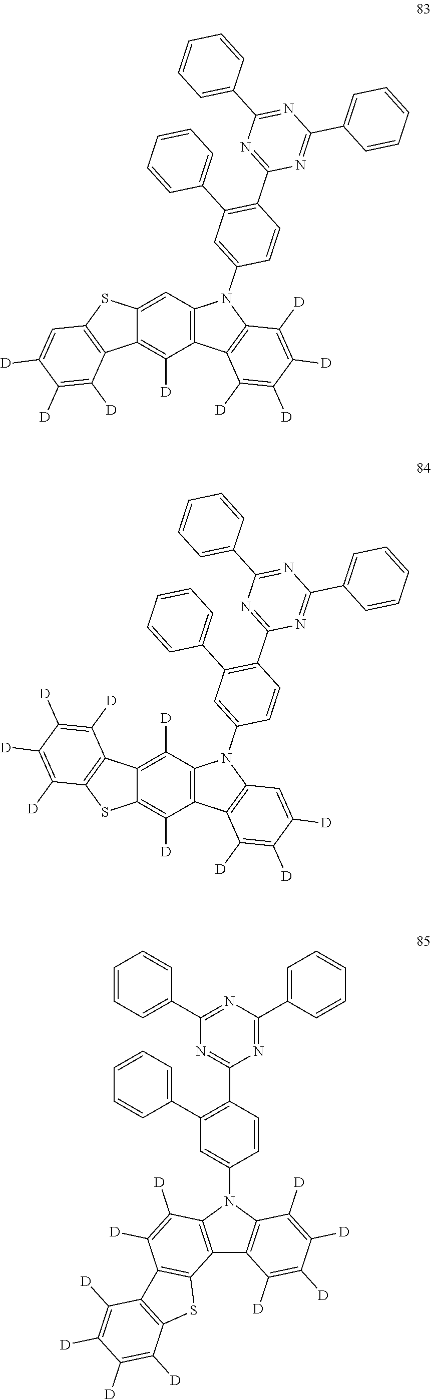 [SMILES: [2H]c1cc2sc3cc4c(c([2H])c3c2c([2H])c1[2H])c1c([2H])c([2H])c([2H])c([2H])c1n4-c1ccc(-c2nc(-c3ccccc3)nc(-c3ccccc3)n2)c(-c2ccccc2)c1 ; [2H]c1cc2c(c([2H])c1[2H])c1c([2H])c3sc4c([2H])c([2H])c([2H])c([2H])c4c3c([2H])c1n2-c1ccc(-c2nc(-c3ccccc3)nc(-c3ccccc3)n2)c(-c2ccccc2)c1 ; [2H]c1c([2H])c([2H])c2c(sc3c2c([2H])c([2H])c2c3c3c([2H])c([2H])c([2H])c([2H])c3n2-c2ccc(-c3nc(-c4ccccc4)nc(-c4ccccc4)n3)c(-c3ccccc3)c2)c1[2H]]

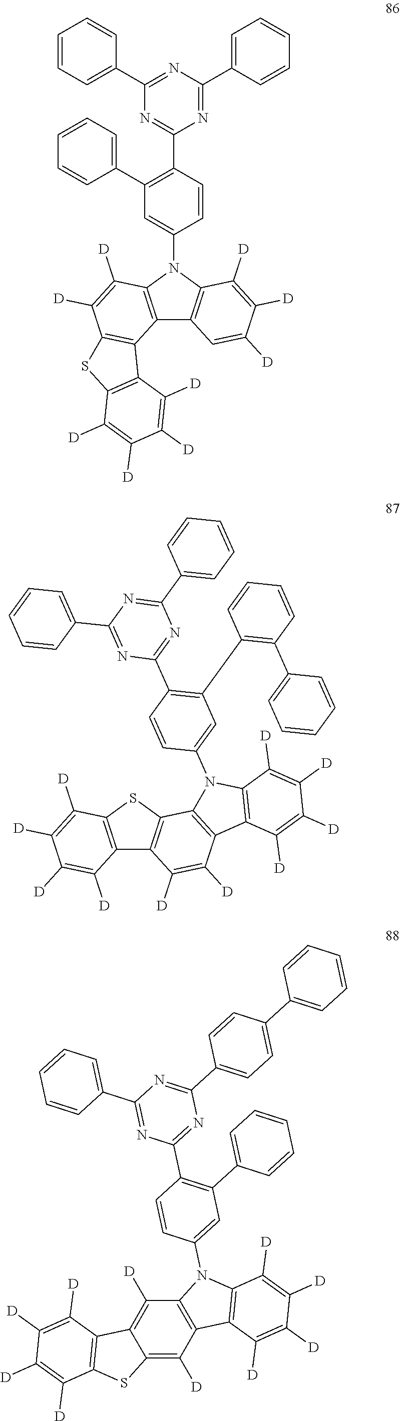 [SMILES: [2H]c1cc2c3c4c(sc5c([2H])c([2H])c([2H])c([2H])c54)c([2H])c([2H])c3n(-c3ccc(-c4nc(-c5ccccc5)nc(-c5ccccc5)n4)c(-c4ccccc4)c3)c2c([2H])c1[2H] ; [2H]c1c([2H])c([2H])c2c(sc3c2c([2H])c([2H])c2c4c([2H])c([2H])c([2H])c([2H])c4n(-c4ccc(-c5nc(-c6ccccc6)nc(-c6ccccc6)n5)c(Cc5ccccc5-c5ccccc5)c4)c32)c1[2H] ; [2H]c1c([2H])c([2H])c2c(sc3c([2H])c4c5c([2H])c([2H])c([2H])c([2H])c5n(-c5ccc(-c6nc(-c7ccccc7)nc(-c7ccc(-c8ccccc8)cc7)n6)c(-c6ccccc6)c5)c4c([2H])c32)c1[2H]]

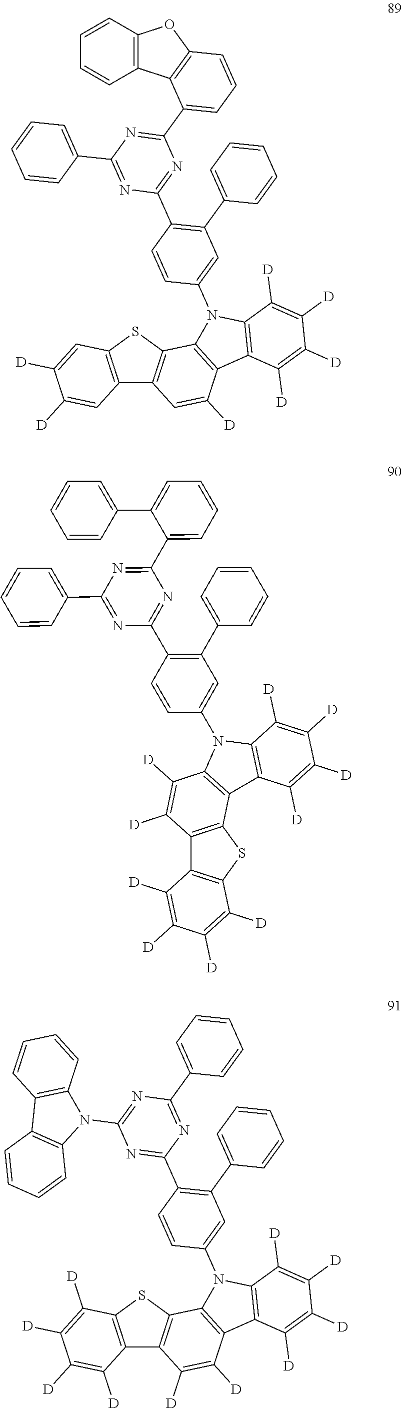 [SMILES: [2H]c1cc2sc3c(cc([2H])c4c5c([2H])c([2H])c([2H])c([2H])c5n(-c5ccc(-c6nc(-c7ccccc7)nc(-c7cccc8oc9ccccc9c78)n6)c(-c6ccccc6)c5)c34)c2cc1[2H] ; [2H]c1c([2H])c([2H])c2c(sc3c2c([2H])c([2H])c2c3c3c([2H])c([2H])c([2H])c([2H])c3n2-c2ccc(-c3nc(-c4ccccc4)nc(-c4ccccc4-c4ccccc4)n3)c(-c3ccccc3)c2)c1[2H] ; [2H]c1c([2H])c([2H])c2c(sc3c2c([2H])c([2H])c2c4c([2H])c([2H])c([2H])c([2H])c4n(-c4ccc(-c5nc(-c6ccccc6)nc(-n6c7ccccc7c7ccccc76)n5)c(-c5ccccc5)c4)c32)c1[2H]]

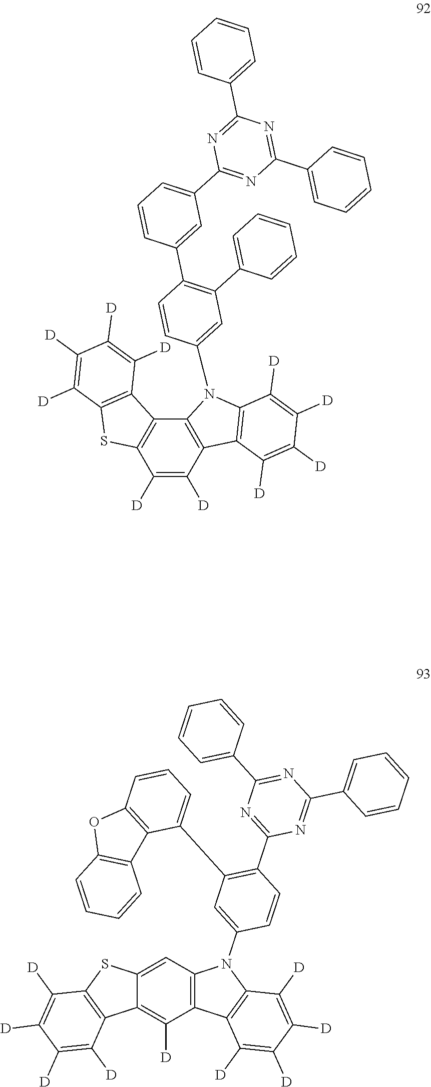 [SMILES: [2H]c1c([2H])c([2H])c2c(sc3c([2H])c([2H])c4c5c([2H])c([2H])c([2H])c([2H])c5n(-c5ccc(-c6cccc(-c7nc(-c8ccccc8)nc(-c8ccccc8)n7)c6)c(-c6ccccc6)c5)c4c32)c1[2H] ; [2H]c1c([2H])c([2H])c2c(sc3cc4c(c([2H])c32)c2c([2H])c([2H])c([2H])c([2H])c2n4-c2ccc(-c3nc(-c4ccccc4)nc(-c4ccccc4)n3)c(Cc3cccc4oc5ccccc5c34)c2)c1[2H]]

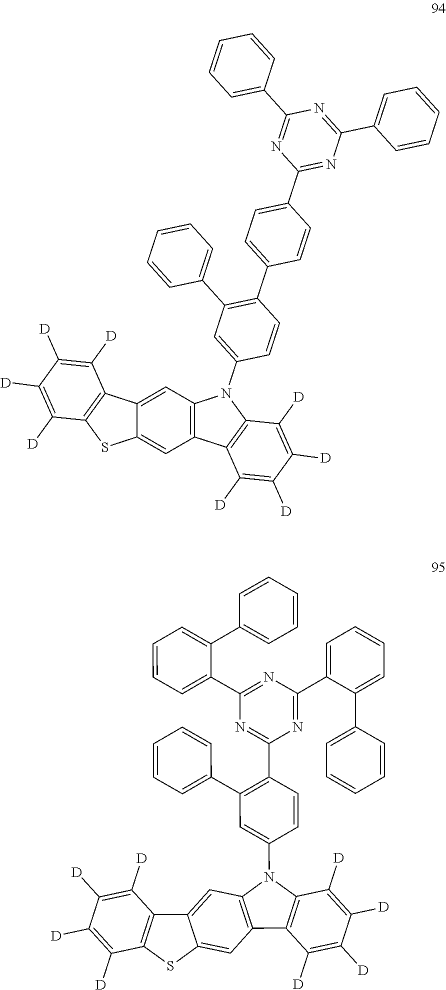 [SMILES: [2H]c1c([2H])c([2H])c2c(sc3cc4c5c([2H])c([2H])c([2H])c([2H])c5n(-c5ccc(-c6ccc(-c7nc(-c8ccccc8)nc(-c8ccccc8)n7)cc6)c(-c6ccccc6)c5)c4cc32)c1[2H] ; [2H]c1c([2H])c([2H])c2c(sc3cc4c5c([2H])c([2H])c([2H])c([2H])c5n(-c5ccc(-c6nc(-c7ccccc7-c7ccccc7)nc(-c7ccccc7-c7ccccc7)n6)c(-c6ccccc6)c5)c4cc32)c1[2H]]

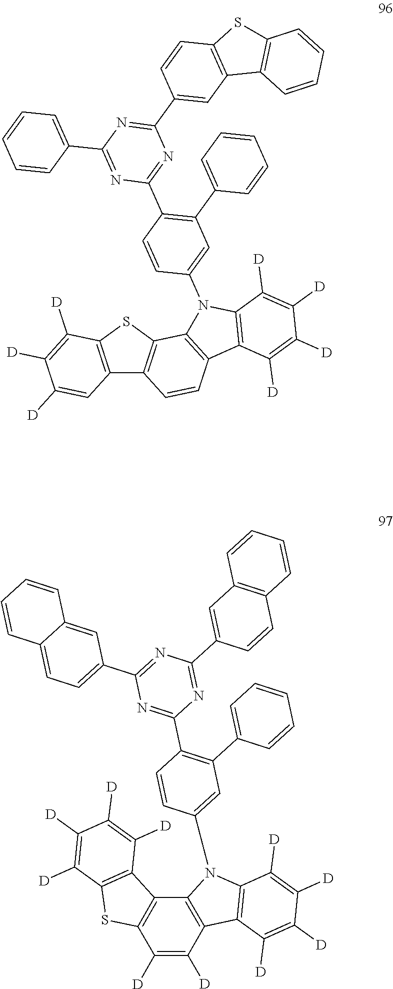 [SMILES: [2H]c1cc2c(sc3c2ccc2c4c([2H])c([2H])c([2H])c([2H])c4n(-c4ccc(-c5nc(-c6ccccc6)nc(-c6ccc7sc8ccccc8c7c6)n5)c(-c5ccccc5)c4)c23)c([2H])c1[2H] ; [2H]c1c([2H])c([2H])c2c(sc3c([2H])c([2H])c4c5c([2H])c([2H])c([2H])c([2H])c5n(-c5ccc(-c6nc(-c7ccc8ccccc8c7)nc(-c7ccc8ccccc8c7)n6)c(-c6ccccc6)c5)c4c32)c1[2H]]

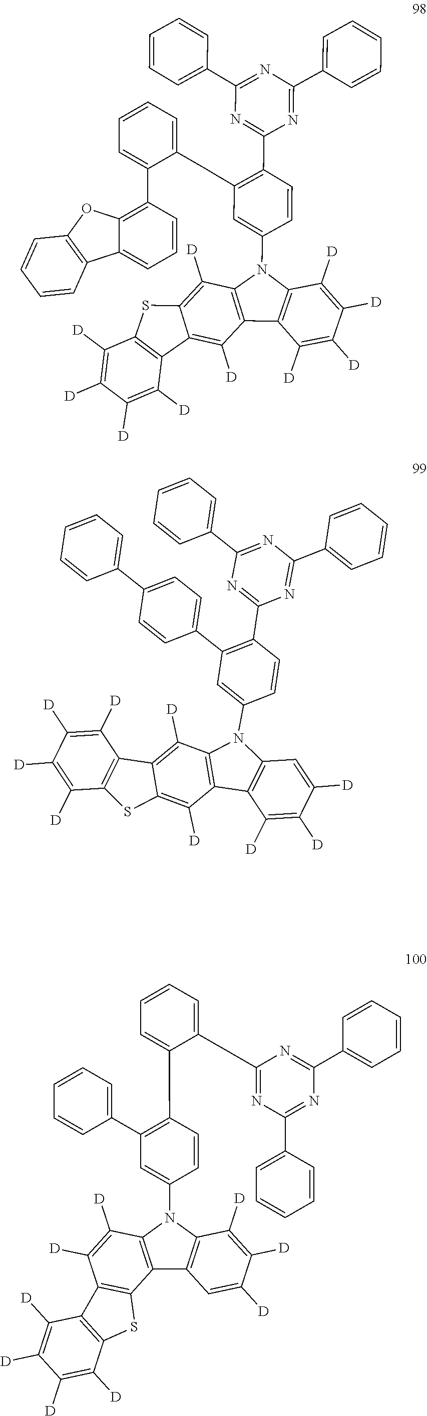 [SMILES: [2H]c1c([2H])c([2H])c2c(sc3c([2H])c4c(c([2H])c32)c2c([2H])c([2H])c([2H])c([2H])c2n4-c2ccc(-c3nc(-c4ccccc4)nc(-c4ccccc4)n3)c(Cc3ccccc3-c3cccc4c3oc3ccccc34)c2)c1[2H] ; [2H]c1cc2c(c([2H])c1[2H])c1c([2H])c3sc4c([2H])c([2H])c([2H])c([2H])c4c3c([2H])c1n2-c1ccc(-c2nc(-c3ccccc3)nc(-c3ccccc3)n2)c(-c2ccc(-c3ccccc3)cc2)c1 ; [2H]c1cc2c3c4sc5c([2H])c([2H])c([2H])c([2H])c5c4c([2H])c([2H])c3n(-c3ccc(Cc4ccccc4Cc4nc(-c5ccccc5)nc(-c5ccccc5)n4)c(-c4ccccc4)c3)c2c([2H])c1[2H]]

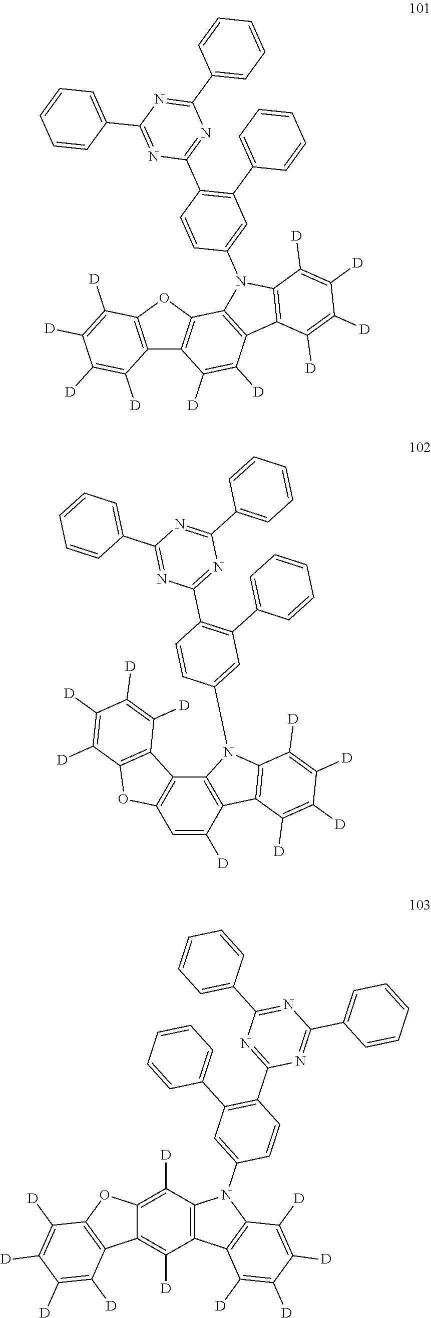 [SMILES: [2H]c1c([2H])c([2H])c2c(oc3c2c([2H])c([2H])c2c4c([2H])c([2H])c([2H])c([2H])c4n(-c4ccc(-c5nc(-c6ccccc6)nc(-c6ccccc6)n5)c(-c5ccccc5)c4)c32)c1[2H] ; [2H]c1c([2H])c([2H])c2c(oc3cc([2H])c4c5c([2H])c([2H])c([2H])c([2H])c5n(Cc5ccc(-c6nc(-c7ccccc7)nc(-c7ccccc7)n6)c(-c6ccccc6)c5)c4c32)c1[2H] ; [2H]c1c([2H])c([2H])c2c(oc3c([2H])c4c(c([2H])c32)c2c([2H])c([2H])c([2H])c([2H])c2n4-c2ccc(-c3nc(-c4ccccc4)nc(-c4ccccc4)n3)c(-c3ccccc3)c2)c1[2H]]

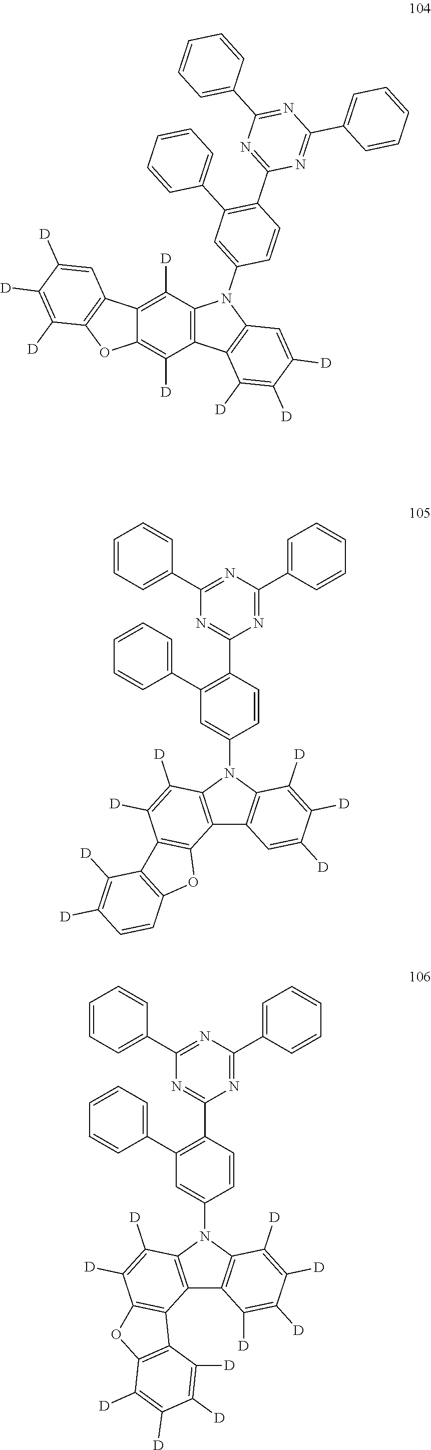 [SMILES: [2H]c1cc2c(oc3c([2H])c4c5c([2H])c([2H])c([2H])cc5n(-c5ccc(-c6nc(-c7ccccc7)nc(-c7ccccc7)n6)c(-c6ccccc6)c5)c4c([2H])c32)c([2H])c1[2H] ; [2H]c1cc2c3c4oc5ccc([2H])c([2H])c5c4c([2H])c([2H])c3n(-c3ccc(-c4nc(-c5ccccc5)nc(-c5ccccc5)n4)c(-c4ccccc4)c3)c2c([2H])c1[2H] ; [2H]c1c([2H])c([2H])c2c(oc3c([2H])c([2H])c4c(c5c([2H])c([2H])c([2H])c([2H])c5n4-c4ccc(-c5nc(-c6ccccc6)nc(-c6ccccc6)n5)c(-c5ccccc5)c4)c32)c1[2H]]

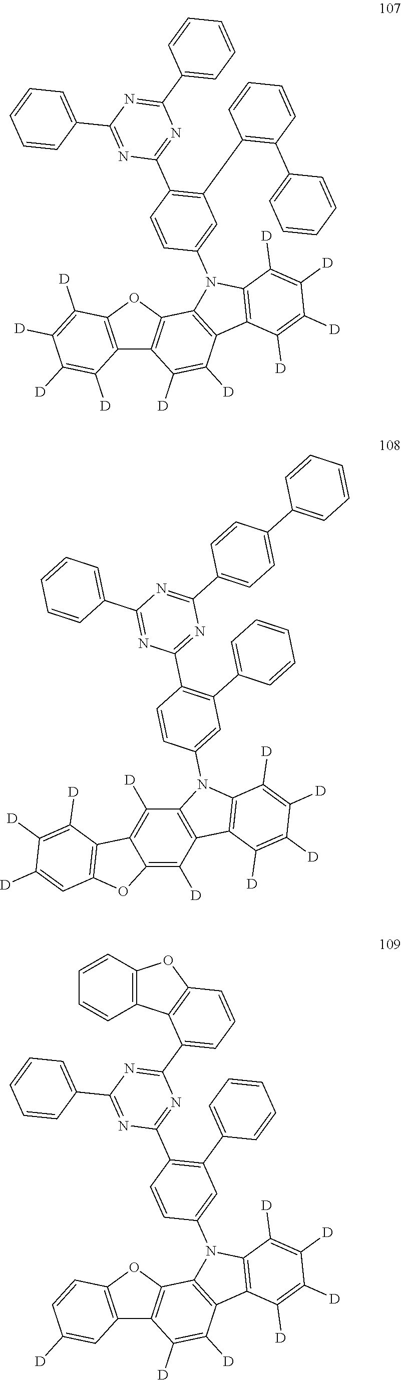 [SMILES: [2H]c1c([2H])c([2H])c2c(oc3c2c([2H])c([2H])c2c4c([2H])c([2H])c([2H])c([2H])c4n(-c4ccc(-c5nc(-c6ccccc6)nc(-c6ccccc6)n5)c(Cc5ccccc5-c5ccccc5)c4)c32)c1[2H] ; [2H]c1cc2oc3c([2H])c4c5c([2H])c([2H])c([2H])c([2H])c5n(-c5ccc(-c6nc(-c7ccccc7)nc(-c7ccc(-c8ccccc8)cc7)n6)c(-c6ccccc6)c5)c4c([2H])c3c2c([2H])c1[2H] ; [2H]c1ccc2oc3c(c([2H])c([2H])c4c5c([2H])c([2H])c([2H])c([2H])c5n(-c5ccc(-c6nc(-c7ccccc7)nc(-c7cccc8oc9ccccc9c78)n6)c(-c6ccccc6)c5)c34)c2c1]

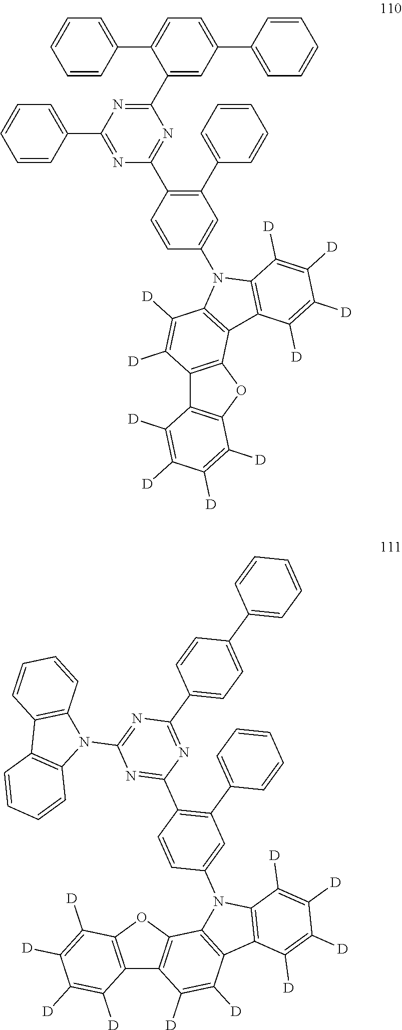 [SMILES: [2H]c1c([2H])c([2H])c2c(oc3c2c([2H])c([2H])c2c3c3c([2H])c([2H])c([2H])c([2H])c3n2-c2ccc(-c3nc(-c4ccccc4)nc(-c4cc(-c5ccccc5)ccc4-c4ccccc4)n3)c(-c3ccccc3)c2)c1[2H] ; [2H]c1c([2H])c([2H])c2c(oc3c2c([2H])c([2H])c2c4c([2H])c([2H])c([2H])c([2H])c4n(-c4ccc(-c5nc(-c6ccc(-c7ccccc7)cc6)nc(-n6c7ccccc7c7ccccc76)n5)c(-c5ccccc5)c4)c32)c1[2H]]

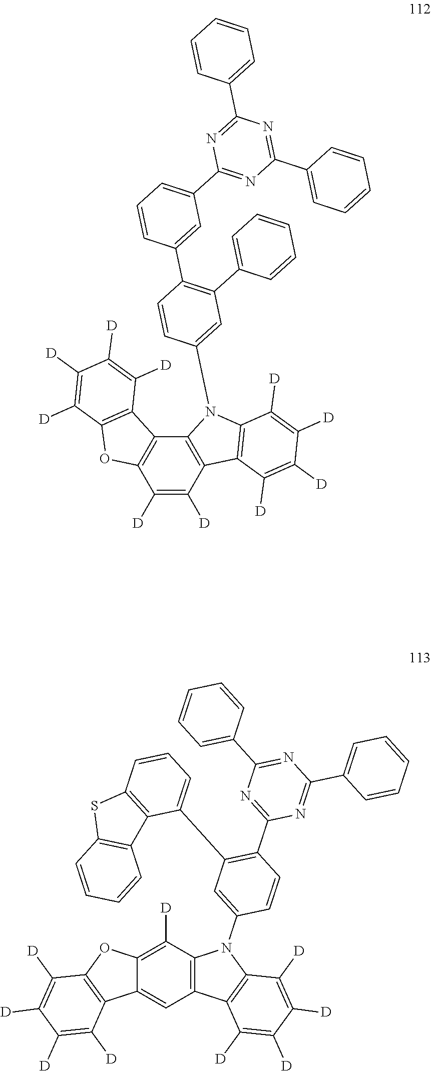 [SMILES: [2H]c1c([2H])c([2H])c2c(oc3c([2H])c([2H])c4c5c([2H])c([2H])c([2H])c([2H])c5n(Cc5ccc(-c6cccc(-c7nc(-c8ccccc8)nc(-c8ccccc8)n7)c6)c(-c6ccccc6)c5)c4c32)c1[2H] ; [2H]c1c([2H])c([2H])c2c(oc3c([2H])c4c(cc32)c2c([2H])c([2H])c([2H])c([2H])c2n4-c2ccc(-c3nc(-c4ccccc4)nc(-c4ccccc4)n3)c(Cc3cccc4sc5ccccc5c34)c2)c1[2H]]

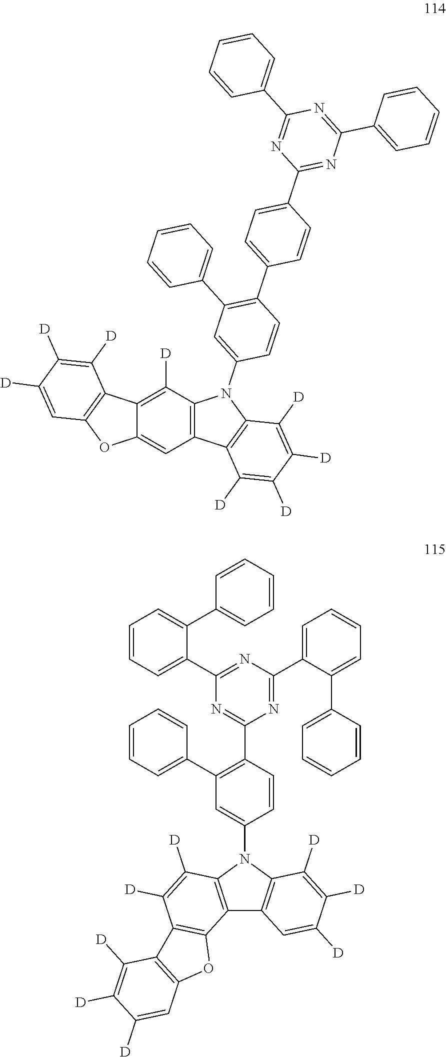 [SMILES: [2H]c1cc2oc3cc4c5c([2H])c([2H])c([2H])c([2H])c5n(-c5ccc(-c6ccc(-c7nc(-c8ccccc8)nc(-c8ccccc8)n7)cc6)c(-c6ccccc6)c5)c4c([2H])c3c2c([2H])c1[2H] ; [2H]c1cc2oc3c(c([2H])c([2H])c4c3c3cc([2H])c([2H])c([2H])c3n4-c3ccc(-c4nc(-c5ccccc5-c5ccccc5)nc(-c5ccccc5-c5ccccc5)n4)c(-c4ccccc4)c3)c2c([2H])c1[2H]]

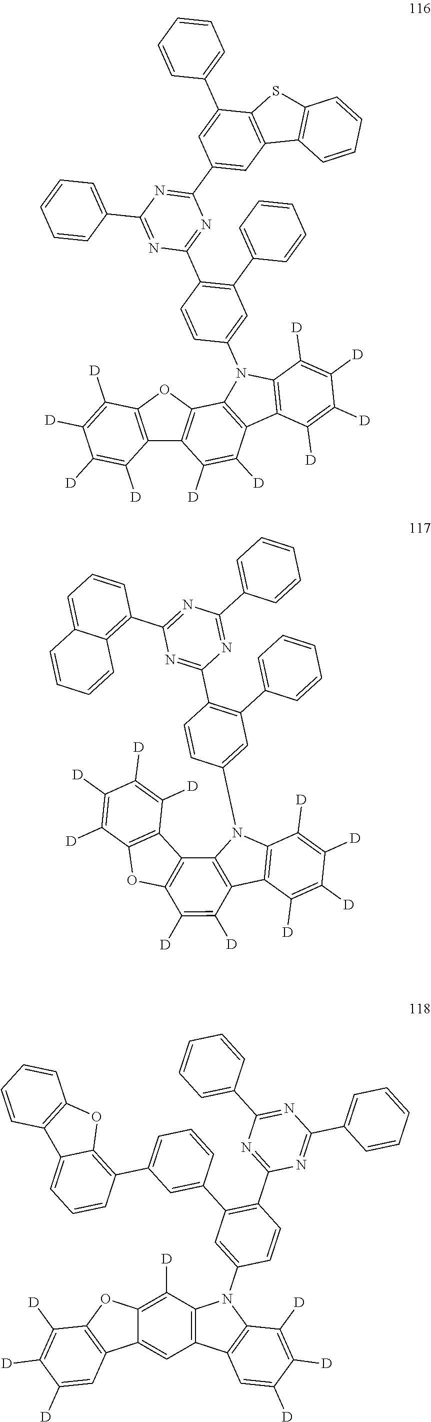 [SMILES: [2H]c1c([2H])c([2H])c2c(oc3c2c([2H])c([2H])c2c4c([2H])c([2H])c([2H])c([2H])c4n(-c4ccc(-c5nc(-c6ccccc6)nc(-c6cc(-c7ccccc7)c7sc8ccccc8c7c6)n5)c(-c5ccccc5)c4)c32)c1[2H] ; [2H]c1c([2H])c([2H])c2c(oc3c([2H])c([2H])c4c5c([2H])c([2H])c([2H])c([2H])c5n(Cc5ccc(-c6nc(-c7ccccc7)nc(-c7cccc8ccccc78)n6)c(-c6ccccc6)c5)c4c32)c1[2H] ; [2H]c1cc2c(oc3c([2H])c4c(cc32)c2cc([2H])c([2H])c([2H])c2n4-c2ccc(-c3nc(-c4ccccc4)nc(-c4ccccc4)n3)c(-c3cccc(-c4cccc5c4oc4ccccc45)c3)c2)c([2H])c1[2H]]

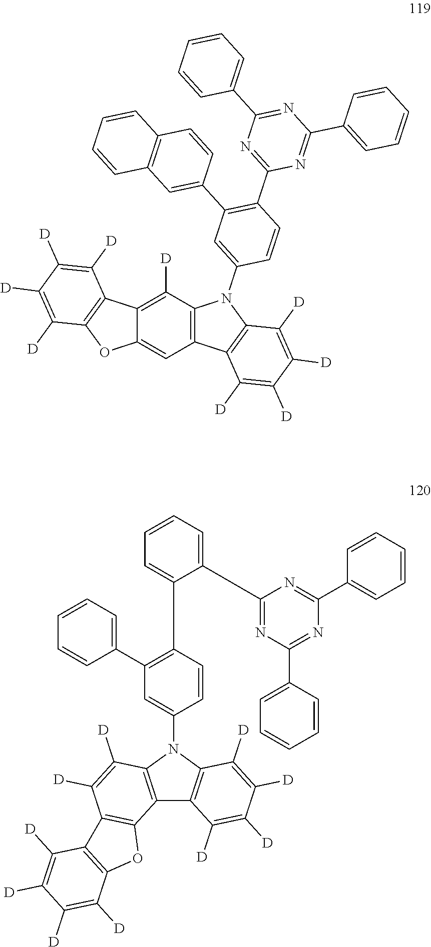 [SMILES: [2H]c1c([2H])c([2H])c2c(oc3cc4c5c([2H])c([2H])c([2H])c([2H])c5n(-c5ccc(-c6nc(-c7ccccc7)nc(-c7ccccc7)n6)c(-c6ccc7ccccc7c6)c5)c4c([2H])c32)c1[2H] ; [2H]c1c([2H])c([2H])c2c(oc3c2c([2H])c([2H])c2c3c3c([2H])c([2H])c([2H])c([2H])c3n2-c2ccc(Cc3ccccc3Cc3nc(-c4ccccc4)nc(-c4ccccc4)n3)c(-c3ccccc3)c2)c1[2H]]

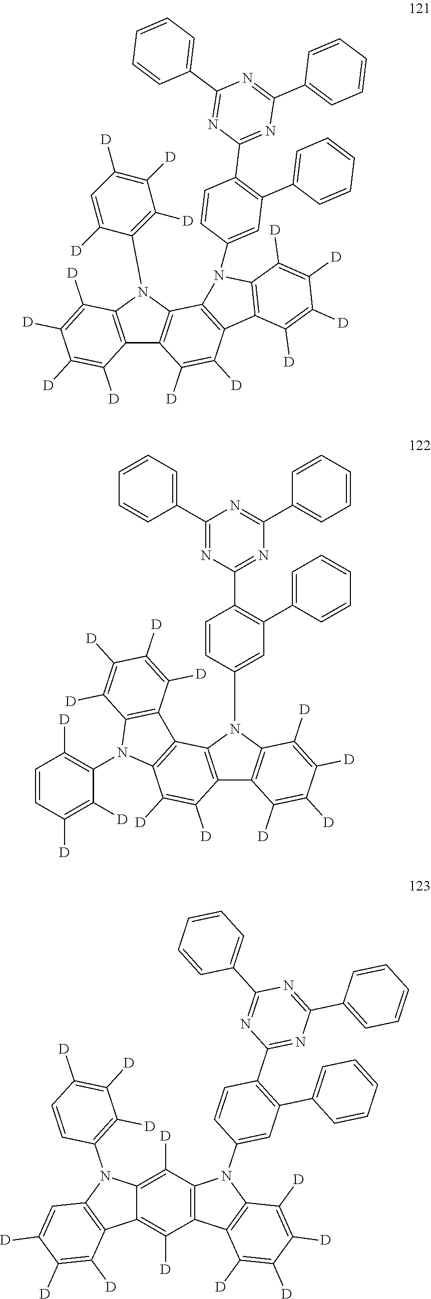

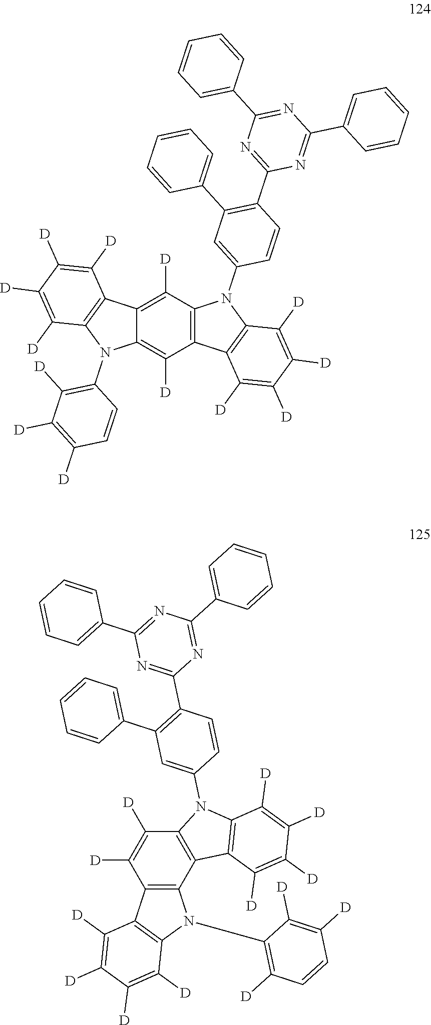 [SMILES: [2H]c1ccc(-n2c3c([2H])c([2H])c([2H])c([2H])c3c3c([2H])c4c(c([2H])c32)c2c([2H])c([2H])c([2H])c([2H])c2n4-c2ccc(-c3nc(-c4ccccc4)nc(-c4ccccc4)n3)c(-c3ccccc3)c2)c([2H])c1[2H] ; [2H]c1ccc([2H])c(-n2c3c([2H])c([2H])c([2H])c([2H])c3c3c([2H])c([2H])c4c(c5c([2H])c([2H])c([2H])c([2H])c5n4-c4ccc(-c5nc(-c6ccccc6)nc(-c6ccccc6)n5)c(-c5ccccc5)c4)c32)c1[2H]]

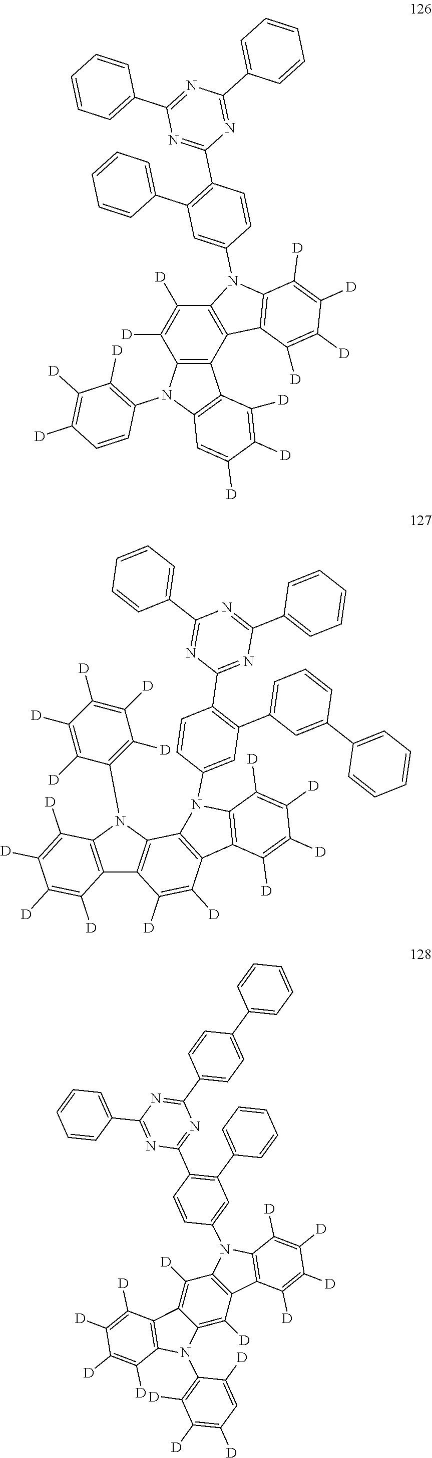

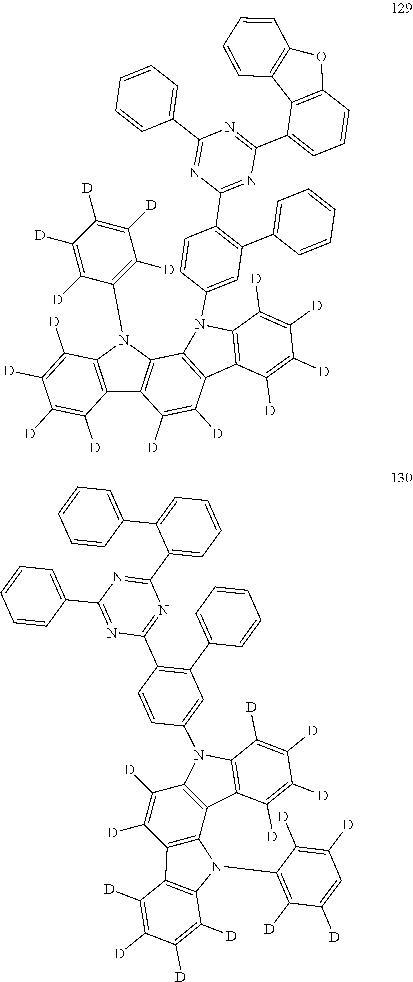

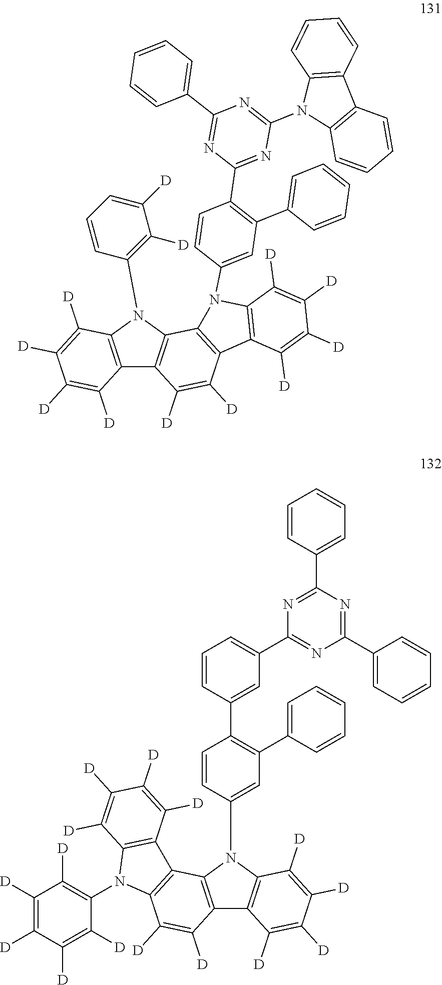 [SMILES: [2H]c1cccc(-n2c3c([2H])c([2H])c([2H])c([2H])c3c3c([2H])c([2H])c4c5c([2H])c([2H])c([2H])c([2H])c5n(-c5ccc(-c6nc(-c7ccccc7)nc(-n7c8ccccc8c8ccccc87)n6)c(-c6ccccc6)c5)c4c32)c1[2H] ; [2H]c1c([2H])c([2H])c(-n2c3c([2H])c([2H])c([2H])c([2H])c3c3c2c([2H])c([2H])c2c4c([2H])c([2H])c([2H])c([2H])c4n(-c4ccc(-c5cccc(-c6nc(-c7ccccc7)nc(-c7ccccc7)n6)c5)c(-c5ccccc5)c4)c23)c([2H])c1[2H]]

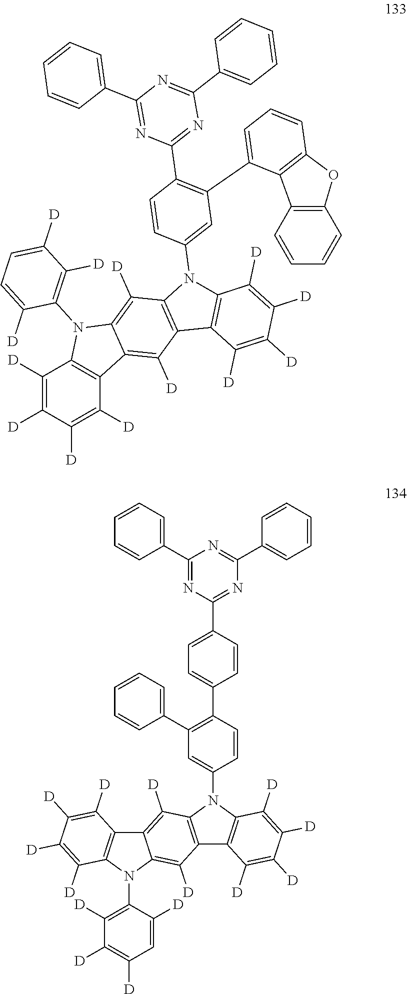 [SMILES: [2H]c1ccc([2H])c(-n2c3c([2H])c([2H])c([2H])c([2H])c3c3c([2H])c4c5c([2H])c([2H])c([2H])c([2H])c5n(-c5ccc(-c6nc(-c7ccccc7)nc(-c7ccccc7)n6)c(-c6cccc7oc8ccccc8c67)c5)c4c([2H])c32)c1[2H] ; [2H]c1cc([2H])c(-n2c3c([2H])c([2H])c([2H])c([2H])c3c3c([2H])c4c(c([2H])c32)c2c([2H])c([2H])c([2H])c([2H])c2n4-c2ccc(-c3ccc(-c4nc(-c5ccccc5)nc(-c5ccccc5)n4)cc3)c(-c3ccccc3)c2)c([2H])c1[2H]]

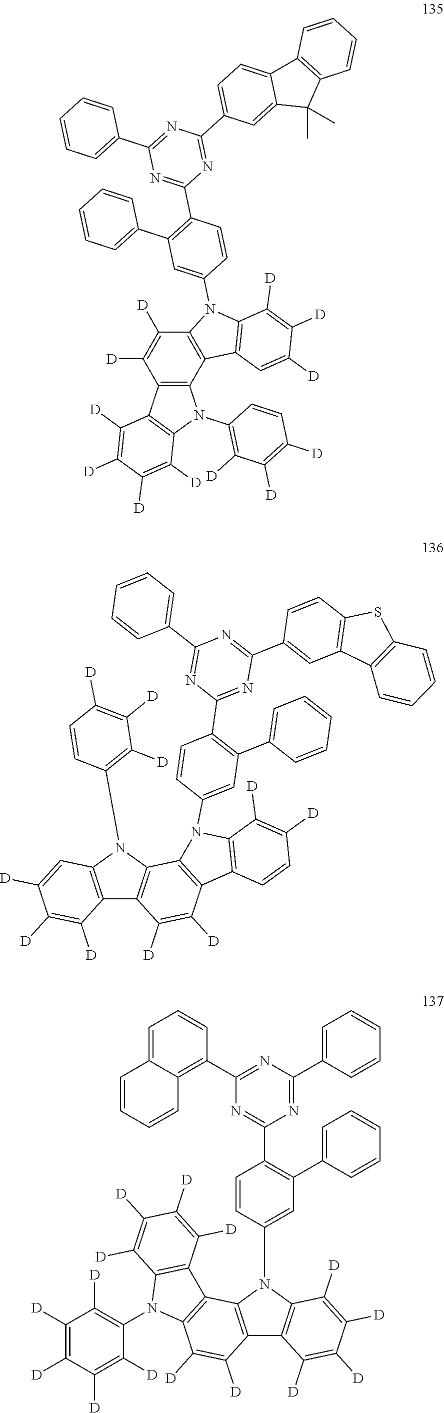

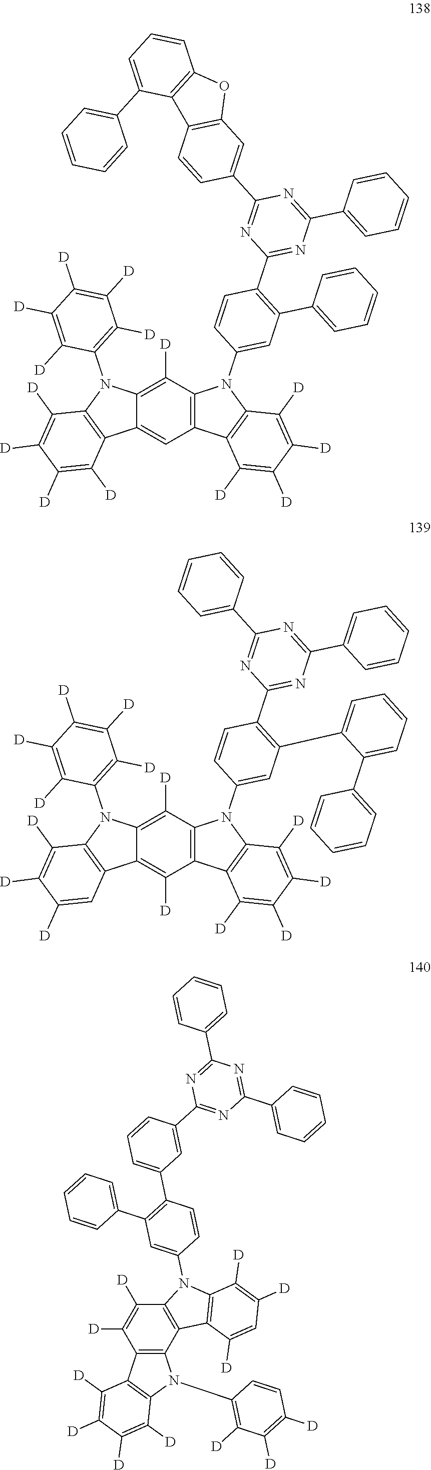

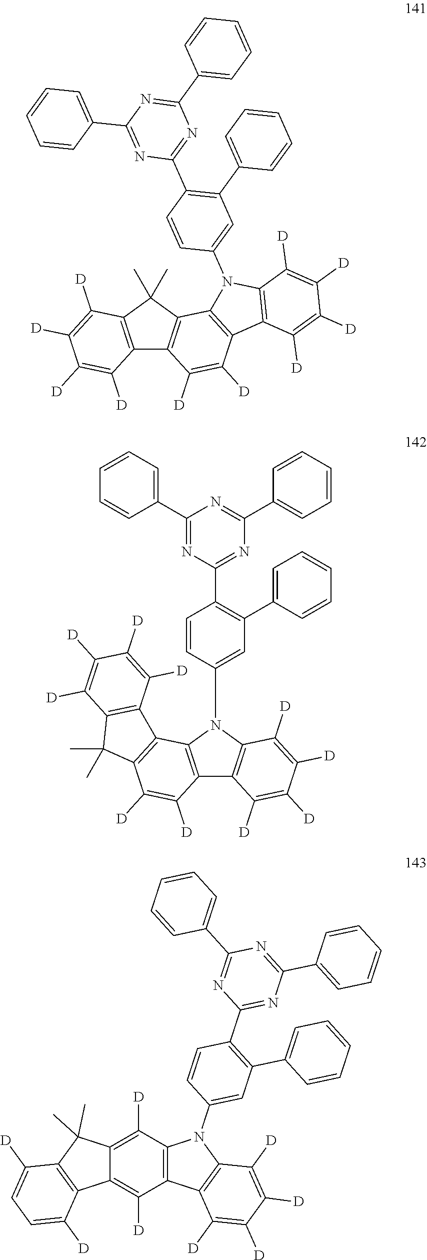

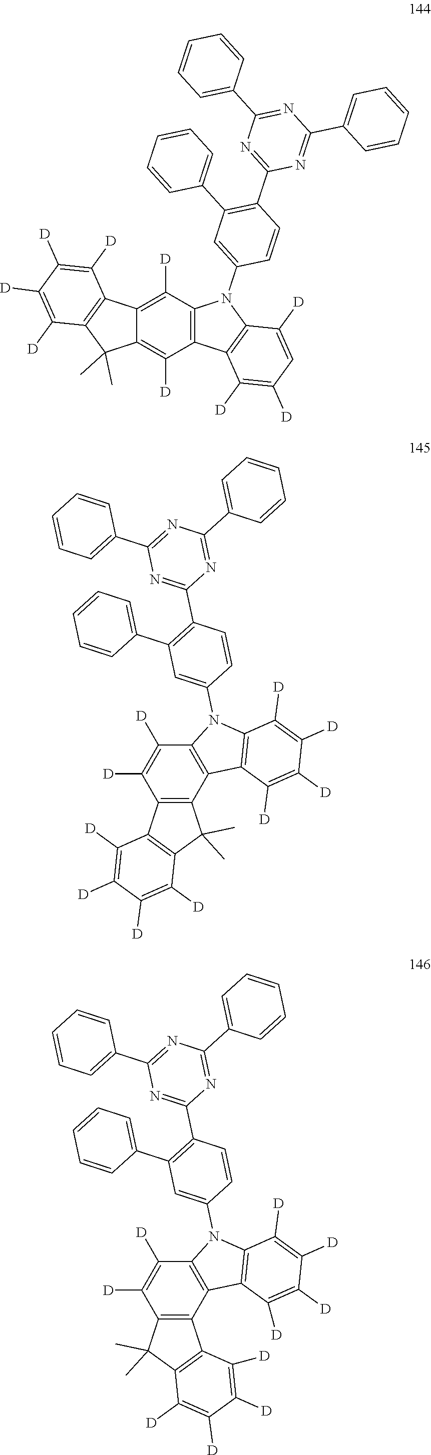 [SMILES: [2H]c1cc([2H])c2c(c1[2H])c1c([2H])c3c(c([2H])c1n2-c1ccc(-c2nc(-c4ccccc4)nc(-c4ccccc4)n2)c(-c2ccccc2)c1)-c1c([2H])c([2H])c([2H])c([2H])c1C3(C)C ; [2H]c1c([2H])c([2H])c2c(c1[2H])-c1c([2H])c([2H])c3c(c1C2(C)C)c1c([2H])c([2H])c([2H])c([2H])c1n3-c1ccc(-c2nc(-c3ccccc3)nc(-c3ccccc3)n2)c(-c2ccccc2)c1 ; [2H]c1c([2H])c([2H])c2c(c1[2H])-c1c(c([2H])c([2H])c3c1c1c([2H])c([2H])c([2H])c([2H])c1n3-c1ccc(-c3nc(-c4ccccc4)nc(-c4ccccc4)n3)c(-c3ccccc3)c1)C2(C)C]

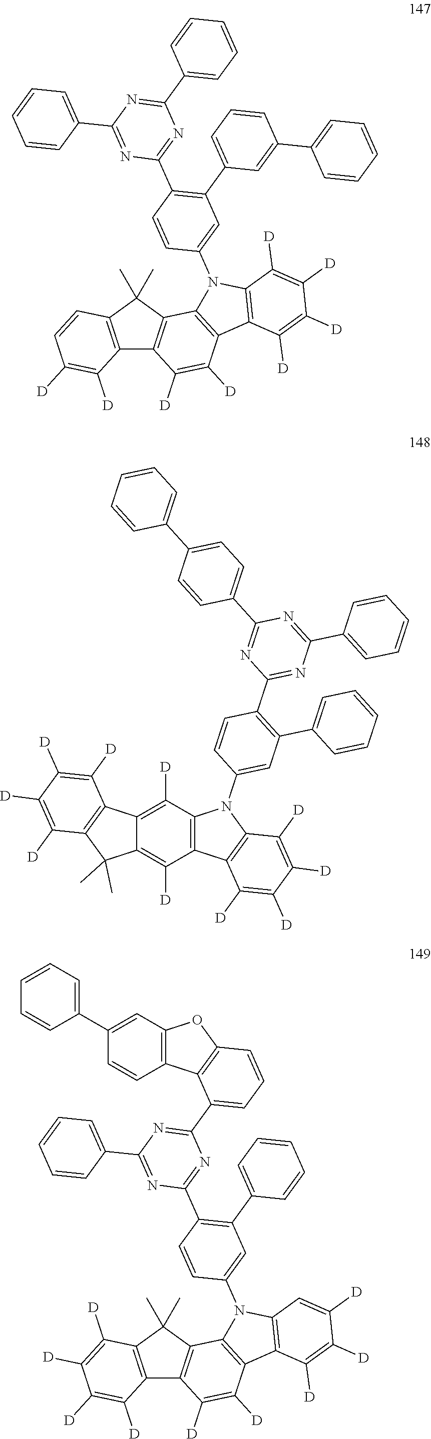 [SMILES: [2H]c1ccc2c(c1[2H])-c1c([2H])c([2H])c3c4c([2H])c([2H])c([2H])c([2H])c4n(-c4ccc(-c5nc(-c6ccccc6)nc(-c6ccccc6)n5)c(-c5cccc(-c6ccccc6)c5)c4)c3c1C2(C)C ; [2H]c1c([2H])c([2H])c2c(c1[2H])-c1c(c([2H])c3c4c([2H])c([2H])c([2H])c([2H])c4n(-c4ccc(-c5nc(-c6ccccc6)nc(-c6ccc(-c7ccccc7)cc6)n5)c(-c5ccccc5)c4)c3c1[2H])C2(C)C ; [2H]c1cc2c(c([2H])c1[2H])c1c([2H])c([2H])c3c(c1n2-c1ccc(-c2nc(-c4ccccc4)nc(-c4cccc5oc6cc(-c7ccccc7)ccc6c45)n2)c(-c2ccccc2)c1)C(C)(C)c1c([2H])c([2H])c([2H])c([2H])c1-3]

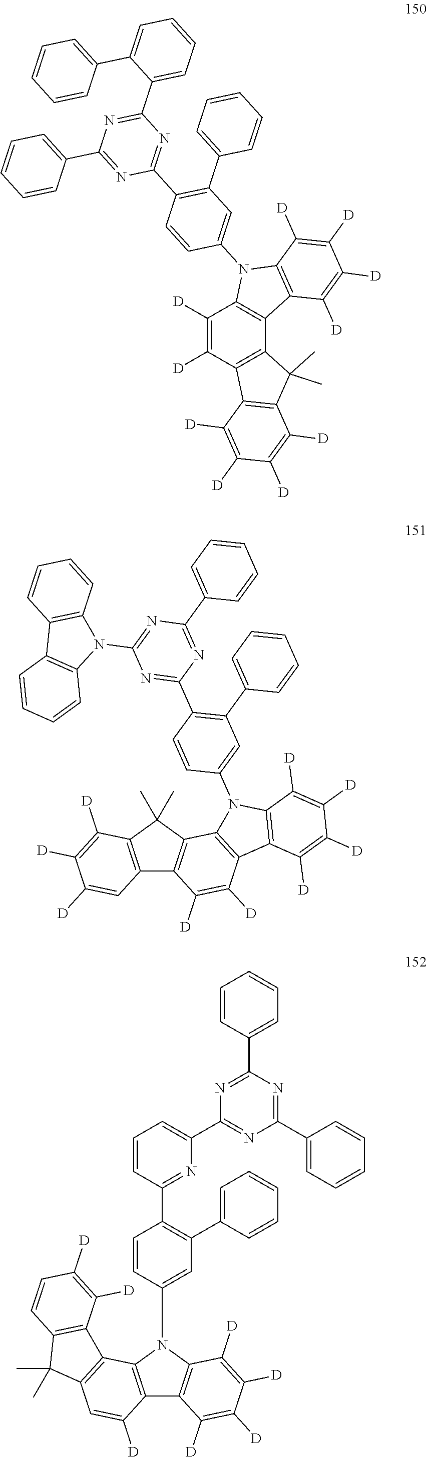 [SMILES: [2H]c1c([2H])c([2H])c2c(c1[2H])-c1c([2H])c([2H])c3c(c1C2(C)C)c1c([2H])c([2H])c([2H])c([2H])c1n3-c1ccc(-c2nc(-c3ccccc3)nc(-c3ccccc3-c3ccccc3)n2)c(-c2ccccc2)c1 ; [2H]c1cc2c(c([2H])c1[2H])C(C)(C)c1c-2c([2H])c([2H])c2c3c([2H])c([2H])c([2H])c([2H])c3n(-c3ccc(-c4nc(-c5ccccc5)nc(-n5c6ccccc6c6ccccc65)n4)c(-c4ccccc4)c3)c12 ; [2H]c1ccc2c(c1[2H])-c1c(cc([2H])c3c4c([2H])c([2H])c([2H])c([2H])c4n(-c4ccc(-c5cccc(-c6nc(-c7ccccc7)nc(-c7ccccc7)n6)n5)c(-c5ccccc5)c4)c13)C2(C)C]

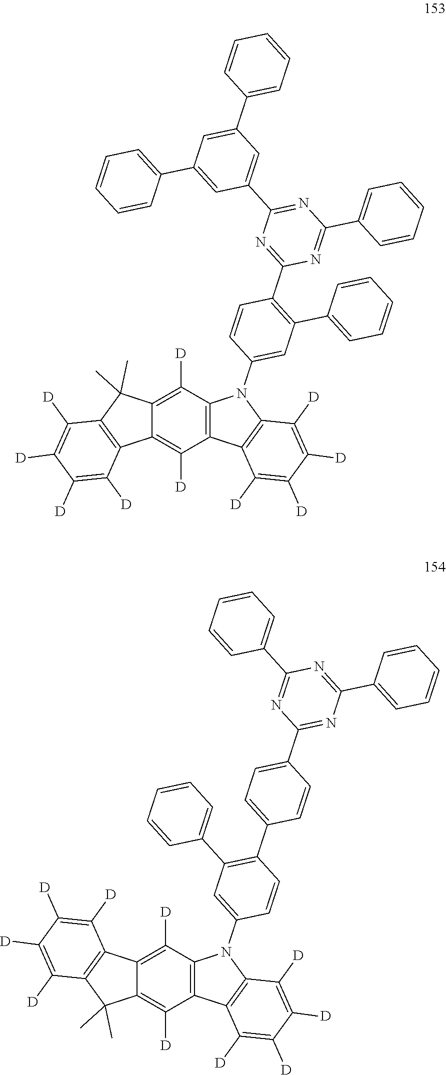 [SMILES: [2H]c1c([2H])c([2H])c2c(c1[2H])-c1c(c([2H])c3c(c1[2H])c1c([2H])c([2H])c([2H])c([2H])c1n3-c1ccc(-c3nc(-c4ccccc4)nc(-c4cc(-c5ccccc5)cc(-c5ccccc5)c4)n3)c(-c3ccccc3)c1)C2(C)C ; [2H]c1c([2H])c([2H])c2c(c1[2H])-c1c(c([2H])c3c4c([2H])c([2H])c([2H])c([2H])c4n(-c4ccc(-c5ccc(-c6nc(-c7ccccc7)nc(-c7ccccc7)n6)cc5)c(-c5ccccc5)c4)c3c1[2H])C2(C)C]

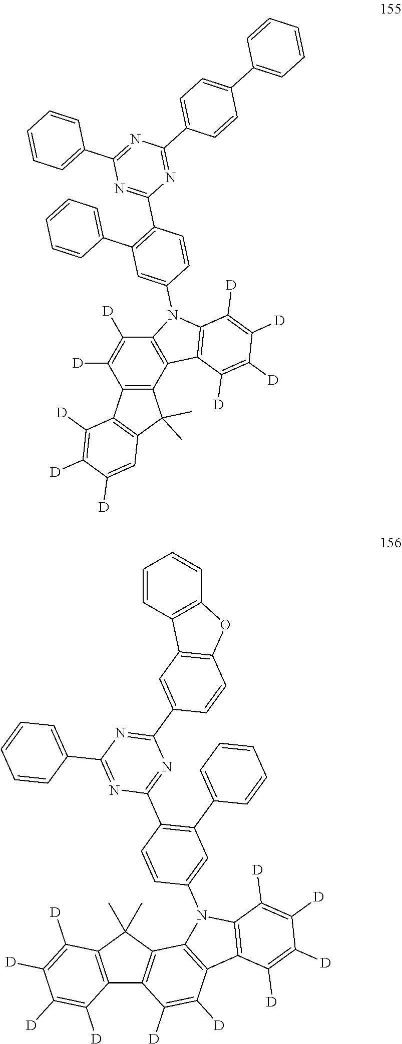 [SMILES: [2H]c1cc2c(c([2H])c1[2H])-c1c([2H])c([2H])c3c(c1C2(C)C)c1c([2H])c([2H])c([2H])c([2H])c1n3-c1ccc(-c2nc(-c3ccccc3)nc(-c3ccc(-c4ccccc4)cc3)n2)c(-c2ccccc2)c1 ; [2H]c1c([2H])c([2H])c2c(c1[2H])-c1c([2H])c([2H])c3c4c([2H])c([2H])c([2H])c([2H])c4n(-c4ccc(-c5nc(-c6ccccc6)nc(-c6ccc7oc8ccccc8c7c6)n5)c(-c5ccccc5)c4)c3c1C2(C)C]

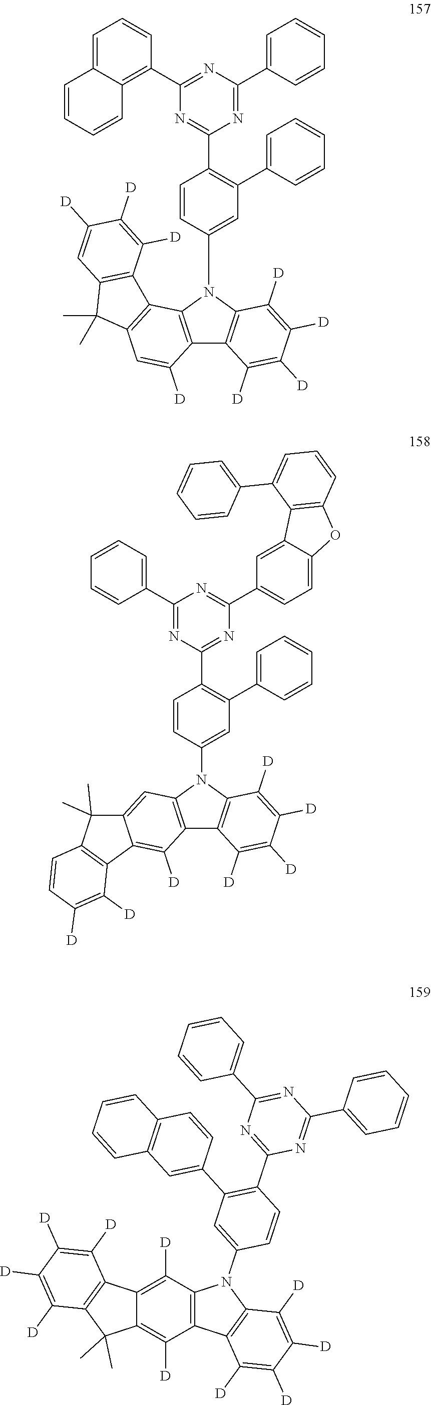 [SMILES: [2H]c1cc2c(c([2H])c1[2H])-c1c(cc([2H])c3c4c([2H])c([2H])c([2H])c([2H])c4n(-c4ccc(-c5nc(-c6ccccc6)nc(-c6cccc7ccccc67)n5)c(-c5ccccc5)c4)c13)C2(C)C ; [2H]c1ccc2c(c1[2H])-c1c(cc3c(c1[2H])c1c([2H])c([2H])c([2H])c([2H])c1n3-c1ccc(-c3nc(-c4ccccc4)nc(-c4ccc5oc6cccc(-c7ccccc7)c6c5c4)n3)c(-c3ccccc3)c1)C2(C)C ; [2H]c1c([2H])c([2H])c2c(c1[2H])-c1c(c([2H])c3c4c([2H])c([2H])c([2H])c([2H])c4n(-c4ccc(-c5nc(-c6ccccc6)nc(-c6ccccc6)n5)c(-c5ccc6ccccc6c5)c4)c3c1[2H])C2(C)C]

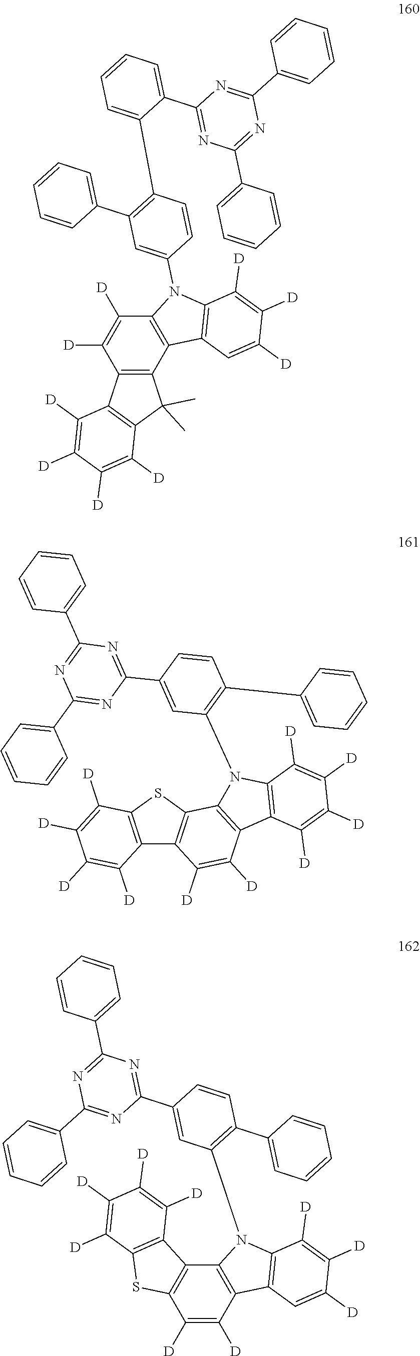 [SMILES: [2H]c1cc2c3c4c(c([2H])c([2H])c3n(-c3ccc(-c5ccccc5-c5nc(-c6ccccc6)nc(-c6ccccc6)n5)c(-c5ccccc5)c3)c2c([2H])c1[2H])-c1c([2H])c([2H])c([2H])c([2H])c1C4(C)C ; [2H]c1c([2H])c([2H])c2c(sc3c2c([2H])c([2H])c2c4c([2H])c([2H])c([2H])c([2H])c4n(-c4cc(-c5nc(-c6ccccc6)nc(-c6ccccc6)n5)ccc4-c4ccccc4)c32)c1[2H] ; [2H]c1cc2c3c([2H])c([2H])c4sc5c([2H])c([2H])c([2H])c([2H])c5c4c3n(-c3cc(-c4nc(-c5ccccc5)nc(-c5ccccc5)n4)ccc3-c3ccccc3)c2c([2H])c1[2H]]

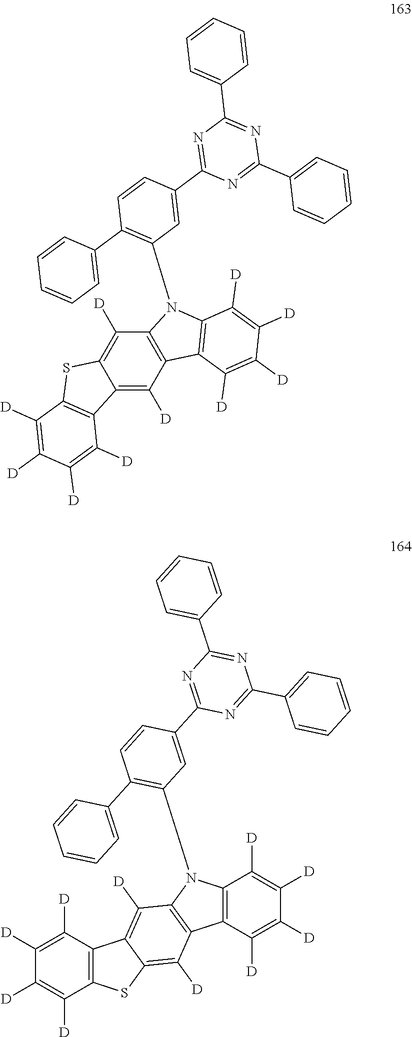 [SMILES: [2H]c1c([2H])c([2H])c2c(sc3c([2H])c4c(c([2H])c32)c2c([2H])c([2H])c([2H])c([2H])c2n4-c2cc(-c3nc(-c4ccccc4)nc(-c4ccccc4)n3)ccc2-c2ccccc2)c1[2H] ; [2H]c1c([2H])c([2H])c2c(sc3c([2H])c4c5c([2H])c([2H])c([2H])c([2H])c5n(-c5cc(-c6nc(-c7ccccc7)nc(-c7ccccc7)n6)ccc5-c5ccccc5)c4c([2H])c32)c1[2H]]

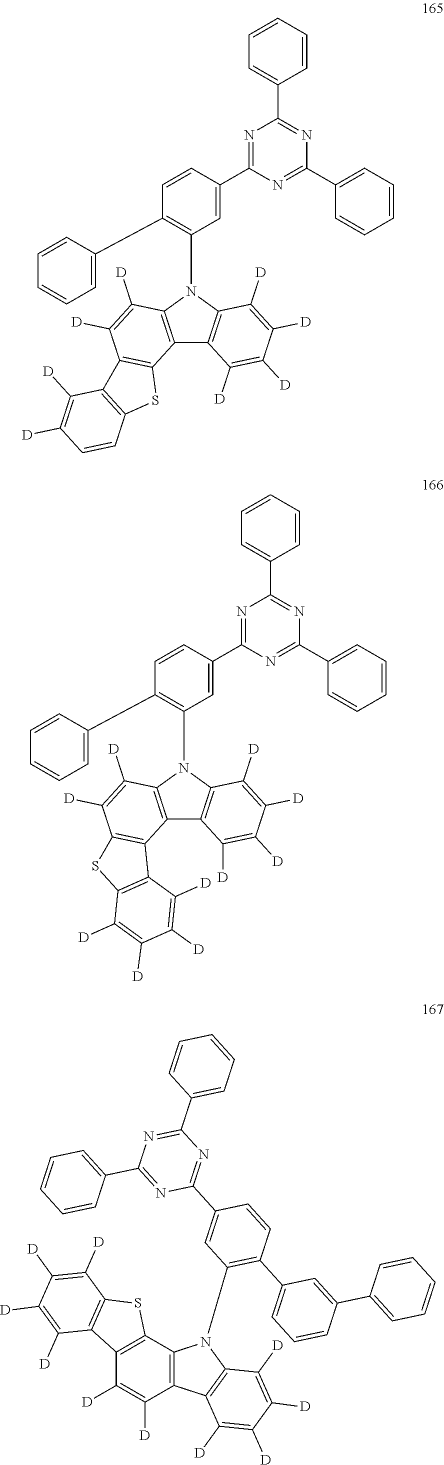 [SMILES: [2H]c1ccc2sc3c(c([2H])c([2H])c4c3c3c([2H])c([2H])c([2H])c([2H])c3n4-c3cc(-c4nc(-c5ccccc5)nc(-c5ccccc5)n4)ccc3-c3ccccc3)c2c1[2H] ; [2H]c1c([2H])c([2H])c2c(sc3c([2H])c([2H])c4c(c5c([2H])c([2H])c([2H])c([2H])c5n4-c4cc(-c5nc(-c6ccccc6)nc(-c6ccccc6)n5)ccc4-c4ccccc4)c32)c1[2H] ; [2H]c1c([2H])c([2H])c2c(sc3c2c([2H])c([2H])c2c4c([2H])c([2H])c([2H])c([2H])c4n(-c4cc(-c5nc(-c6ccccc6)nc(-c6ccccc6)n5)ccc4-c4cccc(-c5ccccc5)c4)c32)c1[2H]]

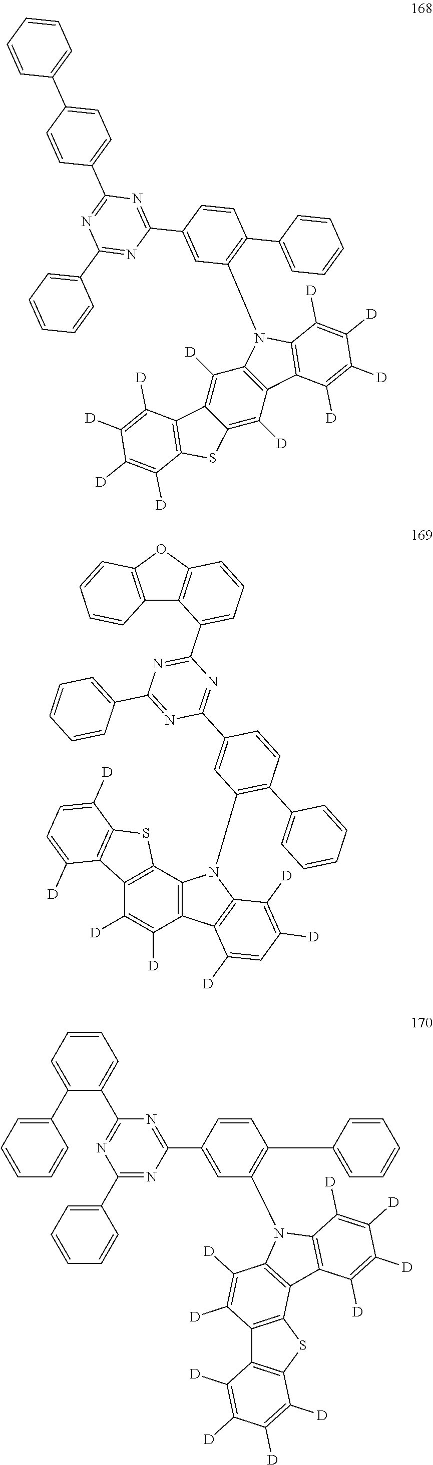

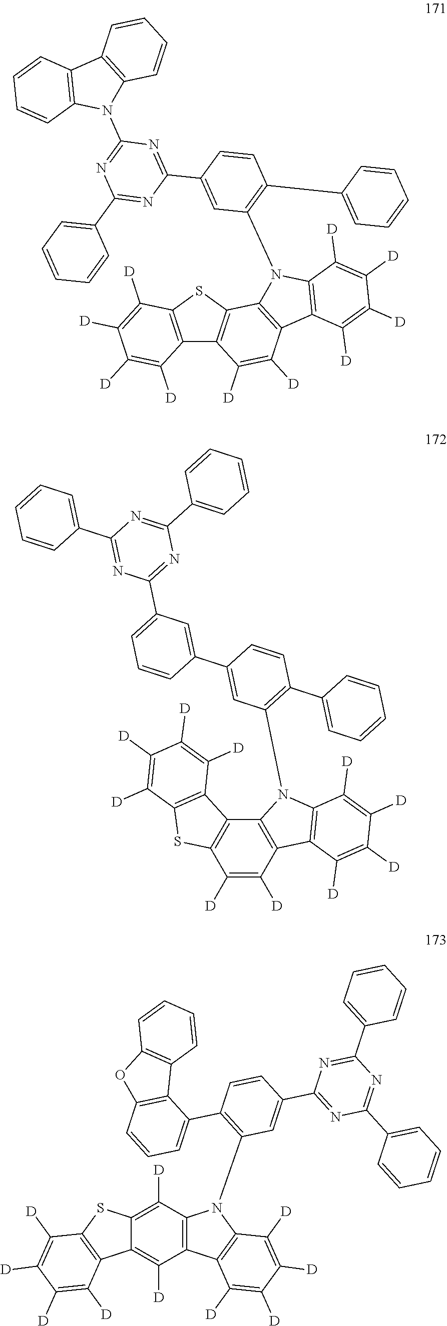 [SMILES: [2H]c1c([2H])c([2H])c2c(sc3c2c([2H])c([2H])c2c4c([2H])c([2H])c([2H])c([2H])c4n(-c4cc(-c5nc(-c6ccccc6)nc(-n6c7ccccc7c7ccccc76)n5)ccc4-c4ccccc4)c32)c1[2H] ; [2H]c1c([2H])c([2H])c2c(sc3c([2H])c([2H])c4c5c([2H])c([2H])c([2H])c([2H])c5n(-c5cc(-c6cccc(-c7nc(-c8ccccc8)nc(-c8ccccc8)n7)c6)ccc5-c5ccccc5)c4c32)c1[2H] ; [2H]c1c([2H])c([2H])c2c(sc3c([2H])c4c(c([2H])c32)c2c([2H])c([2H])c([2H])c([2H])c2n4-c2cc(-c3nc(-c4ccccc4)nc(-c4ccccc4)n3)ccc2-c2cccc3oc4ccccc4c23)c1[2H]]

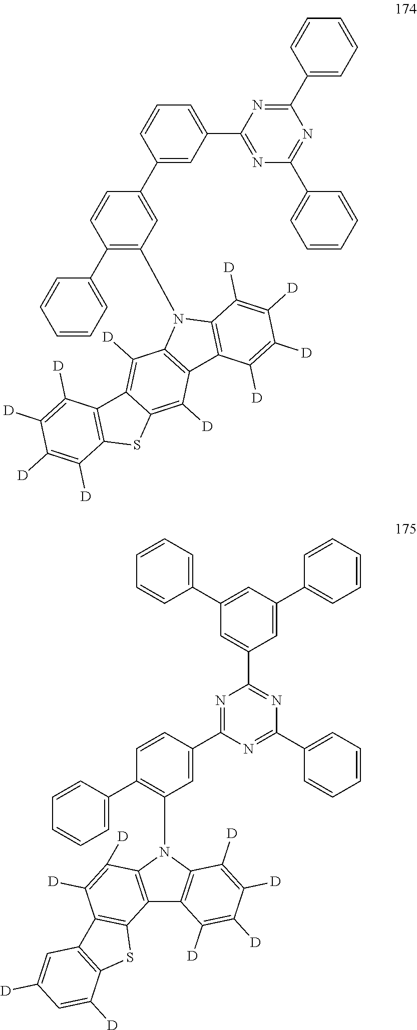 [SMILES: [2H]c1c([2H])c([2H])c2c(sc3c([2H])c4c5c([2H])c([2H])c([2H])c([2H])c5n(-c5cc(-c6cccc(-c7nc(-c8ccccc8)nc(-c8ccccc8)n7)c6)ccc5-c5ccccc5)c4c([2H])c32)c1[2H] ; [2H]c1cc([2H])c2sc3c(c([2H])c([2H])c4c3c3c([2H])c([2H])c([2H])c([2H])c3n4-c3cc(-c4nc(-c5ccccc5)nc(-c5cc(-c6ccccc6)cc(-c6ccccc6)c5)n4)ccc3-c3ccccc3)c2c1]

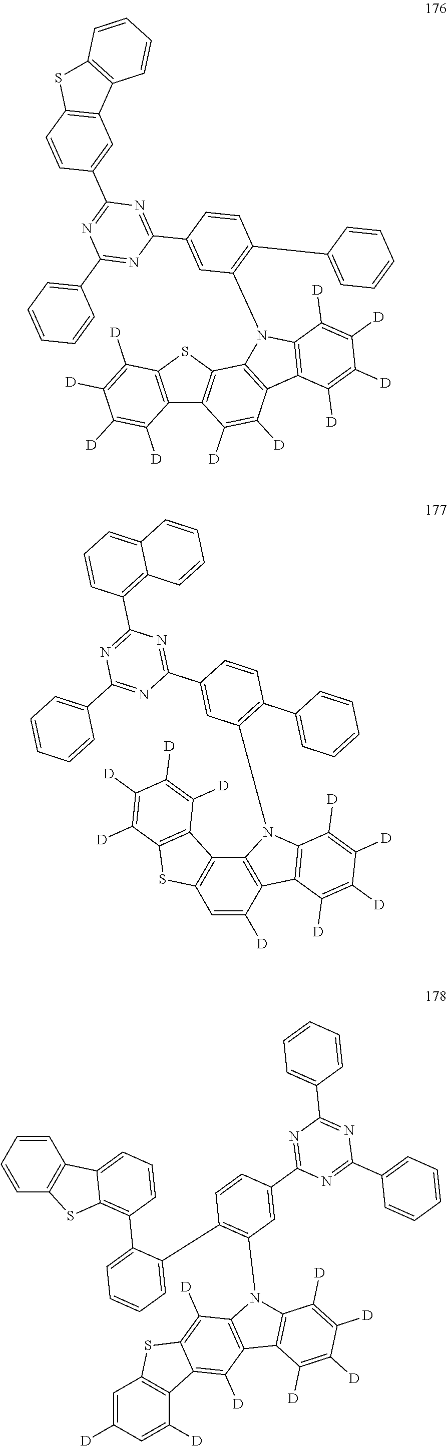 [SMILES: [2H]c1c([2H])c([2H])c2c(sc3c2c([2H])c([2H])c2c4c([2H])c([2H])c([2H])c([2H])c4n(-c4cc(-c5nc(-c6ccccc6)nc(-c6ccc7sc8ccccc8c7c6)n5)ccc4-c4ccccc4)c32)c1[2H] ; [2H]c1c([2H])c([2H])c2c(sc3cc([2H])c4c5c([2H])c([2H])c([2H])c([2H])c5n(-c5cc(-c6nc(-c7ccccc7)nc(-c7cccc8ccccc78)n6)ccc5-c5ccccc5)c4c32)c1[2H] ; [2H]c1cc([2H])c2c(c1)sc1c([2H])c3c(c([2H])c12)c1c([2H])c([2H])c([2H])c([2H])c1n3-c1cc(-c2nc(-c3ccccc3)nc(-c3ccccc3)n2)ccc1-c1ccccc1-c1cccc2c1sc1ccccc12]

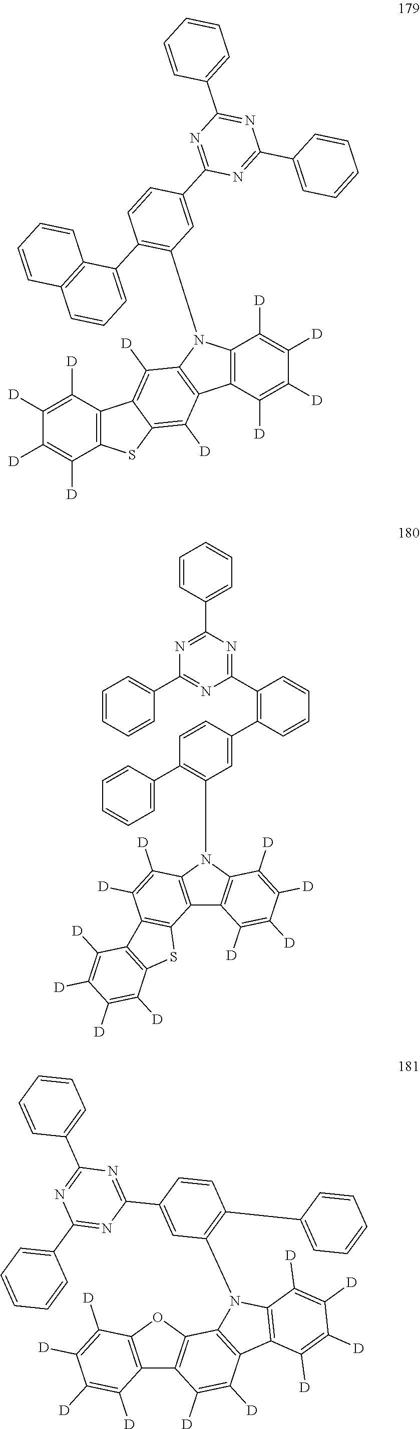 [SMILES: [2H]c1c([2H])c([2H])c2c(sc3c([2H])c4c5c([2H])c([2H])c([2H])c([2H])c5n(-c5cc(-c6nc(-c7ccccc7)nc(-c7ccccc7)n6)ccc5-c5cccc6ccccc56)c4c([2H])c32)c1[2H] ; [2H]c1c([2H])c([2H])c2c(sc3c2c([2H])c([2H])c2c3c3c([2H])c([2H])c([2H])c([2H])c3n2-c2cc(-c3ccccc3-c3nc(-c4ccccc4)nc(-c4ccccc4)n3)ccc2-c2ccccc2)c1[2H] ; [2H]c1c([2H])c([2H])c2c(oc3c2c([2H])c([2H])c2c4c([2H])c([2H])c([2H])c([2H])c4n(-c4cc(-c5nc(-c6ccccc6)nc(-c6ccccc6)n5)ccc4-c4ccccc4)c32)c1[2H]]

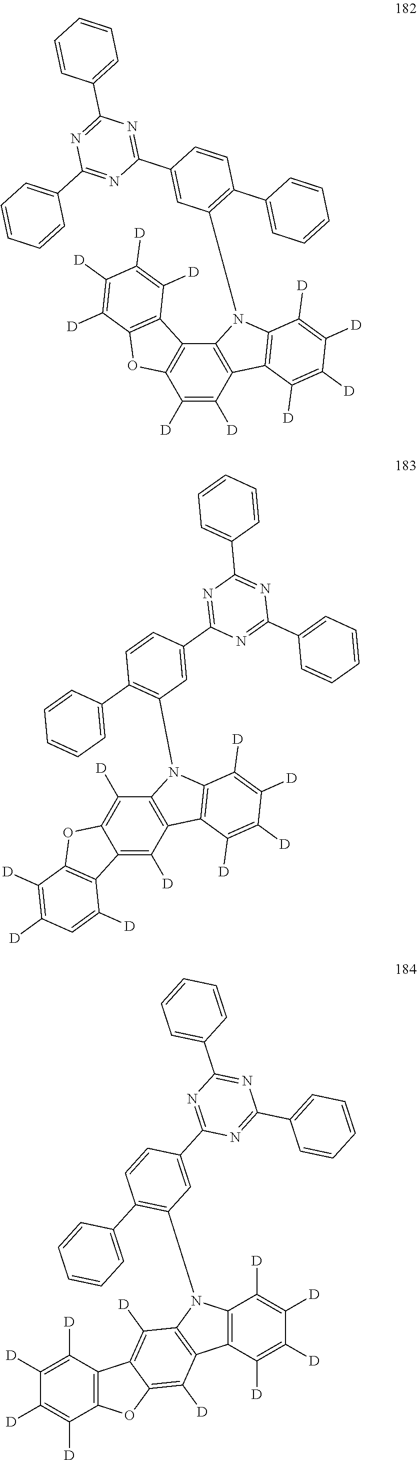

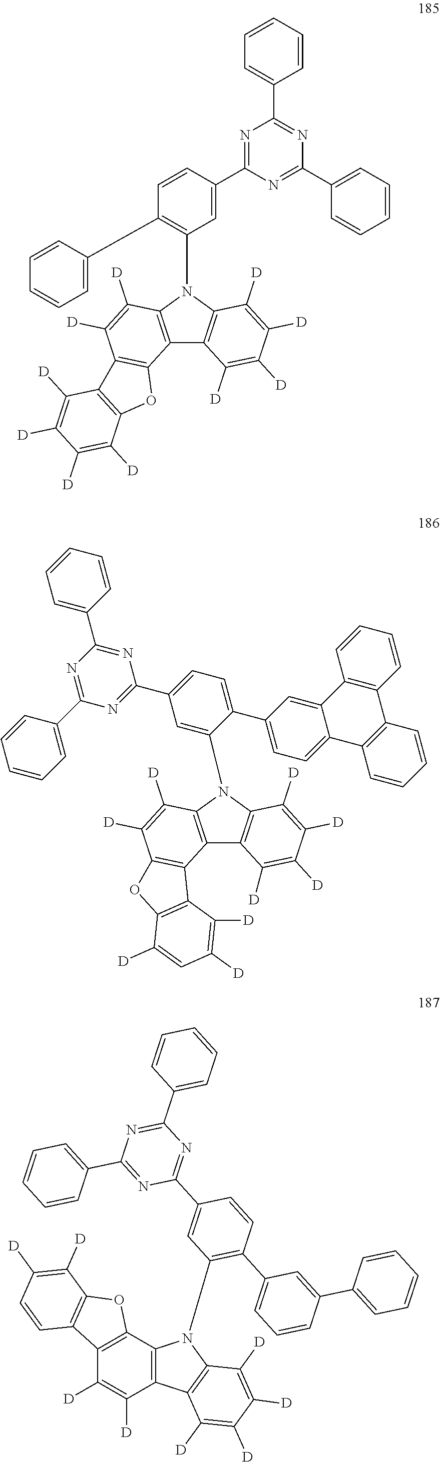 [SMILES: [2H]c1c([2H])c([2H])c2c(oc3c2c([2H])c([2H])c2c3c3c([2H])c([2H])c([2H])c([2H])c3n2-c2cc(-c3nc(-c4ccccc4)nc(-c4ccccc4)n3)ccc2-c2ccccc2)c1[2H] ; [2H]c1cc([2H])c2oc3c([2H])c([2H])c4c(c5c([2H])c([2H])c([2H])c([2H])c5n4-c4cc(-c5nc(-c6ccccc6)nc(-c6ccccc6)n5)ccc4-c4ccc5c6ccccc6c6ccccc6c5c4)c3c2c1[2H] ; [2H]c1ccc2c(oc3c2c([2H])c([2H])c2c4c([2H])c([2H])c([2H])c([2H])c4n(-c4cc(-c5nc(-c6ccccc6)nc(-c6ccccc6)n5)ccc4-c4cccc(-c5ccccc5)c4)c32)c1[2H]]

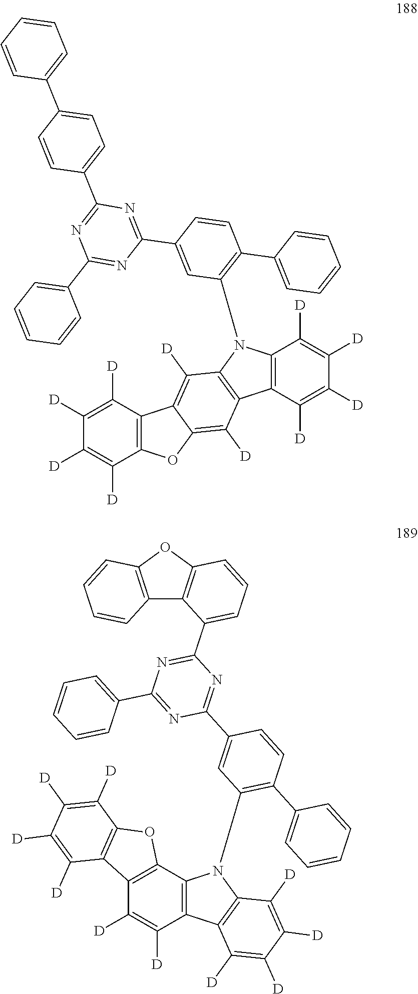 [SMILES: [2H]c1c([2H])c([2H])c2c(oc3c([2H])c4c5c([2H])c([2H])c([2H])c([2H])c5n(-c5cc(-c6nc(-c7ccccc7)nc(-c7ccc(-c8ccccc8)cc7)n6)ccc5-c5ccccc5)c4c([2H])c32)c1[2H] ; [2H]c1c([2H])c([2H])c2c(oc3c2c([2H])c([2H])c2c4c([2H])c([2H])c([2H])c([2H])c4n(-c4cc(-c5nc(-c6ccccc6)nc(-c6cccc7oc8ccccc8c67)n5)ccc4-c4ccccc4)c32)c1[2H]]

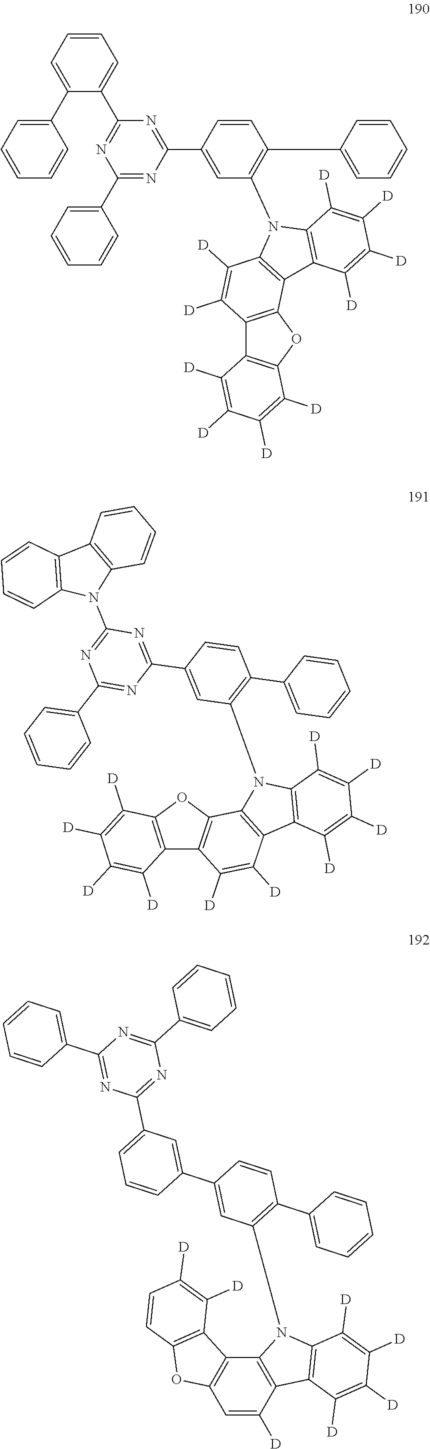 [SMILES: [2H]c1c([2H])c([2H])c2c(oc3c2c([2H])c([2H])c2c3c3c([2H])c([2H])c([2H])c([2H])c3n2-c2cc(-c3nc(-c4ccccc4)nc(-c4ccccc4-c4ccccc4)n3)ccc2-c2ccccc2)c1[2H] ; [2H]c1c([2H])c([2H])c2c(oc3c2c([2H])c([2H])c2c4c([2H])c([2H])c([2H])c([2H])c4n(-c4cc(-c5nc(-c6ccccc6)nc(-n6c7ccccc7c7ccccc76)n5)ccc4-c4ccccc4)c32)c1[2H] ; [2H]c1ccc2oc3cc([2H])c4c5c([2H])c([2H])c([2H])c([2H])c5n(-c5cc(-c6cccc(-c7nc(-c8ccccc8)nc(-c8ccccc8)n7)c6)ccc5-c5ccccc5)c4c3c2c1[2H]]

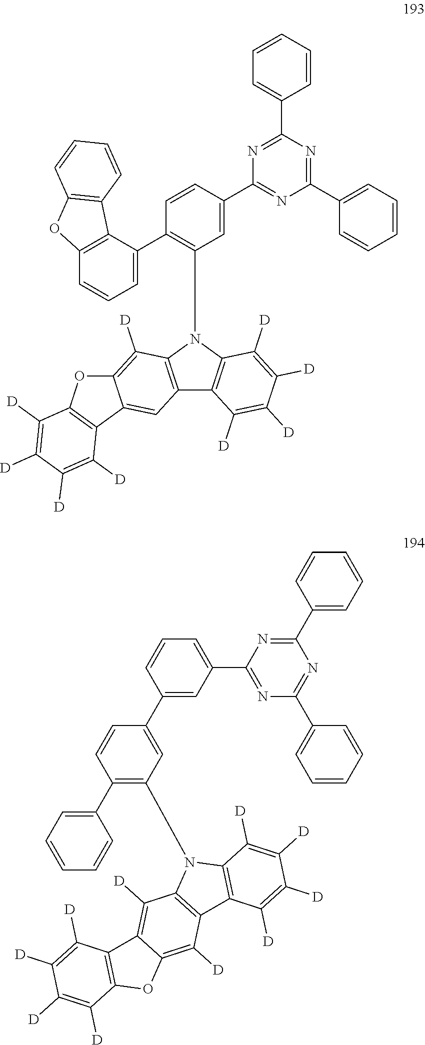 [SMILES: [2H]c1c([2H])c([2H])c2c(oc3c([2H])c4c(cc32)c2c([2H])c([2H])c([2H])c([2H])c2n4-c2cc(-c3nc(-c4ccccc4)nc(-c4ccccc4)n3)ccc2-c2cccc3oc4ccccc4c23)c1[2H] ; [2H]c1c([2H])c([2H])c2c(oc3c([2H])c4c5c([2H])c([2H])c([2H])c([2H])c5n(-c5cc(-c6cccc(-c7nc(-c8ccccc8)nc(-c8ccccc8)n7)c6)ccc5-c5ccccc5)c4c([2H])c32)c1[2H]]

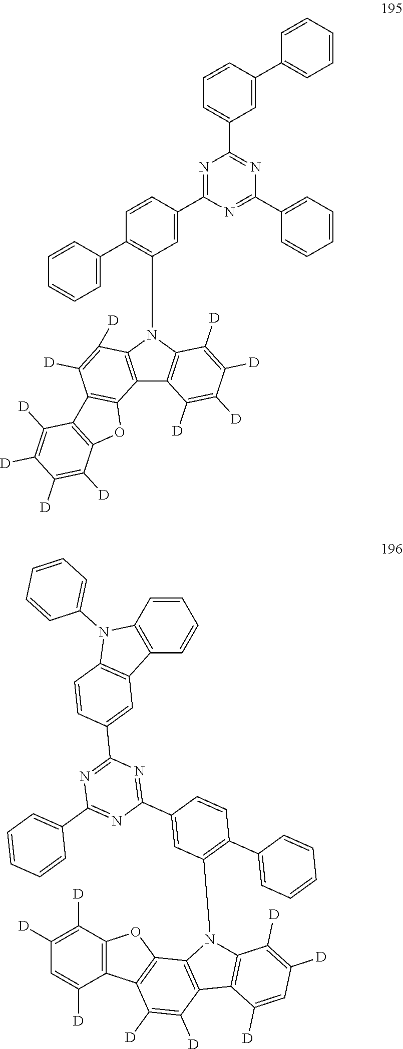 [SMILES: [2H]c1c([2H])c([2H])c2c(oc3c2c([2H])c([2H])c2c3c3c([2H])c([2H])c([2H])c([2H])c3n2-c2cc(-c3nc(-c4ccccc4)nc(-c4cccc(-c5ccccc5)c4)n3)ccc2-c2ccccc2)c1[2H] ; [2H]c1cc([2H])c2c(oc3c2c([2H])c([2H])c2c4c([2H])cc([2H])c([2H])c4n(-c4cc(-c5nc(-c6ccccc6)nc(-c6ccc7c(c6)c6ccccc6n7-c6ccccc6)n5)ccc4-c4ccccc4)c32)c1[2H]]

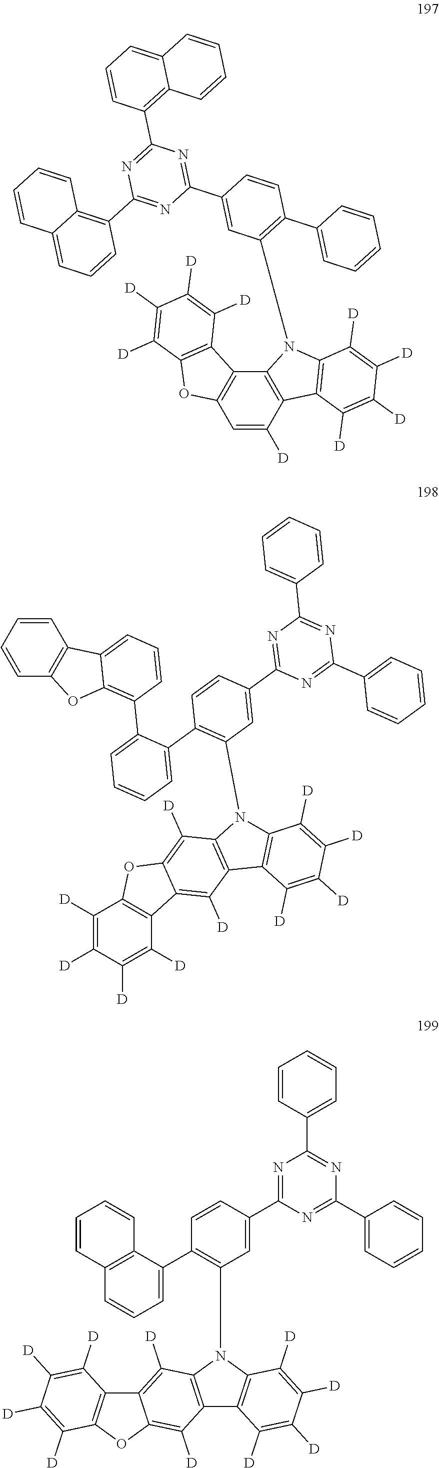 [SMILES: [2H]c1c([2H])c([2H])c2c(oc3cc([2H])c4c5c([2H])c([2H])c([2H])c([2H])c5n(-c5cc(-c6nc(-c7cccc8ccccc78)nc(-c7cccc8ccccc78)n6)ccc5-c5ccccc5)c4c32)c1[2H] ; [2H]c1c([2H])c([2H])c2c(oc3c([2H])c4c(c([2H])c32)c2c([2H])c([2H])c([2H])c([2H])c2n4-c2cc(-c3nc(-c4ccccc4)nc(-c4ccccc4)n3)ccc2-c2ccccc2-c2cccc3c2oc2ccccc23)c1[2H] ; [2H]c1c([2H])c([2H])c2c(oc3c([2H])c4c5c([2H])c([2H])c([2H])c([2H])c5n(-c5cc(-c6nc(-c7ccccc7)nc(-c7ccccc7)n6)ccc5-c5cccc6ccccc56)c4c([2H])c32)c1[2H]]

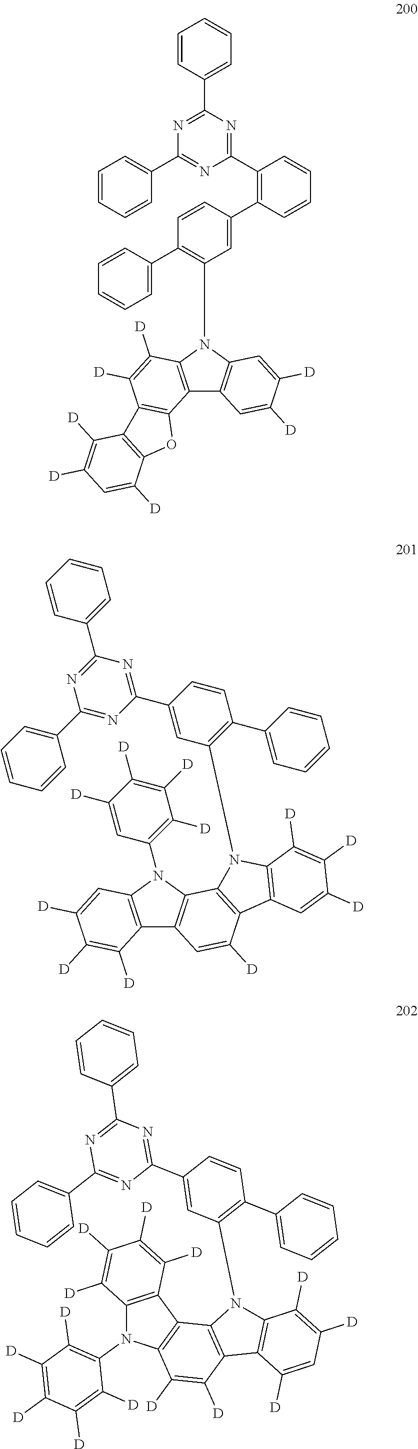

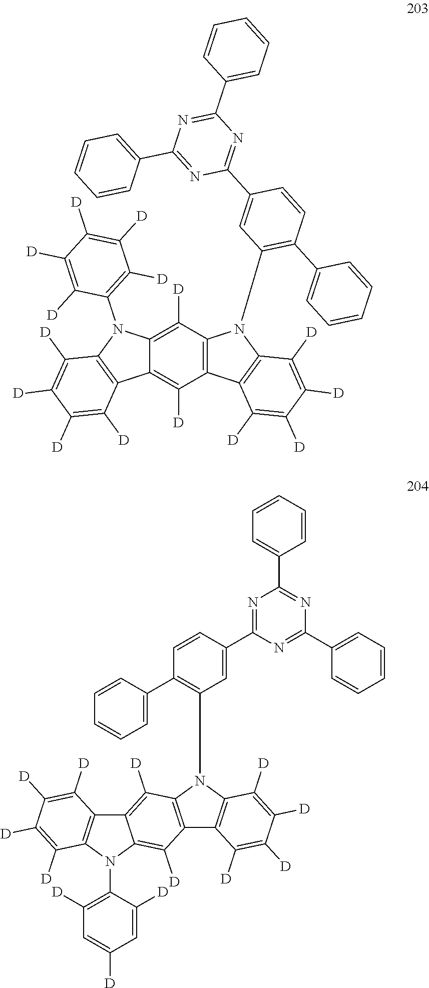 [SMILES: [2H]c1c([2H])c([2H])c(-n2c3c([2H])c([2H])c([2H])c([2H])c3c3c([2H])c4c5c([2H])c([2H])c([2H])c([2H])c5n(-c5cc(-c6nc(-c7ccccc7)nc(-c7ccccc7)n6)ccc5-c5ccccc5)c4c([2H])c32)c([2H])c1[2H] ; [2H]c1cc([2H])c(-n2c3c([2H])c([2H])c([2H])c([2H])c3c3c([2H])c4c(c([2H])c32)c2c([2H])c([2H])c([2H])c([2H])c2n4-c2cc(-c3nc(-c4ccccc4)nc(-c4ccccc4)n3)ccc2-c2ccccc2)c([2H])c1]

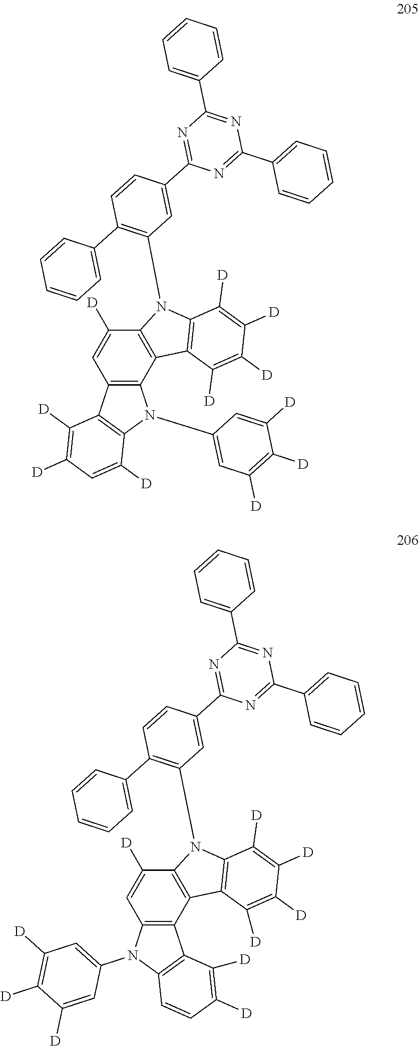 [SMILES: [2H]c1cc(-n2c3c([2H])cc([2H])c([2H])c3c3cc([2H])c4c(c5c([2H])c([2H])c([2H])c([2H])c5n4-c4cc(-c5nc(-c6ccccc6)nc(-c6ccccc6)n5)ccc4-c4ccccc4)c32)cc([2H])c1[2H] ; [2H]c1cc(-n2c3ccc([2H])c([2H])c3c3c4c5c([2H])c([2H])c([2H])c([2H])c5n(-c5cc(-c6nc(-c7ccccc7)nc(-c7ccccc7)n6)ccc5-c5ccccc5)c4c([2H])cc32)cc([2H])c1[2H]]

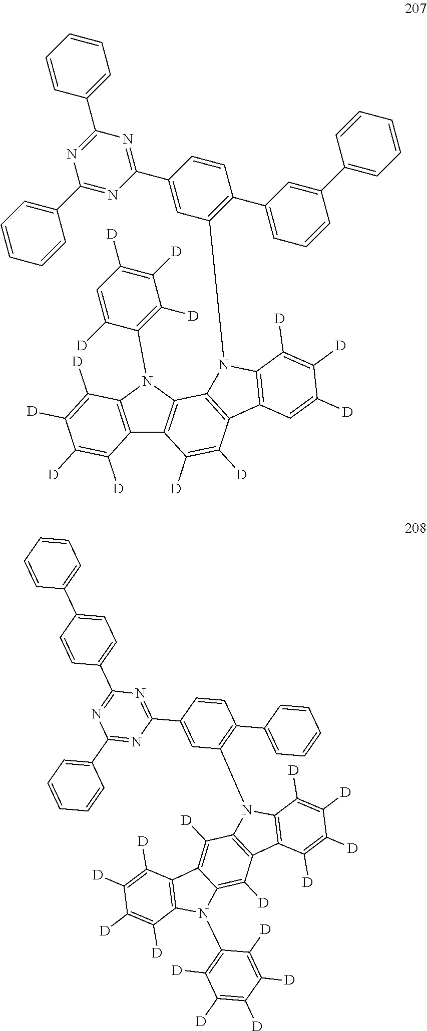 [SMILES: [2H]c1cc([2H])c(-n2c3c([2H])c([2H])c([2H])c([2H])c3c3c([2H])c([2H])c4c5cc([2H])c([2H])c([2H])c5n(-c5cc(-c6nc(-c7ccccc7)nc(-c7ccccc7)n6)ccc5-c5cccc(-c6ccccc6)c5)c4c32)c([2H])c1[2H] ; [2H]c1c([2H])c([2H])c(-n2c3c([2H])c([2H])c([2H])c([2H])c3c3c([2H])c4c(c([2H])c32)c2c([2H])c([2H])c([2H])c([2H])c2n4-c2cc(-c3nc(-c4ccccc4)nc(-c4ccc(-c5ccccc5)cc4)n3)ccc2-c2ccccc2)c([2H])c1[2H]]

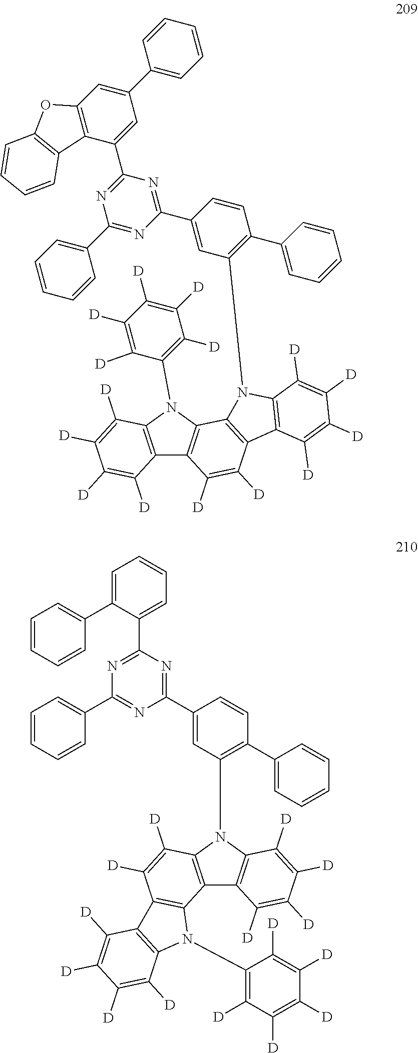 [SMILES: [2H]c1c([2H])c([2H])c(-n2c3c([2H])c([2H])c([2H])c([2H])c3c3c([2H])c([2H])c4c5c([2H])c([2H])c([2H])c([2H])c5n(-c5cc(-c6nc(-c7ccccc7)nc(-c7cc(-c8ccccc8)cc8oc9ccccc9c78)n6)ccc5-c5ccccc5)c4c32)c([2H])c1[2H] ; [2H]c1c([2H])c([2H])c(-n2c3c([2H])c([2H])c([2H])c([2H])c3c3c([2H])c([2H])c4c(c5c([2H])c([2H])c([2H])c([2H])c5n4-c4cc(-c5nc(-c6ccccc6)nc(-c6ccccc6-c6ccccc6)n5)ccc4-c4ccccc4)c32)c([2H])c1[2H]]

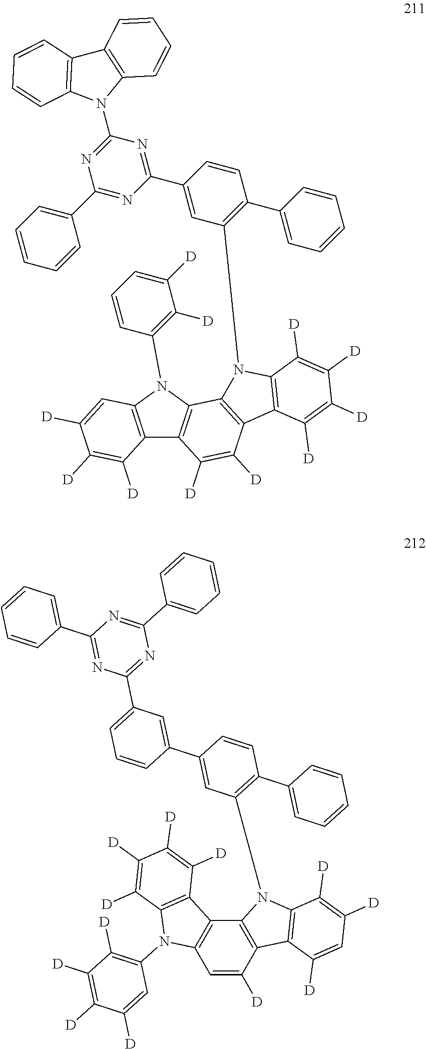 [SMILES: [2H]c1cccc(-n2c3cc([2H])c([2H])c([2H])c3c3c([2H])c([2H])c4c5c([2H])c([2H])c([2H])c([2H])c5n(-c5cc(-c6nc(-c7ccccc7)nc(-n7c8ccccc8c8ccccc87)n6)ccc5-c5ccccc5)c4c32)c1[2H] ; [2H]c1cc(-n2c3cc([2H])c4c5c([2H])cc([2H])c([2H])c5n(-c5cc(-c6cccc(-c7nc(-c8ccccc8)nc(-c8ccccc8)n7)c6)ccc5-c5ccccc5)c4c3c3c([2H])c([2H])c([2H])c([2H])c32)c([2H])c([2H])c1[2H]]

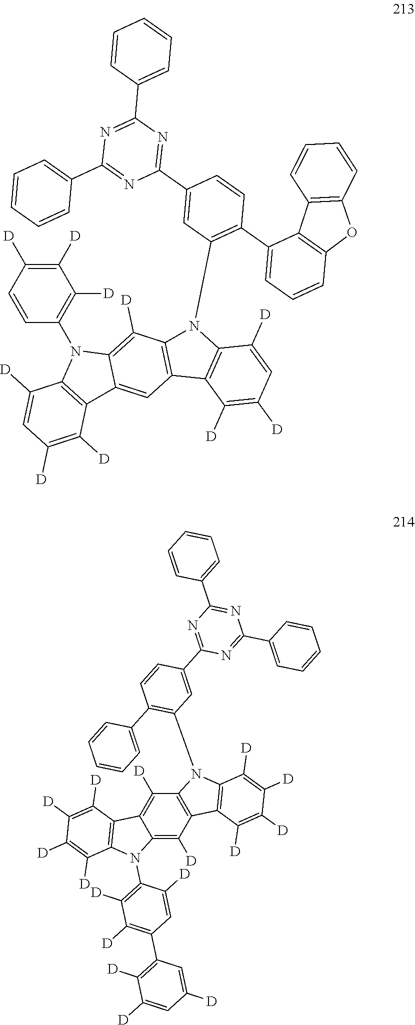 [SMILES: [2H]c1ccc(-n2c3c([2H])c4c(cc3c3c([2H])c([2H])cc([2H])c32)c2c([2H])c([2H])cc([2H])c2n4-c2cc(-c3nc(-c4ccccc4)nc(-c4ccccc4)n3)ccc2-c2cccc3oc4ccccc4c23)c([2H])c1[2H] ; [2H]c1cc([2H])c([2H])c(-c2cc([2H])c(-n3c4c([2H])c([2H])c([2H])c([2H])c4c4c([2H])c5c(c([2H])c43)c3c([2H])c([2H])c([2H])c([2H])c3n5-c3cc(-c4nc(-c5ccccc5)nc(-c5ccccc5)n4)ccc3-c3ccccc3)c([2H])c2[2H])c1]

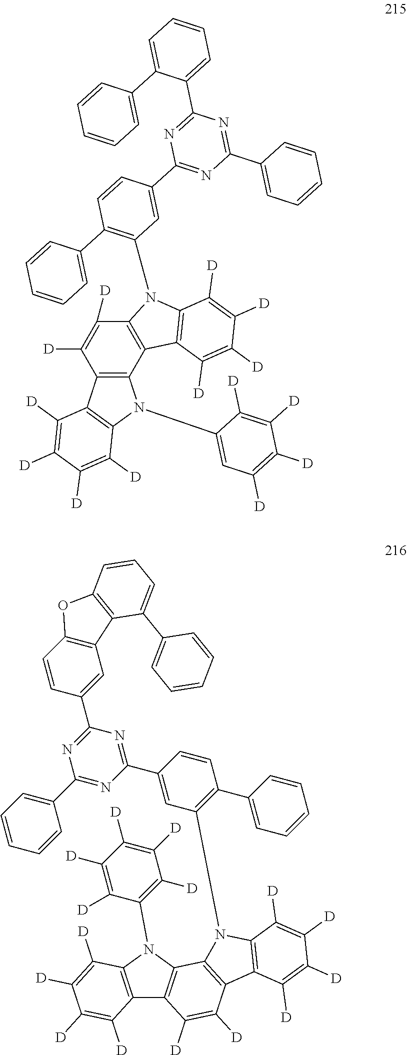 [SMILES: [2H]c1cc(-n2c3c([2H])c([2H])c([2H])c([2H])c3c3c([2H])c([2H])c4c(c5c([2H])c([2H])c([2H])c([2H])c5n4-c4cc(-c5nc(-c6ccccc6)nc(-c6ccccc6-c6ccccc6)n5)ccc4-c4ccccc4)c32)c([2H])c([2H])c1[2H] ; [2H]c1c([2H])c([2H])c(-n2c3c([2H])c([2H])c([2H])c([2H])c3c3c([2H])c([2H])c4c5c([2H])c([2H])c([2H])c([2H])c5n(-c5cc(-c6nc(-c7ccccc7)nc(-c7ccc8oc9cccc(-c%10ccccc%10)c9c8c7)n6)ccc5-c5ccccc5)c4c32)c([2H])c1[2H]]

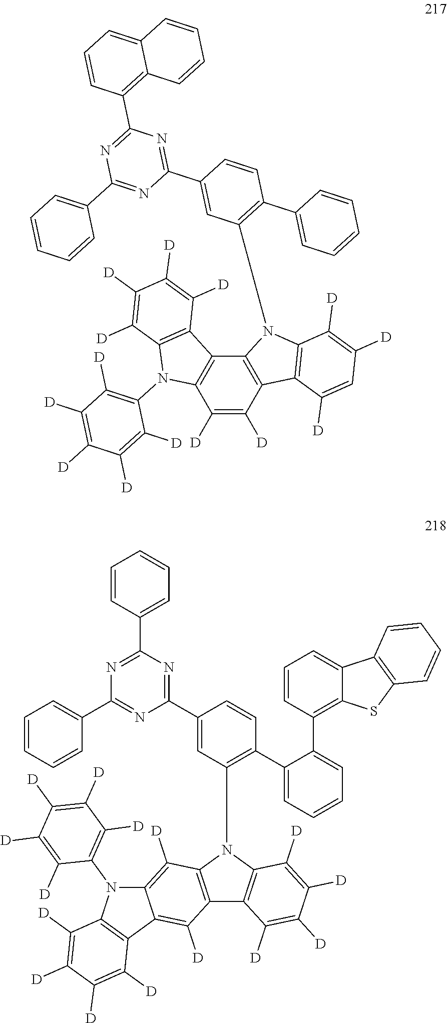 [SMILES: [2H]c1cc([2H])c2c3c([2H])c([2H])c4c(c5c([2H])c([2H])c([2H])c([2H])c5n4-c4c([2H])c([2H])c([2H])c([2H])c4[2H])c3n(-c3cc(-c4nc(-c5ccccc5)nc(-c5cccc6ccccc56)n4)ccc3-c3ccccc3)c2c1[2H] ; [2H]c1c([2H])c([2H])c(-n2c3c([2H])c([2H])c([2H])c([2H])c3c3c([2H])c4c5c([2H])c([2H])c([2H])c([2H])c5n(-c5cc(-c6nc(-c7ccccc7)nc(-c7ccccc7)n6)ccc5-c5ccccc5-c5cccc6c5sc5ccccc56)c4c([2H])c32)c([2H])c1[2H]]

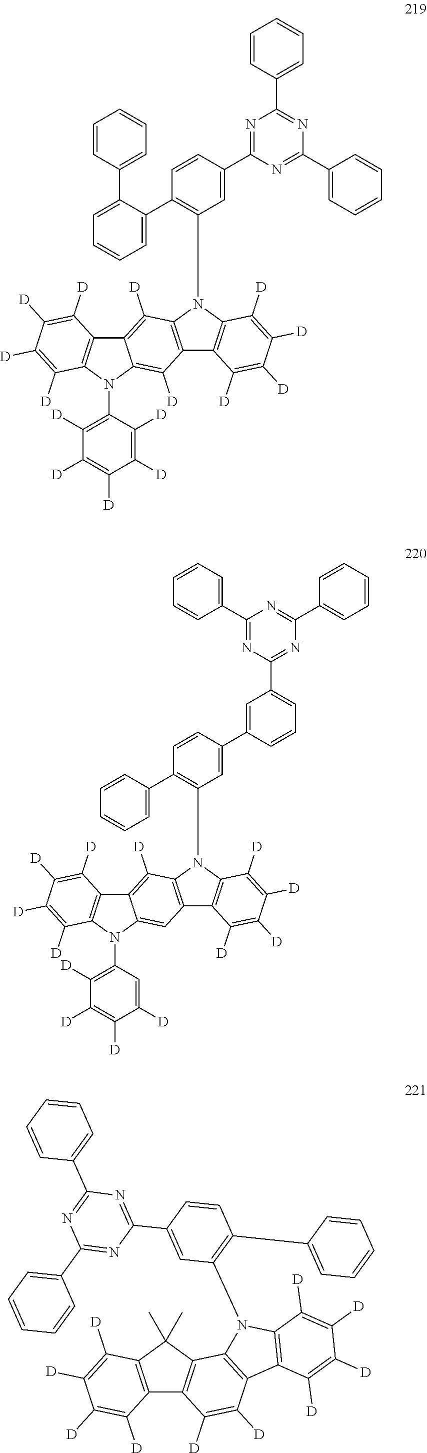

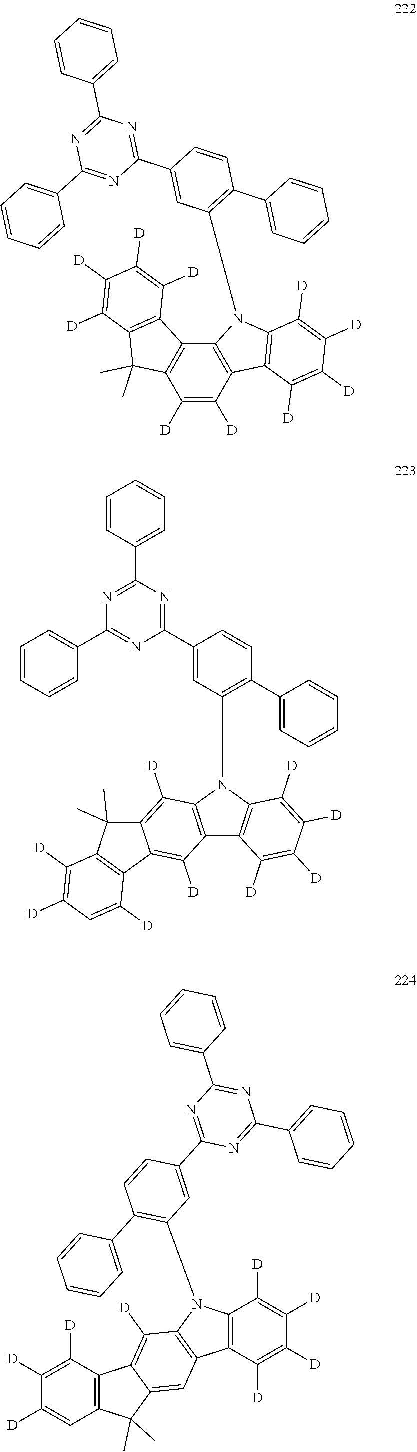 [SMILES: [2H]c1c([2H])c([2H])c2c(c1[2H])-c1c(c([2H])c([2H])c3c4c([2H])c([2H])c([2H])c([2H])c4n(-c4cc(-c5nc(-c6ccccc6)nc(-c6ccccc6)n5)ccc4-c4ccccc4)c13)C2(C)C ; [2H]c1cc([2H])c2c(c1[2H])C(C)(C)c1c-2c([2H])c2c3c([2H])c([2H])c([2H])c([2H])c3n(-c3cc(-c4nc(-c5ccccc5)nc(-c5ccccc5)n4)ccc3-c3ccccc3)c2c1[2H] ; [2H]c1cc2c(c([2H])c1[2H])-c1c(cc3c4c([2H])c([2H])c([2H])c([2H])c4n(-c4cc(-c5nc(-c6ccccc6)nc(-c6ccccc6)n5)ccc4-c4ccccc4)c3c1[2H])C2(C)C]

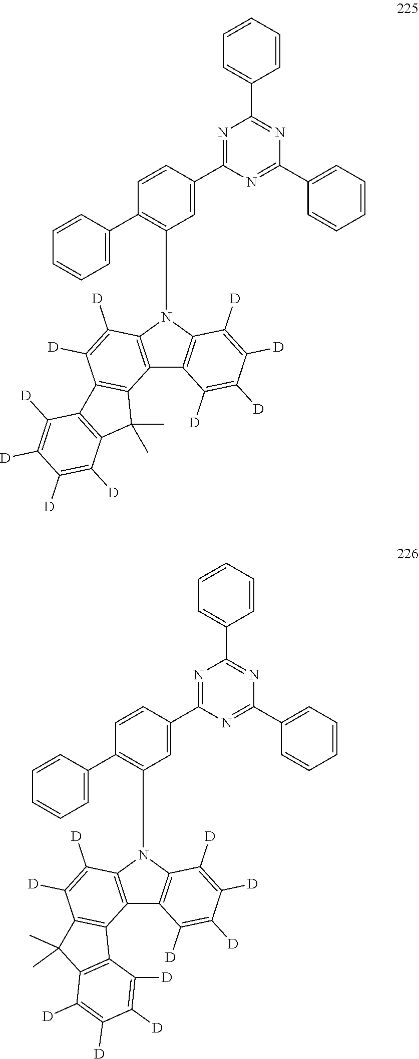 [SMILES: [2H]c1c([2H])c([2H])c2c(c1[2H])-c1c([2H])c([2H])c3c(c1C2(C)C)c1c([2H])c([2H])c([2H])c([2H])c1n3-c1cc(-c2nc(-c3ccccc3)nc(-c3ccccc3)n2)ccc1-c1ccccc1 ; [2H]c1c([2H])c([2H])c2c(c1[2H])-c1c(c([2H])c([2H])c3c1c1c([2H])c([2H])c([2H])c([2H])c1n3-c1cc(-c3nc(-c4ccccc4)nc(-c4ccccc4)n3)ccc1-c1ccccc1)C2(C)C]

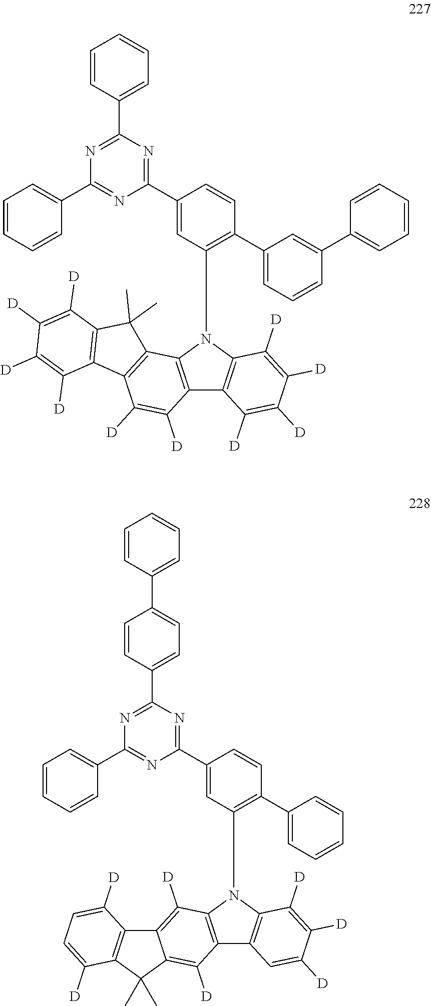 [SMILES: [2H]c1c([2H])c([2H])c2c(c1[2H])-c1c([2H])c([2H])c3c4c([2H])c([2H])c([2H])c([2H])c4n(-c4cc(-c5nc(-c6ccccc6)nc(-c6ccccc6)n5)ccc4-c4cccc(-c5ccccc5)c4)c3c1C2(C)C ; [2H]c1cc2c3c([2H])c4c(c([2H])c3n(-c3cc(-c5nc(-c6ccccc6)nc(-c6ccc(-c7ccccc7)cc6)n5)ccc3-c3ccccc3)c2c([2H])c1[2H])-c1c([2H])ccc([2H])c1C4(C)C]

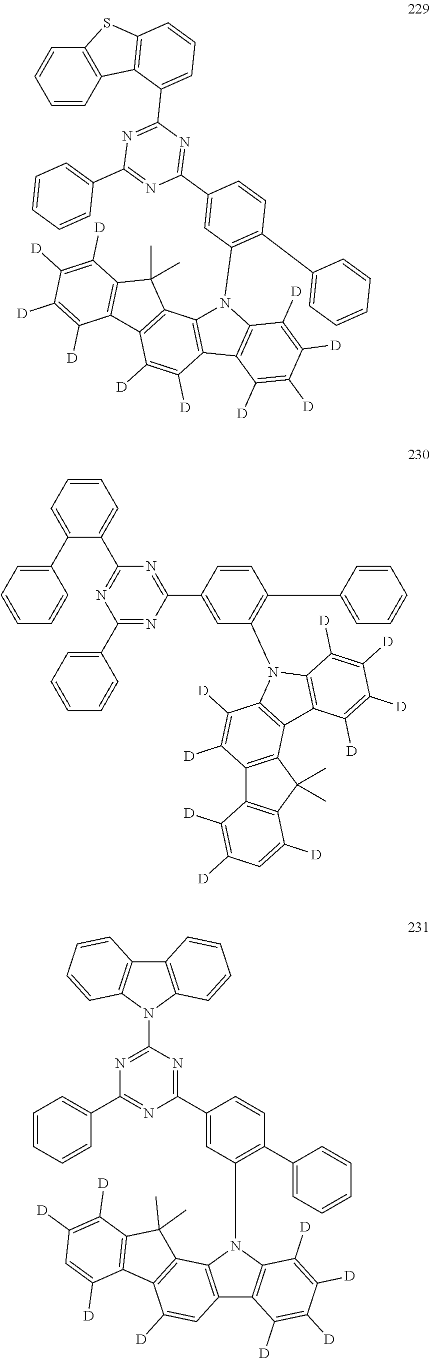 [SMILES: [2H]c1c([2H])c([2H])c2c(c1[2H])-c1c([2H])c([2H])c3c4c([2H])c([2H])c([2H])c([2H])c4n(-c4cc(-c5nc(-c6ccccc6)nc(-c6cccc7sc8ccccc8c67)n5)ccc4-c4ccccc4)c3c1C2(C)C ; [2H]c1cc([2H])c2c(c1[2H])-c1c([2H])c([2H])c3c(c1C2(C)C)c1c([2H])c([2H])c([2H])c([2H])c1n3-c1cc(-c2nc(-c3ccccc3)nc(-c3ccccc3-c3ccccc3)n2)ccc1-c1ccccc1 ; [2H]c1cc([2H])c2c(c1[2H])C(C)(C)c1c-2c([2H])cc2c3c([2H])c([2H])c([2H])c([2H])c3n(-c3cc(-c4nc(-c5ccccc5)nc(-n5c6ccccc6c6ccccc65)n4)ccc3-c3ccccc3)c12]

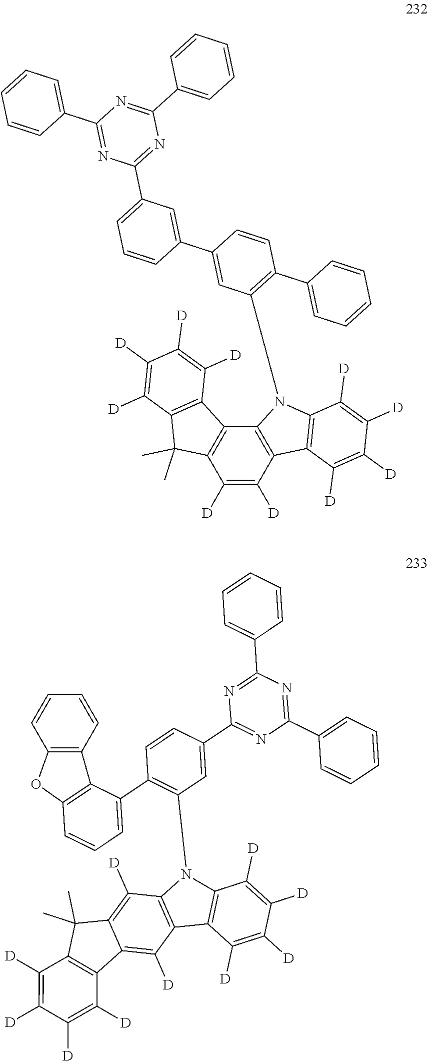 [SMILES: [2H]c1c([2H])c([2H])c2c(c1[2H])-c1c(c([2H])c([2H])c3c4c([2H])c([2H])c([2H])c([2H])c4n(-c4cc(-c5cccc(-c6nc(-c7ccccc7)nc(-c7ccccc7)n6)c5)ccc4-c4ccccc4)c13)C2(C)C ; [2H]c1c([2H])c([2H])c2c(c1[2H])-c1c(c([2H])c3c(c1[2H])c1c([2H])c([2H])c([2H])c([2H])c1n3-c1cc(-c3nc(-c4ccccc4)nc(-c4ccccc4)n3)ccc1-c1cccc3oc4ccccc4c13)C2(C)C]

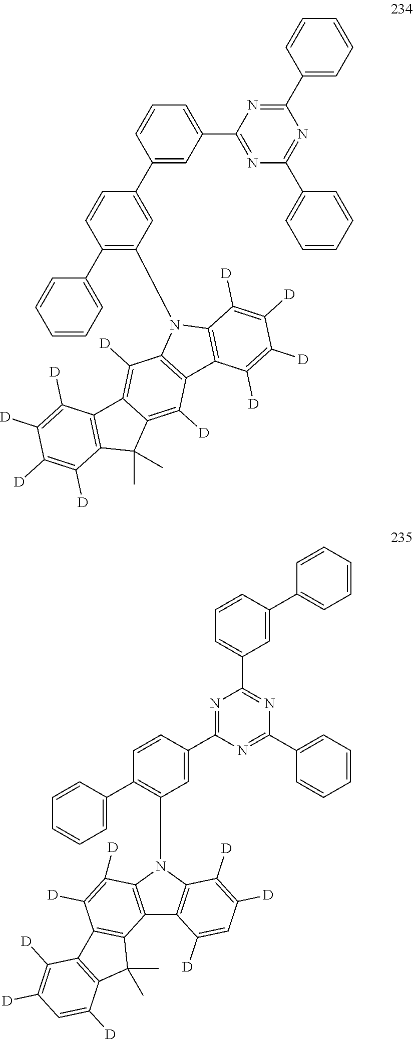 [SMILES: [2H]c1c([2H])c([2H])c2c(c1[2H])-c1c(c([2H])c3c4c([2H])c([2H])c([2H])c([2H])c4n(-c4cc(-c5cccc(-c6nc(-c7ccccc7)nc(-c7ccccc7)n6)c5)ccc4-c4ccccc4)c3c1[2H])C2(C)C ; [2H]c1cc([2H])c2c(c1[2H])-c1c([2H])c([2H])c3c(c1C2(C)C)c1c([2H])cc([2H])c([2H])c1n3-c1cc(-c2nc(-c3ccccc3)nc(-c3cccc(-c4ccccc4)c3)n2)ccc1-c1ccccc1]

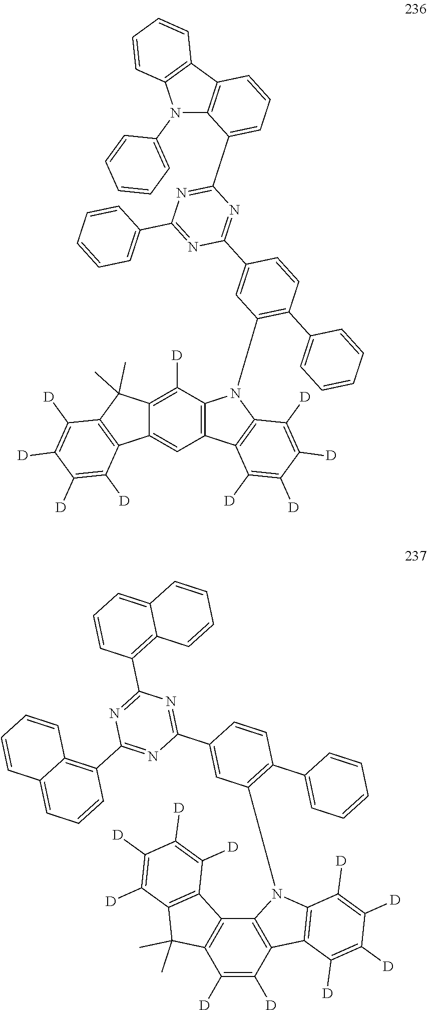 [SMILES: [2H]c1c([2H])c([2H])c2c(c1[2H])-c1cc3c4c([2H])c([2H])c([2H])c([2H])c4n(-c4cc(-c5nc(-c6ccccc6)nc(-c6cccc7c8ccccc8n(-c8ccccc8)c67)n5)ccc4-c4ccccc4)c3c([2H])c1C2(C)C ; [2H]c1c([2H])c([2H])c2c(c1[2H])-c1c(c([2H])c([2H])c3c4c([2H])c([2H])c([2H])c([2H])c4n(-c4cc(-c5nc(-c6cccc7ccccc67)nc(-c6cccc7ccccc67)n5)ccc4-c4ccccc4)c13)C2(C)C]

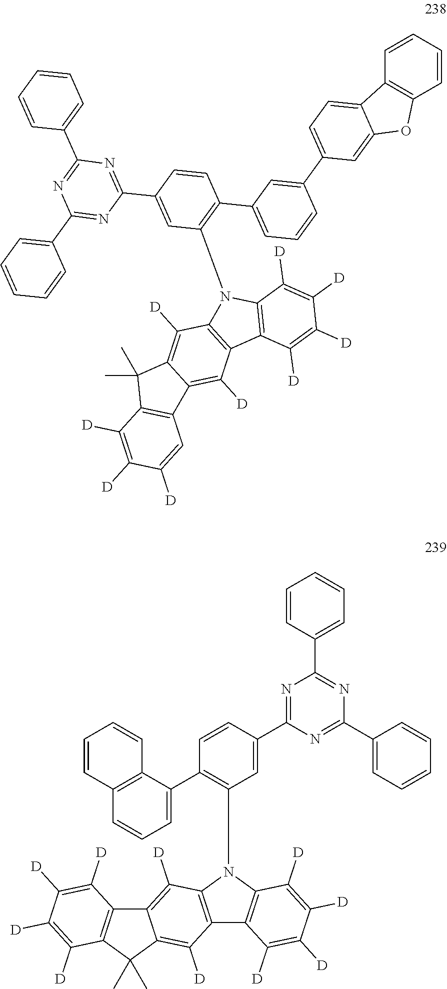 [SMILES: [2H]c1cc2c(c([2H])c1[2H])C(C)(C)c1c-2c([2H])c2c3c([2H])c([2H])c([2H])c([2H])c3n(-c3cc(-c4nc(-c5ccccc5)nc(-c5ccccc5)n4)ccc3-c3cccc(-c4ccc5c(c4)oc4ccccc45)c3)c2c1[2H] ; [2H]c1c([2H])c([2H])c2c(c1[2H])-c1c(c([2H])c3c4c([2H])c([2H])c([2H])c([2H])c4n(-c4cc(-c5nc(-c6ccccc6)nc(-c6ccccc6)n5)ccc4-c4cccc5ccccc45)c3c1[2H])C2(C)C]

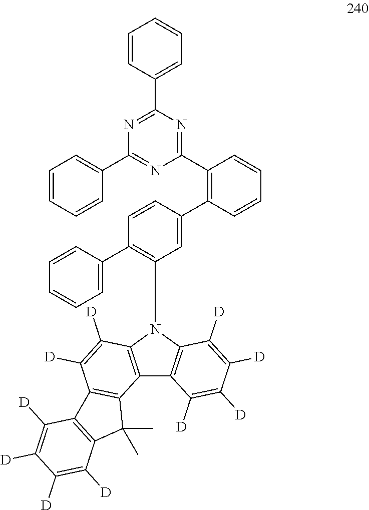 [SMILES: [2H]c1c([2H])c([2H])c2c(c1[2H])-c1c([2H])c([2H])c3c(c1C2(C)C)c1c([2H])c([2H])c([2H])c([2H])c1n3-c1cc(-c2ccccc2-c2nc(-c3ccccc3)nc(-c3ccccc3)n2)ccc1-c1ccccc1]

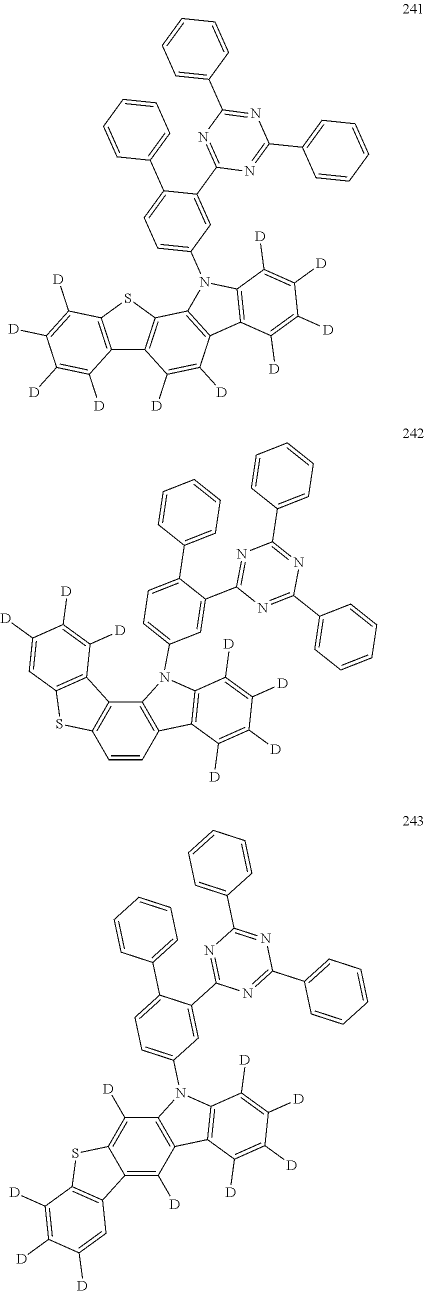 [SMILES: [2H]c1c([2H])c([2H])c2c(sc3c2c([2H])c([2H])c2c4c([2H])c([2H])c([2H])c([2H])c4n(-c4ccc(-c5ccccc5)c(-c5nc(-c6ccccc6)nc(-c6ccccc6)n5)c4)c32)c1[2H] ; [2H]c1cc2sc3ccc4c5c([2H])c([2H])c([2H])c([2H])c5n(-c5ccc(-c6ccccc6)c(-c6nc(-c7ccccc7)nc(-c7ccccc7)n6)c5)c4c3c2c([2H])c1[2H] ; [2H]c1cc2c(sc3c([2H])c4c(c([2H])c32)c2c([2H])c([2H])c([2H])c([2H])c2n4-c2ccc(-c3ccccc3)c(-c3nc(-c4ccccc4)nc(-c4ccccc4)n3)c2)c([2H])c1[2H]]

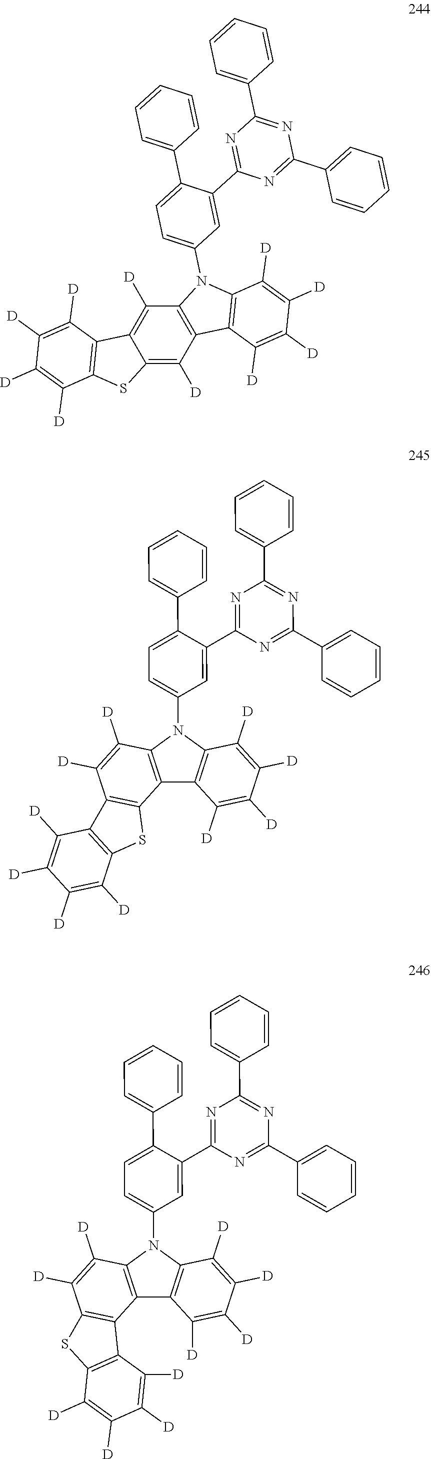 [SMILES: [2H]c1c([2H])c([2H])c2c(sc3c([2H])c4c5c([2H])c([2H])c([2H])c([2H])c5n(-c5ccc(-c6ccccc6)c(-c6nc(-c7ccccc7)nc(-c7ccccc7)n6)c5)c4c([2H])c32)c1[2H] ; [2H]c1c([2H])c([2H])c2c(sc3c2c([2H])c([2H])c2c3c3c([2H])c([2H])c([2H])c([2H])c3n2-c2ccc(-c3ccccc3)c(-c3nc(-c4ccccc4)nc(-c4ccccc4)n3)c2)c1[2H] ; [2H]c1c([2H])c([2H])c2c(sc3c([2H])c([2H])c4c(c5c([2H])c([2H])c([2H])c([2H])c5n4-c4ccc(-c5ccccc5)c(-c5nc(-c6ccccc6)nc(-c6ccccc6)n5)c4)c32)c1[2H]]

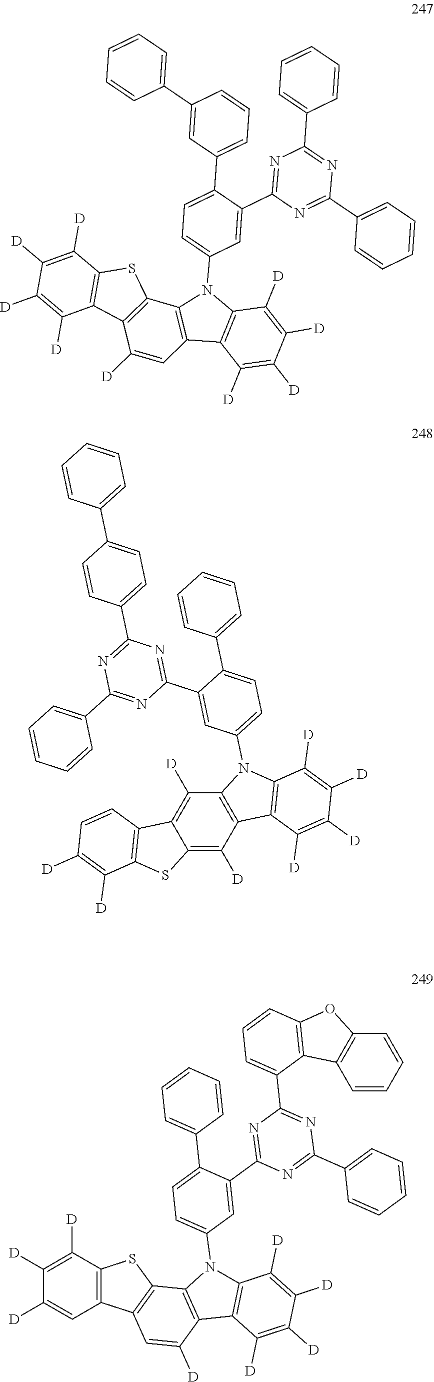 [SMILES: [2H]c1c([2H])c([2H])c2c(sc3c2c([2H])cc2c4c([2H])c([2H])c([2H])c([2H])c4n(-c4ccc(-c5cccc(-c6ccccc6)c5)c(-c5nc(-c6ccccc6)nc(-c6ccccc6)n5)c4)c23)c1[2H] ; [2H]c1ccc2c(sc3c([2H])c4c5c([2H])c([2H])c([2H])c([2H])c5n(-c5ccc(-c6ccccc6)c(-c6nc(-c7ccccc7)nc(-c7ccc(-c8ccccc8)cc7)n6)c5)c4c([2H])c32)c1[2H] ; [2H]c1cc2c(sc3c2cc([2H])c2c4c([2H])c([2H])c([2H])c([2H])c4n(-c4ccc(-c5ccccc5)c(-c5nc(-c6ccccc6)nc(-c6cccc7oc8ccccc8c67)n5)c4)c32)c([2H])c1[2H]]

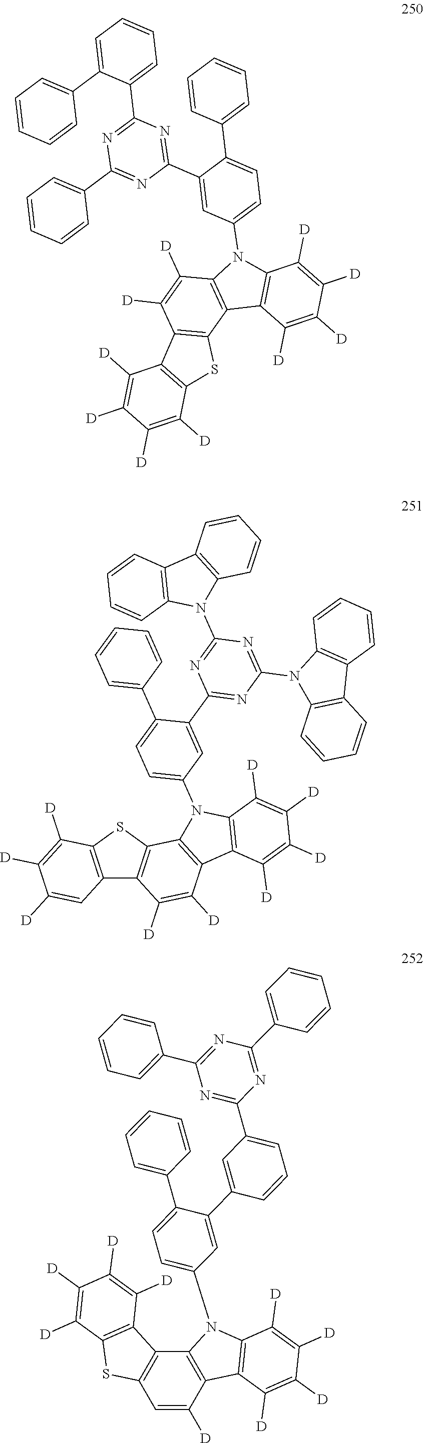 [SMILES: [2H]c1c([2H])c([2H])c2c(sc3c2c([2H])c([2H])c2c3c3c([2H])c([2H])c([2H])c([2H])c3n2-c2ccc(-c3ccccc3)c(-c3nc(-c4ccccc4)nc(-c4ccccc4-c4ccccc4)n3)c2)c1[2H] ; [2H]c1cc2c(sc3c2c([2H])c([2H])c2c4c([2H])c([2H])c([2H])c([2H])c4n(-c4ccc(-c5ccccc5)c(-c5nc(-n6c7ccccc7c7ccccc76)nc(-n6c7ccccc7c7ccccc76)n5)c4)c32)c([2H])c1[2H] ; [2H]c1c([2H])c([2H])c2c(sc3cc([2H])c4c5c([2H])c([2H])c([2H])c([2H])c5n(-c5ccc(-c6ccccc6)c(-c6cccc(-c7nc(-c8ccccc8)nc(-c8ccccc8)n7)c6)c5)c4c32)c1[2H]]

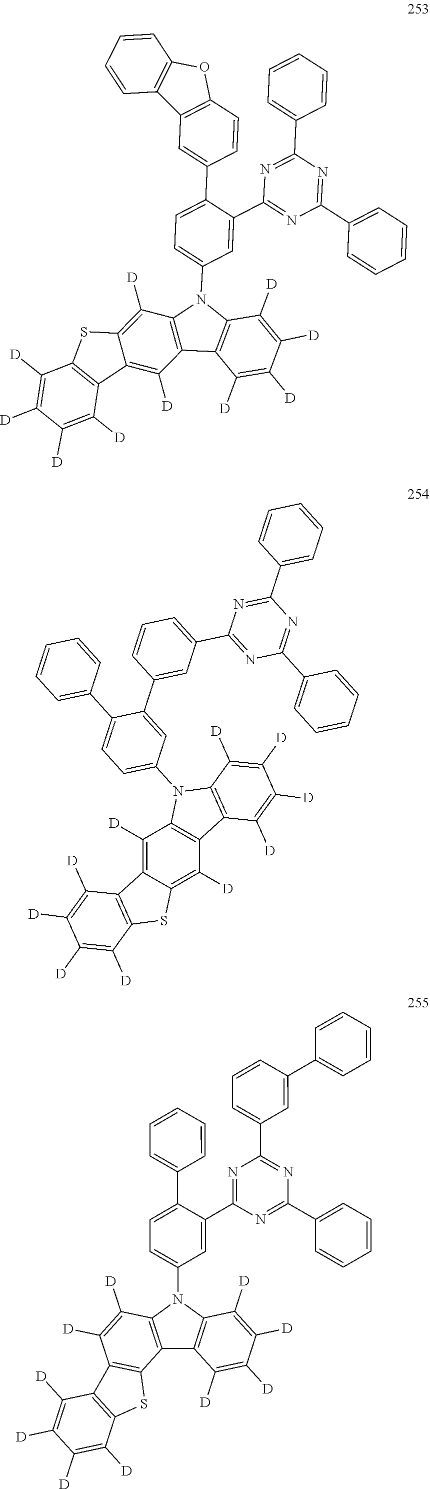 [SMILES: [2H]c1c([2H])c([2H])c2c(sc3c([2H])c4c(c([2H])c32)c2c([2H])c([2H])c([2H])c([2H])c2n4-c2ccc(-c3ccc4oc5ccccc5c4c3)c(-c3nc(-c4ccccc4)nc(-c4ccccc4)n3)c2)c1[2H] ; [2H]c1c([2H])c([2H])c2c(sc3c([2H])c4c5c([2H])c([2H])c([2H])c([2H])c5n(-c5ccc(-c6ccccc6)c(-c6cccc(-c7nc(-c8ccccc8)nc(-c8ccccc8)n7)c6)c5)c4c([2H])c32)c1[2H] ; [2H]c1c([2H])c([2H])c2c(sc3c2c([2H])c([2H])c2c3c3c([2H])c([2H])c([2H])c([2H])c3n2-c2ccc(-c3ccccc3)c(-c3nc(-c4ccccc4)nc(-c4cccc(-c5ccccc5)c4)n3)c2)c1[2H]]

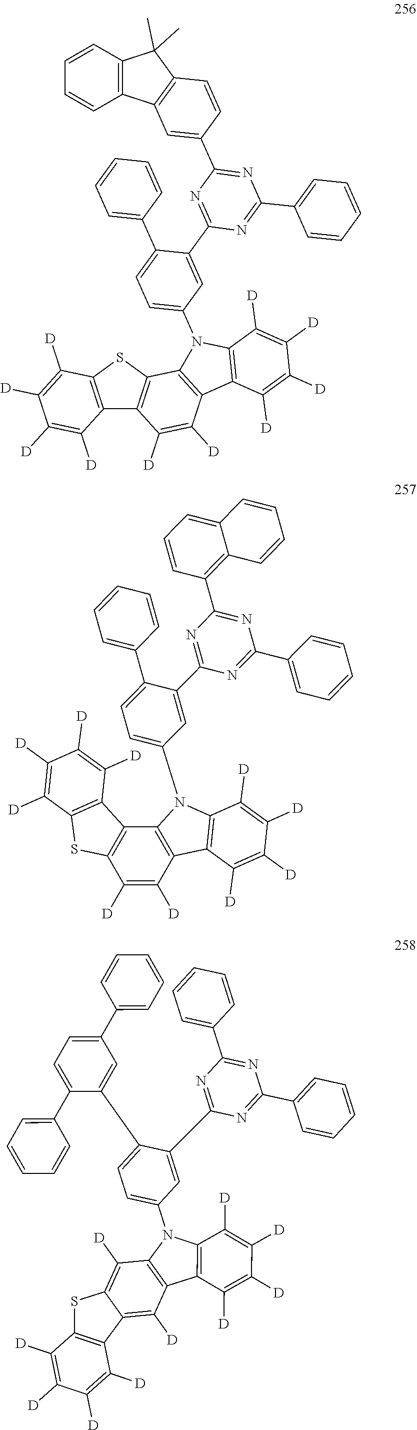

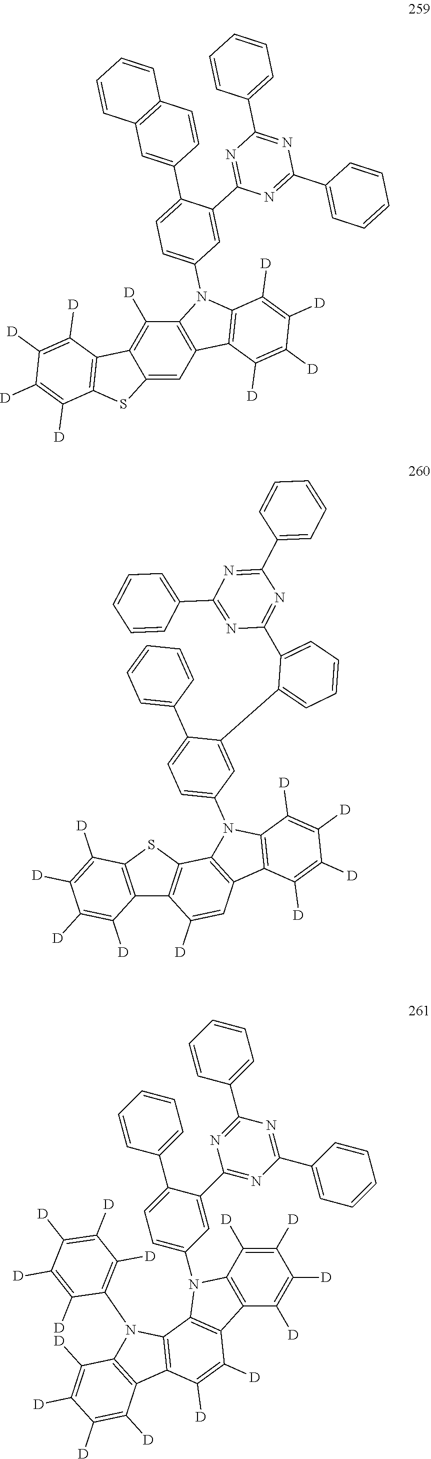 [SMILES: [2H]c1c([2H])c([2H])c2c(sc3cc4c5c([2H])c([2H])c([2H])c([2H])c5n(-c5ccc(-c6ccc7ccccc7c6)c(-c6nc(-c7ccccc7)nc(-c7ccccc7)n6)c5)c4c([2H])c32)c1[2H] ; [2H]c1c([2H])c([2H])c2c(sc3c2c([2H])cc2c4c([2H])c([2H])c([2H])c([2H])c4n(-c4ccc(-c5ccccc5)c(-c5ccccc5-c5nc(-c6ccccc6)nc(-c6ccccc6)n5)c4)c23)c1[2H] ; [2H]c1c([2H])c([2H])c(-n2c3c([2H])c([2H])c([2H])c([2H])c3c3c([2H])c([2H])c4c5c([2H])c([2H])c([2H])c([2H])c5n(-c5ccc(-c6ccccc6)c(-c6nc(-c7ccccc7)nc(-c7ccccc7)n6)c5)c4c32)c([2H])c1[2H]]

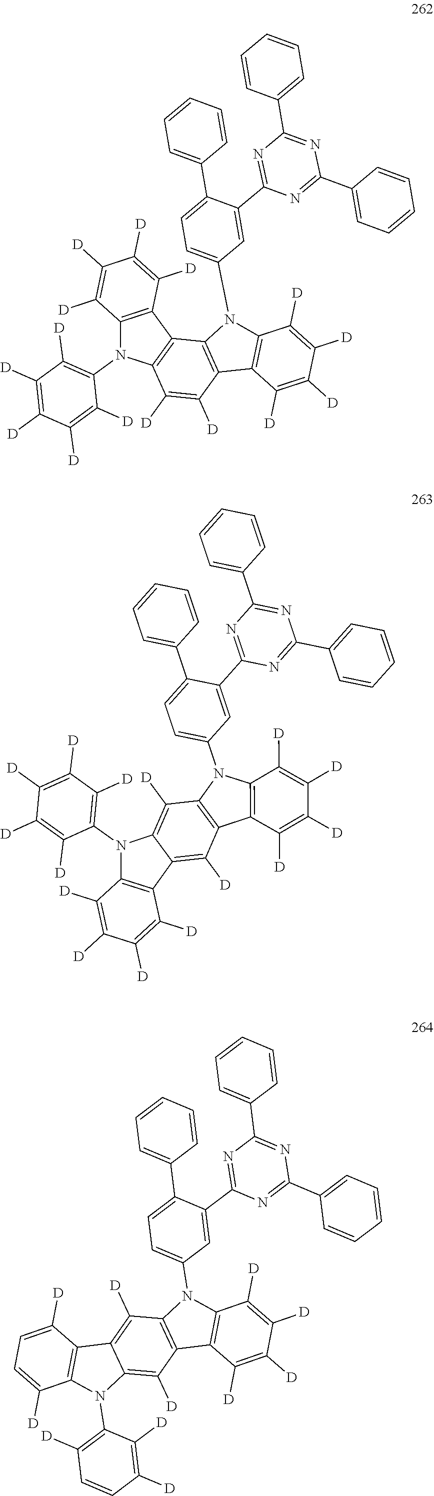

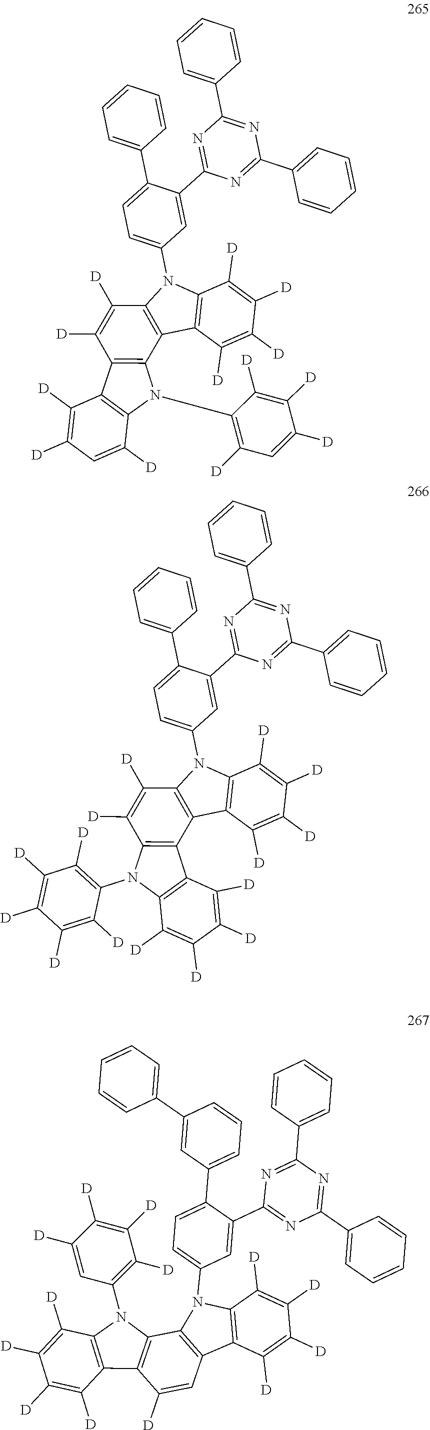

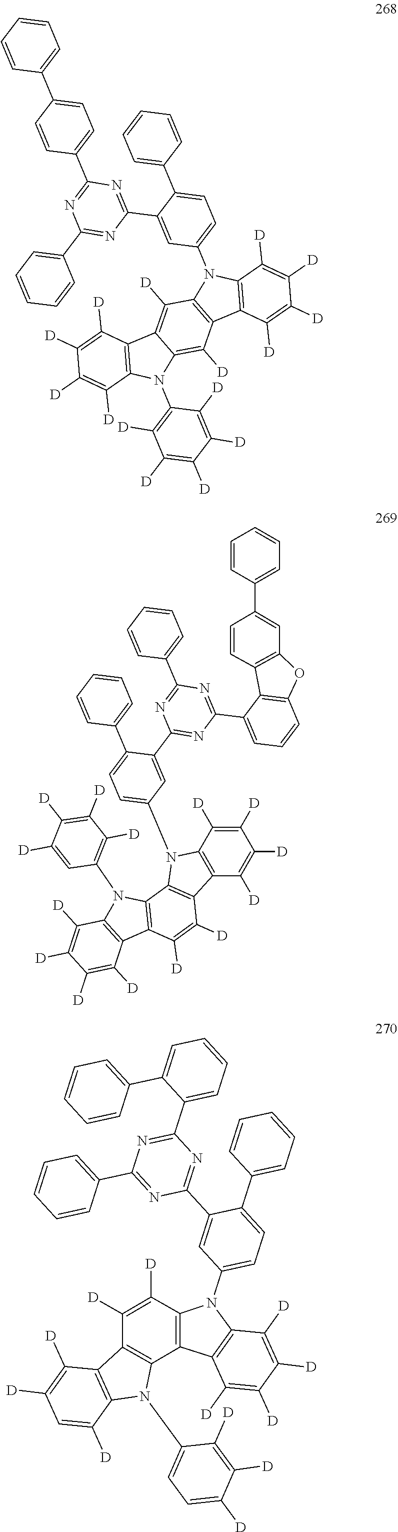

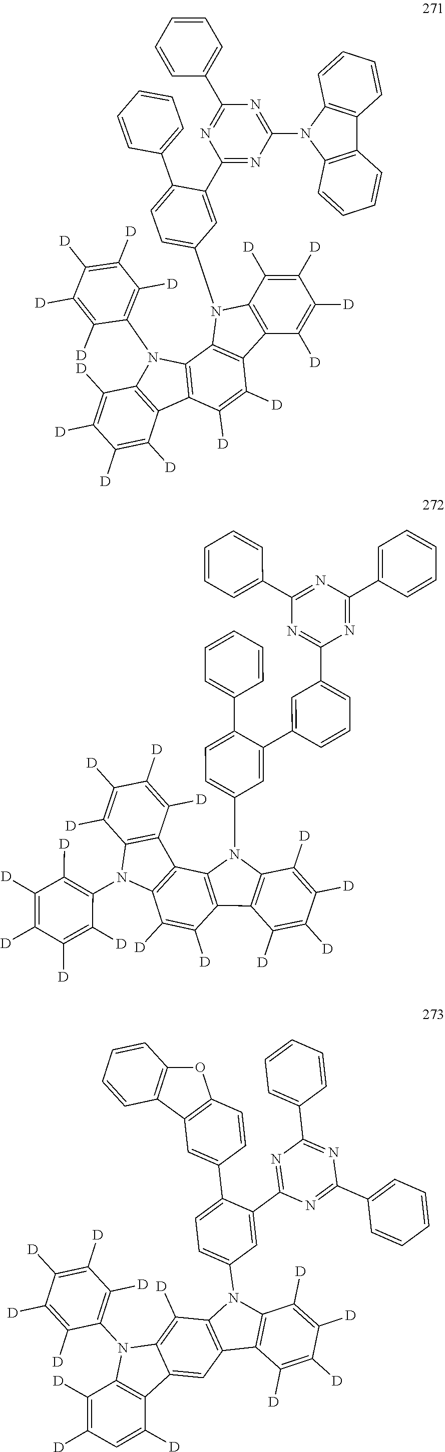

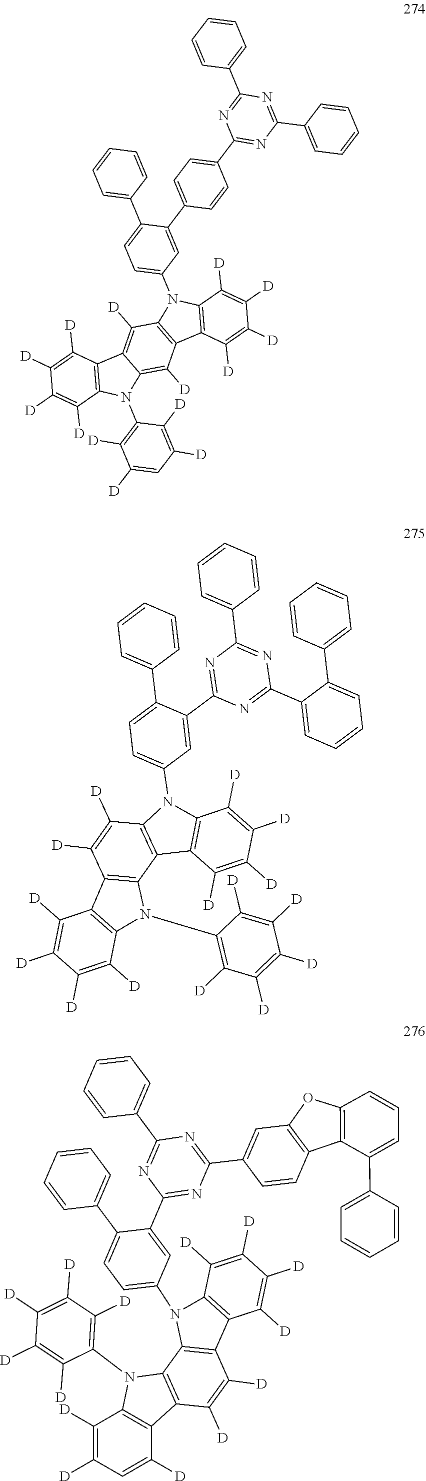

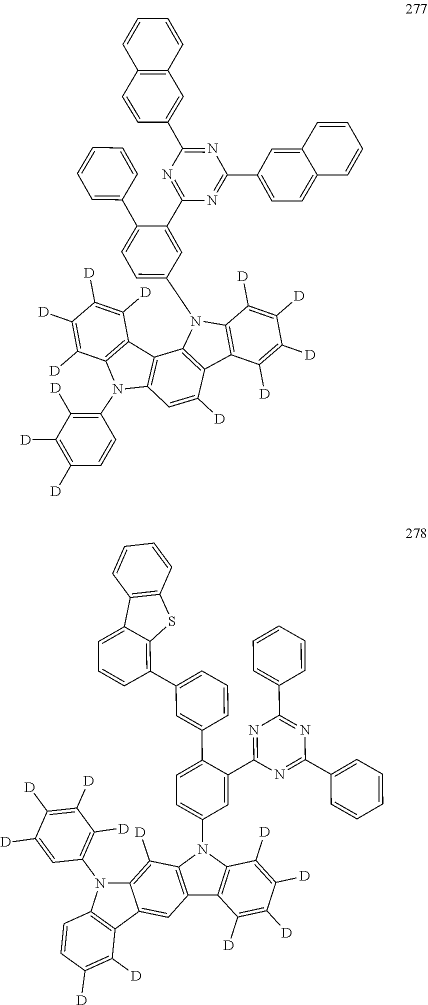 [SMILES: [2H]c1ccc(-n2c3cc([2H])c4c5c([2H])c([2H])c([2H])c([2H])c5n(-c5ccc(-c6ccccc6)c(-c6nc(-c7ccc8ccccc8c7)nc(-c7ccc8ccccc8c7)n6)c5)c4c3c3c([2H])c([2H])c([2H])c([2H])c32)c([2H])c1[2H] ; [2H]c1cc(-n2c3ccc([2H])c([2H])c3c3cc4c5c([2H])c([2H])c([2H])c([2H])c5n(-c5ccc(-c6cccc(-c7cccc8c7sc7ccccc78)c6)c(-c6nc(-c7ccccc7)nc(-c7ccccc7)n6)c5)c4c([2H])c32)c([2H])c([2H])c1[2H]]

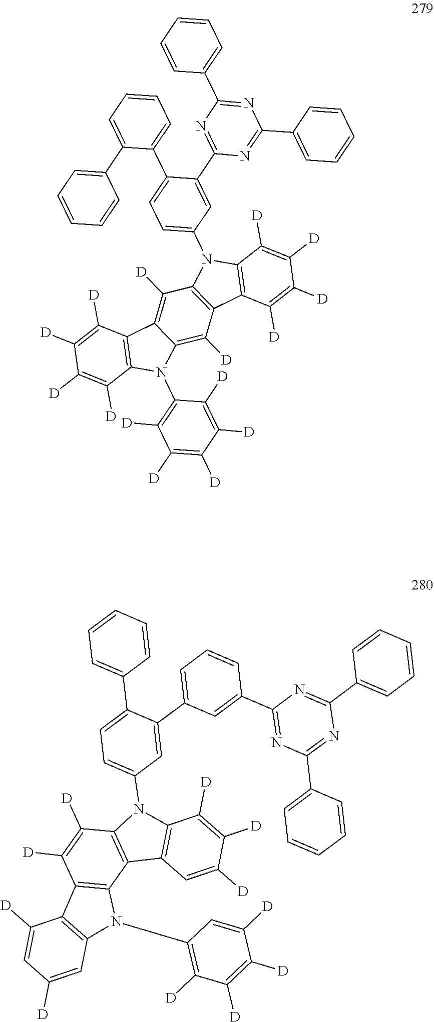 [SMILES: [2H]c1c([2H])c([2H])c(-n2c3c([2H])c([2H])c([2H])c([2H])c3c3c([2H])c4c(c([2H])c32)c2c([2H])c([2H])c([2H])c([2H])c2n4-c2ccc(-c3ccccc3-c3ccccc3)c(-c3nc(-c4ccccc4)nc(-c4ccccc4)n3)c2)c([2H])c1[2H] ; [2H]c1cc([2H])c2c3c([2H])c([2H])c4c(c5cc([2H])c([2H])c([2H])c5n4-c4ccc(-c5ccccc5)c(-c5cccc(-c6nc(-c7ccccc7)nc(-c7ccccc7)n6)c5)c4)c3n(-c3cc([2H])c([2H])c([2H])c3[2H])c2c1]

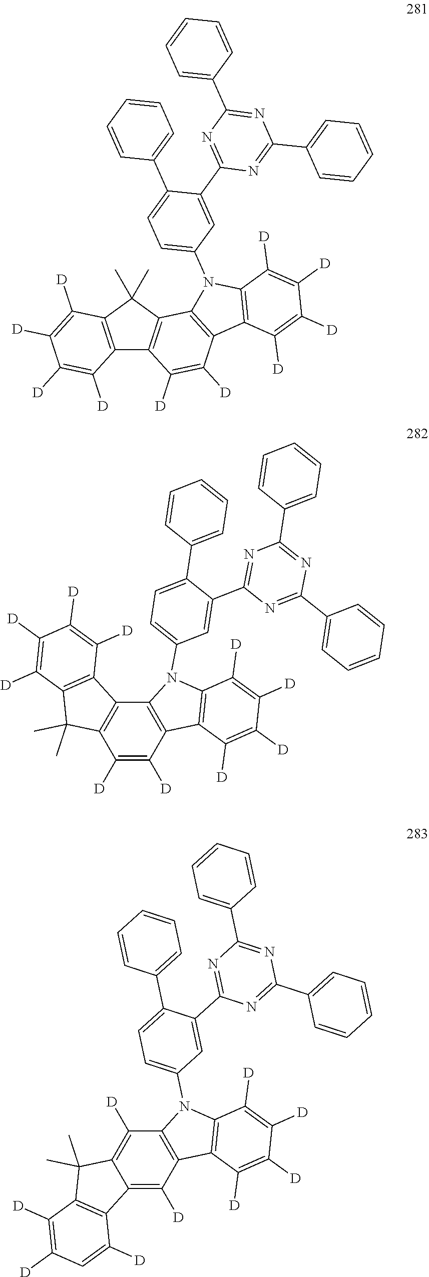 [SMILES: [2H]c1c([2H])c([2H])c2c(c1[2H])-c1c([2H])c([2H])c3c4c([2H])c([2H])c([2H])c([2H])c4n(-c4ccc(-c5ccccc5)c(-c5nc(-c6ccccc6)nc(-c6ccccc6)n5)c4)c3c1C2(C)C ; [2H]c1c([2H])c([2H])c2c(c1[2H])-c1c(c([2H])c([2H])c3c4c([2H])c([2H])c([2H])c([2H])c4n(-c4ccc(-c5ccccc5)c(-c5nc(-c6ccccc6)nc(-c6ccccc6)n5)c4)c13)C2(C)C ; [2H]c1cc([2H])c2c(c1[2H])C(C)(C)c1c-2c([2H])c2c3c([2H])c([2H])c([2H])c([2H])c3n(-c3ccc(-c4ccccc4)c(-c4nc(-c5ccccc5)nc(-c5ccccc5)n4)c3)c2c1[2H]]

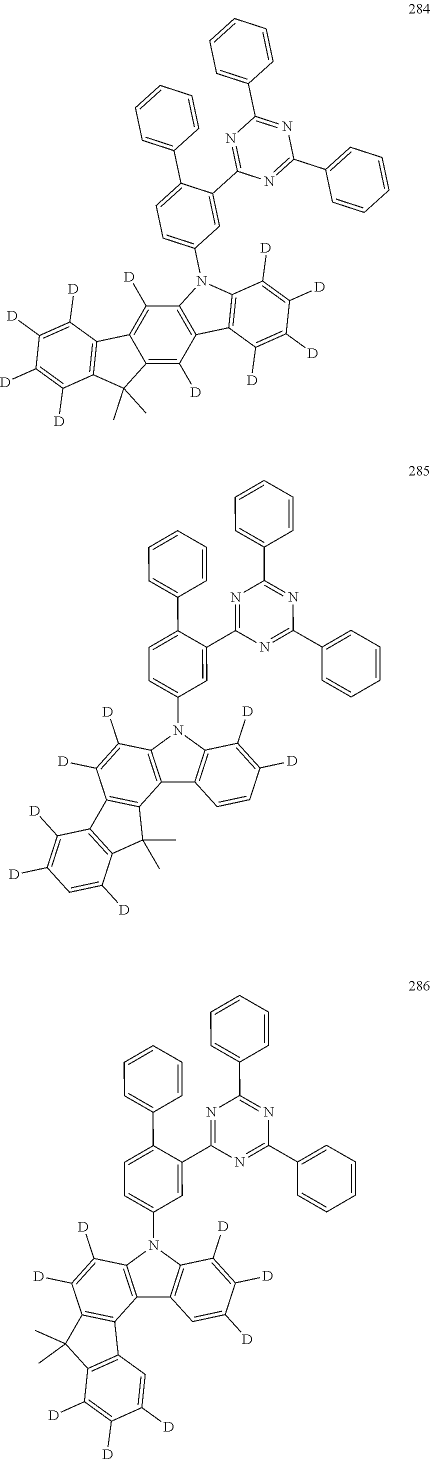 [SMILES: [2H]c1c([2H])c([2H])c2c(c1[2H])-c1c(c([2H])c3c4c([2H])c([2H])c([2H])c([2H])c4n(-c4ccc(-c5ccccc5)c(-c5nc(-c6ccccc6)nc(-c6ccccc6)n5)c4)c3c1[2H])C2(C)C ; [2H]c1cc([2H])c2c(c1[2H])-c1c([2H])c([2H])c3c(c1C2(C)C)c1ccc([2H])c([2H])c1n3-c1ccc(-c2ccccc2)c(-c2nc(-c3ccccc3)nc(-c3ccccc3)n2)c1 ; [2H]c1cc2c(c([2H])c1[2H])C(C)(C)c1c([2H])c([2H])c3c(c1-2)c1cc([2H])c([2H])c([2H])c1n3-c1ccc(-c2ccccc2)c(-c2nc(-c3ccccc3)nc(-c3ccccc3)n2)c1]

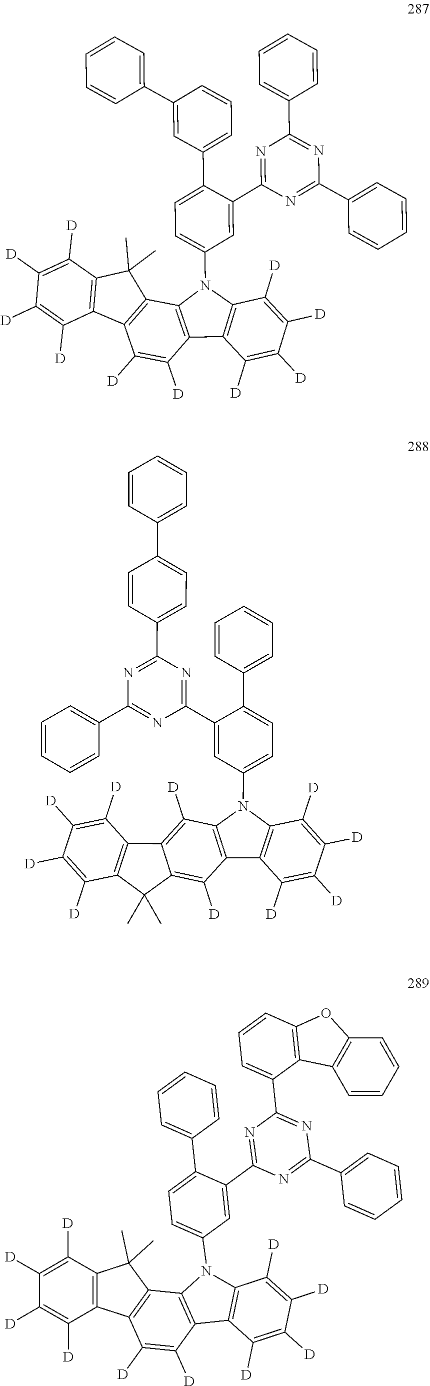 [SMILES: [2H]c1c([2H])c([2H])c2c(c1[2H])-c1c([2H])c([2H])c3c4c([2H])c([2H])c([2H])c([2H])c4n(-c4ccc(-c5cccc(-c6ccccc6)c5)c(-c5nc(-c6ccccc6)nc(-c6ccccc6)n5)c4)c3c1C2(C)C ; [2H]c1c([2H])c([2H])c2c(c1[2H])-c1c(c([2H])c3c4c([2H])c([2H])c([2H])c([2H])c4n(-c4ccc(-c5ccccc5)c(-c5nc(-c6ccccc6)nc(-c6ccc(-c7ccccc7)cc6)n5)c4)c3c1[2H])C2(C)C ; [2H]c1c([2H])c([2H])c2c(c1[2H])-c1c([2H])c([2H])c3c4c([2H])c([2H])c([2H])c([2H])c4n(-c4ccc(-c5ccccc5)c(-c5nc(-c6ccccc6)nc(-c6cccc7oc8ccccc8c67)n5)c4)c3c1C2(C)C]

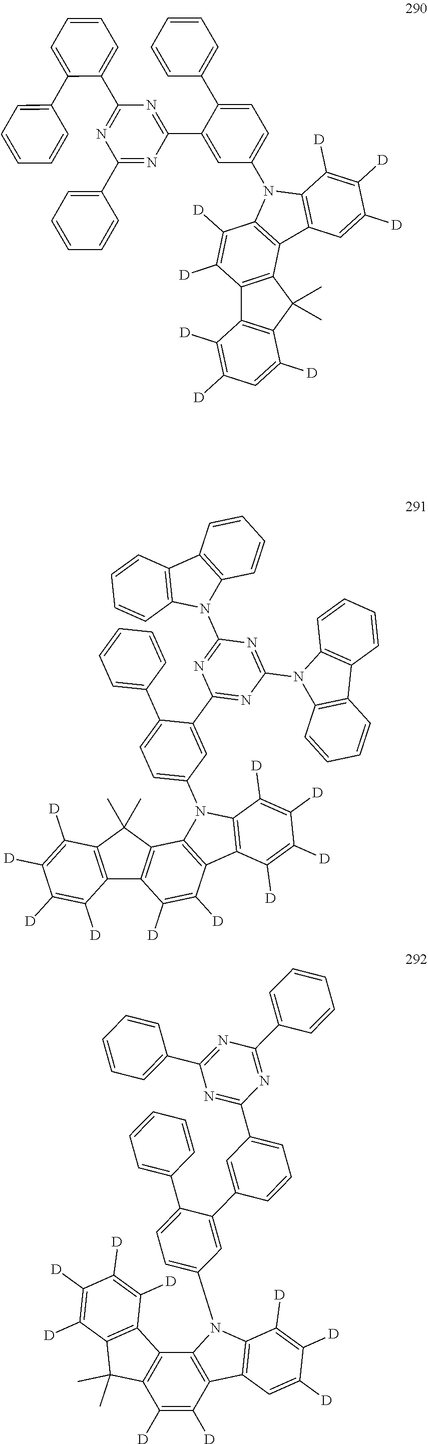 [SMILES: [2H]c1cc([2H])c2c(c1[2H])-c1c([2H])c([2H])c3c(c1C2(C)C)c1cc([2H])c([2H])c([2H])c1n3-c1ccc(-c2ccccc2)c(-c2nc(-c3ccccc3)nc(-c3ccccc3-c3ccccc3)n2)c1 ; [2H]c1c([2H])c([2H])c2c(c1[2H])-c1c([2H])c([2H])c3c4c([2H])c([2H])c([2H])c([2H])c4n(-c4ccc(-c5ccccc5)c(-c5nc(-n6c7ccccc7c7ccccc76)nc(-n6c7ccccc7c7ccccc76)n5)c4)c3c1C2(C)C ; [2H]c1cc2c3c([2H])c([2H])c4c(c3n(-c3ccc(-c5ccccc5)c(-c5cccc(-c6nc(-c7ccccc7)nc(-c7ccccc7)n6)c5)c3)c2c([2H])c1[2H])-c1c([2H])c([2H])c([2H])c([2H])c1C4(C)C]

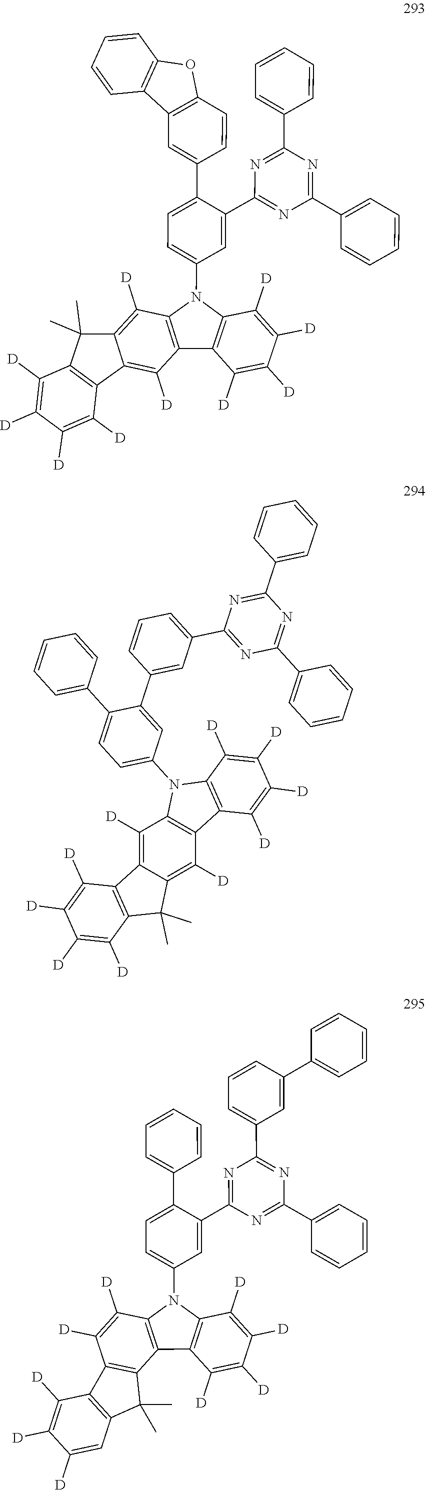 [SMILES: [2H]c1c([2H])c([2H])c2c(c1[2H])-c1c(c([2H])c3c(c1[2H])c1c([2H])c([2H])c([2H])c([2H])c1n3-c1ccc(-c3ccc4oc5ccccc5c4c3)c(-c3nc(-c4ccccc4)nc(-c4ccccc4)n3)c1)C2(C)C ; [2H]c1c([2H])c([2H])c2c(c1[2H])-c1c(c([2H])c3c4c([2H])c([2H])c([2H])c([2H])c4n(-c4ccc(-c5ccccc5)c(-c5cccc(-c6nc(-c7ccccc7)nc(-c7ccccc7)n6)c5)c4)c3c1[2H])C2(C)C ; [2H]c1cc2c(c([2H])c1[2H])-c1c([2H])c([2H])c3c(c1C2(C)C)c1c([2H])c([2H])c([2H])c([2H])c1n3-c1ccc(-c2ccccc2)c(-c2nc(-c3ccccc3)nc(-c3cccc(-c4ccccc4)c3)n2)c1]

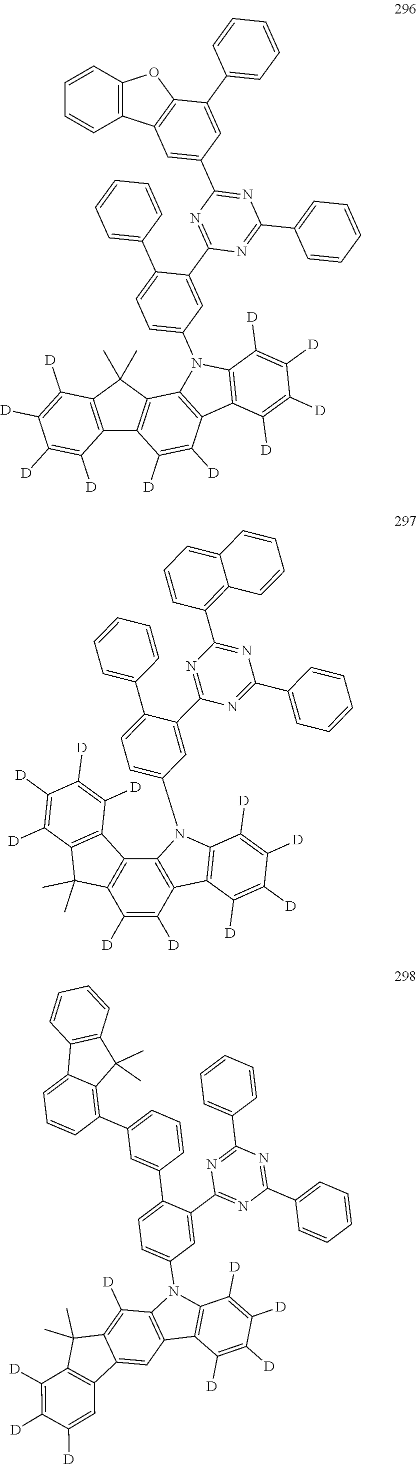

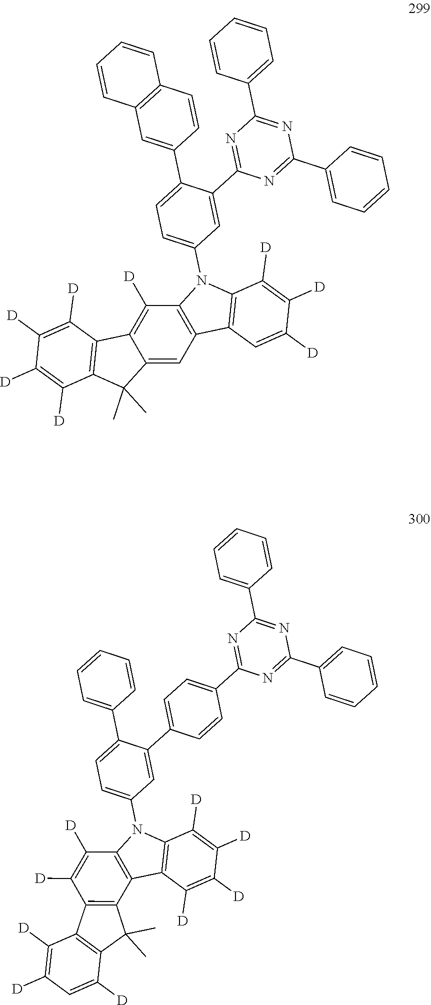 [SMILES: [2H]c1cc2c3cc4c(c([2H])c3n(-c3ccc(-c5ccc6ccccc6c5)c(-c5nc(-c6ccccc6)nc(-c6ccccc6)n5)c3)c2c([2H])c1[2H])-c1c([2H])c([2H])c([2H])c([2H])c1C4(C)C ; [2H]c1cc([2H])c2c(c1[2H])-c1c([2H])c([2H])c3c(c1C2(C)C)c1c([2H])c([2H])c([2H])c([2H])c1n3-c1ccc(-c2ccccc2)c(-c2ccc(-c3nc(-c4ccccc4)nc(-c4ccccc4)n3)cc2)c1]

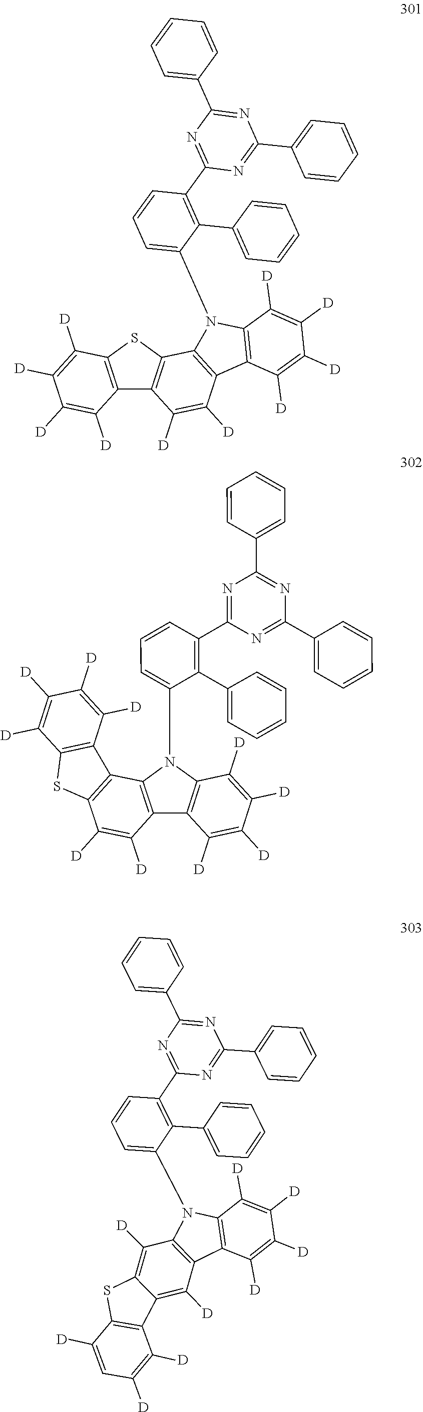 [SMILES: [2H]c1c([2H])c([2H])c2c(sc3c2c([2H])c([2H])c2c4c([2H])c([2H])c([2H])c([2H])c4n(-c4cccc(-c5nc(-c6ccccc6)nc(-c6ccccc6)n5)c4-c4ccccc4)c32)c1[2H] ; [2H]c1c([2H])c([2H])c2c(sc3c([2H])c([2H])c4c5c([2H])c([2H])c([2H])c([2H])c5n(-c5cccc(-c6nc(-c7ccccc7)nc(-c7ccccc7)n6)c5-c5ccccc5)c4c32)c1[2H] ; [2H]c1cc([2H])c2sc3c([2H])c4c(c([2H])c3c2c1[2H])c1c([2H])c([2H])c([2H])c([2H])c1n4-c1cccc(-c2nc(-c3ccccc3)nc(-c3ccccc3)n2)c1-c1ccccc1]

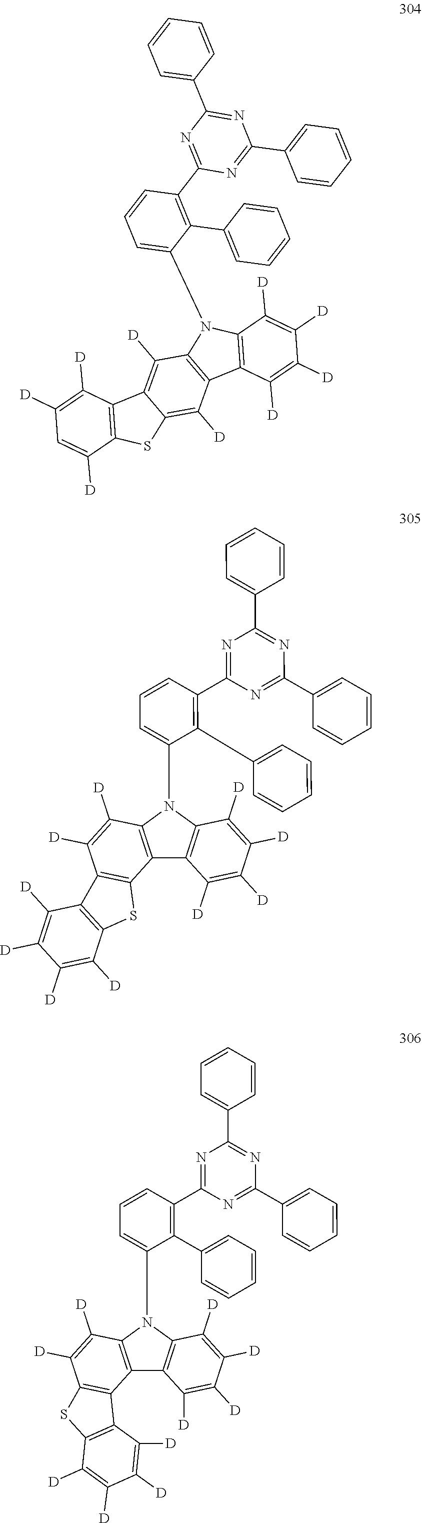 [SMILES: [2H]c1cc([2H])c2sc3c([2H])c4c5c([2H])c([2H])c([2H])c([2H])c5n(-c5cccc(-c6nc(-c7ccccc7)nc(-c7ccccc7)n6)c5-c5ccccc5)c4c([2H])c3c2c1[2H] ; [2H]c1c([2H])c([2H])c2c(sc3c2c([2H])c([2H])c2c3c3c([2H])c([2H])c([2H])c([2H])c3n2-c2cccc(-c3nc(-c4ccccc4)nc(-c4ccccc4)n3)c2-c2ccccc2)c1[2H] ; [2H]c1c([2H])c([2H])c2c(sc3c([2H])c([2H])c4c(c5c([2H])c([2H])c([2H])c([2H])c5n4-c4cccc(-c5nc(-c6ccccc6)nc(-c6ccccc6)n5)c4-c4ccccc4)c32)c1[2H]]

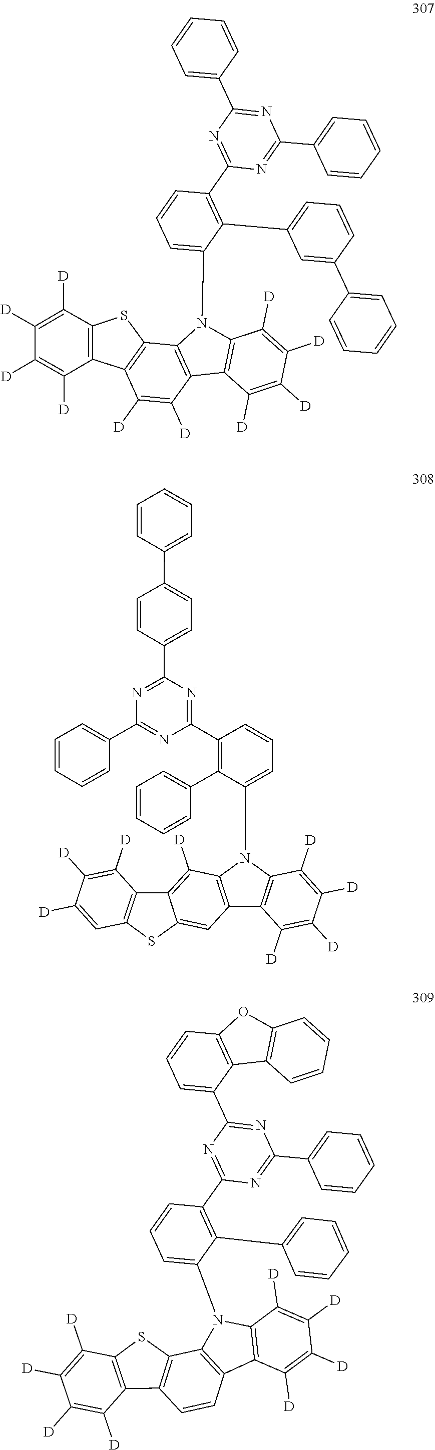 [SMILES: [2H]c1c([2H])c([2H])c2c(sc3c2c([2H])c([2H])c2c4c([2H])c([2H])c([2H])c([2H])c4n(-c4cccc(-c5nc(-c6ccccc6)nc(-c6ccccc6)n5)c4-c4cccc(-c5ccccc5)c4)c32)c1[2H] ; [2H]c1cc2sc3cc4c5c([2H])c([2H])c([2H])c([2H])c5n(-c5cccc(-c6nc(-c7ccccc7)nc(-c7ccc(-c8ccccc8)cc7)n6)c5-c5ccccc5)c4c([2H])c3c2c([2H])c1[2H] ; [2H]c1c([2H])c([2H])c2c(sc3c2ccc2c4c([2H])c([2H])c([2H])c([2H])c4n(-c4cccc(-c5nc(-c6ccccc6)nc(-c6cccc7oc8ccccc8c67)n5)c4-c4ccccc4)c23)c1[2H]]

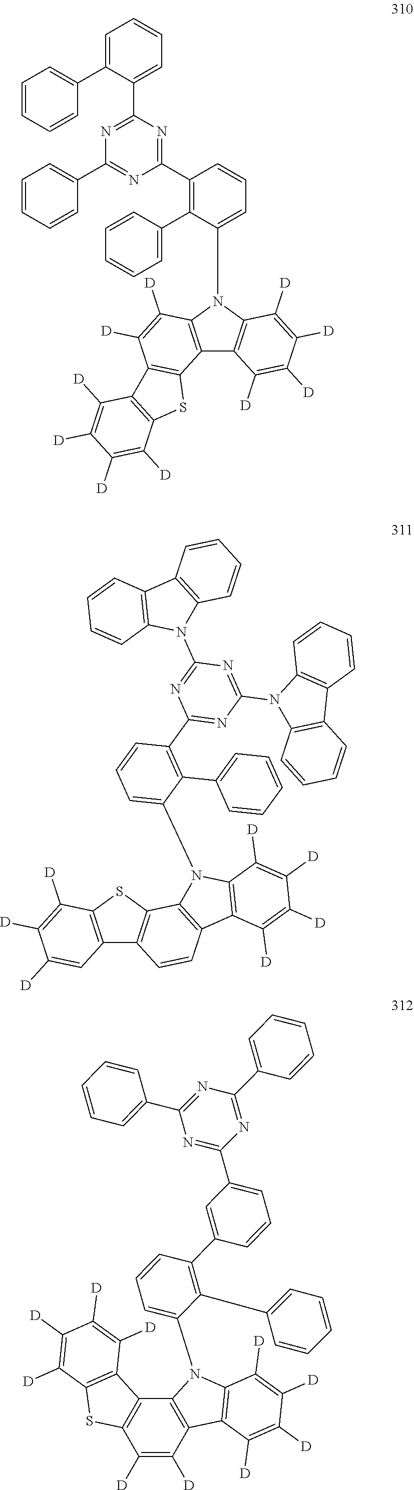 [SMILES: [2H]c1c([2H])c([2H])c2c(sc3c2c([2H])c([2H])c2c3c3c([2H])c([2H])c([2H])c([2H])c3n2-c2cccc(-c3nc(-c4ccccc4)nc(-c4ccccc4-c4ccccc4)n3)c2-c2ccccc2)c1[2H] ; [2H]c1cc2c(sc3c2ccc2c4c([2H])c([2H])c([2H])c([2H])c4n(-c4cccc(-c5nc(-n6c7ccccc7c7ccccc76)nc(-n6c7ccccc7c7ccccc76)n5)c4-c4ccccc4)c23)c([2H])c1[2H] ; [2H]c1c([2H])c([2H])c2c(sc3c([2H])c([2H])c4c5c([2H])c([2H])c([2H])c([2H])c5n(-c5cccc(-c6cccc(-c7nc(-c8ccccc8)nc(-c8ccccc8)n7)c6)c5-c5ccccc5)c4c32)c1[2H]]

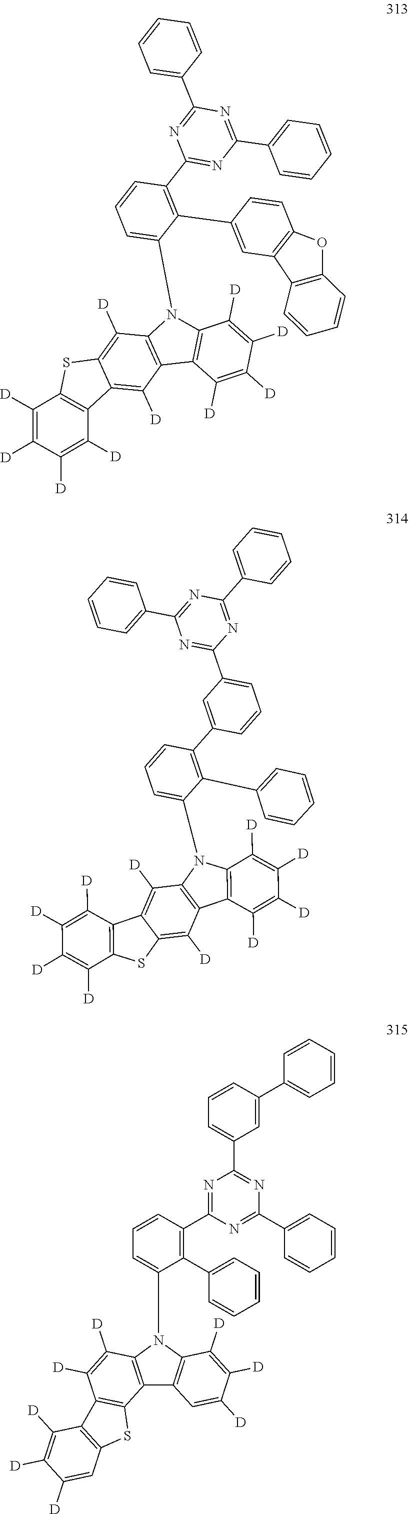 [SMILES: [2H]c1c([2H])c([2H])c2c(sc3c([2H])c4c(c([2H])c32)c2c([2H])c([2H])c([2H])c([2H])c2n4-c2cccc(-c3nc(-c4ccccc4)nc(-c4ccccc4)n3)c2-c2ccc3oc4ccccc4c3c2)c1[2H] ; [2H]c1c([2H])c([2H])c2c(sc3c([2H])c4c5c([2H])c([2H])c([2H])c([2H])c5n(-c5cccc(-c6cccc(-c7nc(-c8ccccc8)nc(-c8ccccc8)n7)c6)c5-c5ccccc5)c4c([2H])c32)c1[2H] ; [2H]c1cc2sc3c(c([2H])c([2H])c4c3c3cc([2H])c([2H])c([2H])c3n4-c3cccc(-c4nc(-c5ccccc5)nc(-c5cccc(-c6ccccc6)c5)n4)c3-c3ccccc3)c2c([2H])c1[2H]]

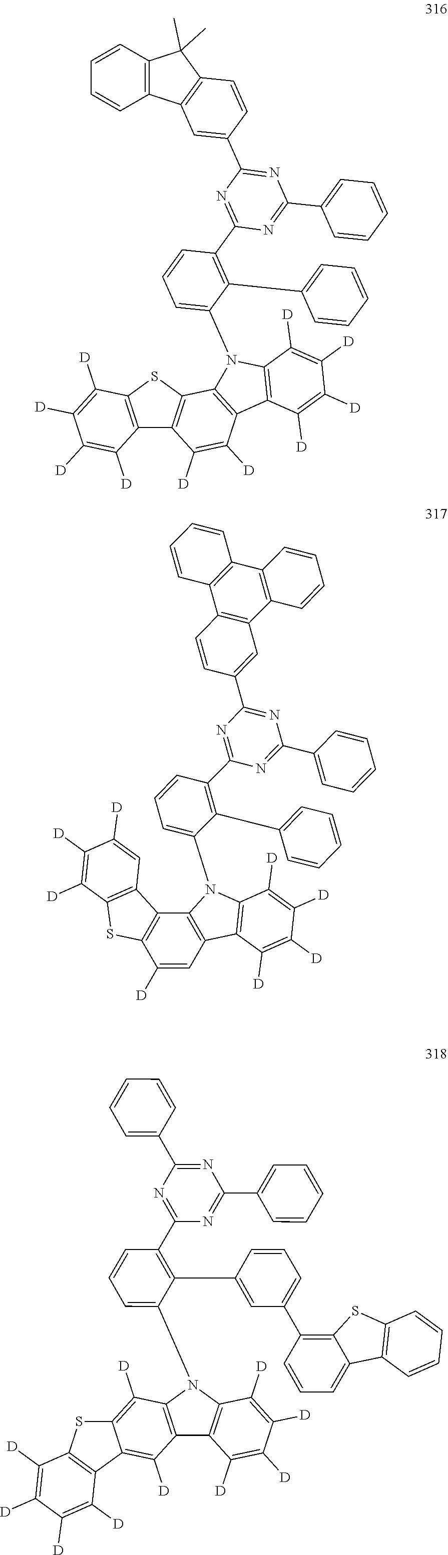

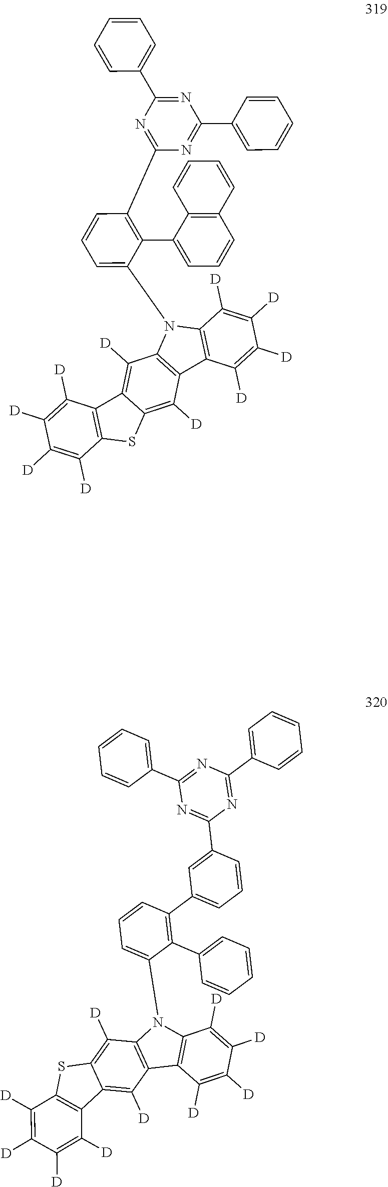 [SMILES: [2H]c1c([2H])c([2H])c2c(sc3c([2H])c4c5c([2H])c([2H])c([2H])c([2H])c5n(-c5cccc(-c6nc(-c7ccccc7)nc(-c7ccccc7)n6)c5-c5cccc6ccccc56)c4c([2H])c32)c1[2H] ; [2H]c1c([2H])c([2H])c2c(sc3c([2H])c4c(c([2H])c32)c2c([2H])c([2H])c([2H])c([2H])c2n4-c2cccc(-c3cccc(-c4nc(-c5ccccc5)nc(-c5ccccc5)n4)c3)c2-c2ccccc2)c1[2H]]

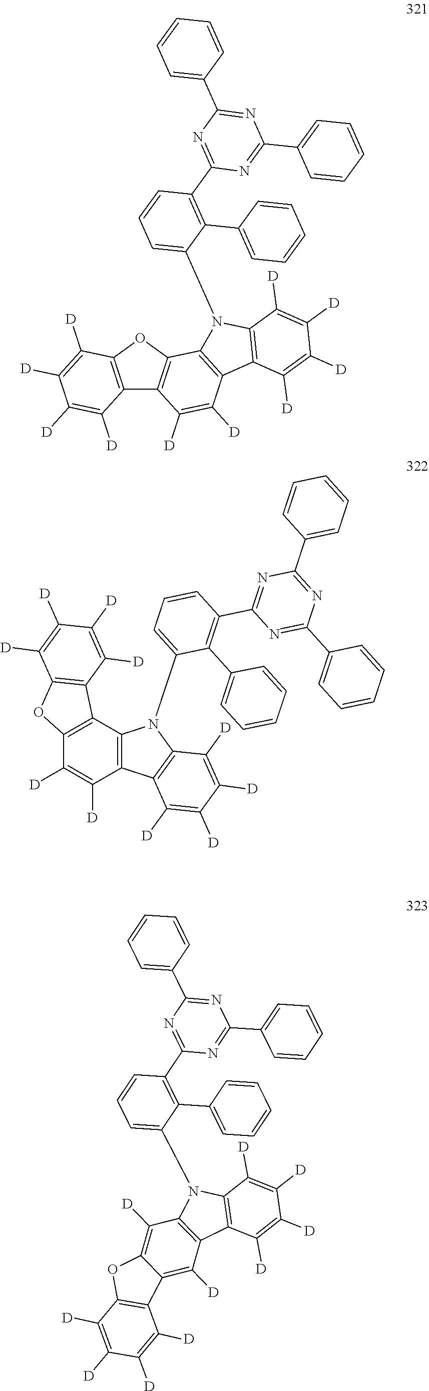 [SMILES: [2H]c1c([2H])c([2H])c2c(oc3c2c([2H])c([2H])c2c4c([2H])c([2H])c([2H])c([2H])c4n(-c4cccc(-c5nc(-c6ccccc6)nc(-c6ccccc6)n5)c4-c4ccccc4)c32)c1[2H] ; [2H]c1c([2H])c([2H])c2c(oc3c([2H])c([2H])c4c5c([2H])c([2H])c([2H])c([2H])c5n(-c5cccc(-c6nc(-c7ccccc7)nc(-c7ccccc7)n6)c5-c5ccccc5)c4c32)c1[2H] ; [2H]c1c([2H])c([2H])c2c(oc3c([2H])c4c(c([2H])c32)c2c([2H])c([2H])c([2H])c([2H])c2n4-c2cccc(-c3nc(-c4ccccc4)nc(-c4ccccc4)n3)c2-c2ccccc2)c1[2H]]

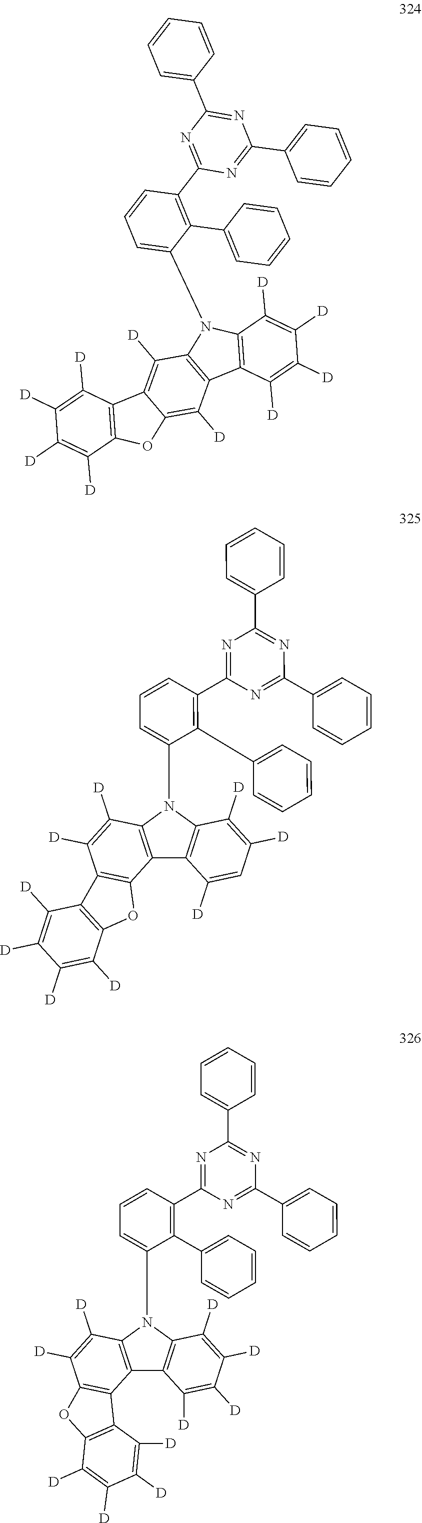 [SMILES: [2H]c1c([2H])c([2H])c2c(oc3c([2H])c4c5c([2H])c([2H])c([2H])c([2H])c5n(-c5cccc(-c6nc(-c7ccccc7)nc(-c7ccccc7)n6)c5-c5ccccc5)c4c([2H])c32)c1[2H] ; [2H]c1cc([2H])c2c3c4oc5c([2H])c([2H])c([2H])c([2H])c5c4c([2H])c([2H])c3n(-c3cccc(-c4nc(-c5ccccc5)nc(-c5ccccc5)n4)c3-c3ccccc3)c2c1[2H] ; [2H]c1c([2H])c([2H])c2c(oc3c([2H])c([2H])c4c(c5c([2H])c([2H])c([2H])c([2H])c5n4-c4cccc(-c5nc(-c6ccccc6)nc(-c6ccccc6)n5)c4-c4ccccc4)c32)c1[2H]]

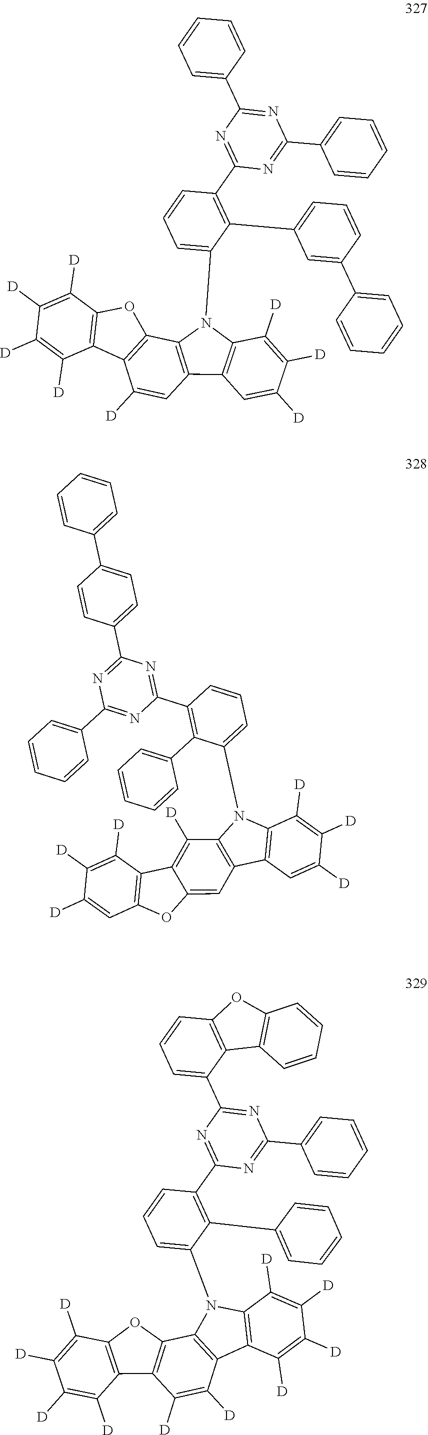 [SMILES: [2H]c1cc2c3cc([2H])c4c(oc5c([2H])c([2H])c([2H])c([2H])c54)c3n(-c3cccc(-c4nc(-c5ccccc5)nc(-c5ccccc5)n4)c3-c3cccc(-c4ccccc4)c3)c2c([2H])c1[2H] ; [2H]c1cc2oc3cc4c5cc([2H])c([2H])c([2H])c5n(-c5cccc(-c6nc(-c7ccccc7)nc(-c7ccc(-c8ccccc8)cc7)n6)c5-c5ccccc5)c4c([2H])c3c2c([2H])c1[2H] ; [2H]c1c([2H])c([2H])c2c(oc3c2c([2H])c([2H])c2c4c([2H])c([2H])c([2H])c([2H])c4n(-c4cccc(-c5nc(-c6ccccc6)nc(-c6cccc7oc8ccccc8c67)n5)c4-c4ccccc4)c32)c1[2H]]

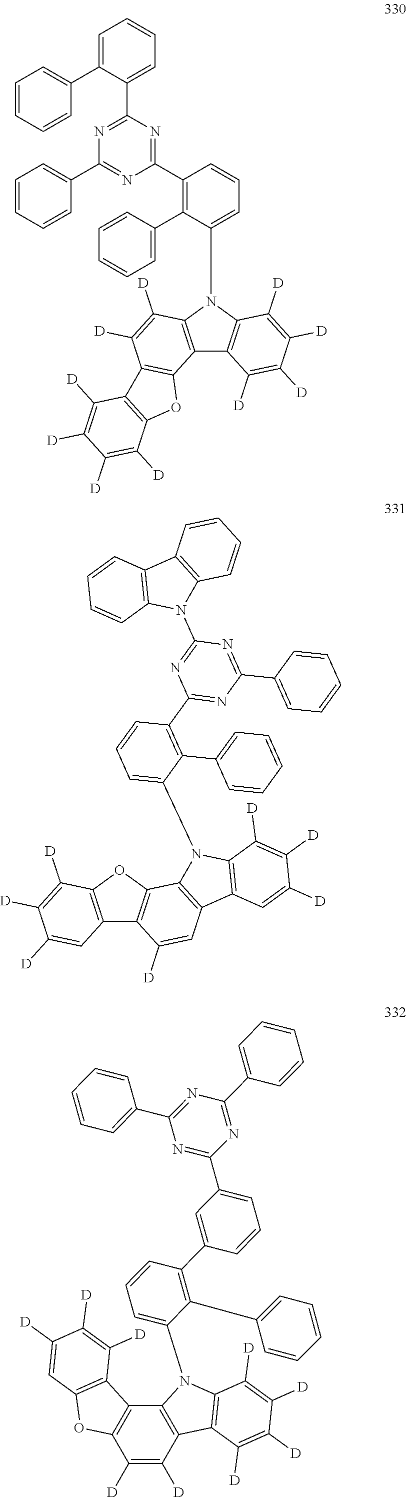 [SMILES: [2H]c1c([2H])c([2H])c2c(oc3c2c([2H])c([2H])c2c3c3c([2H])c([2H])c([2H])c([2H])c3n2-c2cccc(-c3nc(-c4ccccc4)nc(-c4ccccc4-c4ccccc4)n3)c2-c2ccccc2)c1[2H] ; [2H]c1cc2c(oc3c2c([2H])cc2c4cc([2H])c([2H])c([2H])c4n(-c4cccc(-c5nc(-c6ccccc6)nc(-n6c7ccccc7c7ccccc76)n5)c4-c4ccccc4)c23)c([2H])c1[2H] ; [2H]c1cc2oc3c([2H])c([2H])c4c5c([2H])c([2H])c([2H])c([2H])c5n(-c5cccc(-c6cccc(-c7nc(-c8ccccc8)nc(-c8ccccc8)n7)c6)c5-c5ccccc5)c4c3c2c([2H])c1[2H]]

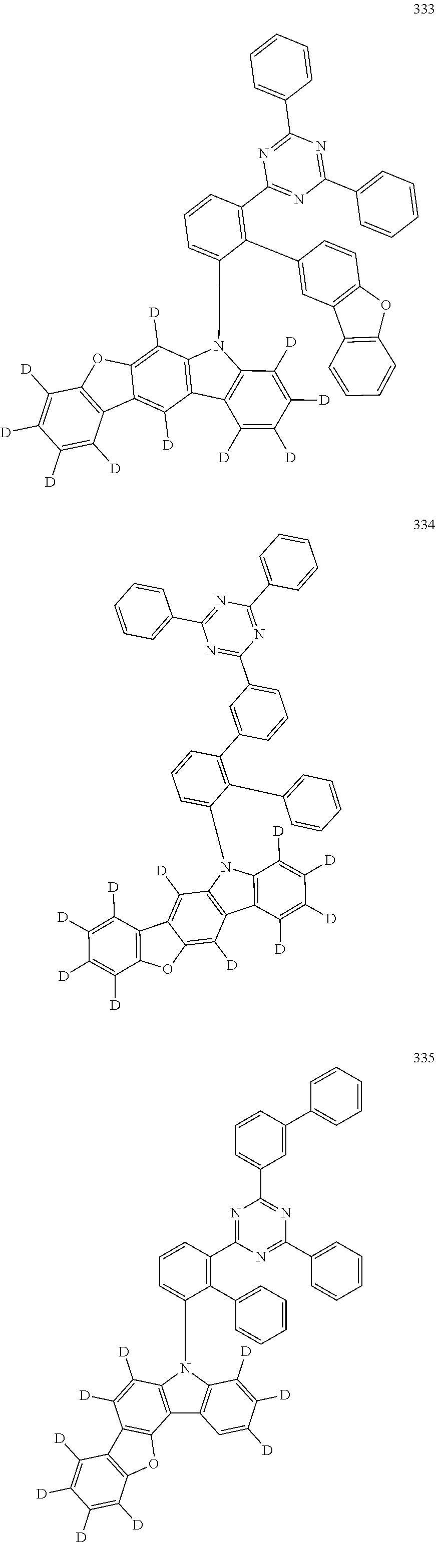 [SMILES: [2H]c1c([2H])c([2H])c2c(oc3c([2H])c4c(c([2H])c32)c2c([2H])c([2H])c([2H])c([2H])c2n4-c2cccc(-c3nc(-c4ccccc4)nc(-c4ccccc4)n3)c2-c2ccc3oc4ccccc4c3c2)c1[2H] ; [2H]c1c([2H])c([2H])c2c(oc3c([2H])c4c5c([2H])c([2H])c([2H])c([2H])c5n(-c5cccc(-c6cccc(-c7nc(-c8ccccc8)nc(-c8ccccc8)n7)c6)c5-c5ccccc5)c4c([2H])c32)c1[2H] ; [2H]c1cc2c3c4oc5c([2H])c([2H])c([2H])c([2H])c5c4c([2H])c([2H])c3n(-c3cccc(-c4nc(-c5ccccc5)nc(-c5cccc(-c6ccccc6)c5)n4)c3-c3ccccc3)c2c([2H])c1[2H]]

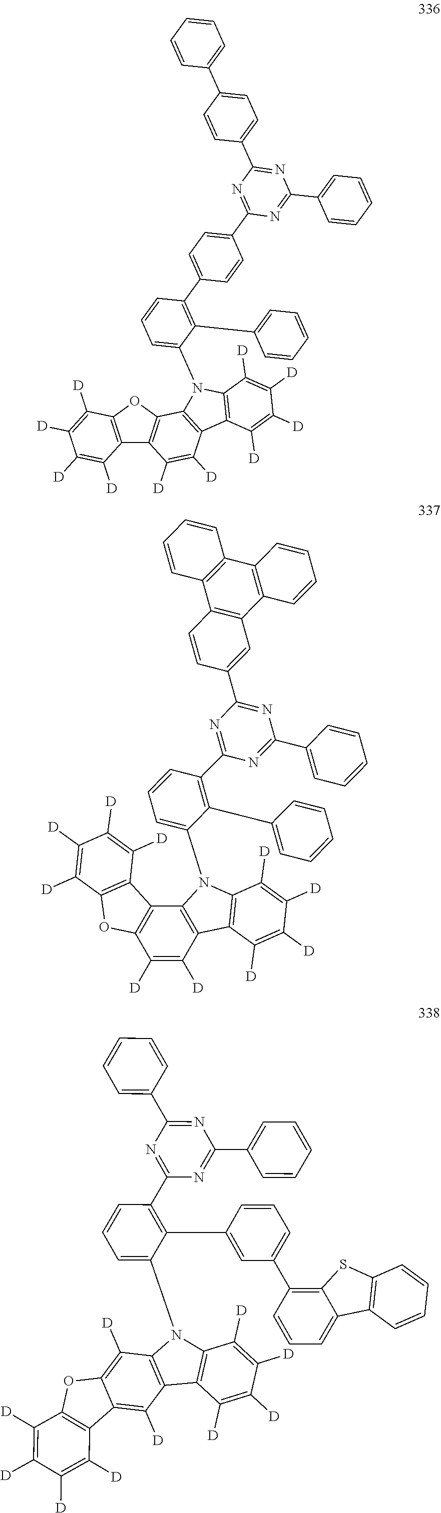

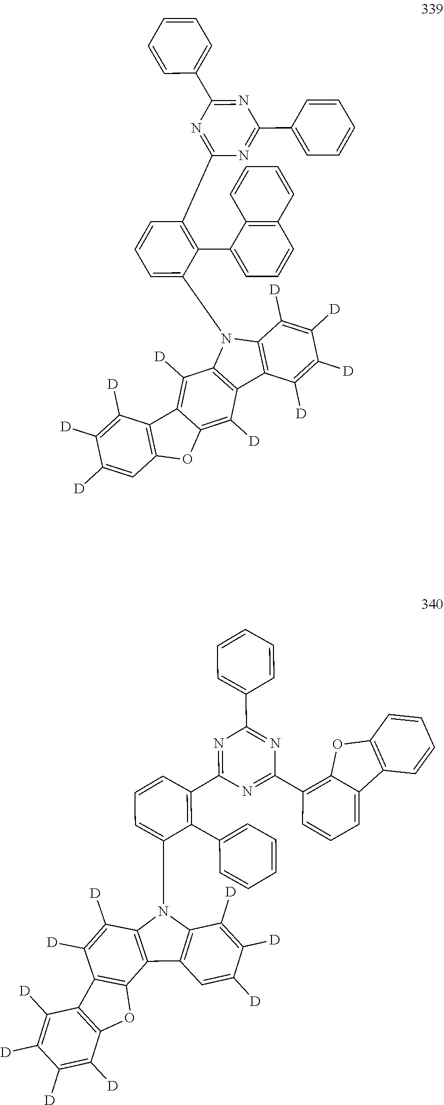 [SMILES: [2H]c1cc2oc3c([2H])c4c5c([2H])c([2H])c([2H])c([2H])c5n(-c5cccc(-c6nc(-c7ccccc7)nc(-c7ccccc7)n6)c5-c5cccc6ccccc56)c4c([2H])c3c2c([2H])c1[2H] ; [2H]c1cc2c3c4oc5c([2H])c([2H])c([2H])c([2H])c5c4c([2H])c([2H])c3n(-c3cccc(-c4nc(-c5ccccc5)nc(-c5cccc6c5oc5ccccc56)n4)c3-c3ccccc3)c2c([2H])c1[2H]]

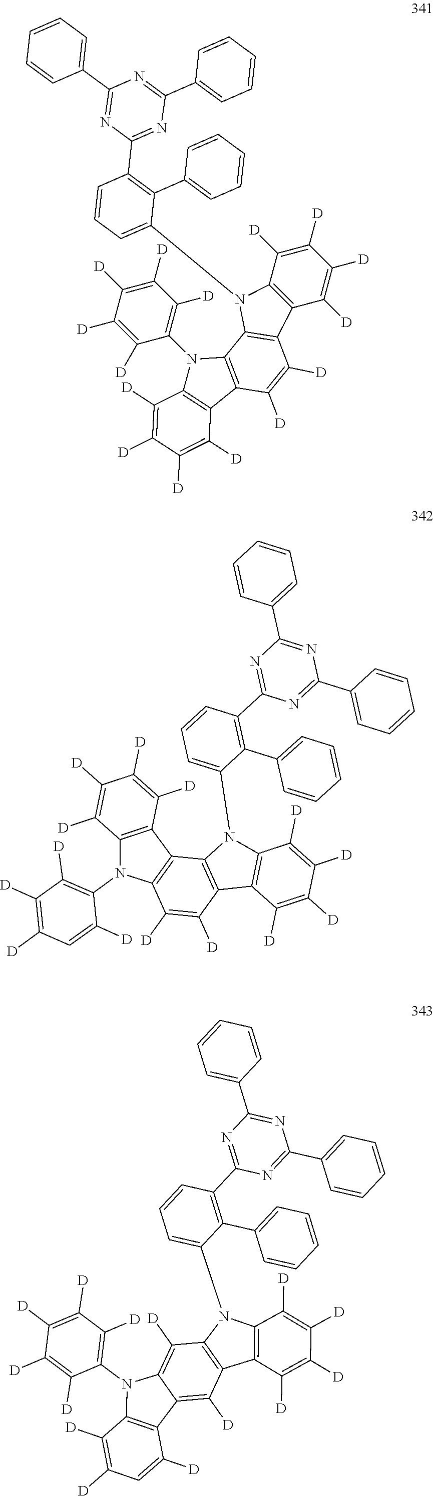

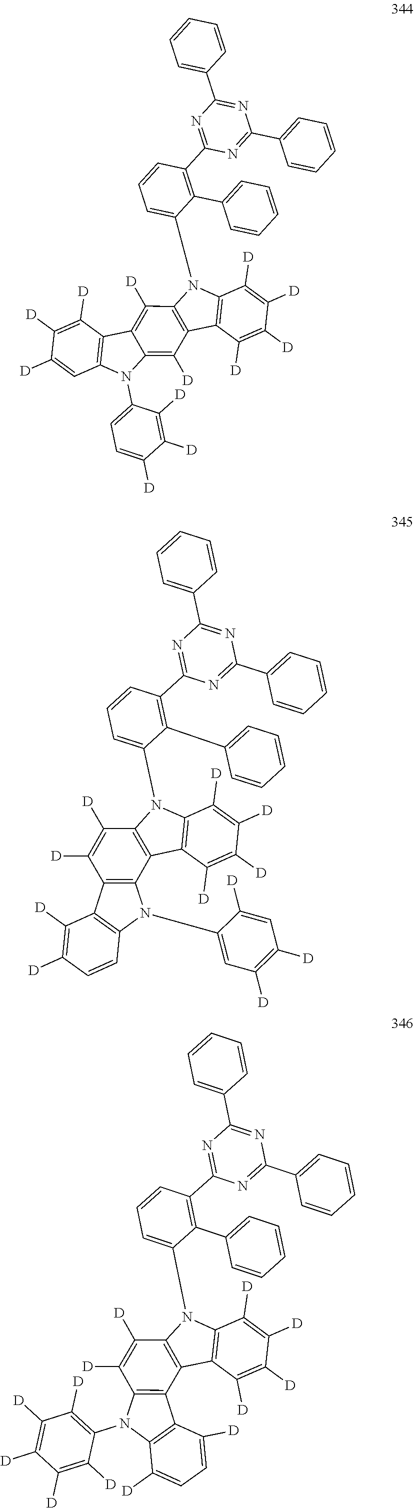 [SMILES: [2H]c1ccc(-n2c3cc([2H])c([2H])c([2H])c3c3c([2H])c4c(c([2H])c32)c2c([2H])c([2H])c([2H])c([2H])c2n4-c2cccc(-c3nc(-c4ccccc4)nc(-c4ccccc4)n3)c2-c2ccccc2)c([2H])c1[2H] ; [2H]c1cc([2H])c(-n2c3ccc([2H])c([2H])c3c3c([2H])c([2H])c4c(c5c([2H])c([2H])c([2H])c([2H])c5n4-c4cccc(-c5nc(-c6ccccc6)nc(-c6ccccc6)n5)c4-c4ccccc4)c32)cc1[2H] ; [2H]c1c([2H])c([2H])c(-n2c3c([2H])ccc([2H])c3c3c4c5c([2H])c([2H])c([2H])c([2H])c5n(-c5cccc(-c6nc(-c7ccccc7)nc(-c7ccccc7)n6)c5-c5ccccc5)c4c([2H])c([2H])c32)c([2H])c1[2H]]

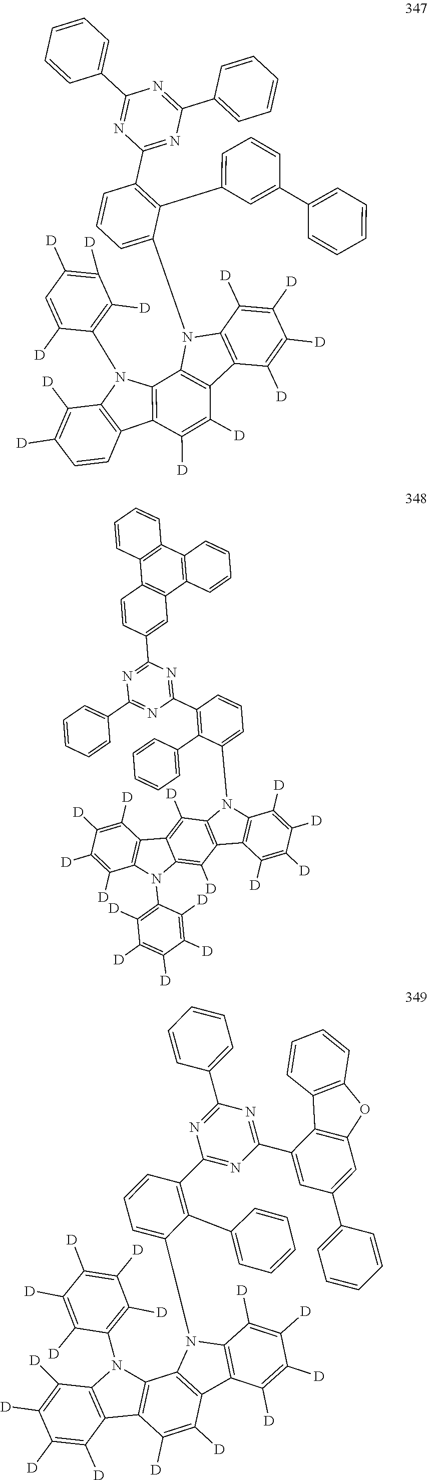

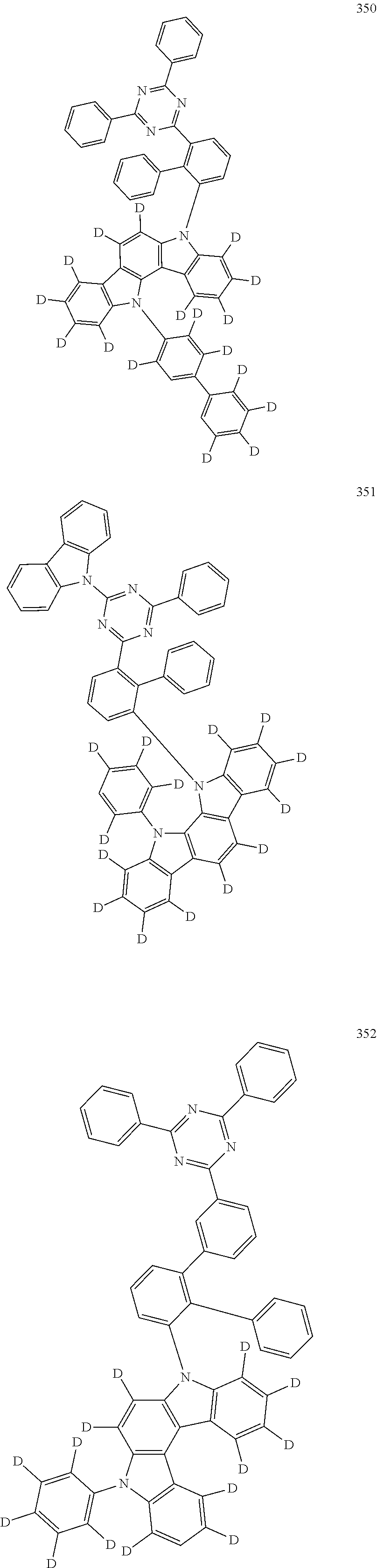

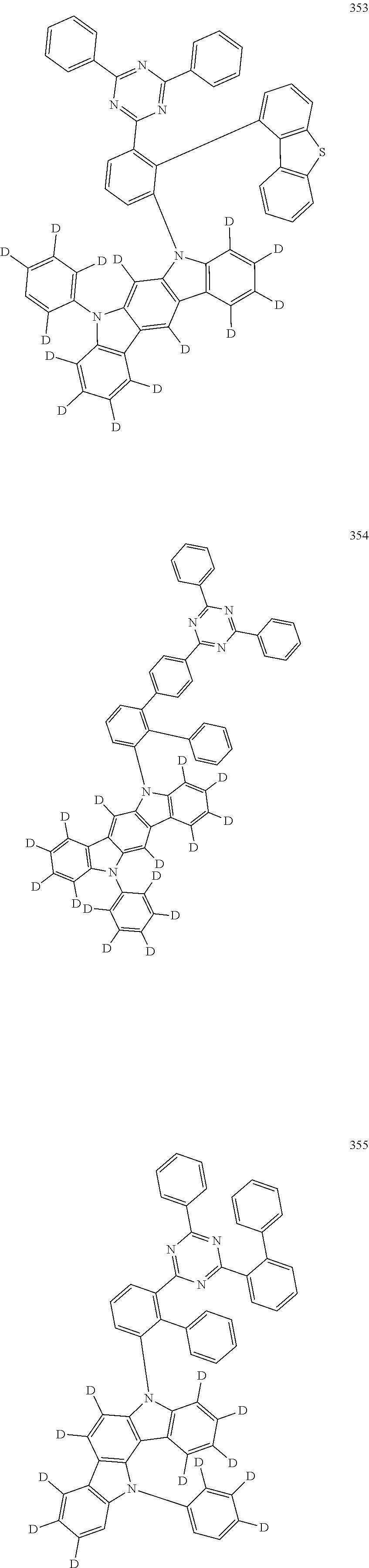

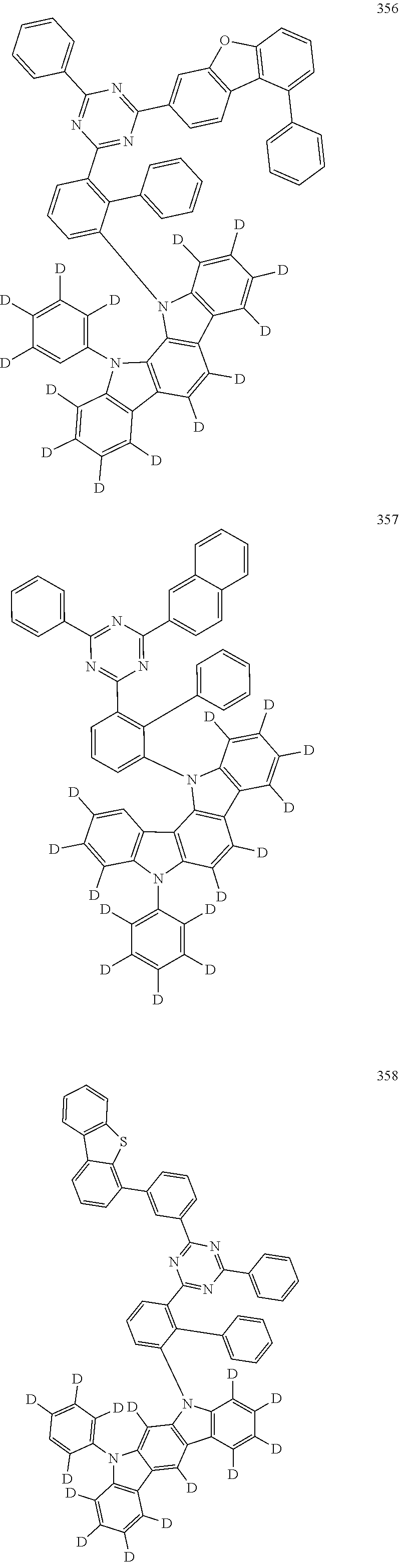

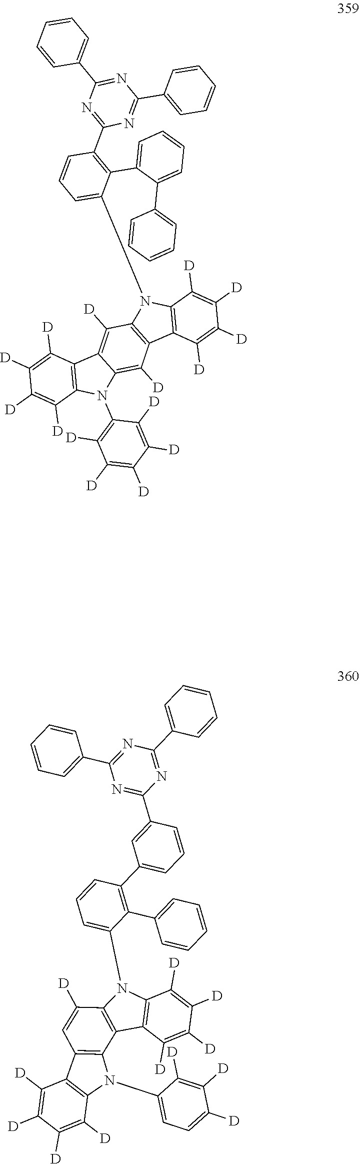 [SMILES: [2H]c1c([2H])c([2H])c(-n2c3c([2H])c([2H])c([2H])c([2H])c3c3c([2H])c4c(c([2H])c32)c2c([2H])c([2H])c([2H])c([2H])c2n4-c2cccc(-c3nc(-c4ccccc4)nc(-c4ccccc4)n3)c2-c2ccccc2-c2ccccc2)c([2H])c1[2H] ; [2H]c1ccc(-n2c3c([2H])c([2H])c([2H])c([2H])c3c3cc([2H])c4c(c5c([2H])c([2H])c([2H])c([2H])c5n4-c4cccc(-c5cccc(-c6nc(-c7ccccc7)nc(-c7ccccc7)n6)c5)c4-c4ccccc4)c32)c([2H])c1[2H]]

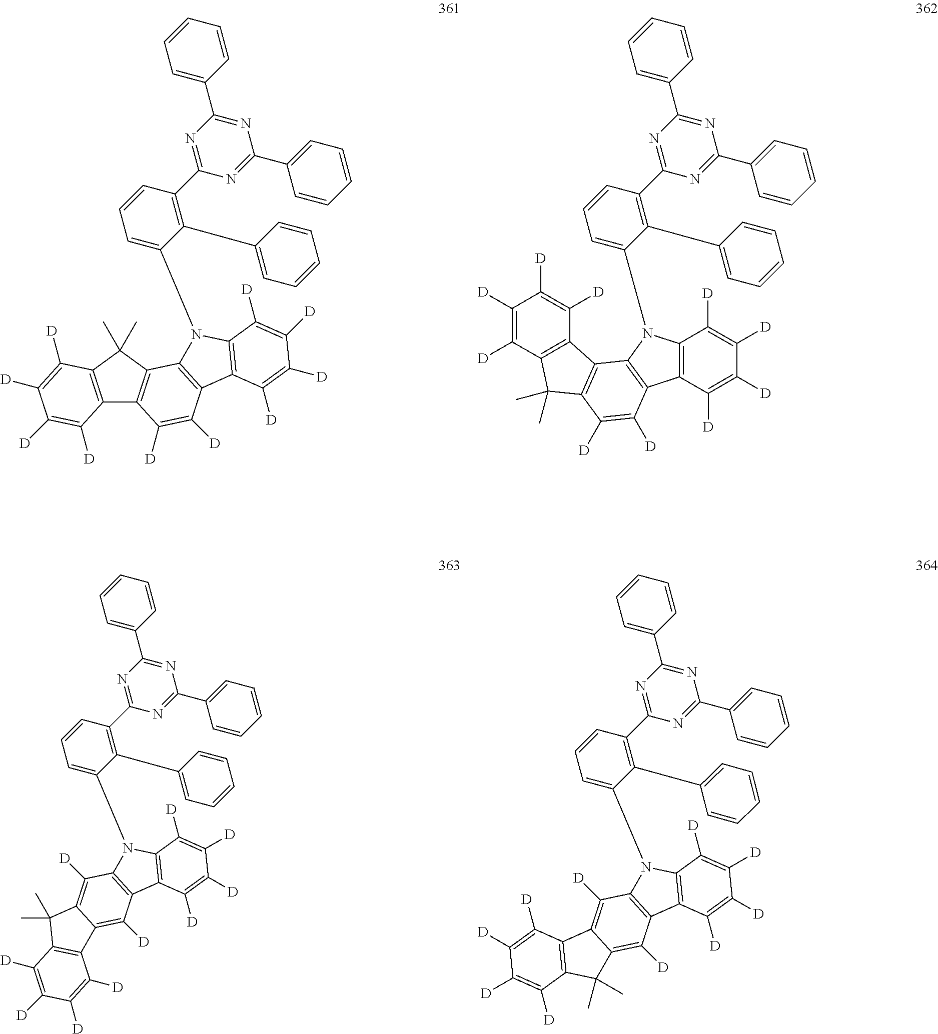

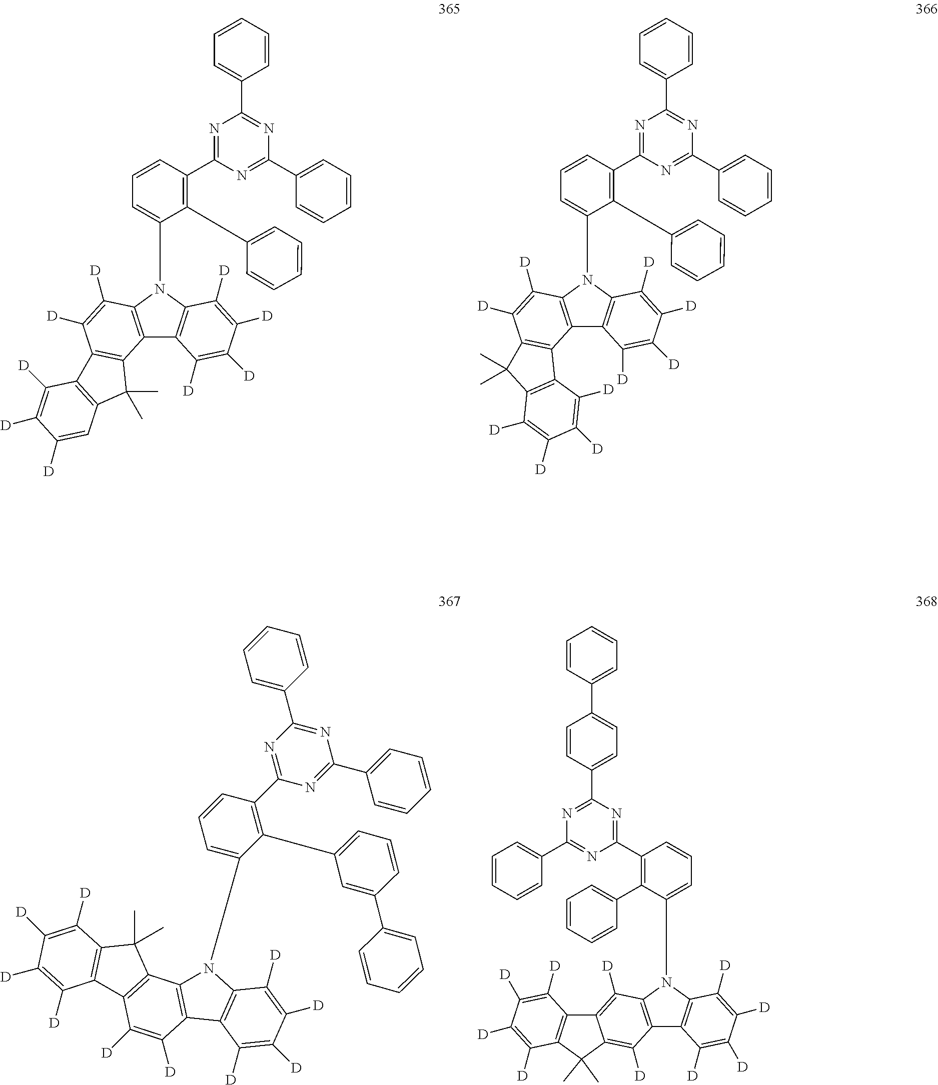

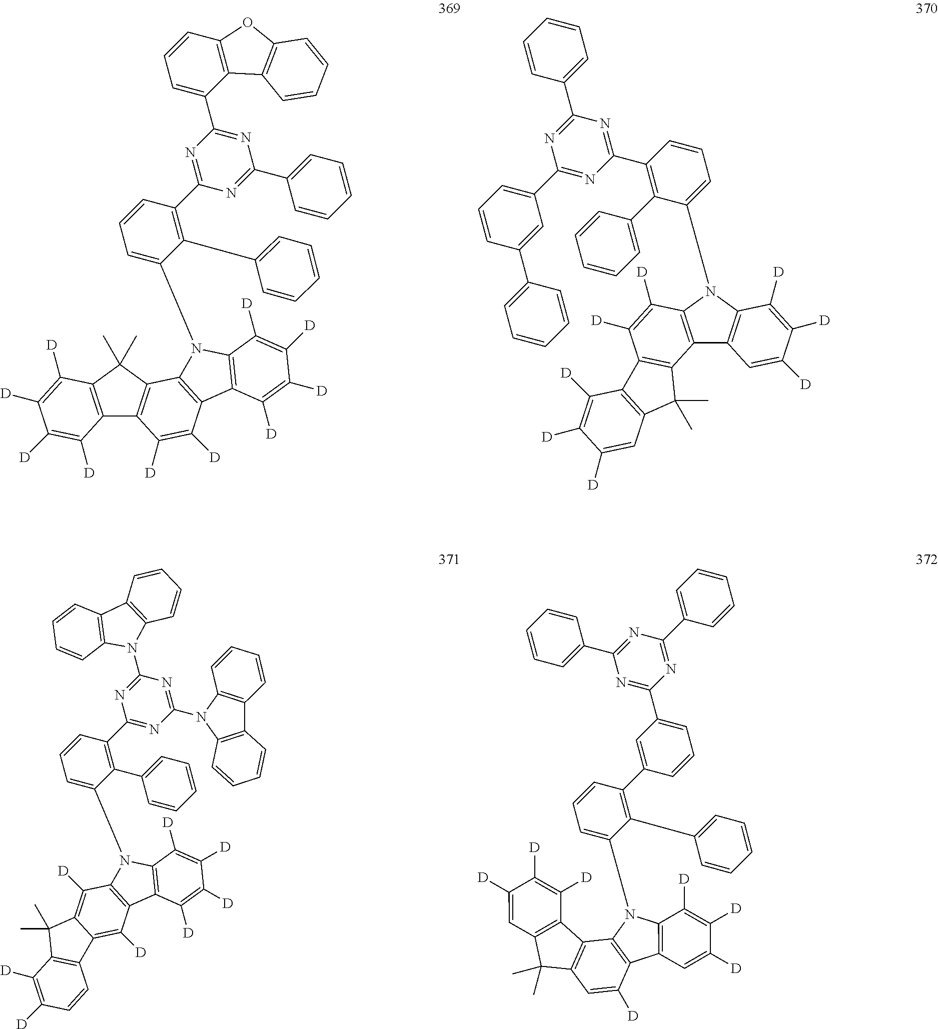

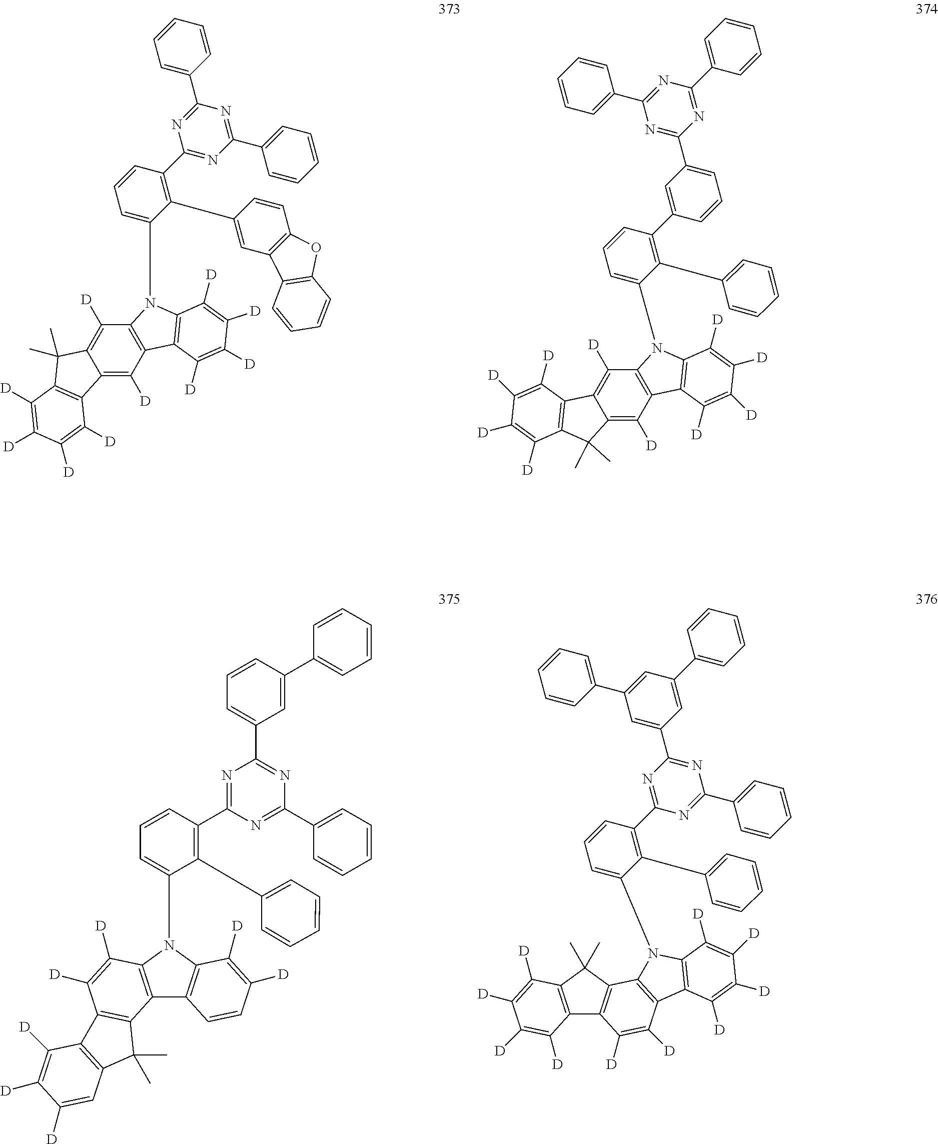

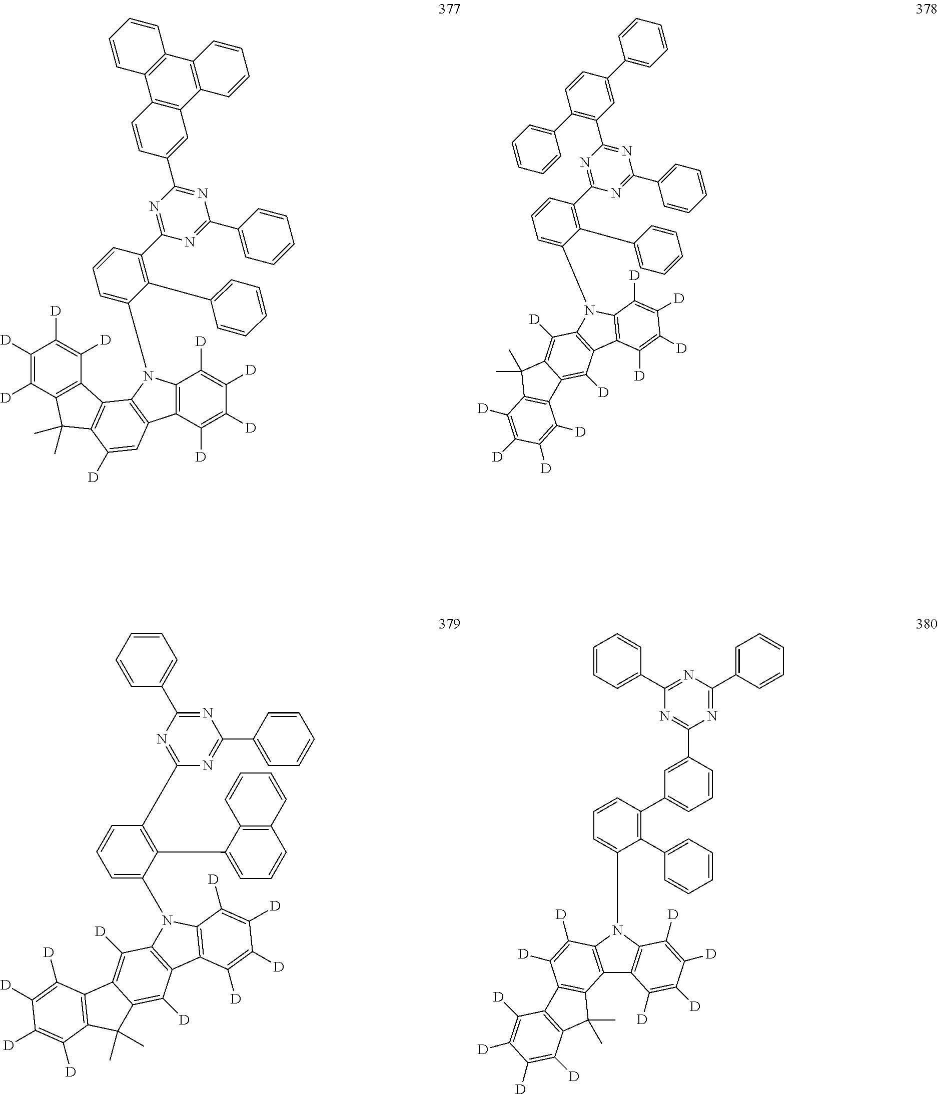

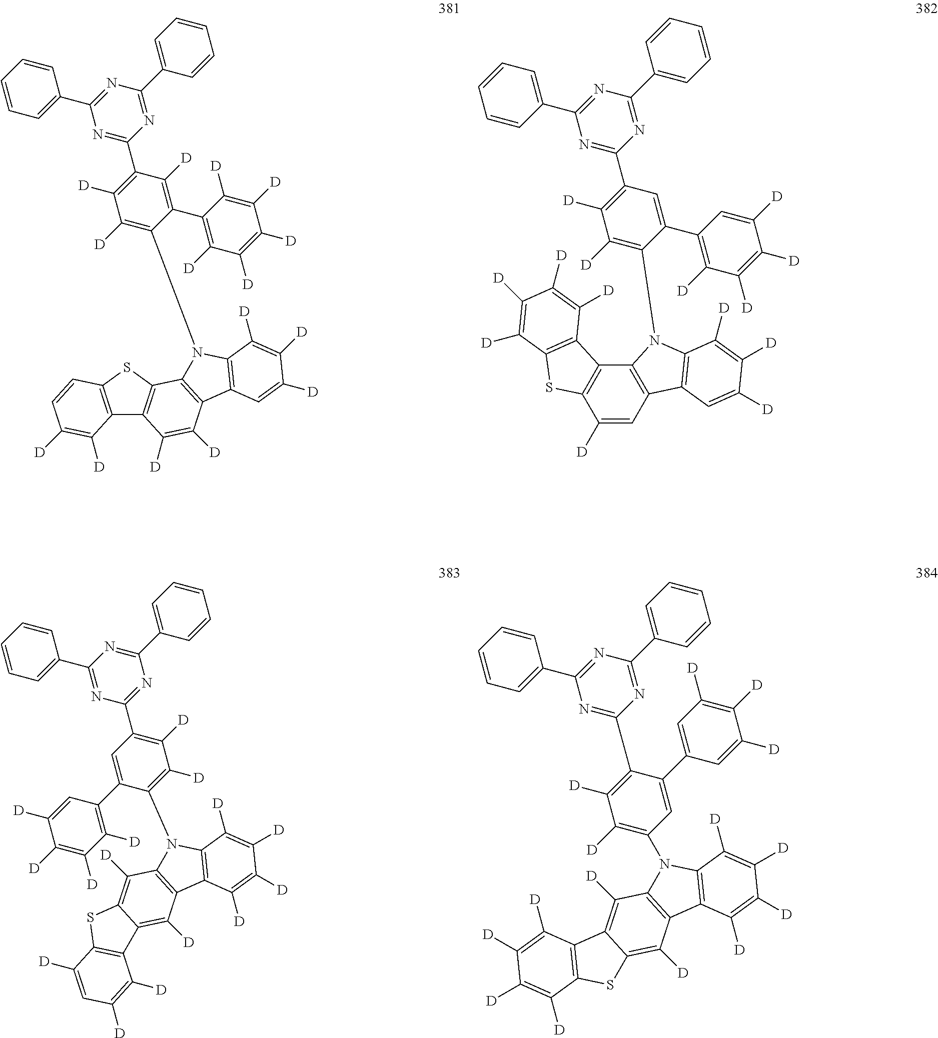

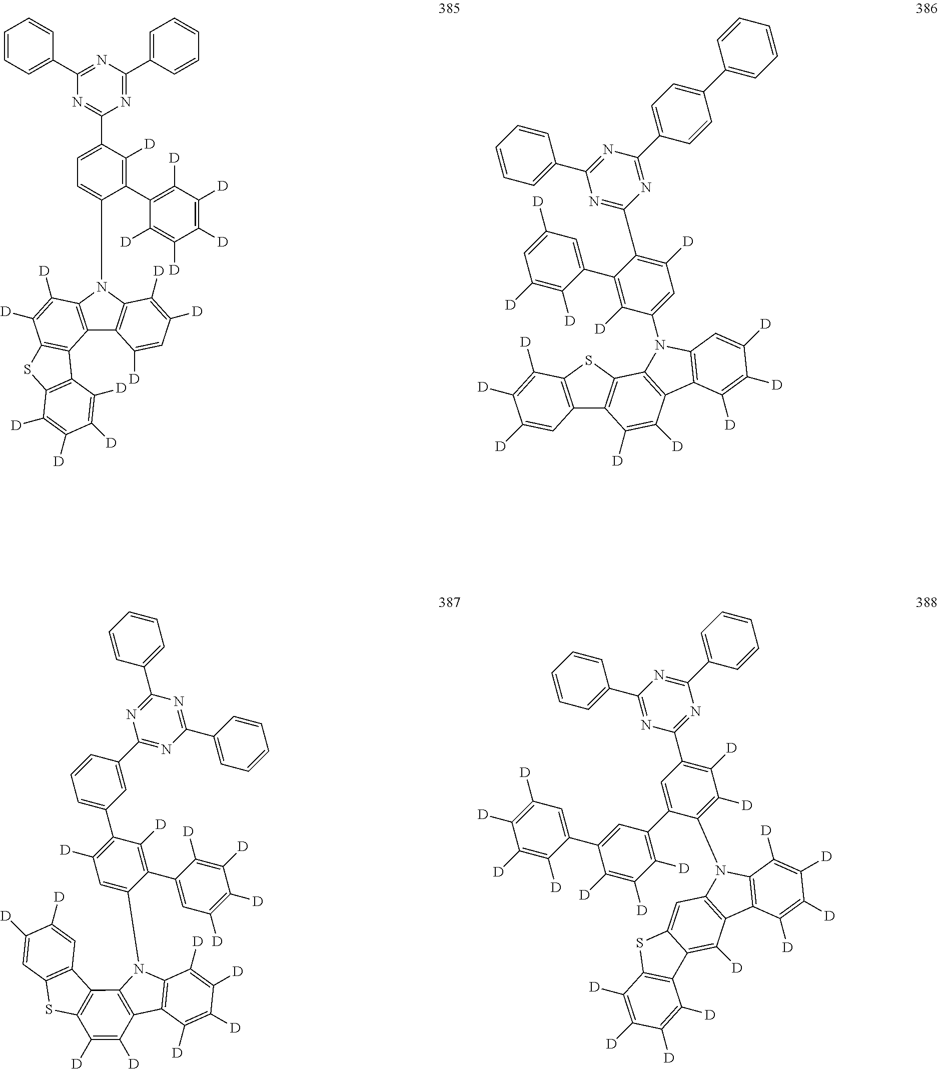

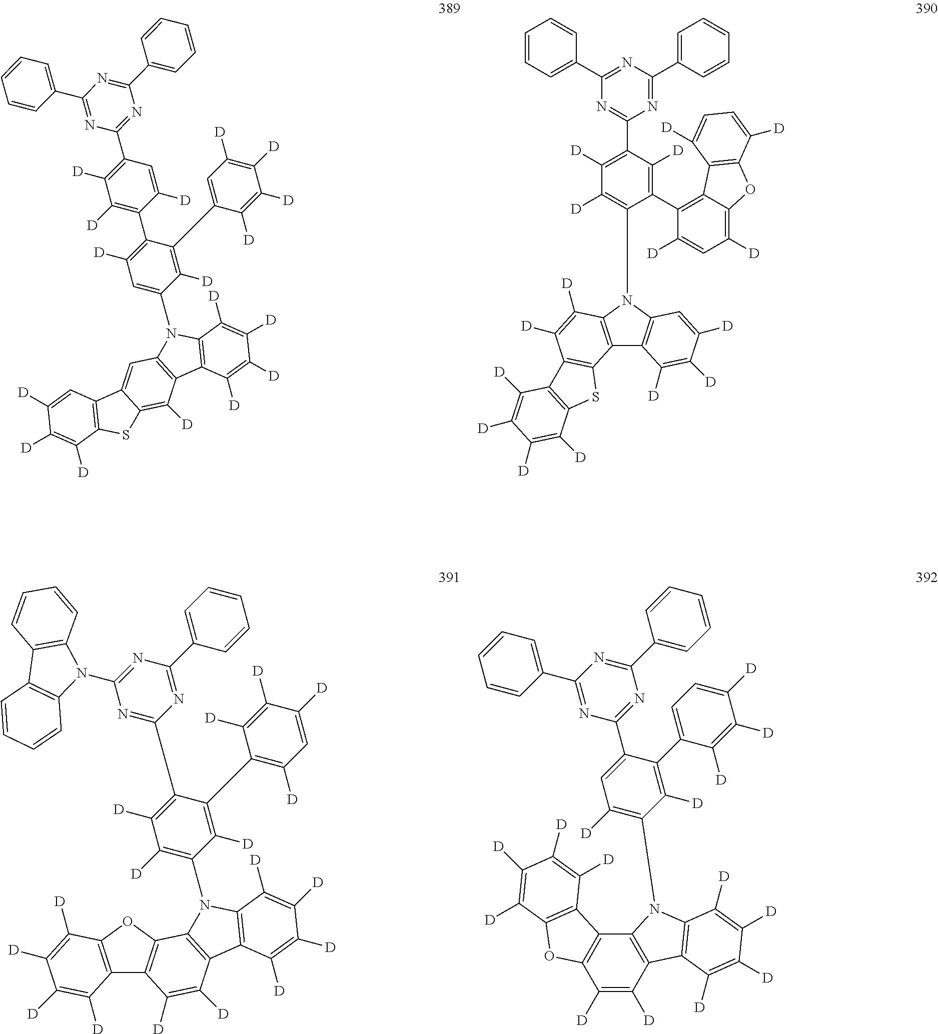

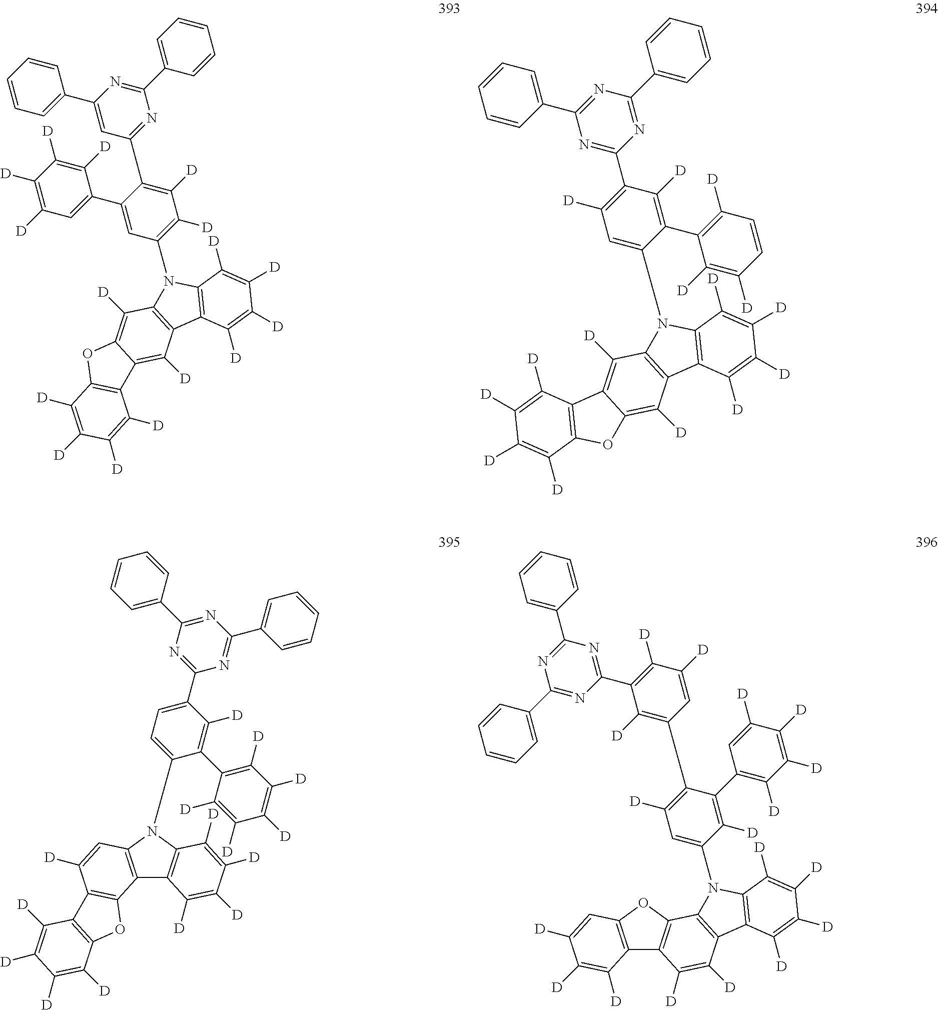

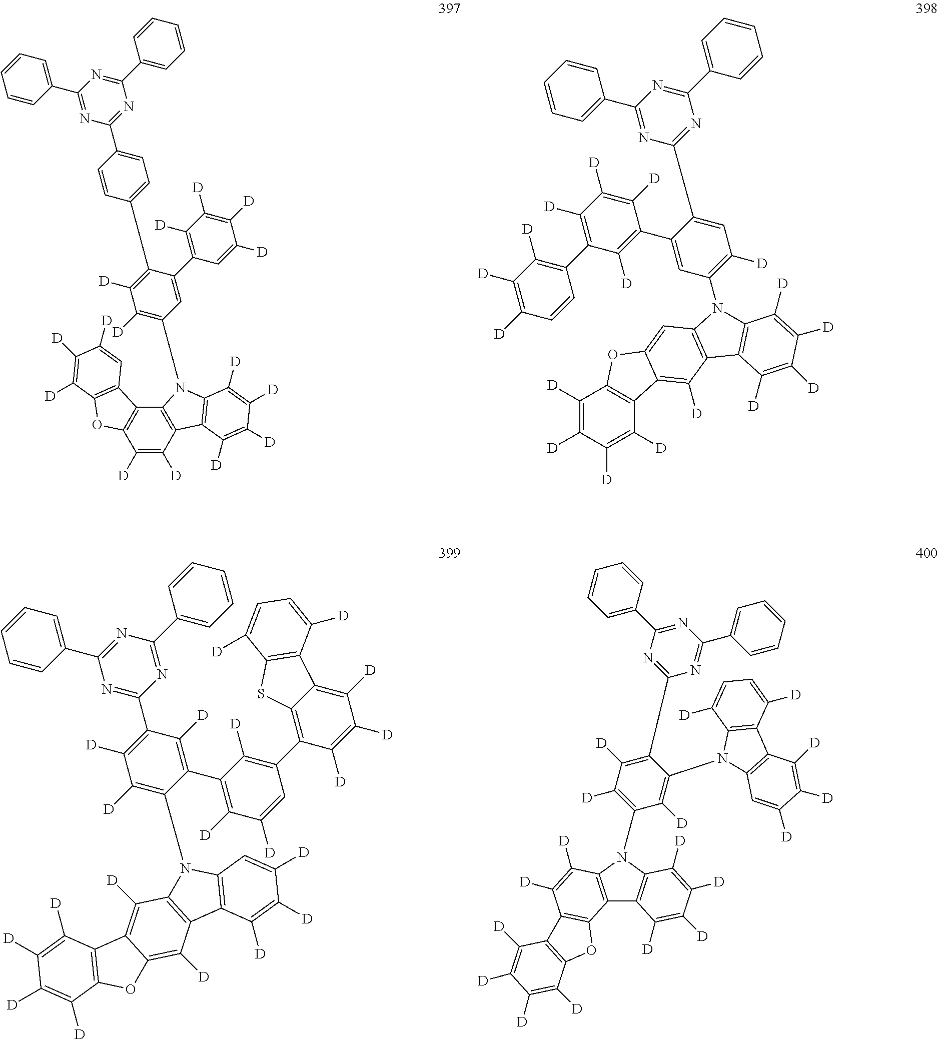

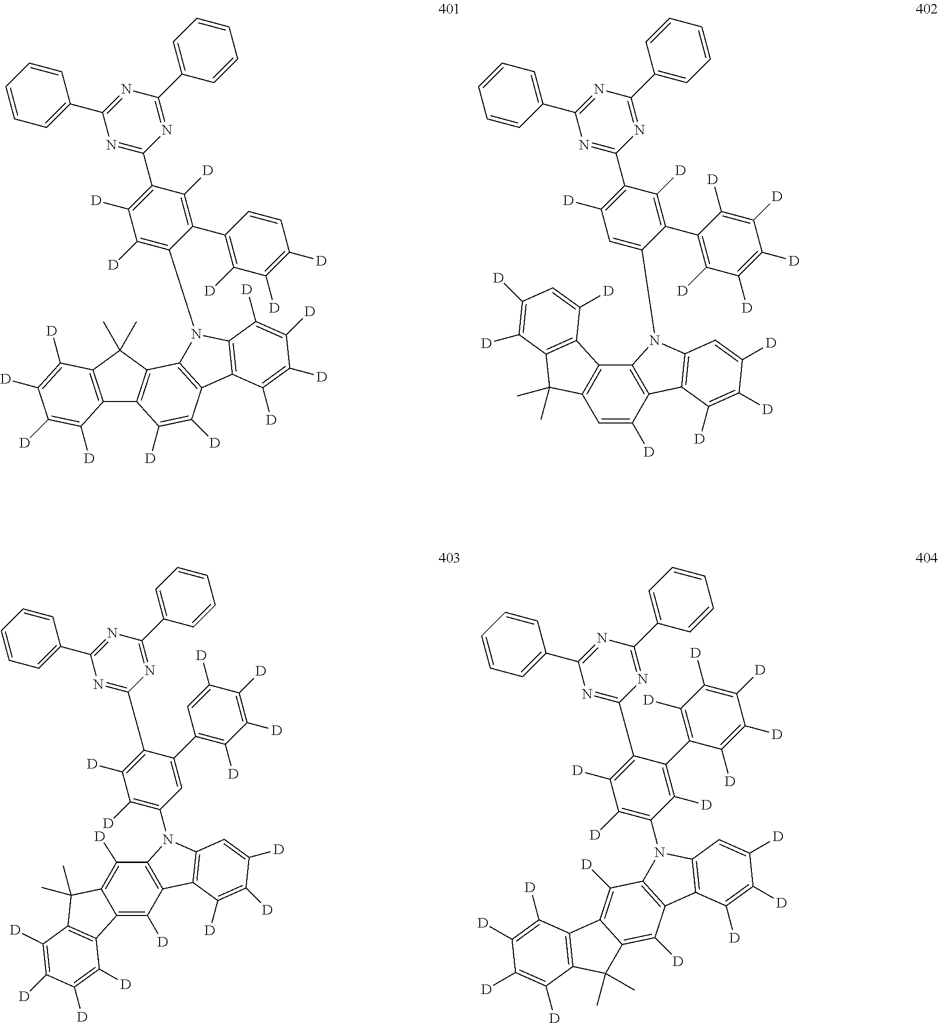

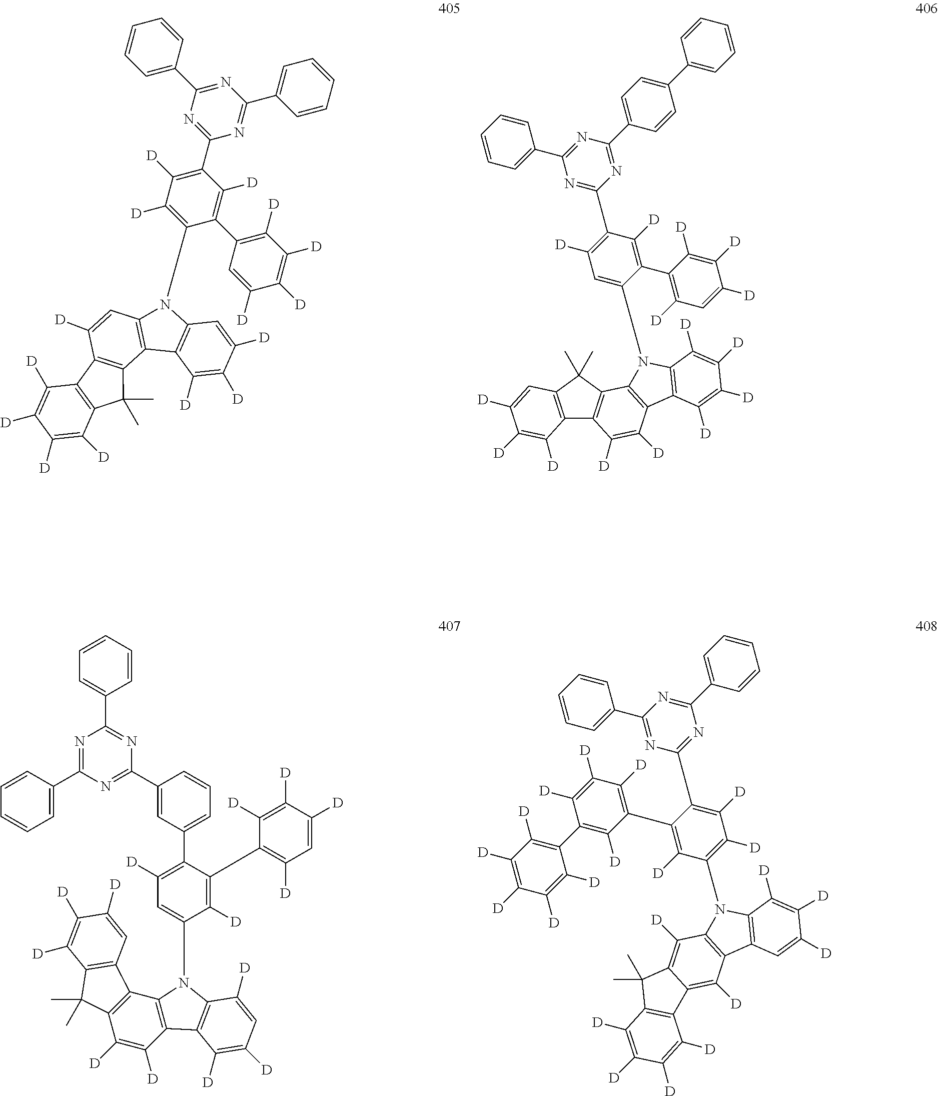

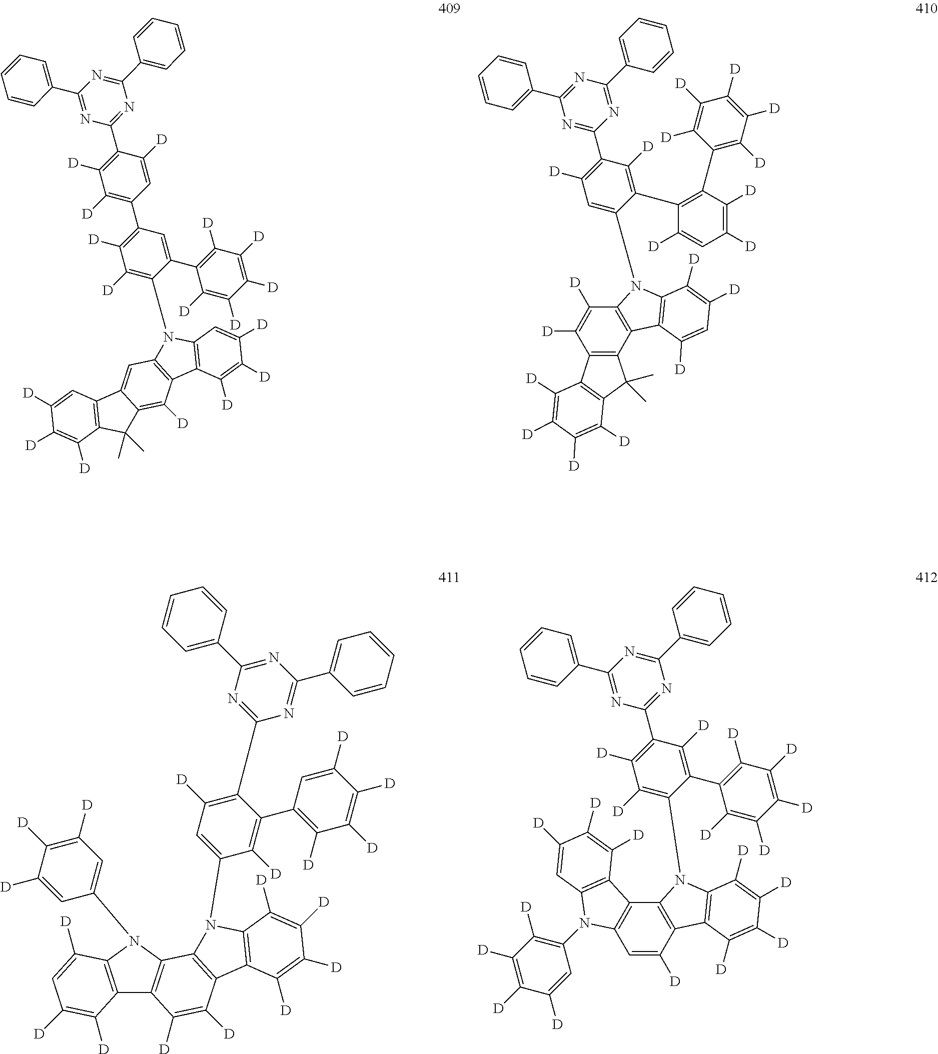

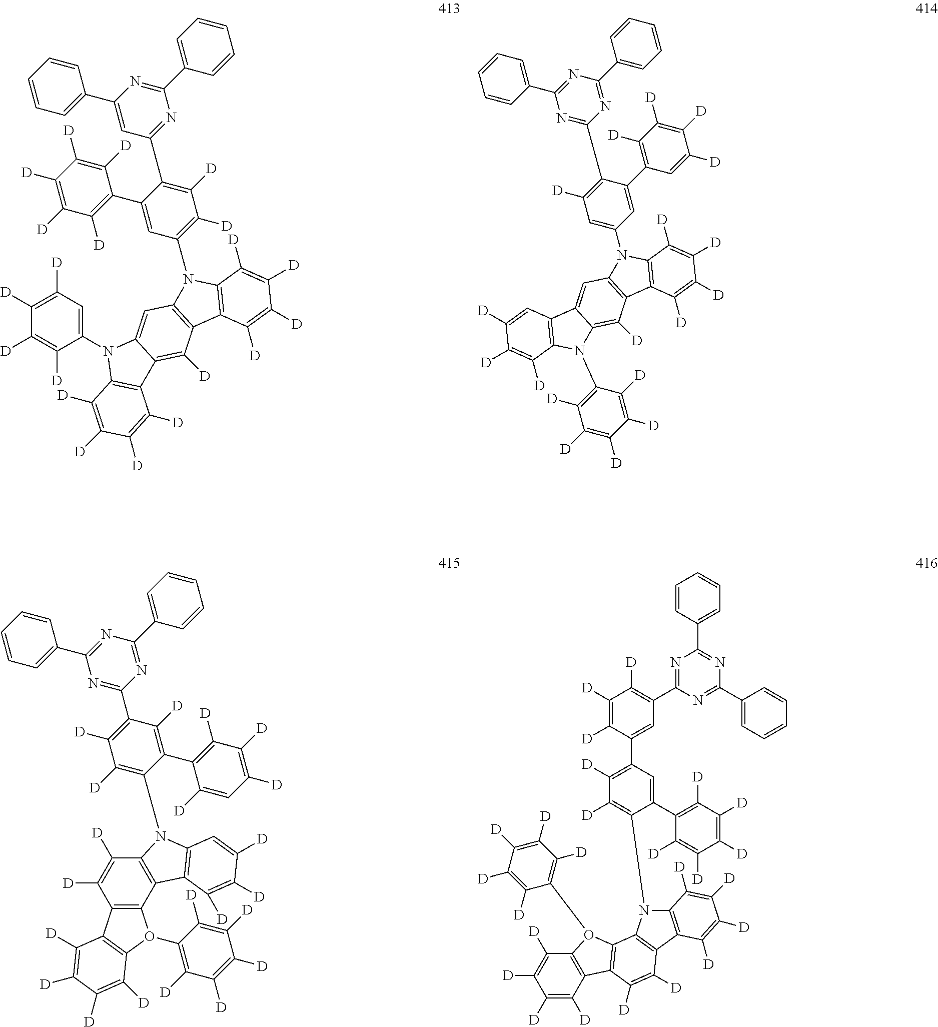

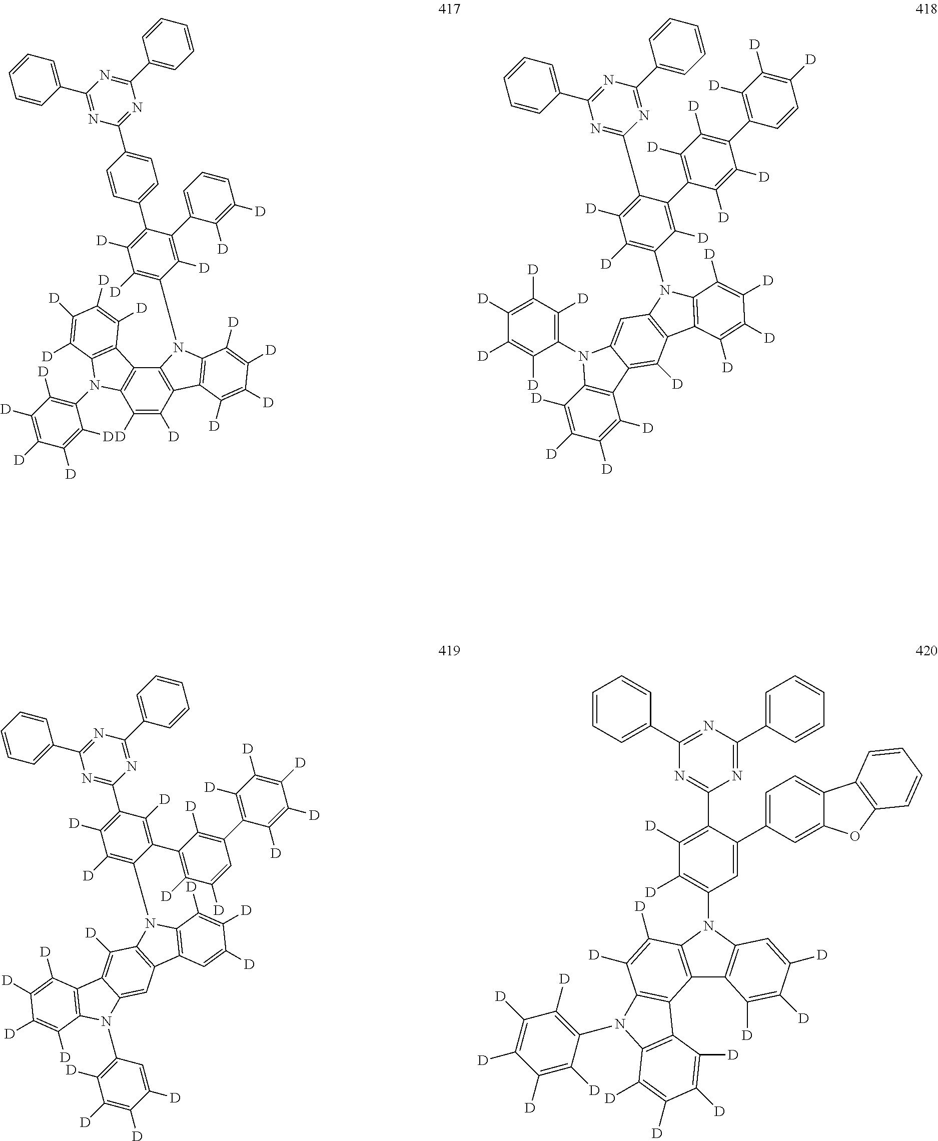

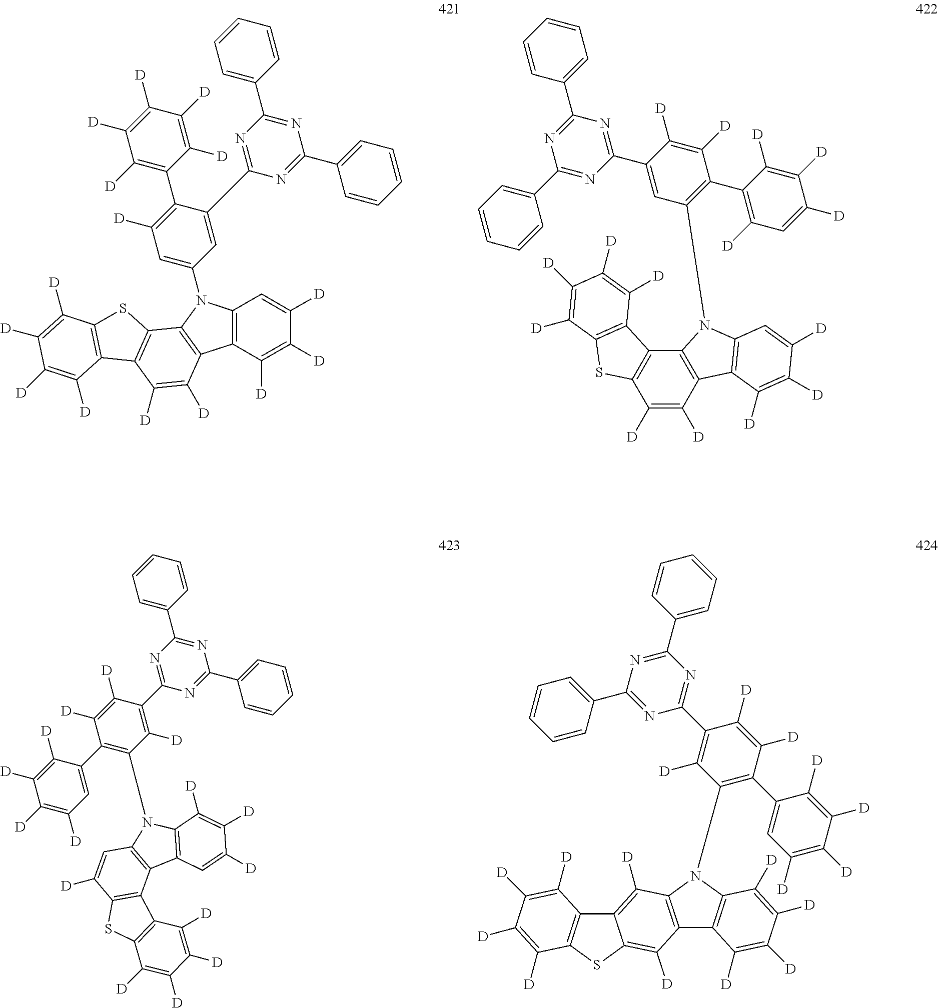

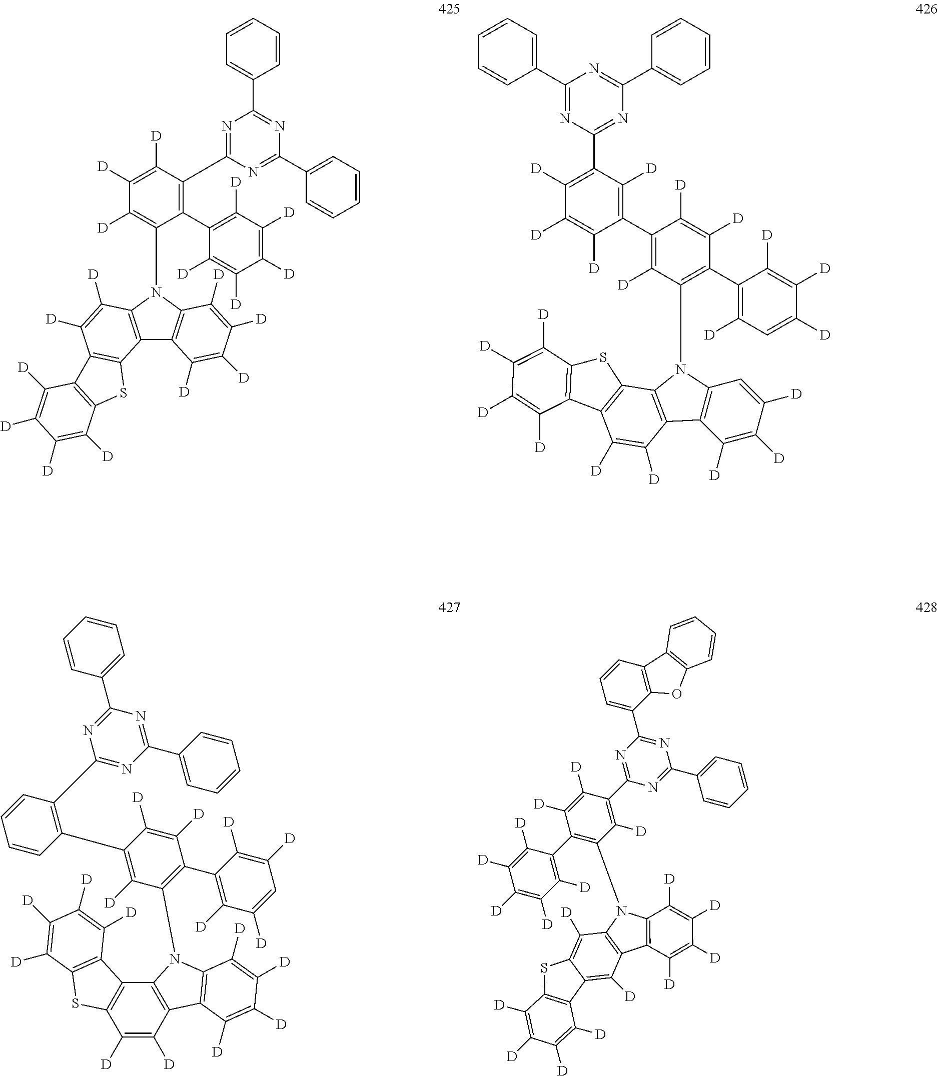

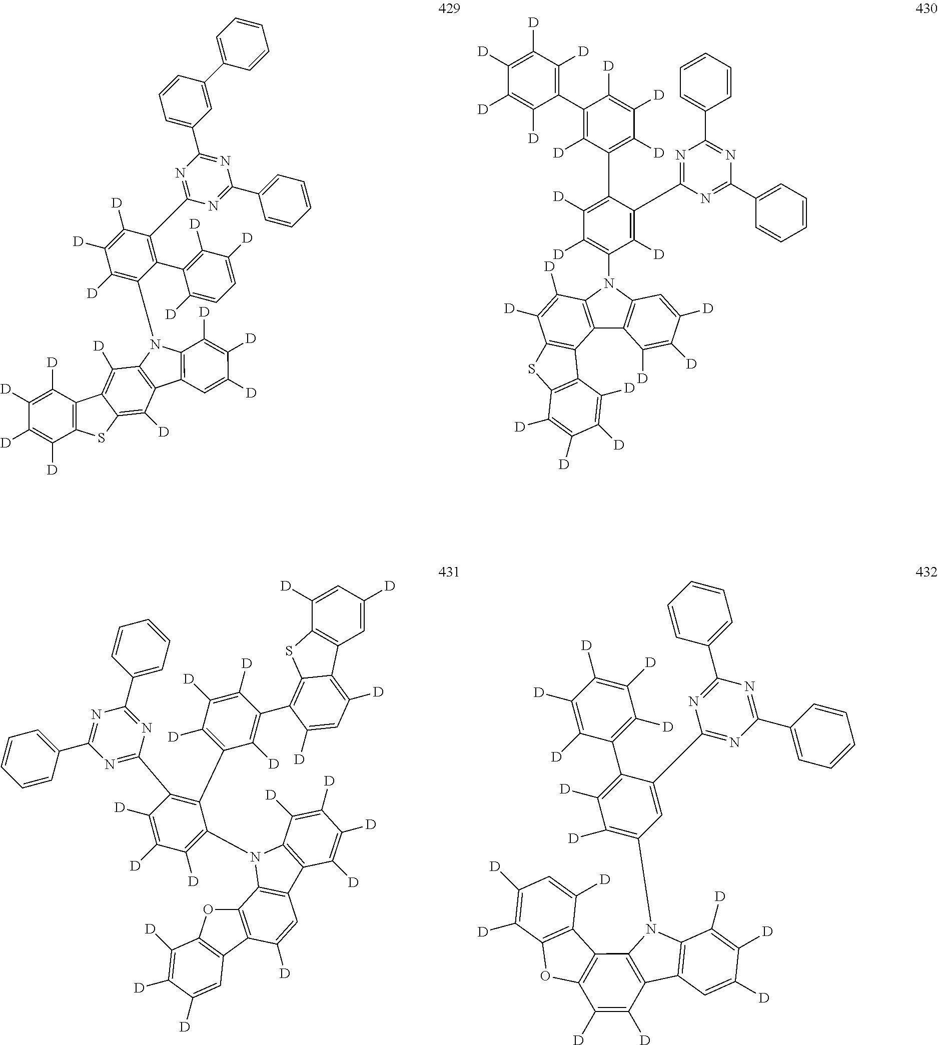

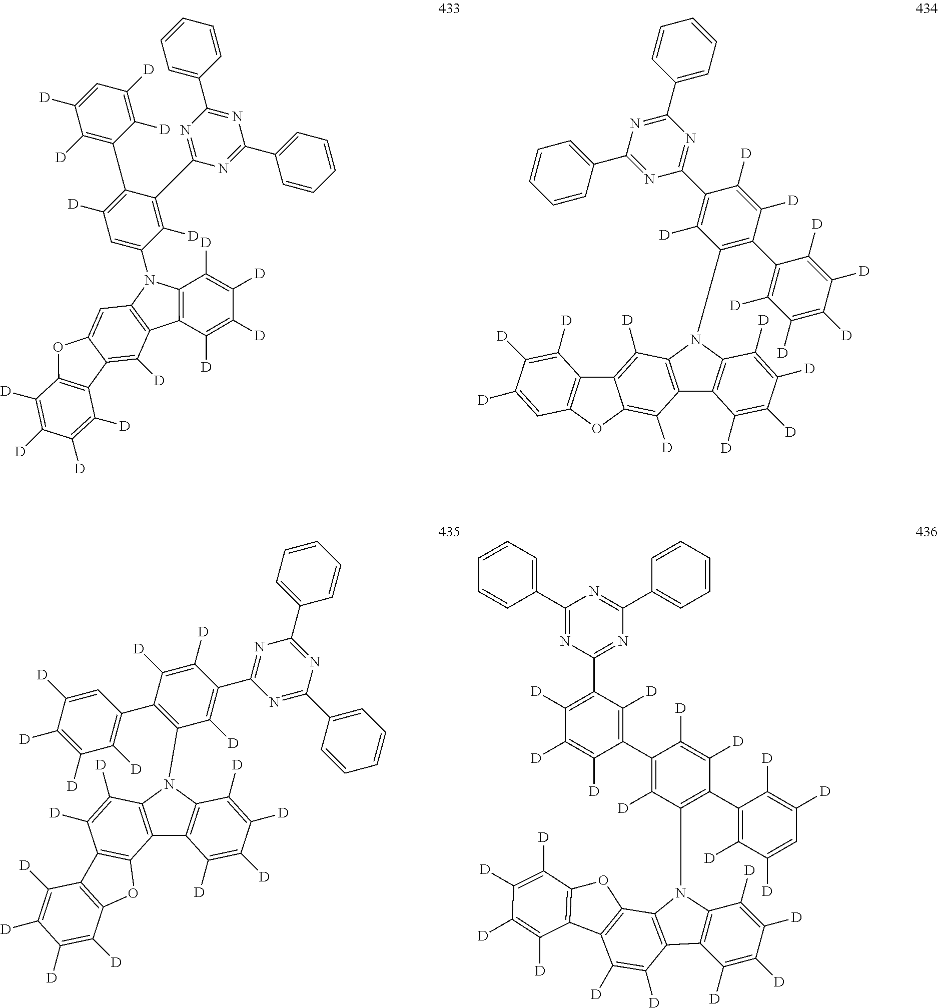

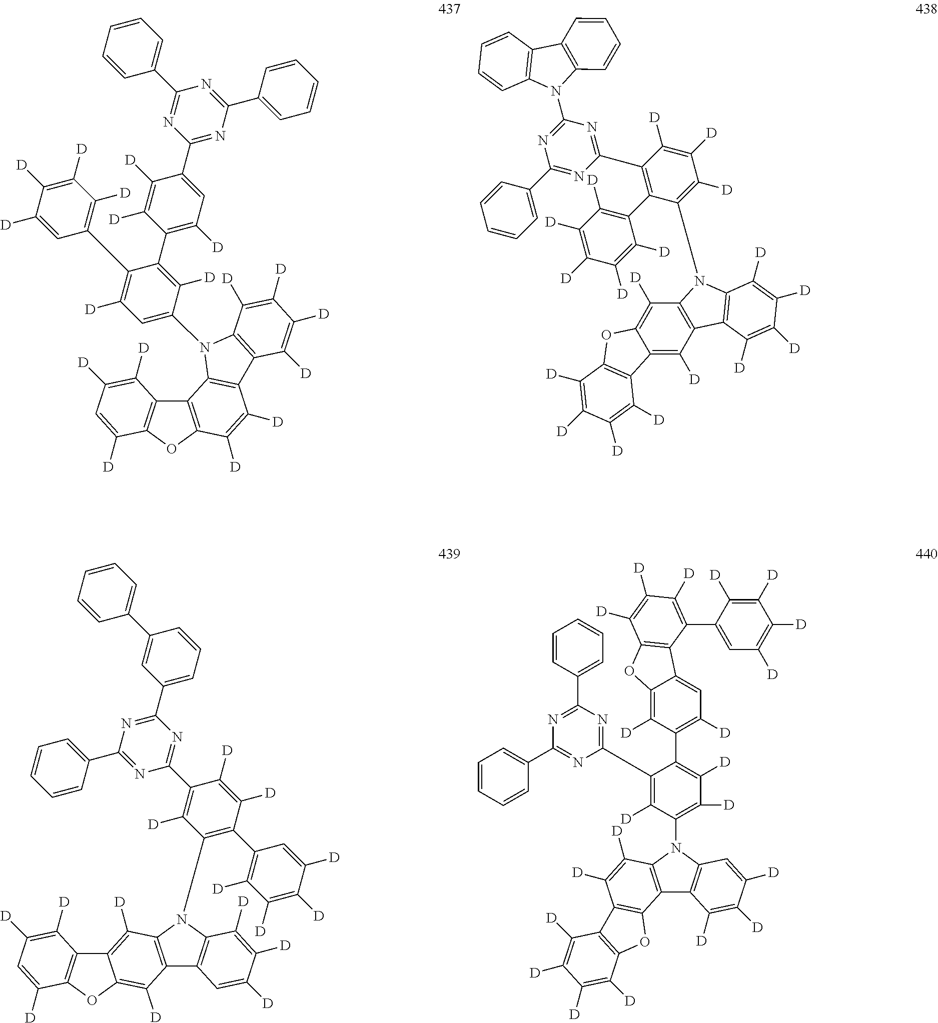

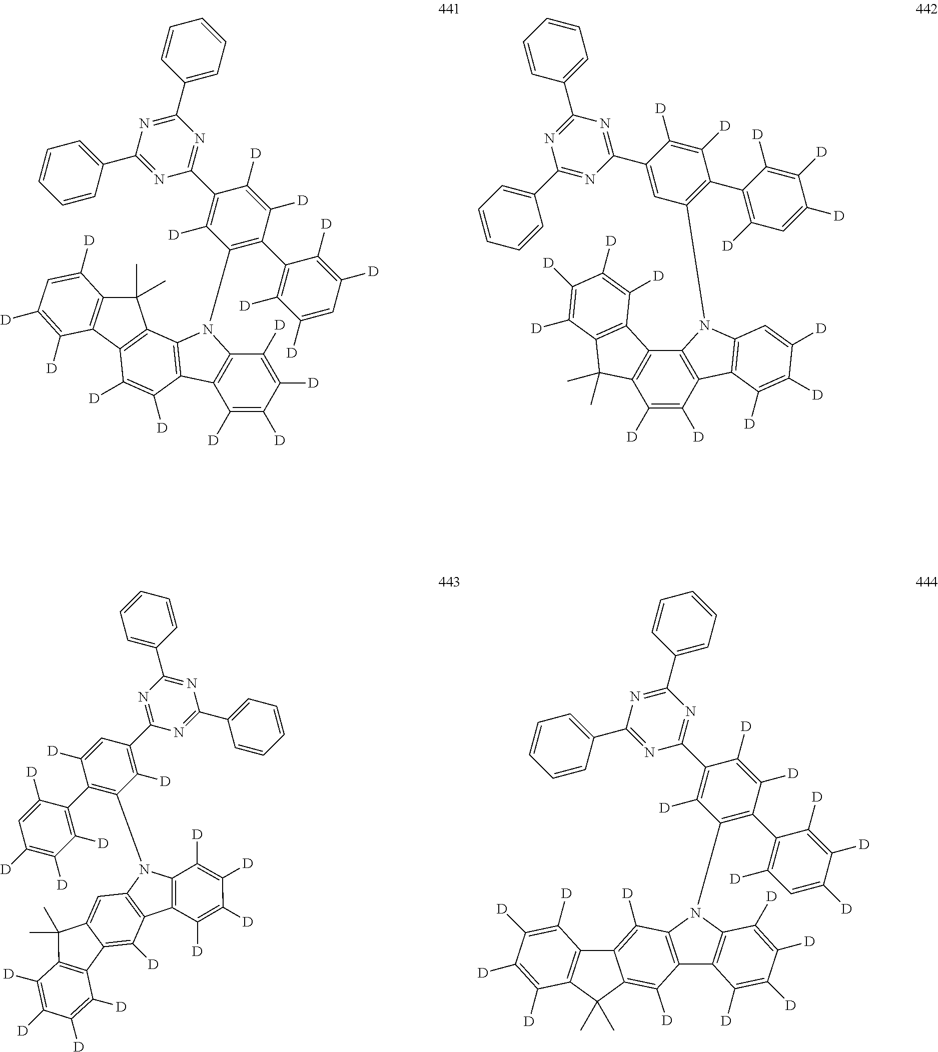

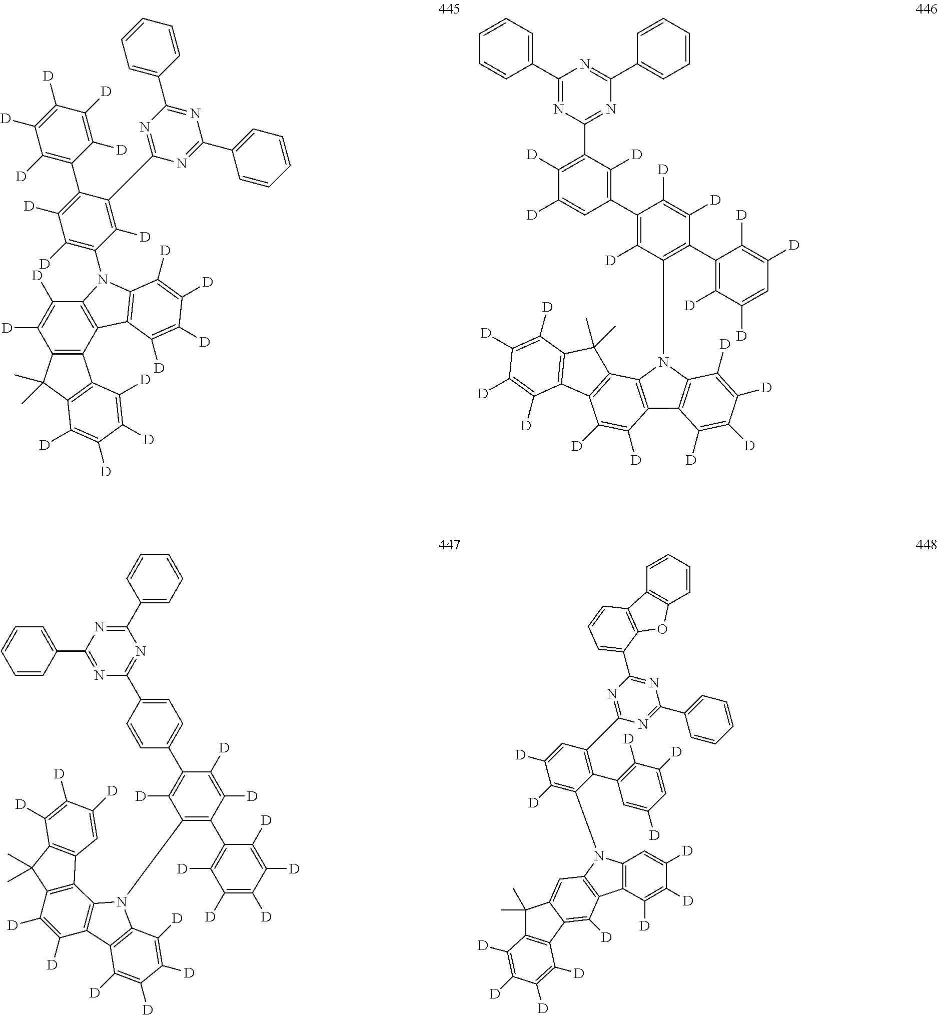

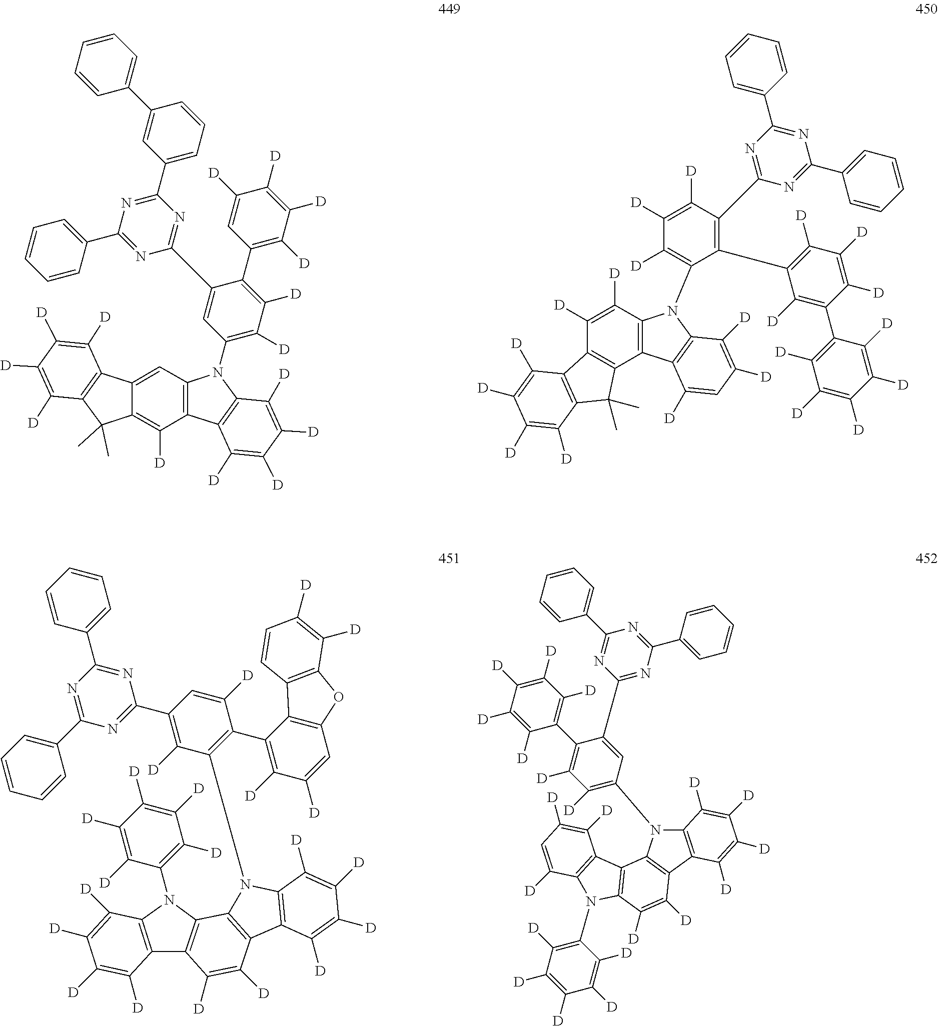

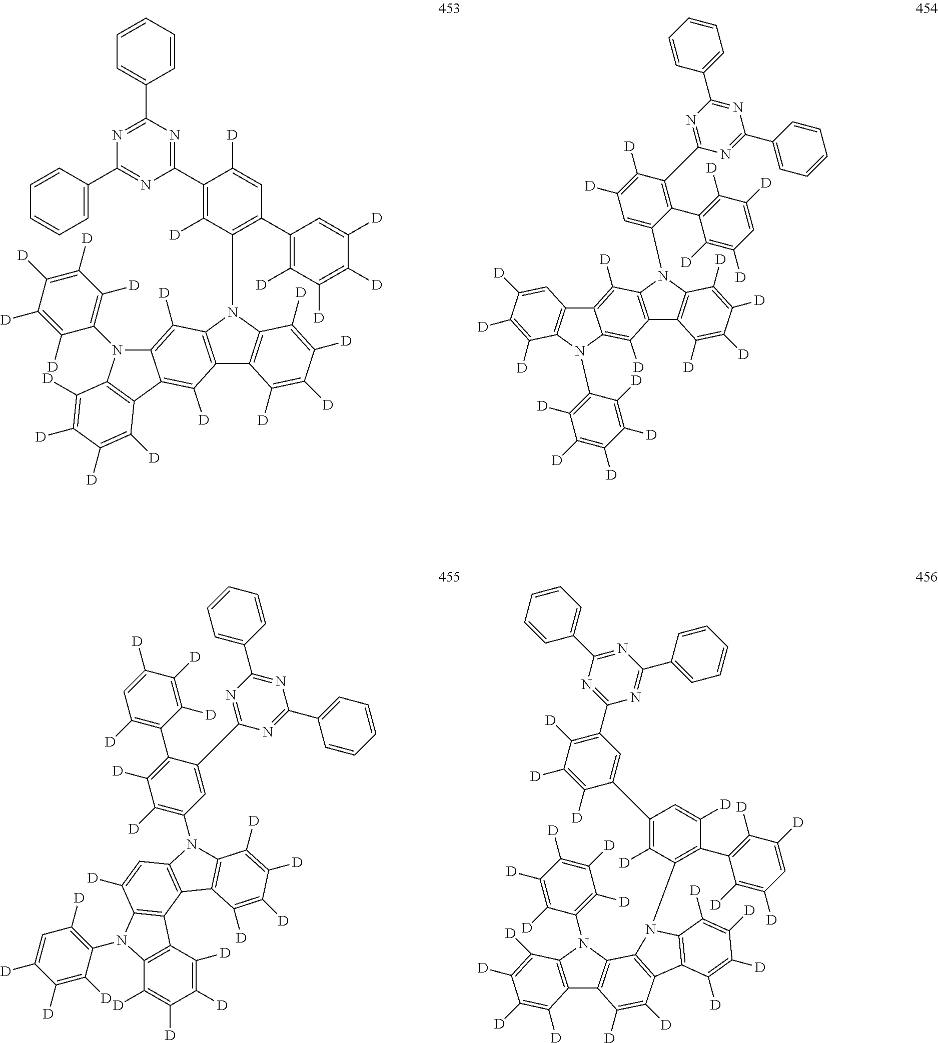

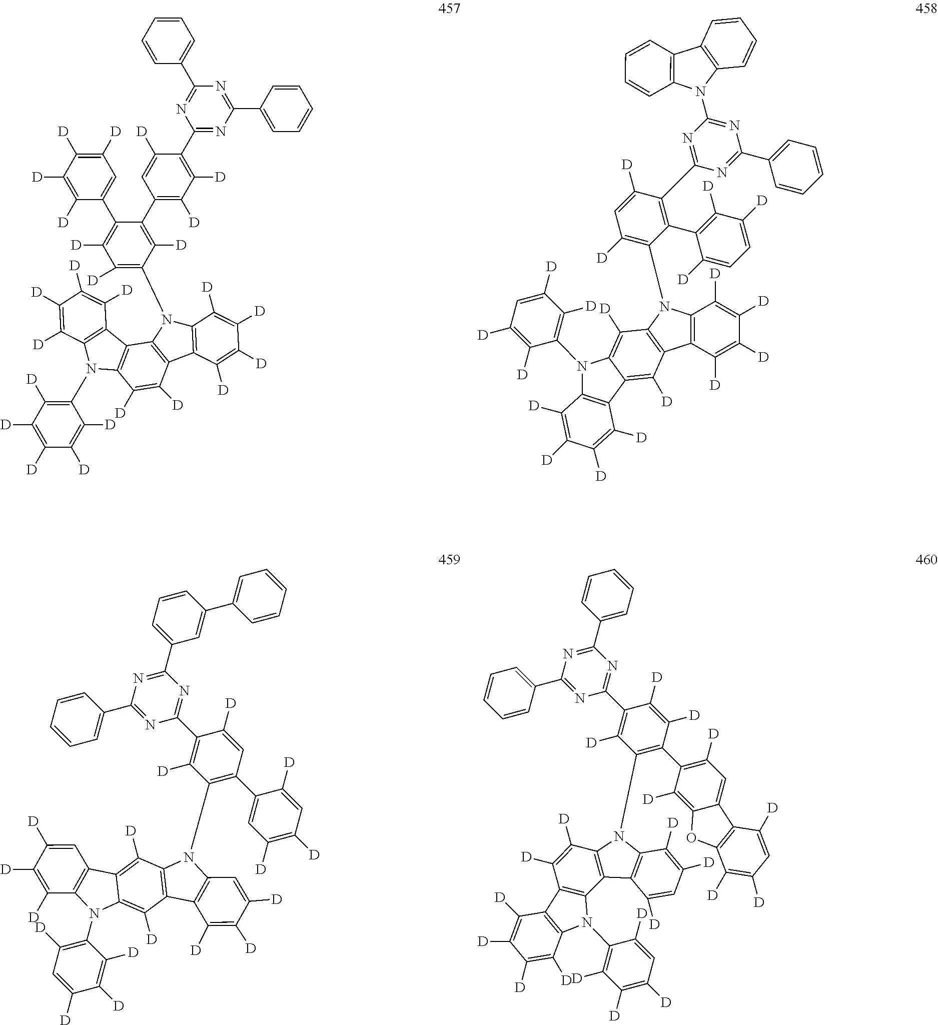

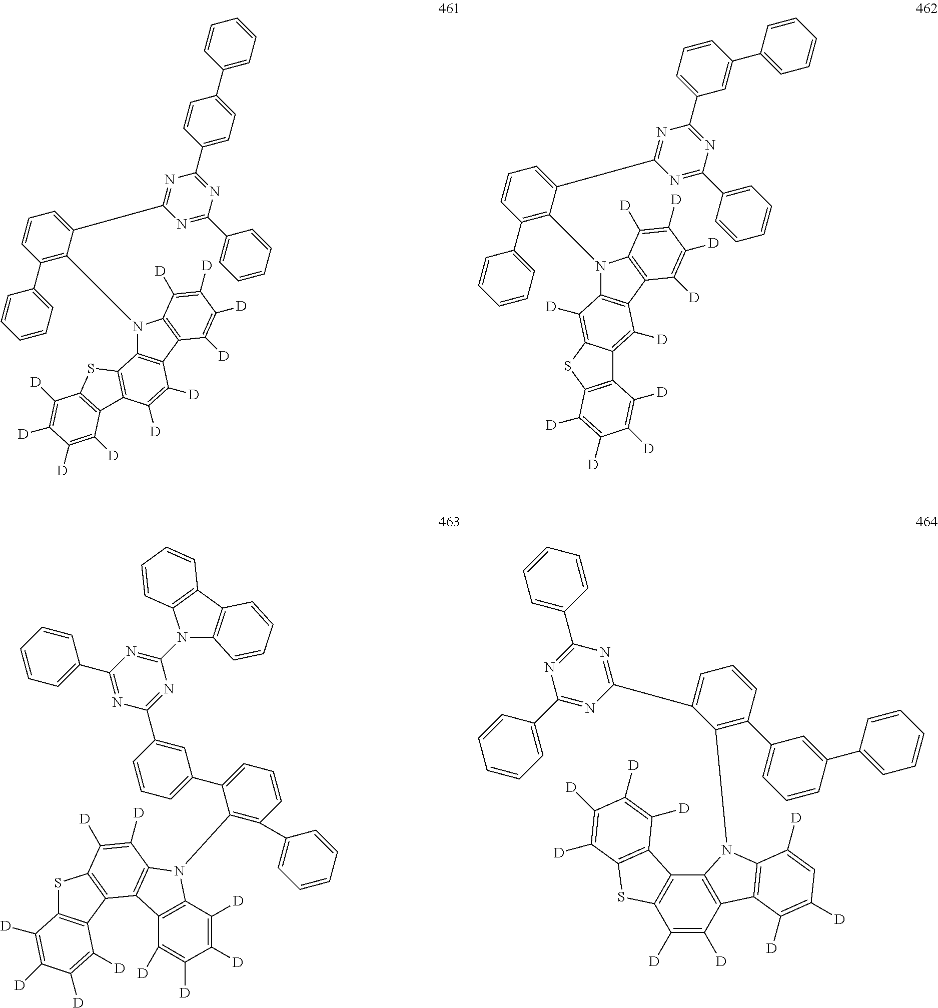

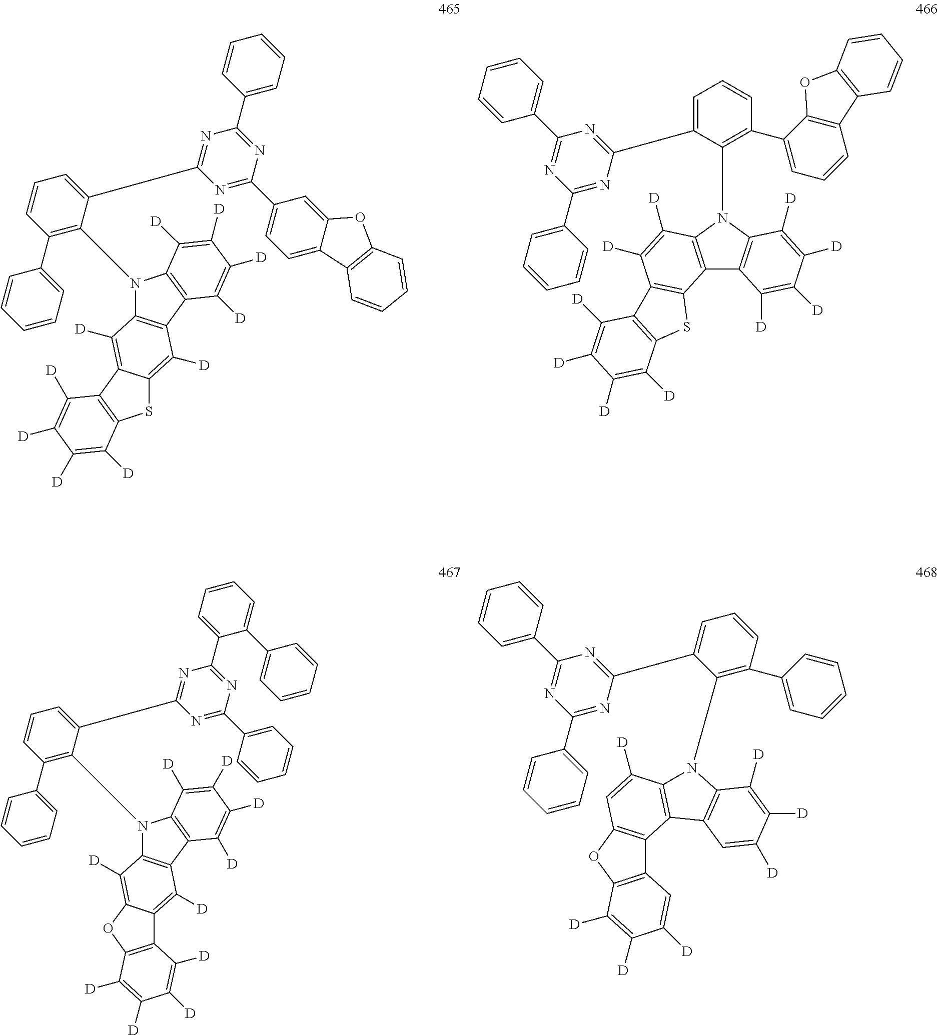

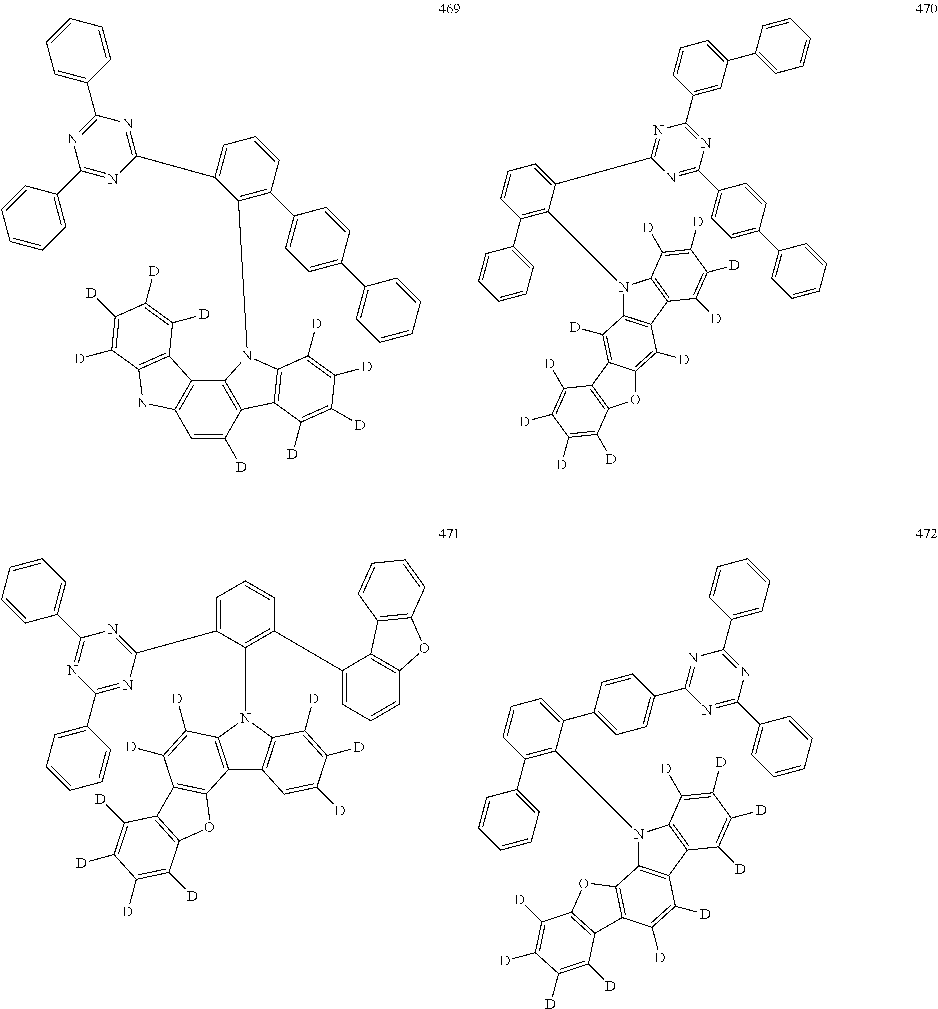

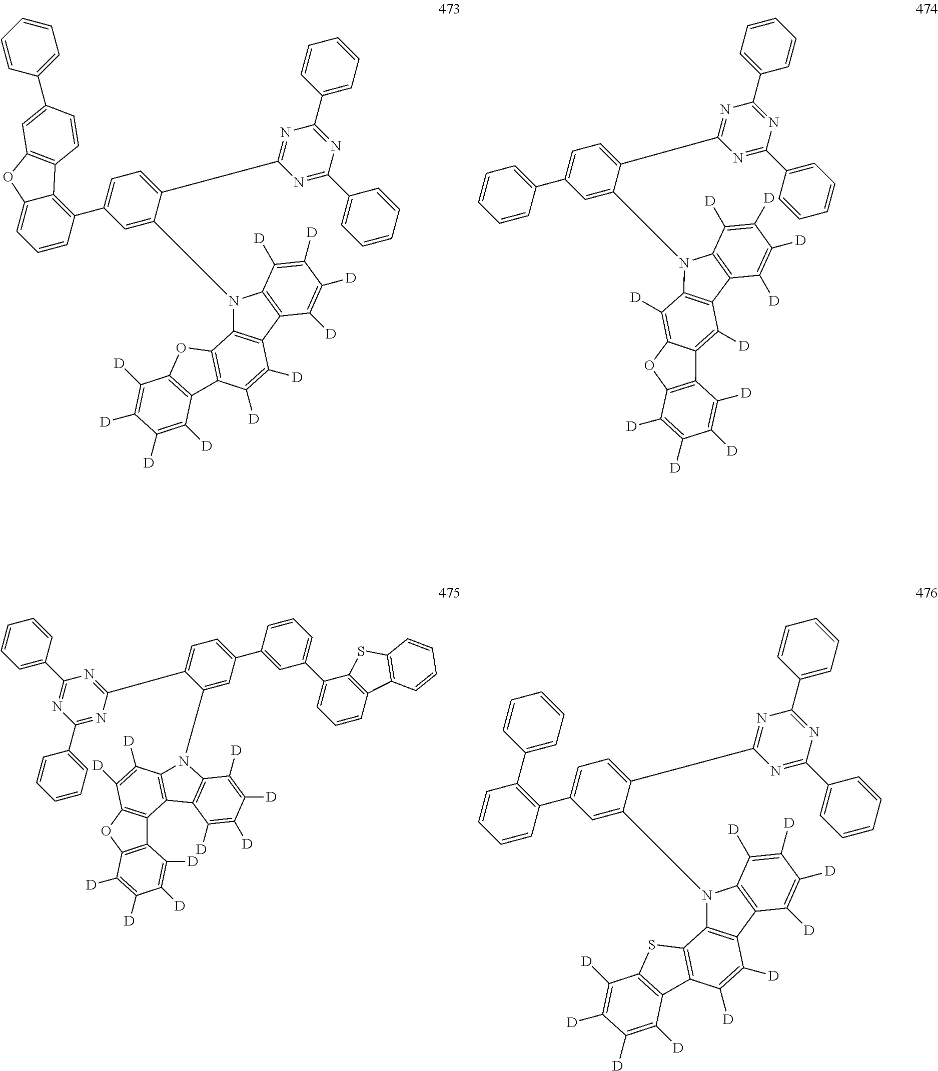

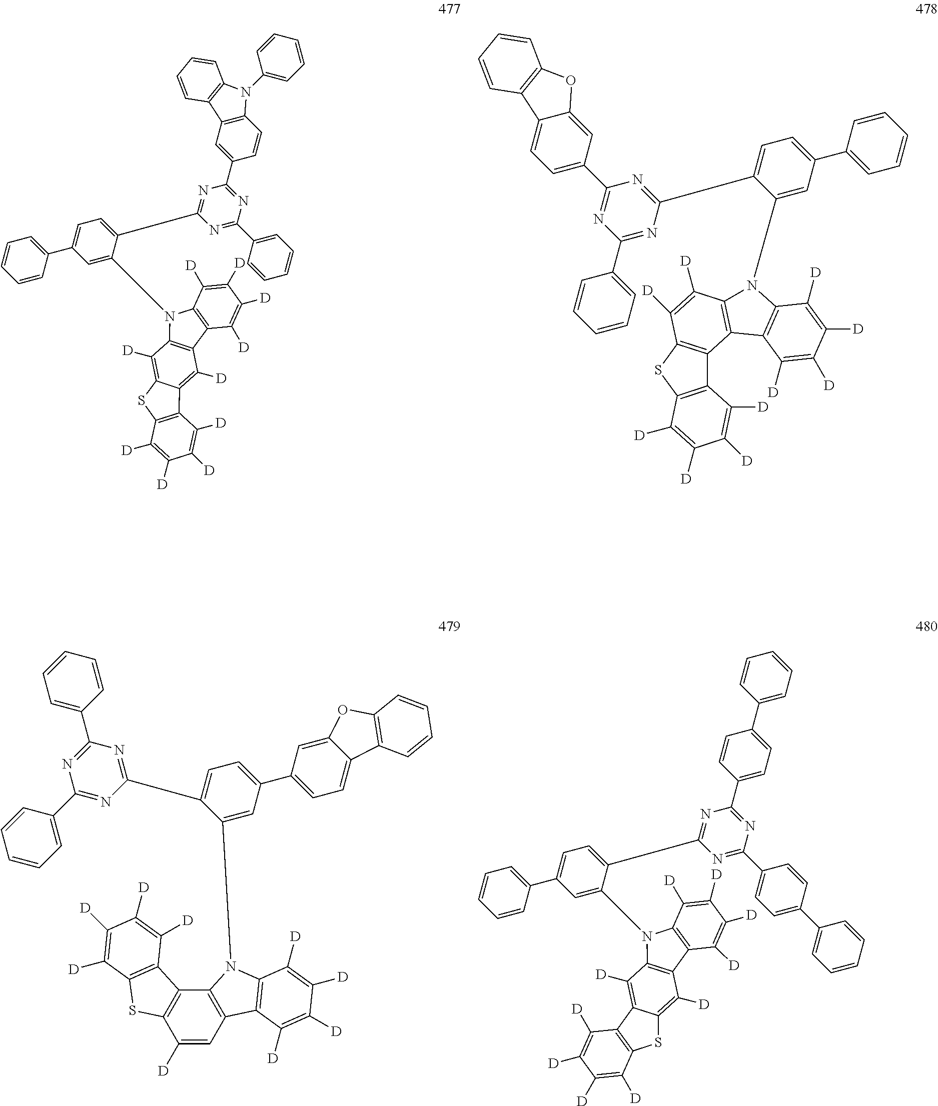

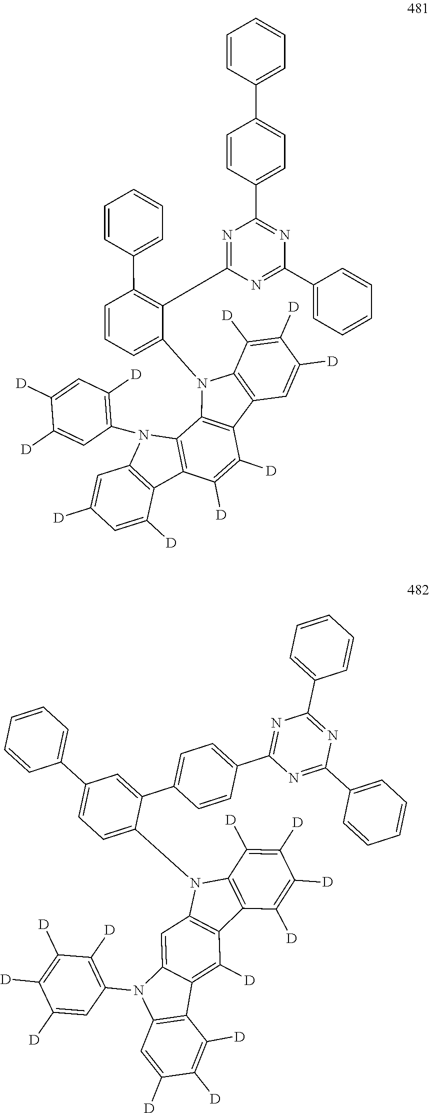 [SMILES: [2H]c1cc([2H])c2c3c([2H])c([2H])c4c5cc([2H])c([2H])c([2H])c5n(-c5cccc(-c6ccccc6)c5-c5nc(-c6ccccc6)nc(-c6ccc(-c7ccccc7)cc6)n5)c4c3n(-c3cc([2H])c([2H])cc3[2H])c2c1 ; [2H]c1cc(-n2c3cc([2H])c([2H])c([2H])c3c3c([2H])c4c5c([2H])c([2H])c([2H])c([2H])c5n(-c5ccc(-c6ccccc6)cc5-c5ccc(-c6nc(-c7ccccc7)nc(-c7ccccc7)n6)cc5)c4cc32)c([2H])c([2H])c1[2H]]

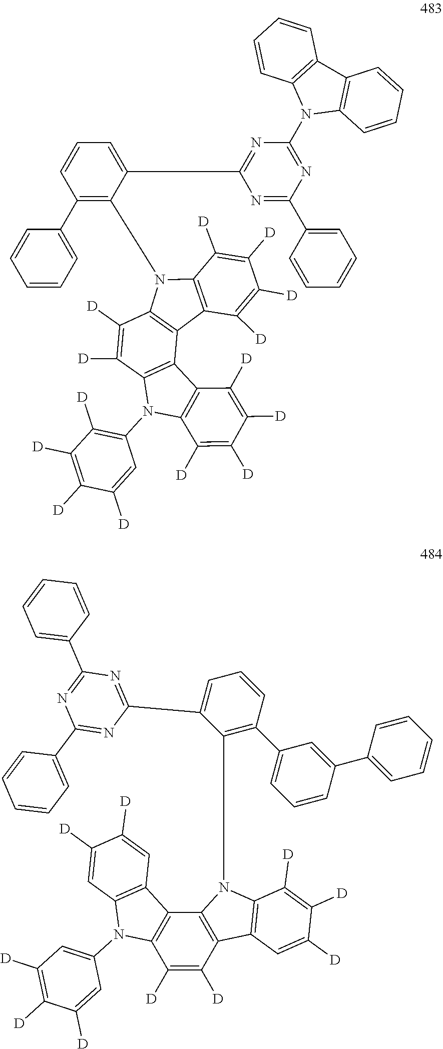 [SMILES: [2H]c1cc(-n2c3c([2H])c([2H])c([2H])c([2H])c3c3c4c5c([2H])c([2H])c([2H])c([2H])c5n(-c5c(-c6ccccc6)cccc5-c5nc(-c6ccccc6)nc(-n6c7ccccc7c7ccccc76)n5)c4c([2H])c([2H])c32)c([2H])c([2H])c1[2H] ; [2H]c1cc2c3c(c([2H])c([2H])c4c5cc([2H])c([2H])c([2H])c5n(-c5c(-c6cccc(-c7ccccc7)c6)cccc5-c5nc(-c6ccccc6)nc(-c6ccccc6)n5)c43)n(-c3cc([2H])c([2H])c([2H])c3)c2cc1[2H]]

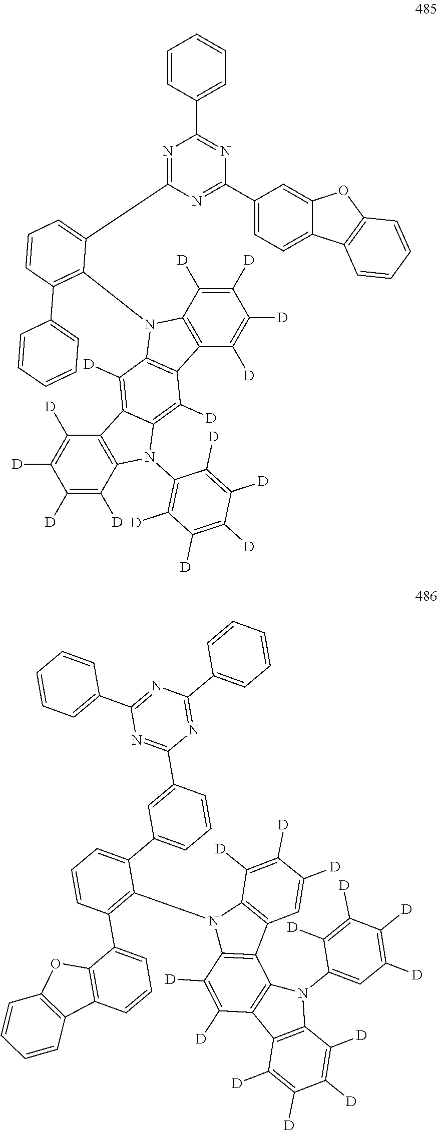 [SMILES: [2H]c1c([2H])c([2H])c(-n2c3c([2H])c([2H])c([2H])c([2H])c3c3c([2H])c4c(c([2H])c32)c2c([2H])c([2H])c([2H])c([2H])c2n4-c2c(-c3ccccc3)cccc2-c2nc(-c3ccccc3)nc(-c3ccc4c(c3)oc3ccccc34)n2)c([2H])c1[2H] ; [2H]c1cc(-n2c3c([2H])c([2H])c([2H])c([2H])c3c3c([2H])c([2H])c4c(c5cc([2H])c([2H])c([2H])c5n4-c4c(-c5cccc(-c6nc(-c7ccccc7)nc(-c7ccccc7)n6)c5)cccc4-c4cccc5c4oc4ccccc45)c32)c([2H])c([2H])c1[2H]]

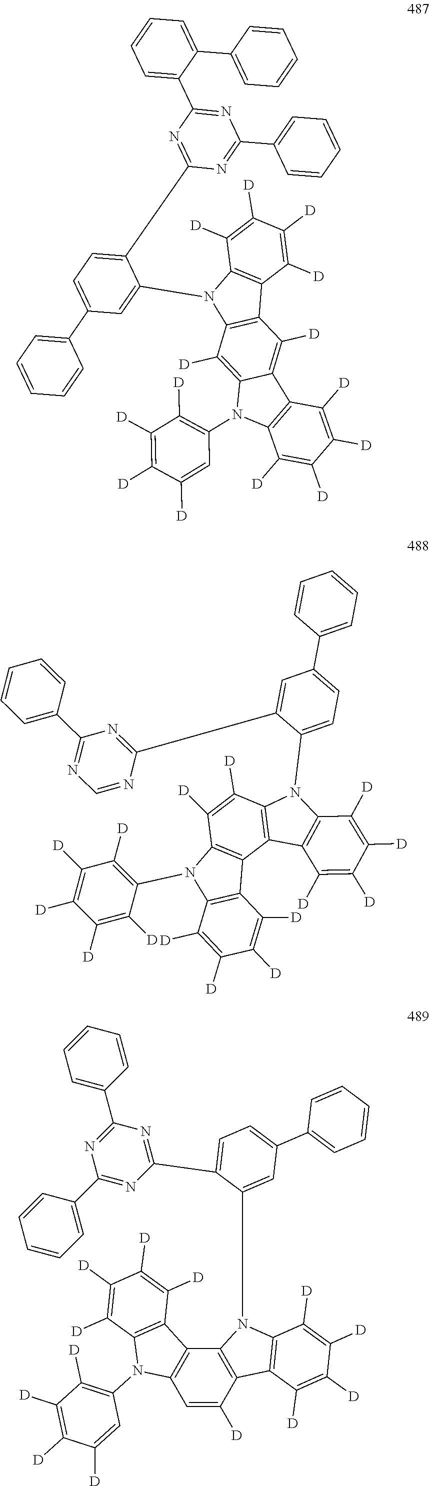

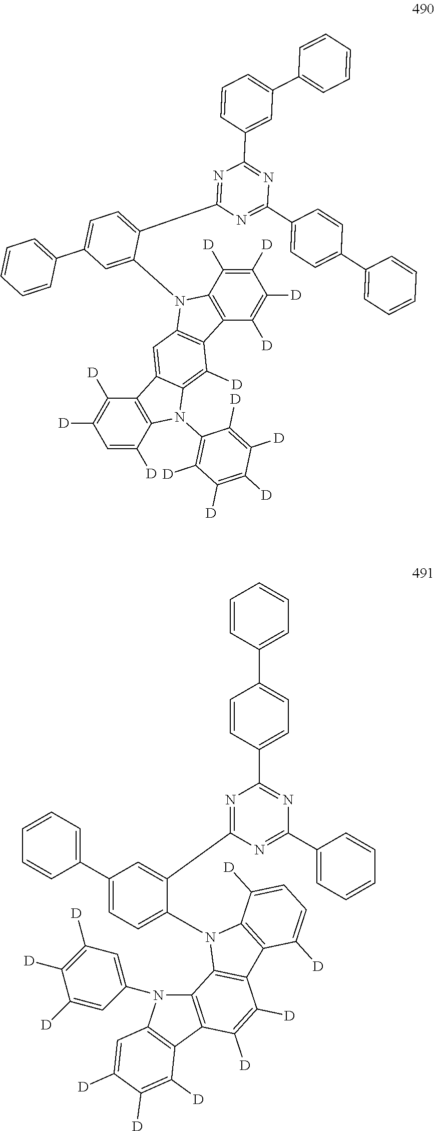 [SMILES: [2H]c1cc([2H])c2c(c1[2H])c1cc3c(c([2H])c1n2-c1c([2H])c([2H])c([2H])c([2H])c1[2H])c1c([2H])c([2H])c([2H])c([2H])c1n3-c1cc(-c2ccccc2)ccc1-c1nc(-c2ccc(-c3ccccc3)cc2)nc(-c2cccc(-c3ccccc3)c2)n1 ; [2H]c1cc(-n2c3cc([2H])c([2H])c([2H])c3c3c([2H])c([2H])c4c5c([2H])ccc([2H])c5n(-c5ccc(-c6ccccc6)cc5-c5nc(-c6ccccc6)nc(-c6ccc(-c7ccccc7)cc6)n5)c4c32)cc([2H])c1[2H]]

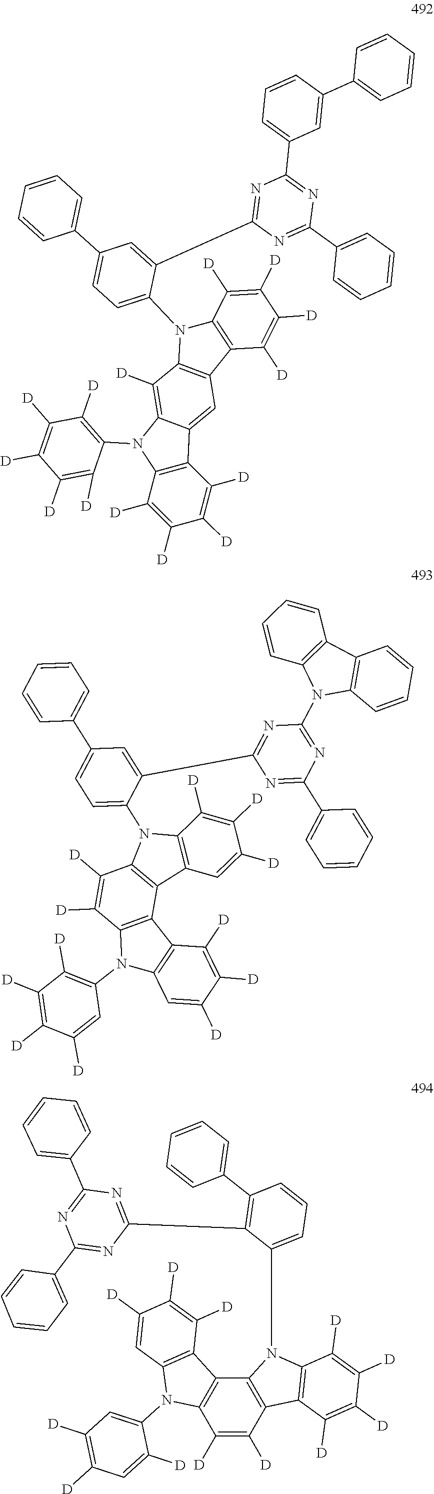

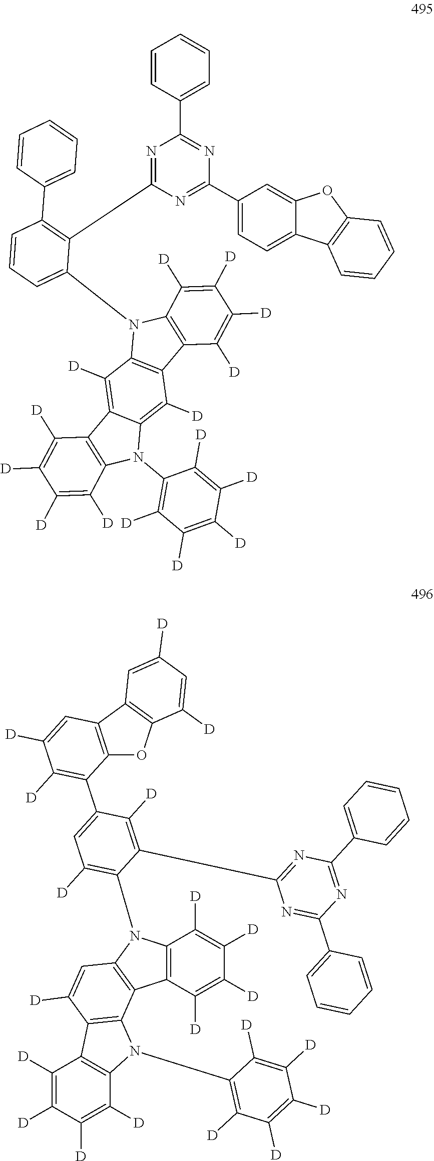 [SMILES: [2H]c1c([2H])c([2H])c(-n2c3c([2H])c([2H])c([2H])c([2H])c3c3c([2H])c4c(c([2H])c32)c2c([2H])c([2H])c([2H])c([2H])c2n4-c2cccc(-c3ccccc3)c2-c2nc(-c3ccccc3)nc(-c3ccc4c(c3)oc3ccccc34)n2)c([2H])c1[2H] ; [2H]c1cc([2H])c2oc3c(-c4cc([2H])c(-n5c6cc([2H])c7c8c([2H])c([2H])c([2H])c([2H])c8n(-c8c([2H])c([2H])c([2H])c([2H])c8[2H])c7c6c6c([2H])c([2H])c([2H])c([2H])c65)c(-c5nc(-c6ccccc6)nc(-c6ccccc6)n5)c4[2H])c([2H])c([2H])cc3c2c1]

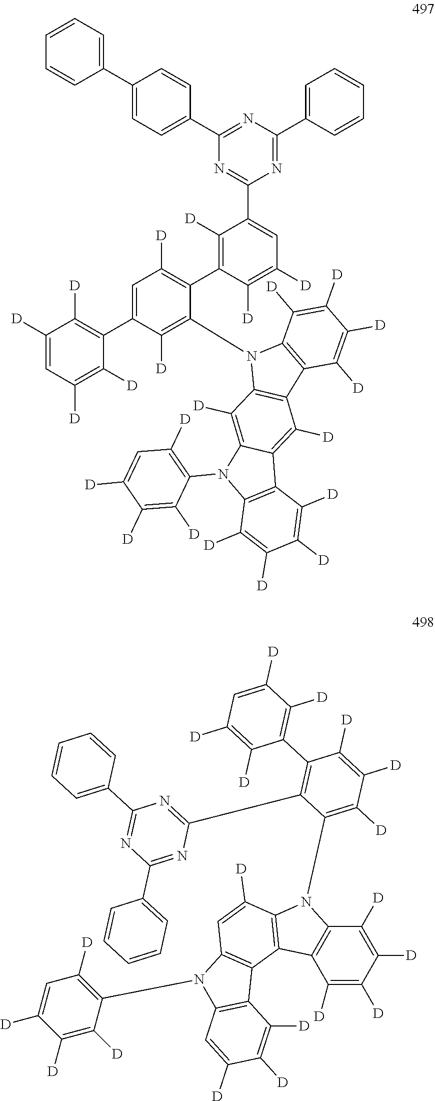 [SMILES: [2H]c1cc([2H])c(-n2c3c([2H])c([2H])c([2H])c([2H])c3c3c([2H])c4c5c([2H])c([2H])c([2H])c([2H])c5n(-c5c([2H])c(-c6c([2H])c([2H])cc([2H])c6[2H])cc([2H])c5-c5c([2H])c([2H])cc(-c6nc(-c7ccccc7)nc(-c7ccc(-c8ccccc8)cc7)n6)c5[2H])c4c([2H])c32)c([2H])c1[2H] ; [2H]c1cc([2H])c(-n2c3cc([2H])c([2H])c([2H])c3c3c4c5c([2H])c([2H])c([2H])c([2H])c5n(-c5c([2H])c([2H])c([2H])c(-c6c([2H])c([2H])cc([2H])c6[2H])c5-c5nc(-c6ccccc6)nc(-c6ccccc6)n5)c4c([2H])cc32)c([2H])c1[2H]]

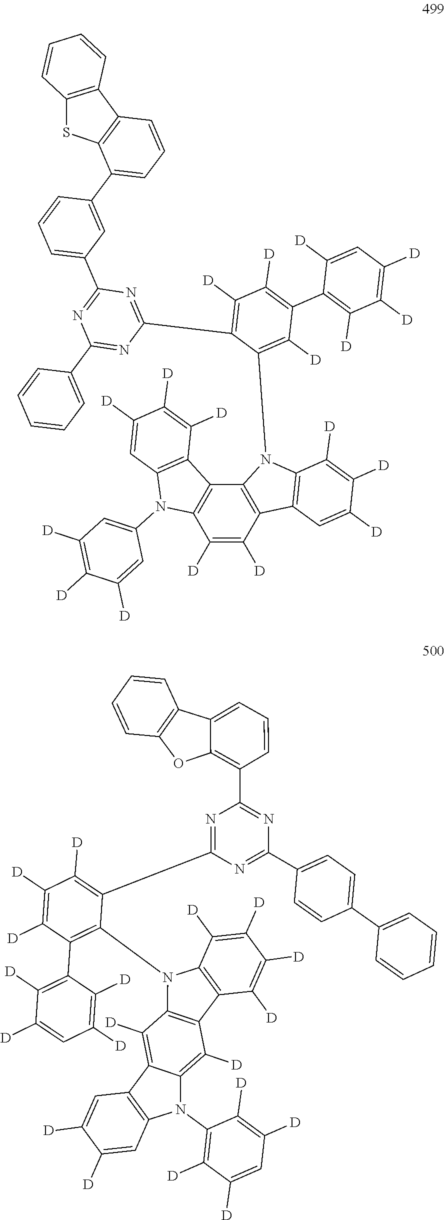 [SMILES: [2H]c1cc(-n2c3cc([2H])c([2H])c([2H])c3c3c2c([2H])c([2H])c2c4cc([2H])c([2H])c([2H])c4n(-c4c([2H])c(-c5c([2H])cc([2H])c([2H])c5[2H])c([2H])c([2H])c4-c4nc(-c5ccccc5)nc(-c5cccc(-c6cccc7c6sc6ccccc67)c5)n4)c23)cc([2H])c1[2H] ; [2H]c1cc2c3c([2H])c4c(c([2H])c3n(-c3c([2H])c([2H])cc([2H])c3[2H])c2cc1[2H])c1c([2H])c([2H])c([2H])c([2H])c1n4-c1c(-c2nc(-c3ccc(-c4ccccc4)cc3)nc(-c3cccc4c3oc3ccccc34)n2)c([2H])c([2H])c([2H])c1-c1c([2H])c([2H])cc([2H])c1[2H]]

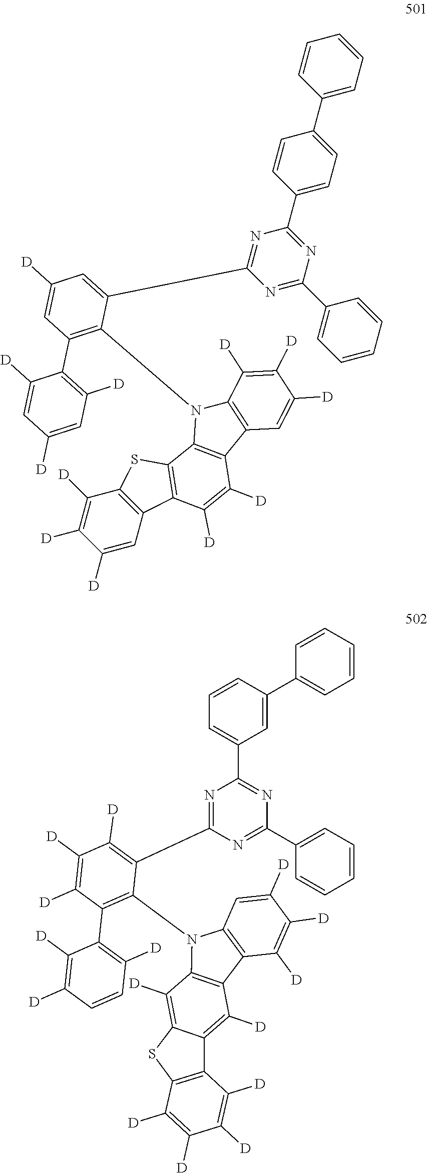 [SMILES: [2H]c1cc([2H])c(-c2cc([2H])cc(-c3nc(-c4ccccc4)nc(-c4ccc(-c5ccccc5)cc4)n3)c2-n2c3c([2H])c([2H])c([2H])cc3c3c([2H])c([2H])c4c5cc([2H])c([2H])c([2H])c5sc4c32)c([2H])c1 ; [2H]c1ccc([2H])c(-c2c([2H])c([2H])c([2H])c(-c3nc(-c4ccccc4)nc(-c4cccc(-c5ccccc5)c4)n3)c2-n2c3cc([2H])c([2H])c([2H])c3c3c([2H])c4c(sc5c([2H])c([2H])c([2H])c([2H])c54)c([2H])c32)c1[2H]]

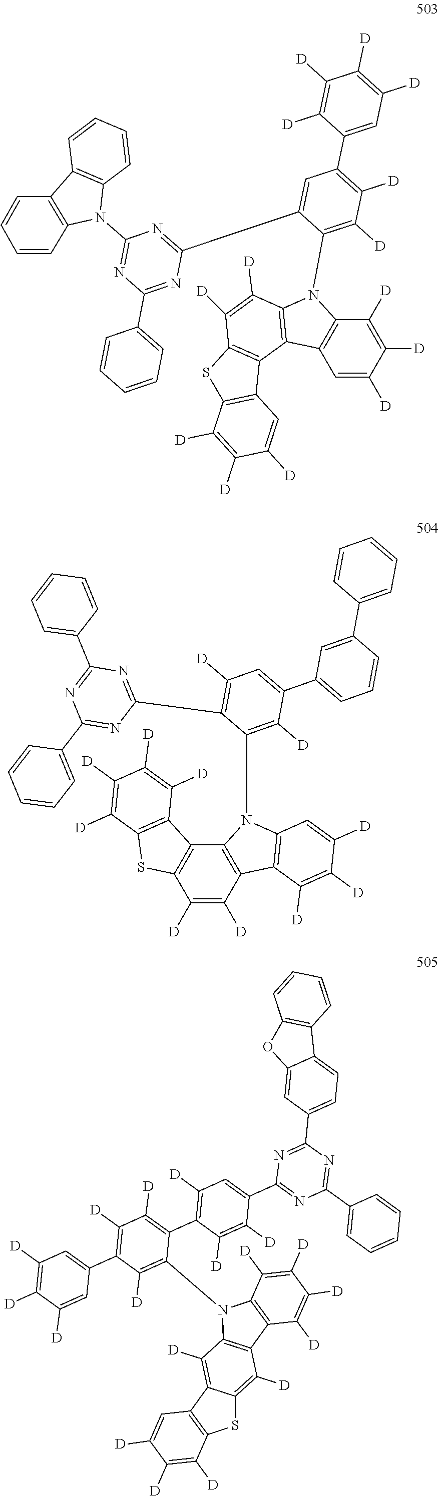

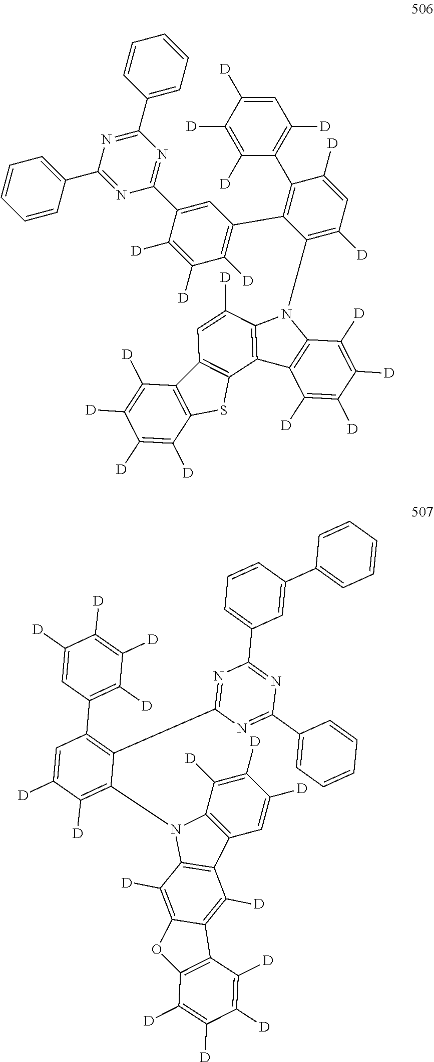 [SMILES: [2H]c1cc([2H])c(-c2c([2H])cc([2H])c(-n3c4c([2H])c([2H])c([2H])c([2H])c4c4c5sc6c([2H])c([2H])c([2H])c([2H])c6c5cc([2H])c43)c2-c2cc(-c3nc(-c4ccccc4)nc(-c4ccccc4)n3)c([2H])c([2H])c2[2H])c([2H])c1[2H] ; [2H]c1cc(-c2cc([2H])c([2H])c(-n3c4c([2H])c([2H])c([2H])cc4c4c([2H])c5c(oc6c([2H])c([2H])c([2H])c([2H])c65)c([2H])c43)c2-c2nc(-c3ccccc3)nc(-c3cccc(-c4ccccc4)c3)n2)c([2H])c([2H])c1[2H]]

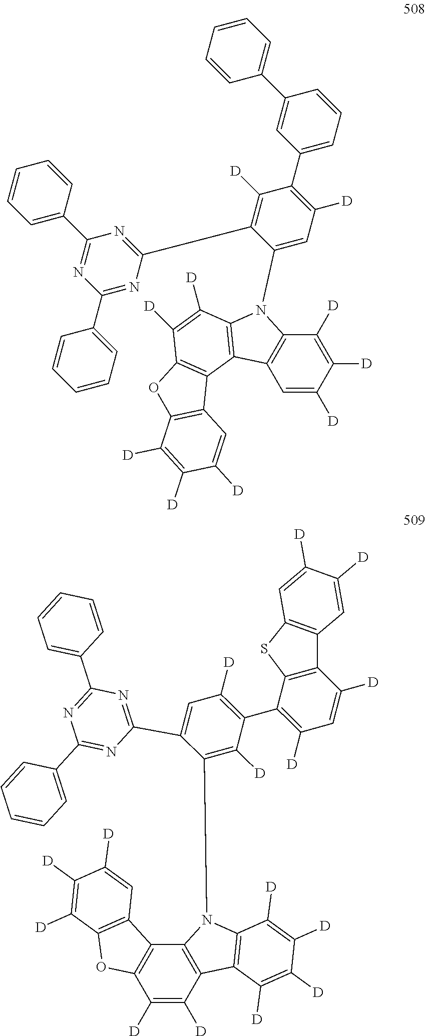 [SMILES: [2H]c1cc2c(oc3c([2H])c([2H])c4c(c5cc([2H])c([2H])c([2H])c5n4-c4cc([2H])c(-c5cccc(-c6ccccc6)c5)c([2H])c4-c4nc(-c5ccccc5)nc(-c5ccccc5)n4)c32)c([2H])c1[2H] ; [2H]c1cc2sc3c(-c4c([2H])cc(-c5nc(-c6ccccc6)nc(-c6ccccc6)n5)c(-n5c6c([2H])c([2H])c([2H])c([2H])c6c6c([2H])c([2H])c7oc8c([2H])c([2H])c([2H])cc8c7c65)c4[2H])c([2H])cc([2H])c3c2cc1[2H]]

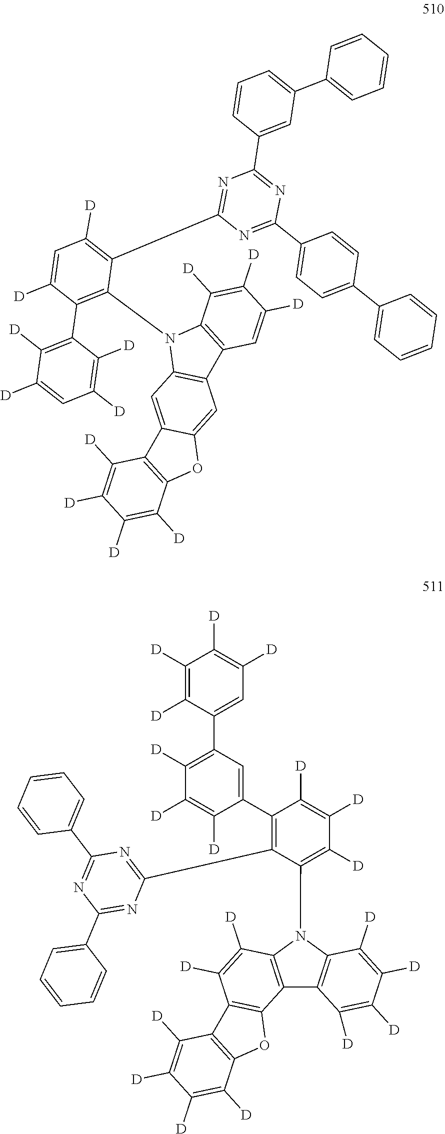 [SMILES: [2H]c1cc([2H])c([2H])c(-c2c([2H])cc([2H])c(-c3nc(-c4ccc(-c5ccccc5)cc4)nc(-c4cccc(-c5ccccc5)c4)n3)c2-n2c3cc4c(cc3c3cc([2H])c([2H])c([2H])c32)oc2c([2H])c([2H])c([2H])c([2H])c24)c1[2H] ; [2H]c1cc(-c2cc(-c3c([2H])c([2H])c([2H])c(-n4c5c([2H])c([2H])c([2H])c([2H])c5c5c6oc7c([2H])c([2H])c([2H])c([2H])c7c6c([2H])c([2H])c54)c3-c3nc(-c4ccccc4)nc(-c4ccccc4)n3)c([2H])c([2H])c2[2H])c([2H])c([2H])c1[2H]]

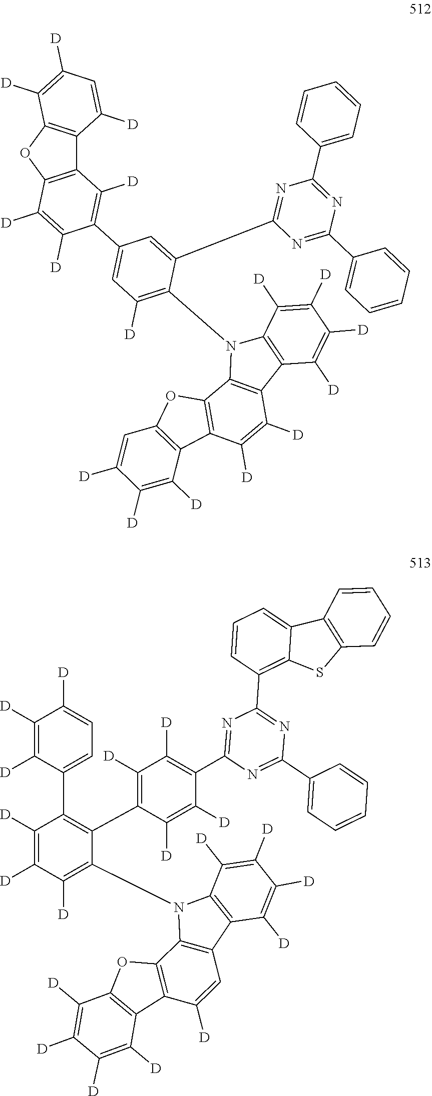 [SMILES: [2H]c1cc2oc3c(c([2H])c([2H])c4c5c([2H])c([2H])c([2H])c([2H])c5n(-c5c([2H])cc(-c6c([2H])c([2H])c7oc8c([2H])c([2H])cc([2H])c8c7c6[2H])cc5-c5nc(-c6ccccc6)nc(-c6ccccc6)n5)c34)c2c([2H])c1[2H] ; [2H]c1ccc(-c2c([2H])c([2H])c([2H])c(-n3c4c([2H])c([2H])c([2H])c([2H])c4c4cc([2H])c5c(oc6c([2H])c([2H])c([2H])c([2H])c65)c43)c2-c2c([2H])c([2H])c(-c3nc(-c4ccccc4)nc(-c4cccc5c4sc4ccccc45)n3)c([2H])c2[2H])c([2H])c1[2H]]

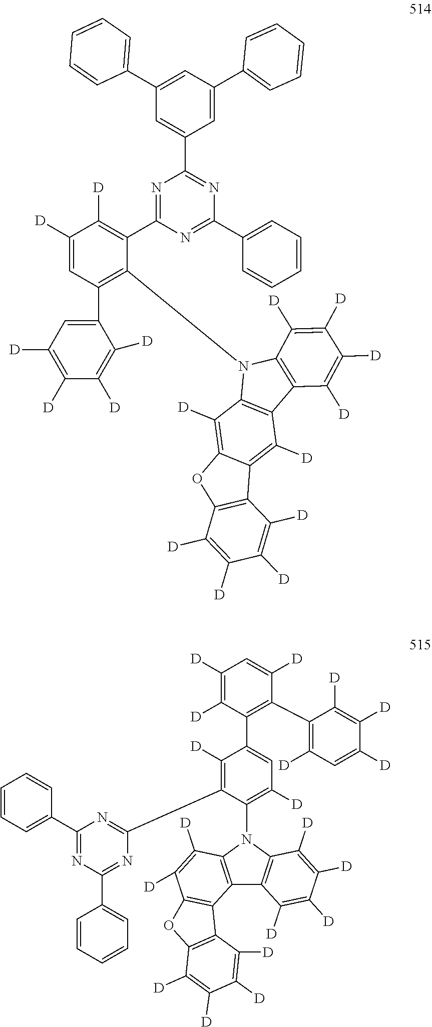 [SMILES: [2H]c1cc(-c2cc([2H])c([2H])c(-c3nc(-c4ccccc4)nc(-c4cc(-c5ccccc5)cc(-c5ccccc5)c4)n3)c2-n2c3c([2H])c([2H])c([2H])c([2H])c3c3c([2H])c4c(oc5c([2H])c([2H])c([2H])c([2H])c54)c([2H])c32)c([2H])c([2H])c1[2H] ; [2H]c1cc([2H])c(-c2c([2H])cc([2H])c([2H])c2-c2cc([2H])c(-n3c4c([2H])c([2H])c([2H])c([2H])c4c4c5c(oc6c([2H])c([2H])c([2H])c([2H])c65)c([2H])c([2H])c43)c(-c3nc(-c4ccccc4)nc(-c4ccccc4)n3)c2[2H])c([2H])c1[2H]]

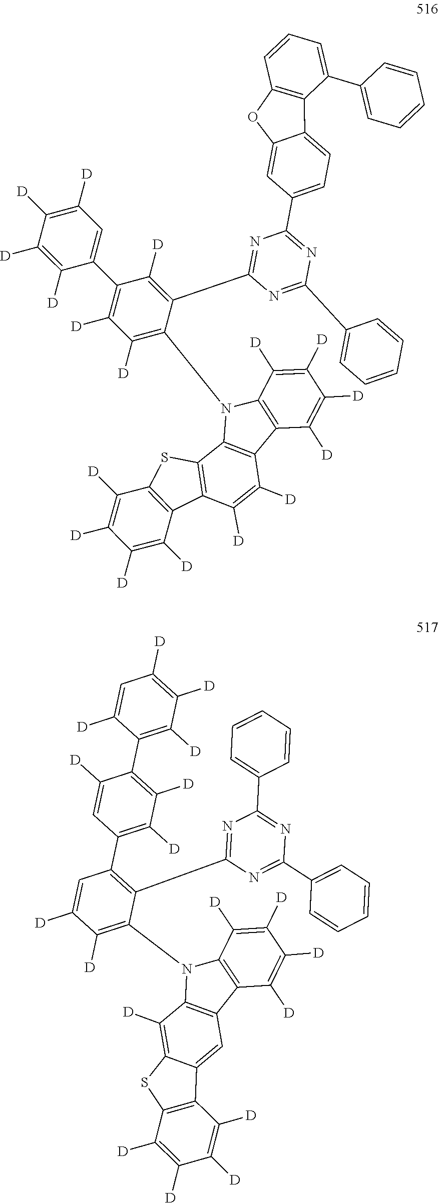 [SMILES: [2H]c1cc(-c2c([2H])c([2H])c(-n3c4c([2H])c([2H])c([2H])c([2H])c4c4c([2H])c([2H])c5c(sc6c([2H])c([2H])c([2H])c([2H])c65)c43)c(-c3nc(-c4ccccc4)nc(-c4ccc5c(c4)oc4cccc(-c6ccccc6)c45)n3)c2[2H])c([2H])c([2H])c1[2H] ; [2H]c1cc([2H])c(-c2c([2H])cc(-c3cc([2H])c([2H])c(-n4c5c([2H])c6sc7c([2H])c([2H])c([2H])c([2H])c7c6cc5c5c([2H])c([2H])c([2H])c([2H])c54)c3-c3nc(-c4ccccc4)nc(-c4ccccc4)n3)c([2H])c2[2H])c([2H])c1[2H]]

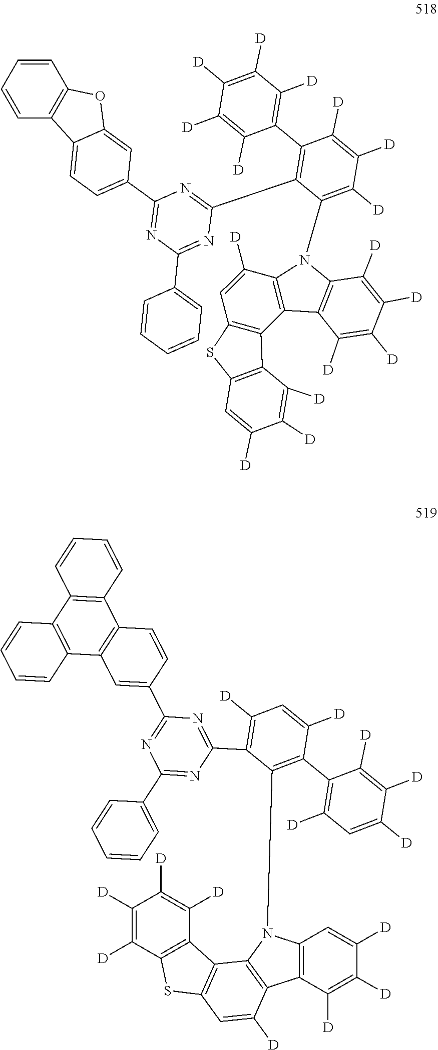 [SMILES: [2H]c1cc2sc3cc([2H])c4c(c5c([2H])c([2H])c([2H])c([2H])c5n4-c4c([2H])c([2H])c([2H])c(-c5c([2H])c([2H])c([2H])c([2H])c5[2H])c4-c4nc(-c5ccccc5)nc(-c5ccc6c(c5)oc5ccccc56)n4)c3c2c([2H])c1[2H] ; [2H]c1cc([2H])c(-c2c([2H])cc([2H])c(-c3nc(-c4ccccc4)nc(-c4ccc5c6ccccc6c6ccccc6c5c4)n3)c2-n2c3cc([2H])c([2H])c([2H])c3c3c([2H])cc4sc5c([2H])c([2H])c([2H])c([2H])c5c4c32)c([2H])c1[2H]]

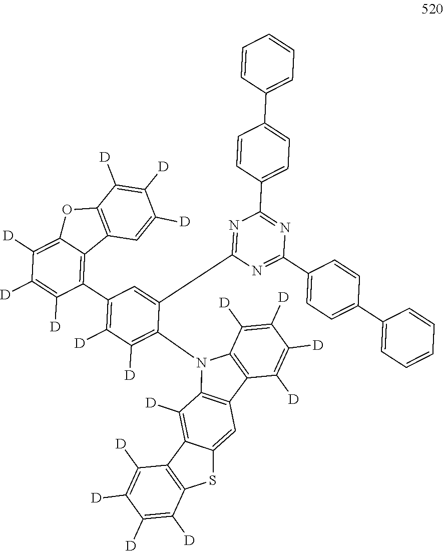 [SMILES: [2H]c1cc2c(oc3c([2H])c([2H])c([2H])c(-c4cc(-c5nc(-c6ccc(-c7ccccc7)cc6)nc(-c6ccc(-c7ccccc7)cc6)n5)c(-n5c6c([2H])c7c(cc6c6c([2H])c([2H])c([2H])c([2H])c65)sc5c([2H])c([2H])c([2H])c([2H])c57)c([2H])c4[2H])c32)c([2H])c1[2H]]

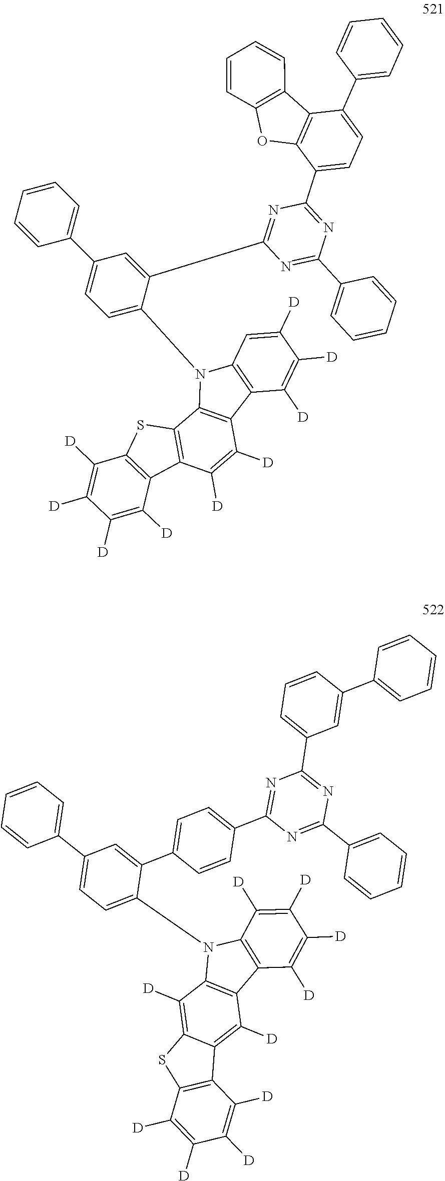 [SMILES: [2H]c1cc2c(c([2H])c1[2H])c1c([2H])c([2H])c3c(sc4c([2H])c([2H])c([2H])c([2H])c43)c1n2-c1ccc(-c2ccccc2)cc1-c1nc(-c2ccccc2)nc(-c2ccc(-c3ccccc3)c3c2oc2ccccc23)n1 ; [2H]c1c([2H])c([2H])c2c(sc3c([2H])c4c(c([2H])c32)c2c([2H])c([2H])c([2H])c([2H])c2n4-c2ccc(-c3ccccc3)cc2-c2ccc(-c3nc(-c4ccccc4)nc(-c4cccc(-c5ccccc5)c4)n3)cc2)c1[2H]]

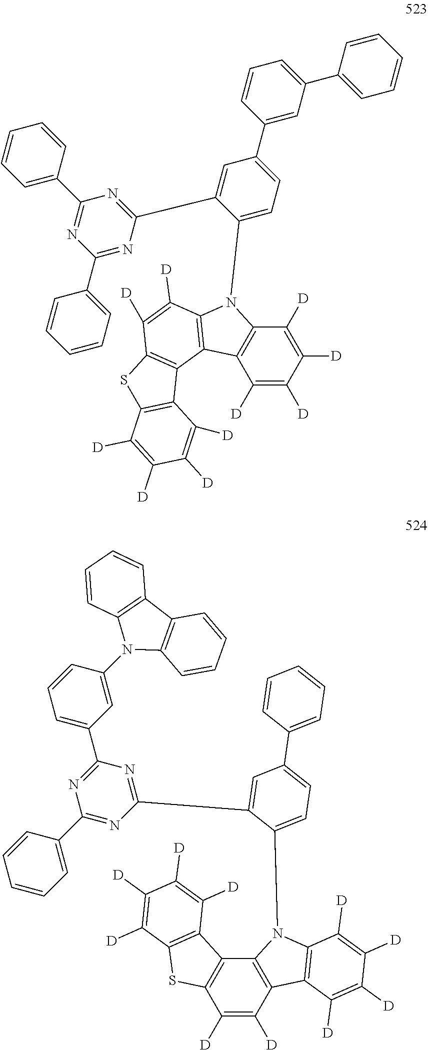 [SMILES: [2H]c1c([2H])c([2H])c2c(sc3c([2H])c([2H])c4c(c5c([2H])c([2H])c([2H])c([2H])c5n4-c4ccc(-c5cccc(-c6ccccc6)c5)cc4-c4nc(-c5ccccc5)nc(-c5ccccc5)n4)c32)c1[2H] ; [2H]c1c([2H])c([2H])c2c(sc3c([2H])c([2H])c4c5c([2H])c([2H])c([2H])c([2H])c5n(-c5ccc(-c6ccccc6)cc5-c5nc(-c6ccccc6)nc(-c6cccc(-n7c8ccccc8c8ccccc87)c6)n5)c4c32)c1[2H]]

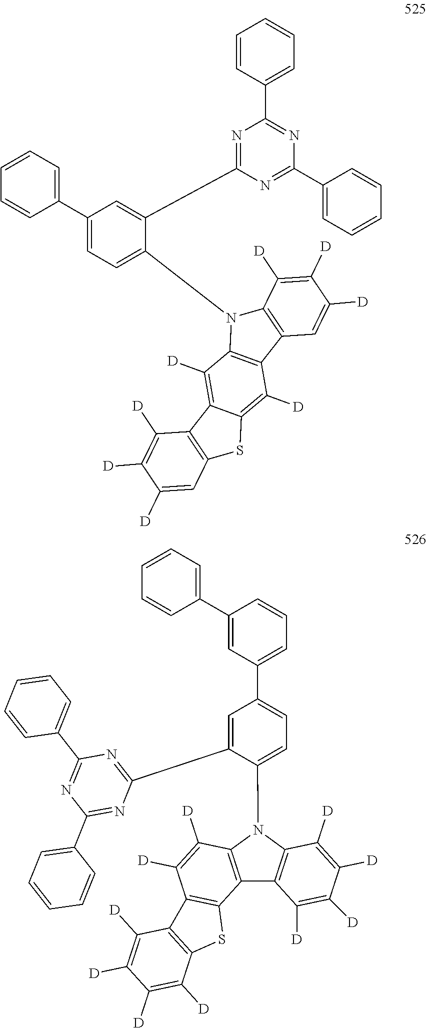

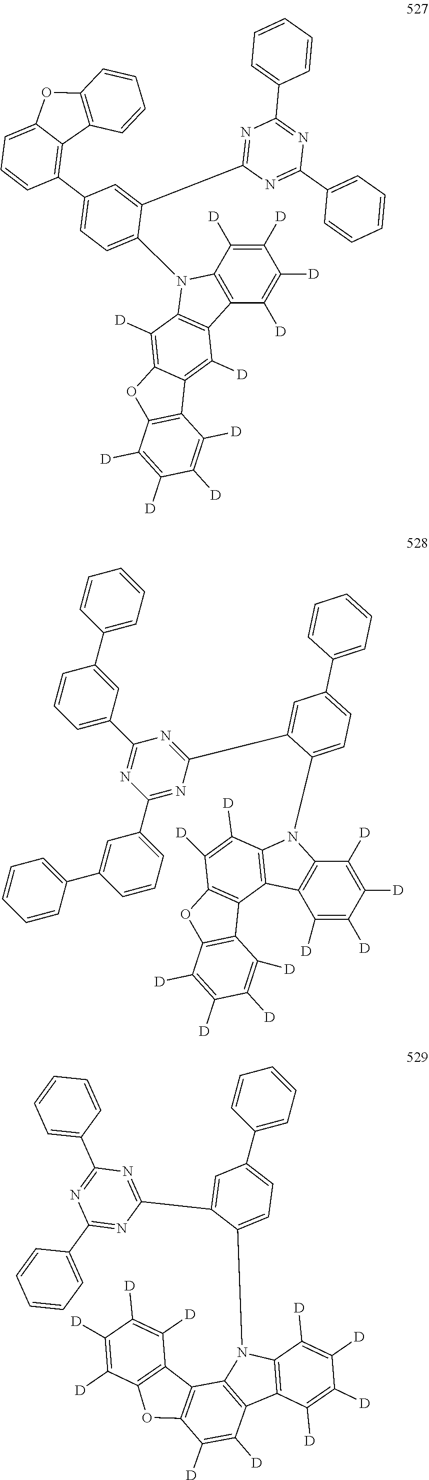 [SMILES: [2H]c1c([2H])c([2H])c2c(oc3c([2H])c4c(c([2H])c32)c2c([2H])c([2H])c([2H])c([2H])c2n4-c2ccc(-c3cccc4oc5ccccc5c34)cc2-c2nc(-c3ccccc3)nc(-c3ccccc3)n2)c1[2H] ; [2H]c1c([2H])c([2H])c2c(oc3c([2H])c([2H])c4c(c5c([2H])c([2H])c([2H])c([2H])c5n4-c4ccc(-c5ccccc5)cc4-c4nc(-c5cccc(-c6ccccc6)c5)nc(-c5cccc(-c6ccccc6)c5)n4)c32)c1[2H] ; [2H]c1c([2H])c([2H])c2c(oc3c([2H])c([2H])c4c5c([2H])c([2H])c([2H])c([2H])c5n(-c5ccc(-c6ccccc6)cc5-c5nc(-c6ccccc6)nc(-c6ccccc6)n5)c4c32)c1[2H]]

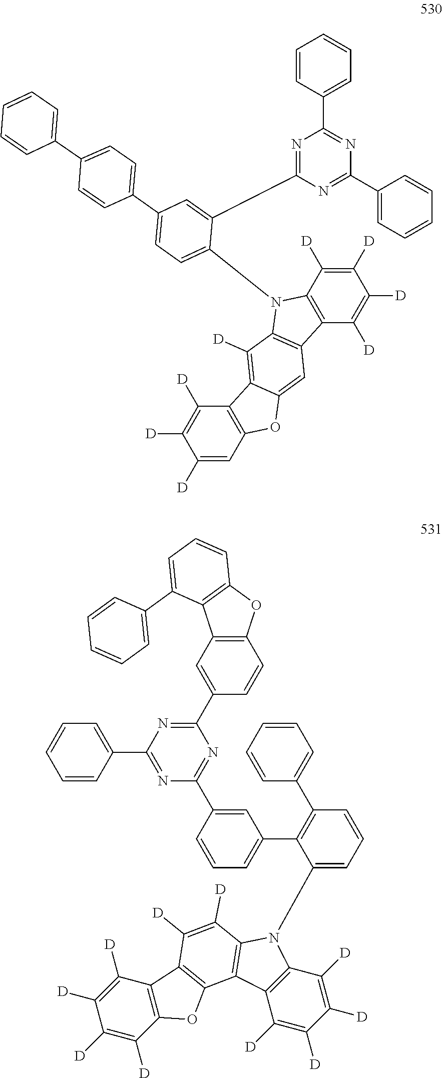 [SMILES: [2H]c1cc2oc3cc4c5c([2H])c([2H])c([2H])c([2H])c5n(-c5ccc(-c6ccc(-c7ccccc7)cc6)cc5-c5nc(-c6ccccc6)nc(-c6ccccc6)n5)c4c([2H])c3c2c([2H])c1[2H] ; [2H]c1c([2H])c([2H])c2c(oc3c2c([2H])c([2H])c2c3c3c([2H])c([2H])c([2H])c([2H])c3n2-c2cccc(-c3ccccc3)c2-c2cccc(-c3nc(-c4ccccc4)nc(-c4ccc5oc6cccc(-c7ccccc7)c6c5c4)n3)c2)c1[2H]]

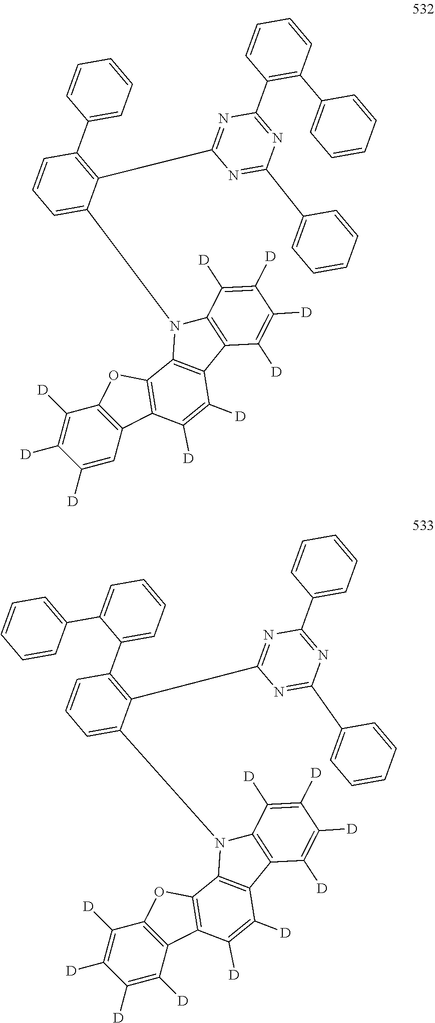 [SMILES: [2H]c1cc2c(oc3c2c([2H])c([2H])c2c4c([2H])c([2H])c([2H])c([2H])c4n(-c4cccc(-c5ccccc5)c4-c4nc(-c5ccccc5)nc(-c5ccccc5-c5ccccc5)n4)c32)c([2H])c1[2H] ; [2H]c1c([2H])c([2H])c2c(oc3c2c([2H])c([2H])c2c4c([2H])c([2H])c([2H])c([2H])c4n(-c4cccc(-c5ccccc5-c5ccccc5)c4-c4nc(-c5ccccc5)nc(-c5ccccc5)n4)c32)c1[2H]]

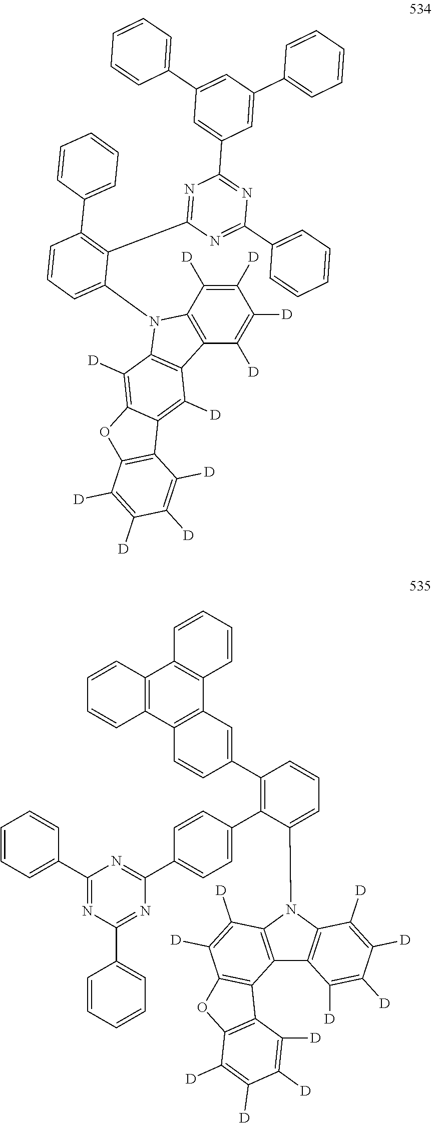 [SMILES: [2H]c1c([2H])c([2H])c2c(oc3c([2H])c4c(c([2H])c32)c2c([2H])c([2H])c([2H])c([2H])c2n4-c2cccc(-c3ccccc3)c2-c2nc(-c3ccccc3)nc(-c3cc(-c4ccccc4)cc(-c4ccccc4)c3)n2)c1[2H] ; [2H]c1c([2H])c([2H])c2c(oc3c([2H])c([2H])c4c(c5c([2H])c([2H])c([2H])c([2H])c5n4-c4cccc(-c5ccc6c7ccccc7c7ccccc7c6c5)c4-c4ccc(-c5nc(-c6ccccc6)nc(-c6ccccc6)n5)cc4)c32)c1[2H]]

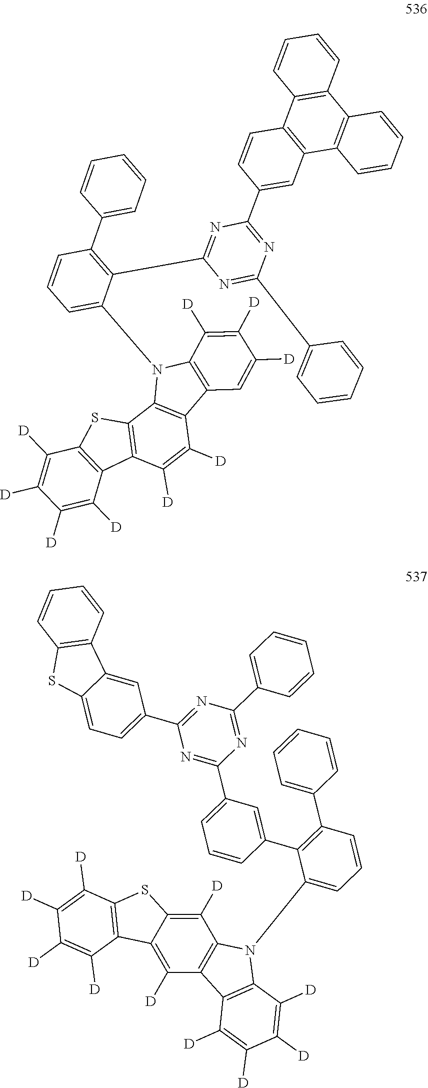 [SMILES: [2H]c1cc2c3c([2H])c([2H])c4c(sc5c([2H])c([2H])c([2H])c([2H])c54)c3n(-c3cccc(-c4ccccc4)c3-c3nc(-c4ccccc4)nc(-c4ccc5c6ccccc6c6ccccc6c5c4)n3)c2c([2H])c1[2H] ; [2H]c1c([2H])c([2H])c2c(sc3c([2H])c4c(c([2H])c32)c2c([2H])c([2H])c([2H])c([2H])c2n4-c2cccc(-c3ccccc3)c2-c2cccc(-c3nc(-c4ccccc4)nc(-c4ccc5sc6ccccc6c5c4)n3)c2)c1[2H]]

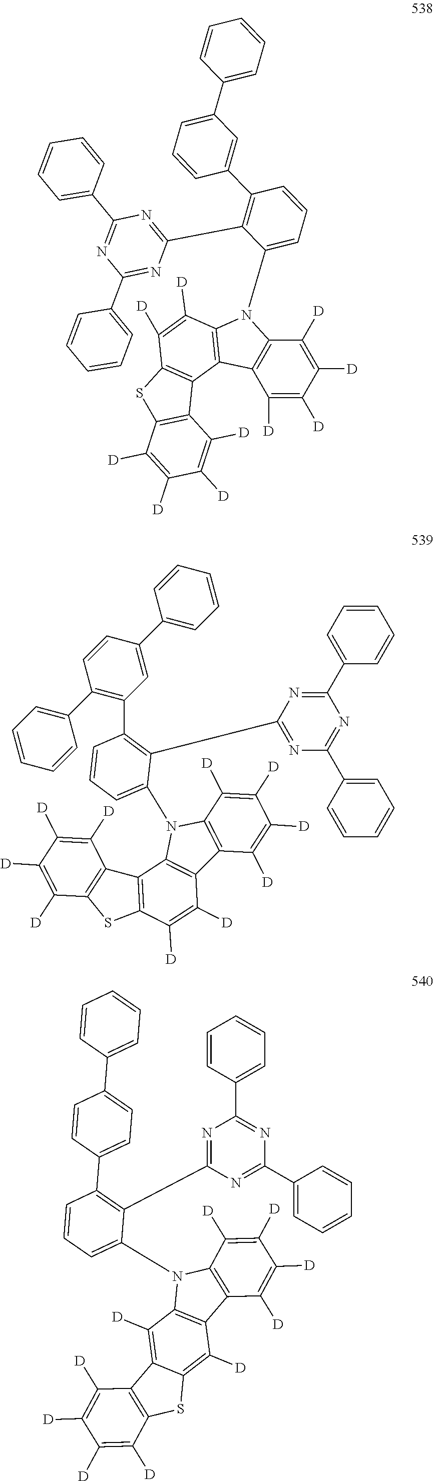 [SMILES: [2H]c1c([2H])c([2H])c2c(sc3c([2H])c([2H])c4c(c5c([2H])c([2H])c([2H])c([2H])c5n4-c4cccc(-c5cccc(-c6ccccc6)c5)c4-c4nc(-c5ccccc5)nc(-c5ccccc5)n4)c32)c1[2H] ; [2H]c1c([2H])c([2H])c2c(sc3c([2H])c([2H])c4c5c([2H])c([2H])c([2H])c([2H])c5n(-c5cccc(-c6cc(-c7ccccc7)ccc6-c6ccccc6)c5-c5nc(-c6ccccc6)nc(-c6ccccc6)n5)c4c32)c1[2H] ; [2H]c1c([2H])c([2H])c2c(sc3c([2H])c4c5c([2H])c([2H])c([2H])c([2H])c5n(-c5cccc(-c6ccc(-c7ccccc7)cc6)c5-c5nc(-c6ccccc6)nc(-c6ccccc6)n5)c4c([2H])c32)c1[2H]]

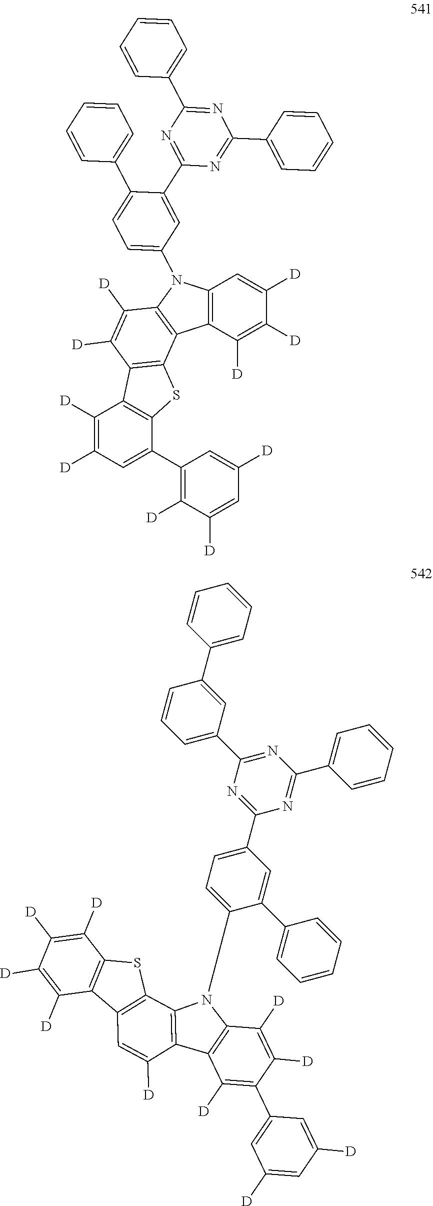

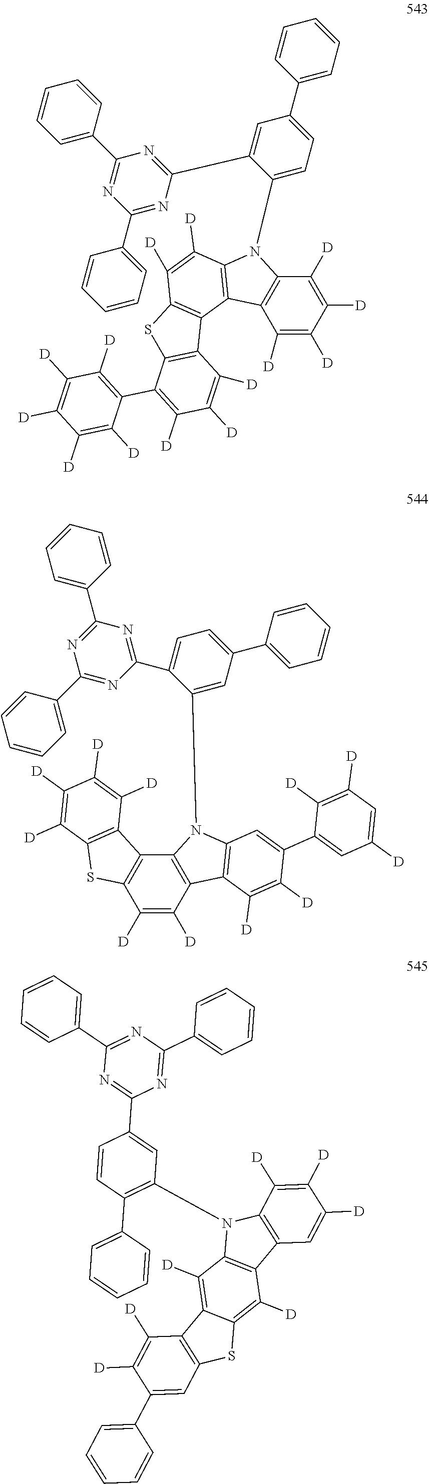 [SMILES: [2H]c1c([2H])c([2H])c(-c2c([2H])c([2H])c([2H])c3c2sc2c([2H])c([2H])c4c(c5c([2H])c([2H])c([2H])c([2H])c5n4-c4ccc(-c5ccccc5)cc4-c4nc(-c5ccccc5)nc(-c5ccccc5)n4)c23)c([2H])c1[2H] ; [2H]c1cc([2H])c([2H])c(-c2cc3c(c([2H])c2[2H])c2c([2H])c([2H])c4sc5c([2H])c([2H])c([2H])c([2H])c5c4c2n3-c2cc(-c3ccccc3)ccc2-c2nc(-c3ccccc3)nc(-c3ccccc3)n2)c1 ; [2H]c1cc2c3c([2H])c4sc5cc(-c6ccccc6)c([2H])c([2H])c5c4c([2H])c3n(-c3cc(-c4nc(-c5ccccc5)nc(-c5ccccc5)n4)ccc3-c3ccccc3)c2c([2H])c1[2H]]

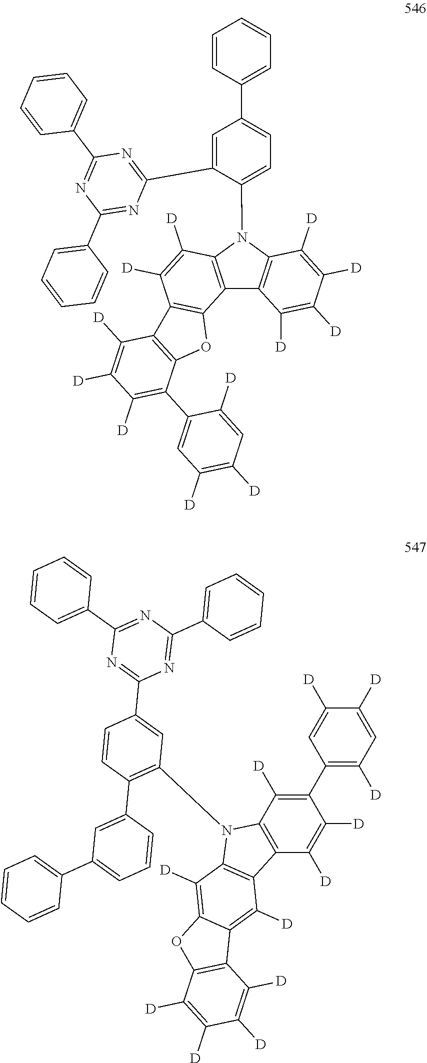 [SMILES: [2H]c1cc([2H])c(-c2c([2H])c([2H])c([2H])c3c2oc2c3c([2H])c([2H])c3c2c2c([2H])c([2H])c([2H])c([2H])c2n3-c2ccc(-c3ccccc3)cc2-c2nc(-c3ccccc3)nc(-c3ccccc3)n2)cc1[2H] ; [2H]c1cc([2H])c(-c2c([2H])c([2H])c3c4c([2H])c5c(oc6c([2H])c([2H])c([2H])c([2H])c65)c([2H])c4n(-c4cc(-c5nc(-c6ccccc6)nc(-c6ccccc6)n5)ccc4-c4cccc(-c5ccccc5)c4)c3c2[2H])cc1[2H]]

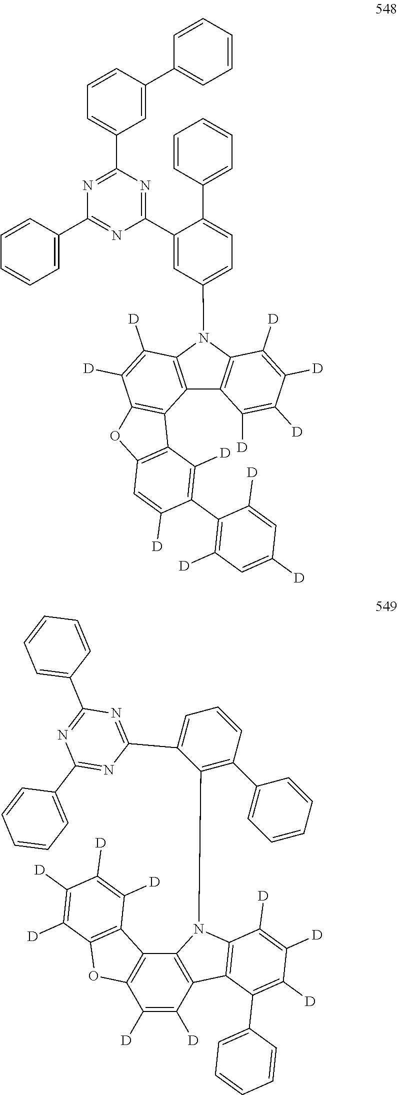 [SMILES: [2H]c1cc([2H])c(-c2c([2H])cc3oc4c([2H])c([2H])c5c(c6c([2H])c([2H])c([2H])c([2H])c6n5-c5ccc(-c6ccccc6)c(-c6nc(-c7ccccc7)nc(-c7cccc(-c8ccccc8)c7)n6)c5)c4c3c2[2H])c([2H])c1 ; [2H]c1c([2H])c([2H])c2c(oc3c([2H])c([2H])c4c5c(-c6ccccc6)c([2H])c([2H])c([2H])c5n(-c5c(-c6ccccc6)cccc5-c5nc(-c6ccccc6)nc(-c6ccccc6)n5)c4c32)c1[2H]]

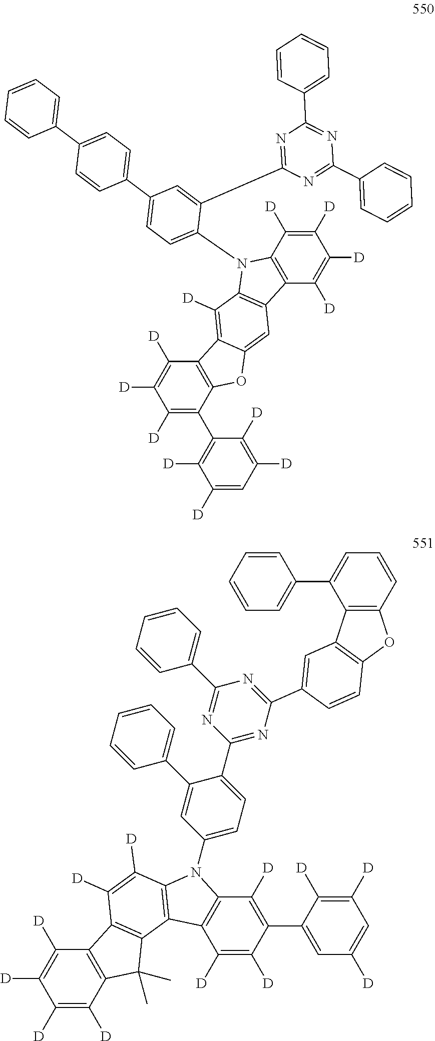 [SMILES: [2H]c1cc([2H])c([2H])c(-c2c([2H])c([2H])c([2H])c3c2oc2cc4c5c([2H])c([2H])c([2H])c([2H])c5n(-c5ccc(-c6ccc(-c7ccccc7)cc6)cc5-c5nc(-c6ccccc6)nc(-c6ccccc6)n5)c4c([2H])c23)c1[2H] ; [2H]c1cc([2H])c([2H])c(-c2c([2H])c([2H])c3c4c5c(c([2H])c([2H])c4n(-c4ccc(-c6nc(-c7ccccc7)nc(-c7ccc8oc9cccc(-c%10ccccc%10)c9c8c7)n6)c(-c6ccccc6)c4)c3c2[2H])-c2c([2H])c([2H])c([2H])c([2H])c2C5(C)C)c1]

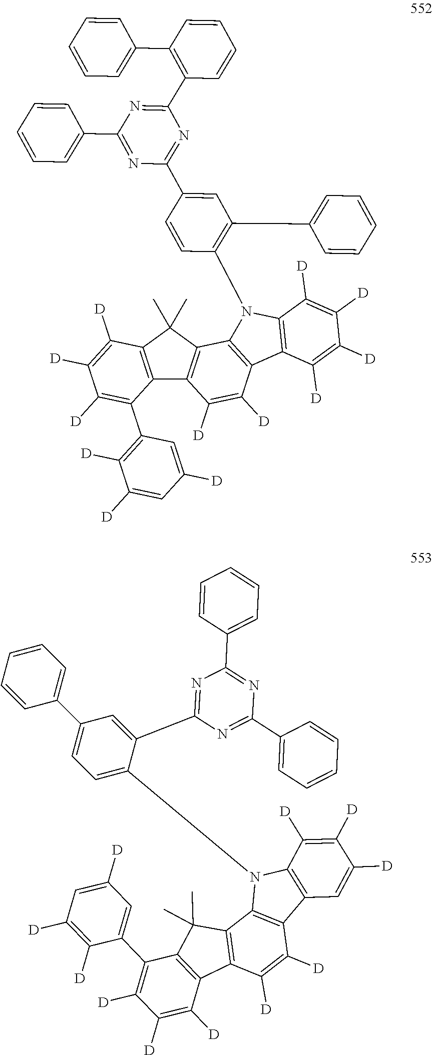 [SMILES: [2H]c1cc([2H])c([2H])c(-c2c([2H])c([2H])c([2H])c3c2-c2c([2H])c([2H])c4c5c([2H])c([2H])c([2H])c([2H])c5n(-c5ccc(-c6nc(-c7ccccc7)nc(-c7ccccc7-c7ccccc7)n6)cc5-c5ccccc5)c4c2C3(C)C)c1 ; [2H]c1cc([2H])c([2H])c(-c2c([2H])c([2H])c([2H])c3c2C(C)(C)c2c-3c([2H])c([2H])c3c4cc([2H])c([2H])c([2H])c4n(-c4ccc(-c5ccccc5)cc4-c4nc(-c5ccccc5)nc(-c5ccccc5)n4)c23)c1]

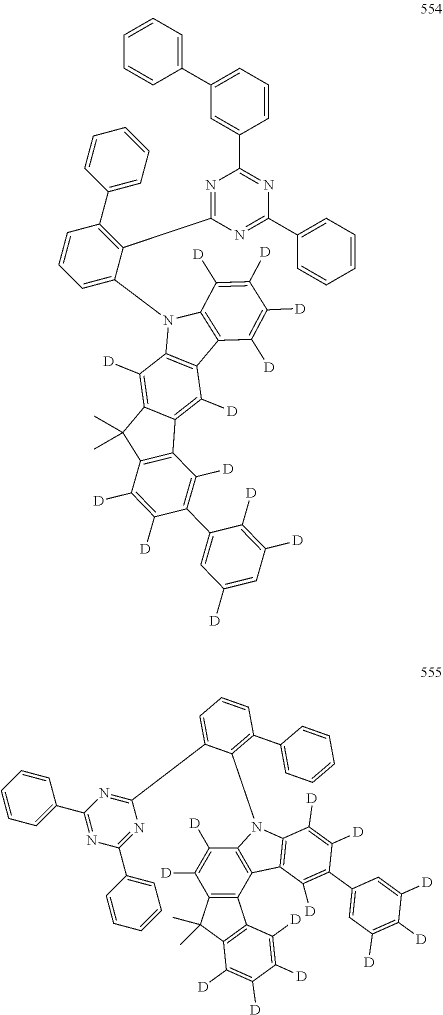 [SMILES: [2H]c1cc([2H])c([2H])c(-c2c([2H])c([2H])c3c(c2[2H])-c2c(c([2H])c4c(c2[2H])c2c([2H])c([2H])c([2H])c([2H])c2n4-c2cccc(-c4ccccc4)c2-c2nc(-c4ccccc4)nc(-c4cccc(-c5ccccc5)c4)n2)C3(C)C)c1 ; [2H]c1cc(-c2c([2H])c([2H])c3c(c2[2H])c2c4c(c([2H])c([2H])c2n3-c2c(-c3ccccc3)cccc2-c2nc(-c3ccccc3)nc(-c3ccccc3)n2)C(C)(C)c2c([2H])c([2H])c([2H])c([2H])c2-4)cc([2H])c1[2H]]

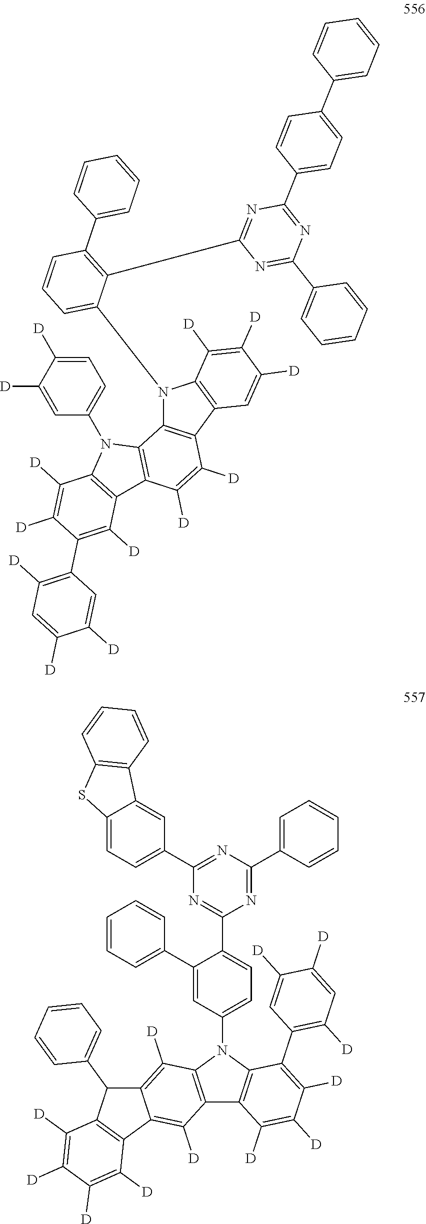 [SMILES: [2H]c1ccc(-n2c3c([2H])c([2H])c(-c4cc([2H])c([2H])cc4[2H])c([2H])c3c3c([2H])c([2H])c4c5cc([2H])c([2H])c([2H])c5n(-c5cccc(-c6ccccc6)c5-c5nc(-c6ccccc6)nc(-c6ccc(-c7ccccc7)cc6)n5)c4c32)cc1[2H] ; [2H]c1cc([2H])c(-c2c([2H])c([2H])c([2H])c3c4c([2H])c5c(c([2H])c4n(-c4ccc(-c6nc(-c7ccccc7)nc(-c7ccc8sc9ccccc9c8c7)n6)c(-c6ccccc6)c4)c23)C(c2ccccc2)c2c([2H])c([2H])c([2H])c([2H])c2-5)cc1[2H]]

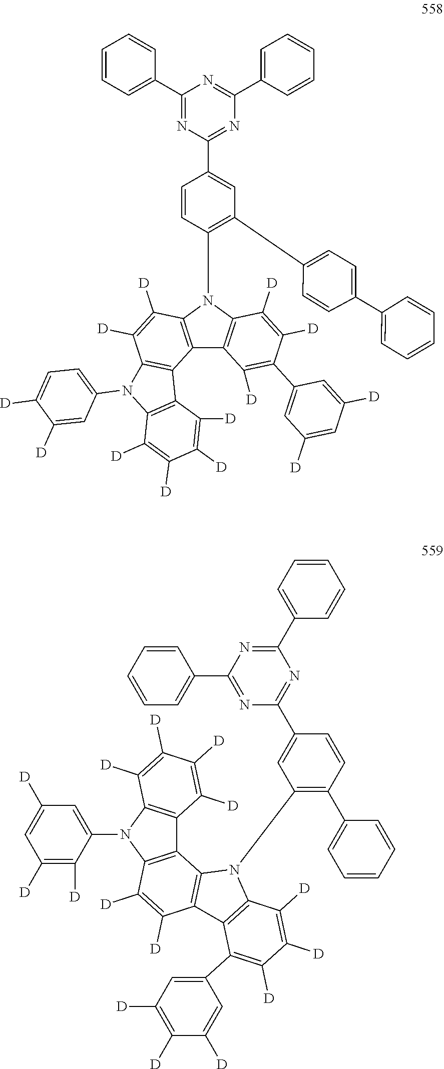 [SMILES: [2H]c1cc([2H])cc(-c2c([2H])c([2H])c3c(c2[2H])c2c4c5c([2H])c([2H])c([2H])c([2H])c5n(-c5ccc([2H])c([2H])c5)c4c([2H])c([2H])c2n3-c2ccc(-c3nc(-c4ccccc4)nc(-c4ccccc4)n3)cc2-c2ccc(-c3ccccc3)cc2)c1 ; [2H]c1cc([2H])c([2H])c(-n2c3c([2H])c([2H])c([2H])c([2H])c3c3c2c([2H])c([2H])c2c4c(-c5cc([2H])c([2H])c([2H])c5)c([2H])c([2H])c([2H])c4n(-c4cc(-c5nc(-c6ccccc6)nc(-c6ccccc6)n5)ccc4-c4ccccc4)c23)c1]

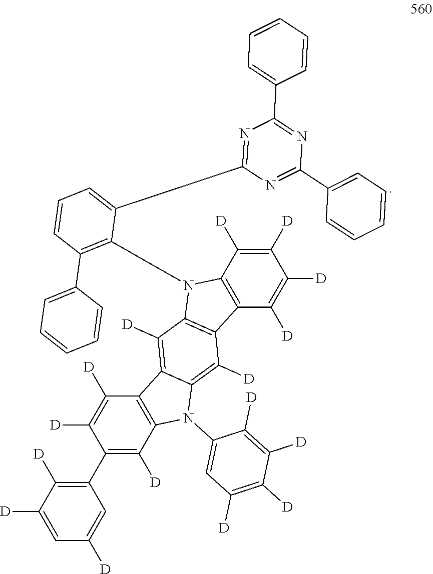 [SMILES: [2H]c1cc([2H])c([2H])c(-c2c([2H])c([2H])c3c4c([2H])c5c(c([2H])c4n(-c4cc([2H])c([2H])c([2H])c4[2H])c3c2[2H])c2c([2H])c([2H])c([2H])c([2H])c2n5-c2c(-c3ccccc3)cccc2-c2nc(-c3ccccc3)nc(-c3ccccc3)n2)c1]

7. An organic light emitting device comprising: a first electrode; a second electrode; and an organic material layer having one or more layers provided between the first electrode and the second electrode,
wherein one or more layers of the organic material layer comprise one or more of the heterocyclic compounds of claim 1.

8. The organic light emitting device of claim 7, wherein the organic material layer comprises a light emitting layer, and the light emitting layer comprises one or more of the heterocyclic compounds.

9. The organic light emitting device of claim 8, wherein the light emitting layer comprises a host, and the host comprises one or more of the heterocyclic compounds.

10. The organic light emitting device of claim 8, wherein the light emitting layer further comprises a compound of the following Chemical Formula 2 or 3: 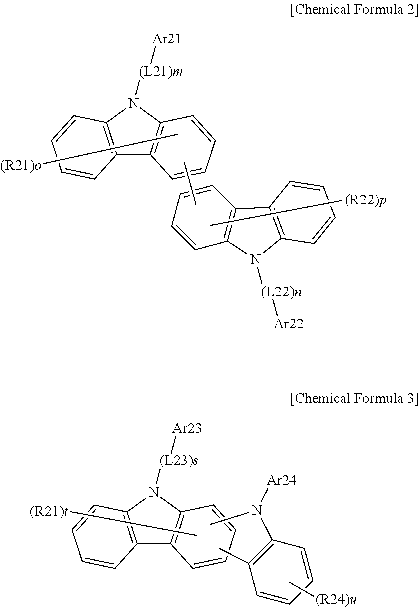
in Chemical Formulae 2 and 3,
L21 to L23 are each independently a direct bond; a substituted or unsubstituted C6 to C60 arylene group; or a substituted or unsubstituted C2 to C60 heteroarylene group,
m, n, and s are each an integer from 1 to 3, and when m, n, and s are each 2 or greater, substituents in the parenthesis are the same as or different from each other,
Ar21 to Ar24 are each independently a substituted or unsubstituted C6 to C60 aryl group; or a substituted or unsubstituted C2 to C60 heteroaryl group,
R21 to R24 are each independently hydrogen; deuterium; a halogen group; a cyano group; a substituted or unsubstituted C1 to C60 alkyl group; a substituted or unsubstituted C6 to C60 aryl group; or a substituted or unsubstituted C2 to C60 heteroaryl group, and
o and p are each an integer from 1 to 7, t is an integer from 1 to 6, u is an integer from 1 to 4, and when o, p, t, and u are each 2 or greater, substituents in the parenthesis are the same as or different from each other.

11. The organic light emitting device of claim 10, wherein Chemical Formula 2 is represented by any one of the following compounds:

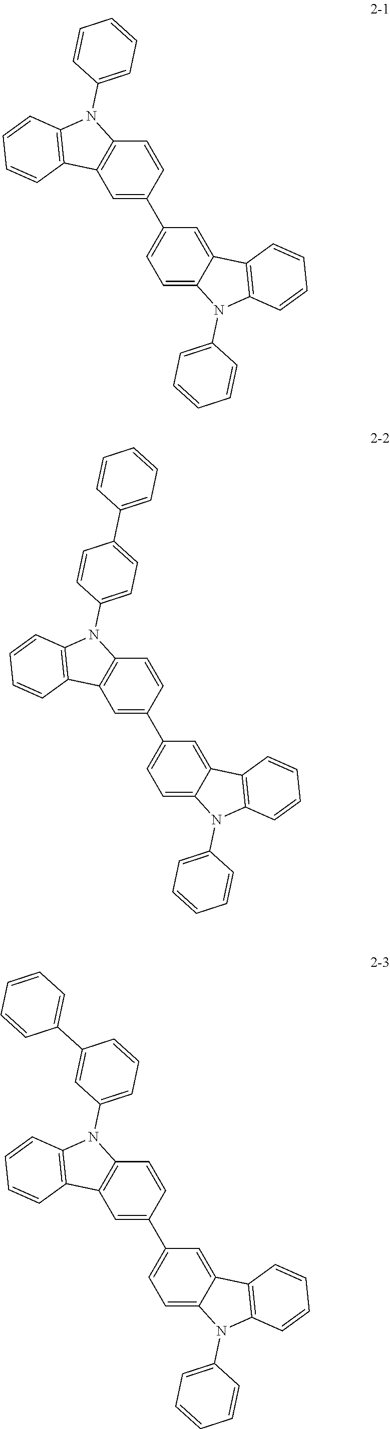 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1]

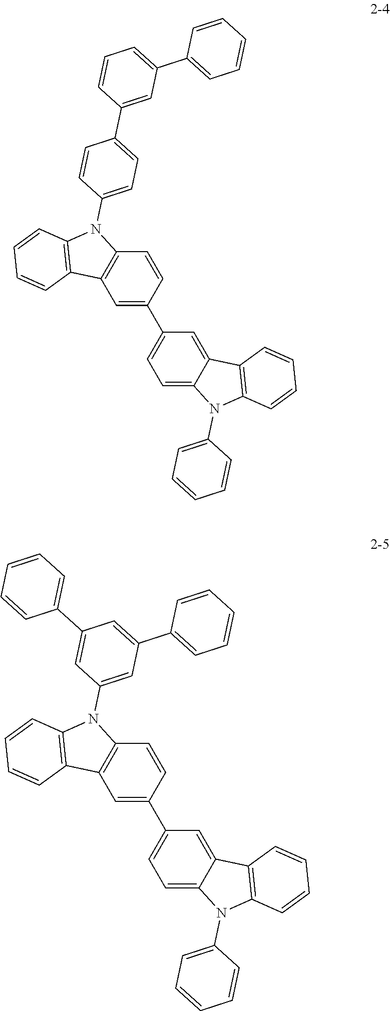 [SMILES: c1ccc(-c2cccc(-c3ccc(-n4c5ccccc5c5cc(-c6ccc7c(c6)c6ccccc6n7-c6ccccc6)ccc54)cc3)c2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

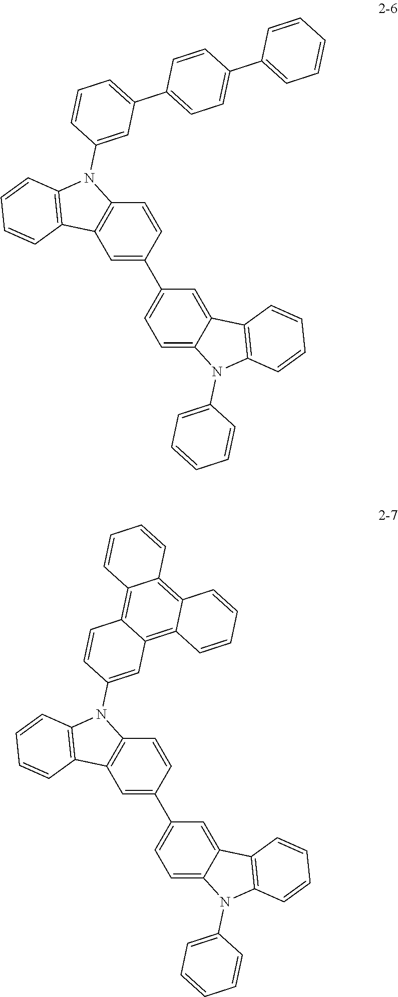 [SMILES: c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-n2c3ccccc3c3cc(-c4ccc5c(c4)c4ccccc4n5-c4ccc5c6ccccc6c6ccccc6c5c4)ccc32)cc1]

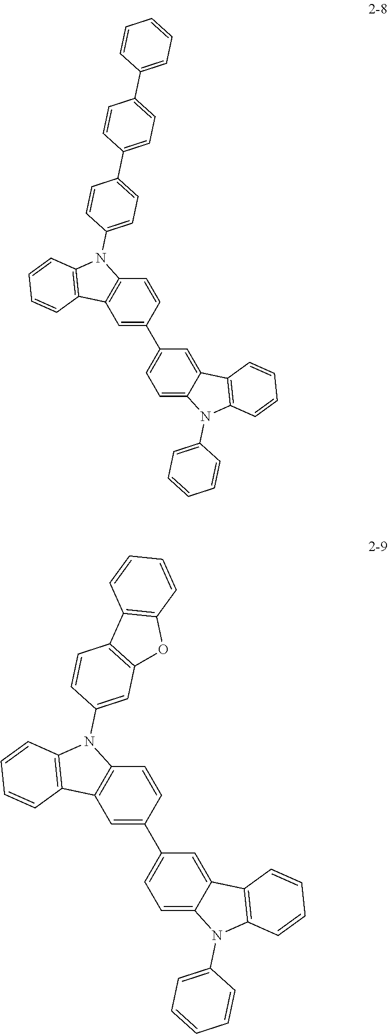 [SMILES: c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-n2c3ccccc3c3cc(-c4ccc5c(c4)c4ccccc4n5-c4ccc5c(c4)oc4ccccc45)ccc32)cc1]

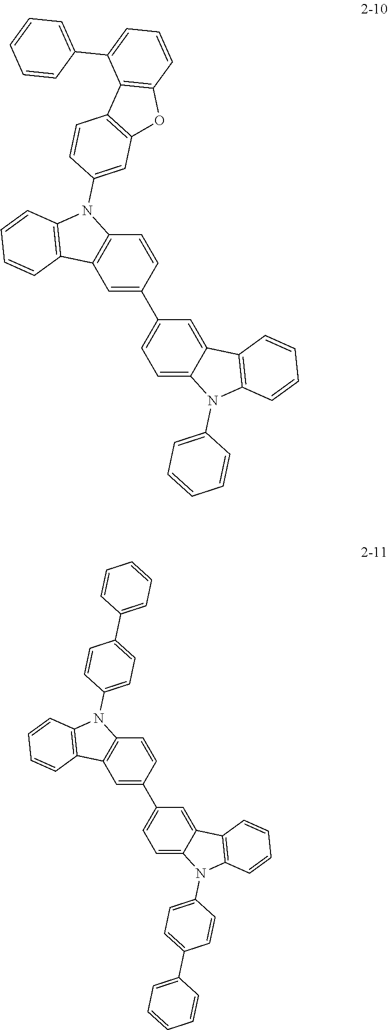 [SMILES: c1ccc(-c2cccc3oc4cc(-n5c6ccccc6c6cc(-c7ccc8c(c7)c7ccccc7n8-c7ccccc7)ccc65)ccc4c23)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

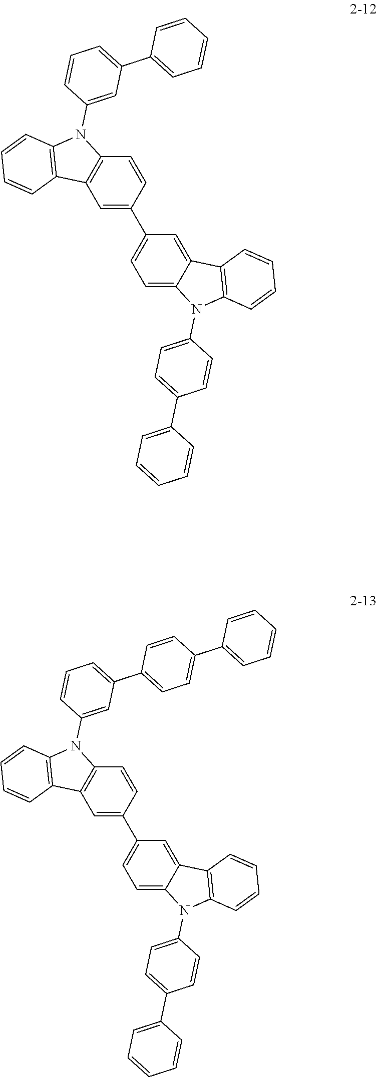 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1]

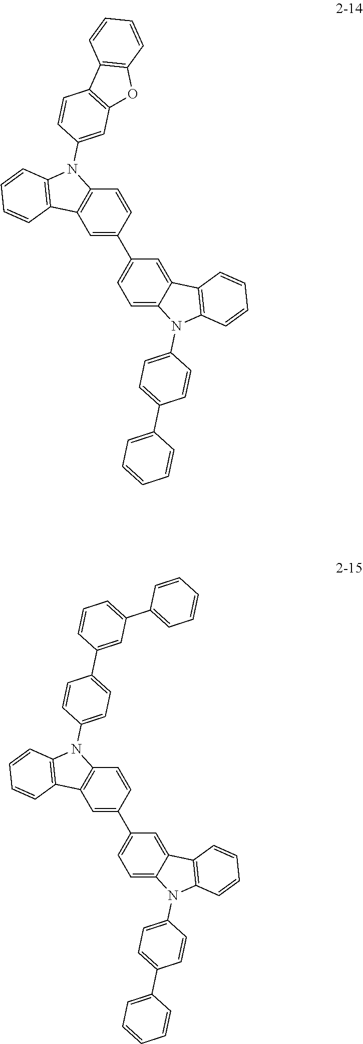 [SMILES: c1ccc(-c2ccc(-n3c4ccccc4c4cc(-c5ccc6c(c5)c5ccccc5n6-c5ccc6c(c5)oc5ccccc56)ccc43)cc2)cc1 ; c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1]

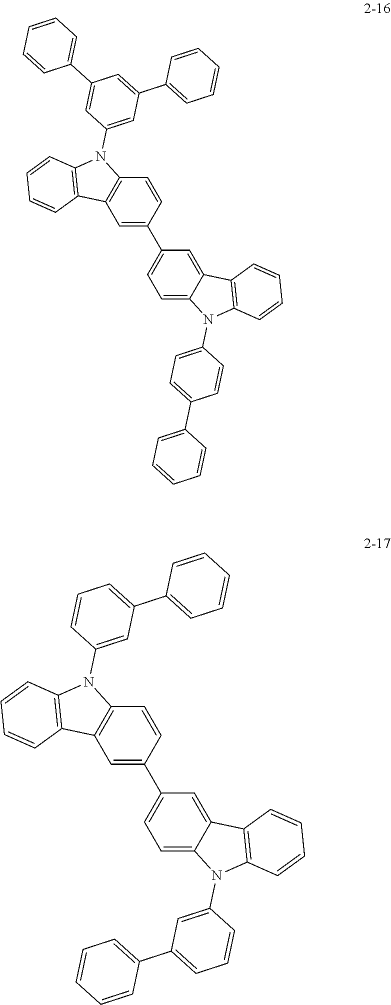 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

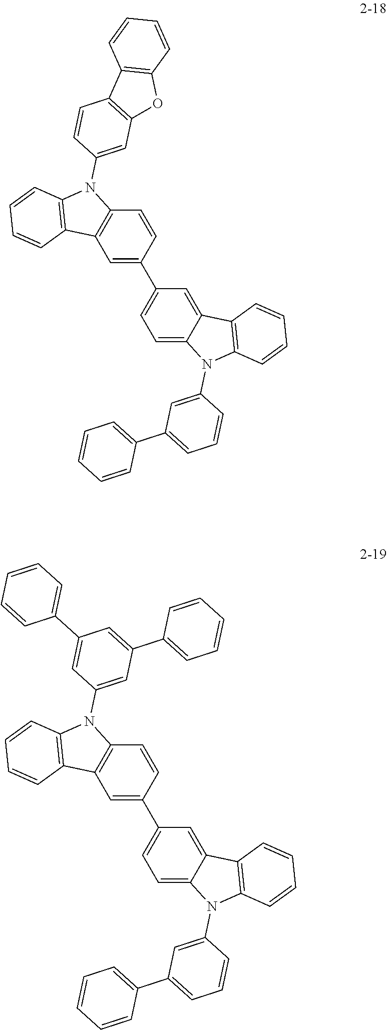 [SMILES: c1ccc(-c2cccc(-n3c4ccccc4c4cc(-c5ccc6c(c5)c5ccccc5n6-c5ccc6c(c5)oc5ccccc56)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

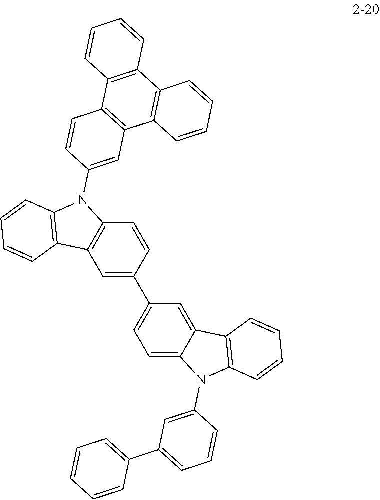 [SMILES: c1ccc(-c2cccc(-n3c4ccccc4c4cc(-c5ccc6c(c5)c5ccccc5n6-c5ccc6c7ccccc7c7ccccc7c6c5)ccc43)c2)cc1]

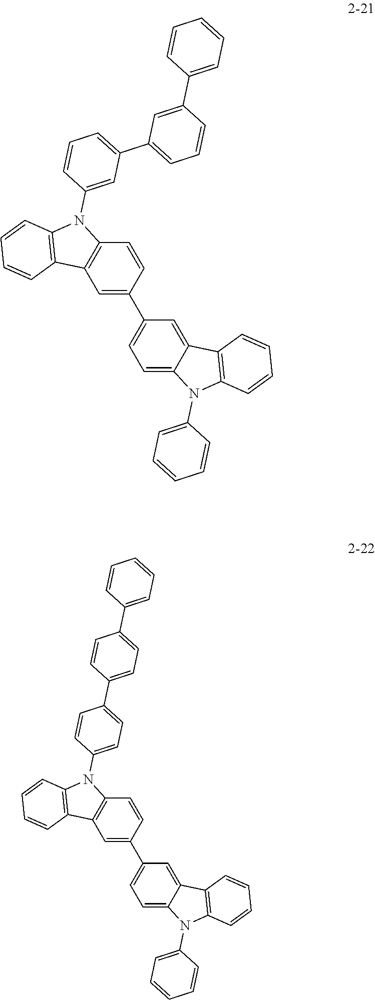 [SMILES: c1ccc(-c2cccc(-c3cccc(-n4c5ccccc5c5cc(-c6ccc7c(c6)c6ccccc6n7-c6ccccc6)ccc54)c3)c2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

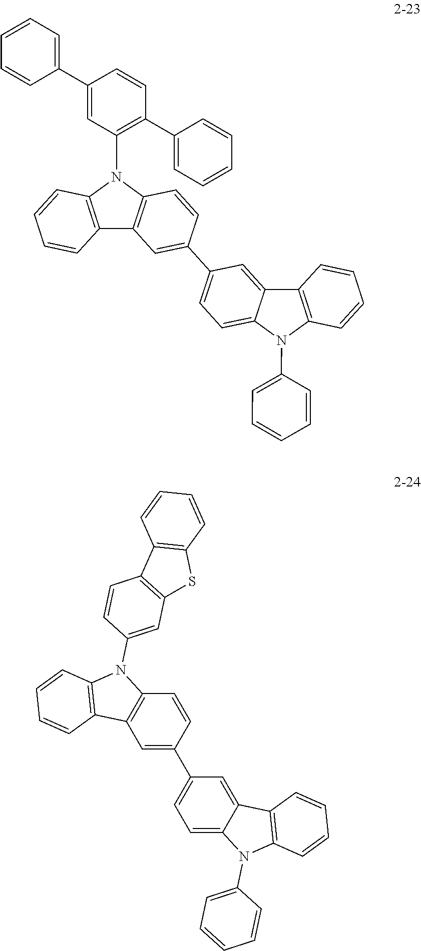 [SMILES: c1ccc(-c2ccc(-c3ccccc3)c(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4ccc5c(c4)sc4ccccc45)ccc32)cc1]

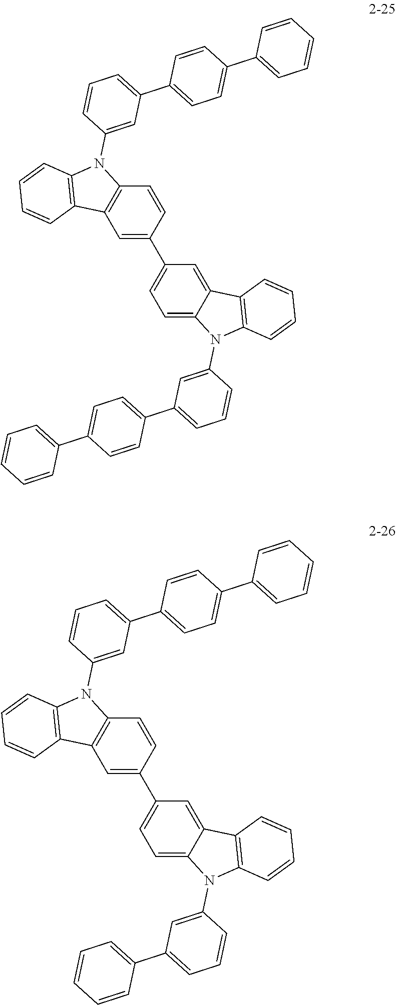 [SMILES: c1ccc(-c2ccc(-c3cccc(-n4c5ccccc5c5cc(-c6ccc7c(c6)c6ccccc6n7-c6cccc(-c7ccc(-c8ccccc8)cc7)c6)ccc54)c3)cc2)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1]

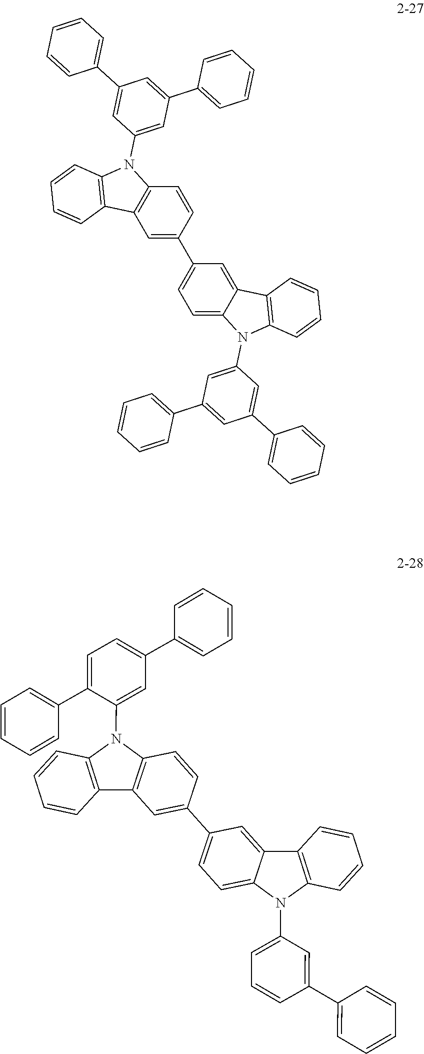 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5ccccc5n6-c5cc(-c6ccccc6)ccc5-c5ccccc5)ccc43)c2)cc1]

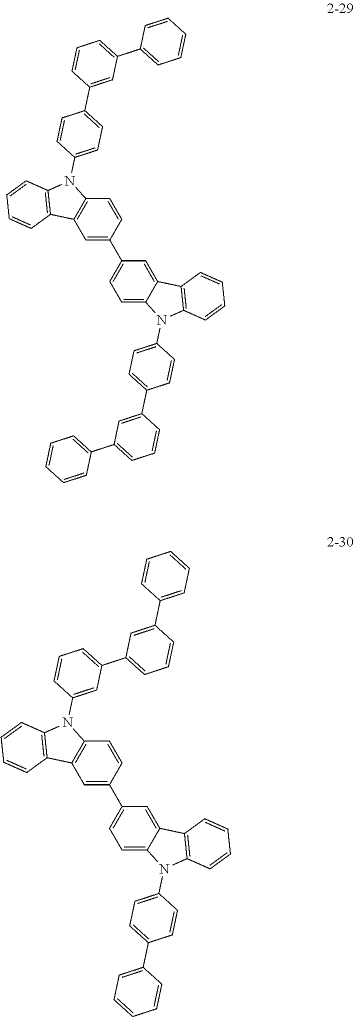 [SMILES: c1ccc(-c2cccc(-c3ccc(-n4c5ccccc5c5cc(-c6ccc7c(c6)c6ccccc6n7-c6ccc(-c7cccc(-c8ccccc8)c7)cc6)ccc54)cc3)c2)cc1 ; c1ccc(-c2ccc(-n3c4ccccc4c4cc(-c5ccc6c(c5)c5ccccc5n6-c5cccc(-c6cccc(-c7ccccc7)c6)c5)ccc43)cc2)cc1]

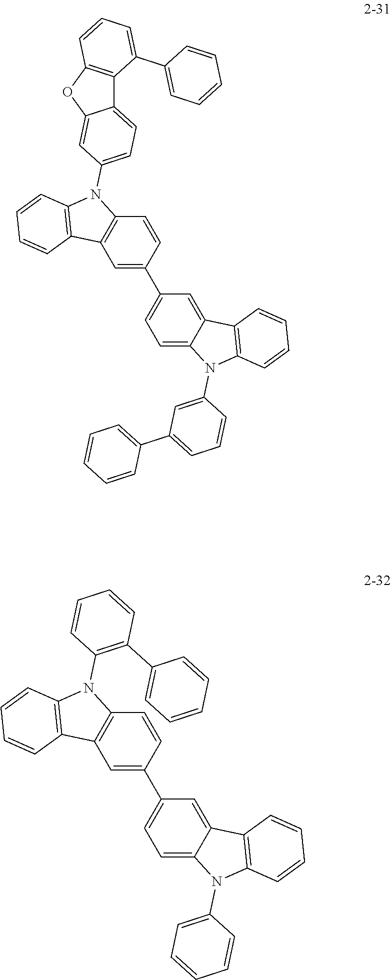 [SMILES: c1ccc(-c2cccc(-n3c4ccccc4c4cc(-c5ccc6c(c5)c5ccccc5n6-c5ccc6c(c5)oc5cccc(-c7ccccc7)c56)ccc43)c2)cc1 ; c1ccc(-c2ccccc2-n2c3ccccc3c3cc(-c4ccc5c(c4)c4ccccc4n5-c4ccccc4)ccc32)cc1]

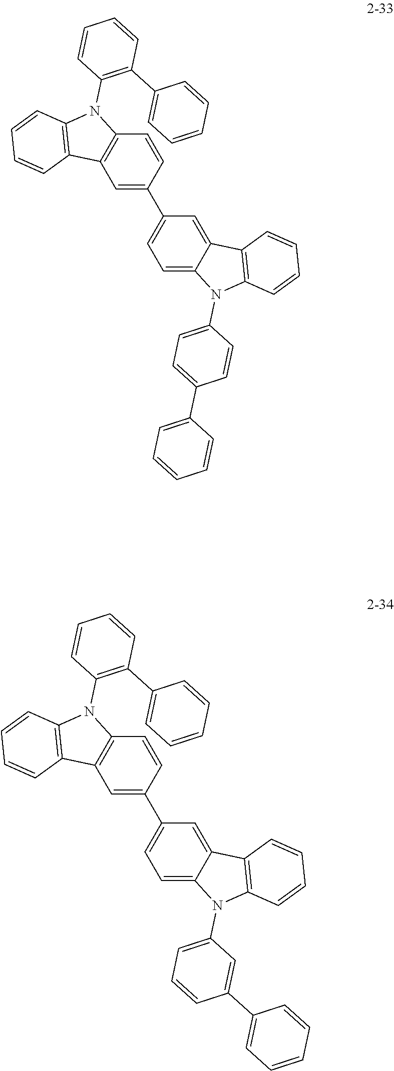 [SMILES: c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc(-n3c4ccccc4c4cc(-c5ccc6c(c5)c5ccccc5n6-c5ccccc5-c5ccccc5)ccc43)c2)cc1]

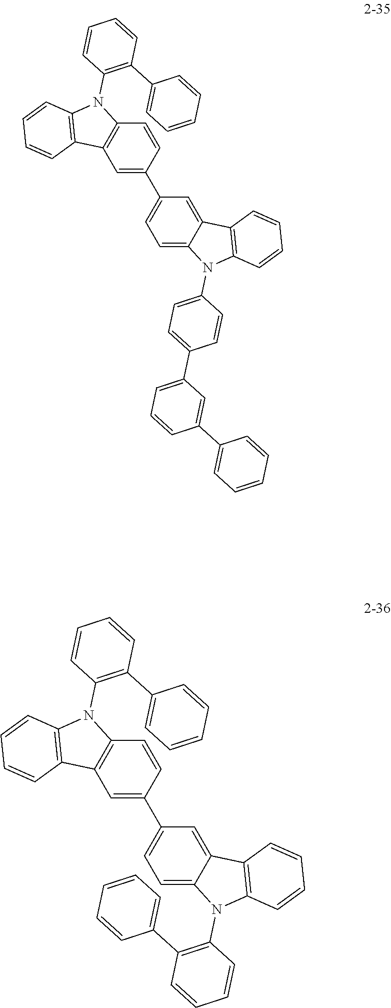 [SMILES: c1ccc(-c2cccc(-c3ccc(-n4c5ccccc5c5cc(-c6ccc7c(c6)c6ccccc6n7-c6ccccc6-c6ccccc6)ccc54)cc3)c2)cc1 ; c1ccc(-c2ccccc2-n2c3ccccc3c3cc(-c4ccc5c(c4)c4ccccc4n5-c4ccccc4-c4ccccc4)ccc32)cc1]

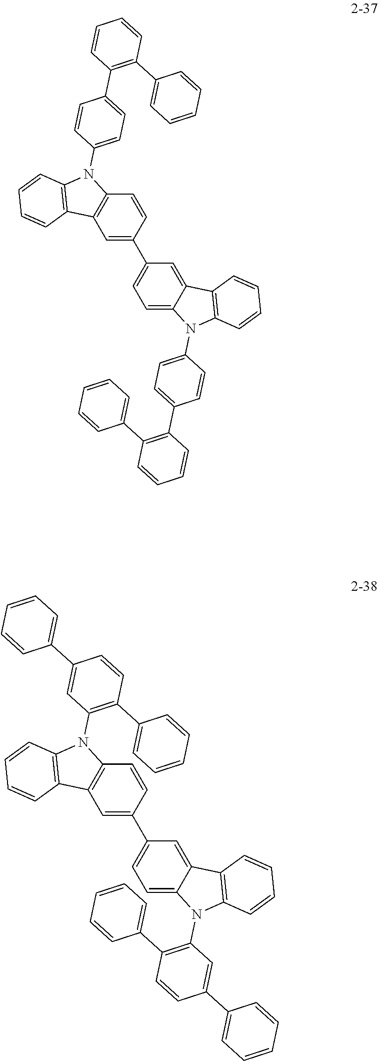 [SMILES: c1ccc(-c2ccccc2-c2ccc(-n3c4ccccc4c4cc(-c5ccc6c(c5)c5ccccc5n6-c5ccc(-c6ccccc6-c6ccccc6)cc5)ccc43)cc2)cc1 ; c1ccc(-c2ccc(-c3ccccc3)c(-n3c4ccccc4c4cc(-c5ccc6c(c5)c5ccccc5n6-c5cc(-c6ccccc6)ccc5-c5ccccc5)ccc43)c2)cc1]

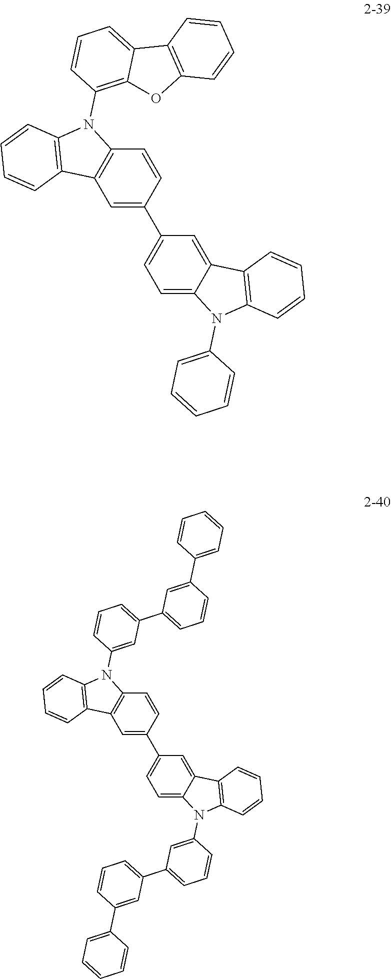 [SMILES: c1ccc(-n2c3ccccc3c3cc(-c4ccc5c(c4)c4ccccc4n5-c4cccc5c4oc4ccccc45)ccc32)cc1 ; c1ccc(-c2cccc(-c3cccc(-n4c5ccccc5c5cc(-c6ccc7c(c6)c6ccccc6n7-c6cccc(-c7cccc(-c8ccccc8)c7)c6)ccc54)c3)c2)cc1]

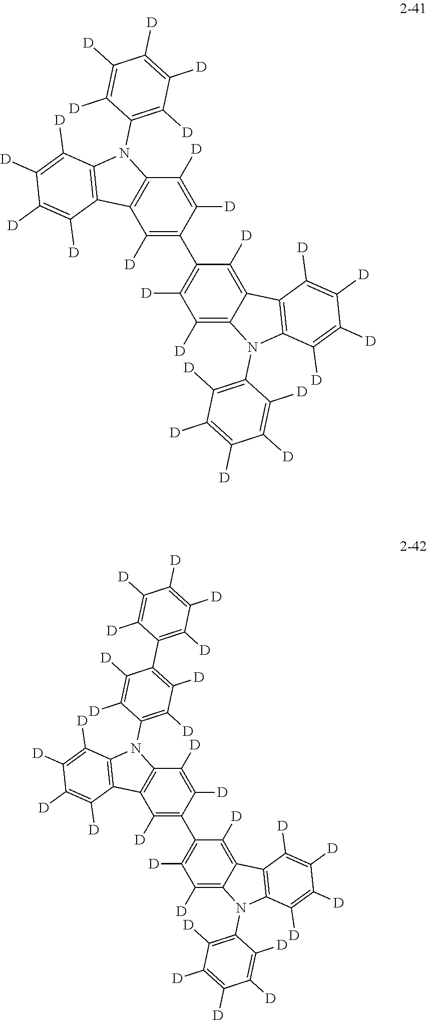 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c([2H])c4[2H])c([2H])c([2H])c32)c([2H])c1[2H] ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c([2H])c2[2H])c([2H])c1[2H]]

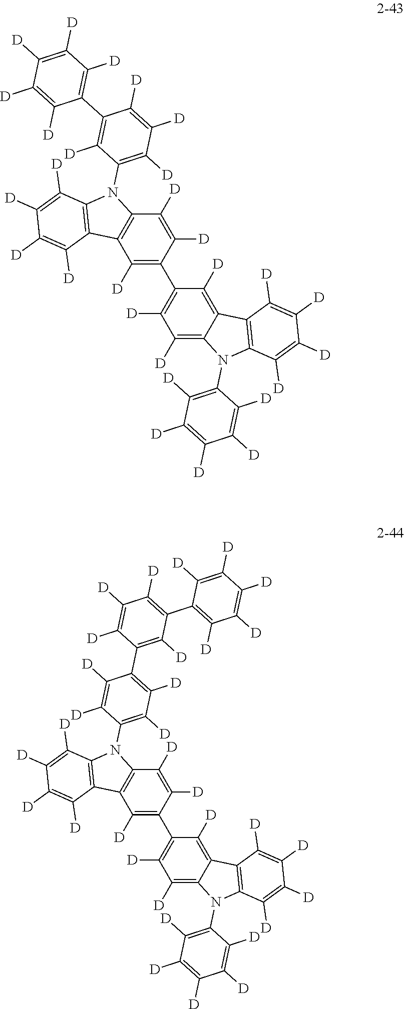 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c2[2H])c([2H])c1[2H] ; [2H]c1c([2H])c([2H])c(-c2c([2H])c([2H])c([2H])c(-c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c([2H])c6[2H])c([2H])c([2H])c54)c([2H])c3[2H])c2[2H])c([2H])c1[2H]]

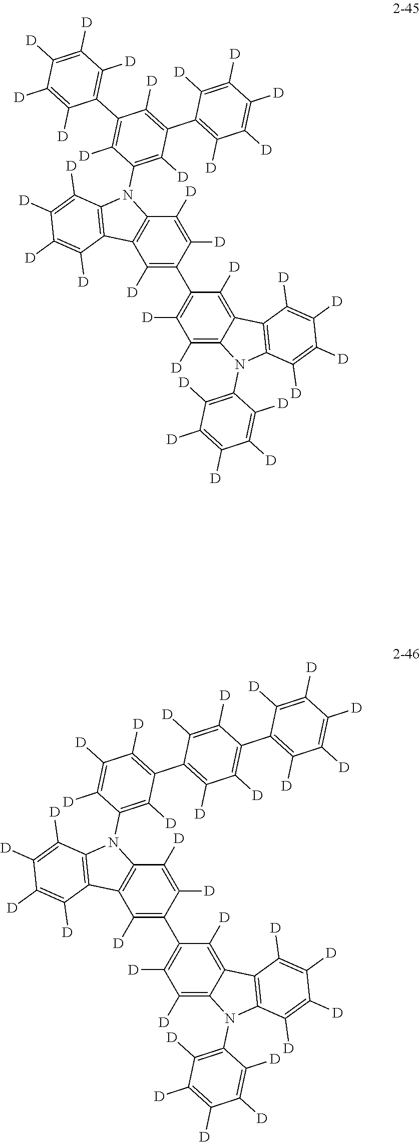

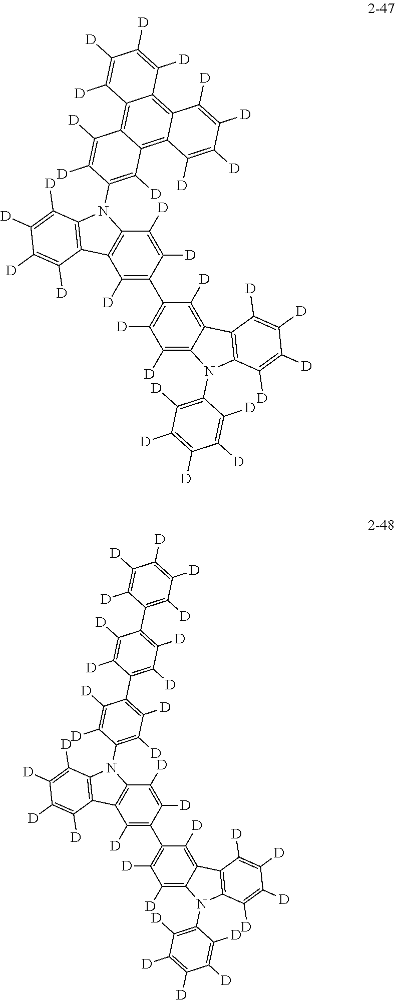

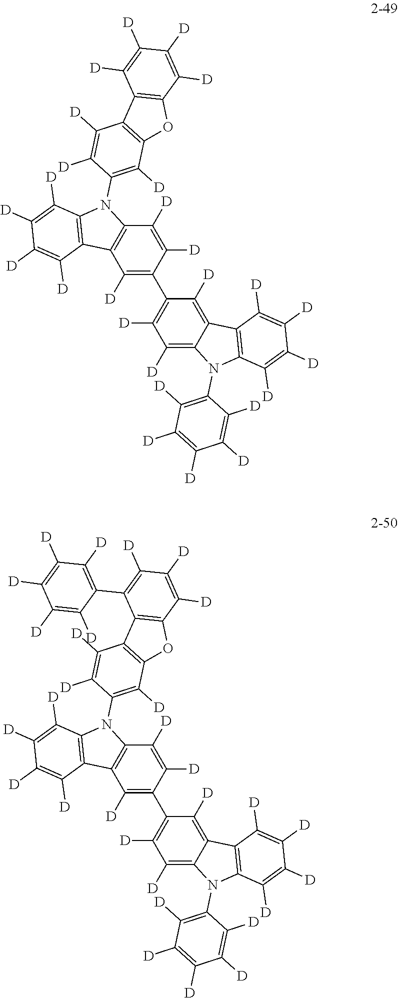 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c(oc6c([2H])c([2H])c([2H])c([2H])c65)c4[2H])c([2H])c([2H])c32)c([2H])c1[2H] ; [2H]c1c([2H])c([2H])c(-c2c([2H])c([2H])c([2H])c3oc4c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7c([2H])c([2H])c([2H])c([2H])c7[2H])c([2H])c([2H])c65)c([2H])c([2H])c4c23)c([2H])c1[2H]]

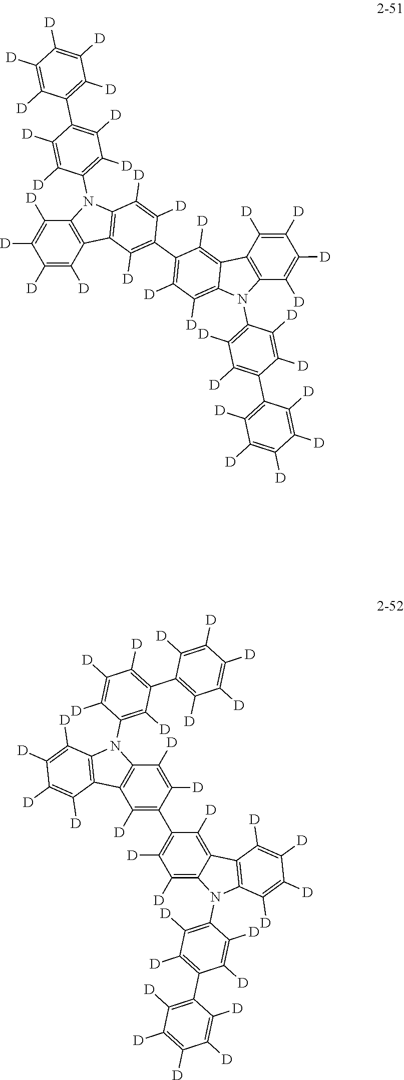

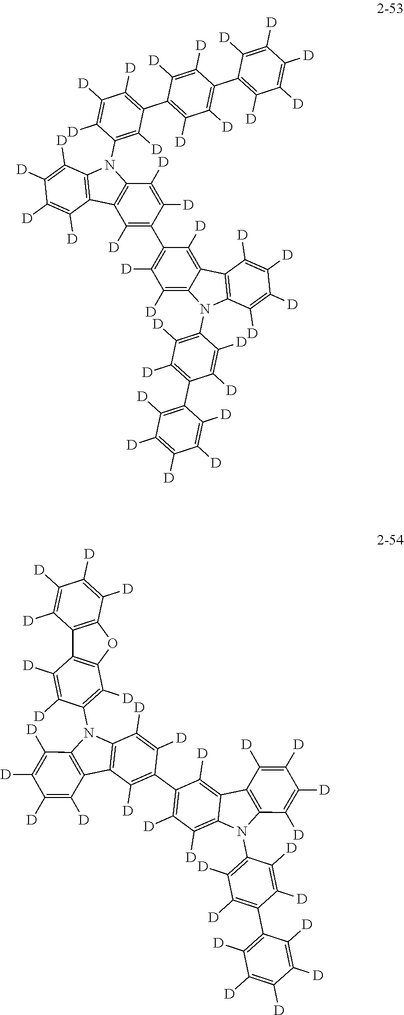

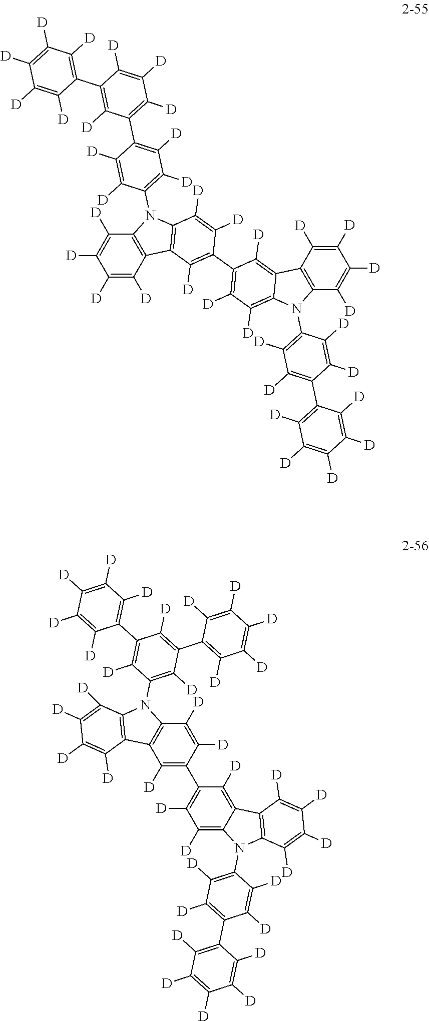

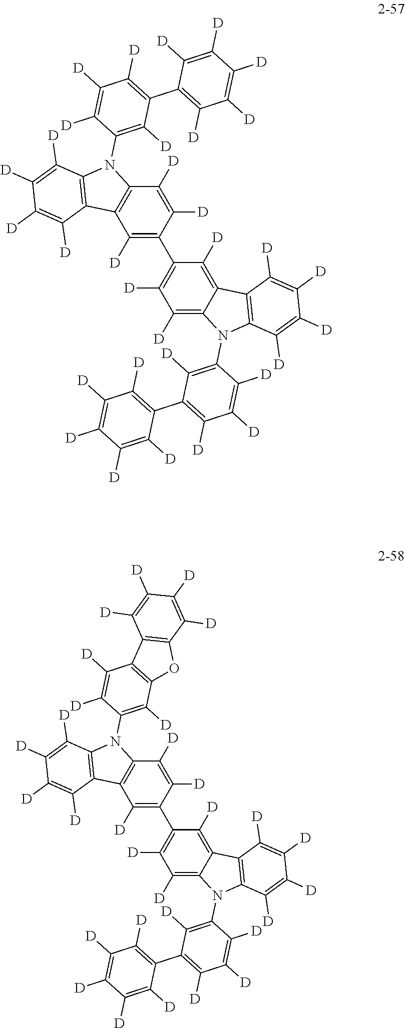

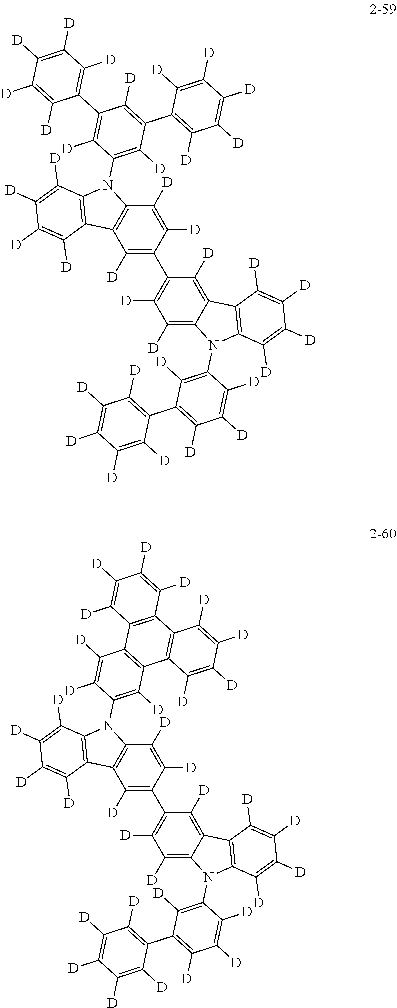

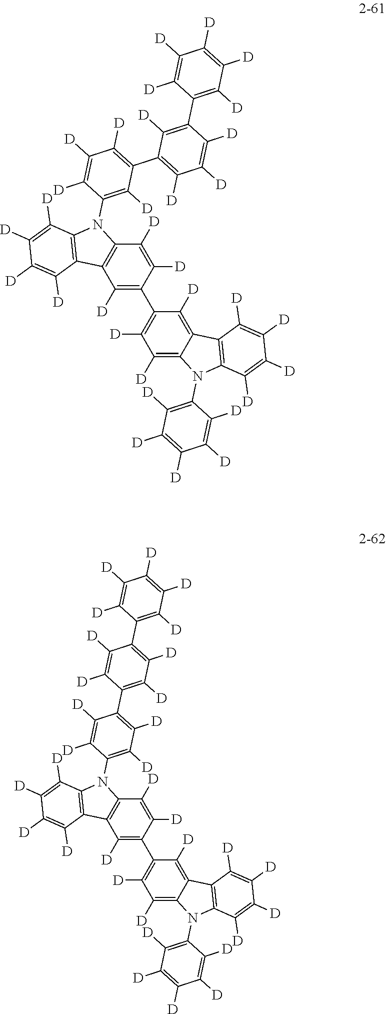

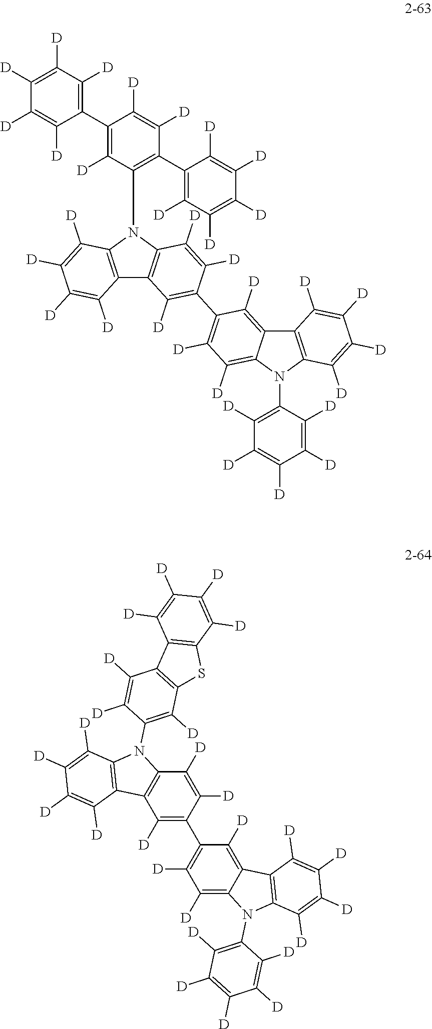 [SMILES: [2H]c1c([2H])c([2H])c(-c2c([2H])c([2H])c(-c3c([2H])c([2H])c([2H])c([2H])c3[2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c2[2H])c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c(sc6c([2H])c([2H])c([2H])c([2H])c65)c4[2H])c([2H])c([2H])c32)c([2H])c1[2H]]

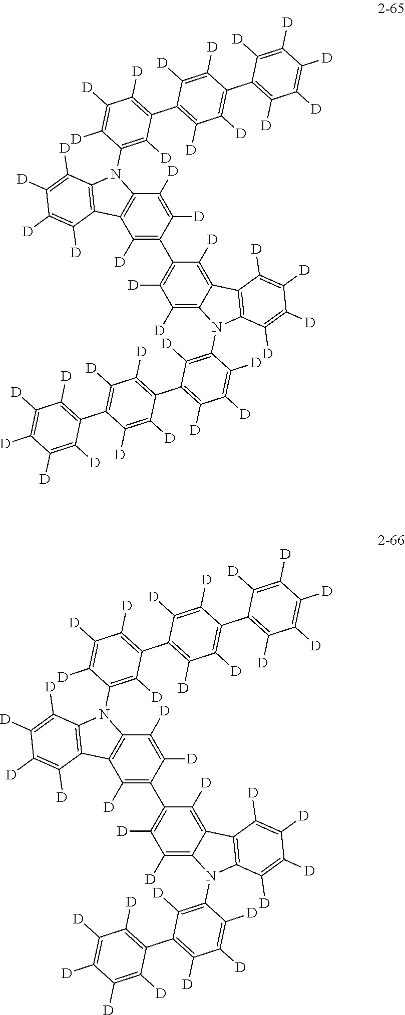

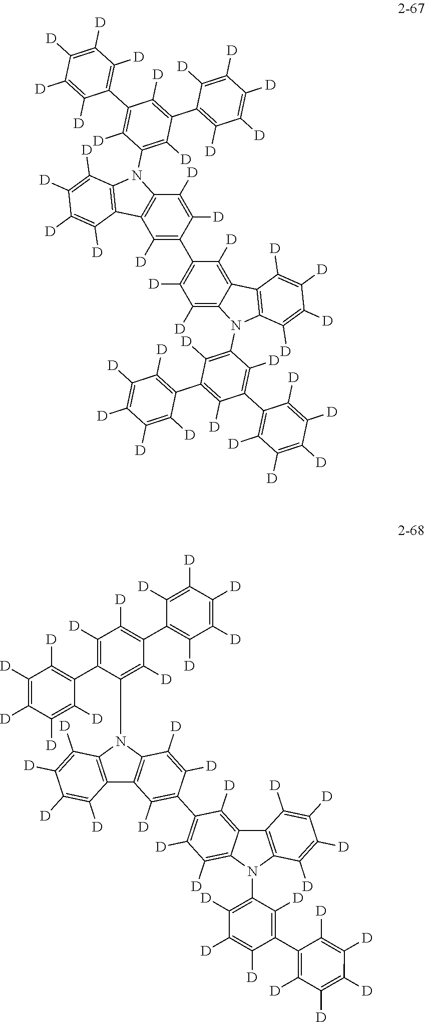

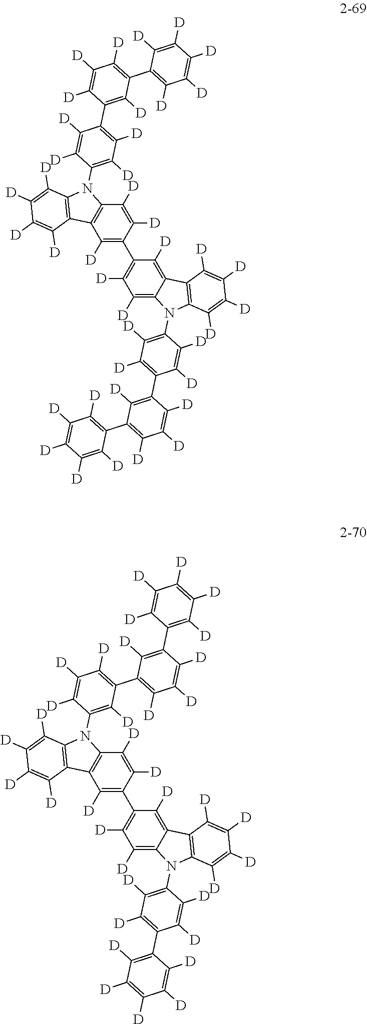

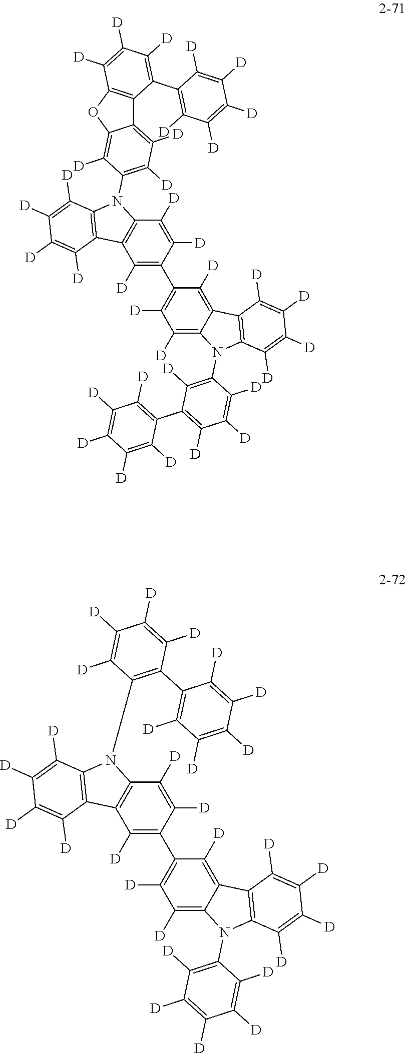

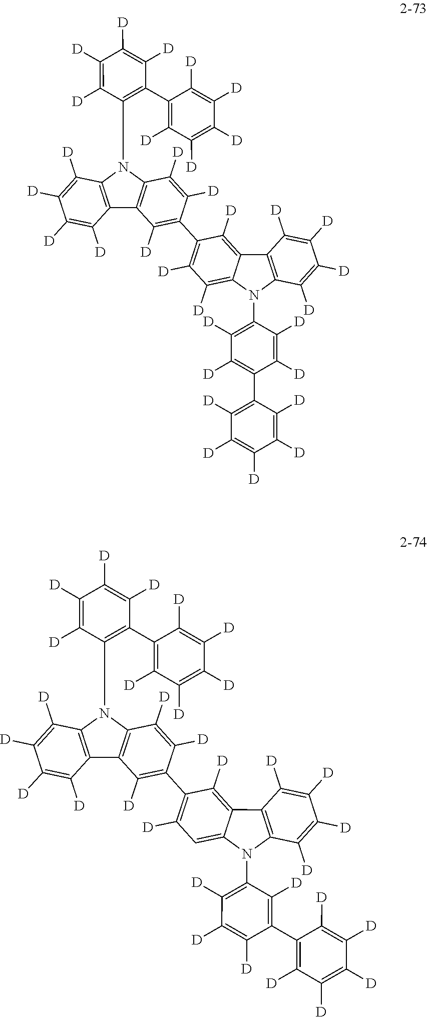

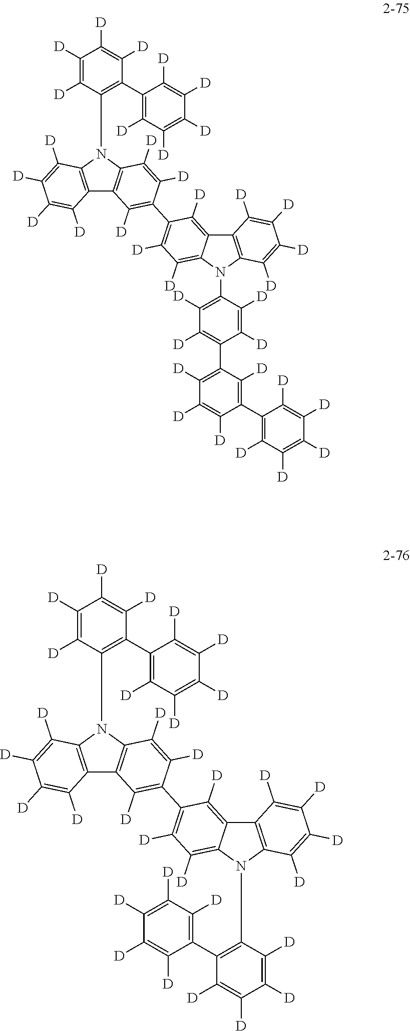

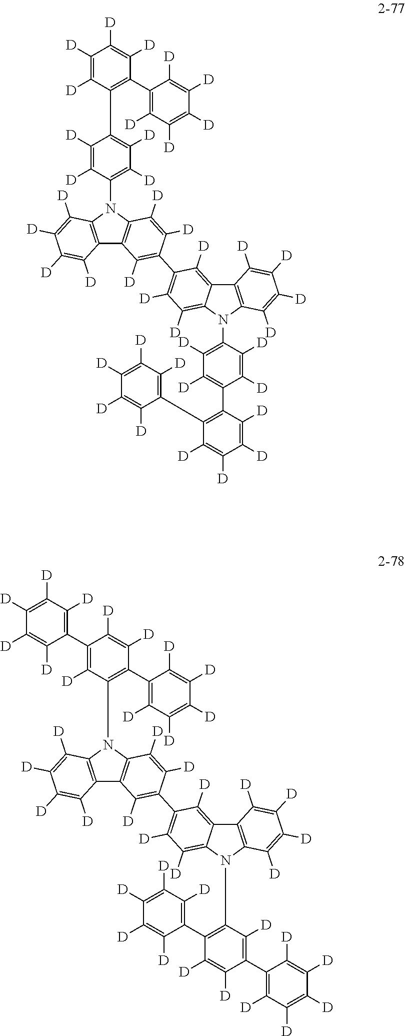

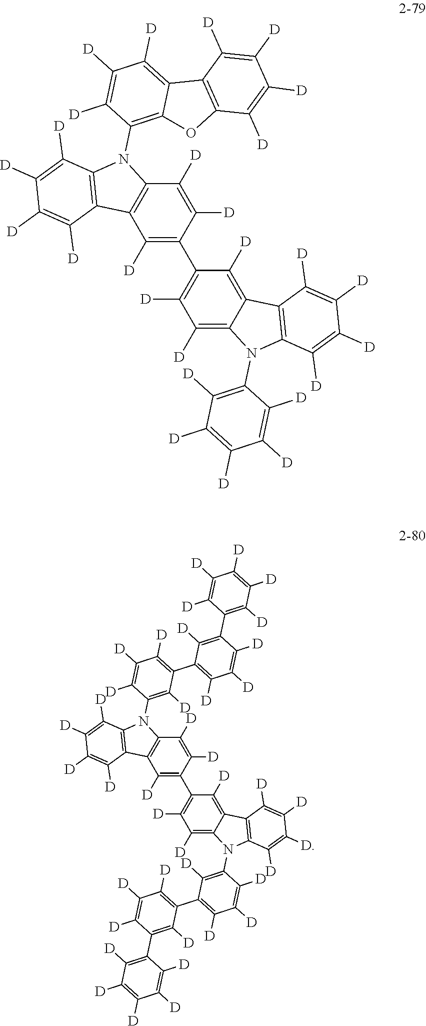

12. The organic light emitting device of claim 10, wherein Chemical Formula 3 is represented by any one of the following compounds:

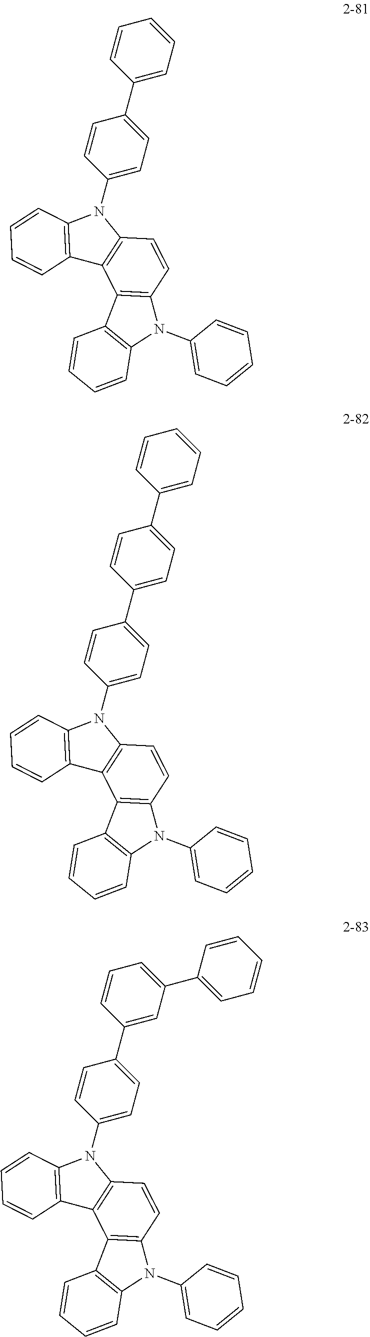

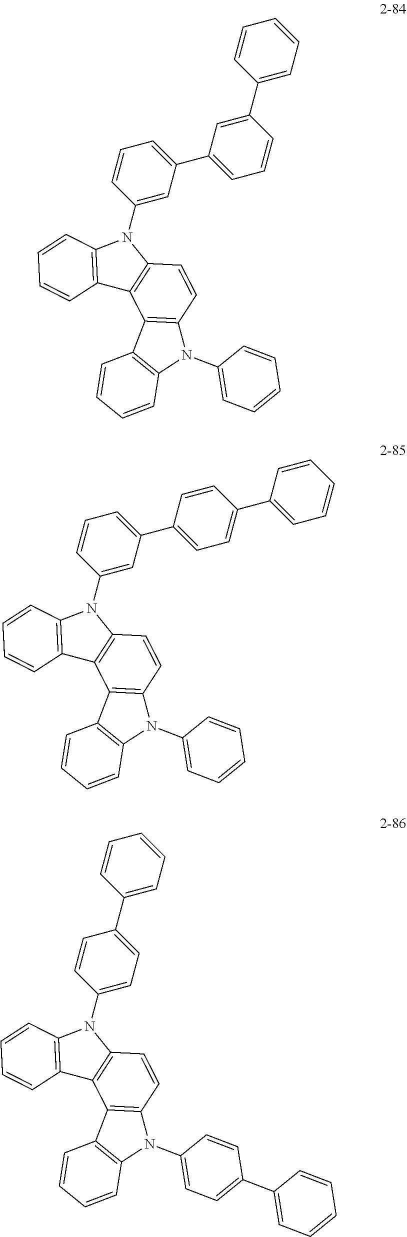 [SMILES: c1ccc(-c2cccc(-c3cccc(-n4c5ccccc5c5c6c7ccccc7n(-c7ccccc7)c6ccc54)c3)c2)cc1 ; c1ccc(-c2ccc(-c3cccc(-n4c5ccccc5c5c6c7ccccc7n(-c7ccccc7)c6ccc54)c3)cc2)cc1 ; c1ccc(-c2ccc(-n3c4ccccc4c4c5c6ccccc6n(-c6ccc(-c7ccccc7)cc6)c5ccc43)cc2)cc1]

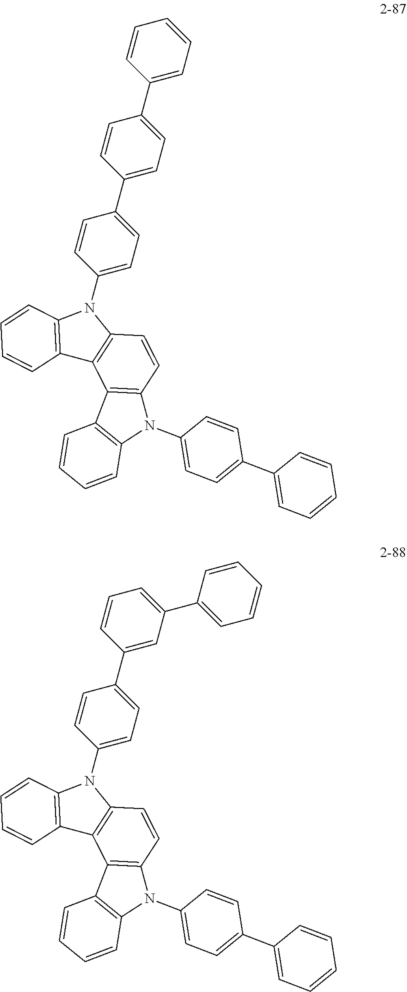 [SMILES: c1ccc(-c2ccc(-c3ccc(-n4c5ccccc5c5c6c7ccccc7n(-c7ccc(-c8ccccc8)cc7)c6ccc54)cc3)cc2)cc1 ; c1ccc(-c2ccc(-n3c4ccccc4c4c5c6ccccc6n(-c6ccc(-c7cccc(-c8ccccc8)c7)cc6)c5ccc43)cc2)cc1]

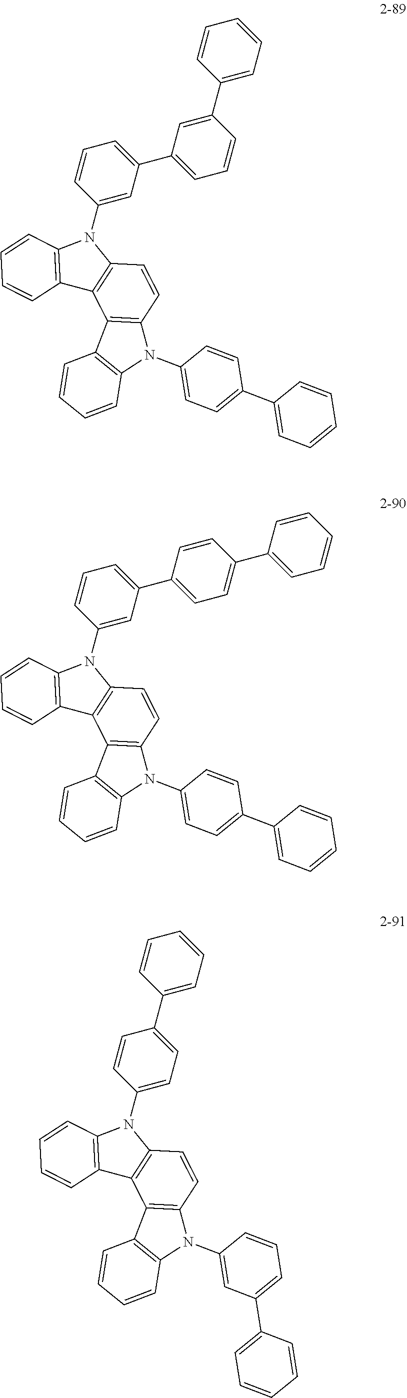 [SMILES: c1ccc(-c2ccc(-n3c4ccccc4c4c5c6ccccc6n(-c6cccc(-c7cccc(-c8ccccc8)c7)c6)c5ccc43)cc2)cc1 ; c1ccc(-c2ccc(-c3cccc(-n4c5ccccc5c5c6c7ccccc7n(-c7ccc(-c8ccccc8)cc7)c6ccc54)c3)cc2)cc1 ; c1ccc(-c2ccc(-n3c4ccccc4c4c5c6ccccc6n(-c6cccc(-c7ccccc7)c6)c5ccc43)cc2)cc1]

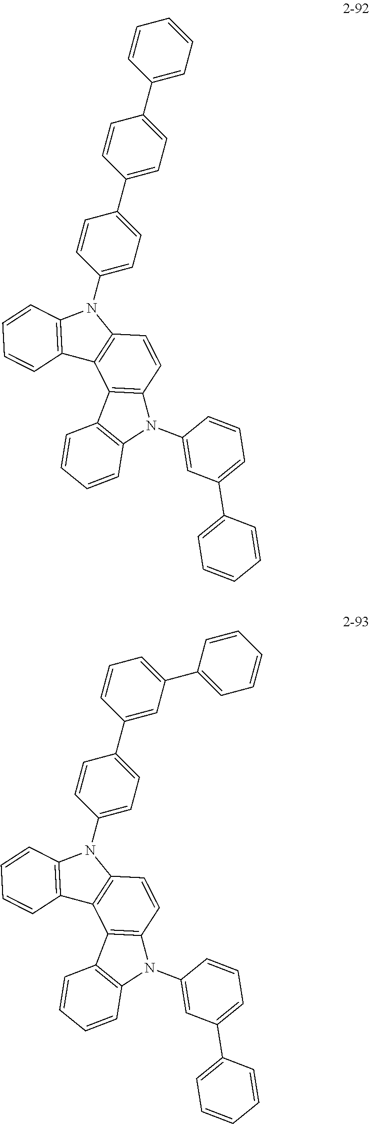 [SMILES: c1ccc(-c2ccc(-c3ccc(-n4c5ccccc5c5c6c7ccccc7n(-c7cccc(-c8ccccc8)c7)c6ccc54)cc3)cc2)cc1 ; c1ccc(-c2cccc(-c3ccc(-n4c5ccccc5c5c6c7ccccc7n(-c7cccc(-c8ccccc8)c7)c6ccc54)cc3)c2)cc1]

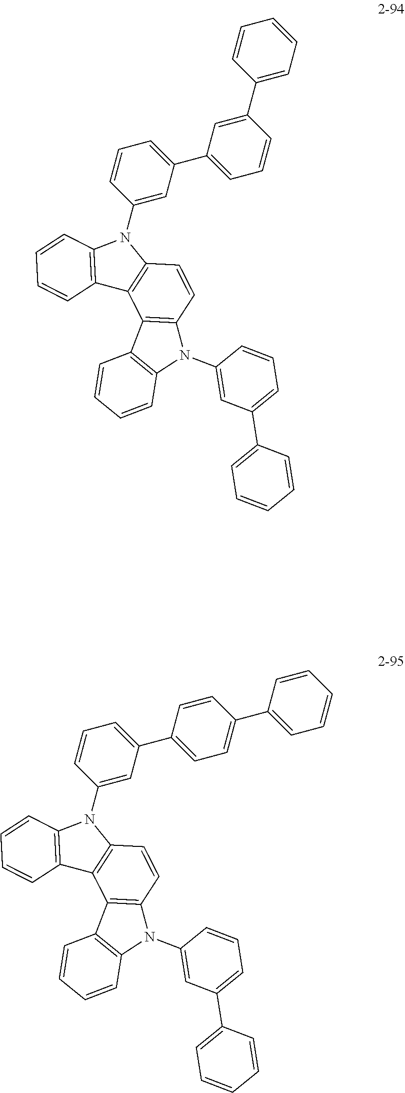 [SMILES: c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2ccc(-c3cccc(-n4c5ccccc5c5c6c7ccccc7n(-c7cccc(-c8ccccc8)c7)c6ccc54)c3)cc2)cc1]

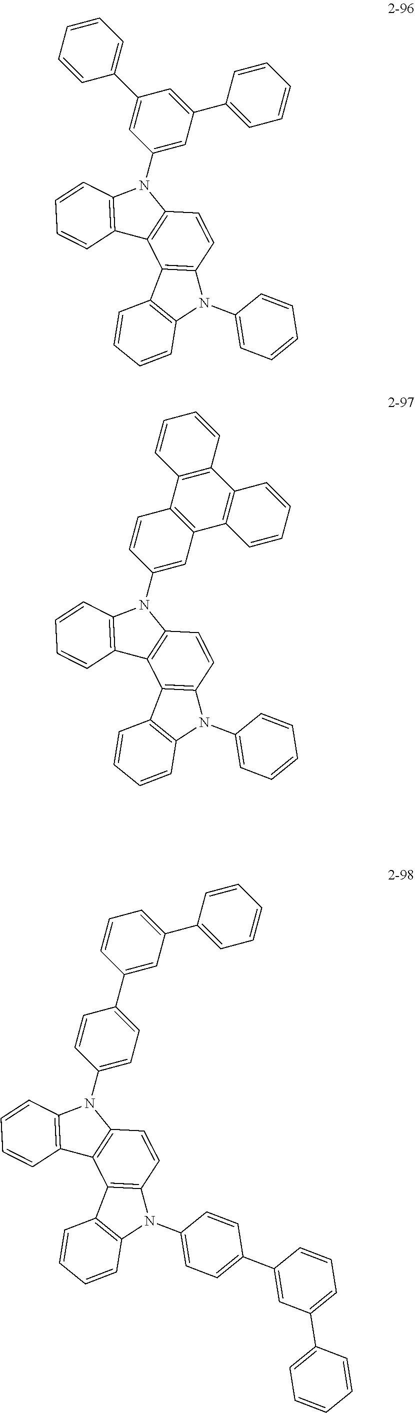 [SMILES: c1ccc(-c2cc(-c3ccccc3)cc(-n3c4ccccc4c4c5c6ccccc6n(-c6ccccc6)c5ccc43)c2)cc1 ; c1ccc(-n2c3ccccc3c3c4c5ccccc5n(-c5ccc6c7ccccc7c7ccccc7c6c5)c4ccc32)cc1 ; c1ccc(-c2cccc(-c3ccc(-n4c5ccccc5c5c6c7ccccc7n(-c7ccc(-c8cccc(-c9ccccc9)c8)cc7)c6ccc54)cc3)c2)cc1]

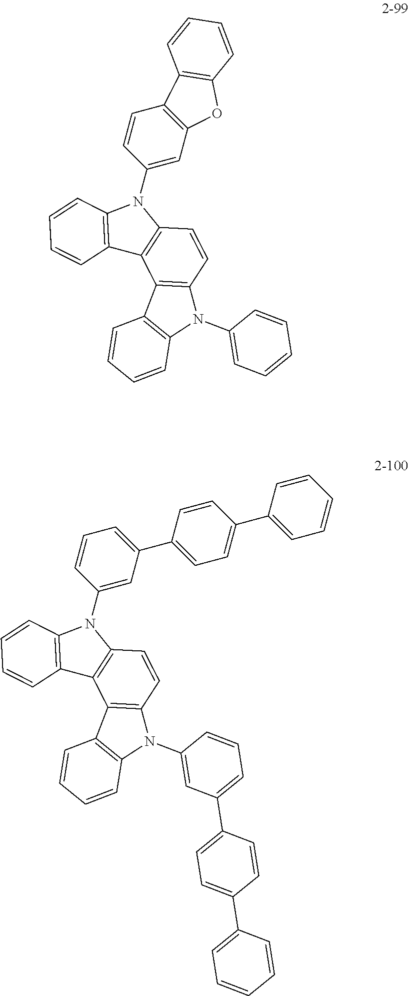 [SMILES: c1ccc(-n2c3ccccc3c3c4c5ccccc5n(-c5ccc6c(c5)oc5ccccc56)c4ccc32)cc1 ; c1ccc(-c2ccc(-c3cccc(-n4c5ccccc5c5c6c7ccccc7n(-c7cccc(-c8ccc(-c9ccccc9)cc8)c7)c6ccc54)c3)cc2)cc1]

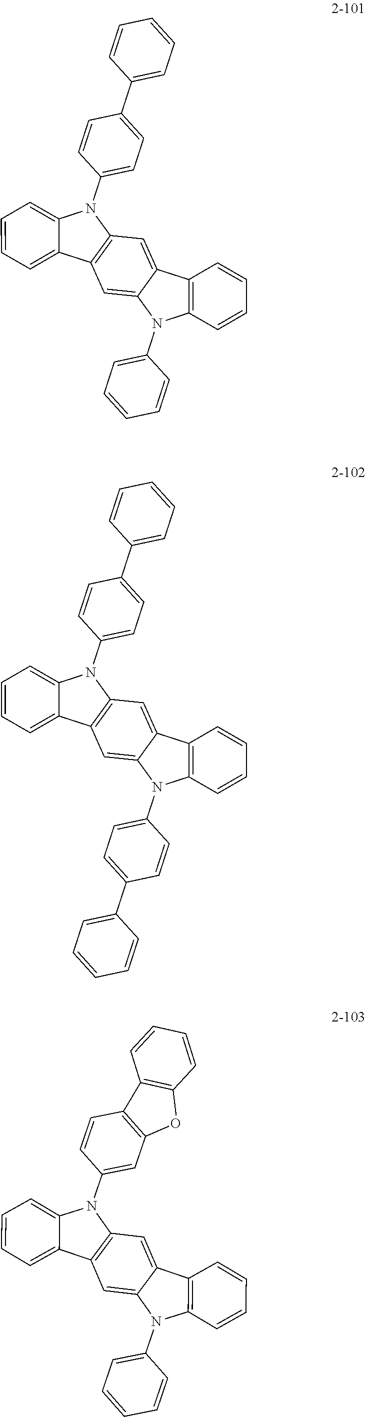 [SMILES: c1ccc(-c2ccc(-n3c4ccccc4c4cc5c(cc43)c3ccccc3n5-c3ccccc3)cc2)cc1 ; c1ccc(-c2ccc(-n3c4ccccc4c4cc5c(cc43)c3ccccc3n5-c3ccc(-c4ccccc4)cc3)cc2)cc1 ; c1ccc(-n2c3ccccc3c3cc4c(cc32)c2ccccc2n4-c2ccc3c(c2)oc2ccccc23)cc1]

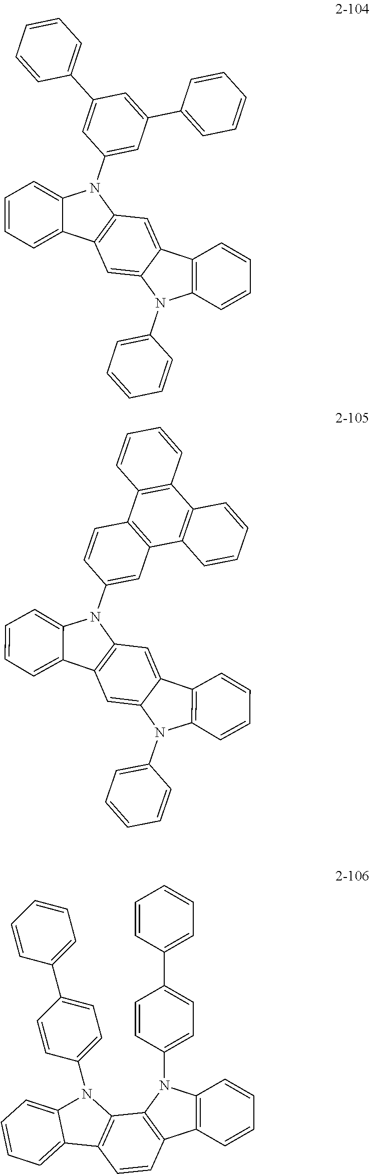

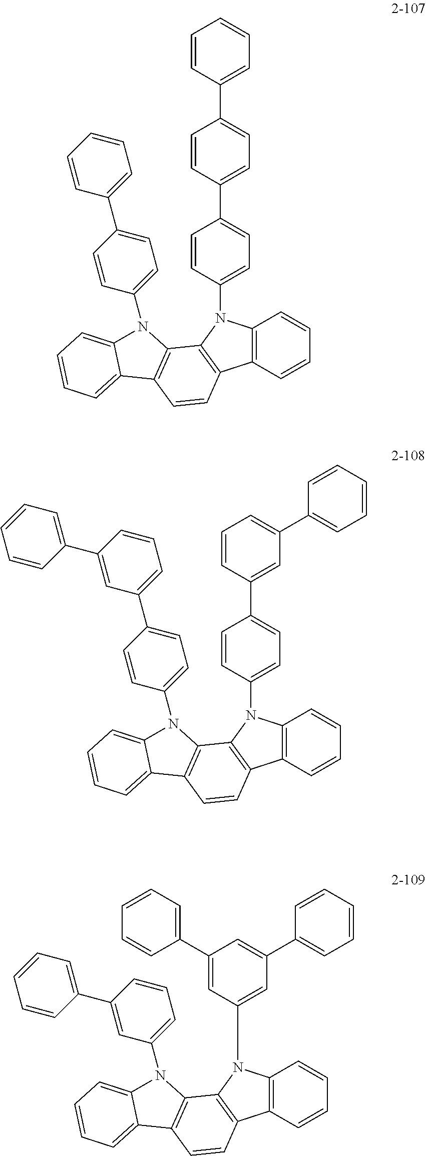 [SMILES: c1ccc(-c2ccc(-c3ccc(-n4c5ccccc5c5ccc6c7ccccc7n(-c7ccc(-c8ccccc8)cc7)c6c54)cc3)cc2)cc1 ; c1ccc(-c2cccc(-c3ccc(-n4c5ccccc5c5ccc6c7ccccc7n(-c7ccc(-c8cccc(-c9ccccc9)c8)cc7)c6c54)cc3)c2)cc1 ; c1ccc(-c2cccc(-n3c4ccccc4c4ccc5c6ccccc6n(-c6cc(-c7ccccc7)cc(-c7ccccc7)c6)c5c43)c2)cc1]

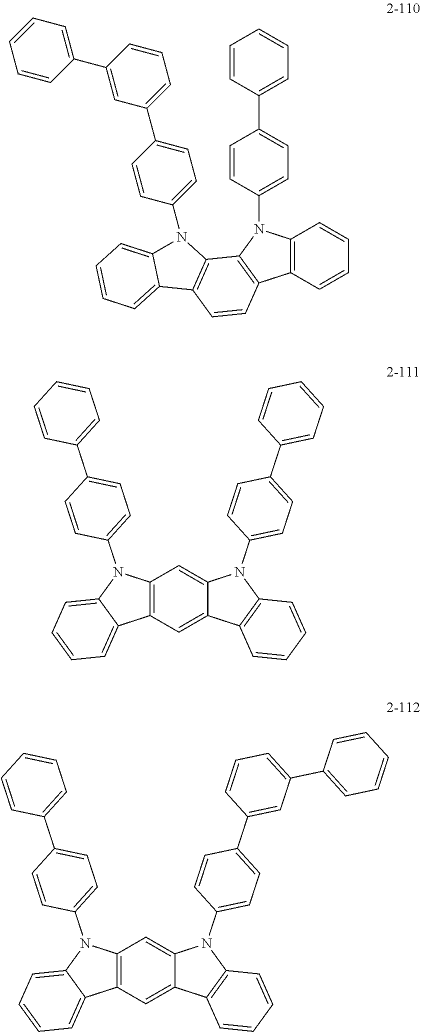 [SMILES: c1ccc(-c2ccc(-n3c4ccccc4c4ccc5c6ccccc6n(-c6ccc(-c7cccc(-c8ccccc8)c7)cc6)c5c43)cc2)cc1 ; c1ccc(-c2ccc(-n3c4ccccc4c4cc5c6ccccc6n(-c6ccc(-c7ccccc7)cc6)c5cc43)cc2)cc1 ; c1ccc(-c2ccc(-n3c4ccccc4c4cc5c6ccccc6n(-c6ccc(-c7cccc(-c8ccccc8)c7)cc6)c5cc43)cc2)cc1]

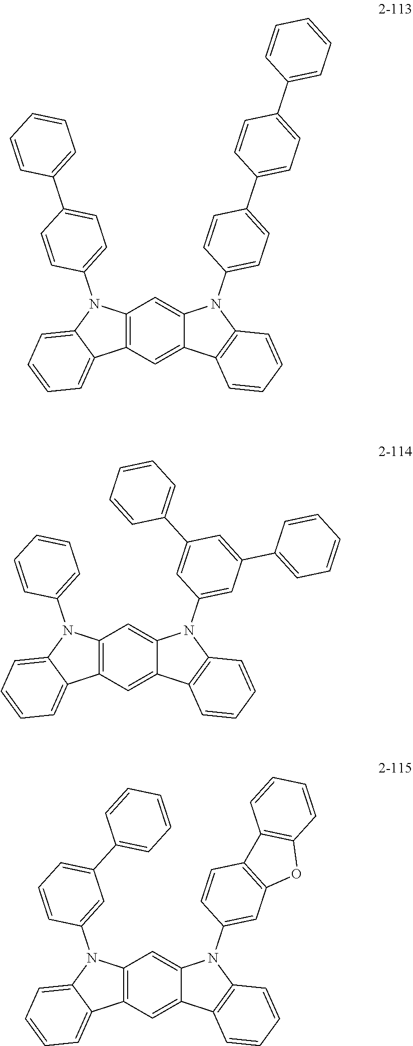 [SMILES: c1ccc(-c2ccc(-c3ccc(-n4c5ccccc5c5cc6c7ccccc7n(-c7ccc(-c8ccccc8)cc7)c6cc54)cc3)cc2)cc1 ; c1ccc(-c2cc(-c3ccccc3)cc(-n3c4ccccc4c4cc5c6ccccc6n(-c6ccccc6)c5cc43)c2)cc1 ; c1ccc(-c2cccc(-n3c4ccccc4c4cc5c6ccccc6n(-c6ccc7c(c6)oc6ccccc67)c5cc43)c2)cc1]

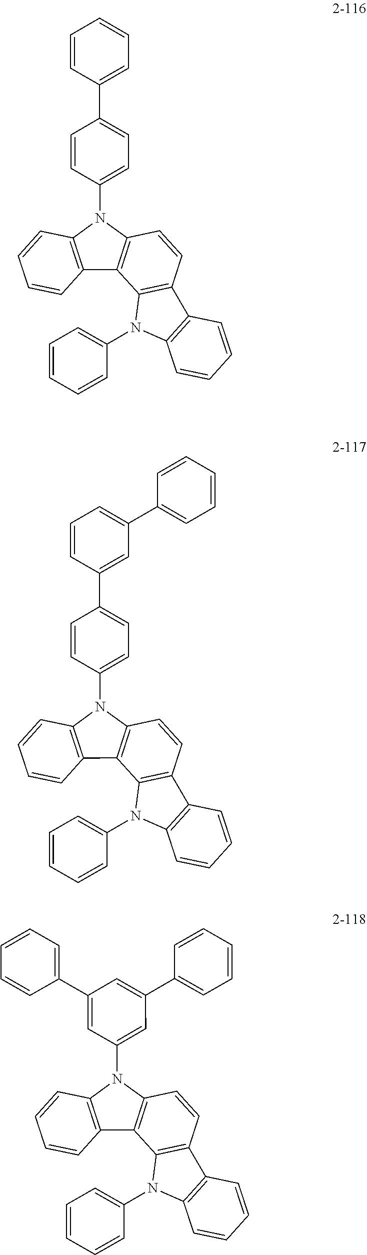 [SMILES: c1ccc(-c2ccc(-n3c4ccccc4c4c3ccc3c5ccccc5n(-c5ccccc5)c34)cc2)cc1 ; c1ccc(-c2cccc(-c3ccc(-n4c5ccccc5c5c4ccc4c6ccccc6n(-c6ccccc6)c45)cc3)c2)cc1 ; c1ccc(-c2cc(-c3ccccc3)cc(-n3c4ccccc4c4c3ccc3c5ccccc5n(-c5ccccc5)c34)c2)cc1]

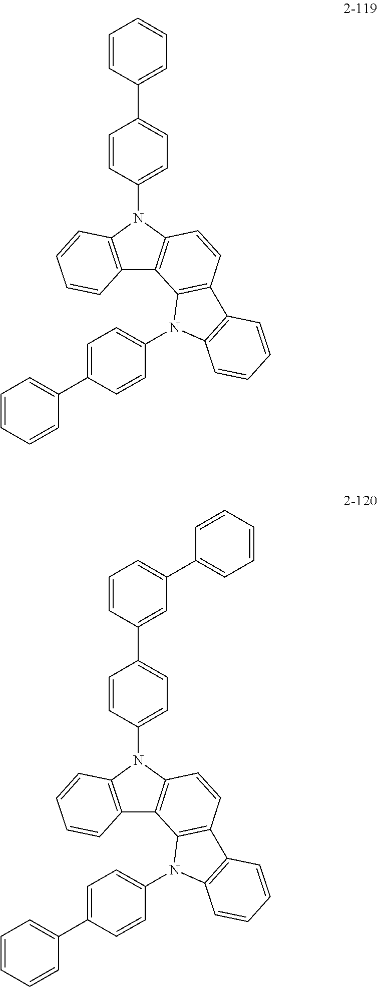 [SMILES: c1ccc(-c2ccc(-n3c4ccccc4c4c3ccc3c5ccccc5n(-c5ccc(-c6ccccc6)cc5)c34)cc2)cc1 ; c1ccc(-c2ccc(-n3c4ccccc4c4ccc5c(c6ccccc6n5-c5ccc(-c6cccc(-c7ccccc7)c6)cc5)c43)cc2)cc1]

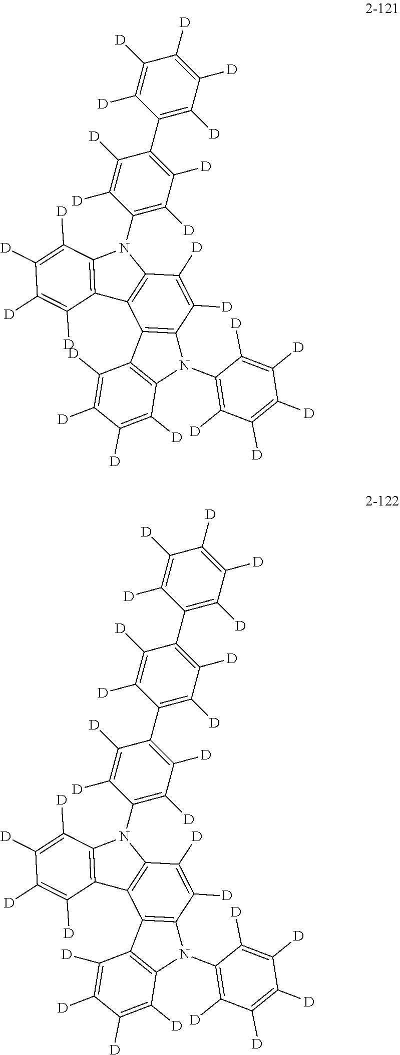 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c([2H])c([2H])c6[2H])c5c([2H])c([2H])c43)c([2H])c2[2H])c([2H])c1[2H] ; [2H]c1c([2H])c([2H])c(-c2c([2H])c([2H])c(-c3c([2H])c([2H])c(-n4c5c([2H])c([2H])c([2H])c([2H])c5c5c6c7c([2H])c([2H])c([2H])c([2H])c7n(-c7c([2H])c([2H])c([2H])c([2H])c7[2H])c6c([2H])c([2H])c54)c([2H])c3[2H])c([2H])c2[2H])c([2H])c1[2H]]

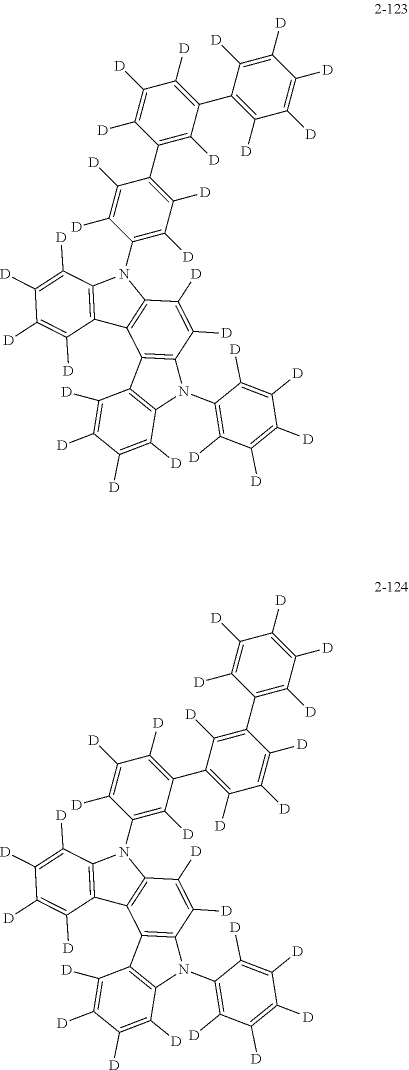 [SMILES: [2H]c1c([2H])c([2H])c(-c2c([2H])c([2H])c([2H])c(-c3c([2H])c([2H])c(-n4c5c([2H])c([2H])c([2H])c([2H])c5c5c6c7c([2H])c([2H])c([2H])c([2H])c7n(-c7c([2H])c([2H])c([2H])c([2H])c7[2H])c6c([2H])c([2H])c54)c([2H])c3[2H])c2[2H])c([2H])c1[2H] ; [2H]c1c([2H])c([2H])c(-c2c([2H])c([2H])c([2H])c(-c3c([2H])c([2H])c([2H])c(-n4c5c([2H])c([2H])c([2H])c([2H])c5c5c6c7c([2H])c([2H])c([2H])c([2H])c7n(-c7c([2H])c([2H])c([2H])c([2H])c7[2H])c6c([2H])c([2H])c54)c3[2H])c2[2H])c([2H])c1[2H]]

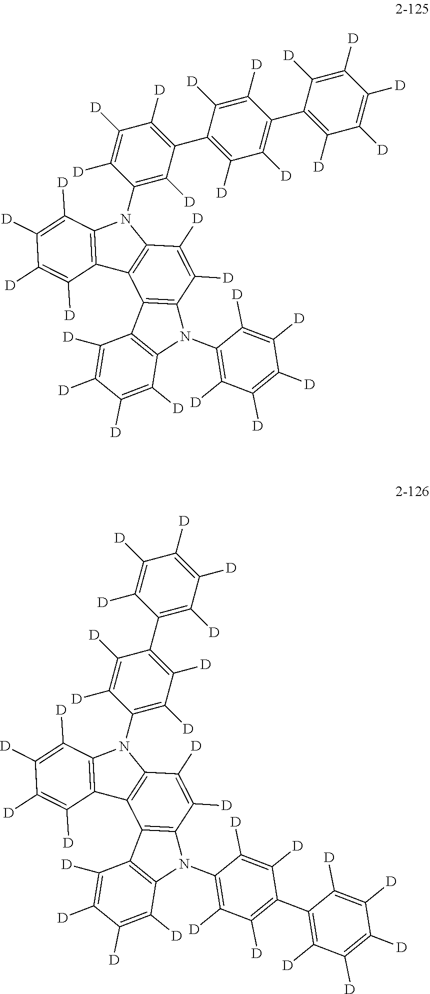 [SMILES: [2H]c1c([2H])c([2H])c(-c2c([2H])c([2H])c(-c3c([2H])c([2H])c([2H])c(-n4c5c([2H])c([2H])c([2H])c([2H])c5c5c6c7c([2H])c([2H])c([2H])c([2H])c7n(-c7c([2H])c([2H])c([2H])c([2H])c7[2H])c6c([2H])c([2H])c54)c3[2H])c([2H])c2[2H])c([2H])c1[2H] ; [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c(-c7c([2H])c([2H])c([2H])c([2H])c7[2H])c([2H])c6[2H])c5c([2H])c([2H])c43)c([2H])c2[2H])c([2H])c1[2H]]

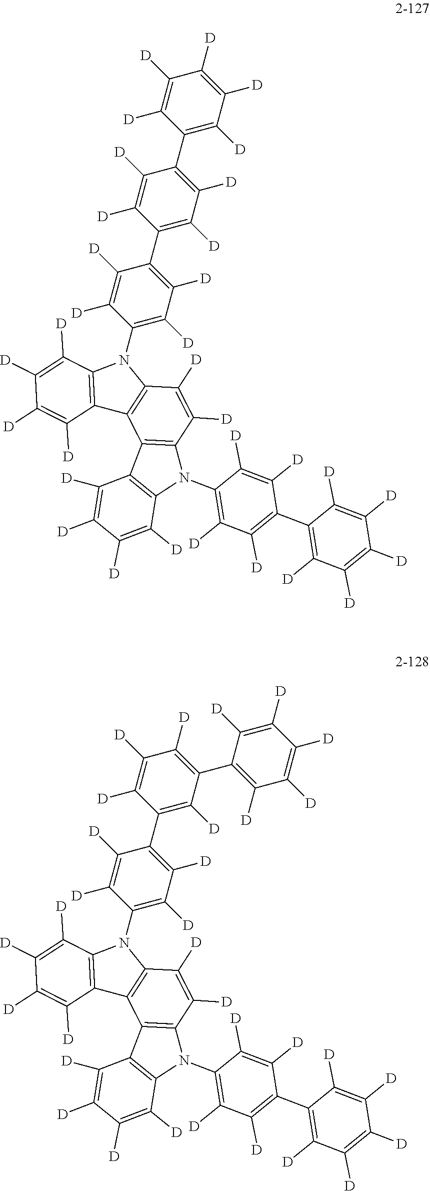

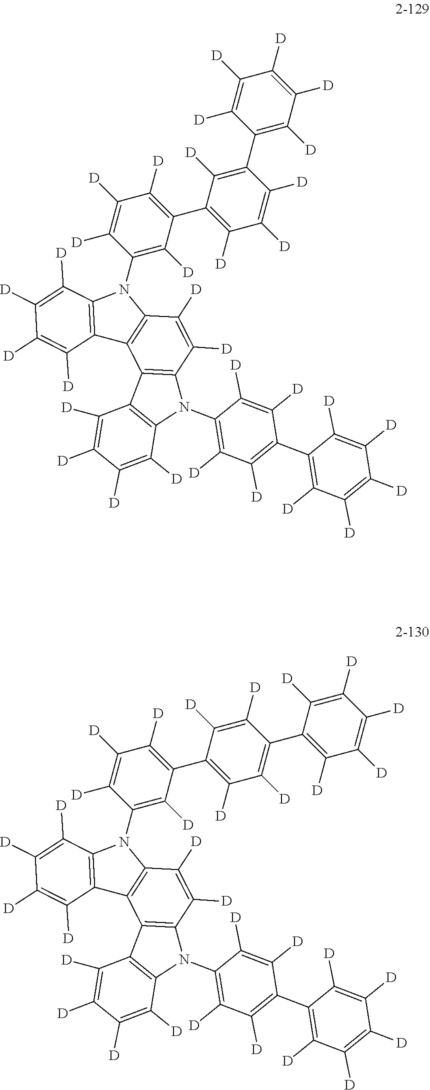

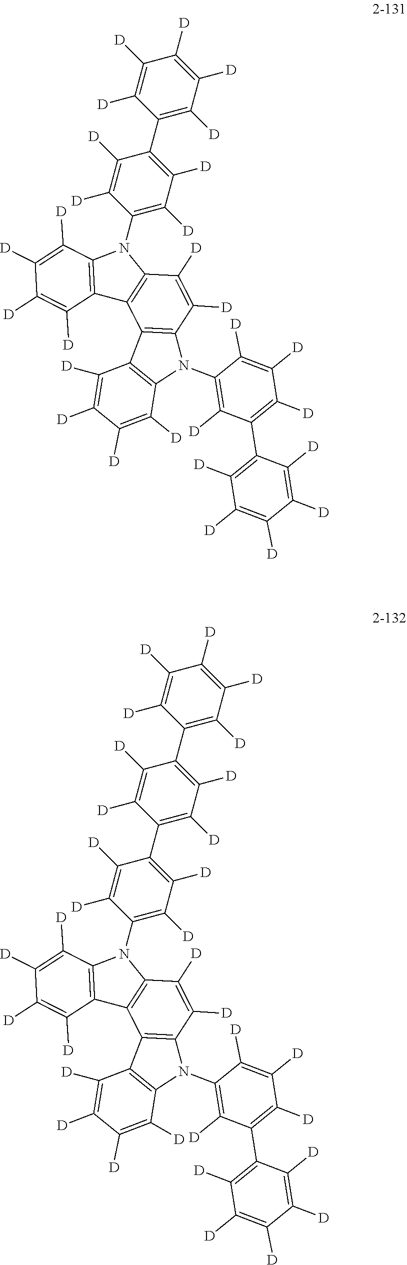 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c([2H])c43)c([2H])c2[2H])c([2H])c1[2H] ; [2H]c1c([2H])c([2H])c(-c2c([2H])c([2H])c(-c3c([2H])c([2H])c(-n4c5c([2H])c([2H])c([2H])c([2H])c5c5c6c7c([2H])c([2H])c([2H])c([2H])c7n(-c7c([2H])c([2H])c([2H])c(-c8c([2H])c([2H])c([2H])c([2H])c8[2H])c7[2H])c6c([2H])c([2H])c54)c([2H])c3[2H])c([2H])c2[2H])c([2H])c1[2H]]

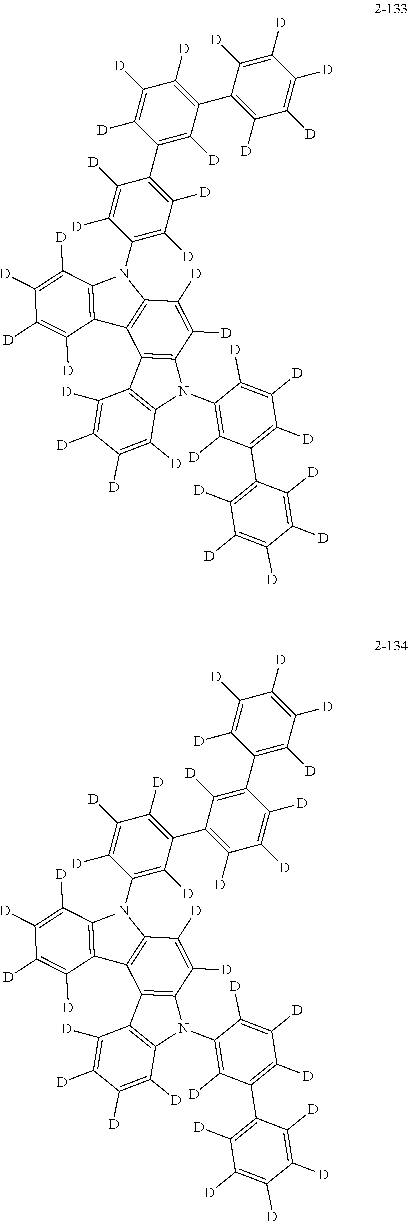

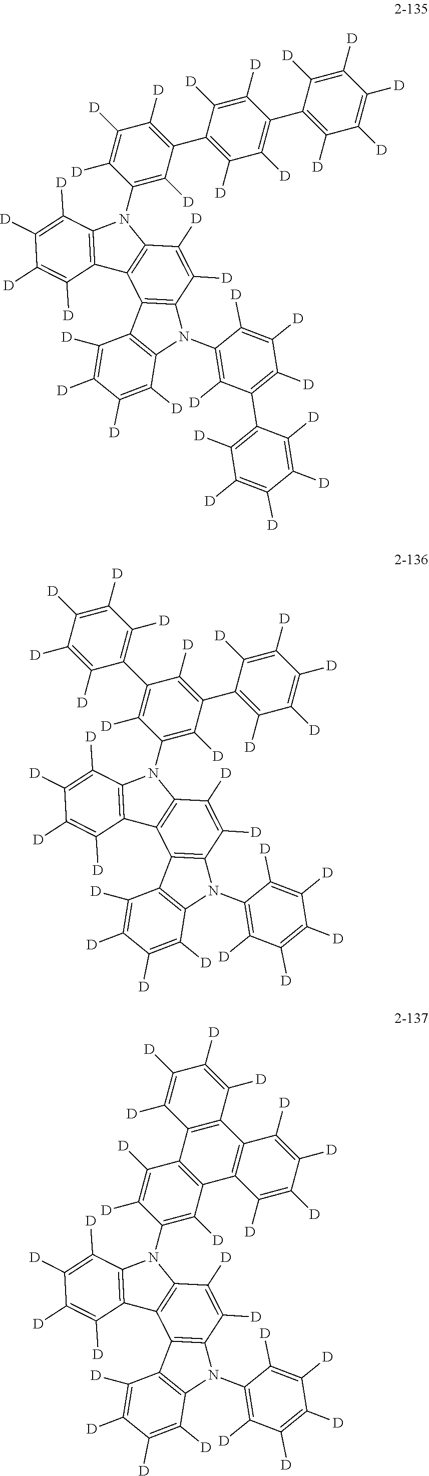

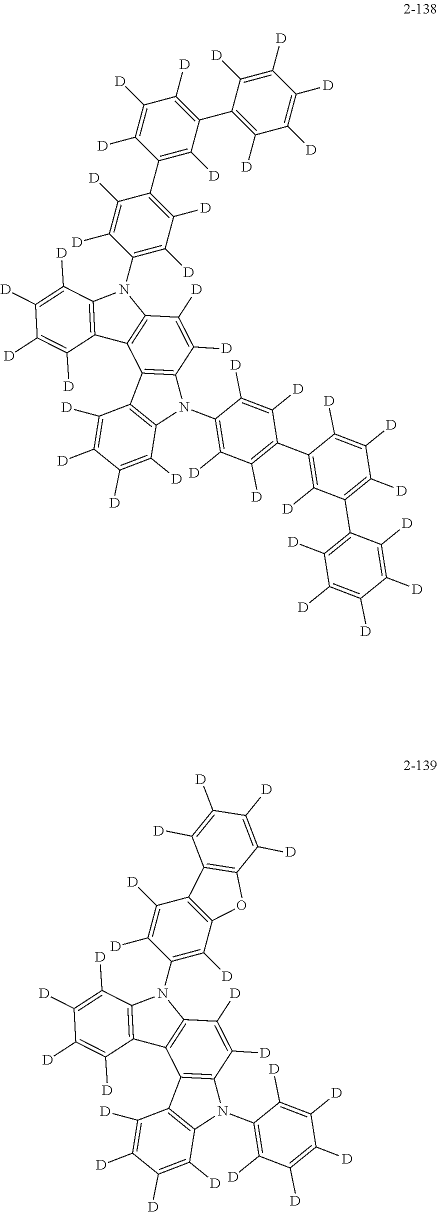 [SMILES: [2H]c1c([2H])c([2H])c(-c2c([2H])c([2H])c([2H])c(-c3c([2H])c([2H])c(-n4c5c([2H])c([2H])c([2H])c([2H])c5c5c6c7c([2H])c([2H])c([2H])c([2H])c7n(-c7c([2H])c([2H])c(-c8c([2H])c([2H])c([2H])c(-c9c([2H])c([2H])c([2H])c([2H])c9[2H])c8[2H])c([2H])c7[2H])c6c([2H])c([2H])c54)c([2H])c3[2H])c2[2H])c([2H])c1[2H] ; [2H]c1c([2H])c([2H])c(-n2c3c([2H])c([2H])c([2H])c([2H])c3c3c4c5c([2H])c([2H])c([2H])c([2H])c5n(-c5c([2H])c([2H])c6c(oc7c([2H])c([2H])c([2H])c([2H])c76)c5[2H])c4c([2H])c([2H])c32)c([2H])c1[2H]]

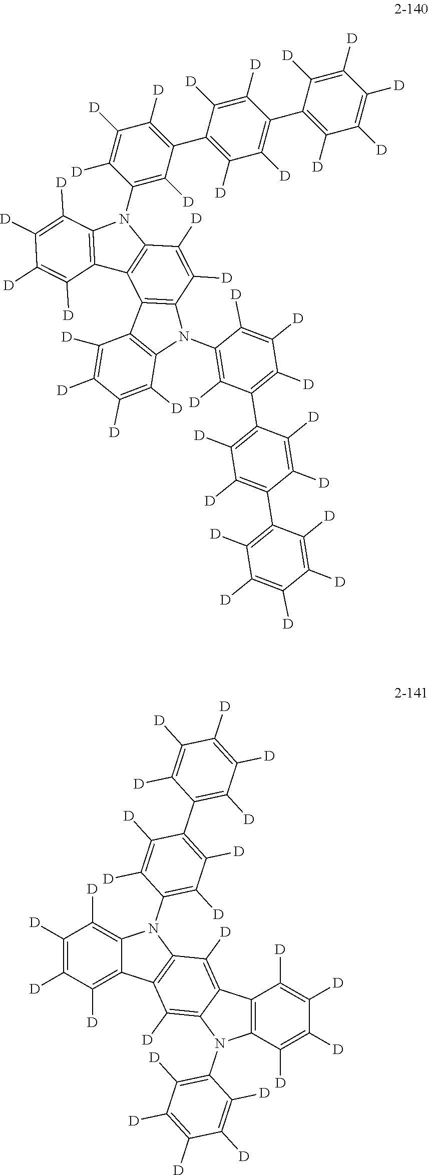 [SMILES: [2H]c1c([2H])c([2H])c(-c2c([2H])c([2H])c(-c3c([2H])c([2H])c([2H])c(-n4c5c([2H])c([2H])c([2H])c([2H])c5c5c6c7c([2H])c([2H])c([2H])c([2H])c7n(-c7c([2H])c([2H])c([2H])c(-c8c([2H])c([2H])c(-c9c([2H])c([2H])c([2H])c([2H])c9[2H])c([2H])c8[2H])c7[2H])c6c([2H])c([2H])c54)c3[2H])c([2H])c2[2H])c([2H])c1[2H] ; [2H]c1c([2H])c([2H])c(-c2c([2H])c([2H])c(-n3c4c([2H])c([2H])c([2H])c([2H])c4c4c([2H])c5c(c([2H])c43)c3c([2H])c([2H])c([2H])c([2H])c3n5-c3c([2H])c([2H])c([2H])c([2H])c3[2H])c([2H])c2[2H])c([2H])c1[2H]]

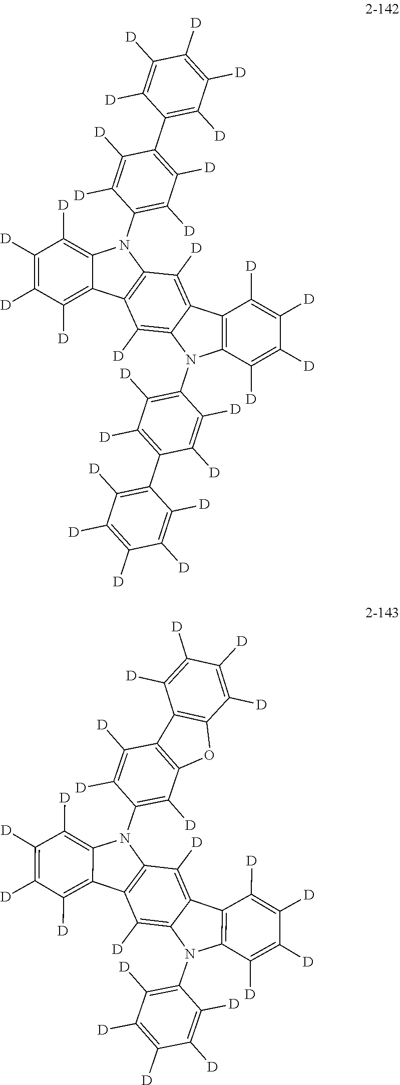 [SMILES: [2H]c1c([2H])c([2H])c(-c2c([2H])c([2H])c(-n3c4c([2H])c([2H])c([2H])c([2H])c4c4c([2H])c5c(c([2H])c43)c3c([2H])c([2H])c([2H])c([2H])c3n5-c3c([2H])c([2H])c(-c4c([2H])c([2H])c([2H])c([2H])c4[2H])c([2H])c3[2H])c([2H])c2[2H])c([2H])c1[2H] ; [2H]c1c([2H])c([2H])c(-n2c3c([2H])c([2H])c([2H])c([2H])c3c3c([2H])c4c(c([2H])c32)c2c([2H])c([2H])c([2H])c([2H])c2n4-c2c([2H])c([2H])c3c(oc4c([2H])c([2H])c([2H])c([2H])c43)c2[2H])c([2H])c1[2H]]

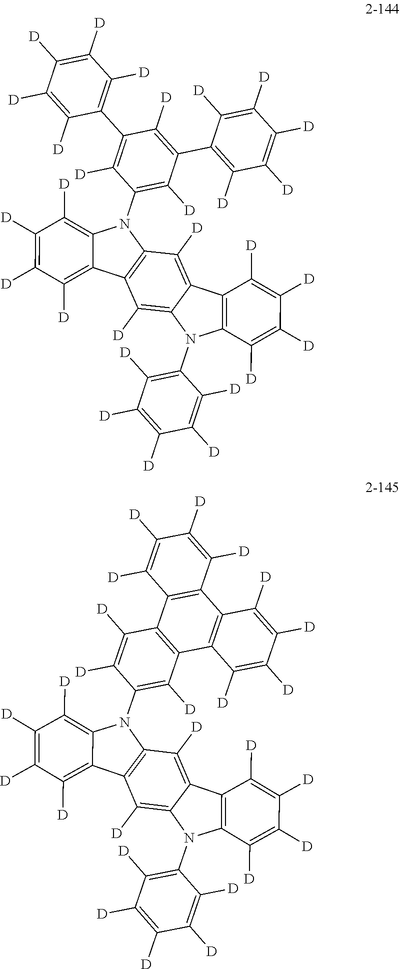 [SMILES: [2H]c1c([2H])c([2H])c(-c2c([2H])c(-c3c([2H])c([2H])c([2H])c([2H])c3[2H])c([2H])c(-n3c4c([2H])c([2H])c([2H])c([2H])c4c4c([2H])c5c(c([2H])c43)c3c([2H])c([2H])c([2H])c([2H])c3n5-c3c([2H])c([2H])c([2H])c([2H])c3[2H])c2[2H])c([2H])c1[2H] ; [2H]c1c([2H])c([2H])c(-n2c3c([2H])c([2H])c([2H])c([2H])c3c3c([2H])c4c(c([2H])c32)c2c([2H])c([2H])c([2H])c([2H])c2n4-c2c([2H])c([2H])c3c4c([2H])c([2H])c([2H])c([2H])c4c4c([2H])c([2H])c([2H])c([2H])c4c3c2[2H])c([2H])c1[2H]]

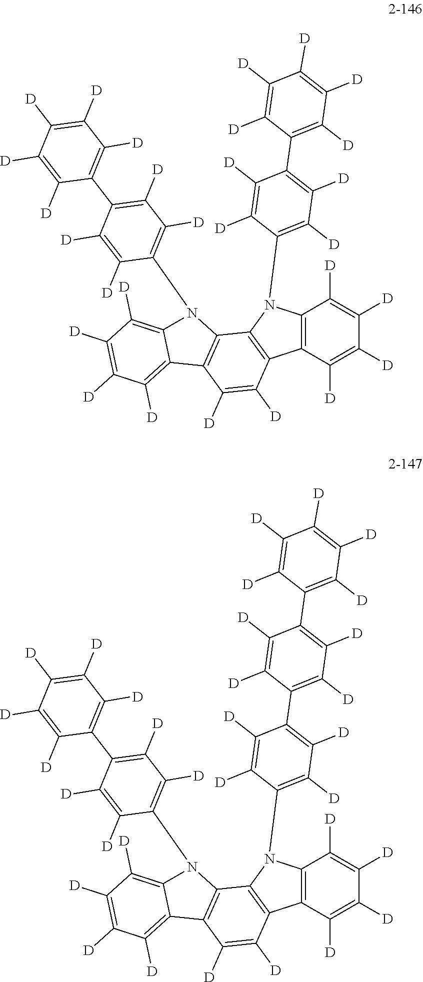 [SMILES: [2H]c1c([2H])c([2H])c(-c2c([2H])c([2H])c(-n3c4c([2H])c([2H])c([2H])c([2H])c4c4c([2H])c([2H])c5c6c([2H])c([2H])c([2H])c([2H])c6n(-c6c([2H])c([2H])c(-c7c([2H])c([2H])c([2H])c([2H])c7[2H])c([2H])c6[2H])c5c43)c([2H])c2[2H])c([2H])c1[2H] ; [2H]c1c([2H])c([2H])c(-c2c([2H])c([2H])c(-c3c([2H])c([2H])c(-n4c5c([2H])c([2H])c([2H])c([2H])c5c5c([2H])c([2H])c6c7c([2H])c([2H])c([2H])c([2H])c7n(-c7c([2H])c([2H])c(-c8c([2H])c([2H])c([2H])c([2H])c8[2H])c([2H])c7[2H])c6c54)c([2H])c3[2H])c([2H])c2[2H])c([2H])c1[2H]]

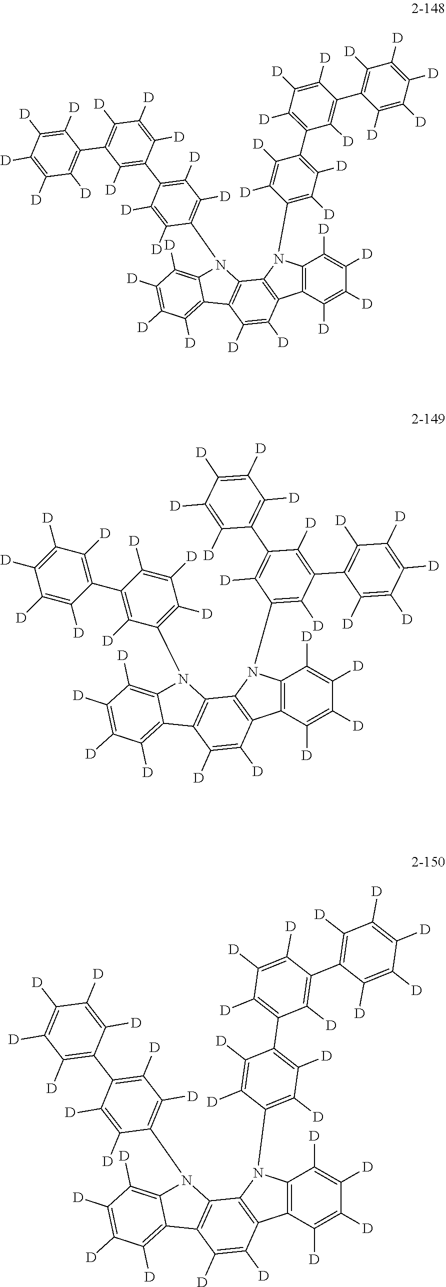

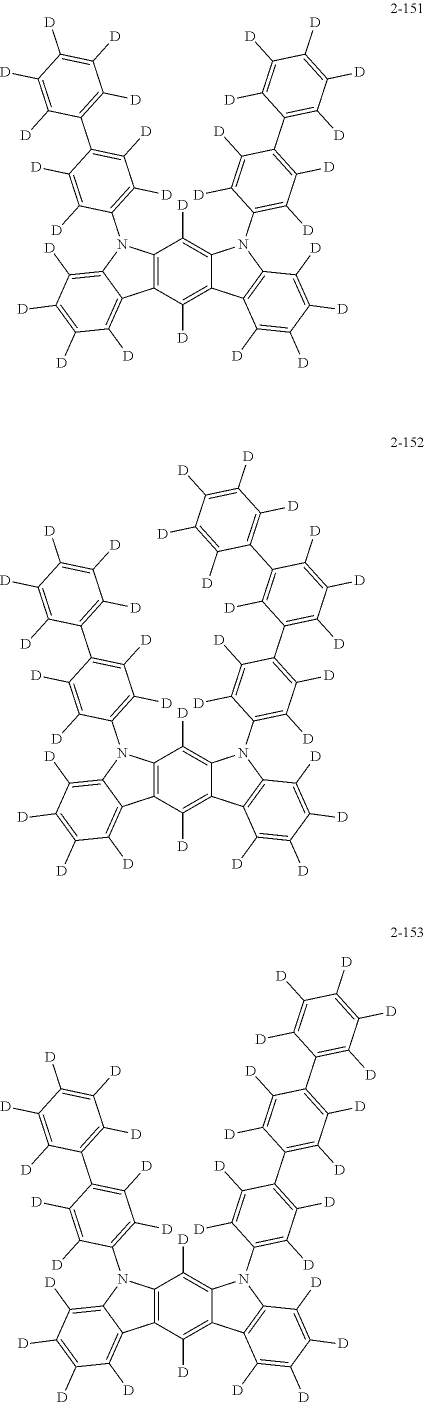

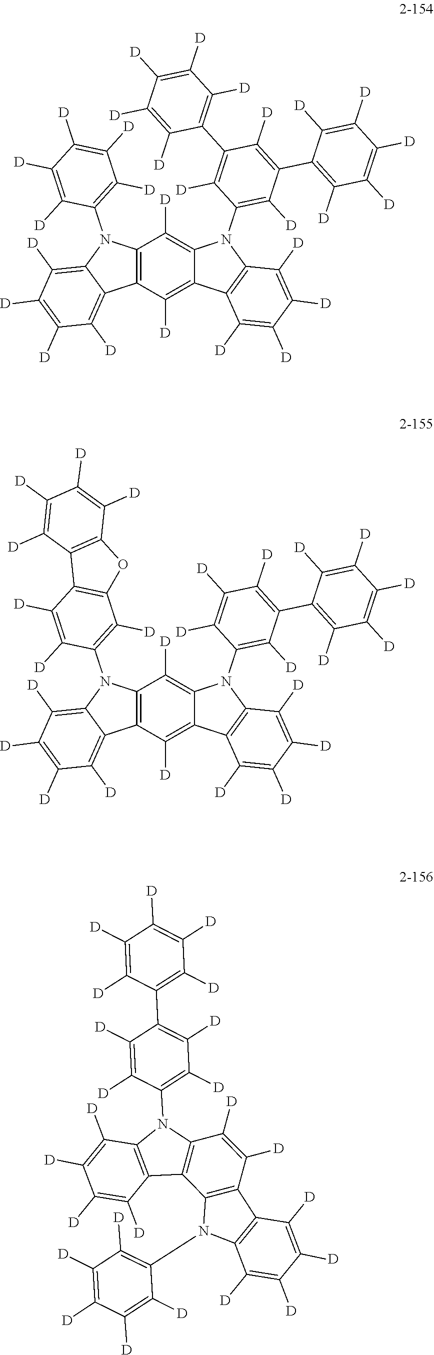

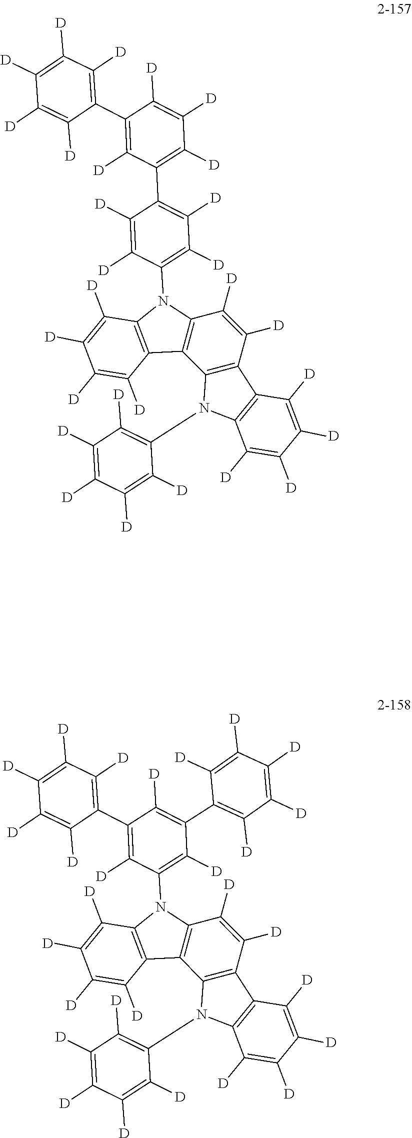 [SMILES: [2H]c1c([2H])c([2H])c(-c2c([2H])c([2H])c([2H])c(-c3c([2H])c([2H])c(-n4c5c([2H])c([2H])c([2H])c([2H])c5c5c4c([2H])c([2H])c4c6c([2H])c([2H])c([2H])c([2H])c6n(-c6c([2H])c([2H])c([2H])c([2H])c6[2H])c45)c([2H])c3[2H])c2[2H])c([2H])c1[2H] ; [2H]c1c([2H])c([2H])c(-c2c([2H])c(-c3c([2H])c([2H])c([2H])c([2H])c3[2H])c([2H])c(-n3c4c([2H])c([2H])c([2H])c([2H])c4c4c3c([2H])c([2H])c3c5c([2H])c([2H])c([2H])c([2H])c5n(-c5c([2H])c([2H])c([2H])c([2H])c5[2H])c34)c2[2H])c([2H])c1[2H]]

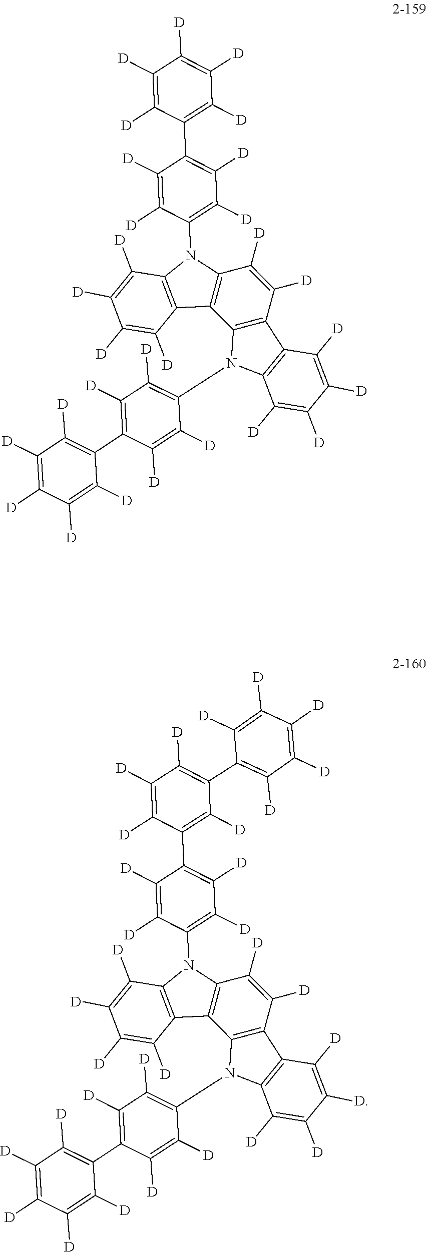

13. A composition for forming an organic material layer of an organic light emitting device, comprising the heterocyclic compound of claim 1.

14. The composition of claim 13, wherein the composition comprises a compound of the following Chemical Formula 2 or 3: 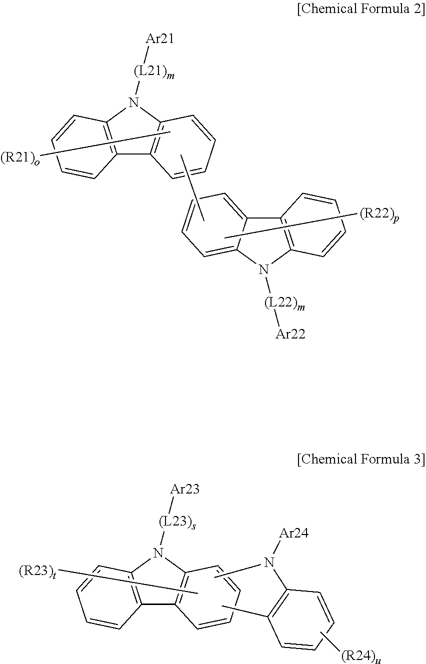
in Chemical Formulae 2 and 3,
L21 to L23 are each independently a direct bond; a substituted or unsubstituted C6 to C60 arylene group; or a substituted or unsubstituted C2 to C60 heteroarylene group,
m, n, and s are each an integer from 1 to 3, and when m, n, and s are each 2 or greater, substituents in the parenthesis are the same as or different from each other,
Ar21 to Ar24 are each independently a substituted or unsubstituted C6 to C60 aryl group; or a substituted or unsubstituted C2 to C60 heteroaryl group,
R21 to R24 are each independently hydrogen; deuterium; a halogen group; a cyano group; a substituted or unsubstituted C1 to C60 alkyl group; a substituted or unsubstituted C6 to C60 aryl group; or a substituted or unsubstituted C2 to C60 heteroaryl group, and
o and p are each an integer from 1 to 7, tis an integer from 1 to 6, u is an integer from 1 to 4, and when o, p, t, and u are each 2 or greater, substituents in the parenthesis are the same as or different from each other.

15. The composition of claim 14, wherein a weight ratio of the heterocyclic compound and the compound of Chemical Formula 2 or 3 is 1:10 to 10:1.
